(19) United States
(12) Patent Application Publication
(10) Pub. No.: US 2025/0197420 A1
(43) Pub. Date: Jun. 19, 2025

(54) MACROCYCLIC COMPOUNDS, COMPOSITIONS, AND METHODS OF USING THEREOF

(71) Applicants: Sionna Therapeutics Inc., Waltham, MA (US); Genzyme Corporation, Cambridge, MA (US)

(72) Inventors: Junkai Liao, Andover, MA (US); John E. Macor, Washington Crossing, PA (US); George Topalov, Pittsburgh, PA (US); Mark Munson, Acton, MA (US); Sukanthini Thurairatnam, Dover, MA (US); Bradford Hirth, Littleton, MA (US); Zhongli Gao, Waltham, MA (US); Gregory Donald Hurlbut, Boxborough, MA (US); Andrew Good, Bideford (GB); Roy Vaz, Southborough, MA (US); Jinyu Liu, Union City, CA (US); Yi Li, Weston, MA (US); Anatoly Ruvinsky, Lexington, MA (US); Michael Kothe, Medway, MA (US); David Borcherding, Bangor, PA (US); Lothar Schwink, Frankfurt am Main (DE); Heiner Glombik, Frankfurt am Main (DE); Stefan Guessregen, Frankfurt am Main (DE); Michael Podeschwa, Frankfurt am Main (DE); Nils Rackelmann, Frankfurt am Main (DE); Sven Ruf, Frankfurt am Main (DE)

(21) Appl. No.: 19/071,528

(22) Filed: Mar. 5, 2025

Related U.S. Application Data
(63) Continuation of application No. PCT/US2023/073551, filed on Sep. 6, 2023.
(60) Provisional application No. 63/404,443, filed on Sep. 7, 2022.

(51) Int. Cl. C07D 498/18; A61P 11/00; C07D 515/18

(52) U.S. Cl. CPC C07D 498/18; A61P 11/00; C07D 515/18

(57) ABSTRACT

The present disclosure includes, among other things, CFTR modulators, pharmaceutical compositions, and methods of making and using the same.

This application is a continuation of International Application No. PCT/US2023/073551, filed Sep. 6, 2023, which claims the benefit of and priority to U.S. Provisional Patent Application No. 63/404,443, filed Sep. 7, 2022, each of which are hereby incorporated by reference in their entireties for all purposes.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate, and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 70% of cystic fibrosis patients have a deletion at residue 508 in at least one CFTR allele. The loss of this key phenylalanine renders NBD1 conformationally unstable at physiological temperature and compromises the integrity of the interdomain interface between NDB1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

The present disclosure includes a compound of formula A:

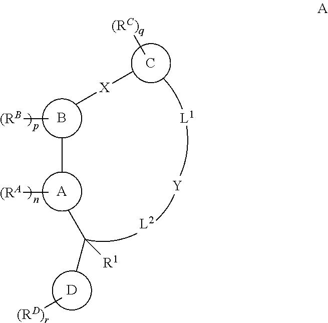

or a pharmaceutically acceptable salt thereof. Additionally, the present disclosure includes, among other things, pharmaceutical compositions, methods of using and methods of making a compound of formula A.

### DETAILED DESCRIPTION

In some embodiments, the present disclosure includes a compound of Formula A:

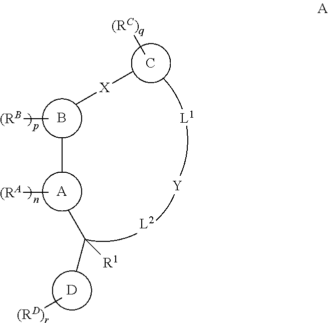

or a pharmaceutically acceptable salt thereof,
   wherein
L¹ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl,

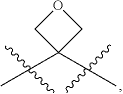 [SMILES: *C1(*)COC1]

or optionally substituted C₂ alkenylene;
L² is an optionally substituted C₁₋₆ alkylene chain wherein 1-3 of the methylene units is optionally and independently replaced by —C(CD₃)₂-, —O—, —N(R²), —C(O)—, —S—, —S(O)—, an optionally substituted 3-6 membered carbocyclyl, or optionally substituted C₂ alkenylene; Ring A is optionally substituted 5-membered heteroaryl comprising 1-3 heteroatoms selected from the group consisting of N, O or S;
Ring B is optionally substituted phenyl or optionally substituted 6-membered heteroaryl;
Ring C is optionally substituted phenyl or optionally substituted 5-10-membered heteroaryl;
Ring D is optionally substituted phenyl or optionally substituted 5-6-membered heteroaryl;
X is selected from the group consisting of —O—, —S—, —CH₂—, —C(OH)H—, —SO—, —CO—, —SO₂—, —CFH—, —CF₂—, and —N(R²)—;
each R^{A} is independently selected from the group consisting of halogen, cyano, optionally substituted C₁-C₆ aliphatic, optionally substituted C₁-C₆ alkoxy, and —CD₃;
each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy;
each R^{C} is independently selected from the group consisting of halogen, cyano, optionally substituted C₁-C₆ aliphatic or optionally substituted C₁-C₆ alkoxy;
each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
each R^{d} is independently selected from the group consisting of hydrogen, —OH, —CD₃, —C(O)N(R²)₂, C(O)OR², —OR², —N(R²)₂, optionally substituted C₁-C₆ aliphatic, optionally substituted 5-6-membered heteroaryl, and optionally substituted 3-6-membered heterocyclyl comprising 1-3 heteroatoms selected from the group consisting of N, O or S
R¹ is selected from the group consisting of hydrogen, cyano, —OR², —(CH₂)₀₋₃N(R²)₂, optionally substituted C₁-C₃ aliphatic, and —CD₃;
each R² is independently selected from hydrogen, optionally substituted C₁-C₆ aliphatic, —OH, C₁-C₆ alkoxy, —S(O)₂(optionally substituted C₁-C₆ aliphatic);
n is 0, 1, 2 or 3;
p is 0, 1, 2, 3 or 4;
q is 0, 1 or 2; and
r is 0, 1, 2, 3, 4 or 5.

In some embodiments, the present disclosure includes a compound of Formula I:

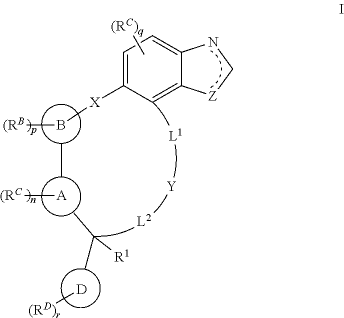

or a pharmaceutically acceptable salt thereof,
   wherein
L¹ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl,

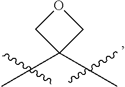 [SMILES: *C1(*)COC1]

or optionally substituted C₂ alkenylene;
L² is an optionally substituted C₁₋₆ alkylene chain wherein 1-3 of the methylene units is optionally and independently replaced by —C(CD₃)₂-, —O—, —N(R²), —C(O)—, —S—, —S(O)—, an optionally substituted 3-6 membered carbocyclyl, or optionally substituted C₂ alkenylene;
Ring A is an optionally substituted 5-membered heteroaryl comprising 1-4 heteroatoms selected from the group consisting of N, O or S;
Ring B is optionally substituted phenyl or optionally substituted 6-membered heteroaryl;
Ring D is optionally substituted phenyl or optionally substituted 5-6-membered heteroaryl;
X is selected from the group consisting of —O—, —S—, —CH₂—, —C(OH)H—, —SO—, —CO—, —SO₂—, —CFH—, —CF₂—, and —N(R²)—;
Y is selected from the group consisting of —S(O)₂N(R²)—, —OC(O)N(R²)—, and —C(O)N(R²)—;
each R^{A} is independently selected from the group consisting of halogen, cyano, optionally substituted C₁-C₆ aliphatic, optionally substituted C₁-C₆ alkoxy, and —CD₃;
each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy;
each R^{C} is independently selected from the group consisting of hydrogen, halogen, cyano, optionally substituted C₁-C₆ aliphatic or optionally substituted C₁-C₆ alkoxy;
each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
   wherein two instances of R^{D} may be taken together to form an optionally substituted 5-7 membered carbocyclic ring, optionally substituted 5-6-membered heteroaryl, and optionally substituted 3-6-membered heterocyclyl comprising 1-3 heteroatoms selected from the group consisting of N, O or S;
each R^{d} is independently selected from the group consisting of hydrogen, —OH, —CD₃, —C(O)N(R²)₂, C(O)OR², —OR², —N(R²)₂, —S(O)₂R² optionally substituted C₁-C₆ aliphatic, optionally substituted 5-6-membered heteroaryl, and optionally substituted 3-6-membered heterocyclyl comprising 1-3 heteroatoms selected from the group consisting of N, O or S;
R¹ is selected from the group consisting of hydrogen, cyano, —OR², —(CH₂)₀₋₃N(R²)₂, optionally substituted C₁-C₃ aliphatic, 3-6-membered heterocyclyl comprising 1-3 heteroatoms selected from the group consisting of N, O or S, and —CD₃;
each R² is independently selected from hydrogen, optionally substituted C₁-C₆ aliphatic, —OH, C₁-C₆ alkoxy, —S(O)₂(optionally substituted C₁-C₆ aliphatic);
Z is —CH═, —N═ or —NH—;
n is 0, 1, 2 or 3;
p is 0, 1, 2, 3 or 4;
q is 1 or 2; and
r is 0, 1, 2, 3, 4 or 5.

In some embodiments, the present disclosure includes a compound of formula I-a, I-b, I-c, or I-d:

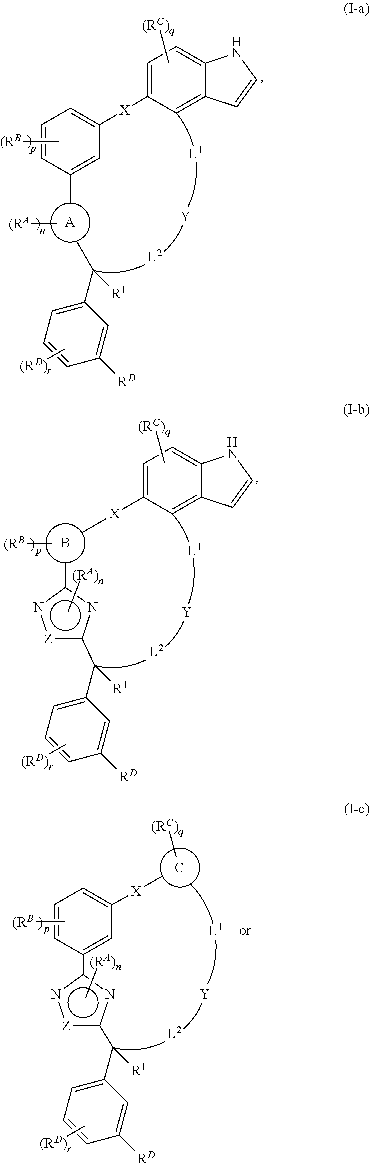

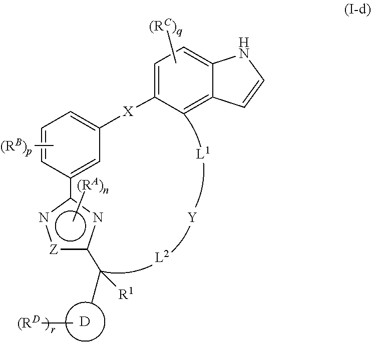

or a pharmaceutically acceptable salt thereof,
   wherein Ring A, Ring B, Ring C, Ring D, L¹, L², X, Y, Z, R¹, R^{A}, R^{B}, R^{C}, R^{D}, n, p, q, and r are defined herein.

In some embodiments, the present disclosure includes a compound of formula (I-a1), (I-a2), (I-a3), (I-a4), or (I-a5):

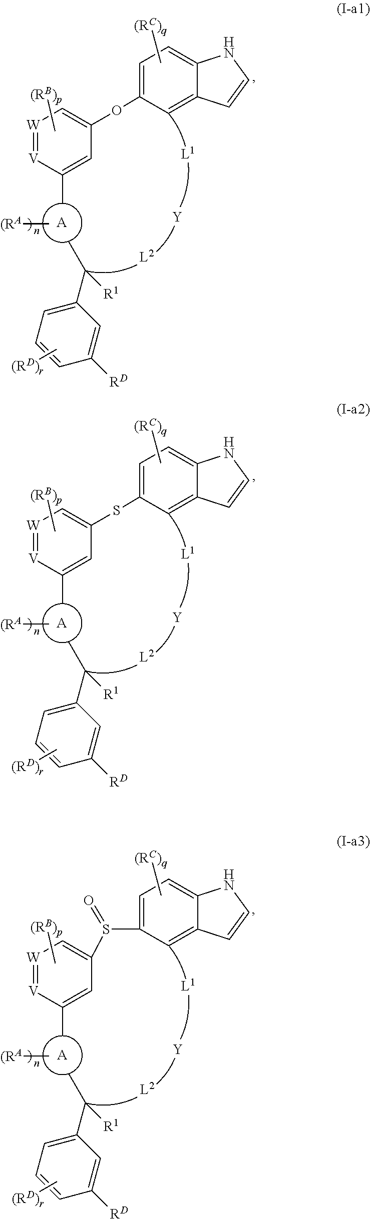

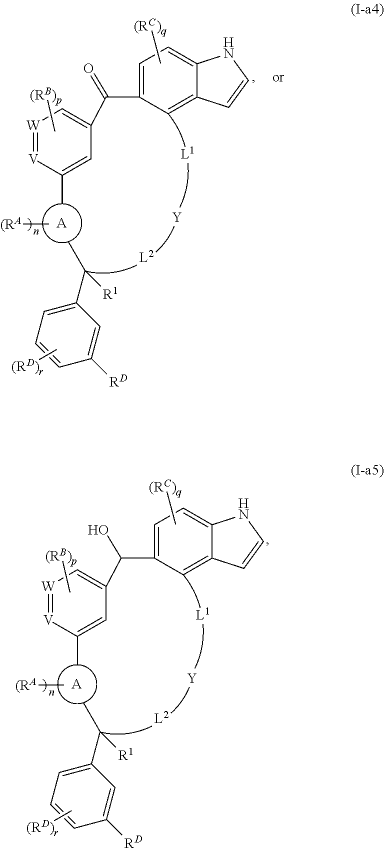

or a pharmaceutically acceptable salt thereof,
wherein Ring A, L¹, L², W, V, Y, R¹, R^{A}, R^{B}, R^{C}, R^{D}, n, p, q, and r are defined herein.

In some embodiments, the present disclosure includes a compound of formula (1-d1), (I-d2), (I-d3), (I-d4), or (I-d5)

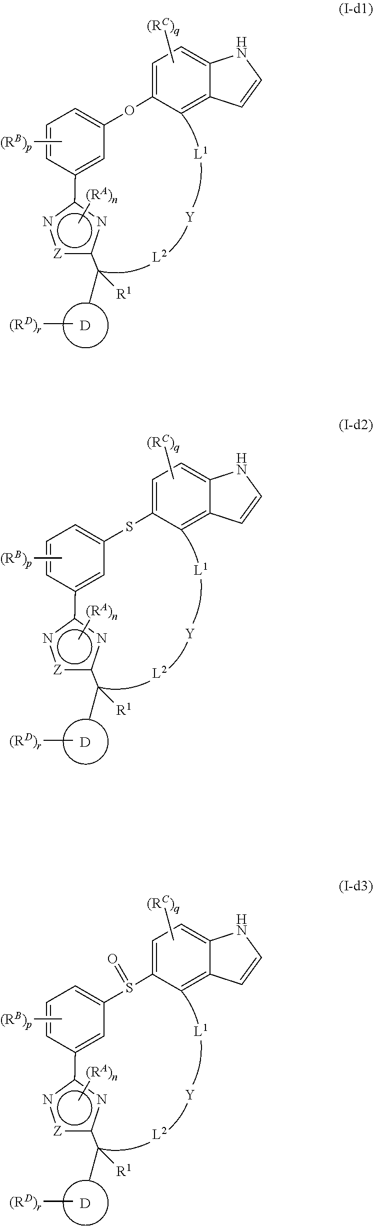

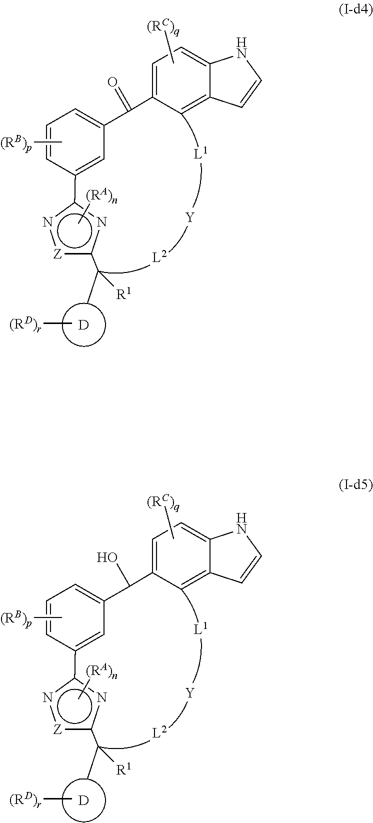

or a pharmaceutically acceptable salt thereof,
   wherein Ring D, L¹, L², Y, Z, R¹, RA, RB, R^{C}, RD, n, p, q, and r are defined herein.

In some embodiments, the present disclosure includes a compound of formula (I-e):

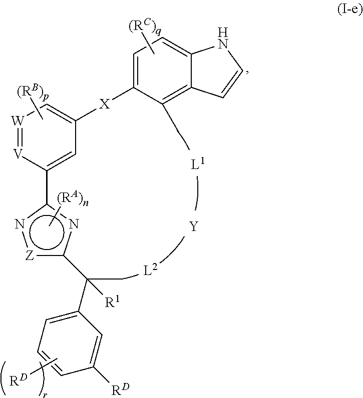

or a pharmaceutically acceptable salt thereof,
   wherein L¹, L², V, W, X, Y, Z, R¹, R^{A}, R^{B}, R^{C}, R^{D}, m, n, p, q, and r are defined herein.

In some embodiments, the present disclosure includes a compound of formula I-f:

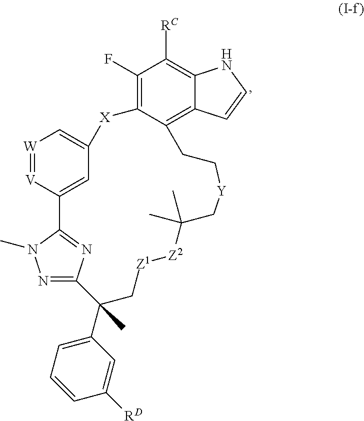

or a pharmaceutically acceptable salt thereof,
   wherein V, W, X, Y, Z¹, Z², R^{C}, and R^{D} are defined herein.

In some embodiments, the present disclosure includes a compound of formula I-g:

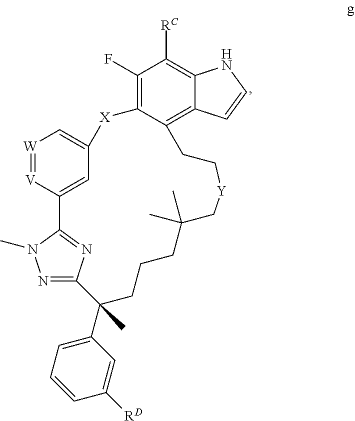

or a pharmaceutically acceptable salt thereof,
   wherein V, W, X, Y, R^{C}, and R^{D} are defined herein.

In some embodiments, the present disclosure includes a compound of formula (I-g1) or (I-g2)

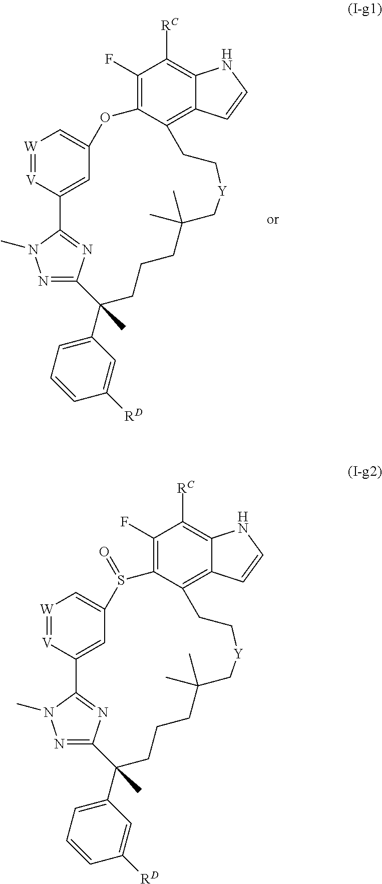

or a pharmaceutically acceptable salt thereof, wherein V, W, Y, R^{C}, and R^{D} are defined herein.

In some embodiments, the present disclosure includes a compound of formula I-h:

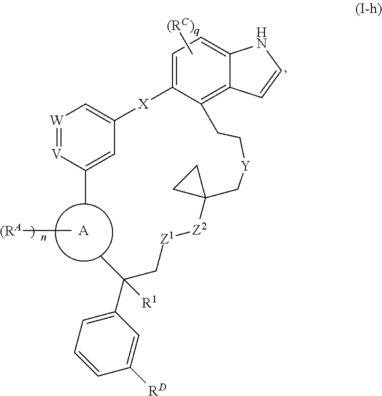

or a pharmaceutically acceptable salt thereof,
   wherein Ring A, L, L², W, V, X, Y, Z¹, Z², R, R^{A}, R^{C}, R^{D}, n, p, q, and r are defined herein.

In some embodiments, the present disclosure includes a compound of formula I-i:

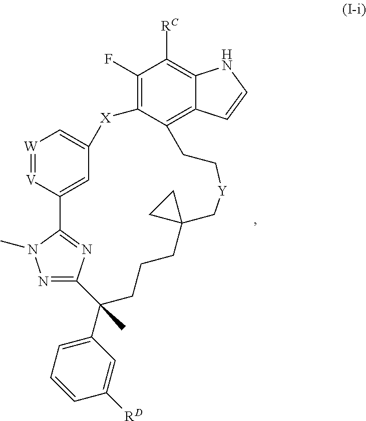

or a pharmaceutically acceptable salt thereof,
   wherein V, W, X, Y, R¹, R^{C}, and R^{D} are defined herein.

### Ring A

In some embodiments, Ring A is an optionally substituted 5-membered heteroaryl comprising 1-4 heteroatoms selected from the group consisting of N, O or S. In some embodiments, Ring A is an optionally substituted 5-membered heteroaryl comprising 1-3 heteroatoms selected from the group consisting of N and O. In some embodiments, Ring A is an optionally substituted 5-membered heteroaryl comprising 1 nitrogen atom. In some embodiments, Ring A is an optionally substituted 5-membered heteroaryl comprising 2 nitrogen atoms. In some embodiments, Ring A is an optionally substituted 5-membered heteroaryl comprising 3 nitrogen atoms.

In some embodiments, Ring A is selected from the group consisting of furan, pyrrole, thiophene, pyrazole, oxazole, thiazole, imidazole, triazole, tetrazole, oxadiazole, and thiadiazole. In some embodiments, Ring A is selected from the group consisting of furan, pyrrole, thiophene, pyrazole, oxazole, thiazole, imidazole, triazole, tetrazole, oxadiazole, and thiadiazole. In some embodiments, Ring A is selected from the ground consisting of imidazole, pyrazole, and triazole. In some embodiments, Ring A is selected from the group consisting of imidazole and triazole.

In some embodiments, Ring A is

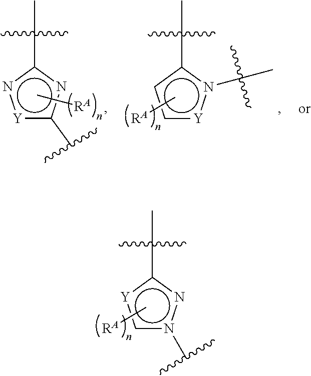

wherein Y is C or N.

In some embodiments, Ring A is selected from the ground consisting of

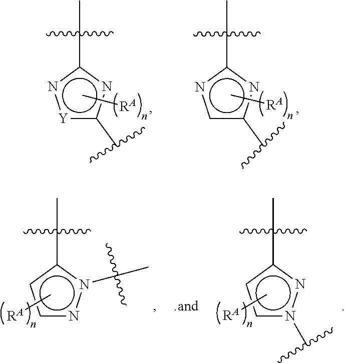

In some embodiments, Ring A is selected from the group consisting of

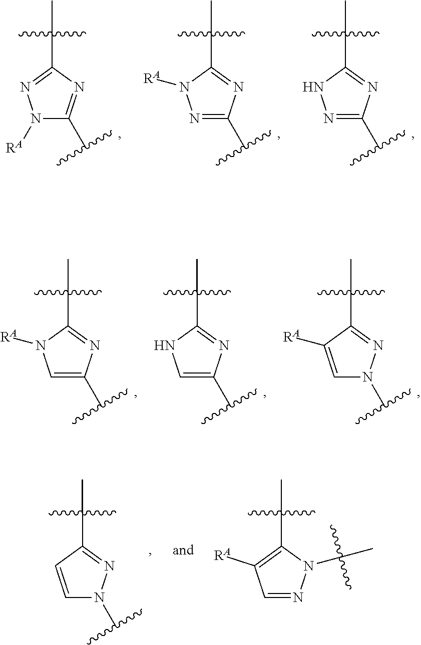

### Ring B

In some embodiments, Ring B is optionally substituted phenyl or optionally substituted 6-membered heteroaryl. In some embodiments, Ring B is optionally substituted phenyl, optionally substituted pyridine, or optionally substituted pyridone. In some embodiments, Ring B is optionally substituted phenyl. In some embodiments, Ring B is optionally substituted pyridyl. In some embodiments, Ring B is optionally substituted pyridone.

In some embodiments, Ring B is

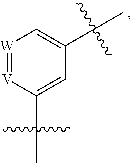

wherein
W is —CH═, —C(R^{B})═ or —N═; and
V is —CH═, —C(R^{B})═ or —N═.

In some embodiments, Ring B is selected from the group consisting of

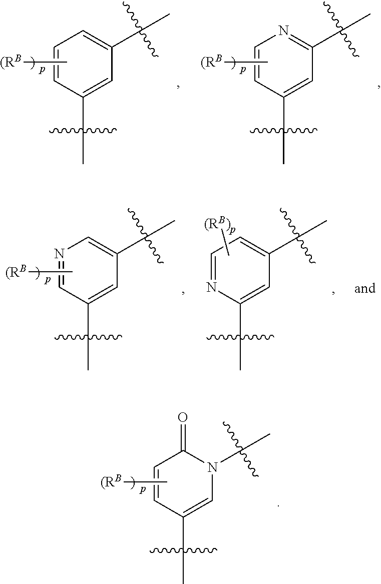

In some embodiments, Ring B is selected from the group consisting of

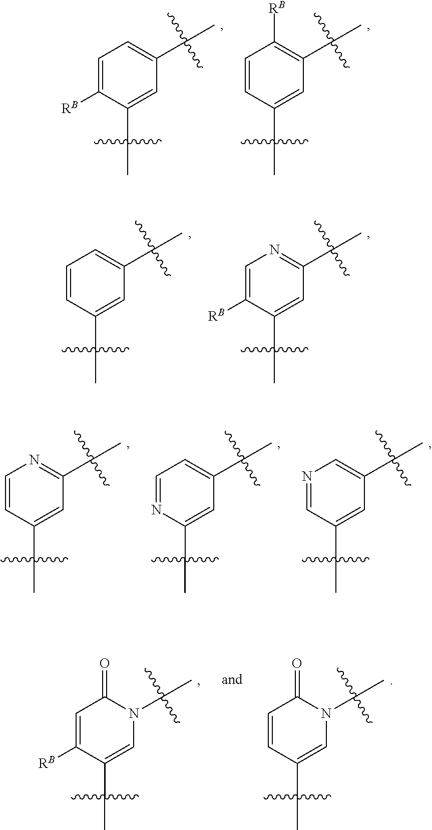

In some embodiments, Ring B is

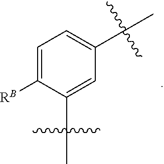

In some embodiments, Ring B is

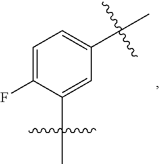 [SMILES: *c1ccc(F)c(*)c1]

### Ring C

In some embodiments, Ring C is optionally substituted 5-10-membered heteroaryl comprising 1-3 heteroatoms selected from the group consisting of O, S, and N. In some embodiments, Ring C is optionally substituted 9-10-membered heteroaryl comprising 1-3 heteroatoms selected from the group consisting of O, S, and N. In some embodiments, Ring C is selected from the group consisting of optionally substituted indole, optionally substituted indazole, optionally substituted benzimidazole, optionally substituted 6-azaindole, and optionally substituted 7-azaindole. In some embodiments, Ring C is optionally substituted indole.

In some embodiments, Ring C is

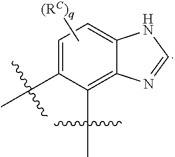

In some embodiments, Ring C is

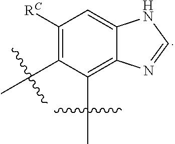

In some embodiments, Ring C is

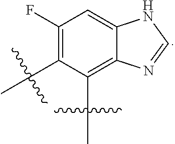 [SMILES: *c1c(F)cc2[nH]cnc2c1*]

In some embodiments, Ring C is

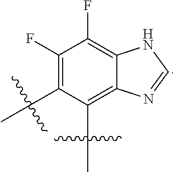 [SMILES: *c1c(F)c(F)c2[nH]cnc2c1*]

In some embodiments, Ring C is

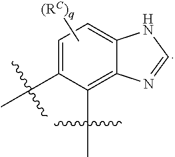

In some embodiments, Ring C is

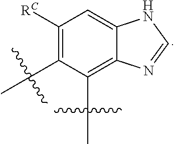

In some embodiments, Ring C is

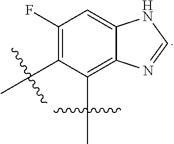 [SMILES: *c1c(F)cc2[nH]cnc2c1*]

### Ring D

In some embodiments, Ring D is optionally substituted phenyl or optionally substituted 5-6-membered heteroaryl comprising 1-3 heteroatoms selected from the group consisting of O, S, and N. In some embodiments, Ring D is optionally substituted phenyl. In some embodiments, Ring D is optionally substituted 5-6-membered heteroaryl comprising 1-3 heteroatoms selected from the group consisting of O, S, and N. In some embodiments, Ring D is optionally substituted 5-membered heteroaryl comprising 1-3 heteroatoms selected from the group consisting of O, S, and N. In some embodiments, Ring D is 6-membered heteroaryl comprising 1-3 heteroatoms selected from the group consisting of O, S, and N. In some embodiments Ring D is optionally substituted pyridine.

In some embodiments, Ring D is

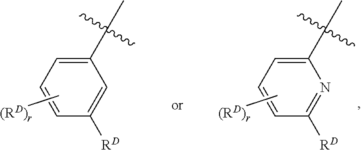

In some embodiments, Ring D is

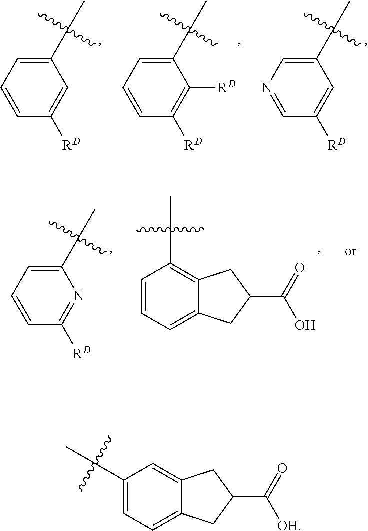

In some embodiments, Ring D is

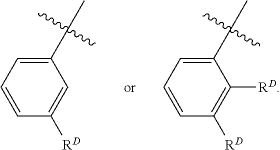

In some embodiments, Ring D is

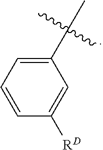

In some embodiments, Ring D is

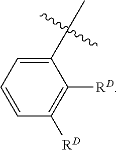

In some embodiments, Ring D is

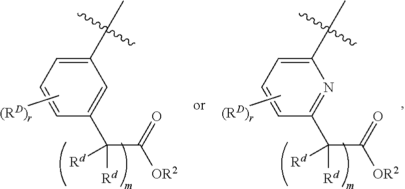

In some embodiments, Ring D is

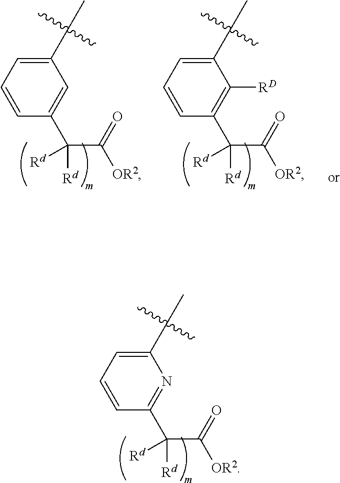

In some embodiments, Ring D is

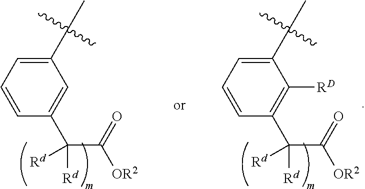

In some embodiments, Ring D is

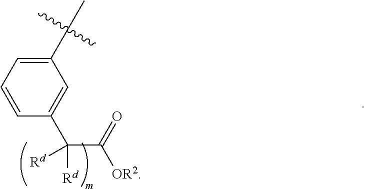

In some embodiments, Ring D is

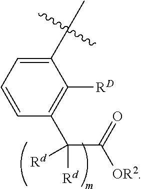

L¹ and L²

In some embodiments, L¹ is an optionally substituted C₁₋₆ alkylene chain wherein 1-3 of the methylene units is optionally and independently replaced by —O—, —N(R²), —C(O)—, —S—, —S(O)—, an optionally substituted 3-6 membered carbocyclyl,

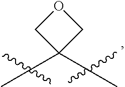 [SMILES: *C1(*)COC1]

or optionally substituted C₂ alkenylene. In some embodiments, L² is an optionally substituted C₁₋₆ alkylene chain wherein 1-3 of the methylene units is optionally and independently replaced by —C(CD₃)₂-, —O—, —N(R²), —C(O)—, —S—, —S(O)—, an optionally substituted 3-6 membered carbocyclyl, or optionally substituted C₂ alkenylene. In some embodiments, L¹ is an optionally substituted C₁₋₆ alkylene chain wherein 1-3 of the methylene units is optionally and independently replaced by —O—, —N(R²), —C(O)—, or

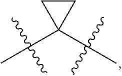 [SMILES: *C1(*)CC1]

and L² is an optionally substituted C₁₋₆ alkylene chain wherein 1-3 of the methylene units is optionally and independently replaced by —C(CD₃)₂-, —O—, —N(R²), —C(O)—, or

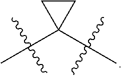 [SMILES: *C1(*)CC1]

In some embodiments, L¹ is an optionally substituted C₁₋₆ alkylene chain and L² is an optionally substituted C₁₋₆ alkylene chain, wherein one of the methylene units of L² is optionally replaced with —O—. In some embodiments, L¹ is a C₁₋₆ alkylene chain substituted with 1-3 instances of methyl, and L² is C₁₋₆ alkylene chain, wherein one of the methylene units of L² is optionally replaced with —O— and wherein L² is optionally substituted with 1-3 instances of methyl. In some embodiments, L¹ is an unsubstituted C₂ alkylene chain. In some embodiments, L² is a C₅ alkylene chain, wherein one of the methylene units of L² is optionally replaced with —O— and wherein L² is optionally substituted with 1-3 instances of methyl. In some embodiments, L² is a C₅ alkylene chain, wherein L² is optionally substituted with 1-3 instances of methyl. In some embodiments, L² is optionally substituted with 1-3 instances of methyl.

In some embodiments, L¹ is

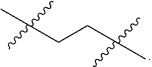 [SMILES: *CC*]

In some embodiments, L² is

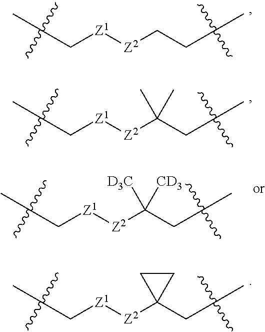

wherein Z¹ is —CH₂—, —CF₂—, —C(O)—, or —O—; and
Z² is —CH₂—, —CF₂—, —C(O)—, or —O—.

In some embodiments, L² is

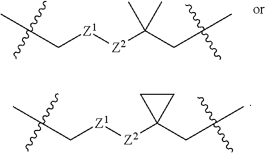

wherein Z¹ is —CH₂— or —O—; and
Z² is —CH₂— or —O—.

In some embodiments, L² is

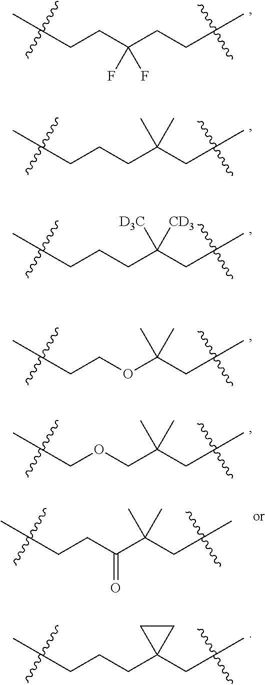

In some embodiments, L² is

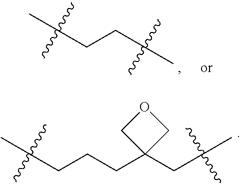

In some embodiments, Z¹ is —CH₂—, and Z² is —O—. In some embodiments, Z¹ is —O—, and Z¹ is —CH₂—.

### R^{A}

In some embodiments, each R^{A} is independently selected from the group consisting of halogen, cyano, optionally substituted C₁-C₆ aliphatic, optionally substituted C₁-C₆ alkoxy, and —CD₃. In some embodiments, each R^{A} is independently selected from cyano and optionally substituted C₁-C₆ aliphatic. In some embodiments, each R^{A} is independently selected from cyano and optionally substituted C₁-C₃ aliphatic. In some embodiments, each R^{A} is independently optionally substituted C₁-C₃ aliphatic. In some embodiments, R^{A} is methyl.

### R^{B}

In some embodiments, each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy. In some embodiments, each R^{B} is independently selected from halogen and cyano. In some embodiments, each R^{B} is independently selected from the group consisting of halogen and optionally substituted C₁-C₃ alkyl. In some embodiments, each R^{B} is independently selected from halogen. In some embodiments, R^{B} is fluoro.

### R^{C}

In some embodiments, each R^{C} is independently selected from the group consisting of halogen, cyano, optionally substituted C₁-C₆ aliphatic or optionally substituted C₁-C₆ alkoxy. In some embodiments, each R^{C} is independently selected from halogen, cyano, and optionally substituted C₁-C₆ alkyl. In some embodiments, each R^{C} is independently selected from halogen. In some embodiments, R^{C} is fluoro.

### R^{D}

In some embodiments, each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d}.
wherein two instances of R^{D} may be taken together to form an optionally substituted 5-7 membered carbocyclic ring, optionally substituted 5-6-membered heteroaryl, and optionally substituted 3-6-membered heterocyclyl comprising 1-3 heteroatoms selected from the group consisting of N, O or S;

In some embodiments, each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d}.

In some embodiments, each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S.

In some embodiments, each R^{D} is independently selected from the group consisting of halogen, OR², and optionally substituted C₁-C₆ aliphatic. In some embodiments, each R^{D} is independently selected from the group consisting of halogen, OR², and optionally substituted C₁-C₆ aliphatic. In some embodiments, each R^{D} is independently selected from the group consisting of halogen, OR², optionally substituted C₁-C₃ alkyl, and optionally substituted C₂-C₃ alkenyl. In some embodiments, each R^{D} is independently selected from the group consisting of OR², optionally substituted C₁-C₃ alkyl, and optionally substituted C₂-C₃ alkenyl.

In some embodiments, each R^{D} is independently selected from halogen, —C(R^{d})₂OR²,

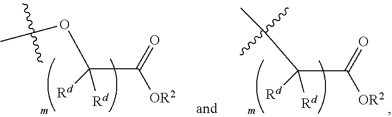

wherein
each R^{d} is independently hydrogen, optionally substituted methyl, —OH, —OMe, or —CD₃, wherein, two instances R^{d} may, with the atoms on which they are attached, form a cyclopropyl ring; and m is 0, 1, 2, or 3.

In some embodiments, r is 1 and R^{D} is —C(R^{d})₂OR² or

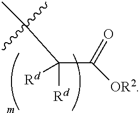

In some embodiments, r is 1 and R^{D} is —C(R^{d})₂OH or

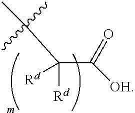

In some embodiments, R^{D} is selected from the group consisting of

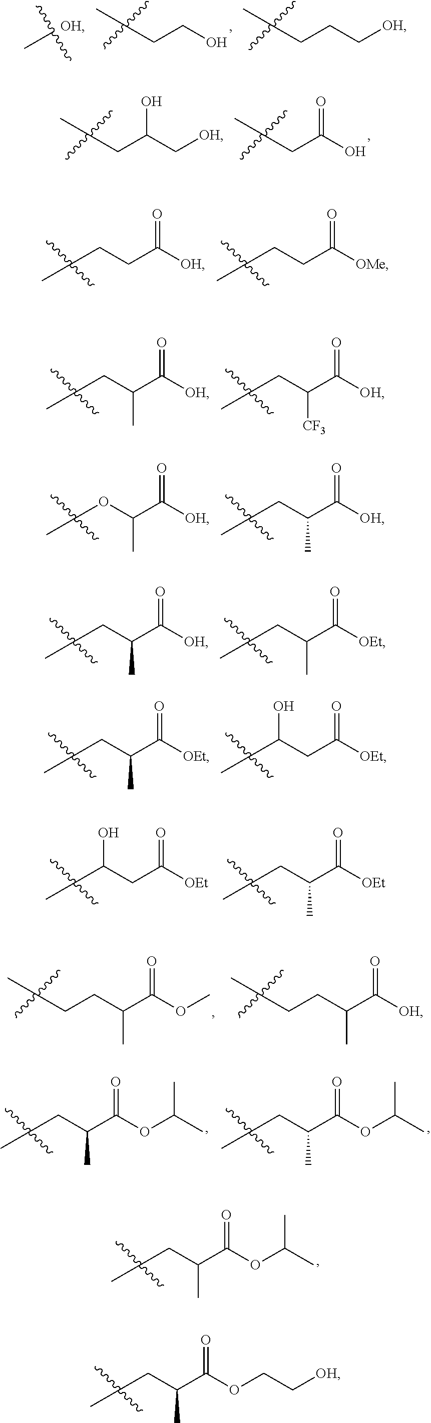

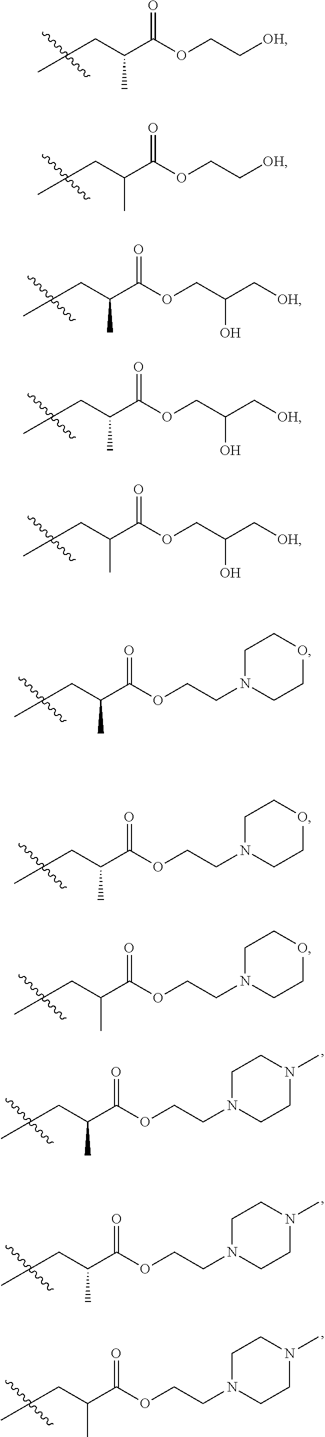

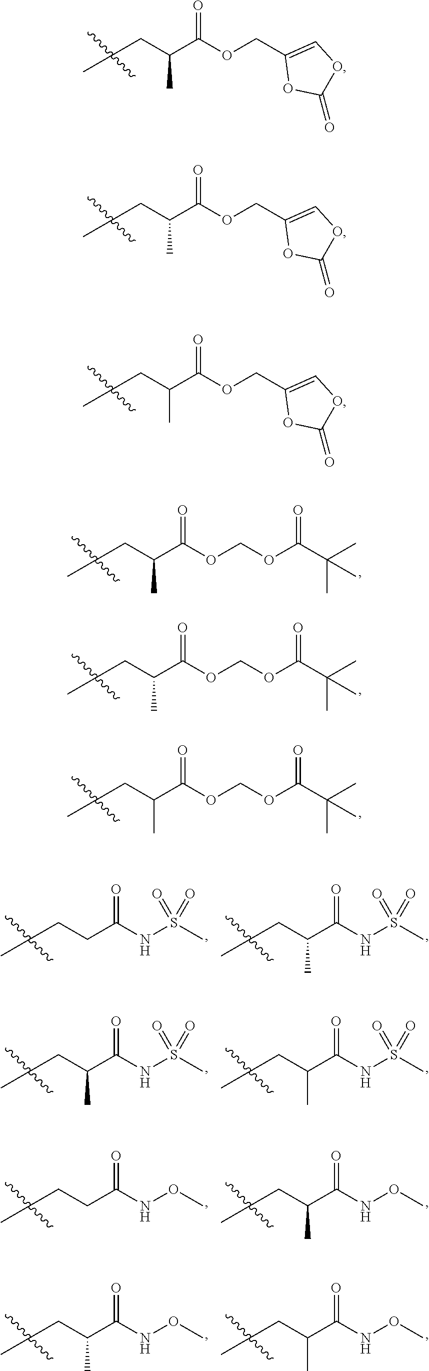

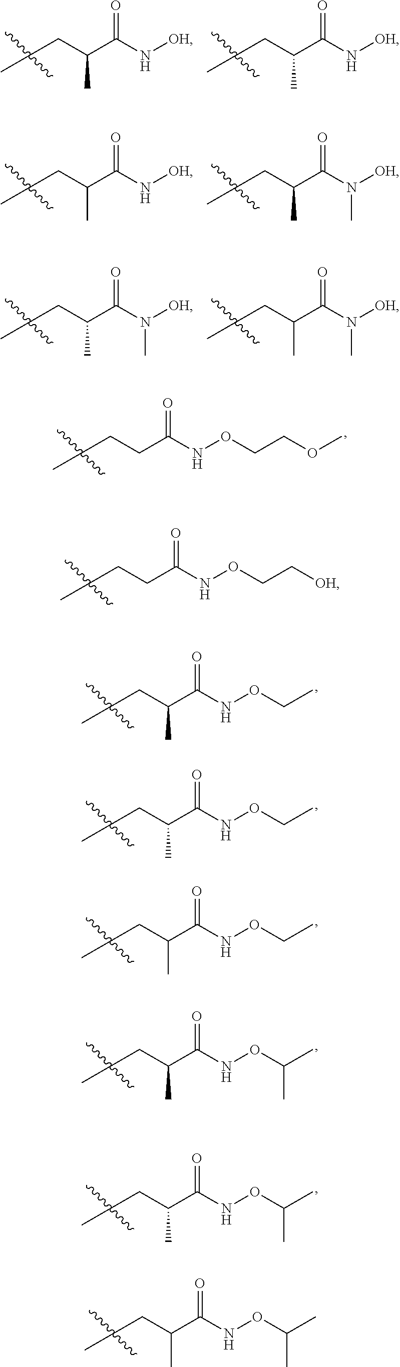

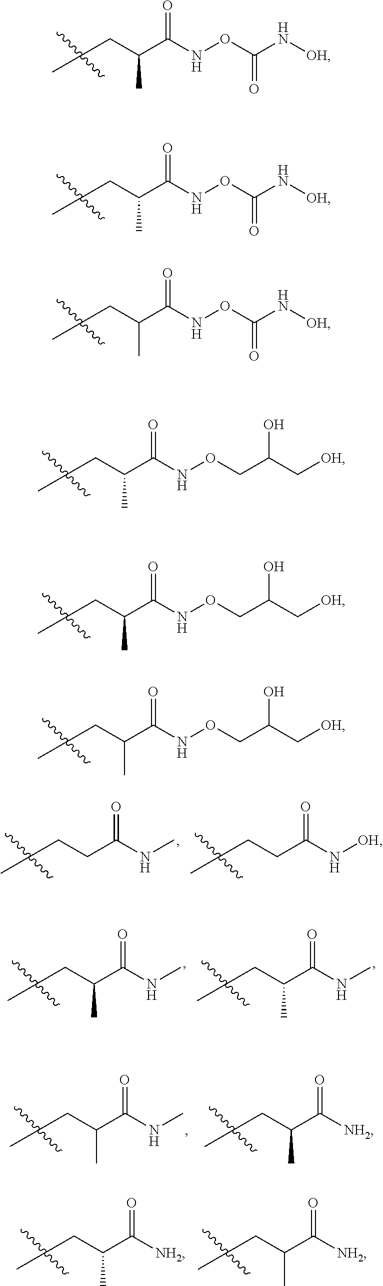

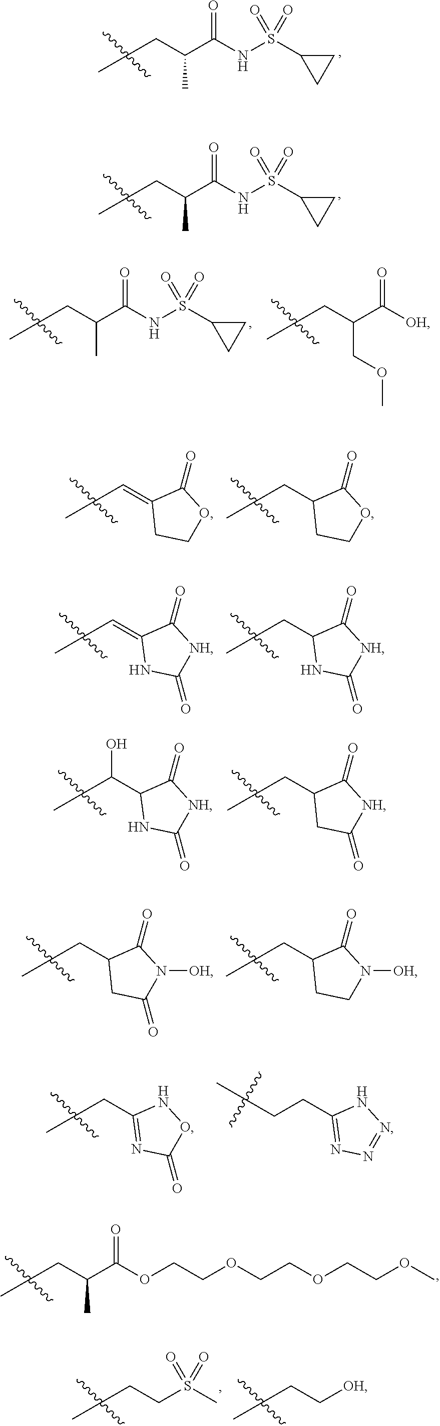

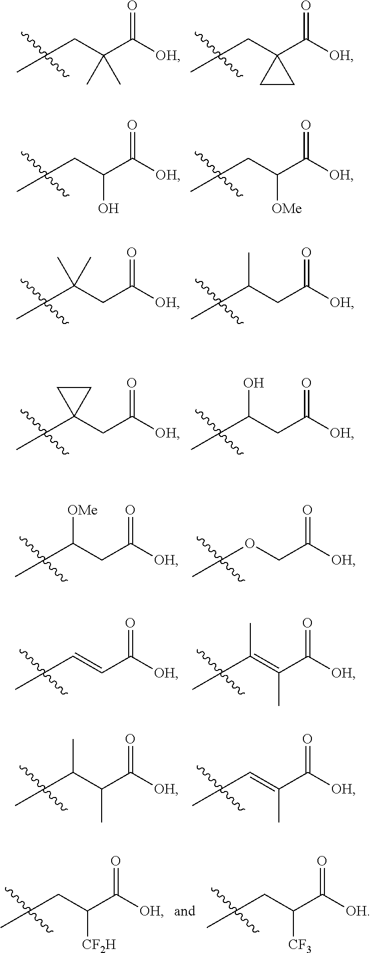

In some embodiments, R^{D} is selected from the group consisting of

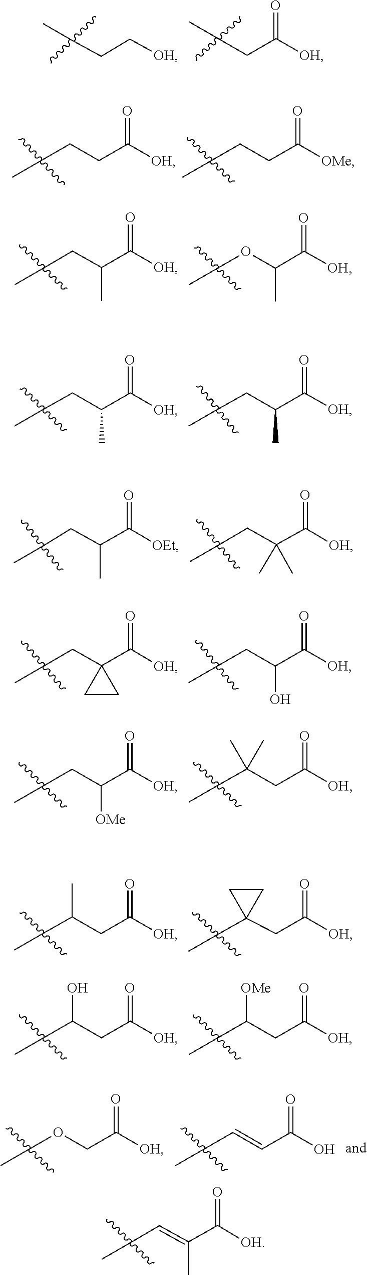

In some embodiments, R^{D} is selected from the group consisting of

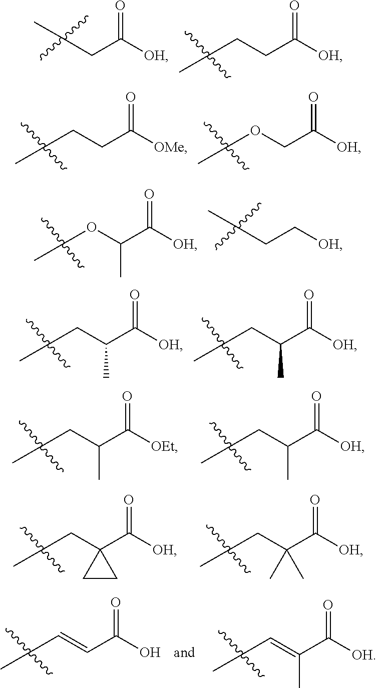

In some embodiments, R^{D} is selected from the group consisting of

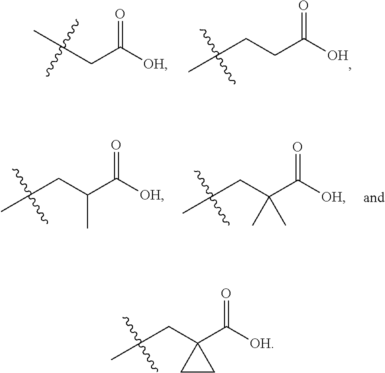

In some embodiments, R^{D} is

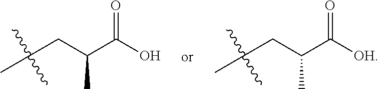

### R¹

In some embodiments, R¹ is selected from the group consisting of hydrogen, cyano, —OR², —(CH₂)₀₋₃N(R²)₂, optionally substituted C₁-C₃ aliphatic, 3-6-membered heterocyclyl comprising 1-3 heteroatoms selected from the group consisting of N, O or S, and —CD₃, In some embodiments, R¹ is selected from the group consisting of hydrogen, cyano, —OR², —(CH₂)₀₋₃N(R²)₂, optionally substituted C₁-C₃ aliphatic, and —CD₃. In some embodiments, R¹ is selected from the group consisting of hydrogen, cyano, and optionally substituted C₁-C₃ aliphatic. In some embodiments, R¹ is selected from the group consisting of hydrogen, cyano, optionally substituted methyl, and —CD₃. In some embodiments, R¹ is optionally substituted methyl. In some embodiments, R¹ is —CH₃. In some embodiments, R¹ is hydrogen. In some embodiments, R¹ is cyano. In some embodiments, R¹ is —CD₃. In some embodiments, R¹ is —CH₂NHCH₂CF₃. In some embodiments, R¹ is CH₂NH₂.

### R²

In some embodiments, each R² is independently selected from hydrogen, optionally substituted C₁-C₆ aliphatic, —OH, C₁-C₆ alkoxy, —S(O)₂(optionally substituted C₁-C₆ aliphatic). In some embodiments, each R² is independently hydrogen or optionally substituted C₁-C₆ aliphatic. In some embodiments, each R² is independently hydrogen or optionally substituted C₁-C₃ aliphatic. In some embodiments, each R² is independently hydrogen or optionally substituted methyl. In some embodiments, R² is optionally substituted C₁-C₆ aliphatic. In some embodiments, R² is hydrogen. In some embodiments, each R² is independently optionally substituted methyl or optionally substituted ethyl. In some embodiments, each R² is independently optionally substituted methyl.

### R^{d}

In some embodiments, each R^{d} is independently selected from the group consisting of hydrogen, —OH, —CD₃, —C(O)N(R²)₂, C(O)OR², —OR², —N(R²)₂, optionally substituted C₁-C₆ aliphatic, optionally substituted 5-6-membered heteroaryl, and optionally substituted 3-6-membered heterocyclyl comprising 1-3 heteroatoms selected from the group consisting of N, O or S. In some embodiments, each R^{d} is independently selected from the group consisting of hydrogen, optionally substituted C₁₋₃ alkyl, —OH, —OMe, or —CD₃, wherein, two instances R^{d} may, with the atoms on which they are attached, form a cyclopropyl ring. In some embodiments, each R^{d} is independently selected from the group consisting of hydrogen, methyl, —CF₃, —CF₂H, or —CFH₂. In some embodiments, each R^{d} is independently selected from hydrogen and methyl. In some embodiments, R^{d} is hydrogen.

### X

In some embodiments, X is selected from the group consisting of —O—, —S—, —CH₂—, —C(OH)H—, —SO—, —CO—, —SO₂—, —CFH—, —CF₂—, and —N(R²)—. In some embodiments, X is selected from the group consisting of —O—, —S—, —CH₂—, —SO—, —CO—, —C(OH)H, and —SO₂—. In some embodiments, X is —O—. In some embodiments, X is —S—. In some embodiments, X is —CH₂—. In some embodiments, X is —SO—. In some embodiments, X is —CO—. In some embodiments, X is —C(OH)H—. In some embodiments, X is —SO₂—. In some embodiments, X is

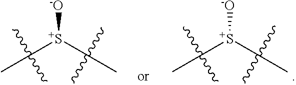

In some embodiments, X is

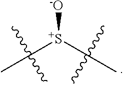

In some embodiments, X is

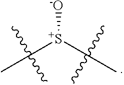

### Y

In some embodiments, Y is selected from the group consisting of —S(O)₂N(R²)—, —OC(O)N(R²)—, and —C(O)N(R²)—. In some embodiments, Y is selected from the group consisting of —S(O)₂N(H)—, —OC(O)N(H)—, and —C(O)N(H)—. In some embodiments, Y is —S(O)₂N(R²)—. In some embodiments, Y is —OC(O)N(R²)—. In some embodiments, Y is —C(O)N(R²)—. In some embodiments, Y is —S(O)₂N(Me)-. In some embodiments, Y is —OC(O)N(Me)-. In some embodiments, Y is —C(O)N(Me)-. In some embodiments, Y is —S(O)₂N(H)—. In some embodiments, Y is —OC(O)N(H)—. In some embodiments, Y is —C(O)N(H)—.

m, n, p, q, and r

In some embodiments, m is 0, 1, 2, or 3. In some embodiments, m is 1, 2, or 3. In some embodiments, m is 0. In some embodiments, m is 1. In some embodiments, m is 2. In some embodiments, m is 3.

In some embodiments, n is 0, 1, 2, or 3. In some embodiments, n is 1, 2, or 3. In some embodiments, n is 0. In some embodiments, n is 1. In some embodiments, n is 2. In some embodiments, n is 3.

In some embodiments, p is 0, 1, 2, 3, or 4. In some embodiments, p is 1, 2, 3, or 4. In some embodiments, p is 0. In some embodiments, p is 1. In some embodiments, p is 2. In some embodiments, p is 3. In some embodiments, p is 4.

In some embodiments, q is 0, 1, or 2. In some embodiments, q is 1 or 2. In some embodiments, q is 0. In some embodiments, q is 1. In some embodiments, q is 2.

In some embodiments, r is 0, 1, 2, 3, 4, or 5. In some embodiments, r is 1, 2, 3, or 4. In some embodiments, r is 0. In some embodiments, r is 1. In some embodiments, r is 2. In some embodiments, r is 3. In some embodiments, r is 4. In some embodiments, r is 5.

In some embodiments, the present disclosure includes compounds listed in Table 1.

TABLE 1

| Compound | |
|---|---|
| No. | Structure |
| 1 | 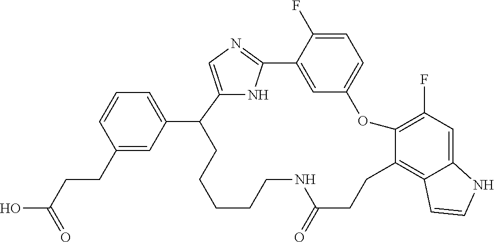 [SMILES: O=C(O)CCc1cccc(C2CCCCCNC(=O)CCc3c(c(F)cc4[nH]ccc34)Oc3ccc(F)c(c3)-c3ncc2[nH]3)c1] |
| 2 | 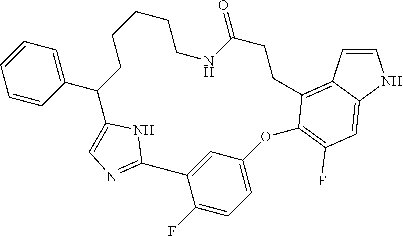 [SMILES: O=C1CCc2c(c(F)cc3[nH]ccc23)Oc2ccc(F)c(c2)-c2ncc([nH]2)C(c2ccccc2)CCCCCN1] |
| 3 | 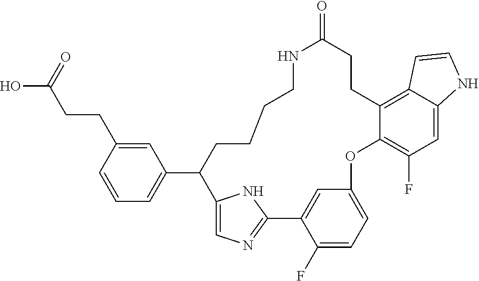 [SMILES: O=C(O)CCc1cccc(C2CCCCNC(=O)CCc3c(c(F)cc4[nH]ccc34)Oc3ccc(F)c(c3)-c3ncc2[nH]3)c1] |
| 4 | 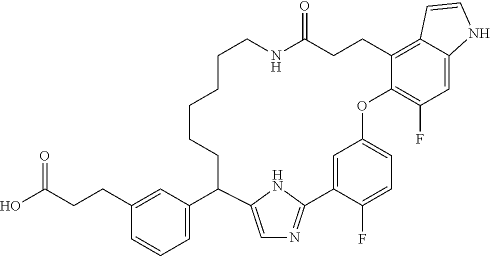 [SMILES: O=C(O)CCc1cccc(C2CCCCCCNC(=O)CCc3c(c(F)cc4[nH]ccc34)Oc3ccc(F)c(c3)-c3ncc2[nH]3)c1] |
| 5 | 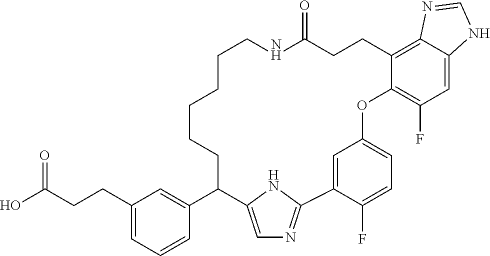 [SMILES: O=C(O)CCc1cccc(C2CCCCCCNC(=O)CCc3c(c(F)cc4[nH]cnc34)Oc3ccc(F)c(c3)-c3ncc2[nH]3)c1] |
| 6 | 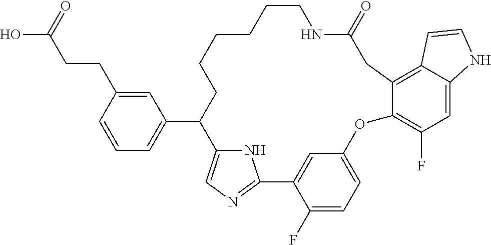 [SMILES: O=C(O)CCc1cccc(C2CCCCCCNC(=O)Cc3c(c(F)cc4[nH]ccc34)Oc3ccc(F)c(c3)-c3ncc2[nH]3)c1] |
| 7 | 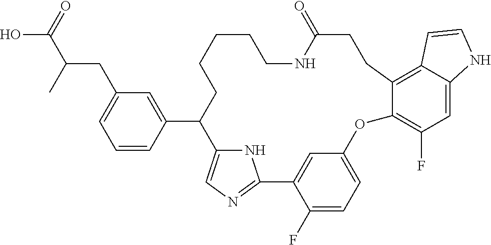 [SMILES: CC(Cc1cccc(C2CCCCCNC(=O)CCc3c(c(F)cc4[nH]ccc34)Oc3ccc(F)c(c3)-c3ncc2[nH]3)c1)C(=O)O] |
| 8 | 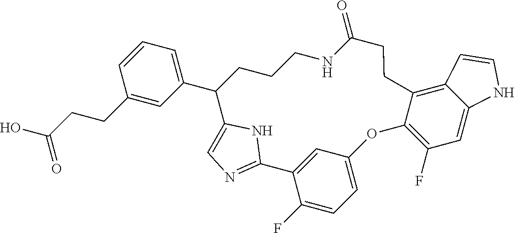 [SMILES: O=C(O)CCc1cccc(C2CCCNC(=O)CCc3c(c(F)cc4[nH]ccc34)Oc3ccc(F)c(c3)-c3ncc2[nH]3)c1] |
| 9 | 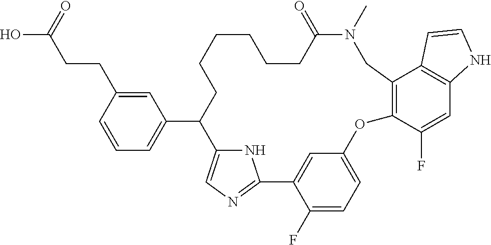 [SMILES: CN1Cc2c(c(F)cc3[nH]ccc23)Oc2ccc(F)c(c2)-c2ncc([nH]2)C(c2cccc(CCC(=O)O)c2)CCCCCCC1=O] |
| 10 | 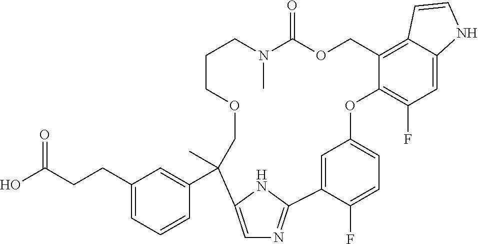 [SMILES: CN1CCCOCC(C)(c2cccc(CCC(=O)O)c2)c2cnc([nH]2)-c2cc(ccc2F)Oc2c(F)cc3[nH]ccc3c2COC1=O] |
| 11 | 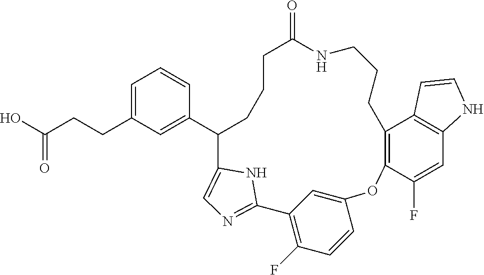 [SMILES: O=C(O)CCc1cccc(C2CCCC(=O)NCCCc3c(c(F)cc4[nH]ccc34)Oc3ccc(F)c(c3)-c3ncc2[nH]3)c1] |
| 12 | 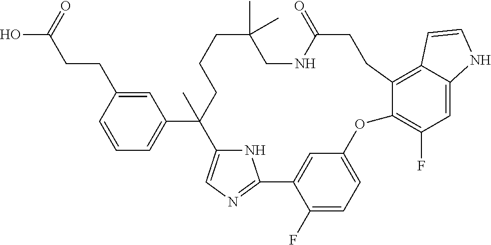 [SMILES: CC1(C)CCCC(C)(c2cccc(CCC(=O)O)c2)c2cnc([nH]2)-c2cc(ccc2F)Oc2c(F)cc3[nH]ccc3c2CCC(=O)NC1] |
| 13 | 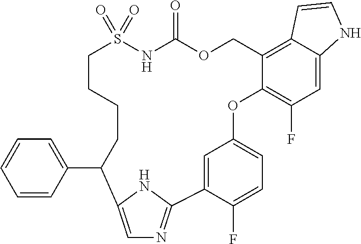 [SMILES: O=C1NS(=O)(=O)CCCCC(c2ccccc2)c2cnc([nH]2)-c2cc(ccc2F)Oc2c(F)cc3[nH]ccc3c2CO1] |
| 14 | 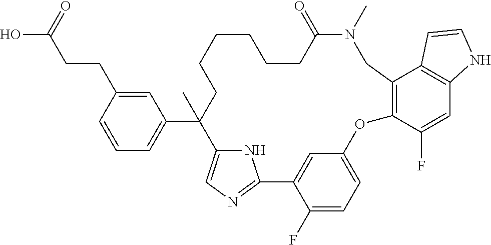 [SMILES: CN1Cc2c(c(F)cc3[nH]ccc23)Oc2ccc(F)c(c2)-c2ncc([nH]2)C(C)(c2cccc(CCC(=O)O)c2)CCCCCCC1=O] |
| 15 | 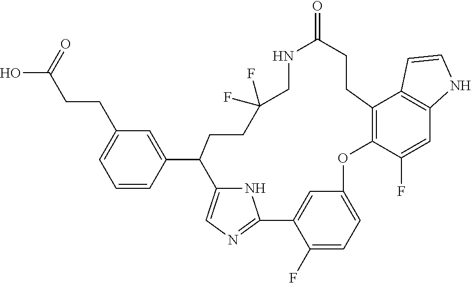 [SMILES: O=C(O)CCc1cccc(C2CCC(F)(F)CNC(=O)CCc3c(c(F)cc4[nH]ccc34)Oc3ccc(F)c(c3)-c3ncc2[nH]3)c1] |
| 16 | 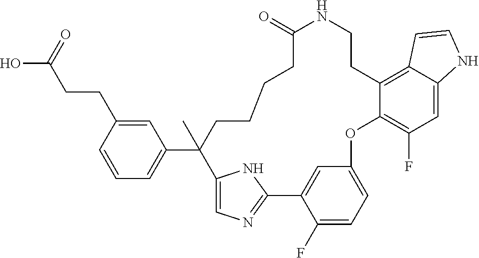 [SMILES: CC1(c2cccc(CCC(=O)O)c2)CCCCC(=O)NCCc2c(c(F)cc3[nH]ccc23)Oc2ccc(F)c(c2)-c2ncc1[nH]2] |
| 17 | 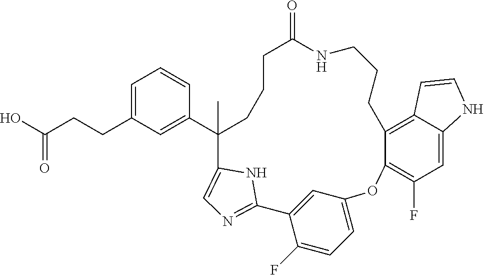 [SMILES: CC1(c2cccc(CCC(=O)O)c2)CCCC(=O)NCCCc2c(c(F)cc3[nH]ccc23)Oc2ccc(F)c(c2)-c2ncc1[nH]2] |
| 18 | 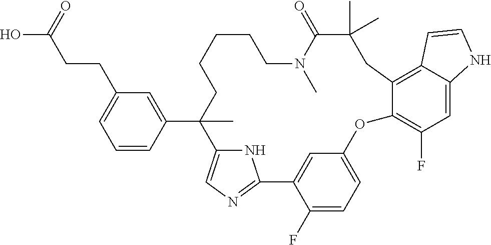 [SMILES: CN1CCCCCC(C)(c2cccc(CCC(=O)O)c2)c2cnc([nH]2)-c2cc(ccc2F)Oc2c(F)cc3[nH]ccc3c2CC(C)(C)C1=O] |
| 19 | 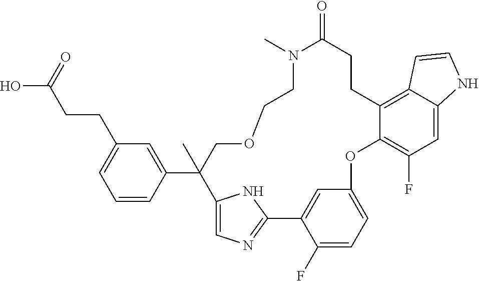 [SMILES: CN1CCOCC(C)(c2cccc(CCC(=O)O)c2)c2cnc([nH]2)-c2cc(ccc2F)Oc2c(F)cc3[nH]ccc3c2CCC1=O] |
| 20A | 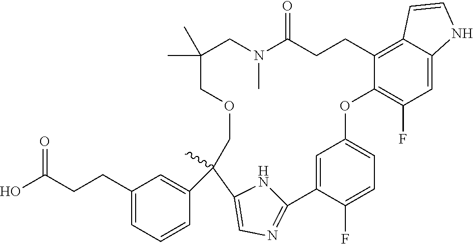 [SMILES: CN1CC(C)(C)COCC(C)(c2cccc(CCC(=O)O)c2)c2cnc([nH]2)-c2cc(ccc2F)Oc2c(F)cc3[nH]ccc3c2CCC1=O] |
| 20B | 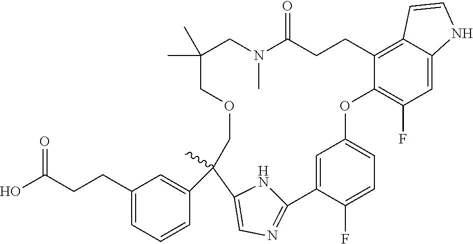 [SMILES: CN1CC(C)(C)COCC(C)(c2cccc(CCC(=O)O)c2)c2cnc([nH]2)-c2cc(ccc2F)Oc2c(F)cc3[nH]ccc3c2CCC1=O] |
| 20C | 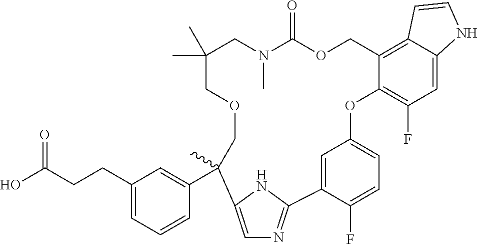 [SMILES: CN1CC(C)(C)COCC(C)(c2cccc(CCC(=O)O)c2)c2cnc([nH]2)-c2cc(ccc2F)Oc2c(F)cc3[nH]ccc3c2COC1=O] |
| 21 | 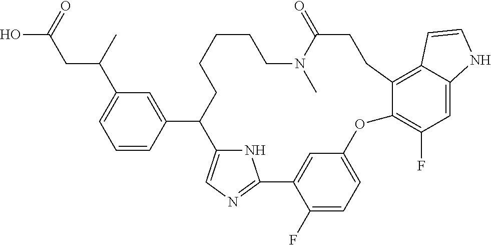 [SMILES: CC(CC(=O)O)c1cccc(C2CCCCCN(C)C(=O)CCc3c(c(F)cc4[nH]ccc34)Oc3ccc(F)c(c3)-c3ncc2[nH]3)c1] |
| 22 | 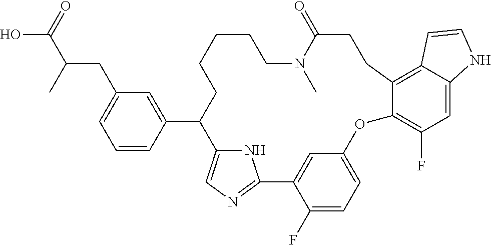 [SMILES: CC(Cc1cccc(C2CCCCCN(C)C(=O)CCc3c(c(F)cc4[nH]ccc34)Oc3ccc(F)c(c3)-c3ncc2[nH]3)c1)C(=O)O] |
| 23 | 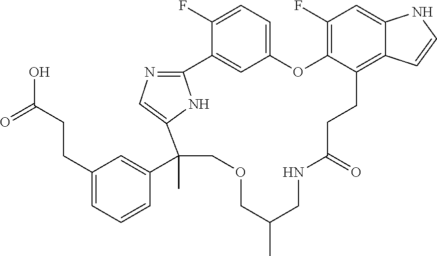 [SMILES: CC1CNC(=O)CCc2c(c(F)cc3[nH]ccc23)Oc2ccc(F)c(c2)-c2ncc([nH]2)C(C)(c2cccc(CCC(=O)O)c2)COC1] |
| 24 | 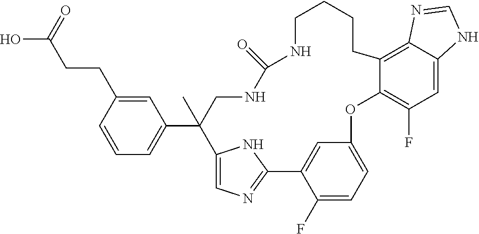 [SMILES: CC1(c2cccc(CCC(=O)O)c2)CNC(=O)NCCCCc2c(c(F)cc3[nH]cnc23)Oc2ccc(F)c(c2)-c2ncc1[nH]2] |
| 25 | 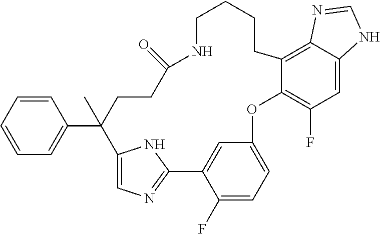 [SMILES: CC1(c2ccccc2)CCC(=O)NCCCCc2c(c(F)cc3[nH]cnc23)Oc2ccc(F)c(c2)-c2ncc1[nH]2] |
| 26 | 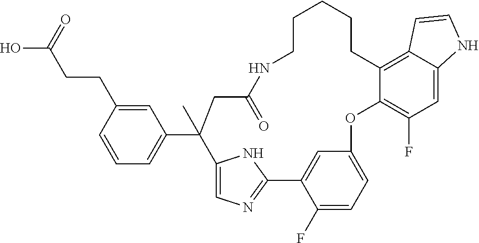 [SMILES: CC1(c2cccc(CCC(=O)O)c2)CC(=O)NCCCCCc2c(c(F)cc3[nH]ccc23)Oc2ccc(F)c(c2)-c2ncc1[nH]2] |
| 27 | 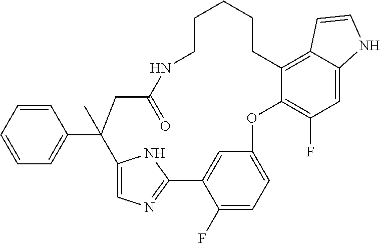 [SMILES: CC1(c2ccccc2)CC(=O)NCCCCCc2c(c(F)cc3[nH]ccc23)Oc2ccc(F)c(c2)-c2ncc1[nH]2] |
| 28 | 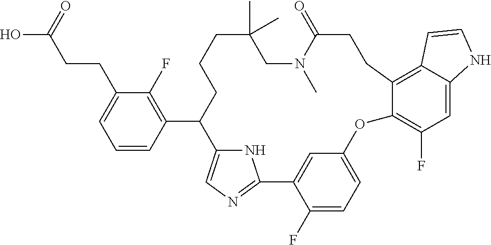 [SMILES: CN1CC(C)(C)CCCC(c2cccc(CCC(=O)O)c2F)c2cnc([nH]2)-c2cc(ccc2F)Oc2c(F)cc3[nH]ccc3c2CCC1=O] |
| 29 | 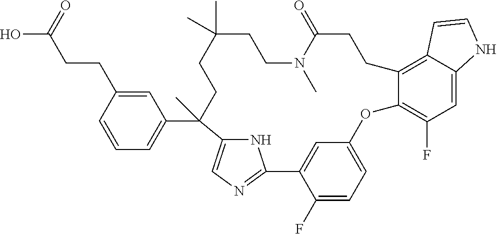 [SMILES: CN1CCC(C)(C)CCC(C)(c2cccc(CCC(=O)O)c2)c2cnc([nH]2)-c2cc(ccc2F)Oc2c(F)cc3[nH]ccc3c2CCC1=O] |
| 30 | 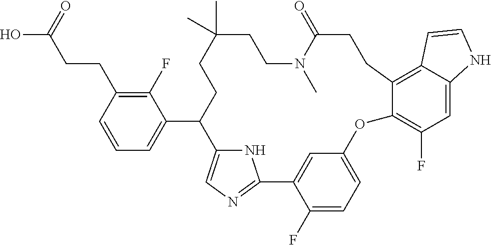 [SMILES: CN1CCC(C)(C)CCC(c2cccc(CCC(=O)O)c2F)c2cnc([nH]2)-c2cc(ccc2F)Oc2c(F)cc3[nH]ccc3c2CCC1=O] |
| 31 | 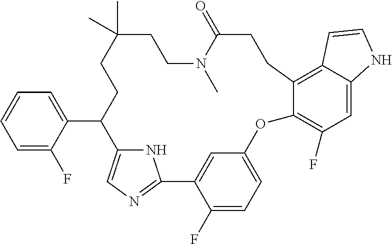 [SMILES: CN1CCC(C)(C)CCC(c2ccccc2F)c2cnc([nH]2)-c2cc(ccc2F)Oc2c(F)cc3[nH]ccc3c2CCC1=O] |
| 32 | 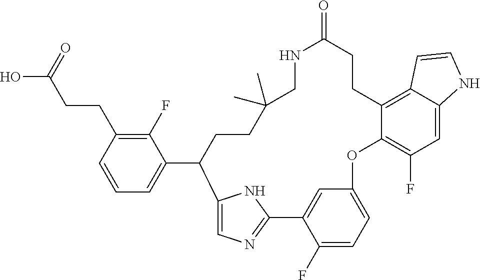 [SMILES: CC1(C)CCC(c2cccc(CCC(=O)O)c2F)c2cnc([nH]2)-c2cc(ccc2F)Oc2c(F)cc3[nH]ccc3c2CCC(=O)NC1] |
| 33 | 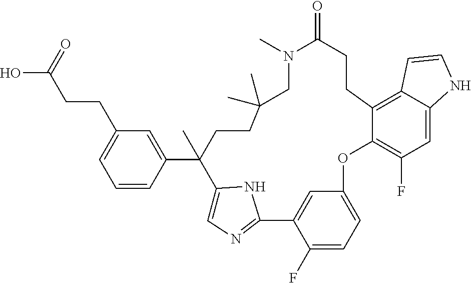 [SMILES: CN1CC(C)(C)CCC(C)(c2cccc(CCC(=O)O)c2)c2cnc([nH]2)-c2cc(ccc2F)Oc2c(F)cc3[nH]ccc3c2CCC1=O] |
| 34 | 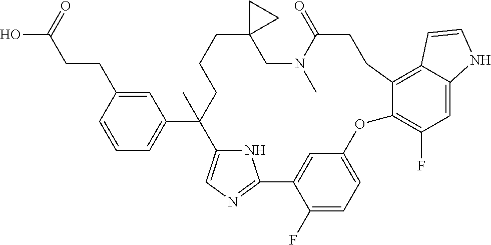 [SMILES: CN1CC2(CCCC(C)(c3cccc(CCC(=O)O)c3)c3cnc([nH]3)-c3cc(ccc3F)Oc3c(F)cc4[nH]ccc4c3CCC1=O)CC2] |
| 35 | 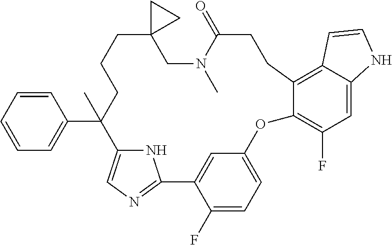 [SMILES: CN1CC2(CCCC(C)(c3ccccc3)c3cnc([nH]3)-c3cc(ccc3F)Oc3c(F)cc4[nH]ccc4c3CCC1=O)CC2] |
| 36 | 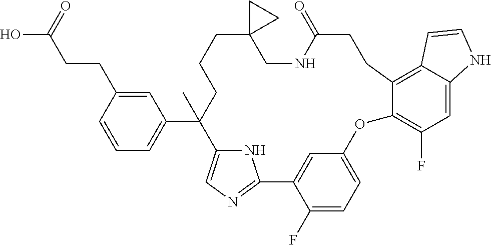 [SMILES: CC1(c2cccc(CCC(=O)O)c2)CCCC2(CC2)CNC(=O)CCc2c(c(F)cc3[nH]ccc23)Oc2ccc(F)c(c2)-c2ncc1[nH]2] |
| 37 | 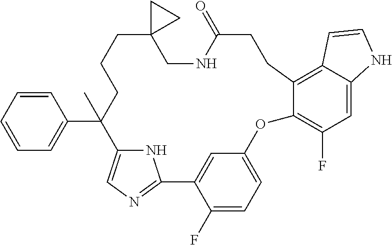 [SMILES: CC1(c2ccccc2)CCCC2(CC2)CNC(=O)CCc2c(c(F)cc3[nH]ccc23)Oc2ccc(F)c(c2)-c2ncc1[nH]2] |
| 38 | 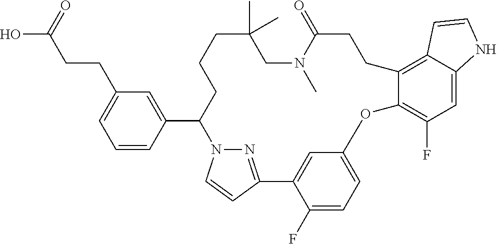 [SMILES: CN1CC(C)(C)CCCC(c2cccc(CCC(=O)O)c2)n2ccc(n2)-c2cc(ccc2F)Oc2c(F)cc3[nH]ccc3c2CCC1=O] |
| 39 | 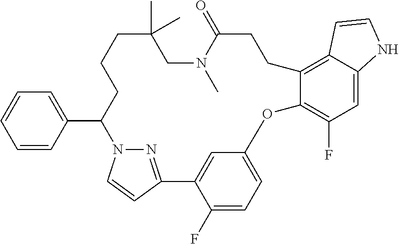 [SMILES: CN1CC(C)(C)CCCC(c2ccccc2)n2ccc(n2)-c2cc(ccc2F)Oc2c(F)cc3[nH]ccc3c2CCC1=O] |
| 40A | 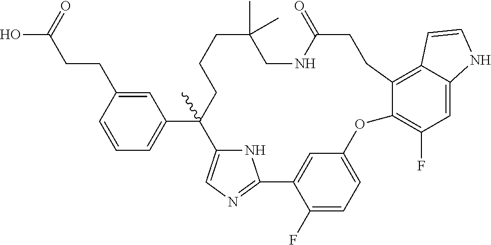 [SMILES: CC1(C)CCCC(C)(c2cccc(CCC(=O)O)c2)c2cnc([nH]2)-c2cc(ccc2F)Oc2c(F)cc3[nH]ccc3c2CCC(=O)NC1] |
| 40B | 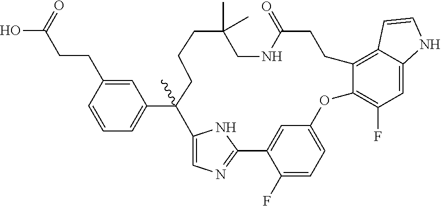 [SMILES: CC1(C)CCCC(C)(c2cccc(CCC(=O)O)c2)c2cnc([nH]2)-c2cc(ccc2F)Oc2c(F)cc3[nH]ccc3c2CCC(=O)NC1] |
| 41 | 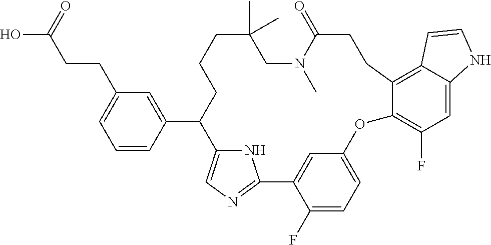 [SMILES: CN1CC(C)(C)CCCC(c2cccc(CCC(=O)O)c2)c2cnc([nH]2)-c2cc(ccc2F)Oc2c(F)cc3[nH]ccc3c2CCC1=O] |
| 42A | 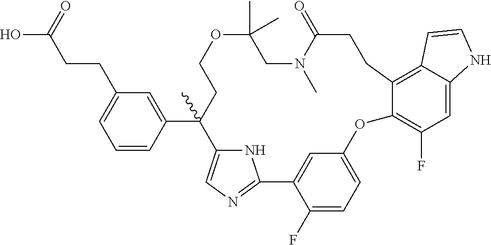 [SMILES: CN1CC(C)(C)OCCC(C)(c2cccc(CCC(=O)O)c2)c2cnc([nH]2)-c2cc(ccc2F)Oc2c(F)cc3[nH]ccc3c2CCC1=O] |
| 42A | 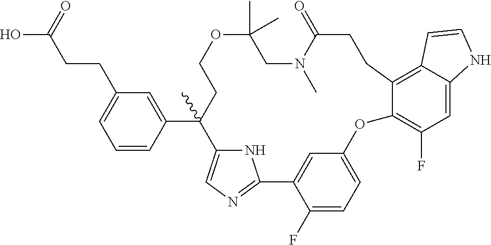 [SMILES: CN1CC(C)(C)OCCC(C)(c2cccc(CCC(=O)O)c2)c2cnc([nH]2)-c2cc(ccc2F)Oc2c(F)cc3[nH]ccc3c2CCC1=O] |
| 43 | 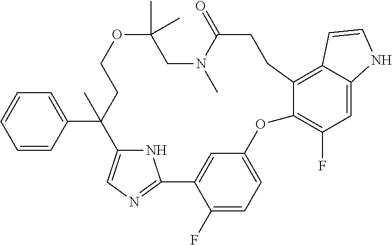 [SMILES: CN1CC(C)(C)OCCC(C)(c2ccccc2)c2cnc([nH]2)-c2cc(ccc2F)Oc2c(F)cc3[nH]ccc3c2CCC1=O] |
| 44 | 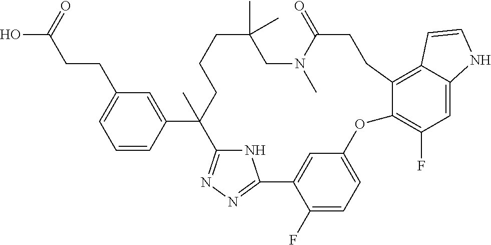 [SMILES: CN1CC(C)(C)CCCC(C)(c2cccc(CCC(=O)O)c2)c2nnc([nH]2)-c2cc(ccc2F)Oc2c(F)cc3[nH]ccc3c2CCC1=O] |
| 45 | 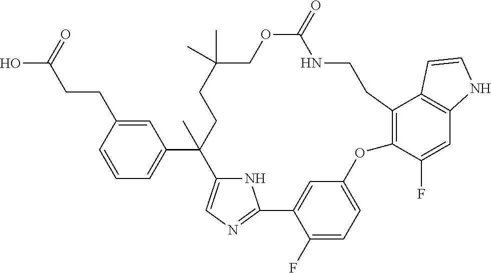 [SMILES: CC1(C)CCC(C)(c2cccc(CCC(=O)O)c2)c2cnc([nH]2)-c2cc(ccc2F)Oc2c(F)cc3[nH]ccc3c2CCNC(=O)OC1] |
| 46A | 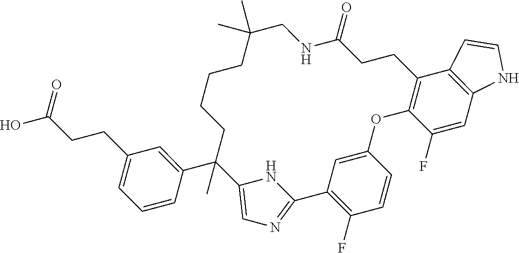 [SMILES: CC1(C)CCCCC(C)(c2cccc(CCC(=O)O)c2)c2cnc([nH]2)-c2cc(ccc2F)Oc2c(F)cc3[nH]ccc3c2CCC(=O)NC1] |
| 46B | 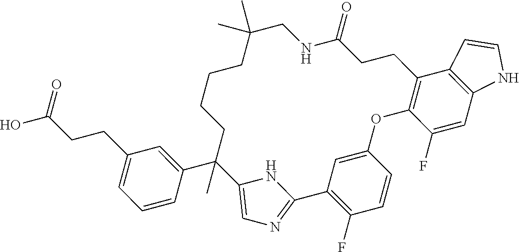 [SMILES: CC1(C)CCCCC(C)(c2cccc(CCC(=O)O)c2)c2cnc([nH]2)-c2cc(ccc2F)Oc2c(F)cc3[nH]ccc3c2CCC(=O)NC1] |
| 47A | 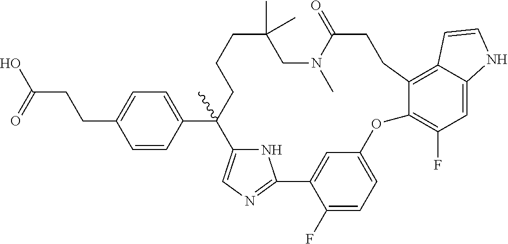 [SMILES: CN1CC(C)(C)CCCC(C)(c2ccc(CCC(=O)O)cc2)c2cnc([nH]2)-c2cc(ccc2F)Oc2c(F)cc3[nH]ccc3c2CCC1=O] |
| 47B | 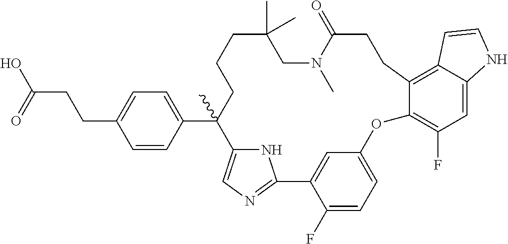 [SMILES: CN1CC(C)(C)CCCC(C)(c2ccc(CCC(=O)O)cc2)c2cnc([nH]2)-c2cc(ccc2F)Oc2c(F)cc3[nH]ccc3c2CCC1=O] |
| 48A | 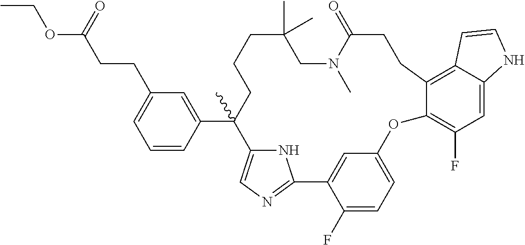 [SMILES: CCOC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 48B | 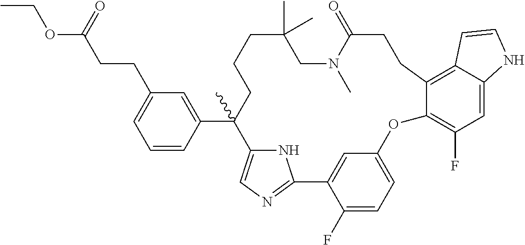 [SMILES: CCOC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 49A | 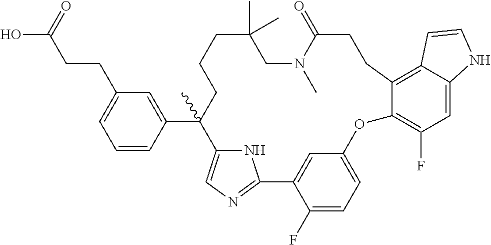 [SMILES: CN1CC(C)(C)CCCC(C)(c2cccc(CCC(=O)O)c2)c2cnc([nH]2)-c2cc(ccc2F)Oc2c(F)cc3[nH]ccc3c2CCC1=O] |
| 49B | 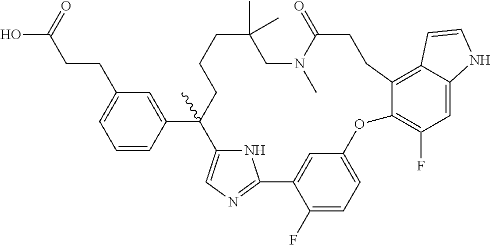 [SMILES: CN1CC(C)(C)CCCC(C)(c2cccc(CCC(=O)O)c2)c2cnc([nH]2)-c2cc(ccc2F)Oc2c(F)cc3[nH]ccc3c2CCC1=O] |
| 5250 | 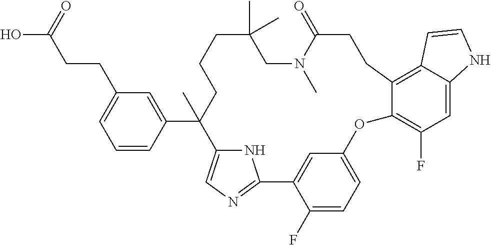 [SMILES: CN1CC(C)(C)CCCC(C)(c2cccc(CCC(=O)O)c2)c2cnc([nH]2)-c2cc(ccc2F)Oc2c(F)cc3[nH]ccc3c2CCC1=O] |
| 5251 | 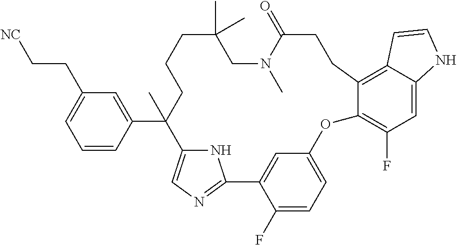 [SMILES: CN1CC(C)(C)CCCC(C)(c2cccc(CCC#N)c2)c2cnc([nH]2)-c2cc(ccc2F)Oc2c(F)cc3[nH]ccc3c2CCC1=O] |
| 52 | 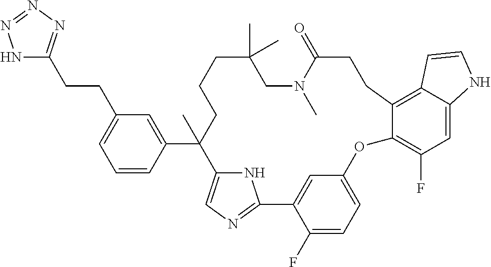 [SMILES: CN1CC(C)(C)CCCC(C)(c2cccc(CCc3nnn[nH]3)c2)c2cnc([nH]2)-c2cc(ccc2F)Oc2c(F)cc3[nH]ccc3c2CCC1=O] |
| 53A | 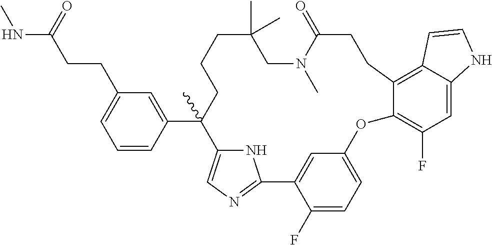 [SMILES: CNC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 53B | 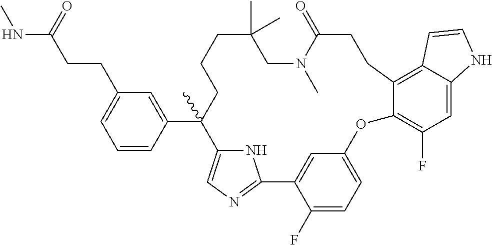 [SMILES: CNC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 54 | 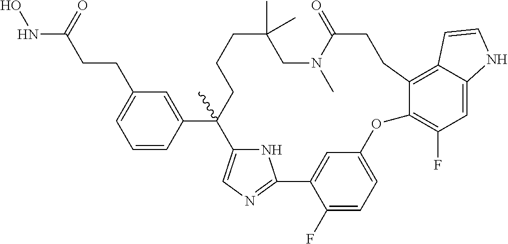 [SMILES: CN1CC(C)(C)CCCC(C)(c2cccc(CCC(=O)NO)c2)c2cnc([nH]2)-c2cc(ccc2F)Oc2c(F)cc3[nH]ccc3c2CCC1=O] |
| 55 | 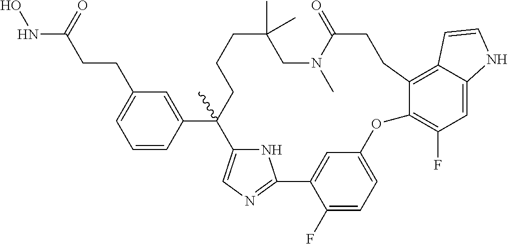 [SMILES: CN1CC(C)(C)CCCC(C)(c2cccc(CCC(=O)NO)c2)c2cnc([nH]2)-c2cc(ccc2F)Oc2c(F)cc3[nH]ccc3c2CCC1=O] |
| 56 | 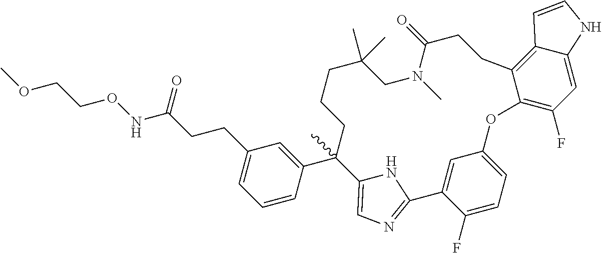 [SMILES: COCCONC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 57 | 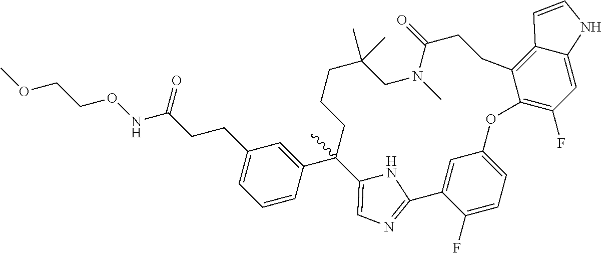 [SMILES: COCCONC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 58 | 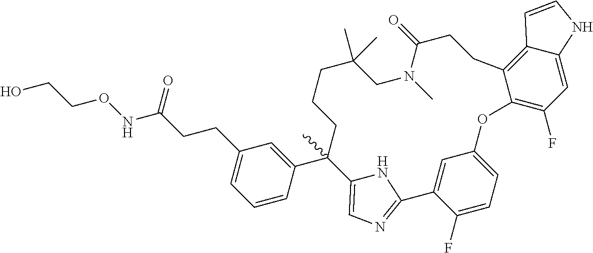 [SMILES: CN1CC(C)(C)CCCC(C)(c2cccc(CCC(=O)NOCCO)c2)c2cnc([nH]2)-c2cc(ccc2F)Oc2c(F)cc3[nH]ccc3c2CCC1=O] |
| 59 | 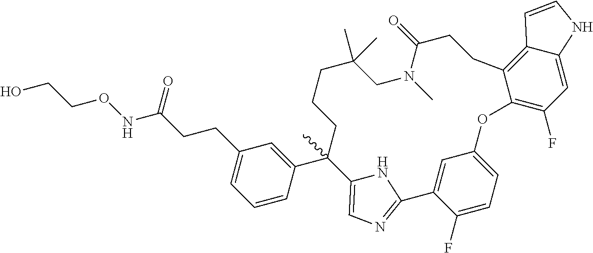 [SMILES: CN1CC(C)(C)CCCC(C)(c2cccc(CCC(=O)NOCCO)c2)c2cnc([nH]2)-c2cc(ccc2F)Oc2c(F)cc3[nH]ccc3c2CCC1=O] |
| 60A | 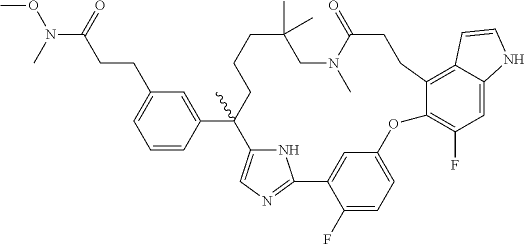 [SMILES: CON(C)C(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 60B | 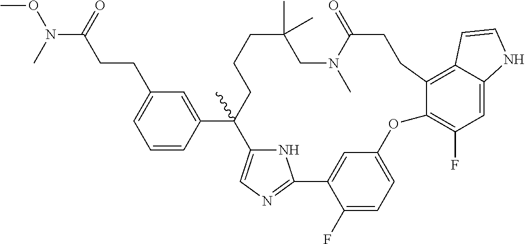 [SMILES: CON(C)C(=O)CCc1cccc(C2(C)CCCC(C)(C)CN(C)C(=O)CCc3c(c(F)cc4[nH]ccc34)Oc3ccc(F)c(c3)-c3ncc2[nH]3)c1] |
| 61 | 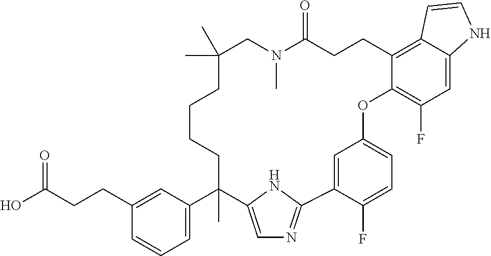 [SMILES: CN1CC(C)(C)CCCCC(C)(c2cccc(CCC(=O)O)c2)c2cnc([nH]2)-c2cc(ccc2F)Oc2c(F)cc3[nH]ccc3c2CCC1=O] |
| 62 | 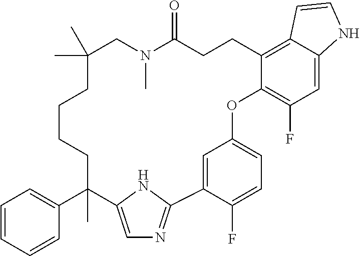 [SMILES: CN1CC(C)(C)CCCCC(C)(c2ccccc2)c2cnc([nH]2)-c2cc(ccc2F)Oc2c(F)cc3[nH]ccc3c2CCC1=O] |
| 63 | 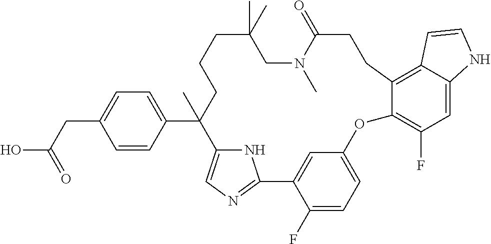 [SMILES: CN1CC(C)(C)CCCC(C)(c2ccc(CC(=O)O)cc2)c2cnc([nH]2)-c2cc(ccc2F)Oc2c(F)cc3[nH]ccc3c2CCC1=O] |
| 64A | 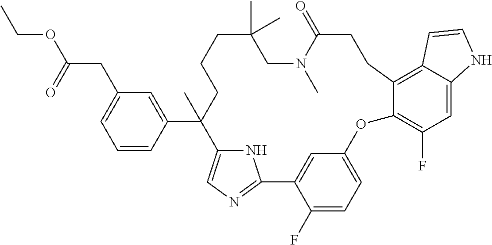 [SMILES: CCOC(=O)Cc1cccc(C2(C)CCCC(C)(C)CN(C)C(=O)CCc3c(c(F)cc4[nH]ccc34)Oc3ccc(F)c(c3)-c3ncc2[nH]3)c1] |
| 64B | 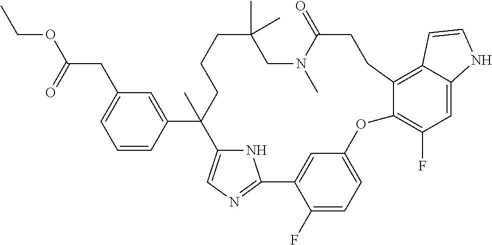 [SMILES: CCOC(=O)Cc1cccc(C2(C)CCCC(C)(C)CN(C)C(=O)CCc3c(c(F)cc4[nH]ccc34)Oc3ccc(F)c(c3)-c3ncc2[nH]3)c1] |
| 65A | 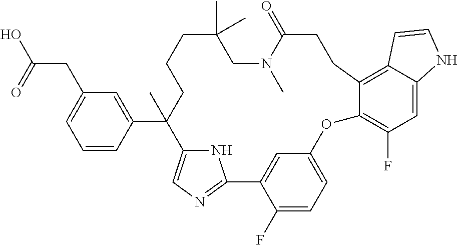 [SMILES: CN1CC(C)(C)CCCC(C)(c2cccc(CC(=O)O)c2)c2cnc([nH]2)-c2cc(ccc2F)Oc2c(F)cc3[nH]ccc3c2CCC1=O] |
| 65B | 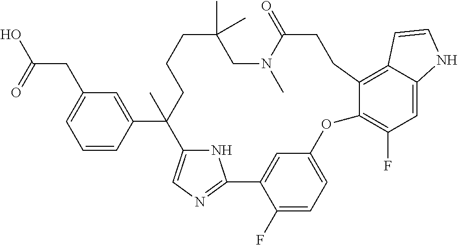 [SMILES: CN1CC(C)(C)CCCC(C)(c2cccc(CC(=O)O)c2)c2cnc([nH]2)-c2cc(ccc2F)Oc2c(F)cc3[nH]ccc3c2CCC1=O] |
| 66A | 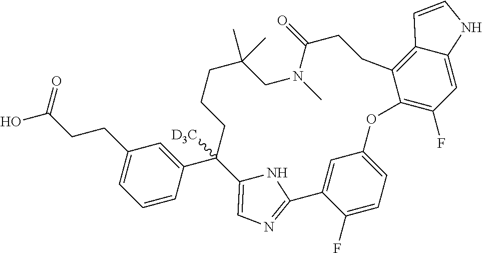 [SMILES: [2H]C([2H])([2H])C1(c2cccc(CCC(=O)O)c2)CCCC(C)(C)CN(C)C(=O)CCc2c(c(F)cc3[nH]ccc23)Oc2ccc(F)c(c2)-c2ncc1[nH]2] |
| 66B | 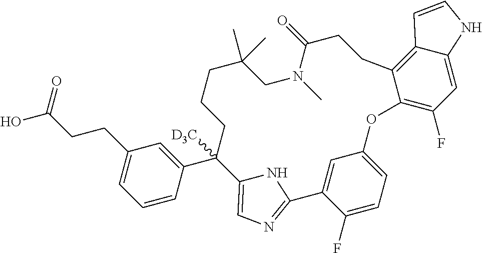 [SMILES: [2H]C([2H])([2H])C1(c2cccc(CCC(=O)O)c2)CCCC(C)(C)CN(C)C(=O)CCc2c(c(F)cc3[nH]ccc23)Oc2ccc(F)c(c2)-c2ncc1[nH]2] |
| 67A | 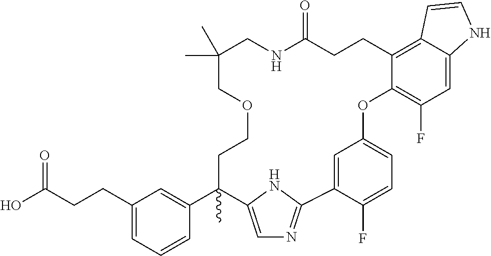 [SMILES: CC1(C)CNC(=O)CCc2c(c(F)cc3[nH]ccc23)Oc2ccc(F)c(c2)-c2ncc([nH]2)C(C)(c2cccc(CCC(=O)O)c2)CCOC1] |
| 67B | 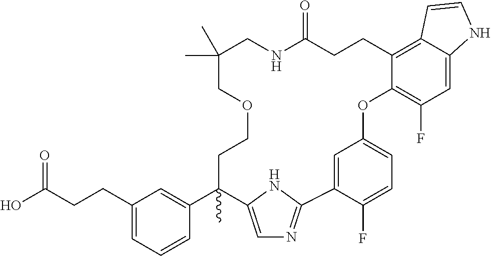 [SMILES: CC1(C)CNC(=O)CCc2c(c(F)cc3[nH]ccc23)Oc2ccc(F)c(c2)-c2ncc([nH]2)C(C)(c2cccc(CCC(=O)O)c2)CCOC1] |
| 68 | 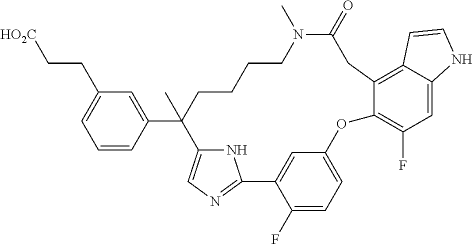 [SMILES: CN1CCCCC(C)(c2cccc(CCC(=O)O)c2)c2cnc([nH]2)-c2cc(ccc2F)Oc2c(F)cc3[nH]ccc3c2CC1=O] |
| 69A | 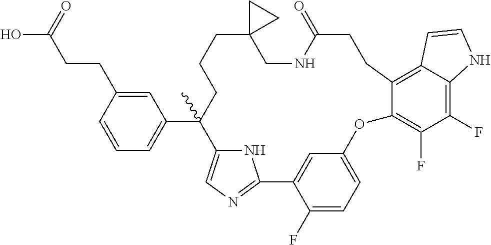 [SMILES: CC1(c2cccc(CCC(=O)O)c2)CCCC2(CC2)CNC(=O)CCc2c(c(F)c(F)c3[nH]ccc23)Oc2ccc(F)c(c2)-c2ncc1[nH]2] |
| 69B | 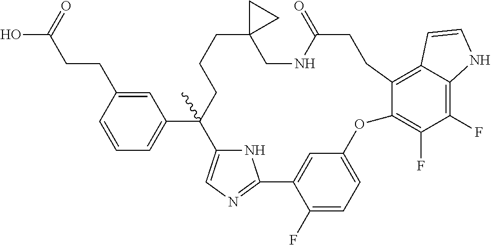 [SMILES: CC1(c2cccc(CCC(=O)O)c2)CCCC2(CC2)CNC(=O)CCc2c(c(F)c(F)c3[nH]ccc23)Oc2ccc(F)c(c2)-c2ncc1[nH]2] |
| 70 | 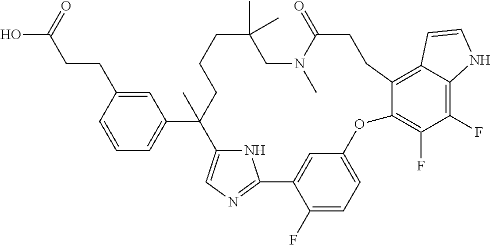 [SMILES: CN1CC(C)(C)CCCC(C)(c2cccc(CCC(=O)O)c2)c2cnc([nH]2)-c2cc(ccc2F)Oc2c(F)c(F)c3[nH]ccc3c2CCC1=O] |
| 71 | 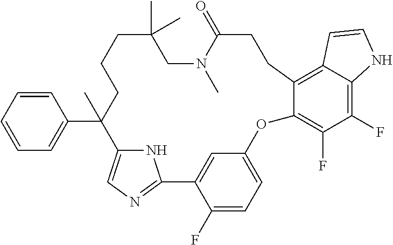 [SMILES: CN1CC(C)(C)CCCC(C)(c2ccccc2)c2cnc([nH]2)-c2cc(ccc2F)Oc2c(F)c(F)c3[nH]ccc3c2CCC1=O] |
| 72A | 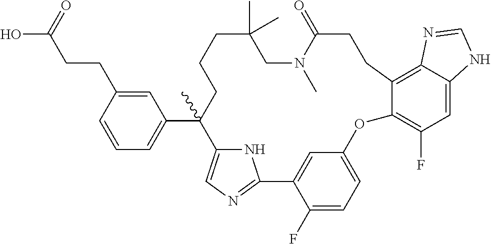 [SMILES: CN1CC(C)(C)CCCC(C)(c2cccc(CCC(=O)O)c2)c2cnc([nH]2)-c2cc(ccc2F)Oc2c(F)cc3[nH]cnc3c2CCC1=O] |
| 72B | 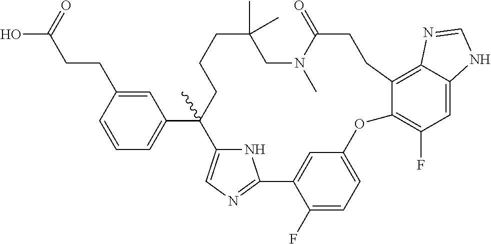 [SMILES: CN1CC(C)(C)CCCC(C)(c2cccc(CCC(=O)O)c2)c2cnc([nH]2)-c2cc(ccc2F)Oc2c(F)cc3[nH]cnc3c2CCC1=O] |
| 73 | 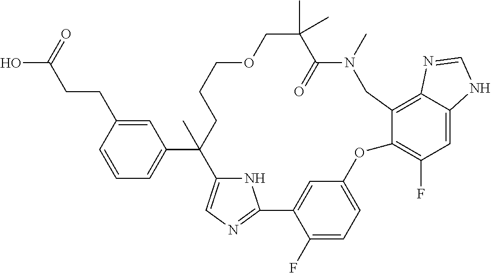 [SMILES: CN1Cc2c(c(F)cc3[nH]cnc23)Oc2ccc(F)c(c2)-c2ncc([nH]2)C(C)(c2cccc(CCC(=O)O)c2)CCCOCC(C)(C)C1=O] |
| 74 | 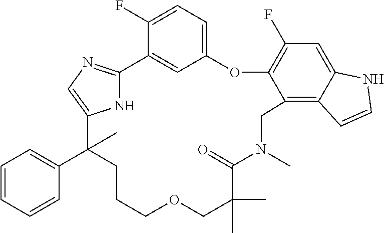 [SMILES: CN1Cc2c(c(F)cc3[nH]ccc23)Oc2ccc(F)c(c2)-c2ncc([nH]2)C(C)(c2ccccc2)CCCOCC(C)(C)C1=O] |
| 75A | 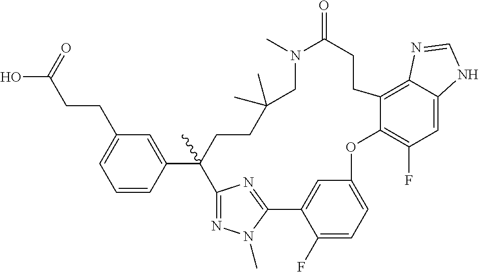 [SMILES: CN1CC(C)(C)CCC(C)(c2cccc(CCC(=O)O)c2)c2nc(n(C)n2)-c2cc(ccc2F)Oc2c(F)cc3[nH]cnc3c2CCC1=O] |
| 75B | 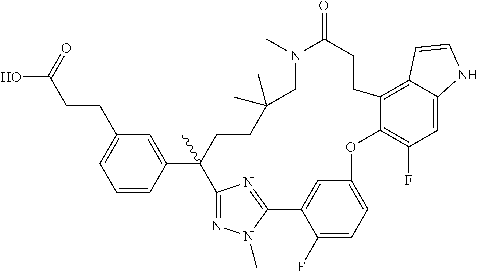 [SMILES: CN1CC(C)(C)CCC(C)(c2cccc(CCC(=O)O)c2)c2nc(n(C)n2)-c2cc(ccc2F)Oc2c(F)cc3[nH]ccc3c2CCC1=O] |
| 76A | 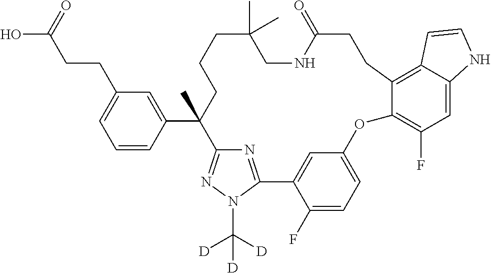 [SMILES: [2H]C([2H])([2H])n1nc2nc1-c1cc(ccc1F)Oc1c(F)cc3[nH]ccc3c1CCC(=O)NCC(C)(C)CCC[C@@]2(C)c1cccc(CCC(=O)O)c1] |
| 76B | 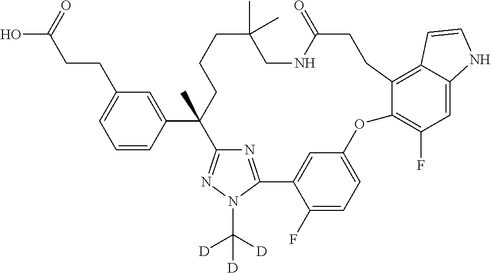 [SMILES: [2H]C([2H])([2H])n1nc2nc1-c1cc(ccc1F)Oc1c(F)cc3[nH]ccc3c1CCC(=O)NCC(C)(C)CCC[C@@]2(C)c1cccc(CCC(=O)O)c1] |
| 77A | 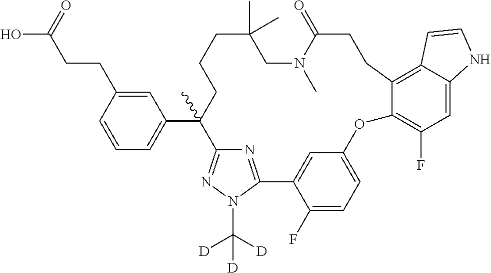 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)O)c1] |
| 77B | 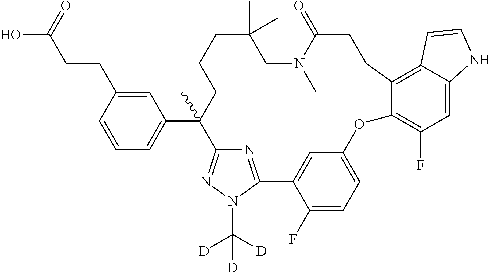 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)O)c1] |
| 78 | 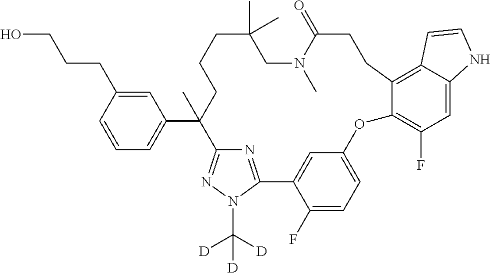 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCCO)c1] |
| 79A | 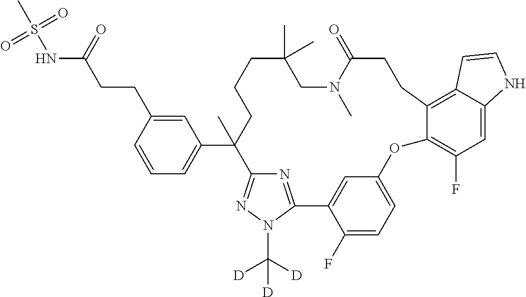 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)NS(C)(=O)=O)c1] |
| 79B | 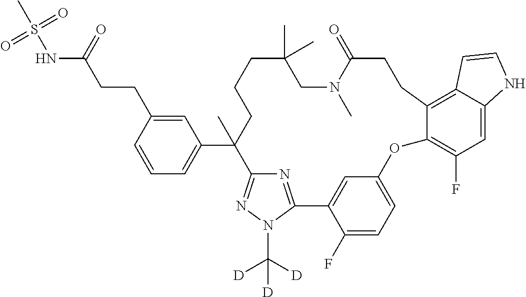 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)NS(C)(=O)=O)c1] |
| 80 | 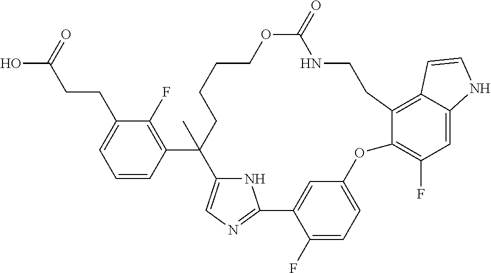 [SMILES: CC1(c2cccc(CCC(=O)O)c2F)CCCCOC(=O)NCCc2c(c(F)cc3[nH]ccc23)Oc2ccc(F)c(c2)-c2ncc1[nH]2] |
| 81 | 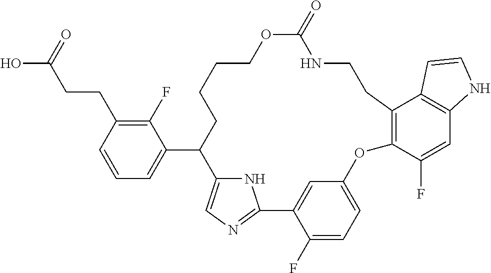 [SMILES: O=C(O)CCc1cccc(C2CCCCOC(=O)NCCc3c(c(F)cc4[nH]ccc34)Oc3ccc(F)c(c3)-c3ncc2[nH]3)c1F] |
| 82 | 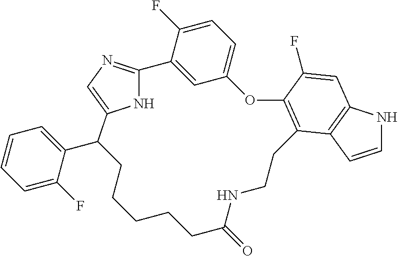 [SMILES: O=C1CCCCCC(c2ccccc2F)c2cnc([nH]2)-c2cc(ccc2F)Oc2c(F)cc3[nH]ccc3c2CCN1] |
| 83 | 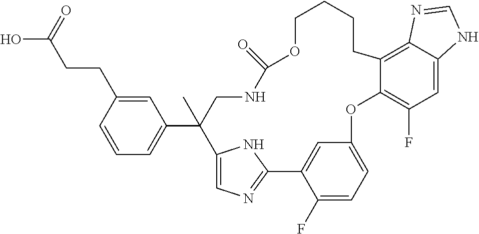 [SMILES: CC1(c2cccc(CCC(=O)O)c2)CNC(=O)OCCCCc2c(c(F)cc3[nH]cnc23)Oc2ccc(F)c(c2)-c2ncc1[nH]2] |
| 84 | 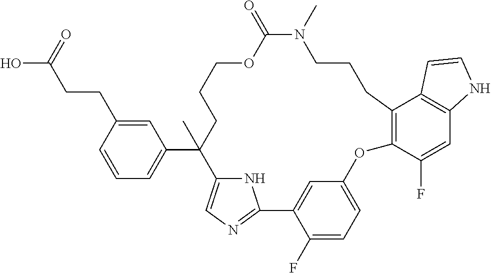 [SMILES: CN1CCCc2c(c(F)cc3[nH]ccc23)Oc2ccc(F)c(c2)-c2ncc([nH]2)C(C)(c2cccc(CCC(=O)O)c2)CCCOC1=O] |
| 85 | 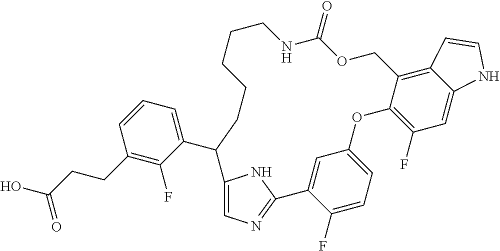 [SMILES: O=C(O)CCc1cccc(C2CCCCCNC(=O)OCc3c(c(F)cc4[nH]ccc34)Oc3ccc(F)c(c3)-c3ncc2[nH]3)c1F] |
| 86 | 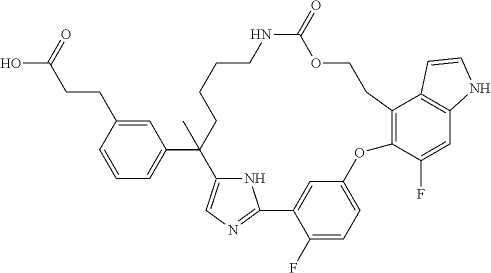 [SMILES: CC1(c2cccc(CCC(=O)O)c2)CCCCNC(=O)OCCc2c(c(F)cc3[nH]ccc23)Oc2ccc(F)c(c2)-c2ncc1[nH]2] |
| 87 | 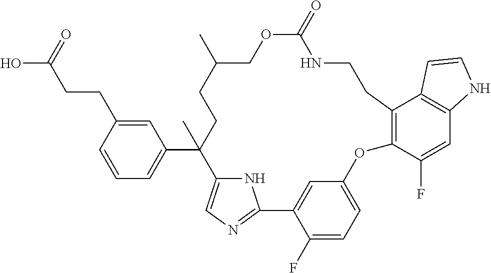 [SMILES: CC1CCC(C)(c2cccc(CCC(=O)O)c2)c2cnc([nH]2)-c2cc(ccc2F)Oc2c(F)cc3[nH]ccc3c2CCNC(=O)OC1] |
| 88 | 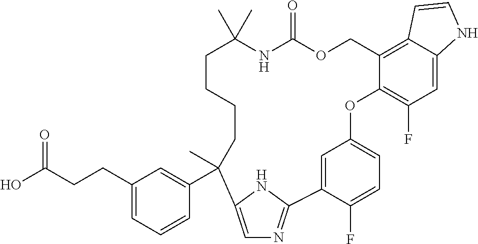 [SMILES: CC1(C)CCCCC(C)(c2cccc(CCC(=O)O)c2)c2cnc([nH]2)-c2cc(ccc2F)Oc2c(F)cc3[nH]ccc3c2COC(=O)N1] |
| 89 | 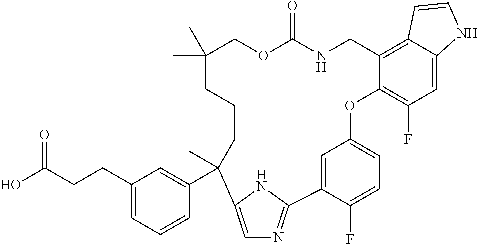 [SMILES: CC1(C)CCCC(C)(c2cccc(CCC(=O)O)c2)c2cnc([nH]2)-c2cc(ccc2F)Oc2c(F)cc3[nH]ccc3c2CNC(=O)OC1] |
| 90 | 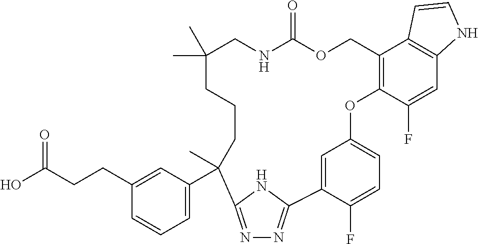 [SMILES: CC1(C)CCCC(C)(c2cccc(CCC(=O)O)c2)c2nnc([nH]2)-c2cc(ccc2F)Oc2c(F)cc3[nH]ccc3c2COC(=O)NC1] |
| 91 | 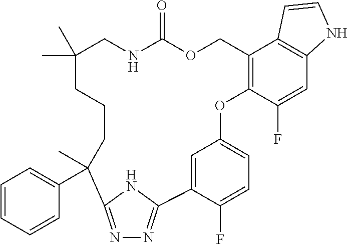 [SMILES: CC1(C)CCCC(C)(c2ccccc2)c2nnc([nH]2)-c2cc(ccc2F)Oc2c(F)cc3[nH]ccc3c2COC(=O)NC1] |
| 92 | 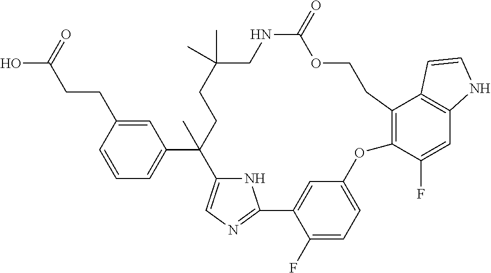 [SMILES: CC1(C)CCC(C)(c2cccc(CCC(=O)O)c2)c2cnc([nH]2)-c2cc(ccc2F)Oc2c(F)cc3[nH]ccc3c2CCOC(=O)NC1] |
| 93A | 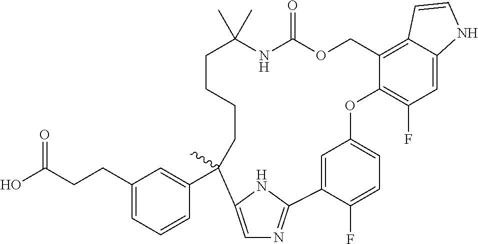 [SMILES: CC1(C)CCCCC(C)(c2cccc(CCC(=O)O)c2)c2cnc([nH]2)-c2cc(ccc2F)Oc2c(F)cc3[nH]ccc3c2COC(=O)N1] |
| 93B | 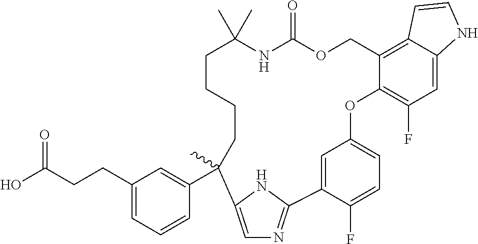 [SMILES: CC1(C)CCCCC(C)(c2cccc(CCC(=O)O)c2)c2cnc([nH]2)-c2cc(ccc2F)Oc2c(F)cc3[nH]ccc3c2COC(=O)N1] |
| 94 | 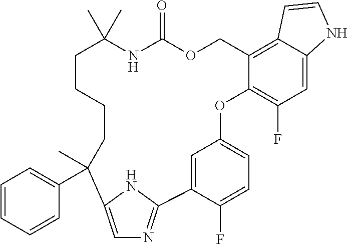 [SMILES: CC1(C)CCCCC(C)(c2ccccc2)c2cnc([nH]2)-c2cc(ccc2F)Oc2c(F)cc3[nH]ccc3c2COC(=O)N1] |
| 95A | 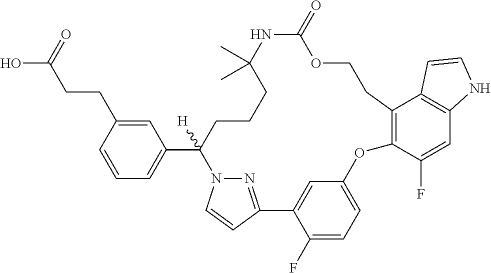 [SMILES: CC1(C)CCCC(c2cccc(CCC(=O)O)c2)n2ccc(n2)-c2cc(ccc2F)Oc2c(F)cc3[nH]ccc3c2CCOC(=O)N1] |
| 95B | 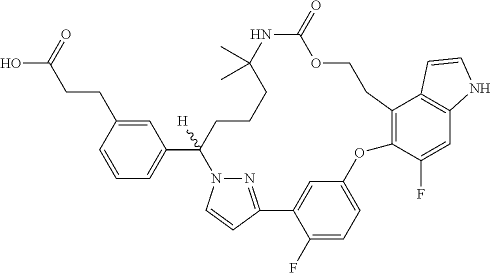 [SMILES: CC1(C)CCCC(c2cccc(CCC(=O)O)c2)n2ccc(n2)-c2cc(ccc2F)Oc2c(F)cc3[nH]ccc3c2CCOC(=O)N1] |
| 96A | 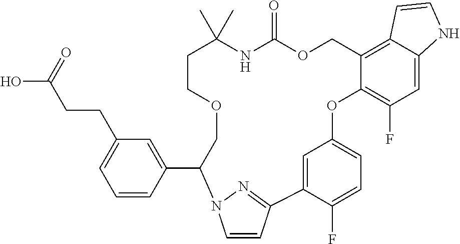 [SMILES: CC1(C)CCOCC(c2cccc(CCC(=O)O)c2)n2ccc(n2)-c2cc(ccc2F)Oc2c(F)cc3[nH]ccc3c2COC(=O)N1] |
| 96B | 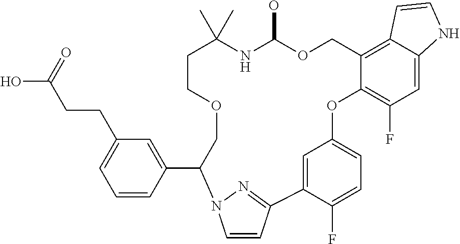 [SMILES: CC1(C)CCOCC(c2cccc(CCC(=O)O)c2)n2ccc(n2)-c2cc(ccc2F)Oc2c(F)cc3[nH]ccc3c2CO[C@H](O)N1] |
| 97A | 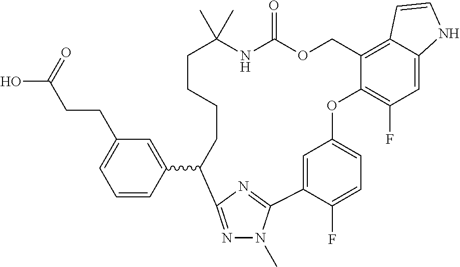 [SMILES: Cn1nc2nc1-c1cc(ccc1F)Oc1c(F)cc3[nH]ccc3c1COC(=O)NC(C)(C)CCCCC2c1cccc(CCC(=O)O)c1] |
| 97B | 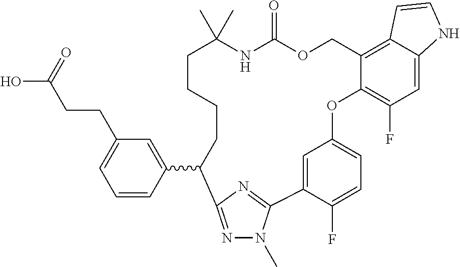 [SMILES: Cn1nc2nc1-c1cc(ccc1F)Oc1c(F)cc3[nH]ccc3c1COC(=O)NC(C)(C)CCCCC2c1cccc(CCC(=O)O)c1] |
| 98A | 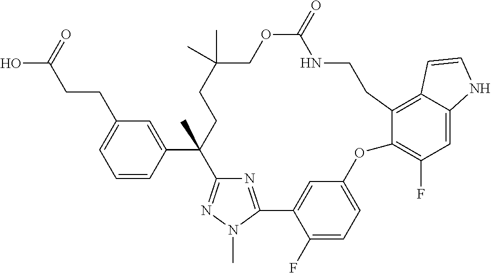 [SMILES: Cn1nc2nc1-c1cc(ccc1F)Oc1c(F)cc3[nH]ccc3c1CCNC(=O)OCC(C)(C)CC[C@@]2(C)c1cccc(CCC(=O)O)c1] |
| 98B | 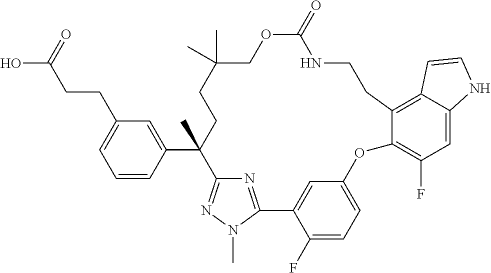 [SMILES: Cn1nc2nc1-c1cc(ccc1F)Oc1c(F)cc3[nH]ccc3c1CCNC(=O)OCC(C)(C)CC[C@@]2(C)c1cccc(CCC(=O)O)c1] |
| 99A | 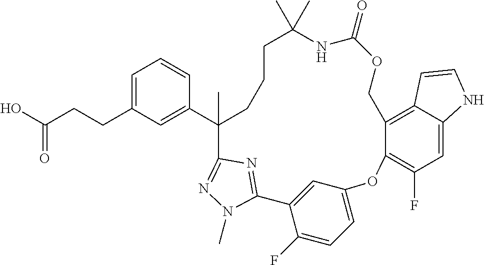 [SMILES: Cn1nc2nc1-c1cc(ccc1F)Oc1c(F)cc3[nH]ccc3c1COC(=O)NC(C)(C)CCCC2(C)c1cccc(CCC(=O)O)c1] |
| 99B | 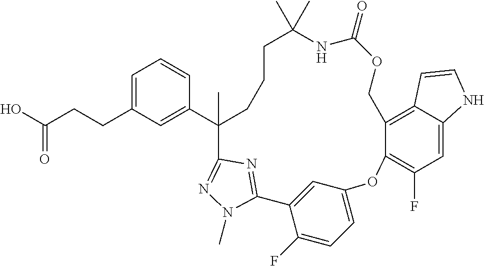 [SMILES: Cn1nc2nc1-c1cc(ccc1F)Oc1c(F)cc3[nH]ccc3c1COC(=O)NC(C)(C)CCCC2(C)c1cccc(CCC(=O)O)c1] |
| 100A | 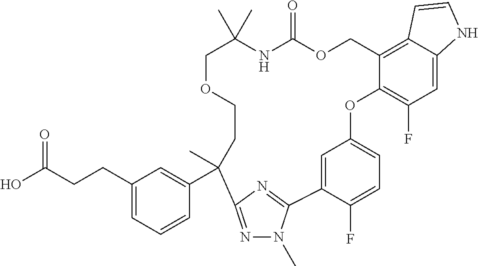 [SMILES: Cn1nc2nc1-c1cc(ccc1F)Oc1c(F)cc3[nH]ccc3c1COC(=O)NC(C)(C)COCCC2(C)c1cccc(CCC(=O)O)c1] |
| 100B | 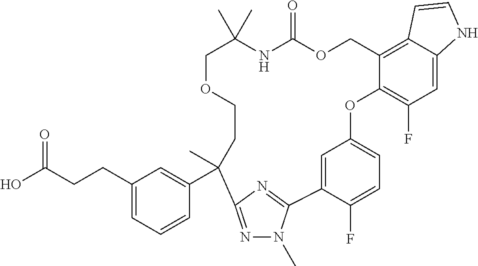 [SMILES: Cn1nc2nc1-c1cc(ccc1F)Oc1c(F)cc3[nH]ccc3c1COC(=O)NC(C)(C)COCCC2(C)c1cccc(CCC(=O)O)c1] |
| 101A | 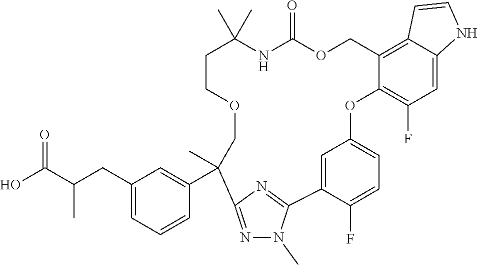 [SMILES: CC(Cc1cccc(C2(C)COCCC(C)(C)NC(=O)OCc3c(c(F)cc4[nH]ccc34)Oc3ccc(F)c(c3)-c3nc2nn3C)c1)C(=O)O] |
| 101B | 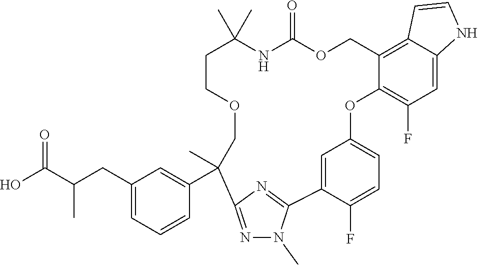 [SMILES: CC(Cc1cccc(C2(C)COCCC(C)(C)NC(=O)OCc3c(c(F)cc4[nH]ccc34)Oc3ccc(F)c(c3)-c3nc2nn3C)c1)C(=O)O] |
| 102 | 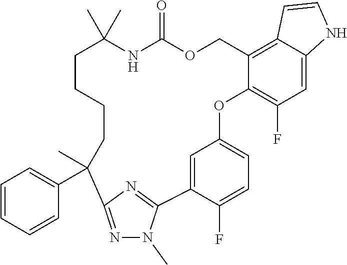 [SMILES: Cn1nc2nc1-c1cc(ccc1F)Oc1c(F)cc3[nH]ccc3c1COC(=O)NC(C)(C)CCCCC2(C)c1ccccc1] |
| 103 | 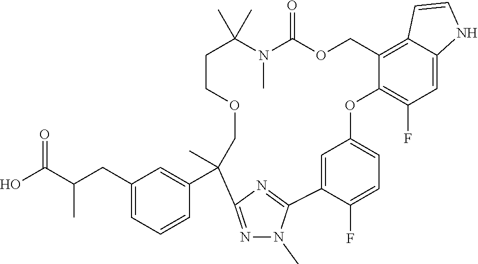 [SMILES: CC(Cc1cccc(C2(C)COCCC(C)(C)N(C)C(=O)OCc3c(c(F)cc4[nH]ccc34)Oc3ccc(F)c(c3)-c3nc2nn3C)c1)C(=O)O] |
| 104 | 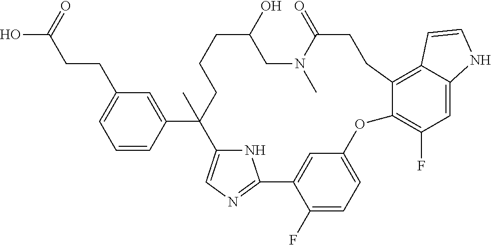 [SMILES: CN1CC(O)CCCC(C)(c2cccc(CCC(=O)O)c2)c2cnc([nH]2)-c2cc(ccc2F)Oc2c(F)cc3[nH]ccc3c2CCC1=O] |
| 105 | 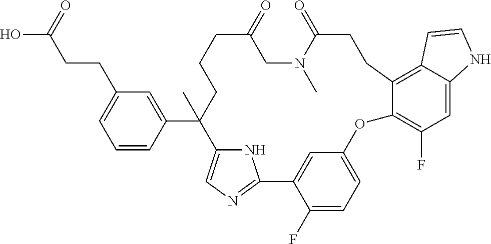 [SMILES: CN1CC(=O)CCCC(C)(c2cccc(CCC(=O)O)c2)c2cnc([nH]2)-c2cc(ccc2F)Oc2c(F)cc3[nH]ccc3c2CCC1=O] |
| 106 | 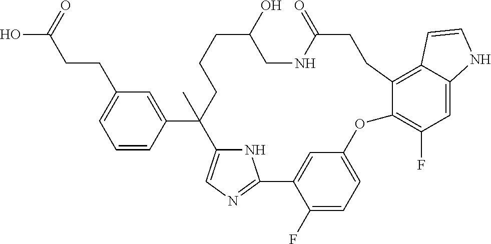 [SMILES: CC1(c2cccc(CCC(=O)O)c2)CCCC(O)CNC(=O)CCc2c(c(F)cc3[nH]ccc23)Oc2ccc(F)c(c2)-c2ncc1[nH]2] |
| 107 | 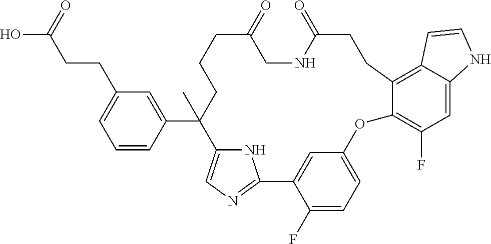 [SMILES: CC1(c2cccc(CCC(=O)O)c2)CCCC(=O)CNC(=O)CCc2c(c(F)cc3[nH]ccc23)Oc2ccc(F)c(c2)-c2ncc1[nH]2] |
| 108 | 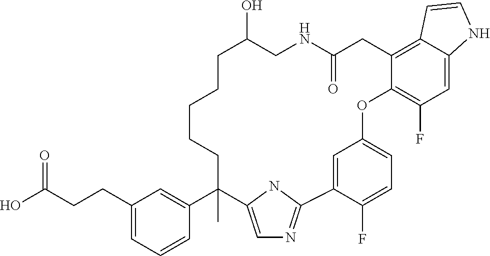 [SMILES: CC1(c2cccc(CCC(=O)O)c2)CCCCCC(O)CNC(=O)Cc2c(c(F)cc3[nH]ccc23)Oc2ccc(F)c(c2)-c2ncc1[nH]2] |
| 109 | 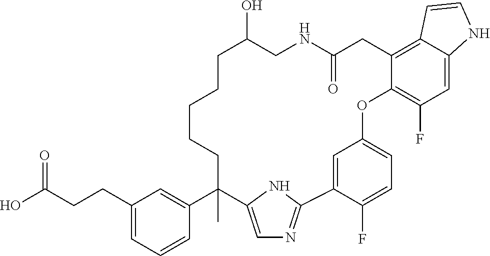 [SMILES: CC1(c2cccc(CCC(=O)O)c2)CCCCCC(O)CNC(=O)Cc2c(c(F)cc3[nH]ccc23)Oc2ccc(F)c(c2)-c2ncc1[nH]2] |
| 110 | 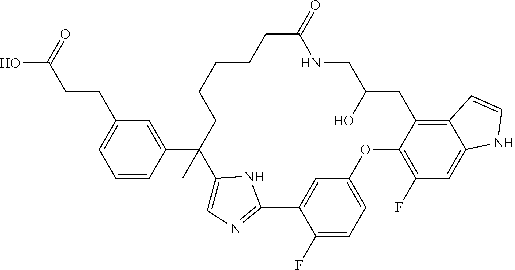 [SMILES: CC1(c2cccc(CCC(=O)O)c2)CCCCCC(=O)NCC(O)Cc2c(c(F)cc3[nH]ccc23)Oc2ccc(F)c(c2)-c2ncc1[nH]2] |
| 111 | 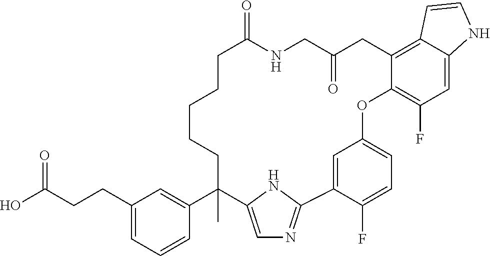 [SMILES: CC1(c2cccc(CCC(=O)O)c2)CCCCCC(=O)NCC(=O)Cc2c(c(F)cc3[nH]ccc23)Oc2ccc(F)c(c2)-c2ncc1[nH]2] |
| 112 | 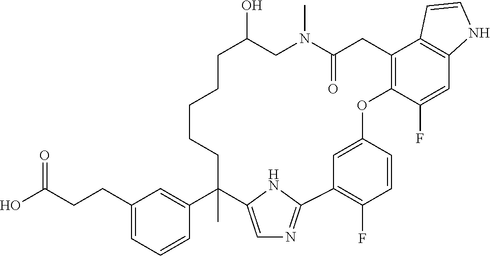 [SMILES: CN1CC(O)CCCCCC(C)(c2cccc(CCC(=O)O)c2)c2cnc([nH]2)-c2cc(ccc2F)Oc2c(F)cc3[nH]ccc3c2CC1=O] |
| 113 | 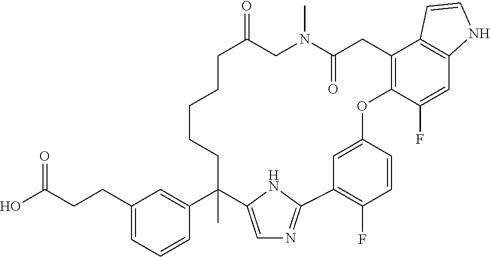 [SMILES: CN1CC(=O)CCCCCC(C)(c2cccc(CCC(=O)O)c2)c2cnc([nH]2)-c2cc(ccc2F)Oc2c(F)cc3[nH]ccc3c2CC1=O] |
| 114 | 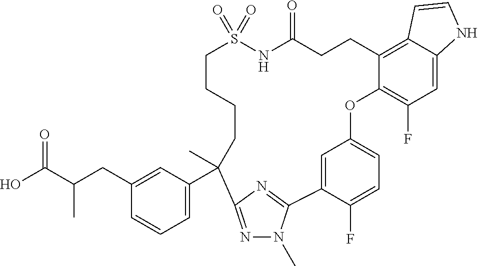 [SMILES: CC(Cc1cccc(C2(C)CCCCS(=O)(=O)NC(=O)CCc3c(c(F)cc4[nH]ccc34)Oc3ccc(F)c(c3)-c3nc2nn3C)c1)C(=O)O] |
| 115 | 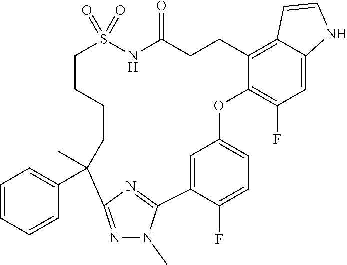 [SMILES: Cn1nc2nc1-c1cc(ccc1F)Oc1c(F)cc3[nH]ccc3c1CCC(=O)NS(=O)(=O)CCCCC2(C)c1ccccc1] |
| 116 | 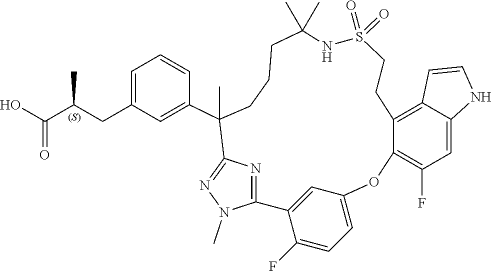 |
| 117A | 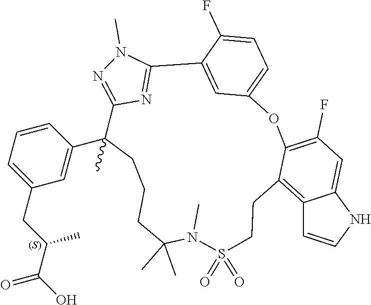 |
| 117B | 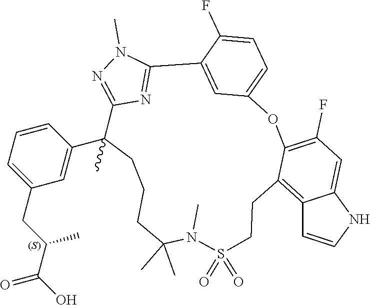 |
| 118 | 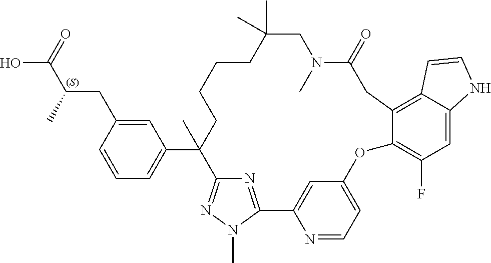 |
| 119 | 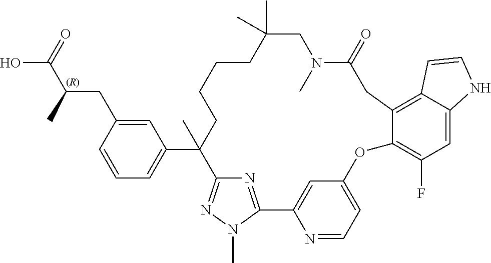 |
| 120 | 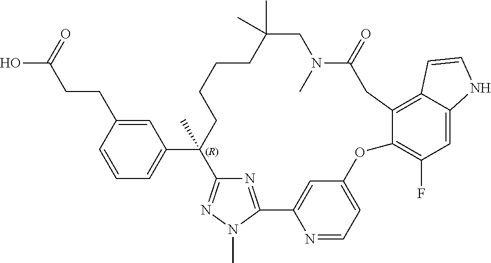 |
| 121 | 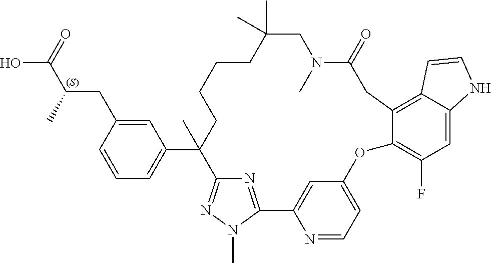 |
| 122 | 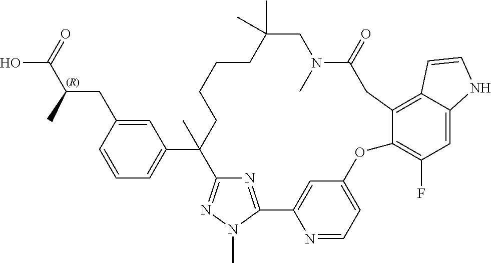 |
| 123 | 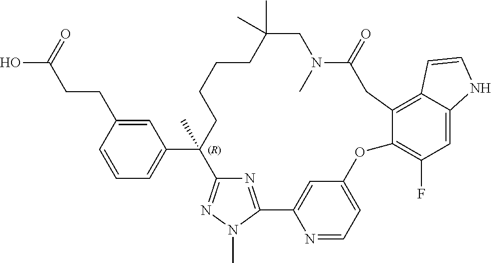 |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “haloalkyl” refers to a straight or branched alkyl group that is substituted with one or more halogen atoms.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group. The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; CH═CHPh, which may be substituted with R^{∘}; (CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, (CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; N(OR^{∘})R^{∘}; C(NH)NR^{∘}₂; P(O)₂R^{∘}; P(O)R^{∘}₂; OP(O)R^{∘}₂; OP(O)(OR^{∘})₂; SiR^{∘}₃; (C₁₋₄ straight or branched alkylene)O—N(R^{∘})₂; or (C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, (CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, OSiR^{•}₃, —C(O)SR^{•}, —(C₁₋₄ straight or branched alkylene)C(O)OR^{•}, or SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CFTR-associated disease or disorder.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Defects in the function of the CFTR ion channel result from loss of function mutations of CFTR. Such mutations lead to exocrine gland dysfunction, abnormal mucociliary clearance, and cause cystic fibrosis. The most common CFTR mutation in Cystic Fibrosis (CF) patients leads to the specific deletion of three nucleotides of the codon for phenylalanine at position 508. This mutation, which is found in ˜70% of CF patients worldwide, is referred to as “ΔF508”. The ΔF508 mutation decreases the stability of the CFTR NBD1 domain and limits CFTR interdomain assembly. Since CF is an autosomal recessive disease, a CF patient harboring the ΔF508 CFTR mutation must also carry a second defective copy of CFTR. Approximately 2000 different CF-causing CFTR mutations have been identified in CF patients. CF patients harboring the ΔF508 CFTR mutation can be homozygous for that mutation (ΔF508/ΔF508). CF patients can also be ΔF508 heterozygous, if the second CFTR allele such patients carry instead contains a different CFTR loss of function mutation. Such CFTR mutations include, but are not limited to, G542X, G551D, N1303K, W1282X, R553X, R117H, R1162X, R347P, G85E, R560T, A455E, A1507, G178R, S549N, S549R, G551S, G970R, G1244E, S1251N, 51255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator.

As used herein, the term “CFTR corrector” refers to a compound that increases the amount of functional CFTR protein to the cell surface and thus enhances CFTR channel function. The CFTR correctors partially “rescue” misfolding of CFTR, thereby enabling the maturation and functional expression of CFTR protein harboring a CF causing mutation on the cell surface. Examples of correctors include, but are not limited to, VX-809, VX-661, VX-152, VX-440, VX-983, and GLPG2222. Such compounds may interact directly with CFTR protein, modifying its folding and conformational maturation during synthesis.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genistein and GLPG1837.

As used herein, the term “CFTR pharmacological chaperone” (PC) refers to compounds that stabilize the CFTR protein in its native state by binding directly to the protein.

As used herein, the term “CFTR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A-beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (A) and a pharmaceutically acceptable carrier, adjuvant, or vehicle. In some embodiments, the amount of compound in compositions contemplated herein is such that is effective to measurably modulate CFTR, or a mutant thereof, in a biological sample or in a patient. In certain embodiments, the amount of compound in compositions of this disclosure is such that is effective to measurably modulate CFTR, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, the amount of compound in compositions contemplated herein is such that is effective to measurably modulate a protein, particularly at CFTR, or a mutant thereof, in a biological sample or in a patient. In certain embodiments, the amount of compound in compositions of this disclosure is such that is effective to measurably modulate CFTR, or a mutant thereof, in a biological sample or in a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (A) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (A) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (A) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (A) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (A) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

As discussed above, CFTR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CFTR reaches the plasma membrane, indicating that CFTR has energetic instability of individual NBDs, a slow domain assembly, and relatively fast ERAD kinetics which all contribute to inefficient folding and sensitize CFTR to structural perturbations by mutations.

In wild-type CFTR, the NBD1 domain folds co-translationally while other domains fold post-translationally. Mutated ΔF508 CFTR has impaired NBD1 folding but its backbone structure and thermodynamic stability are similar to wild-type CFTR. With delayed folding kinetics, mutated ΔF508 CFTR NBD1 has an increased folding activation energy. Lack of proper folding results in hydrophobic residues being exposed to the surface of NBD1 which causes aggregation with other CFTR proteins. Thus, the aggregation temperature of mutated CFTR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result in the CFTR biosynthetic processing, underscoring the need for interface stability as well.

The disclosed CFTR correctors can interact with the NBD domain to stabilize the correct folded position R, such that CFTR is not labeled for elimination from the cell. The preservation of correct folding enables CFTR to function as a chloride ion channel at wild-type levels. In some embodiments, disclosed CFTR correctors can enhance the performance of wild-type CFTR.

CFTR stabilizers can function in combination with other therapeutic agents such as CFTR correctors that promote Δ508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of Formula (A), or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of Formula (A) or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol, s-nitroglutathione) reductase inhibitors, and a CRISPR Cas correction therapy or system (as described in US 2007/0022507 and the like). In some embodiments, combination therapy includes administration of a compound described herein with a compound that modulates CFTR protein or ABC protein activities (e.g., as described in WO2018167690A1 and the like)

In certain embodiments, the method of treating a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

In certain embodiments, the method of preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1, 3]dioxol-5-yl)cyclopropanecarboxamido)-3-methylpyridin-2-yl) benzoic acid, VX-661 (Tezacaftor, I-(2,2-difluoro-1,3-benzodioxol-5-yl)-N-[I-[(2R)-2,3-dihydroxypropyl]-6-fluoro-2-(2-hydroxy-1, I-dimethylethyl)-IH-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1, 2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. 62/944,141, 62/944,158 and 62/944,188, each of which is incorporated by reference in its entirety.

Non-limiting examples of anti-inflammatory agents are N6022 (3-(5-(4-(IH-imidazol-I-yl)10 phenyl)-I-(4-carbamoyl-2-methylphenyl)-′H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodilator, an anti-infective (such as tazobactam, piperacillin, rifampin, meropenum, ceftazidime, aztreonam, tobramycin, fosfomycin, azithromycin, amitriptyline, vancomycin, gallium and colistin), an anti-infective agent, an anti-inflammatory agent, a CFTR modulator other than a compound of the present disclosure, and a nutritional agent. Additional therapeutic agents can include treatments for comorbid conditions of cystic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG2451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanec arboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of Formula (A), with a second therapeutic agent may have a synergistic effect in the treatment of cancer and other diseases or disorders mediated by adenosine. In other embodiments, the combination may have an additive effect.

### Exemplification

### Abbreviations:

Boc: tert-butyloxycarbonyl
DEA: diethyl amine
DBU: 1,8-Diazabicyclo[5.4.0]undec-7-ene
DMSO: dimethyl sulfoxide
dppf: 1,1′-Bis(diphenylphosphino)ferrocene
DTT: dithiothreitol
ESI: electron spray ionization
HATU: 1-[Bis(dimethylamino)methylene]-1H-1,2,3-triazolo[4,5-b]pyridinium 3-oxide hexafluorophosphate
HPLC: high performance liquid chromatography
LC-MS: liquid chromatography-mass spectrometry
NIS: N-iodosuccinimide
Pd/C: Palladium on carbon
SFC: supercritical fluid chromatography
TBS: tert-Butyldimethylsilyl
TIPS: Triisopropylsilyl
THF: tetrahydrofuran
THP: tetrahydropyran
Ts: tosyl

### General Formula:

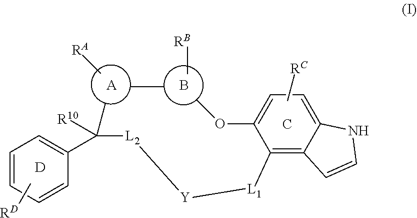

### General Procedures

The compounds of the present disclosure can be better understood in connection with the following synthetic schemes and methods which illustrate means by which the compounds of the Formula (I) can be prepared. The compounds of this disclosure can be prepared by a variety of synthetic procedures illustrated in Schemes I to IX.

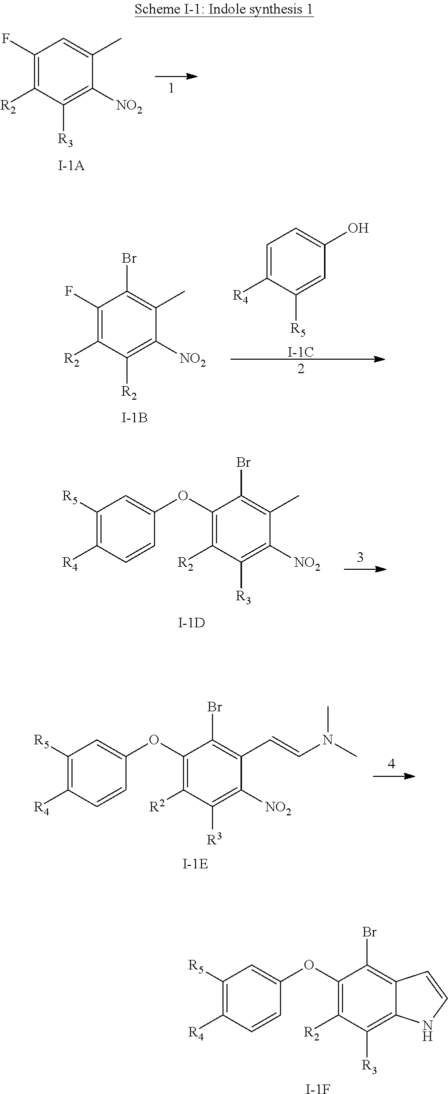

The intermediate I-1F may be prepared as illustrated in Scheme I-1. Properly substituted methyl nitrobenzene (I-1A) is brominated (step 1) to give bromide I-1B. Compound I-1B is condensed with phenol I-1C (step 2) to give the ether I-1D. Treatment of I-1D with N,N-dimethylformamide dimethyl acetal (step 3) is used to form 1-1E. Reductive-cyclization (step 4) of 1-1E results in the intermediate I-1F.

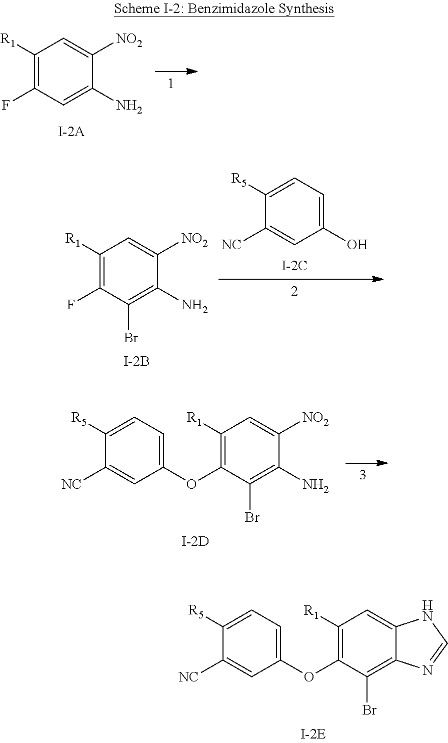

Nitroaniline I-2A was brominated to I-2B, which was then substituted with I-2C, then reduced and cyclized with formic acid under proper conditions to afford benzimidazole intermediate I-2E

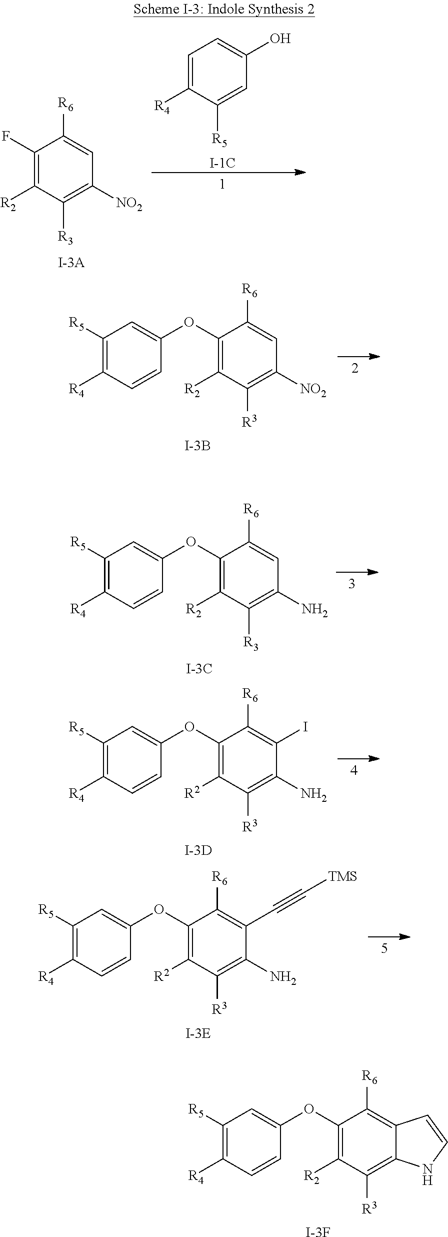

Scheme I-3 describes an alternative method to prepare intermediate I-1F. Intermediate I-3A is condensed with phenol I-1C (step 1) to yield intermediate I-3B. Reduction of the nitro group to amine (step 2) affords I-3C, to which iodine is introduced (step 3) to give intermediate I-3D. This intermediate is then coupled with protected acetylene to yield intermediate I-3E (step 4). Cyclization of I-3E (step 5) yields the key intermediate I-3F.

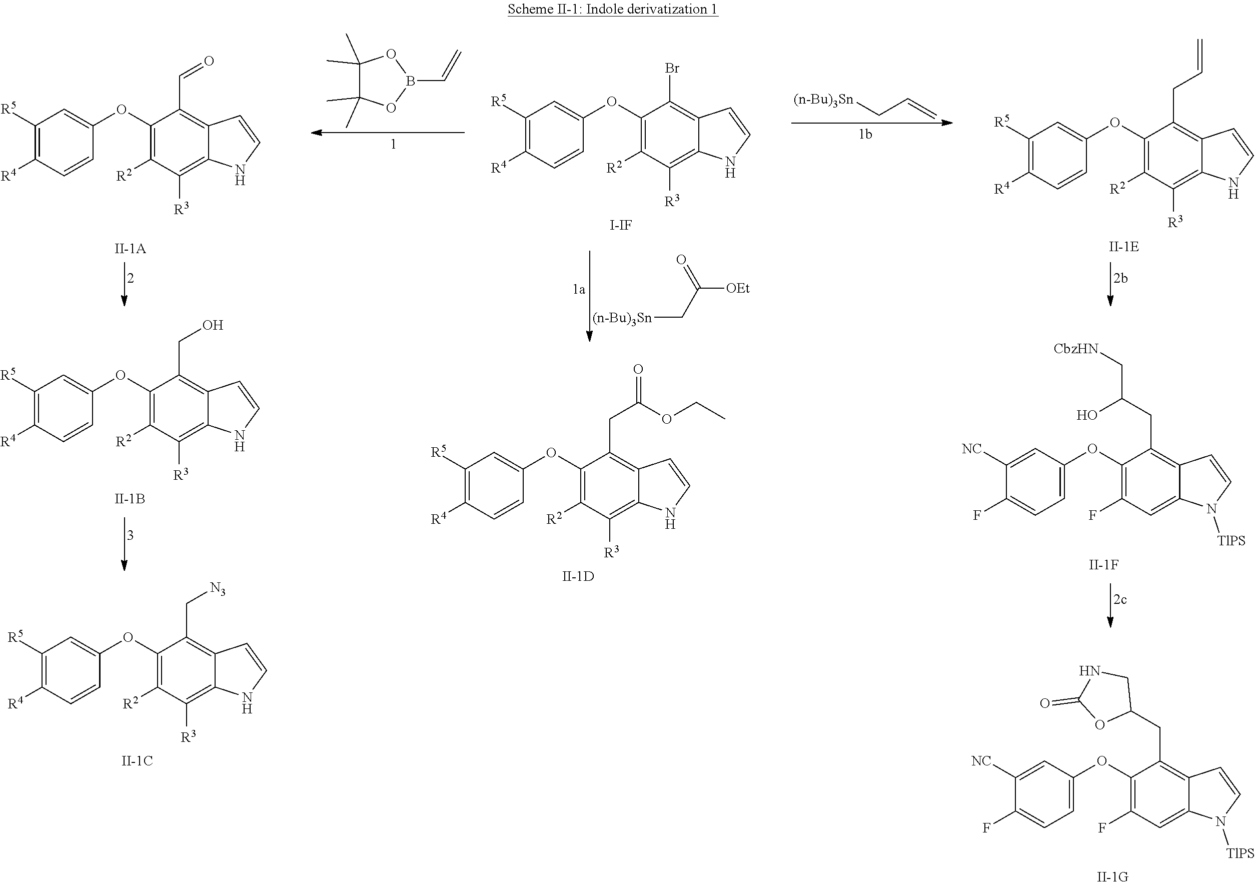

The indole I-1F can be further derivatized as shown in Scheme II-1. The intermediate I-1F is coupled with vinyl boron ester to derive an alkene which undergoes oxidative cleavage to yield aldehyde II-1A (step 1). Reduction of the aldehyde gives an alcohol II-1B (Step 2), which is converted to azide II-1C, through methods such as mesylate formation/sodium azide substitution.

Certain side chains at C₄ of the indole can also be installed via a Stille coupling. For example, in Scheme II-1, bromide I-1F is coupled (Step 1a) with Stille reagent to obtain an ester II-1D. Further, the intermediate I-1F can also be coupled with organic tin reagent. For example, I-1F is coupled with allyl(tributyl)stannane catalyzed by lithium chloride and bis-(triphenylphosphine)palladium(II) chloride (Step 1b) to give allyl indole (II-1E), which is further derivatized (step 2b) into a proper coupling partner, such as II-1F and II-1G.

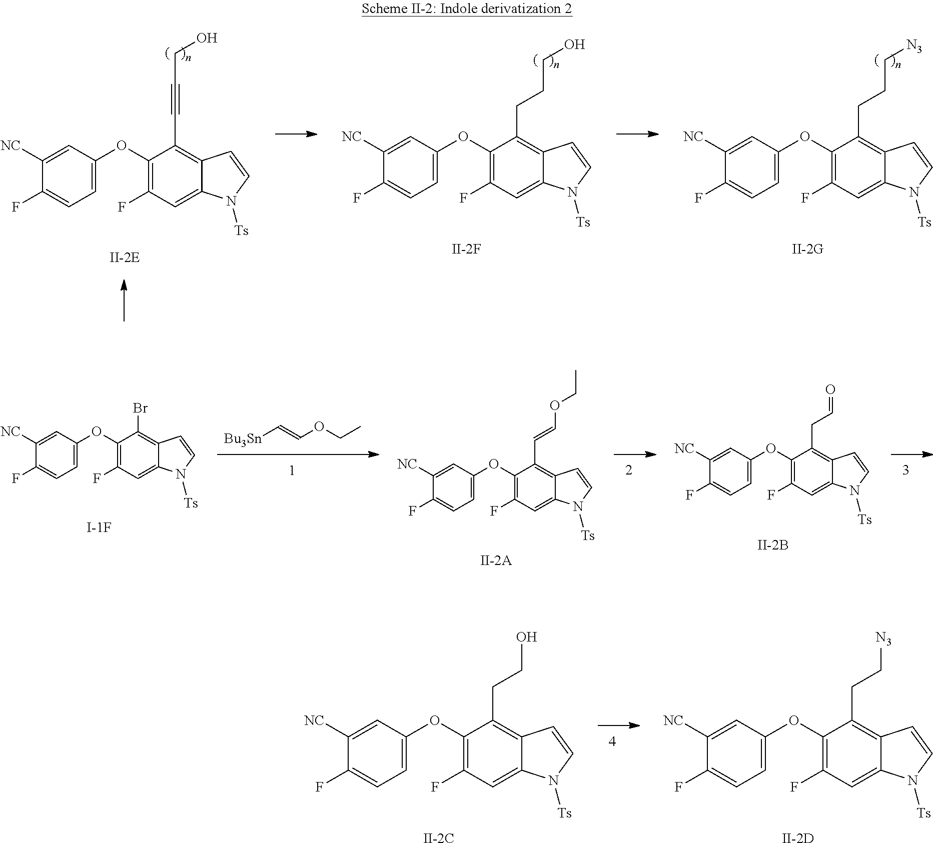

Following this direction further, I-1F is coupled with organo-tin agent (Step 1) to give alkyl derivative II-2A which is converted into an aldehyde II-2B (Step 2). After reduction (Step 3) and activation using the proper agent, such as tosylate or mesylate, the alcohol is converted into azide (II-2D) (Step 4) which can be used for the coupling reaction (see later). I-1F is also subjected to Sonogashira coupling to afford II-2E, which undergoes hydrogenation to give alcohol II-2F. This alcohol is further converted to azide 11-2G.

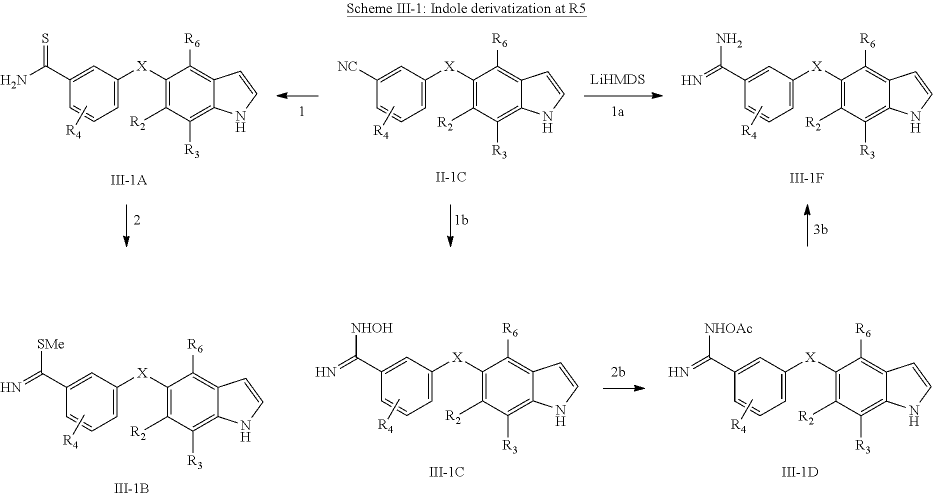

Methyl benzimidothioate (III-1B) may be derived from a 2-step sequence as shown in Scheme III-1. Nitrile derivative (II-1C) is converted to thioamide (III-1A) (step 1). The resultant thioamide is treated with an active methyl source such as iodomethane to obtain methyl benzimidothioate (III-1B) (step 2). Similarly, the intermediate amidine (III-1F) can be prepared from the corresponding nitrile I-1C in one step when treated with lithium bis(trimethylsilyl)amide (step 1a). Alternatively, amidine III-1F may be prepared from a three-step sequence. Addition of hydroxylamine to nitrile II-1C results in hydoxyamidine (III-1C) (step 1b), acetylation (step 2b), followed by hydrogenation (Step 3b) to afford amidine III-1F.

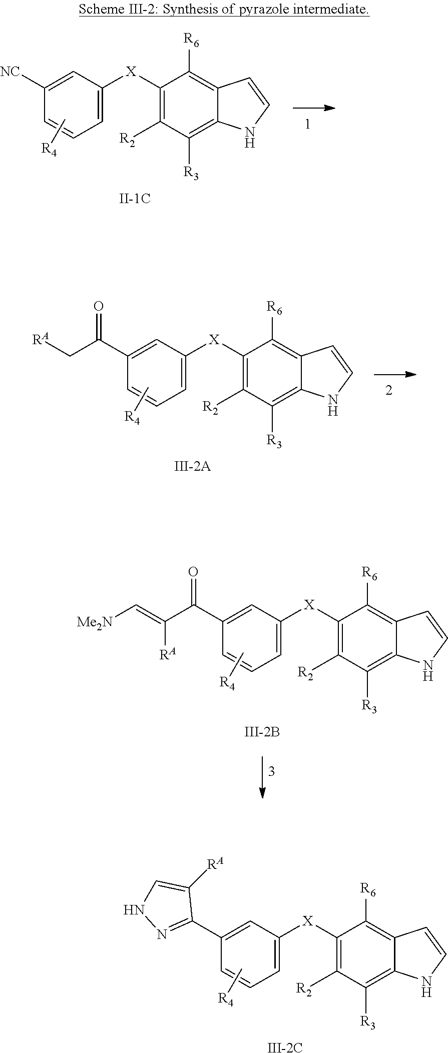

Intermediate III-2C may be synthesized through a three-step sequence (Scheme III-2). The nitrile I-1C or other related precursors is converted into the ketone III-2A (Step 1). This ketone is condensed with dimethylformamide dimethyl acetal to yield intermediate III-2B (Step 2). The resulting enamine is cyclized with hydrazine to form pyrazole III-2C (step 3).

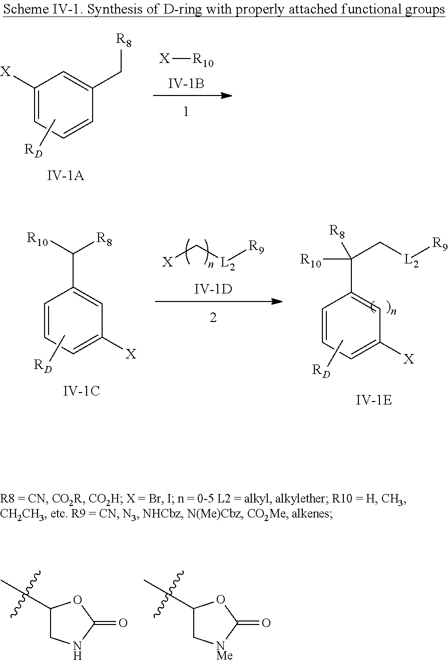

Scheme IV-1 describes the synthesis of the key intermediate IV-E from readily available starting material IV-1A. In Step 1, alkylation is facilitated by a strong base, such as LDA, to form Intermediate IV-C. The process is repeated with another alkylating agent IV-D (Step 2) to yield intermediate IV-E.

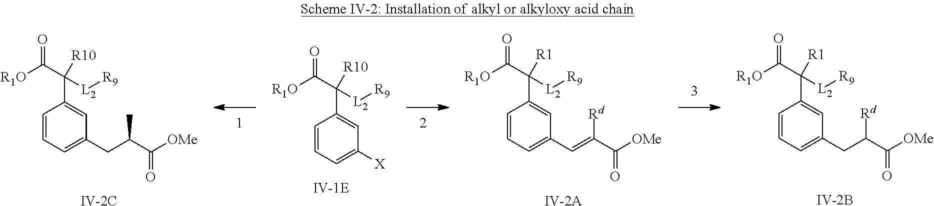

The installation of alkyl acid chains is illustrated in Scheme IV-2. Negishi coupling or other related coupling reactions were used to form IV-2C (step 1). Heck reaction (step 6) followed by hydrogenation (step 7) affords IV-2B.

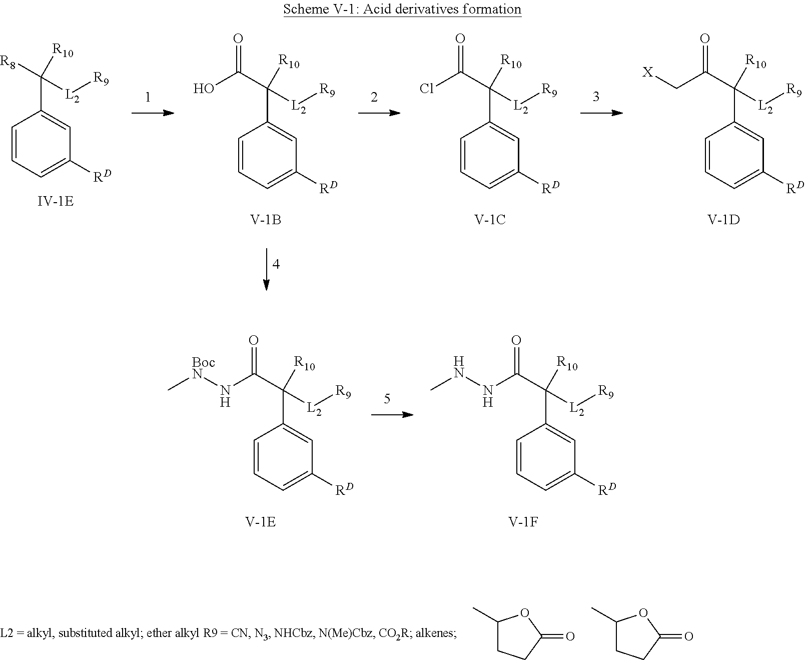

Acid V-1B was obtained from corresponding ester/nitrile IV-1E either through acid treatment (when R8 is tert-butyl ester), saponification (when R8 is nitrile, or esters like methyl or ethyl ester), or hydrogenation (when R8 is benzyl ester). Bromo or chloro ketone V-1D is prepared from corresponding acid (V-1B), which is converted to acetyl chloride (step 2). The acetyl chloride V-1C was treated with diazomethane, then decomposed with either hydrogen chloride in 1,4-dioxane or hydrobromic acid in acetic acid to afford bromo/chloro ketone V-1D (step 3). The acid V-1B can also be directly converted to (monosubstituted) hydrazides by coupling with protected (monosubstituted) hydrazine (Step 4) followed by deprotection (step 5).

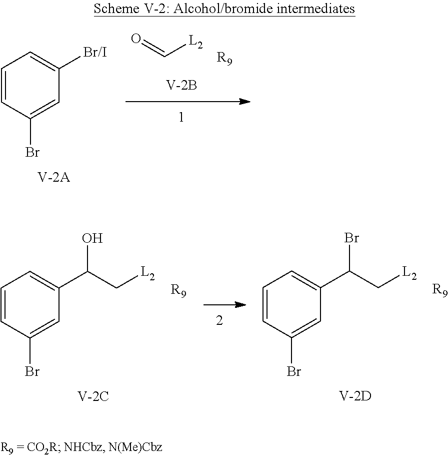

Intermediates V-2D are used for the corresponding pyrazole macrocyclic compounds. Scheme V-2 illustrated the preparation method for V-2D. Mono metal-halogen exchange of V-2A followed by treatment with aldehyde V-2B resulted in alcohol V-2C, which can be converted to bromide V-2D.

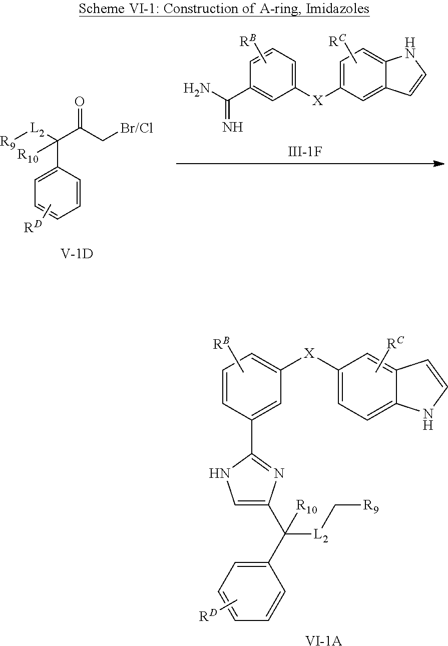

The imidazole A ring intermediate VI-1A is prepared through the reaction of a bromo/chloro-ketone V-1D with amidine III-1F in the presence of a proper base and a suitable solvent in a one-step synthesis.

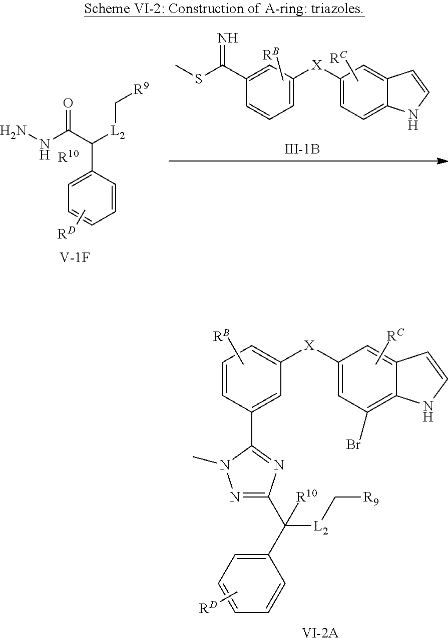

The triazole A ring intermediate VI-2A is formed through the reaction of hydrazide V-1F with methyl benzimidothioate III-1B in a proper solvent system, like pyridine.

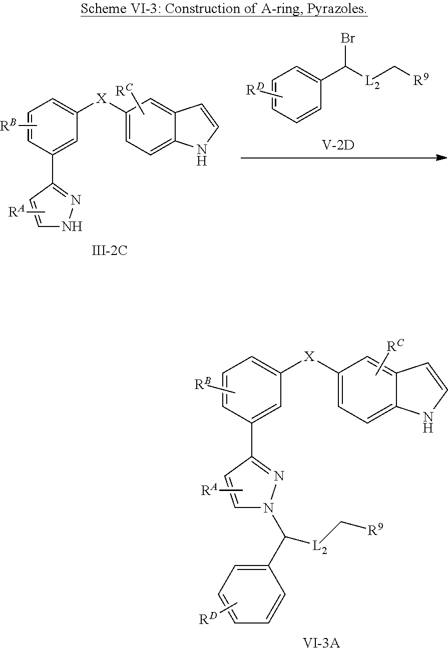

The pyrazole A-ring intermediate with the proper attached functional groups VI-3A is synthesized via alkylation of intermediate III-2C with the proper alkylating agent V-2D at ambient or elevated temperature in the presence of a base.

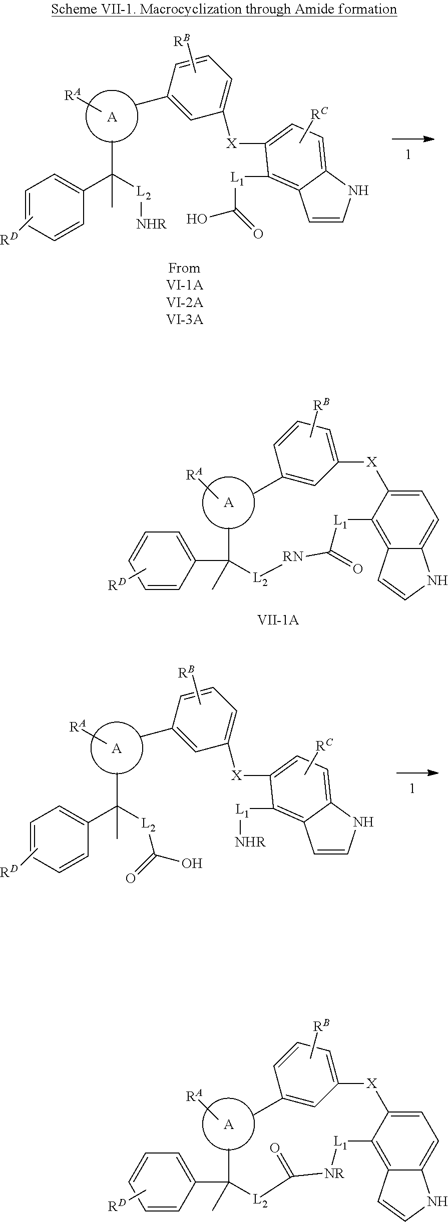

Macrocyclization may be achieved by the amide bond formation in the presence of a dehydration agent like EDCI, HATU, in the present of bases like diisopropylethylamine, and HOBt, from acid/amine precursor obtained from VI-1A, 2A, 3A via a proper functional group manipulation.

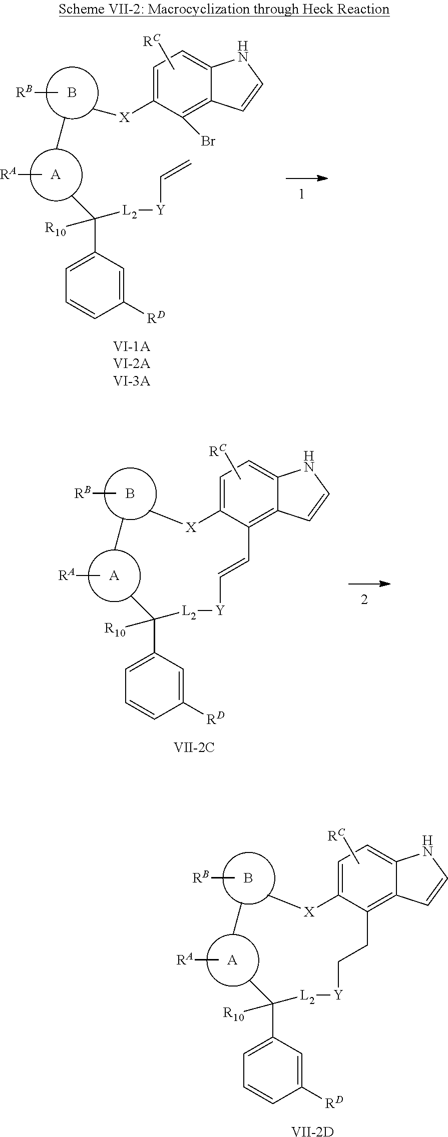

Macrocyclization can also be achieved through a Heck reaction as shown in Scheme VII-2. Vinyl bromide, from VI-1A, VI-2A and VI-3A, with a proper catalyst system, undergoes macrocyclization to afford macrocyclic intermediate VII-2C, which is hydrogenated to give macrocycle VII-2D.

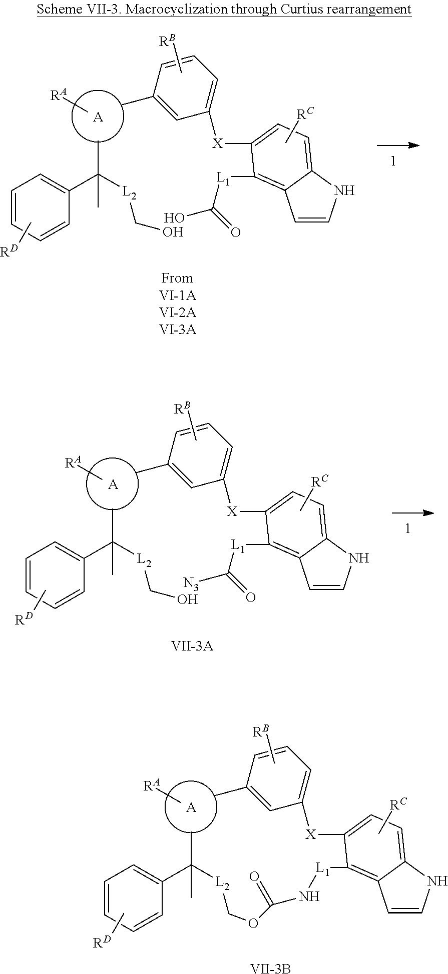

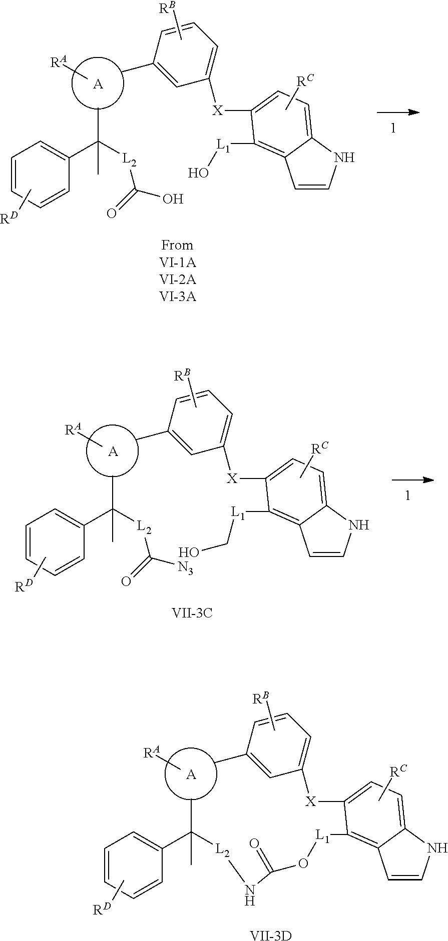

Acid alcohol obtained after functional group manipulation of VI-1A, VI-2A, VI-3A, was converted to carbonyl azide VII-3A and VII-3C, then cyclized to carbamate VII-3B and VII-3D at elevated temperature, in the presence of a base such as triethylamine.

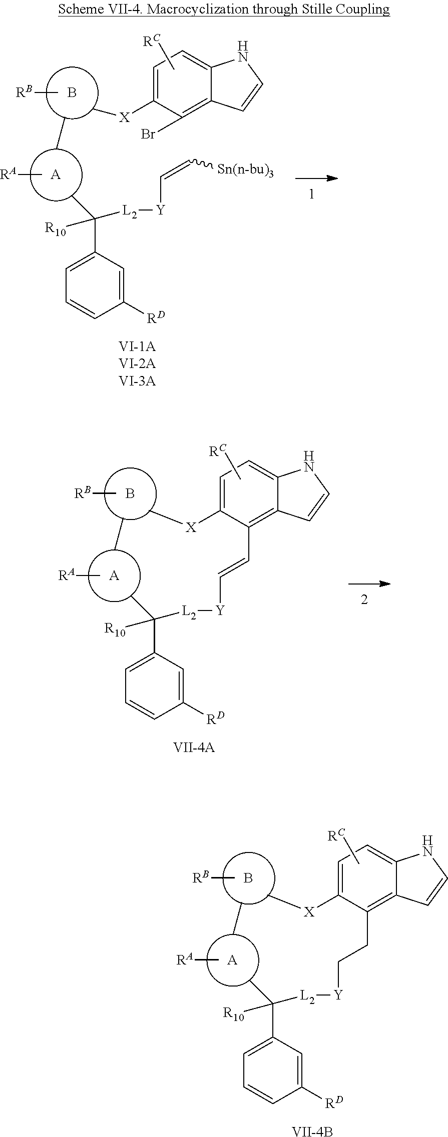

Stille Coupling is also successfully applied in the macrocyclization reaction as illustrated in Scheme VII-4. In step 1, vinyltributylstannane functional group in VI-1A, VI-2A, VI-3A is coupled with halo functional group in ring C to yield Intermediate VII-4A. After hydrogenation the final compound or intermediate VII-4B is obtained.

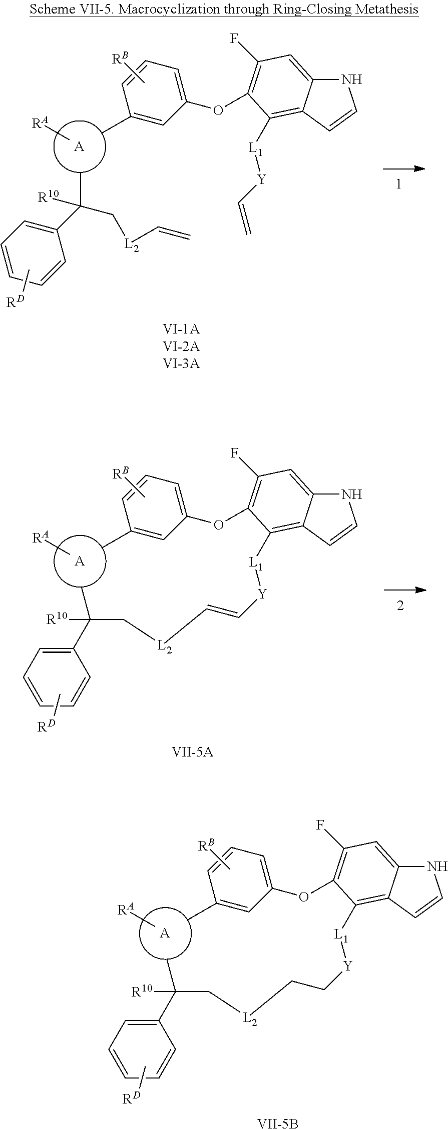

As illustrated Scheme VII-5, the proper starting material with required alkenes is subjected to Hoveyda-Grubbs catalytic conditions (Step 1) to form an alkene. After hydrogenation, the macrocycle (VII-5B) is obtained.

### Analytical Methods:

Analytical Procedures ¹H NMR spectra were recorded with Bruker AC 400 MHz apparatus. Chemical shift (δ) is quoted in parts per million (ppm) and coupling constants (J) in hertz (Hz).

The following liquid chromatography-mass spectrometry (LC-MS) methods were used.

### LC-MS Method 1:

Spectra were obtained with UPLC Acquity device of Waters for liquid chromatography part, coupling with mass spectrometer ZMD of Waters. This system was piloted by MassLynx v4.1 software. Detection was made in UV at 220 nm. Operational conditions for liquid chromatography part are the following: Column: Assentis Express C18 50×2.1 mm, 2.7μ Supelco Eluent: Way A: Water+0.02% trifluoroacetic acid; Way B: acetonitrile+0.014% trifluoroacetic acid; Gradient: T=0 minutes: 2% B; T=1 minutes: 98% B; T=1.3 minutes: 98% B, T=1.33 minutes: 2% B, T=1.5 minutes following injection; Flow: 1 mL/minutes; Temperature: 55° C. SQD: ESI+ 30V UV: 220 nm Injection: 0. 2 μL.

### LC-MS Method 2:

Mobile phase: A: water (10 mM ammonium bicarbonate), B: acetonitrile; Gradient: 5% B increase to 95% B within 1.5 minutes, 95% B for 1.5 minutes, back to 5% B within 0.01 minutes. Flow Rate: 1.8 mL/minute; Column: XBridge, 3.5 μm, 50×4.6 mm; Oven Temperature: 50° C.

### LC-MS Method 3:

Mobile Phase: A: water (0.01% trifluoroacetic acid), B: Acetonitrile (0.01% trifluoroacetic acid); Gradient: 5% B increased to 95% B within 1.3 minutes, 95% B for 1.5 minutes, back to 5% within 0.01 minutes; Flow Rate: 2 mL/minute; Column: Sunfire, 50×4.6 mm, 3.5 μm; Column Temperature: 50° C.

### LC-MS Method 4:

LC-Mass Method: Mobile Phase: A: water (0.1% formic acid), B: acetonitrile (0.1% formic acid); Gradient: 5% B increase to 95% B within 1.3 minutes, 95% B for 1.5 minutes, back to 5% B within 0.01 min. Flow Rate: 2 mL/minute; Column: Sunfire C18, 4.6×50 mm, 3.5 μm.

### LC-MS Method 5:

Mobile Phase: A: water (0.01% trifluoroacetic acid), B: acetonitrile (0.01% trifluoroacetic acid); Gradient: 5% B for 0.2 minutes, increase to 95% B within 1.5 minutes, 95% B for 1.5 minutes, back to 5% B within 0.01 minutes; Flow Rate: 2 mL/minute; Column: Sunfire, 50×4.6 mm, 3.5 μm; Column Temperature: 50° C.

### LC-MS Method 6:

Mobile Phase: A: water (0.05% trifluoroacetic acid), B: acetonitrile (0.05% trifluoroacetic acid); Elution program: Gradient from 5% to 100% B in 1.3 minutes at 2 mL/minute; Temperature: 50° C.; Column: SunFire C18, 50×4.6 mm, 3.5 μm.

### LC-MS Method 7:

Mobile Phase: A: water (0.01% trifluoroacetic acid), B: acetonitrile (0.01% trifluoroacetic acid); Gradient: 5% B for 0.2 minutes, increase to 95% B within 1.3 minutes, 95% B for 1.5 minutes, back to 5% B within 0.01 minutes; Flow Rate: 2 mL/minute; Column: Sunfire, 50×4.6 mm, 3.5 μm; Column Temperature: 50° C.

### LC-MS Method 8:

Mobile Phase: A: water (0.01% trifluoroacetic acid), B: acetonitrile (0.01% trifluoroacetic acid); Gradient: 10% to 95% in 1 minute. Flow Rate: 2 mL/minute; Column: Sunfire, 3.5 μm, 50×4.6 mm; Oven Temperature: 50° C.

### LC-MS Method 9:

Mobile Phase: A: water (0.01% trifluoroacetic acid), B: acetonitrile (0.01% trifluoroacetic acid) Gradient: 5% B for 0.2 minutes, increase to 95% B within 1.3 minutes, 95% B for 1.5 minutes, back to 5% B within 0.01 minutes; Flow Rate: 1.8 mL/minute; Column: Sunfire, 50×4.6 mm, 3.5 μm; Column Temperature: 50° C.

### LC-MS Method 10:

Mobile Phase: Column: SunFire C18, 4.6×50 mm, 3.5 μm; Mobile phase: A: water (0.05% trifluoroacetic acid), B: acetonitrile (0.05% trifluoroacetic acid); Elution program: Gradient from 5 to 100% of B in 2.5 minutes at 2 mL/minutes; Temperature: 50° C.

### LC-MS Method 11:

Mobile Phase: A: water (10 mM ammonium carbonate), B: acetonitrile; Gradient: 5% B increase to 95% B within 1.2 minutes, 95% B for 1.5 minutes, back to 5% B within 0.01 minutes. Flow Rate: 1.8 mL/minute; Column: XBridge, 3.5 μm, 50×4.6 mm; Oven Temperature: 50° C.

### LC-MS Method 12:

Mobile phase: A: water (0.1% formic acid), B: acetonitrile (0.1% formic acid); Gradient: B=5%-95% in 1.3 minutes; Flow rate: 1.8 mL/minute; Column: Xbridge, 50×4.6 mm, 3.5 μm.

### LC-MS Method 13:

Mobile phase: A: water (0.01% trifluoroacetic acid), B: acetonitrile (0.01% trifluoroacetic acid); Elution program: Gradient from 5 to 95% of B in 2.5 minutes at 2 mL/minute; Column Temperature: 50° C.

### LC-MS Method 14:

Mobile phase: A: 2.5 mM trifluoroacetic acid in water, B: 2.5 mM trifluoroacetic acid in acetonitrile; Gradient: B=10%-95% in 1.0 minute; Flow rate: 1.5 mL/minute; Column: Xbridge-Cis, 30×4.6 mm, 2.5 μm.

### LC-MS Method 15:

Mobile Phase: A: water (0.01% trifluoroacetic acid), B: acetonitrile (0.01% trifluoroacetic acid); Gradient: 5% B for 0.2 minutes, increase to 95% B within 1.5 minutes, 95% B for 3 minutes, back to 5% within 0.01 minutes; Flow Rate: 2 mL/minute; Column: Sunfire, 50×4.6 mm, 3.5 μm; Column Temperature: 50° C.

### LC-MS Method 16:

Mobile phase: A: water (10 mM ammonium bicarbonate), B: acetonitrile; Gradient: B=5% B increase to 95% B within 1.4 minutes; 95% B for 1.6 minutes, back to 5% B within 0.01 minutes; Flow rate: 1.8 mL/minute; Column: Xbridge-C18, 50×4.6 mm, 3.5 μm; Column Temperature: 50° C.

### LC-MS Method 17:

Column: XBridge C18, 4.6×50 mm, 3.5 μm; Mobile phase: A: water (10 mM ammonium bicarbonate), B: acetonitrile; Elution program: Gradient from 10 to 95% of B in 1.5 minutes at 1.8 mL/minute. Temperature: 50° C. Detection: UV (214, 4 nm) and MS (ESI, POS mode, 80 to 900 amu).

### LC-MS Method 18:

Mobile phase: A: water (0.01% trifluoroacetic acid), B: acetonitrile (0.01% trifluoroacetic acid); Elution program: Gradient from 5 to 95% of B in 1.2 minutes at 2.0 mL/minute; Column Temperature: 50° C.; Detection: UV (214, 4 nm) and MS (ESI, Pos mode, 132 to 1500 amu).

### LC-MS Method 19:

Mobile Phase: A: water (0.01% trifluoroacetic acid), B: acetonitrile (0.01% trifluoroacetic acid); Gradient: 5% B for 0.2 minutes, increase to 95% B within 3 minutes, 95% B for 2 minutes, back to 5% B within 0.01 minutes; Flow Rate: 1.8 mL/minute; Column: Sunfire, 50×4.6 mm, 3.5 μm; Column Temperature: 50° C.

### LC-MS Method 20:

Mobile phase: A: 10 mM trifluoroacetic acid in water, B: acetonitrile; Gradient: B=5%-95% in 1.5 minutes; Flow rate: 2.0 mL/minute; Column: Xbridge-C18, 50×4.6 mm, 3.5 μm.

### LC-MS Method 21:

Mobile Phase: A: water (0.01% trifluoroacetic acid), B: acetonitrile (0.01% trifluoroacetic acid); Gradient: 5%-95% B in 1.5 minutes; Flow Rate: 1.5 mL/minute; Column: KINETEX C18 5 μm, 3.0×30 mm; Oven Temperature: 50° C.; Mass Range:100-1200.

### LC-MS Method 22:

Mobile phase: A: water (0.01% trifluoroacetic acid), B: acetonitrile (0.01% trifluoroacetic acid); Gradient: B=5%-95% in 1.5 minutes; Flow rate: 2 mL/minute; Column: Sunfire C18, 50×4.6 mm, 3.5 μm.

### LC-MS Method 23:

Mobile Phase: A: water (0.01% trifluoroacetic acid), B: acetonitrile (0.01% trifluoroacetic acid); Gradient: 5% B increase to 95% B within 1.3 minutes, 95% B for 0.7 minutes; Flow Rate: 2.5 mL/minute; Column: Sunshell C18, 30×4.6 mm, 2.6 μm; Column Temperature: 40° C.; Detection: UV (214 nm, 4 nm) and MS (ESI, POS Mode, 110-1300 amu).

### LC-MS Method 24:

Mobile phase: Mobile Phase: A: water (0.1% formic acid), B: Acetonitrile (0.1% formic acid); Gradient:10% B for 0.2 minutes, increase to 90% B within 1.3 minutes, 95% B for 1.5 minutes; Flow Rate: 2 mL/minute; Columns: Sunfire, 50×4.6 mm, 3.5 μm; Column Temperature: 50° C.

### LC-MS Method 25:

Mobile Phase: A: water (0.01% trifluoroacetic acid), B: acetonitrile (0.01% trifluoroacetic acid); Gradient: 5% increase to 95% B within 1.3 minutes, 95% B for 1.7 minutes; Flow Rate: 2 mL/minute; Column: Sunfire, 50×4.6 mm, 3.5 μm; Column Temperature: 50° C.; Detection: UV (214, 4 nm) and MS (ESI, Pos mode, 110 to 1000 amu).

### LC-MS Method 26:

Mobile Phase: A: water (0.01% trifluoroacetic acid), B: acetonitrile (0.01% trifluoroacetic acid); Gradient: 5% B increase to 95% B within 1.3 minutes, 95% B for 1.2 minutes; Flow Rate: 2.2 mL/minute; Column: Chromolith Fast Gradient RP-18e, 50 mm×3 mm.

### LC-MS Method 27:

Mobile phase: Mobile Phase: A: water (10 mM ammonium carbonate), B: acetonitrile; Gradient: 5% B for 0.2 minutes, Increase to 95% B within 1.3 minutes, 95% B for 1.5 minutes, back to 5% within 0.01 minutes; Flow Rate: 2 mL/minute; Column: Sunfire, 50×4.6 mm, 3.5 μm; Oven Temperature: 50° C.

### LC-MS Method 28:

Mobile Phase: A: water (0.01% trifluoroacetic acid), B: acetonitrile (0.01% trifluoroacetic acid); Gradient: 5% increase to 95% B within 1.5 minutes, 95% B for 1.7 minutes; Flow Rate: 2 mL/minute; Column: Sunfire C18, 50×4.6 mm, 3.5 μm; Column Temperature: 50° C.; Detection: UV (214, 4 nm) and MS (ESI, Positive mode, 110 to 1000 amu).

### LC-MS Method 29:

Mobile Phase: A:water (0.01% trifluoroacetic acid), B: acetonitrile (0.01% trifluoroacetic acid) Gradient: 5% B increase to 95% B within 1 minutes, 95% B for 1 minutes. Flow Rate:1.6 mL/minute; Column:AGILENT Poroshell 2.7 um, 3.0 mm*30 mm; Column Temperature:50° C. Detection: UV (214 nm, 4 nm) and MS (ESI, POS Mode, 110-1300 amu).

Preparation of Intermediates

### Intermediate 1: 7-Bromo-5-(3-iodophenyl)-6-oxoheptanenitrile

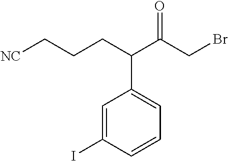 [SMILES: N#CCCCC(C(=O)CBr)c1cccc(I)c1]

To a stirred solution of 5-cyano-2-(3-iodophenyl)pentanoic acid (Intermediate 1A, 5.4 g, 16 mmol) in dichloromethane (20 mL) was added oxalyl chloride (4.2 g, 32 mmol) followed by 3 drops of N,N-dimethylformamide. The reaction was stirred at room temperature for 3 hours and concentrated. The trace of oxalyl was removed by co-evaporate with heptanes twice. The residue was dissolved in tetrahydrofuran (10 mL) and acetonitrile (10 mL). The solution was added dropwise to a solution of (trimethylsilyl) diazomethane (2M in hexane, 32 mL, 64 mmol) at 0° C. The mixture was stirred at room temperature overnight, then re-cooled to 0° C., and treated with hydrogen bromide in acetic acid (33%, 31.4 g, 128 mmol) dropwise. The reaction was stirred for 30 minutes at room temperature and diluted with ethyl acetate (200 mL). The solution was washed with water, saturated sodium bicarbonate and brine, dried over sodium sulfate, and concentrated. The residue was purified by automated flash chromatography (80 g silica gel column, eluting with 0-15% ethyl acetate in petroleum ether) to give the title compound (4.5 g, 68%) as an oil. MS (ESI): 405.9, 407.9 m/z [M+H]⁺, retention time: 2.06 minutes, purity: 90% (214 nm) (LC-MS method 3).

The following intermediates were prepared utilizing the procedures described for Intermediate 1.

| Inter | | | |
|---|---|---|---|
| No. | Structure | Name | MS m/z [M + H]⁺ and/or ¹H NMR |
| 1-1 | 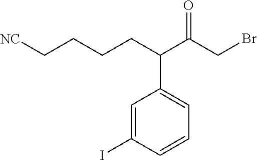 [SMILES: N#CCCCCC(C(=O)CBr)c1cccc(I)c1] | 8-bromo-6-(3- iodophenyl)-7- oxooctanenitrile | 442, 444 [M + Na]⁺ |
| 1-2 | 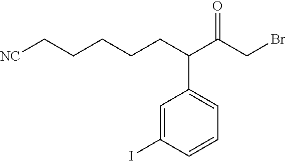 [SMILES: N#CCCCCCC(C(=O)CBr)c1cccc(I)c1] | 9-bromo-7-(3- iodophenyl)-8- oxononanenitrile | 456, 458 [M + Na]⁺ |
| 1-3 | 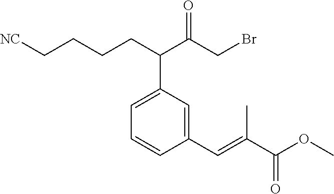 [SMILES: COC(=O)/C(C)=C/c1cccc(C(CCCCC#N)C(=O)CBr)c1] | methyl (E)-3-(3-(1- bromo-7-cyano-2- oxoheptan-3- yl)phenyl)-2- methylacrylate | 392, 394 |
| 1-4 | 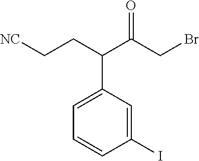 [SMILES: N#CCCC(C(=O)CBr)c1cccc(I)c1] | 6-bromo-4-(3- iodophenyl)-5- oxohexanenitrile | 392, 394 |
| 1-5 | 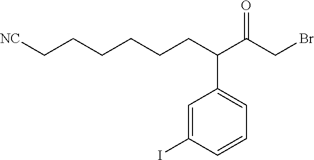 [SMILES: N#CCCCCCCC(C(=O)CBr)c1cccc(I)c1] | 10-bromo-8-(3- iodophenyl)-9- oxodecanenitrile | 470, 472 [M + Na]⁺ |
| 1-6 | 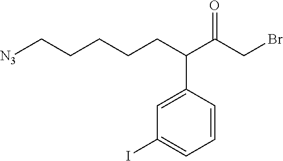 [SMILES: [N-]=[N+]=NCCCCCC(C(=O)CBr)c1cccc(I)c1] | 8-azido-1-bromo-3- (3- iodophenyl)octan-2- one | MS: 472, 474 m/z [M + Na]⁺; ¹H NMR (400 MHz, CDCl₃) δ7.66-7.59 (m, 2H), 7.21-7.19 (m, 1H), 7.12-7.08 (m, 1H), 4.06-4.04 (m, 1H), 3.97-3.95 (m, 1H), 3.79-3.76 (m, 1H), 3.26-3.23 (m, 2H), 2.10-2.04 (m, 1H), 1.75-1.67 (m, 3H), 1.43-1.18 (m, 4H). |
| 1-7 | 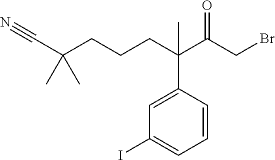 [SMILES: CC(C)(C#N)CCCC(C)(C(=O)CBr)c1cccc(I)c1] | 8-bromo-6-(3- iodophenyl)-2,2,6- trimethyl-7- oxooctanenitrile | MS: 484, 486 m/z [M + Na]⁺; ¹H NMR (400 MHz, CDCl₃) δ 7.65-7.58 (m, 2H), 7.18-7.10 (m, 2H), 3.89-3.75 (m, 2H), 2.06-1.86 (m, 2H), 1.59 (s, 3H), 1.58- 1.34 (m, 4H), 1.31 (s, 3H), 1.28 (s, 3H). |
| 1-8 | 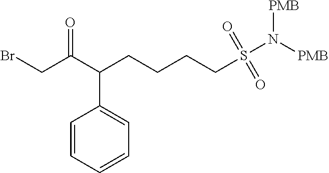 | 7-bromo-N,N-bis(4- methoxybenzyl)-6- oxo-5- phenylheptane-1- sulfonamide | 610, 612 [M + Na]⁺ |
| 1-9 | 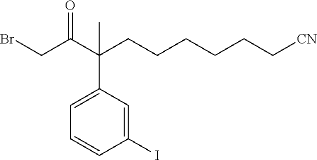 [SMILES: CC(CCCCCCC#N)(C(=O)CBr)c1cccc(I)c1] | 10-bromo-8-(3- iodophenyl)-8- methyl-9- oxodecanenitrile | 484, 486 [M + Na]⁺ |
| 1-10 | 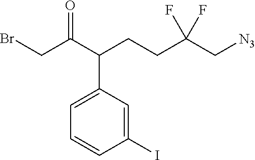 [SMILES: [N-]=[N+]=NCC(F)(F)CCC(C(=O)CBr)c1cccc(I)c1] | 7-azido-1-bromo- 6,6-difluoro-3-(3- iodophenyl)heptan- 2-one | 494, 496 [M + Na]⁺ |
| 1-11 | 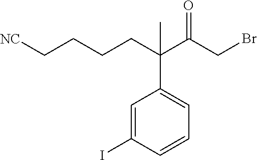 [SMILES: CC(CCCCC#N)(C(=O)CBr)c1cccc(I)c1] | 8-bromo-6-(3- iodophenyl)-6- methyl-7- oxooctanenitrile | 434, 436 |
| 1-12 | 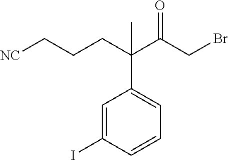 [SMILES: CC(CCCC#N)(C(=O)CBr)c1cccc(I)c1] | 7-bromo-5-(3- iodophenyl)-5- methyl-6- oxoheptanenitrile | 420, 422 |
| 1-13* | 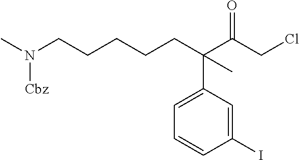 [SMILES: CN(CCCCCC(C)(C(=O)CCl)c1cccc(I)c1)C(=O)OCc1ccccc1] | benzyl (8-chloro-6- (3-iodophenyl)-6- methyl-7- oxooctyl)(methyl) carbamate | 542 |
| 1-14* | 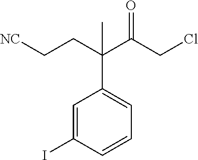 [SMILES: CC(CCC#N)(C(=O)CCl)c1cccc(I)c1] | 6-chloro-4-(3- iodophenyl)-4- methyl-5- oxohexanenitrile | Mass: 362 m/z [M + H]⁺, ¹HNMR (400 MHz, CDCl₃): δ 7.72 (d, J = 6.8 Hz, 1H), 7.55 (s, 1H), 7.18-7.12 (m, 2H), 4.13- 3.89 (m, 2H), 2.31-2.26 (m, 2H), 2.18- 2.11 (m, 2H), 1.64 (s, 3H) ppm. |
| 1-15 | 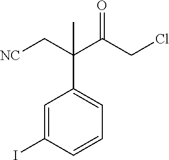 [SMILES: CC(CC#N)(C(=O)CCl)c1cccc(I)c1] | 5-chloro-3-(3- iodophenyl)-3- methyl-4- oxopentanenitrile | MS: 348 m/z [M + H]⁺. ¹HNMR (400 MHz, CDCl₃): δ 7.74-7.76 (m, 1H), 7.60 (s, 1H), 7.16-7.22 (m, 2H), 3.92-4.17 (m, 2H), 2.83-2.99 (m, 2H), 1.822 (s, 3H) ppm. |
| 1-16* | 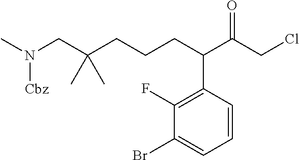 | benzyl (6-(3-bromo- 2-fluorophenyl)-8- chloro-2,2-dimethyl- 7- oxooctyl)(methyl) carbamate | MS: 550 m/z [M + Na]⁺; ¹H NMR (400 MHz, CDCl₃) δ 7.50-7.47 (m, 1H), 7.35- 7.28 (m, 5H), 7.16-7.08 (m, 1H), 7.04- 7.00 (m, 1H), 5.12-5.08 (m, 2H), 4.29- 4.04 (m, 3H), 3.03-3.13 (m, 2H), 2.95 (s, 3H), 2.06-2.01 (m, 1H), 1.67-1.62 (m, 1H), 1.29-1.12 (m, 4H), 0.88-0.79 (m, 6H) ppm. |
| 1-17 | 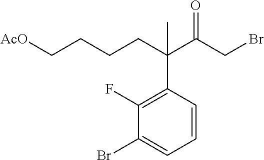 [SMILES: CC(=O)OCCCCC(C)(C(=O)CBr)c1cccc(Br)c1F] | 7-bromo-5-(3- bromo-2 fluorophenyl)-5- methyl-6-oxoheptyl acetate | 461 [M + Na]⁺ |
| 1-18 | 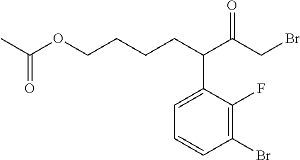 [SMILES: CC(=O)OCCCCC(C(=O)CBr)c1cccc(Br)c1F] | 7-bromo-5-(3- bromo-2- fluorophenyl)-6- oxoheptyl acetate | 447 [M + Na]⁺ |
| 1-19 | 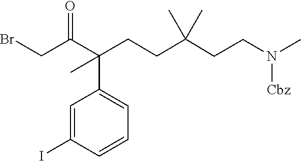 [SMILES: CN(CCC(C)(C)CCC(C)(C(=O)CBr)c1cccc(I)c1)C(=O)OCc1ccccc1] | benzyl (8-bromo-6- (3-iodophenyl)- 3,3,6-trimethyl-7- oxooctyl)(methyl) carbamate | 636, 638 [M + Na]⁺ |
| 1-20 | 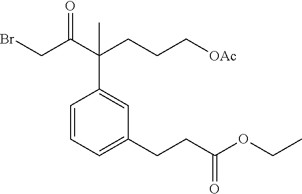 [SMILES: CCOC(=O)CCc1cccc(C(C)(CCCOC(C)=O)C(=O)CBr)c1] | ethyl 3-(3-(6- acetoxy-1-bromo-3- methyl-2-oxohexan- 3- yl)phenyl)propanoate | 427, 429 |
| 1-21 | 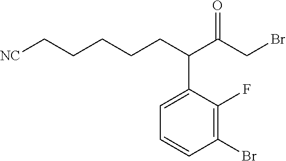 [SMILES: N#CCCCCCC(C(=O)CBr)c1cccc(Br)c1F] | 9-bromo-7-(3- bromo-2- fluorophenyl)-8- oxononanenitrile | 406 [M + Na]⁺ |
| 1-22 | 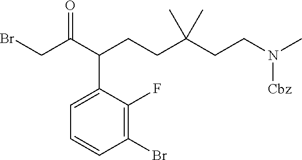 [SMILES: CN(CCC(C)(C)CCC(C(=O)CBr)c1cccc(Br)c1F)C(=O)OCc1ccccc1] | benzyl (8-bromo-6- (3-bromo-2- fluorophenyl)-3,3- dimethyl-7- oxooctyl)(methyl) carbamate | 594 [M + Na]⁺ |
| 1-23 | 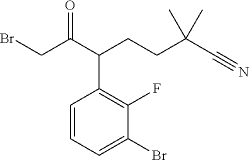 [SMILES: CC(C)(C#N)CCC(C(=O)CBr)c1cccc(Br)c1F] | 7-bromo-5-(3- bromo-2- fluorophenyl)-2,2- dimethyl-6- oxoheptanenitrile | MS: 406 m/z [M + H]⁺; ¹H NMR (400 MHz, CDCl₃) δ 7.55-7.51 (m, 1H), 7.20- 7.01 (m, 2H), 4.41 (t, J = 7.3 Hz, 1H), 3.94-3.80 (m, 2H), 2.38-2.18 (m, 1H), 1.95-1.81 (m, 1H), 1.58-1.39 (m, 2H), 1.35 (s, 3H), 1.33 (s, 3H) ppm |
| 1-24 | 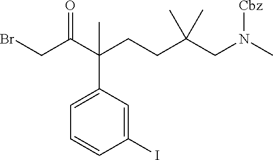 [SMILES: CN(CC(C)(C)CCC(C)(C(=O)CBr)c1cccc(I)c1)C(=O)OCc1ccccc1] | benzyl (7-bromo-5- (3-iodophenyl)- 2,2,5-trimethyl-6- oxoheptyl)(methyl) carbamate | 600, 602 |
| 1-25 | 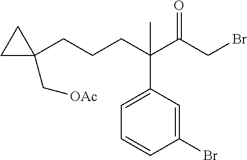 [SMILES: CC(=O)OCC1(CCCC(C)(C(=O)CBr)c2cccc(Br)c2)CC1] | (1-(6-bromo-4-(3- bromophenyl)-4- methyl-5- oxohexyl)cycloprop yl)methyl acetate | 483 [M + Na]⁺ |
| 1-26 | 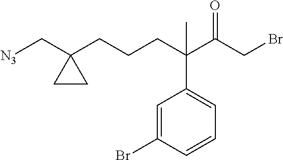 [SMILES: CC(CCCC1(CN=[N+]=[N-])CC1)(C(=O)CBr)c1cccc(Br)c1] | 6-(1- (azidomethyl)cyclo- propyl)-1-bromo-3- (3-bromophenyl)-3- methylhexan-2-one | 466 [M + Na]⁺ |
| 1-27 | 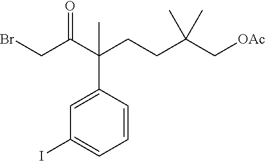 [SMILES: CC(=O)OCC(C)(C)CCC(C)(C(=O)CBr)c1cccc(I)c1] | 7-bromo-5-(3- iodophenyl)-2,2,5- trimethyl-6- oxoheptyl acetate | 495, 497 |
| 1-28 | 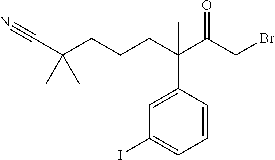 [SMILES: CC(C)(C#N)CCCC(C)(C(=O)CBr)c1cccc(I)c1] | 8-bromo-6-(3- iodophenyl)-2,2,6- trimethyl-7- oxooctanenitrile | Mass: 462, 464 [M + H]⁺. ¹H NMR (400 MHz, CD₃OD) 8 7.73-7.69 (m, 1H), 7.66 (t, J = 1.7 Hz, 1H), 7.33-7.29 (m, 1H), 7.19 (t, J = 7.9 Hz, 1H), 4.14-3.95 (m, 2H), 2.04-1.91 (m, 2H), 1.62-1.58 (m, 3H), 1.59-1.49 (m, 2H), 1.38-1.23 (m, 8H). |
| 1-29* | 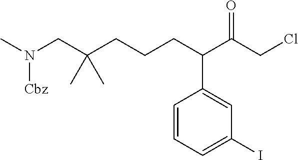 [SMILES: CN(CC(C)(C)CCCC(C(=O)CCl)c1cccc(I)c1)C(=O)OCc1ccccc1] | benzyl (8-chloro-6- (3-iodophenyl)-2,2- dimethyl-7- oxooctyl)(methyl) carbamate | 578 [M + Na]⁺ |
| 1-30 | 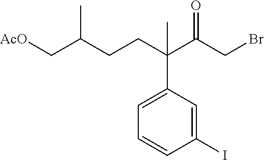 [SMILES: CC(=O)OCC(C)CCC(C)(C(=O)CBr)c1cccc(I)c1] | 7-bromo-5-(3- iodophenyl)-2,5- dimethyl-6- oxoheptyl acetate | 481, 483 |
| 1-31 | 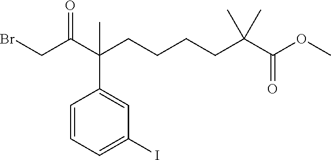 [SMILES: COC(=O)C(C)(C)CCCCC(C)(C(=O)CBr)c1cccc(I)c1] | methyl 9-bromo-7- (3-iodophenyl)- 2,2,7-trimethyl-8- oxononanoate | 531, 533 [M + Na]⁺ |
| 1-32 | 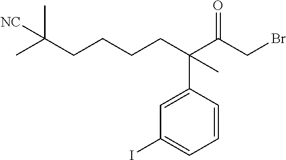 [SMILES: CC(C)(C#N)CCCCC(C)(C(=O)CBr)c1cccc(I)c1] | 9-bromo-7-(3- iodophenyl)-2,2,7- trimethyl-8- oxononanenitrile | 498, 500 [M + Na]⁺ |
| 1-33 | 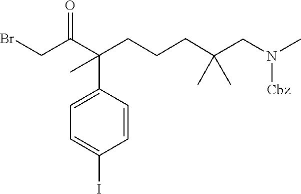 [SMILES: CN(CC(C)(C)CCCC(C)(C(=O)CBr)c1ccc(I)cc1)C(=O)OCc1ccccc1] | benzyl (8-bromo-6- (4-iodophenyl)- 2,2,6-trimethyl-7- oxooctyl)(methyl) carbamate | 636, 638 [M + Na]⁺ |
| 1-34 | 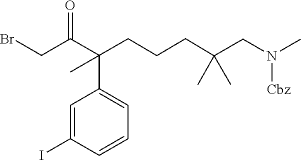 [SMILES: CN(CC(C)(C)CCCC(C)(C(=O)CBr)c1cccc(I)c1)C(=O)OCc1ccccc1] | benzyl (8-bromo-6- (3-iodophenyl)- 2,2,6-trimethyl-7- oxooctyl)(methyl) carbamate | MS: 636, 638 [M + Na]⁺; ¹H NMR (400 MHz, CDCl₃) δ 7.65-7.55 (m, 2H), 7.36- 7.28 (m, 5H), 7.15-7.07 (m, 2H), 5.11 (s, 2H), 3.90-3.72 (m, 2H),3.17-2.98 (m, 2H),2.95 (s, 3H), 1.90-1.73 (m, 2H), 1.49 (s, 3H), 1.27-1.01 (m, 4H), 0.85-0.79 (m, 6H) ppm. |
| 1-35 | 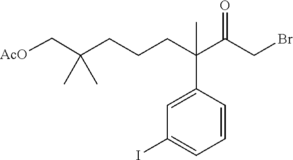 [SMILES: CC(=O)OCC(C)(C)CCCC(C)(C(=O)CBr)c1cccc(I)c1] | 8-bromo-6-(3- iodophenyl)-2,2,6- trimethyl-7-oxooctyl acetate | 531, 533 [M + Na]⁺ |
| 1-36 | 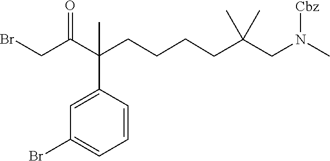 [SMILES: CN(CC(C)(C)CCCCC(C)(C(=O)CBr)c1cccc(Br)c1)C(=O)OCc1ccccc1] | benzyl (9-bromo-7- (3-bromophenyl)- 2,2,7-trimethyl-8- oxononyl)(methyl) carbamate | 604 [M + Na]⁺ |
| 1-37 | 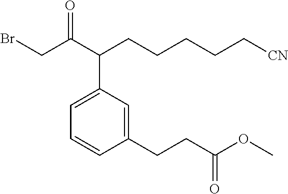 [SMILES: COC(=O)CCc1cccc(C(CCCCCC#N)C(=O)CBr)c1] | methyl 3-(3-(1- bromo-8-cyano-2- oxooctan-3- yl)phenyl)propanoate | 394, 396 |
| 1-38 | 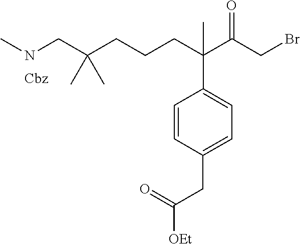 [SMILES: CCOC(=O)Cc1ccc(C(C)(CCCC(C)(C)CNC)C(=O)CBr)cc1 ; "Cbz"] | ethyl 2-(4-(8- (((benzyloxy) carbonyl) (methyl)amino)- 1-bromo-3,7,7- trimethyl-2- oxooctan-3- yl)phenyl)acetate | 574, 576 |
| 1-39 | 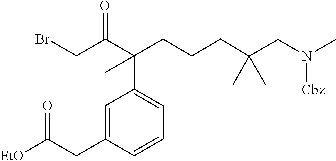 [SMILES: CCOC(=O)Cc1cccc(C(C)(CCCC(C)(C)CN(C)C(=O)OCc2ccccc2)C(=O)CBr)c1] | ethyl 2-(3-(8- (((benzyloxy) carbonyl) (methyl)amino)- 1-bromo-3,7,7- trimethyl-2- oxooctan-3- yl)phenyl)acetate | 596, 598 [M + Na]⁺ |
| 1-40* | 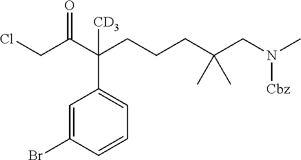 [SMILES: [2H]C([2H])([2H])C(CCCC(C)(C)CN(C)C(=O)OCc1ccccc1)(C(=O)CCl)c1cccc(Br)c1] | benzyl (6-(3- bromophenyl)-8- chloro-2,2-dimethyl- 6-(methyl-d3)-7- oxooctyl)(methyl) carbamate | 549 [M + Na]⁺ |
| 1-41 | 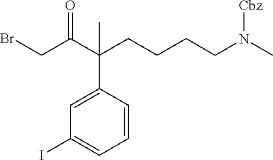 [SMILES: CN(CCCCC(C)(C(=O)CBr)c1cccc(I)c1)C(=O)OCc1ccccc1] | benzyl (7-bromo-5- (3-iodophenyl)-5- methyl-6- oxoheptyl)(methyl) carbamate | 572, 574 |
| 1-42 | 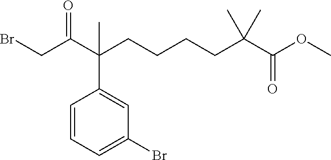 [SMILES: COC(=O)C(C)(C)CCCCC(C)(C(=O)CBr)c1cccc(Br)c1] | methyl 9-bromo-7- (3-bromophenyl)- 2,2,7-trimethyl-8- oxononanoate | 485 [M + Na]⁺ |
| 1-43 | 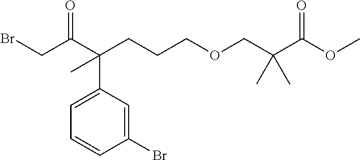 [SMILES: COC(=O)C(C)(C)COCCCC(C)(C(=O)CBr)c1cccc(Br)c1] | methyl 3-((6-bromo- 4-(3-bromophenyl)- 4-methyl-5- oxohexyl)oxy)-2,2- dimethylpropanoate | 501 [M + Na]⁺ |
| 1-44* | 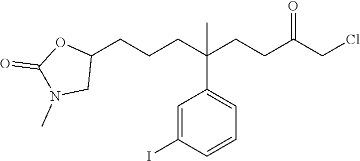 [SMILES: CN1CC(CCCC(C)(CCC(=O)CCl)c2cccc(I)c2)OC1=O] | 5-(6-chloro-4-(3- iodophenyl)-4- methyl-5-oxohexyl)- 3-methyloxazolidin- 2-one | 450 |
| 1-45 | 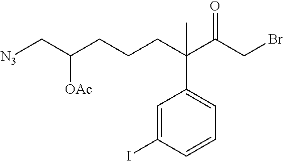 [SMILES: CC(=O)OC(CCCC(C)(C(=O)CBr)c1cccc(I)c1)CN=[N+]=[N-]] | 1-azido-8-bromo-6- (3-iodophenyl)-6- methyl-7-oxooctan- 2-yl acetate | 522, 524 |
| 1-46 | 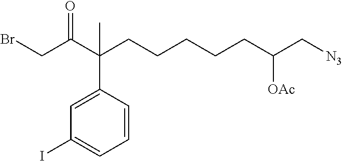 [SMILES: CC(=O)OC(CCCCCC(C)(C(=O)CBr)c1cccc(I)c1)CN=[N+]=[N-]] | 1-azido-10-bromo- 8-(3-iodophenyl)-8- methyl-9-oxodecan- 2-yl acetate | 550, 552 |
| 1-47 | 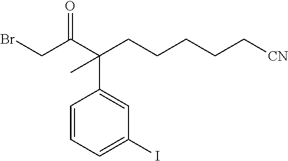 [SMILES: CC(CCCCCC#N)(C(=O)CBr)c1cccc(I)c1] | 9-bromo-7-(3- iodophenyl)-7- methyl-8- oxononanenitrile | 448, 450 |
| 1-48* | 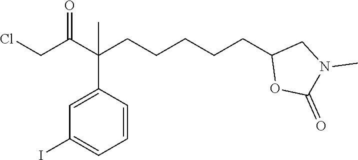 [SMILES: CN1CC(CCCCCC(C)(C(=O)CCl)c2cccc(I)c2)OC1=O] | 5-(8-chloro-6-(3- iodophenyl)-6- methyl-7-oxooctyl)- 3-methyloxazolidin- 2-one | 478 |
| 1-49 | 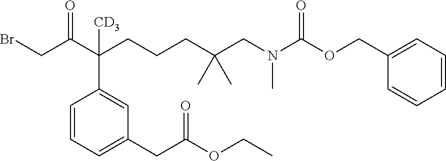 [SMILES: [2H]C([2H])([2H])C(CCCC(C)(C)CN(C)C(=O)OCc1ccccc1)(C(=O)CBr)c1cccc(CC(=O)OCC)c1] | ethyl 2-(3-(8- (((benzyloxy) carbonyl) (methyl)amino)- 1-bromo-7,7- dimethyl-3-(methyl- d3)-2-oxooctan-3- yl)phenyl)acetate | 599, 581 [M + Na]⁺ |

*Quenching diazomethylketone with hydrogen chloride in 1,4-dioxane afforded the chloroketone

### Intermediate 1A: 5-Cyano-2-(3-iodophenyl)pentanoic acid

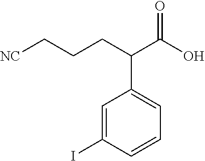 [SMILES: N#CCCCC(C(=O)O)c1cccc(I)c1]

To a stirred and cooled (−78° C.) solution of 2-(3-iodophenyl)acetic acid (5 g, 19 mmol) in tetrahydrofuran (100 mL) was added lithium diisopropylamide (2 M in tetrahydrofuran, 29 mL, 58 mmol). The mixture was stirred at −78° C. for 1 hour, then treated with 4-bromobutanenitrile (9 g, 60.8 mmol). The reaction was stirred at room temperature overnight, quenched with water (100 mL), and pH adjusted to ˜5-6 with 2 N hydrochloric acid. The solution was extracted with ethyl acetate (3×60 mL). The combined organic layers were washed with brine, dried over sodium sulfate, and concentrated. The residue was purified by silica gel column chromatography (eluting with dichloromethane/methanol 0-24%) to give the title compound (4.5 g, 70%) as a brown oil. MS (ESI): 352 m/z [M+Na]⁺, retention time: 1.85 minutes, purity: 90% (214 nm) (LC-MS method 4).

The following intermediates were prepared utilizing the procedures described for Intermediate 1A.

| Inter | | | MS m/z [M + H]⁺ and/or |
|---|---|---|---|
| No. | Structure | Name | ¹H NMR |
| 1A-1 | 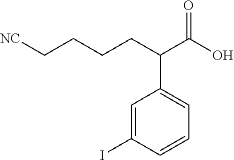 [SMILES: N#CCCCCC(C(=O)O)c1cccc(I)c1] | 6-cyano-2-(3- iodophenyl) hexanoic acid | 366 [M + Na]⁺ |
| 1A-2 | 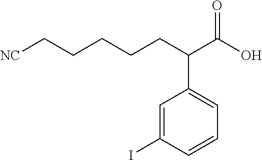 [SMILES: N#CCCCCCC(C(=O)O)c1cccc(I)c1] | 7-cyano-2-(3- iodophenyl) heptanoic acid | 380 [M + Na]⁺ |
| 1A-3 | 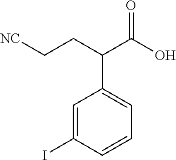 [SMILES: N#CCCC(C(=O)O)c1cccc(I)c1] | 4-cyano-2-(3- iodophenyl) butanoic acid | 338 [M + Na]⁺ |
| 1A-4 | 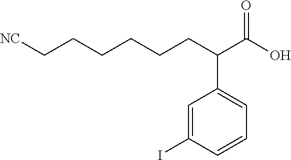 [SMILES: N#CCCCCCCC(C(=O)O)c1cccc(I)c1] | 8-cyano-2-(3- iodophenyl) octanoic acid | 372 |
| 1A-5 | 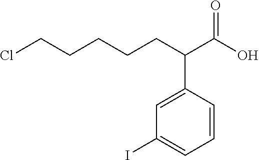 [SMILES: O=C(O)C(CCCCCCl)c1cccc(I)c1] | 7-chloro-2-(3- iodophenyl) heptanoic acid | MS: 367 [M + H]⁺; ¹H NMR (400 MHz, CDCl₃) δ 7.68-7.62 (m, 2H), 7.32-7.30 (d, 1H), 7.10-7.06 (m, 1H), 3.55-3.43 (m, 3H), 2.13-1.72 (m, 4H), 1.51-1.25 (m, 4H). |
| 1A-6 | 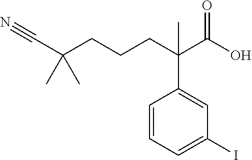 [SMILES: CC(C)(C#N)CCCC(C)(C(=O)O)c1cccc(I)c1] | 6-cyano-2-(3- iodophenyl)-2,6- dimethylheptanoic acid | MS: 408 [M + Na]⁺; ¹H NMR (400 MHz, CDCl₃) δ 7.71-7.70 (m, 1H), 7.63- 7.61(m, 1H), 7.36-7.34 (m, 1H), 7.12- 7.08 (m, 1H), 2.06-1.90 (m, 2H), 1.60 (s, 3H), 1.55-1.37 (m, 4H), 1.33 (s, 3H), 1.31 (s, 3H). |
| 1A-7 | 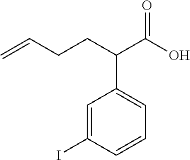 | 2-(3- iodophenyl)hex-5- enoic acid | 317 |
| 1A-8 | 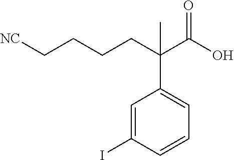 [SMILES: CC(CCCCC#N)(C(=O)O)c1cccc(I)c1] | 6-cyano-2-(3- iodophenyl)-2- methylhexanoic acid | 375 [M + NH3 + H]⁺ |
| 1A-9 | 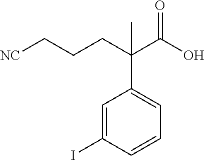 [SMILES: CC(CCCC#N)(C(=O)O)c1cccc(I)c1] | 5-cyano-2-(3- iodophenyl)-2- methylpentanoic acid | 344 |
| 1A-10 | 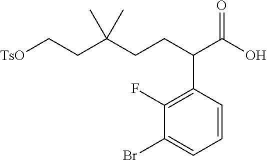 [SMILES: Cc1ccc(S(=O)(=O)OCCC(C)(C)CCC(C(=O)O)c2cccc(Br)c2F)cc1] | 2-(3-bromo-2- fluorophenyl)-5,5- dimethyl-7- (tosyloxy)heptanoic acid | 518, 520 [M + NH3 + H]⁺ |
| 1A-11 | 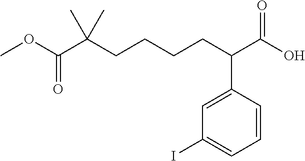 [SMILES: COC(=O)C(C)(C)CCCCC(C(=O)O)c1cccc(I)c1] | 2-(3-iodophenyl)-8- methoxy-2,7,7- trimethyl-8- oxooctanoic acid | 455 [M + Na]⁺ |
| 1A-12 | 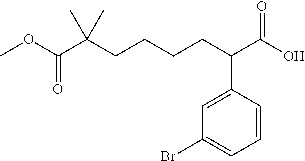 [SMILES: COC(=O)C(C)(C)CCCCC(C(=O)O)c1cccc(Br)c1] | 2-(3-bromophenyl)- 8-methoxy-2,7,7- trimethyl-8- oxooctanoic acid | 407, 409 [M + Na]⁺ |

### Intermediate 2: 5-((4-Bromo-6-fluoro-1H-indol-5-yl)oxy)-2-fluorobenzonitrile

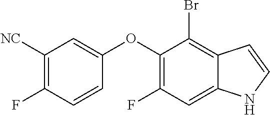 [SMILES: N#Cc1cc(Oc2c(F)cc3[nH]ccc3c2Br)ccc1F]

Step One: To a stirred solution of Intermediate 2B (124 g, 336 mmol) in N,N-dimethylformamide (1 L) was added N,N-dimethylformamide dimethyl acetal (178 mL, 159 g, 1.34 mol). Five identical reactions were executed in parallel. The six mixtures were each heated at 100° C. for six hours and then cooled to room temperature, combined, and poured into stirred ice water (20 L). After warming to near room temperature, the suspension was extracted with ethyl acetate (2×8 L). The combined organic layers were washed with water (1×10 L) and brine (1×10 L), dried over sodium sulfate and concentrated. The crude N,N-dimethyl enamine intermediate, which was used without purification in the second step, was afforded as a black oil (786 g, 92%). ¹H NMR (400 MHz CDCl₃) δ 7.38 (d, J=8.8 Hz, 1H), 7.19 (s, 1H), 7.10-7.12 (m, 2H), 6.44 (d, J=13.6 Hz, 1H), 4.96 (d, J=13.6 Hz, 1H), 2.80 (s, 6H) ppm.

Step Two: To a stirred solution of the crude enamine (100 g, 236 mmol) in a mixture of acetic acid (800 mL) and toluene (800 mL) was added silica gel (42.5 g). The suspension was warmed to 50° C. and treated with iron powder (132 g, 2.36 mol) portion-wise over 15 minutes.

Following this addition, the mixture was heated at 100° C. for 12 hours and then cooled to room temperature and suction filtered through a bed of Celite. The filtering agent was rinsed with ethyl acetate (total 5 L) and the combined filtrate was partitioned between water (10 L) and ethyl acetate (5 L). The organic layer was combined with a second extract (ethyl acetate, 1×5 L), washed with water (1×10 L) and brine (1×10 L), dried over sodium sulfate and concentrated under reduced pressure to give a dark brown oil. The resulting dark brown oil was purified by automated flash chromatography (1 kg silica gel column, 1-20% ethyl acetate in petroleum ether) to afford the title compound as a white solid (66.7 g, 74% overall, two steps). ¹H NMR (400 MHz CDCl₃) δ 8.41 (s, 1H), 7.25-7.26 (m, 1H), 7.15-7.18 (m, 3H), 6.95-7.09 (m, 1H), 6.55 (t, J=2.8 Hz, 1H) ppm.

The following intermediates were prepared utilizing the procedure described for Intermediate 2.

| Inter. | | | MS m/z [M + H]⁺/ |
|---|---|---|---|
| No. | Structure | Name | ¹H NMR |
| 2-1 | 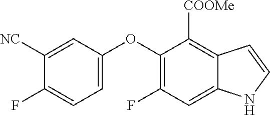 [SMILES: COC(=O)c1c(Oc2ccc(F)c(C#N)c2)c(F)cc2[nH]ccc12] | methyl 5-(3-cyano-4- fluorophenoxy)-6-fluoro- 1H-indole-4-carboxylate | 329 |
| 2-2 | 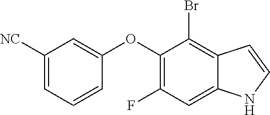 [SMILES: N#Cc1cccc(Oc2c(F)cc3[nH]ccc3c2Br)c1] | 3-((4-bromo-6-fluoro- 1H-indol-5- yl)oxy)benzonitrile | 331, 333 |

### Intermediate 2A: 3-Bromo-1,2-difluoro-4-methyl-5-nitrobenzene

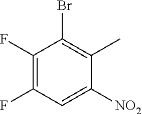

To a stirred solution of 1,2-difluoro-4-methyl-5-nitrobenzene (150 g, 866 mmol) in trifluoroacetic acid (800 mL) was added 1,3-dibromo-5,5-dimethylimidazolidine-2,4-dione (136 g, 476 mmol) and concentrated sulfuric acid (200 mL; over 3-4 minutes). Two additional bromination reactions, utilizing the same quantities of reactants and solvent, were run in parallel. After 10 hours at room temperature, the three reactions were combined and then slowly poured into a stirred slurry of crushed ice and water (5 L). When the ice had fully melted, the mixture was extracted with petroleum ether (2×4 L). The combined organic layers were washed with brine (1×5 L), dried over sodium sulfate and concentrated. The resulting oil was purified by automated flash chromatography (3 kg silica gel column, 100% petroleum ether) to afford the title compound as a yellow oil (417 g, 64%). ¹H NMR (400 MHz CDCl₃) δ 7.70-7.64 (m, 1H), 2.55 (s, 3H) ppm.

### Intermediate 2B: 5-(2-Bromo-6-fluoro-3-methyl-4-nitrophenoxy)-2-fluorobenzonitrile

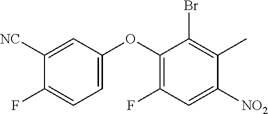 [SMILES: Cc1c([N+](=O)[O-])cc(F)c(Oc2ccc(F)c(C#N)c2)c1Br]

To a stirred solution of Intermediate 2A (207 g, 820 mmol) in N,N-dimethylformamide (1 L) was added 2-fluoro-5-hydroxybenzonitrile (118 g, 861 mmol) and potassium carbonate (227 g, 1.64 mol). A second, identical, reaction was run in parallel. Both mixtures were heated at 100° C. for one hour and then cooled to room temperature, combined, and poured into stirred ice water (7 L). After warming to room temperature, the resulting suspension was extracted with ethyl acetate (2×3 L). The combined organic layers were washed with water (1×5 L) and brine (1×3 L), dried over sodium sulfate and concentrated. The crude title compound, which was used without purification, was afforded as a yellow solid (585 g, 97%). ¹H NMR (400 MHz CDCl₃) δ 7.68 (d, J=9.2 Hz, 1H), 7.10-7.14 (m, 2H), 7.01-7.02 (m, 1H), 2.58 (s, 3H).

### Intermediate 3: 5-((4-Bromo-6-fluoro-1-tosyl-1H-indol-5-yl)oxy)-2-fluorobenzonitrile

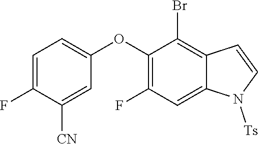 [SMILES: Cc1ccc(S(=O)(=O)n2ccc3c(Br)c(Oc4ccc(F)c(C#N)c4)c(F)cc32)cc1]

To a solution of 5-((4-bromo-6-fluoro-1H-indol-5-yl)oxy)-2-fluorobenzonitrile (Intermediate 2, 72 g, 264 mmol) in N,N-dimethylformamide (500 mL) was carefully added sodium hydride (12.7 g, 317 mmol, 60% in mineral oil) at room temperature. The reaction mixture was stirred for 30 minutes, 4-methylbenzenesulfonyl chloride (47.2 g, 247 mmol) was then added portion wise and continued stirring for 9.5 hours. The reaction was quenched with water (3 L) and extracted with ethyl acetate (2 L×2). The combined organic extracts were washed with brine (2 L), dried over sodium sulfate, and concentrated to afford the title compound as a white solid (144 g). ¹H NMR: (400 MHz CDCl₃) δ 7.88-7.84 (m, 1H), 7.79 (d, J=8.0 Hz, 2H), 7.67 (d, J=2.8 Hz, 1H), 7.31 (d, J=8.0 Hz, 2H), 7.16-7.12 (m, 2H), 6.98 (s, 1H), 6.72 (d, J=2.8 Hz, 1H), 2.39 (s, 3H) ppm.

The following intermediates were prepared utilizing the procedure described for Intermediate 3.

| Inter | | | MS m/z [M + H]⁺ and/or |
|---|---|---|---|
| No. | Structure | Name | ¹H NMR |
| 3-1 | 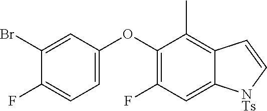 [SMILES: Cc1ccc(S(=O)(=O)n2ccc3c(C)c(Oc4ccc(F)c(Br)c4)c(F)cc32)cc1] | 5-(3-bromo-4- fluorophenoxy)-6- fluoro-4-methyl-1- tosyl-1H-indole | (400 MHz, CDCl₃) 7.80-7.77 (m, 2H), 7.70-7.67 (m, 1H), 7.61-7.58 (m, 1H), 7.31-7.27 (m, 2H), 6.96-6.03 (m, 2H), 6.73-6.33 (m, 1H), 6.68-6.64 (m, 1H), 2.39 (s, 3H), 2.30 (s, 3H) ppm. |
| 3-2 | 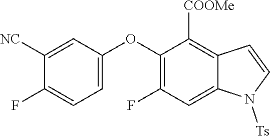 [SMILES: COC(=O)c1c(Oc2ccc(F)c(C#N)c2)c(F)cc2c1ccn2S(=O)(=O)c1ccc(C)cc1] | methyl 5-(3-cyano- 4-fluorophenoxy)-6- fluoro-1-tosyl-1H- indole-4-carboxylate | 483 |
| 3-3 | 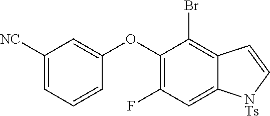 [SMILES: Cc1ccc(S(=O)(=O)n2ccc3c(Br)c(Oc4cccc(C#N)c4)c(F)cc32)cc1] | 3-((4-bromo-6- fluoro-1-tosyl-1H- indol-5- yl)oxy)benzonitrile | 485, 487 |
| 3-4 | 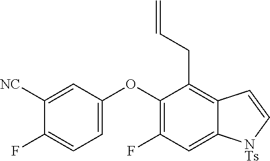 [SMILES: C=CCc1c(Oc2ccc(F)c(C#N)c2)c(F)cc2c1ccn2S(=O)(=O)c1ccc(C)cc1] | 5-((4-allyl-6-fluoro- 1-tosyl-1H-indol-5- yl)oxy)-2- fluorobenzonitrile | ¹H NMR (400 MHz, DMSO-d₆) δ 8.02- 7.97 (m, 2H), 7.92-7.89 (m, 1H), 7.85- 7.81 (m, 1H), 7.53-7.49 (m, 1H), 7.48- 7.42 (m, 3H), 7.31-7.25 (m, 1H), 7.02- 6.96 (m, 1H), 5.77-5.73 (m, 1H), 4.95- 4.91 (m, 2H), 3.53-3.48 (m, 2H), 2.36 (s, 3 H). |
| 3-5 | 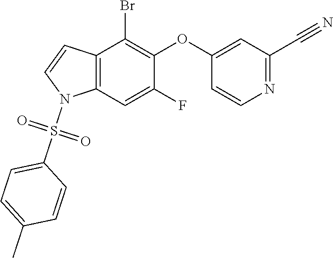 [SMILES: Cc1ccc(S(=O)(=O)n2ccc3c(Br)c(Oc4ccnc(C#N)c4)c(F)cc32)cc1] | 4-((4-bromo-6- fluoro-1-tosyl-1H- indol-5- yl)oxy)picolinonitrile | 486, 488 |

### Intermediate 4: Methyl (E)-3-(5-(3-cyano-4-fluorophenoxy)-6-fluoro-IH-indol-4-yl)acrylate

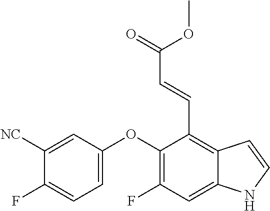 [SMILES: COC(=O)/C=C/c1c(Oc2ccc(F)c(C#N)c2)c(F)cc2[nH]ccc12]

To a stirred and degassed solution of 5-((4-bromo-6-fluoro-1H-indol-5-yl)oxy)-2-fluorobenzonitrile (Intermediate 2, 4 g, 12 mmol) in N,N-dimethylformamide (40 mL) was added methyl acrylate (2.4 g, 24 mmol), triethylamine (8 mL, 60 mmol), tri(o-tolyl)phosphine (800 mg, 2.4 mmol) and palladium(II) acetate (272 mg, 1.2 mmol). The reaction mixture was heated at 110° C. for 4 hours in a microwave reactor. The mixture was cooled to room temperature, diluted with water (50 mL), and extracted with ethyl acetate (3×60 mL). The combined organic extracts were washed with water, brine, dried over sodium sulfate, and concentrated. The residue was purified by automated flash chromatography (80 g silica gel column, eluting with 0-35% ethyl acetate in petroleum ether) to give the title compound (2.1 g, 48%) as a yellow solid. MS (ESI): 355 m/z [M+H]⁺, retention time: 1.86 minutes, purity: 80% (214 nm) (LC-MS method 4).

The following intermediates were prepared utilizing the procedure described for Intermediate 4.

| Inter | | | MS m/z [M + H]⁺ and/or |
|---|---|---|---|
| No. | Structure | Name | ¹H NMR |
| 4-1 | 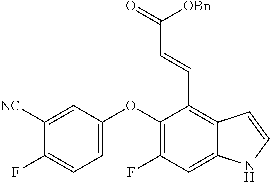 [SMILES: N#Cc1cc(Oc2c(F)cc3[nH]ccc3c2/C=C/C(=O)OCc2ccccc2)ccc1F] | benzyl (E)-3-(5-(3-cyano-4- fluorophenoxy)-6-fluoro-1H- indol-4-yl)acrylate | 431 |
| 4-2 | 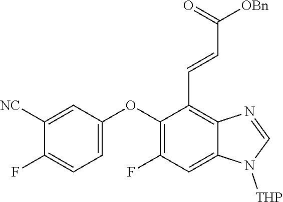 | benzyl (E)-3-(5-(3-cyano-4- fluorophenoxy)-6-fluoro-1- (tetrahydro-2H-pyran-2-yl)- 1H-benzo[d]imidazol-4- yl)acrylate | 516 |
| 4-3 | 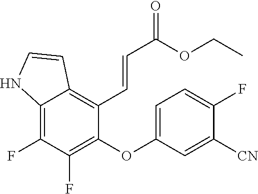 [SMILES: CCOC(=O)/C=C/c1c(Oc2ccc(F)c(C#N)c2)c(F)c(F)c2[nH]ccc12] | ethyl (E)-3-(5-(3-cyano-4- fluorophenoxy)-6,7-difluoro- 1H-indol-4-yl)acrylate | 387 |
| 4-4 | 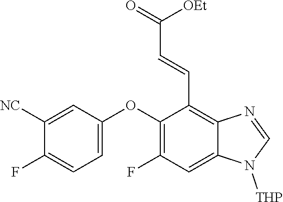 | ethyl (E)-3-(5-(3-cyano-4- fluorophenoxy)-6-fluoro-1- (tetrahydro-2H-pyran-2-yl)- 1H-benzo[d]imidazol-4- yl)acrylate | 454 |
| 4-5 | 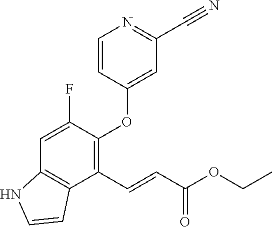 [SMILES: CCOC(=O)/C=C/c1c(Oc2ccnc(C#N)c2)c(F)cc2[nH]ccc12] | ethyl (E)-3-(5-((2- cyanopyridin-4-yl)oxy)-6- fluoro-1H-indol-4-yl)acrylate | 352 |

### Intermediate 5: Methyl (E)-3-(5-(3-carbamimidoyl-4-fluorophenoxy)-6-fluoro-H-indol-4-yl)acrylate

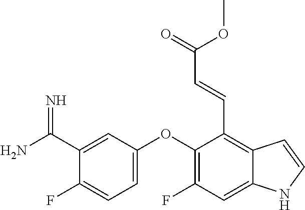 [SMILES: COC(=O)/C=C/c1c(Oc2ccc(F)c(C(=N)N)c2)c(F)cc2[nH]ccc12]

To a stirred and cooled (0° C.) solution of methyl (E)-3-(5-(3-cyano-4-fluorophenoxy)-6-fluoro-1H-indol-4-yl)acrylate (Intermediate 4, 1.6 g, 4.5 mmol) in tetrahydrofuran (36 mL) was added lithium bis(trimethylsilyl)amide (1M in tetrahydrofuran, 36 mL, 36 mmol). The mixture was stirred at 0° C. for 4 hours, then at room temperature overnight, and quenched with water (100 mL). The solution was extracted with dichloromethane (5×60 mL). The combined organic extracts were dried over sodium sulfate and concentrated to give the crude title compound (1.4 g, 83%) as a yellow solid, which was used for the next step without further purification. MS (ESI): 372 m/z [M+H]⁺, retention time: 1.29 minutes, purity: 87% (254 nm) (LC-MS method 3).

The following intermediates were prepared utilizing the procedure described for Intermediate 4.

| Inter | | | MS m/z [M + H]⁺ and/or |
|---|---|---|---|
| No. | Structure | Name | ¹H NMR |
| 5-1 | 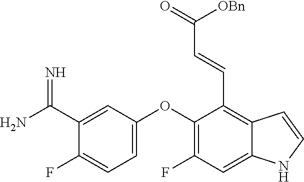 [SMILES: N=C(N)c1cc(Oc2c(F)cc3[nH]ccc3c2/C=C/C(=O)OCc2ccccc2)ccc1F] | benzyl (E)-3-(5-(3- carbamimidoyl-4- fluorophenoxy)-6-fluoro- 1H-indol-4-yl)acrylate | 448 |
| 5-2 | 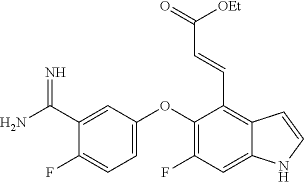 [SMILES: CCOC(=O)/C=C/c1c(Oc2ccc(F)c(C(=N)N)c2)c(F)cc2[nH]ccc12] | ethyl (E)-3-(5-(3- carbamimidoyl-4- fluorophenoxy)-6-fluoro- 1H-indol-4-yl)acrylate | 386 |
| 5-3 | 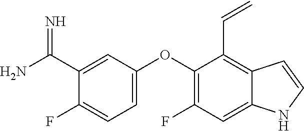 [SMILES: C=Cc1c(Oc2ccc(F)c(C(=N)N)c2)c(F)cc2[nH]ccc12] | 2-fluoro-5-((6-fluoro-4- vinyl-1H-indol-5- yl)oxy)benzimidamide | 314 |
| 5-4 | 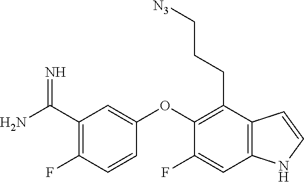 [SMILES: [N-]=[N+]=NCCCc1c(Oc2ccc(F)c(C(=N)N)c2)c(F)cc2[nH]ccc12] | 5-((4-(3-azidopropyl)-6- fluoro-1H-indol-5- yl)oxy)-2- fluorobenzimidamide | 371 |
| 5-5 | 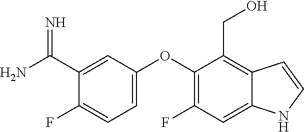 [SMILES: N=C(N)c1cc(Oc2c(F)cc3[nH]ccc3c2CO)ccc1F] | 2-fluoro-5-((6-fluoro-4- (hydroxymethyl)-1H- indol-5- yl)oxy)benzimidamide | 318 |
| 5-6 | 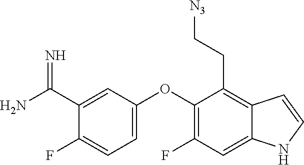 [SMILES: [N-]=[N+]=NCCc1c(Oc2ccc(F)c(C(=N)N)c2)c(F)cc2[nH]ccc12] | 5-((4-(2-Azidoethyl)-6- fluoro-1H-indol-5- yl)oxy)-2- fluorobenzimidamide | 357 |
| 5-7 | 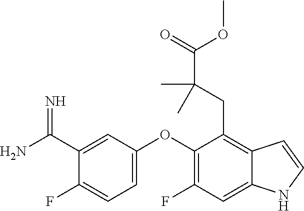 [SMILES: COC(=O)C(C)(C)Cc1c(Oc2ccc(F)c(C(=N)N)c2)c(F)cc2[nH]ccc12] | methyl 3-(5-(3- carbamimidoyl-4- fluorophenoxy)-6-fluoro- 1H-indol-4-yl)-2,2- dimethylpropanoate | 402 |
| 5-8 | 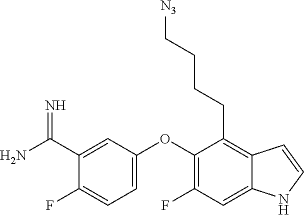 [SMILES: [N-]=[N+]=NCCCCc1c(Oc2ccc(F)c(C(=N)N)c2)c(F)cc2[nH]ccc12] | 5-((4-(4-azidobutyl)-6- fluoro-1H-indol-5- yl)oxy)-2- fluorobenzimidamide | 385 |
| 5-9 | 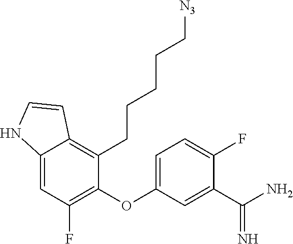 [SMILES: [N-]=[N+]=NCCCCCc1c(Oc2ccc(F)c(C(=N)N)c2)c(F)cc2[nH]ccc12] | 5-((4-(5-azidopentyl)-6- fluoro-1H-indol-5- yl)oxy)-2- fluorobenzimidamide | 399 |
| 5-10 | 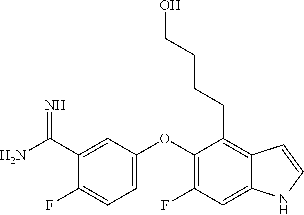 [SMILES: N=C(N)c1cc(Oc2c(F)cc3[nH]ccc3c2CCCCO)ccc1F] | 2-fluoro-5-((6-fluoro-4- (4-hydroxybutyl)-1H- indol-5- yl)oxy)benzimidamide | 360 |
| 5-11 | 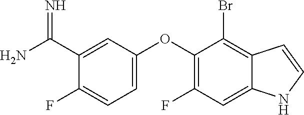 [SMILES: N=C(N)c1cc(Oc2c(F)cc3[nH]ccc3c2Br)ccc1F] | 5-((4-bromo-6-fluoro-1H- indol-5-yl)oxy)-2- fluorobenzimidamide | 366, 368 |
| 5-12 | 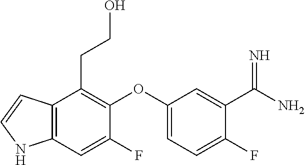 [SMILES: N=C(N)c1cc(Oc2c(F)cc3[nH]ccc3c2CCO)ccc1F] | 2-fluoro-5-((6-fluoro-4- (2-hydroxyethyl)-1H- indol-5- yl)oxy)benzimidamide | 332 |
| 5-13 | 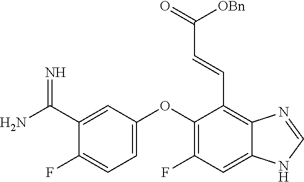 [SMILES: N=C(N)c1cc(Oc2c(F)cc3[nH]cnc3c2/C=C/C(=O)OCc2ccccc2)ccc1F] | benzyl (E)-3-(5-(3- carbamimidoyl-4- fluorophenoxy)-6-fluoro- 1H-benzo[d]imidazol-4- yl)acrylate | 449 |
| 5-14 | 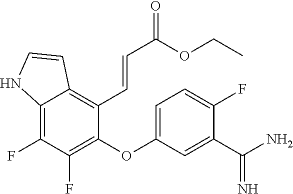 [SMILES: CCOC(=O)/C=C/c1c(Oc2ccc(F)c(C(=N)N)c2)c(F)c(F)c2[nH]ccc12] | ethyl (E)-3-(5-(3- carbamimidoyl-4- fluorophenoxy)-6,7- difluoro-1H-indol-4- yl)acrylate | 404 |

### Intermediate 6: Ethyl 2-(5-(3-carbamimidoyl-4-fluorophenoxy)-6-fluoro-1H-indol-4-yl)acetate

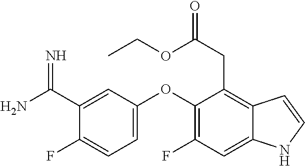 [SMILES: CCOC(=O)Cc1c(Oc2ccc(F)c(C(=N)N)c2)c(F)cc2[nH]ccc12]

To a stirred solution of ethyl 2-(5-(3-(N-acetoxycarbamimidoyl)-4-fluorophenoxy)-6-fluoro-1H-indol-4-yl)acetate (Intermediate 6C, 2.066 g, 4.8 mmol) in acetic acid (11 mL) was added palladium on carbon (10%, 50% wet, 214 mg). The reaction was stirred at room temperature overnight under hydrogen balloon. The pH was adjusted to ˜8 with saturated sodium bicarbonate. The mixture was diluted with ethyl acetate (300 mL), washed with water, brine, dried over sodium sulfate, and concentrated. The residue was purified by automated silica gel column chromatography (40 g column, eluting with 0-10% methanol in dichloromethane) to give the title compound (1.37 g, 76%) as green-yellow solid. MS (ESI): 374 m/z [M+H]⁺, retention time: 1.57 minutes, purity: 78% (254 nm) (LC-MS method 2).

The following intermediates were prepared based on the procedures described for Intermediate 6.

| Inter. | | | |
|---|---|---|---|
| No. | Structure | Name | MS m/z [M + H]⁺ and/or ¹HNMR |
| 6-1 | 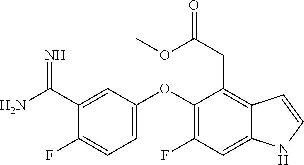 [SMILES: COC(=O)Cc1c(Oc2ccc(F)c(C(=N)N)c2)c(F)cc2[nH]ccc12] | methyl 2-(5-(3- carbamimidoyl-4- fluorophenoxy)-6-fluoro- 1H-indol-4-yl)acetate | 360 |
| 6-2 | 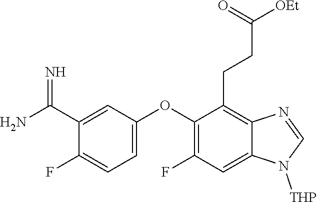 | ethyl 3-(5-(3- carbamimidoyl-4- fluorophenoxy)-6-fluoro-1- (tetrahydro-2H-pyran-2-yl)- 1H-benzo[d]imidazol-4- yl)propanoate | 473 |

### Intermediate 6A: Ethyl 2-(5-(3-cyano-4-fluorophenoxy)-6-fluoro-1-tosyl-1H-indol-4-yl)acetate

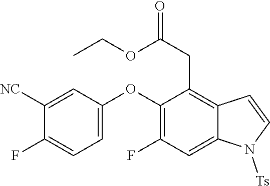 [SMILES: CCOC(=O)Cc1c(Oc2ccc(F)c(C#N)c2)c(F)cc2c1ccn2S(=O)(=O)c1ccc(C)cc1]

In a glovebox, to a stirred solution of 5-((4-bromo-6-fluoro-1-tosyl-1H-indol-5-yl)oxy)-2-fluorobenzonitrile (Intermediate 3, 8 g, 16 mmol) in N,N-dimethylformamide (65 mL) was added ethyl 2-tributylstannylacetate (15.1 g, 40 mmol), dichlorobis(tri-o-tolylphosphine)palladium(II) (1.261 g, 1.6 mmol) and zinc bromide (7.2 g, 32 mmol). The reaction was stirred at 100° C. under nitrogen overnight, cooled to room temperature and diluted with ethyl acetate (800 mL). The mixture was washed with water, brine, dried over sodium sulfate, and concentrated. The residue was purified by automated silica gel column chromatography (80 g column, eluting with 0-50% ethyl acetate in petroleum ether) to give the title compound (4.6 g, 57%) as a white solid. MS (ESI): 533 m/z [M+Na]⁺, retention time: 2.20 minutes, purity: 95% (214 nm) (LC-MS method 2).

The following intermediate was prepared based on the procedure described for Intermediate 6A.

| Inter. | | | MS m/z [M + H]⁺ and/or |
|---|---|---|---|
| No. | Structure | Name | ¹H NMR |
| 6A-1 | 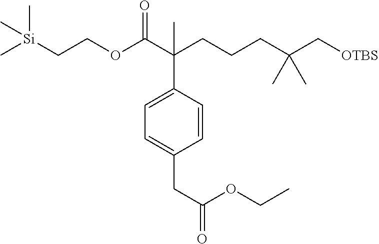 [SMILES: CCOC(=O)Cc1ccc(C(C)(CCCC(C)(C)CO[Si](C)(C)C(C)(C)C)C(=O)OCC[Si](C)(C)C)cc1] | 2-(trimethylsilyl)ethyl 7-((tert- butyldimethylsilyl)oxy)-2-(4-(2- ethoxy-2-oxoethyl)phenyl)-2,6,6- trimethylheptanoate | 565 |

### Intermediate 6B: Ethyl 2-(5-(3-cyano-4-fluorophenoxy)-6-fluoro-1H-indol-4-yl)acetate

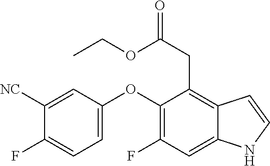 [SMILES: CCOC(=O)Cc1c(Oc2ccc(F)c(C#N)c2)c(F)cc2[nH]ccc12]

To a stirred solution of ethyl 2-(5-(3-cyano-4-fluorophenoxy)-6-fluoro-1-tosyl-1H-indol-4-yl)acetate (Intermediate 6A, 1.93 g, 3.8 mmol) in tetrahydrofuran (15.9 mL) was added tetrabutyl-ammonium fluoride (22.7 mL, 22.7 mmol). The reaction was stirred at 75° C. overnight, cooled to room temperature, and diluted with ethyl acetate (500 mL). The mixture was washed with water, brine, dried over sodium sulfate, and concentrated. The residue was purified by automated silica gel column chromatography (40 g silica gel column, eluting with 0-100% ethyl acetate in petroleum ether) to give the title compound (1.027 g, 76%) as a yellow solid. MS (ESI): 357 m/z [M+H]⁺, retention time: 2.09 minutes, purity: 87% (214 nm) (LC-MS method 5).

The following intermediate was prepared based on the procedures described for Intermediate 6B.

| Inter. | | | MS m/z [M + H]⁺ and/or |
|---|---|---|---|
| No. | Structure | Name | ¹H NMR |
| 6B-1 | 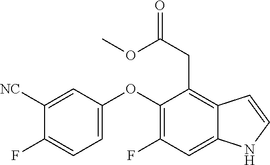 [SMILES: COC(=O)Cc1c(Oc2ccc(F)c(C#N)c2)c(F)cc2[nH]ccc12] | methyl 2-(5-(3-cyano-4- fluorophenoxy)-6-fluoro- 1H-indol-4-yl)acetate | 343 |

### Intermediate 6C: Ethyl 2-(5-(3-(N-acetoxycarbamimidoyl)-4-fluorophenoxy)-6-fluoro-1H-indol-4-yl)acetate

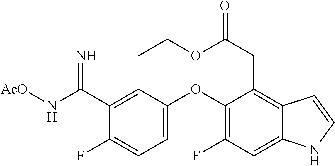 [SMILES: CCOC(=O)Cc1c(Oc2ccc(F)c(C(=N)NOC(C)=O)c2)c(F)cc2[nH]ccc12]

To a stirred solution of ethyl 2-(5-(3-cyano-4-fluorophenoxy)-6-fluoro-1H-indol-4-yl)acetate (Intermediate 6B, 2.063 g, 5.8 mmol) in dry methanol (22 mL) was added hydroxylamine hydrochloride (1.2 g, 17.4 mmol) and triethylamine (2.4 mL, 17.4 mmol). The reaction was stirred at room temperature overnight, then diluted with ethyl acetate (500 mL). The mixture was washed with water, brine, dried over sodium sulfate, and concentrated. The crude product, ethyl 2-(6-fluoro-5-(4-fluoro-3-(N-hydroxycarbamimidoyl)phenoxy)-1H-indol-4-yl)acetate, was used for the next step without further purification. MS (ESI): 390 m/z [M+H]⁺, retention time: 1.63 minutes, purity: 92% (254 nm) (LC-MS method 5).

To a stirred and cooled (0° C.) solution of the above crude product (2.159 g, 5.55 mmol) in acetic acid (21 mL) was added acetic anhydride (0.79 mL, 8.33 mmol). The reaction was stirred at room temperature overnight, then diluted with ethyl acetate (300 mL). The mixture was washed with brine, dried over sodium sulfate, and concentrated. The residue was purified by silica gel column chromatography (40 g column, eluting with 0-100% ethyl acetate in petroleum ether) to give the title compound (2.066 g, 86% two steps) as a yellow solid. MS (ESI): 432 m/z [M+H]⁺, retention time: 1.83 minutes, purity: 97% (254 nm) (LC-MS method 2).

The following intermediate was prepared based on the procedures described for Intermediate 6C.

| Inter. | | | MS m/z [M + H]⁺ and/or |
|---|---|---|---|
| No. | Structure | Name | ¹H NMR |
| 6C-1 | 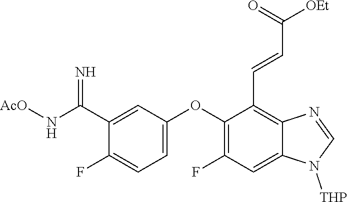 | ethyl (E)-3-(5-(3-(N- acetoxycarbamimidoyl)-4- fluorophenoxy)-6-fluoro-1- (tetrahydro-2H-pyran-2-yl)- 1H-benzo[d]imidazol-4- yl)acrylate | 529 |

### Intermediate 7: Methyl 2-(5-(3-cyano-4-fluorophenoxy)-6-fluoro-1-tosyl-1H-indol-4-yl)acetate

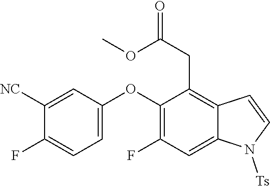 [SMILES: COC(=O)Cc1c(Oc2ccc(F)c(C#N)c2)c(F)cc2c1ccn2S(=O)(=O)c1ccc(C)cc1]

To a stirred solution of 2-(5-(3-cyano-4-fluorophenoxy)-6-fluoro-1-tosyl-1H-indol-4-yl)acetic acid (Intermediate 7C, 1 g, 2.07 mmol) in methanol (30 mL) was added concentrated sulfuric acid (1 mL). The reaction was stirred at room temperature overnight and concentrated. The residue was dissolved in ethyl acetate (50 mL). The solution was washed with water, saturated sodium bicarbonate, brine, dried over sodium sulfate, and concentrated. The crude title compound (1 g, 97%) was obtained as a solid. MS (ESI): 497 m/z [M+H]⁺, retention time: 2.19 minutes, purity: 87% (254 nm) (LC-MS method 7).

### Intermediate 7A: (E)-5-((4-(2-Ethoxyvinyl)-6-fluoro-1-tosyl-1H-indol-5-yl)oxy)-2-fluorobenzonitrile

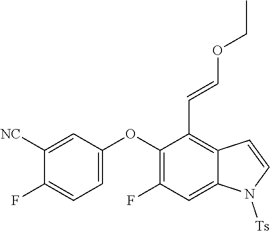 [SMILES: CCO/C=C/c1c(Oc2ccc(F)c(C#N)c2)c(F)cc2c1ccn2S(=O)(=O)c1ccc(C)cc1]

To a stirred and degassed solution of 5-((4-bromo-6-fluoro-1-tosyl-1H-indol-5-yl)oxy)-2-fluorobenzonitrile (Intermediate 3, 5.0 g, 19.93 mmol)) in dimethylformamide (50 mL) was added tributyl(2-ethoxyvinyl)stannane (4.3 g, 11.92 mmol), bis(triphenylphosphine) palladium(II) dichloride (695 mg, 0.99 mmol) and lithium chloride (1.3 g, 29.79 mmol) in a sealed tube. The mixture was heated at 100° C. for 4 hours, cooled to room temperature, and quenched with saturated potassium fluoride (50 mL). The solution was extracted with ethyl acetate (3×50 mL). The combined organic phases were washed with water, brine, dried over sodium sulfate, and concentrated. The residue was purified by silica gel column chromatography (petroleum ether/ethyl acetate=4:1) to give the title compound (3.4 g, 66%) as yellow solid. MS: 495 m/z [M+H]⁺.

The following intermediate was prepared based on the procedure described for Intermediate 7A.

| Inter. | | | MS m/z [M + H]⁺ and/or |
|---|---|---|---|
| No. | Structure | Name | ¹H NMR |
| 7A-1 | 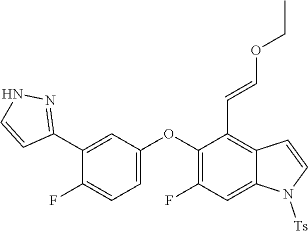 [SMILES: CCO/C=C/c1c(Oc2ccc(F)c(-c3cc[nH]n3)c2)c(F)cc2c1ccn2S(=O)(=O)c1ccc(C)cc1] | (E)-4-(2-ethoxyvinyl)-6-fluoro- 5-(4-fluoro-3-(1H-pyrazol-3- yl)phenoxy)-1-tosyl-1H-indole | 536 |

### Intermediate 7B: 2-Fluoro-5-((6-fluoro-4-(2-oxoethyl)-1-tosyl-1H-indol-5-yl)oxy)benzonitrile

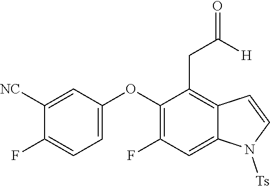 [SMILES: Cc1ccc(S(=O)(=O)n2ccc3c(CC=O)c(Oc4ccc(F)c(C#N)c4)c(F)cc32)cc1]

To a stirred solution of (E)-5-((4-(2-ethoxyvinyl)-6-fluoro-1-tosyl-1H-indol-5-yl)oxy)-2-fluorobenzonitrile (Intermediate 7A, 3.3 g, 6.67 mmol) in tetrahydrofuran (100 mL) was added 1 M hydrochloric acid (100 mL). The mixture was stirred at room temperature for 16 hours, then extracted with ethyl acetate (3×100 mL). The combined organic phases were washed with brine, dried over sodium sulfate, and concentrated to give the title compound (3.2 g, 100%) as a yellow oil. MS: 467 m/z [M+H]⁺.

### Intermediate 7C: 2-(5-(3-Cyano-4-fluorophenoxy)-6-fluoro-1-tosyl-1H-indol-4-yl)acetic acid

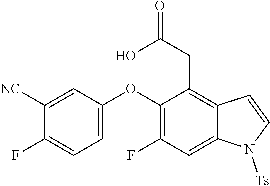 [SMILES: Cc1ccc(S(=O)(=O)n2ccc3c(CC(=O)O)c(Oc4ccc(F)c(C#N)c4)c(F)cc32)cc1]

To a stirred solution of 2-fluoro-5-((6-fluoro-4-(2-oxoethyl)-1-tosyl-1H-indol-5-yl)oxy)benzonitrile (Intermediate 7B, 1 g, 2.15 mmol) and 2-methyl-2-butene (3.2 mL, 30.1 mmol) in tetrahydrofuran (30 mL) and t-butanol (30 mL) was added a solution of sodium phosphate monobasic (1.29 g, 10.75 mmol) and sodium chlorite (774 mg, 8.6 mmol) in water (10 mL). The mixture was stirred at room temperature for 1 hour, diluted with 1N hydrochloric acid (100 mL), and extracted with ethyl acetate (3×40 mL). The combined organic extracts were washed with brine, dried over sodium sulfate, and concentrated to give the title compound (1 g, 96%) as a yellow solid. MS (ESI): 483 m/z [M+H]⁺, retention time: 2.05 minutes, purity: 90% (254 nm) (LC-MS method 3).

### Intermediate 8: (E)-6-Cyano-2-(3-(3-methoxy-2-methyl-3-oxoprop-1-en-1-yl)phenyl)hexanoic acid

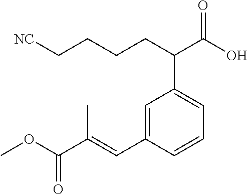 [SMILES: COC(=O)/C(C)=C/c1cccc(C(CCCCC#N)C(=O)O)c1]

To a stirred and degassed solution of 2-(3-bromophenyl)-6-cyanohexanoic acid (Intermediate 1A-1, 1.2 g, 4.0 mmol) in 20 mL of N,N-dimethylformamide was added methyl methacrylate (1.2 g, 12 mmol), tri (o-tolyl)phosphine (365 mg, 1.2 mmol), triethylamine (1.2 g, 12 mmol) and palladium (II) acetate (45 mg, 0.4 mmol). The mixture was stirred at 110° C. for 2 hours, cooled to room temperature, and quenched with water (50 mL). The solution was extracted with ethyl acetate (3×20 mL). The combined organic phases were dried over magnesium sulfate and concentrated. The residue was purified by silica gel column chromatography (eluting with ethyl acetate/petroleum ether (50:50)) to provide the title compound (600 mg, 60%) as a white solid. MS (ESI): 316 m/z [M+H]⁺, retention time: 1.22 minutes, purity: 80% (214 nm) (LC-MS Method 2).

### Intermediate 9: 2-Fluoro-5-((6-fluoro-1-tosyl-4-vinyl-1H-indol-5-yl)oxy)benzonitrile

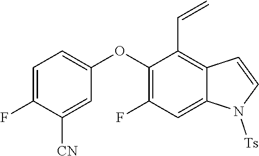 [SMILES: C=Cc1c(Oc2ccc(F)c(C#N)c2)c(F)cc2c1ccn2S(=O)(=O)c1ccc(C)cc1]

To a suspension of 5-((4-bromo-6-fluoro-1-tosyl-1H-indol-5-yl)oxy)-2-fluorobenzonitrile (Intermediate 3, 5.25 g, 10.5 mmol) in dioxane (120 mL) and water (40 mL) were added 4,4,5,5-tetramethyl-2-vinyl-1,3,2-dioxaborolane (3.2 g, 21 mmol), Pd(dppf)Cl₂ (384 mg, 0.5 mmol) and cesium carbonate (6.8 g, 21 mmol). The reaction mixture was stirred at 100° C. overnight, cooled to room temperature and extracted with ethyl acetate (150 mL×3). The combined organic extracts were washed with brine, dried over magnesium sulfate, and evaporated to dryness. The resulting residue was purified by flash chromatography over silica (petroleum ether/dichloromethane, v/v, 2/1) to afford the title compound as a yellow solid (4 g, 85%). MS: 473 m/z [M+Na]⁺.

The following intermediates were prepared utilizing the procedure described for Intermediate 9.

| Inter. | | | MS m/z |
|---|---|---|---|
| No. | Structure | Name | [M + H]⁺ |
| 9-1 | 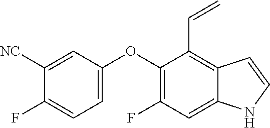 [SMILES: C=Cc1c(Oc2ccc(F)c(C#N)c2)c(F)cc2[nH]ccc12] | 2-fluoro-5-((6-fluoro-4-vinyl-1H-indol- 5-yl)oxy)benzonitrile | 297 |
| 9-2 | 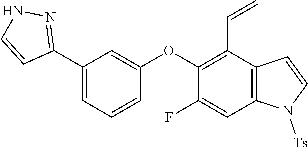 [SMILES: C=Cc1c(Oc2cccc(-c3cc[nH]n3)c2)c(F)cc2c1ccn2S(=O)(=O)c1ccc(C)cc1] | 5-(3-(1H-pyrazol-3-yl)phenoxy)-6- fluoro-1-tosyl-4-vinyl-1H-indole | 474 |
| 9-3 | 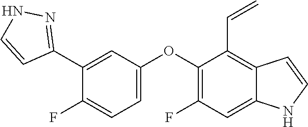 [SMILES: C=Cc1c(Oc2ccc(F)c(-c3cc[nH]n3)c2)c(F)cc2[nH]ccc12] | 6-fluoro-5-(4-fluoro-3-(1H-pyrazol-3- yl)phenoxy)-4-vinyl-1H-indole | 338 |

### Intermediate 10: 3-(3-(((Benzyloxy)carbonyl)(methyl)amino)propoxy)-2-(3-iodophenyl)-2-methylpropanoic acid

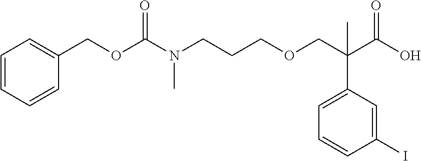 [SMILES: CN(CCCOCC(C)(C(=O)O)c1cccc(I)c1)C(=O)OCc1ccccc1]

To a stirred solution of methyl 3-(3-(((benzyloxy)carbonyl)(methyl)amino)propoxy)-2-(3-iodophenyl)-2-methylpropanoate (Intermediate 10C, 250 mg, 0.47 mmol) in tetrahydrofuran (3 mL) and water (1 mL) was added sodium hydroxide (57 mg, 1.43 mmol). The mixture was heated to 50° C. and stirred for 16 hours, cooled to room temperature, and quenched with saturate ammonium chloride solution (20 mL). The mixture was extracted with ethyl acetate (3×10 mL). The combined organic phases were washed with brine (20 mL), dried over sodium sulfate, and concentrated. The residue was purified by silica gel column chromatography (eluting with dichloromethane/methanol (95:5)) to afford the title compound (200 mg, 69% for two steps) as a solid. MS (ESI): 512 m/z [M+H]⁺, retention time: 1.46 minutes, purity: 95% (214 nm) (LC-MS method 9).

The following intermediates were prepared utilizing the procedures described for Intermediate 10.

| Inter. | | | MS m/z |
|---|---|---|---|
| No. | Structure | Name | [M + H]⁺ |
| 10-1 | 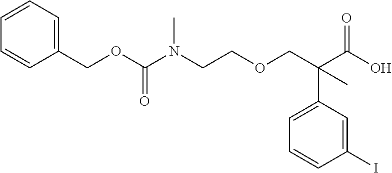 [SMILES: CN(CCOCC(C)(C(=O)O)c1cccc(I)c1)C(=O)OCc1ccccc1] | 3-(2- (((benzyloxy)carbonyl)(methyl)amino) ethoxy)-2-(3-iodophenyl)-2- methylpropanoic acid | 498 |
| 10-2 | 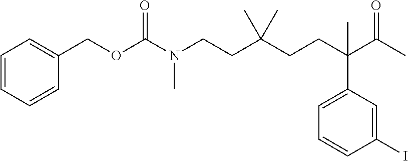 [SMILES: CC(=O)C(C)(CCC(C)(C)CCN(C)C(=O)OCc1ccccc1)c1cccc(I)c1] | 7- (((benzyloxy)carbonyl)(methyl)amino)- 2-(3-iodophenyl)-2,5,5- trimethylheptanoic acid | 538 |

### Intermediate 10A: 1-Bromo-3-(chloromethoxy)propane

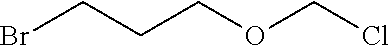

Through a stirred and cooled (0° C.) solution of 3-bromo-1-propanol (16 g., 116 mmol) and paraformaldehyde (4.5 g, 150 mmol) in dichloromethane (20 mL) was passed a slow stream of dry hydrogen chloride (g) for 3 hours. The separated organic phase was dried over calcium chloride and concentrated to give the title compound (18 g, 83%) as an oil. ¹H-NMR (CDCl₃, 400 MHz) δ 5.51 (s, 2H), 3.85 (t, J=7.6 Hz, 2H), 3.49 (t, J=7.2 Hz, 2H), 2.10-2.18 (m, 2H) ppm.

The following intermediate was prepared utilizing the procedures described for Intermediate 10A.

| Inter. | | | MS m/z |
|---|---|---|---|
| No. | Structure | Name | [M + H]⁺ |
| 10A-1 | 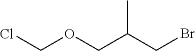 | 1-bromo-3- (chloromethoxy)-2- methylpropane | ¹H NMR (400 MHz, CDCl₃) δ 5.49 (s, 2H), 3.60-3.70 (m, 2H), 3.45 (d, J = 5.2 Hz, 2H), 2.11-2.19 (m, 1H), 1.06 (d, J = 6.8 Hz, 3H) ppm |

### Intermediate 1OB: Methyl 3-(3-bromopropoxy)-2-(3-iodophenyl)-2-methylpropanoate

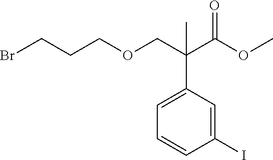 [SMILES: COC(=O)C(C)(COCCCBr)c1cccc(I)c1]

To a stirred and cooled (−78° C.) solution of methyl 2-(3-iodophenyl)propanoate (191 mg, 0.66 mmol) in tetrahydrofuran (5 mL) was added lithium bis(trimethylsilyl)amide (0.5 mL in tetrahydrofuran, 1 mmol). The mixture was stirred at −78° C. for 0.5 hours, then treated with 1-bromo-3-(chloromethoxy)propane (Intermediate 10A, 186 mg, 1 mmol) in tetrahydrofuran (1 mL) and stirred at −78° C. for another 1 hour. The reaction was quenched with saturated ammonium chloride (20 mL), warmed to room temperature, and extracted with ethyl acetate (3×20 mL). The combined organic phases were washed with brine (20 mL), dried over sodium sulfate, and concentrated. The residue was purified by silica gel column chromatography (eluting with ethyl acetate/petroleum ether (20:80)) to afford the title compound (250 mg, 55%) as an oil. MS (ESI): 441, 443 m/z [M+H]⁺, retention time: 1.98 minutes, purity: 95% (214 nm) (LC-MS method 9).

The following intermediates were prepared utilizing the procedures described for Intermediate 1 GB.

| Inter. | | | |
|---|---|---|---|
| No. | Structure | Name | MS m/z [M + H]⁺ |
| 10B-1 | 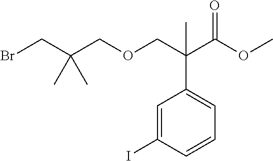 [SMILES: COC(=O)C(C)(COCC(C)(C)CBr)c1cccc(I)c1] | methyl 3-(3-bromo- 2,2- dimethylpropoxy)- 2-(3-iodophenyl)-2- methylpropanoate | 469, 471 |
| 10B-2 | 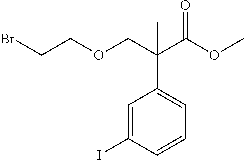 [SMILES: COC(=O)C(C)(COCCBr)c1cccc(I)c1] | methyl 3-(2- bromoethoxy)-2-(3- iodophenyl)-2- methylpropanoate | 427, 429 |
| 10B-3 | 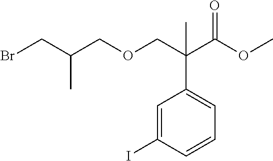 [SMILES: COC(=O)C(C)(COCC(C)CBr)c1cccc(I)c1] | methyl 3-(3-bromo- 2-methylpropoxy)- 2-(3-iodophenyl)-2- methylpropanoate | MS-ESI: 477, 479 m/z [M + Na]⁺. ¹HNMR (400MHz, CDCl₃) δ 7.63 (s, 1H), 7.60 (d, J = 7.6 Hz, 1H), 7.28-7.26 (m, 1H), 7.06- 7.04 (m, 1H), 3.94-3.89 (m, 1H), 3.69 (s, 3H), 3.67-3.61 (m, 1H), 3.45-3.31 (m, 4H), 2.07-2.04 (m, 1H), 1.62-1.59 (m, 3H), 0.99-0.96 (m, 3H) ppm |
| 10B-4 | 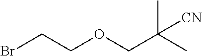 | 3-(2-bromoethoxy)- 2,2- dimethylpro- panenitrile | 206, 208 |
| 10B-5 | 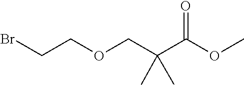 | methyl 3-(2- bromoethoxy)-2,2- dimethylpropanoate | 239, 241 |

### Intermediate 10C: Methyl 3-(3-(((benzyloxy)carbonyl)(methyl)amino)propoxy)-2-(3-iodophenyl)-2-methylpropanoate

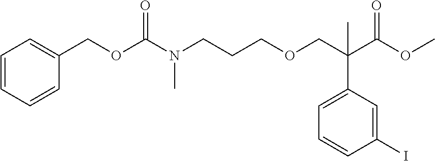 [SMILES: COC(=O)C(C)(COCCCN(C)C(=O)OCc1ccccc1)c1cccc(I)c1]

To a stirred and cooled (0° C.) solution of methyl 3-(3-bromopropoxy)-2-(3-iodophenyl)-2-methylpropanoate (Intermediate 10B, 140 mg, 0.85 mmol) and benzyl methylcarbamate (250 mg, 0.57 mmol) in N,N-dimethylformamide (1 mL) was added sodium hydride (35 mg, 0.85 mmol) in N,N-dimethylformamide (2 mL). The reaction mixture was stirred at room temperature for 4 hours, then quenched with water (20 mL) and extracted with ethyl acetate (3×10 mL). The combined organic phases were washed with brine, dried over sodium sulfate, and concentrated to give the crude mixture of title compound and its corresponding acid, 3-(3-(((benzyloxy)carbonyl)(methyl)amino)propoxy)-2-(3-iodophenyl)-2-methylpropanoic acid (Intermediate 10) (250 mg). This mixture was used for next step without further purification. MS (ESI): 526 m/z [M+H]⁺, purity: 33% (214 nm), and corresponding acid MS (ESI): 512 m/z [M+H]⁺, purity: 30% (214 nm) (LC-MS method 9).

The following intermediates were prepared utilizing the procedures described for Intermediate 10C.

| Inter. | | | |
|---|---|---|---|
| No. | Structure | Name | MS m/z [M + H]⁺ |
| 10C-1 | 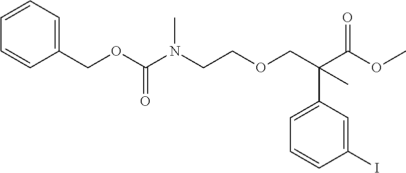 [SMILES: COC(=O)C(C)(COCCN(C)C(=O)OCc1ccccc1)c1cccc(I)c1] | methyl 3-(2- (((benzyloxy)carbonyl)(methyl)amino) ethoxy)-2-(3-iodophenyl)-2- methylpropanoate | 512 |
| 10C-2 | 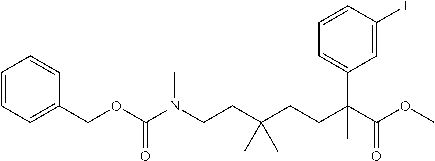 [SMILES: COC(=O)C(C)(CCC(C)(C)CCN(C)C(=O)OCc1ccccc1)c1cccc(I)c1] | methyl 7- (((benzyloxy)carbonyl)(methyl)amino)- 2-(3-iodophenyl)-2,5,5- trimethylheptanoate | 574 [M + Na]⁺ |

### Intermediate 11: Benzyl (3-(4-bromo-2-(3-iodophenyl)-2-methyl-3-oxobutoxy)propyl)(methyl)carbamate

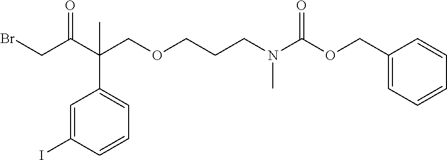 [SMILES: CN(CCCOCC(C)(C(=O)CBr)c1cccc(I)c1)C(=O)OCc1ccccc1]

To a stirred and cooled (0° C.) solution of benzyl (3-(2-(3-iodophenyl)-2-methyl-3-oxobutoxy)propyl)(methyl)carbamate (Intermediate 11B, 2.2 g, 4 mmol) in tetrahydrofuran (30 mL) was added pyridine hydrobromide perbromide (1.4 g, 4.4 mmol). The mixture was stirred at room temperature for 2 hours, then quenched with water and extracted with ethyl acetate (3×50 mL). The combined organic phase was washed with brine (50 mL), dried over sodium sulfate, and concentrated. The residue was purified by silica gel column chromatography eluting with ethyl acetate/petroleum ether (20:80) to afford the title compound as an oil (2 g, 80%). MS (ESI): 588, 590 m/z [M+H]⁺, retention time: 2.22 minutes, purity: 90% (214 nm) (LC-MS method 9).

The following intermediates were prepared utilizing the procedures described for Intermediate 11.

| Inter. | | | |
|---|---|---|---|
| No. | Structure | Name | MS m/z [M + H]⁺ |
| 11-1 | 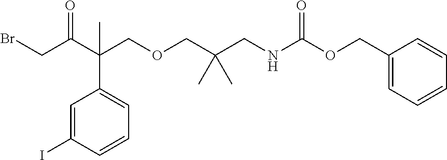 [SMILES: CC(C)(CNC(=O)OCc1ccccc1)COCC(C)(C(=O)CBr)c1cccc(I)c1] | benzyl (3-(4-bromo-2-(3-iodophenyl)- 2-methyl-3-oxobutoxy)-2,2- dimethylpropyl)carbamate | 602, 604 |
| 11-2 | 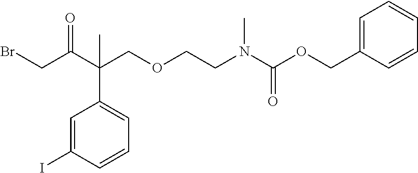 [SMILES: CN(CCOCC(C)(C(=O)CBr)c1cccc(I)c1)C(=O)OCc1ccccc1] | benzyl (2-(4-bromo-2-(3-iodophenyl)- 2-methyl-3- oxobutoxy)ethyl)(methyl)carbamate | 574, 576 |
| 11-3 | 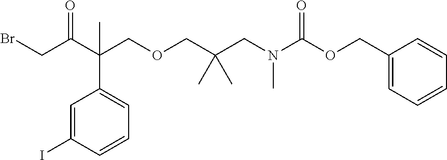 [SMILES: CN(CC(C)(C)COCC(C)(C(=O)CBr)c1cccc(I)c1)C(=O)OCc1ccccc1] | benzyl (3-(4-bromo-2-(3-iodophenyl)- 2-methyl-3-oxobutoxy)-2,2- dimethylpropyl)(methyl)carbamate | 616, 618 |
| 11-4 | 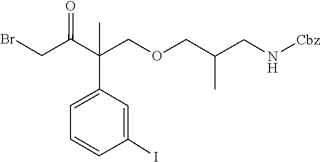 [SMILES: CC(CNC(=O)OCc1ccccc1)COCC(C)(C(=O)CBr)c1cccc(I)c1] | benzyl (3-(4-bromo-2-(3-iodophenyl)- 2-methyl-3-oxobutoxy)-2- methylpropyl)carbamate | 588, 590 |
| 11-5 | 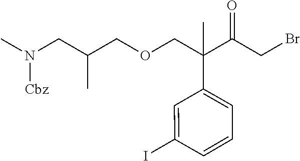 [SMILES: CC(COCC(C)(C(=O)CBr)c1cccc(I)c1)CN(C)C(=O)OCc1ccccc1] | benzyl (3-(4-bromo-2-(3-iodophenyl)- 2-methyl-3-oxobutoxy)-2- methylpropyl)(methyl)carbamate | 624, 626 [M + Na]⁺ |
| 11-6 | 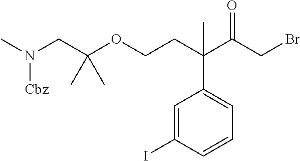 [SMILES: CN(CC(C)(C)OCCC(C)(C(=O)CBr)c1cccc(I)c1)C(=O)OCc1ccccc1] | benzyl (2-((5-bromo-3-(3-iodophenyl)- 3-methyl-4-oxopentyl)oxy)-2- methylpropyl)(methyl)carbamate | 638, 640 [M + Na]⁺ |
| 11-7 | 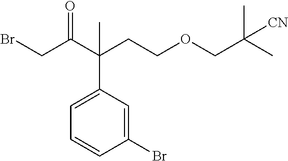 [SMILES: CC(C)(C#N)COCCC(C)(C(=O)CBr)c1cccc(Br)c1] | 3-((5-bromo-3-(3-bromophenyl)-3- methyl-4-oxopentyl)oxy)-2,2- dimethylpropanenitrile | 454 [M + Na]⁺ |
| 11-8 | 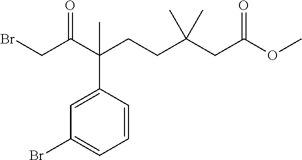 [SMILES: COC(=O)CC(C)(C)CCC(C)(C(=O)CBr)c1cccc(Br)c1] | methyl 8-bromo-6-(3-bromophenyl)- 3,3,6-trimethyl-7-oxooctanoate | 471 [M + Na]⁺ |

### Intermediate 11A: Benzyl (3-(2-(3-iodophenyl)-3-(methoxy(methyl)amino)-2-methyl-3-oxopropoxy)propyl)(methyl)carbamate

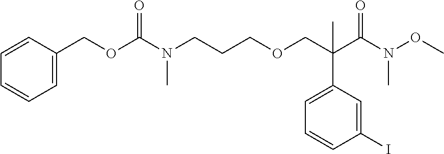 [SMILES: CON(C)C(=O)C(C)(COCCCN(C)C(=O)OCc1ccccc1)c1cccc(I)c1]

To a stirred solution of 3-(3-(((benzyloxy)carbonyl)(methyl)amino)propoxy)-2-(3-iodophenyl)-2-methylpropanoic acid (Intermediate 10, 2.5 g, 4.9 mmol) in N,N-dimethylformamide (40 mL) was added N,O-dimethylhydroxylamine (2.9 g, 29.4 mmol), triethylamine (3 g, 29.4 mmol) and 1-[bis(dimethylamino)methylene]-1h-1,2,3-triazolo[4,5-b]pyridinium 3-oxide hexafluorophosphate (HATU) (5.5 g, 14.6 mmol). The mixture was stirred for 16 hours at room temperature, quenched with water (200 mL) and extracted with ethyl acetate (3×100 mL). The combined organic phases were washed with brine, dried over sodium sulfate, and concentrated. The residue was purified by silica gel column chromatography (eluting with ethyl acetate/petroleum ether (30:70)) to afford the title compound (3.8 g, 70%) as a solid. MS (ESI): 555 m/z [M+H]⁺, retention time: 1.96 minutes, purity: 95% (214 nm) (LC-MS method 9).

The following intermediates were prepared utilizing the procedures described for Intermediate 11A.

| Inter. | | | |
|---|---|---|---|
| No. | Structure | Name | MS m/z [M + H]⁺ |
| 11A-1 | 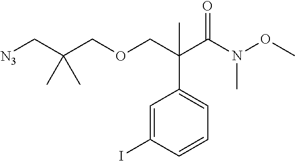 [SMILES: CON(C)C(=O)C(C)(COCC(C)(C)CN=[N+]=[N-])c1cccc(I)c1] | 3-(3-azido-2,2-dimethylpropoxy)-2-(3- iodophenyl)-N-methoxy-N,2- dimethylpropanamide | 461 |
| 11A-2 | 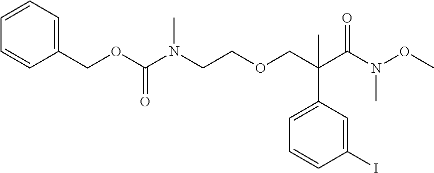 [SMILES: CON(C)C(=O)C(C)(COCCN(C)C(=O)OCc1ccccc1)c1cccc(I)c1] | benzyl (2-(2-(3-iodophenyl)-3- (methoxy(methyl)amino)-2-methyl-3- oxopropoxy)ethyl)(methyl)carbamate | 541 |
| 11A-3 | 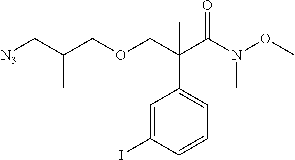 [SMILES: CON(C)C(=O)C(C)(COCC(C)CN=[N+]=[N-])c1cccc(I)c1] | 3-(3-azido-2-methylpropoxy)-2-(3- iodophenyl)-N-methoxy-N,2- dimethylpropanamide | 447 |
| 11A-4 | 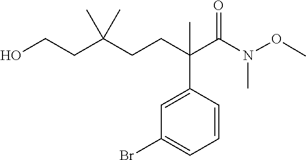 [SMILES: CON(C)C(=O)C(C)(CCC(C)(C)CCO)c1cccc(Br)c1] | 2-(3-bromophenyl)-7-hydroxy-N- methoxy-N,2,5,5- tetramethylheptanamide | 386, 388 |

### Intermediate 11B: Benzyl (3-(2-(3-iodophenyl)-2-methyl-3-oxobutoxy)propyl)(methyl)-carbamate

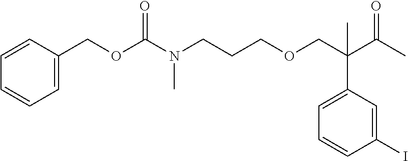 [SMILES: CC(=O)C(C)(COCCCN(C)C(=O)OCc1ccccc1)c1cccc(I)c1]

To a stirred and cooled (0° C.) solution of benzyl (3-(2-(3-iodophenyl)-3-(methoxy(methyl)amino)-2-methyl-3-oxopropoxy)propyl)(methyl)carbamate (Intermediate 11A, 3.8 g, 6.8 mmol) in tetrahydrofuran (40 mL) was added methyl magnesium bromide (34 mL, 2 M in tetrahydrofuran, 68 mmol). The mixture was stirred at room temperature for 16 hours, quenched with saturate ammonium chloride (300 mL) at 0° C., and extracted with ethyl acetate (3×100 mL). The combined organic phases were washed with brine, dried over sodium sulfate, and concentrated to give a mixture of the title compound and deprotected amine 3-(3-iodophenyl)-3-methyl-4-(3-(methylamino)propoxy)butan-2-one (4 g). This crude mixture was dissolved in tetrahydrofuran (40 mL), and treated with triethylamine (727 mg, 7.2 mmol) and benzyloxy carbonyl chloride (1.22 g, 7.2 mmol) at 0° C. The mixture was stirred at room temperature for 1 hour, quenched with water and extracted with ethyl acetate (3×50 mL). The combined organic phase was washed with brine (50 mL), dried over sodium sulfate, and concentrated. The residue was purified by silica gel column chromatography eluting with ethyl acetate/petroleum ether (20:80) to afford the title compound as an oil (2.2 g, 63% for 2 steps). MS (ESI): 510 m/z [M+H]⁺, retention time: 2.07 minutes, purity: 95% (214 nm) (LC-MS method 9).

### Intermediate 12: 5-((4-Allyl-6-fluoro-1H-indol-5-yl)oxy)-2-fluorobenzonitrile

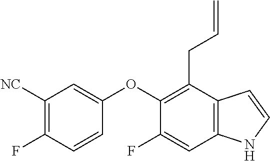 [SMILES: C=CCc1c(Oc2ccc(F)c(C#N)c2)c(F)cc2[nH]ccc12]

To a stirred and degassed solution of 5-((4-bromo-6-fluoro-1H-indol-5-yl)oxy)-2-fluorobenzonitrile (Intermediate 2, 10 g, 0.029 mol) in N,N′-dimethylformamide (100 mL) was added allyl(tributyl)stannane (11.4 g, 0.034 mol), lithium chloride (3.6 g, 0.086 mol), and bis-(triphenylphosphine)palladium(II) chloride (1.21 g, 1.72 mmol). The mixture was stirred at 90° C. under nitrogen overnight, then cooled to room temperature and treated with saturated potassium fluoride (300 mL). The solution was stirred for 10 minutes and filtered to remove the solid. The filtrate was extracted with ethyl acetate (3×200 mL). The combined organic extracts were washed with saturated lithium chloride solution, brine, dried over sodium sulfate, and concentrated. The residue was purified by flash column chromatography (ethyl acetate: petroleum ether=1:3) to give the title compound (9.5 g, 65%) as a light pink solid. MS (ESI): 311 m/z [M+H]⁺,

### Intermediate 13: 2-Fluoro-5-((6-fluoro-4-(3-hydroxypropyl)-1H-indol-5-yl)oxy)benzonitrile

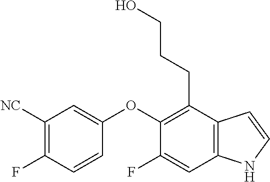 [SMILES: N#Cc1cc(Oc2c(F)cc3[nH]ccc3c2CCCO)ccc1F]

To a stirred and cooled (0° C.) solution of 5-((4-allyl-6-fluoro-1H-indol-5-yl)oxy)-2-fluoro-benzonitrile (intermediate 12, 3.7 g, 12 mmol) in dry tetrahydrofuran (60 ml) was added borane-dimethylsulfide complex (2 M in tetrahydrofuran, 12 mL, 24 mmol). The mixture was stirred at the same temperature for 2 hours, then treated with sodium acetate (6 N in water, 12 mL), followed by 30% hydrogen peroxide (5 mL), and stirred for additional 2 hours after the addition. The mixture was diluted with ethyl acetate (200 ml). The solution was washed with brine, dried over sodium sulfate, and concentrated. The residue was purified by automated flash chromatography (40 g silica gel column, eluting with 0-50% ethyl acetate in petroleum ether) to give the title compound (2.04 g, 52%) as a solid. MS (ESI): 329 m/z [M+H]⁺, retention time: 1.86 minutes, purity: 93% (214 nm) (LC-MS method 2). ¹H NMR (400 MHz, CDCl₃) δ 8.36 (s, 1H), 7.30-7.26 (m, 1H), 7.22-7.08 (m, 3H), 7.00 (dd, J=4.8, 3.1 Hz, 1H), 6.62 (dd, J=3.8, 1.5 Hz, 1H), 3.63 (d, J=3.4 Hz, 2H), 2.96-2.85 (m, 2H), 1.95-1.83 (m, 2H), 1.47 (s, 1H) ppm.

### Intermediate 14: 5-((4-(3-Azidopropyl)-6-fluoro-1H-indol-5-yl)oxy)-2-fluorobenzonitrile

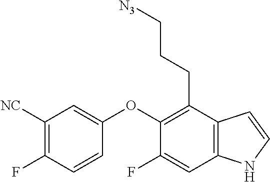 [SMILES: N#Cc1cc(Oc2c(F)cc3[nH]ccc3c2CCCN=[N+]=[N-])ccc1F]

To a solution of 3-(5-(3-cyano-4-fluorophenoxy)-6-fluoro-1H-indol-4-yl)propyl methanesulfonate (Intermediate 14A, 2.7 g, 6.65 mmol) in N,N-dimethylformamide (20 mL) was added sodium azide (0.865 g, 13.3 mmol). The mixture was stirred at 70° C. for 2 hours, cooled to room temperature and quenched with water (80 mL). The solution was extracted with ethyl acetate (3×30 mL). The combined organic extracts were washed with brine (3×20 mL), dried over sodium sulfate, and concentrated. The residue was purified by automated flash chromatography (40 g silica gel column, eluting with 0-50% ethyl acetate in petroleum ether) to give the title compound (1.68 g, 71%) as a solid. MS (ESI): 376 m/z [M+Na]⁺, retention time: 2.16 minutes, purity: >99% (214 nm) (LC-MS method 3). ¹H NMR (400 MHz, CDCl₃) δ 8.35 (s, 1H), 7.33-7.27 (m, 1H), 7.22-7.08 (m, 3H), 7.00 (dd, J=4.8, 3.1 Hz, 1H), 6.68-6.52 (m, 1H), 3.32-3.27 (m, 2H), 2.95-2.83 (m, 2H), 1.98-1.84 (m, 2H).

### Intermediate 14A: 3-(5-(3-Cyano-4-fluorophenoxy)-6-fluoro-1H-indol-4-yl)propyl methanesulfonate

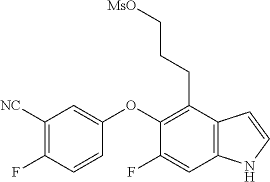 [SMILES: CS(=O)(=O)OCCCc1c(Oc2ccc(F)c(C#N)c2)c(F)cc2[nH]ccc12]

To a stirred and cooled (0° C.) solution of 2-fluoro-5-((6-fluoro-4-(3-hydroxypropyl)-1H-indol-5-yl)oxy)benzonitrile (Intermediate 13, 2.205 g, 6.72 mmol) in dichloromethane (30 mL) was added methanesulfonyl chloride (0.919 g, 8.06 mmol) and triethylamine (1.4 mL, 10.08 mmol). The mixture was stirred at 0° C. for 2 hours and diluted with dichloromethane (30 mL). The solution was washed with brine, dried over sodium sulfate, and concentrated to give the crude title compound (2.7 g, 99%) as a solid. MS (ESI): 407 m/z [M+H]⁺, retention time: 1.93 minutes, purity: 90% (214 nm) (LC-MS method 9).

### Intermediate 15: 7-Azido-2-(3-iodophenyl)heptanoic acid

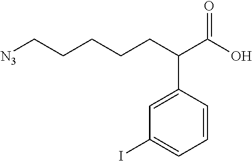 [SMILES: [N-]=[N+]=NCCCCCC(C(=O)O)c1cccc(I)c1]

To a solution of methyl 7-azido-2-(3-iodophenyl)heptanoate (Intermediate 15B, 700 mg, 1.8 mmol) in methanol (20 mL) and water (10 mL) was added potassium hydroxide (1 g, 18 mmol). The reaction was stirred at room temperature for 1.5 hours and concentrated to remove methanol. The residue was dissolved in water (100 mL) and extracted with ethyl acetate (30 mL). The organic layer was discarded. The aqueous phase was acidified to pH˜4 with concentrated hydrochloric acid and extracted with ethyl acetate (3×50 mL). The combined organic extracts were washed with brine, dried over sodium sulfate, and concentrated to give the title compound (628 mg, 89%) as a solid. MS (ESI): 396 m/z [M+Na]⁺, retention time: 2.09 minutes, purity: 98% (214 nm) (LC-MS method 7).

The following intermediate was prepared utilizing the procedures described for Intermediate 15.

| Inter. | | | |
|---|---|---|---|
| No. | Structure | Name | MS m/z [M + H]⁺ |
| 15-1 | 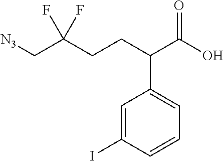 [SMILES: [N-]=[N+]=NCC(F)(F)CCC(C(=O)O)c1cccc(I)c1] | 6-azido-5,5-difluoro-2-(3- iodophenyl)hexanoic acid | 418 [M + Na]⁺ |

### Intermediate 15A: Methyl 7-chloro-2-(3-iodophenyl)heptanoate

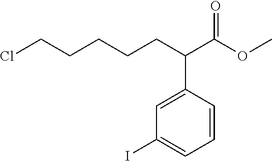 [SMILES: COC(=O)C(CCCCCCl)c1cccc(I)c1]

To a stirred solution of 7-chloro-2-(3-iodophenyl)heptanoic acid (Intermediate 1A-5, 1.1 g, 3.14 mmol) in methanol (10 mL) was added sulfuric acid (1 mL). The reaction mixture was refluxing overnight and concentrated. The residue was dissolved in ethyl acetate and washed water, brine, dried over sodium sulfate, and concentrated to give the title compound as a solid (1.1 g, 92%). MS (ESI): 381 m/z [M+H]⁺, retention time: 2.29 minutes, purity: 91% (254 nm) (LC-MS method 7).

The following intermediates were prepared utilizing the procedures described for Intermediate 15A.

| Inter. | | | |
|---|---|---|---|
| No. | Structure | Name | MS m/z [M + H]⁺ |
| 15A-1 | 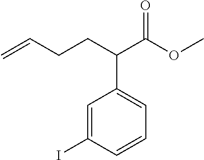 [SMILES: C=CCCC(C(=O)OC)c1cccc(I)c1] | methyl 2-(3-iodophenyl)hex-5-enoate | 331 |
| 15A-2 | 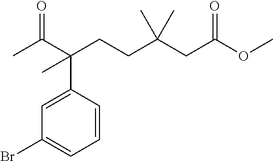 [SMILES: COC(=O)CC(C)(C)CCC(C)(C(C)=O)c1cccc(Br)c1] | methyl 6-(3-bromophenyl)-3,3,6-trimethyl- 7-oxooctanoate | 369, 371 |

### Intermediate 15B: Methyl 7-azido-2-(3-iodophenyl)heptanoate

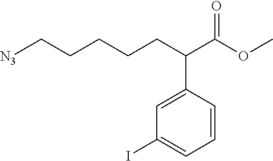 [SMILES: COC(=O)C(CCCCCN=[N+]=[N-])c1cccc(I)c1]

To a stirred solution of methyl 7-chloro-2-(3-iodophenyl)heptanoate (Intermediate 15A, 1 g, 2.6 mmol) in N,N-dimethylformamide (15 mL) was added sodium azide (1.02 g, 15.8 mmol) and potassium iodide (870 mg, 5.6 mmol). The reaction mixture was heated at 80° C. overnight and concentrated. The residue was dissolved in ethyl acetate (100 mL), washed with brine, dried over sodium sulfate, and concentrated. The crude product was purified by automated silica gel column chromatography (eluting with petroleum ether/ethyl acetate=0-30%) to give the title compound (0.701 g, 70%) as an oil. MS (ESI): 410 m/z [M+Na]⁺, retention time: 2.27 minutes, purity: 96% (214 nm) (LC-MS method 7).

### Intermediate 16: 2-Fluoro-5-((6-fluoro-4-formyl-1-tosyl-1H-indol-5-yl)oxy)benzonitrile

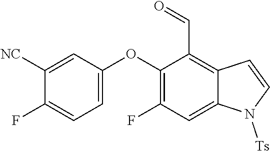 [SMILES: Cc1ccc(S(=O)(=O)n2ccc3c(C=O)c(Oc4ccc(F)c(C#N)c4)c(F)cc32)cc1]

To a stirred and cooled (0° C.) solution of 2-fluoro-5-((6-fluoro-1-tosyl-4-vinyl-1H-indol-5-yl)oxy)benzonitrile (Intermediate 9, 4.6 g, 10.2 mmol) in tetrahydrofuran (90 mL) were added 2,6-lutidine (1.1 g, 10.2 mmol) and osmium tetroxide (2 mL, 2% in water). The reaction mixture was stirred for three minutes and treated with a solution of sodium periodate (8.8 g, 4.0 mmol) in water (30 mL). The reaction mixture was stirred at room temperature overnight, acidified with 2 M hydrochloric acid (100 mL) and extracted with ethyl acetate (200 mL×3). The combined organic extracts were washed with water, brine, dried over magnesium sulfate and evaporated. The residue was purified by flash chromatography over silica (petroleum ether/ethyl acetate, v/v, 10/1) to afford the title compound as a yellow solid (3.5 g, 75%). MS: 453 m/z [M+H]⁺.

The following intermediates were prepared utilizing the procedures described for Intermediate 16.

| Inter. | | | |
|---|---|---|---|
| No. | Structure | Name | MS m/z [M + H]⁺ |
| 16-1 | 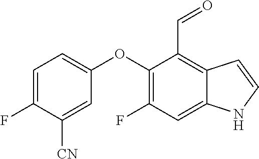 [SMILES: N#Cc1cc(Oc2c(F)cc3[nH]ccc3c2C=O)ccc1F] | 2-fluoro-5-((6-fluoro-4-formyl-1H-indol-5- yl)oxy)benzonitrile | 299 |
| 16-2 | 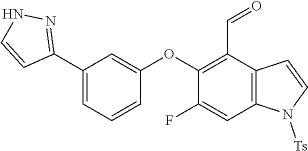 [SMILES: Cc1ccc(S(=O)(=O)n2ccc3c(C=O)c(Oc4cccc(-c5cc[nH]n5)c4)c(F)cc32)cc1] | 5-(3-(1H-pyrazol-3-yl)phenoxy)-6-fluoro-1- tosyl-1H-indole-4-carbaldehyde | 476 |

### Intermediate 17: 2-Fluoro-5-((6-fluoro-4-(hydroxymethyl)-1H-indol-5-yl)oxy)benzonitrile

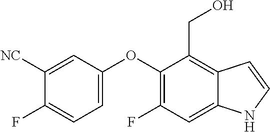 [SMILES: N#Cc1cc(Oc2c(F)cc3[nH]ccc3c2CO)ccc1F]

To a stirred solution of 2-fluoro-5-((6-fluoro-4-formyl-1H-indol-5-yl)oxy)benzonitrile (Intermediate 16-1, 8 g, 26.82 mmol) in ethanol (80 mL) was added sodium borohydride (1.52 g, 40.23 mmol) portion wise. The mixture was stirred at room temperature for 1 hour, quenched with water (100 mL) and extracted with ethyl acetate (3×50 mL). The combined organic extracts were washed with brine, dried over sodium sulfate, and concentrated. The residue was purified by silica gel column (80 g column, eluting with 0-80% ethyl acetate in petroleum ether) to give the title compound (3.8 g, 47%). MS (ESI): 323 m/z [M+Na]⁺, retention time: 1.83 minutes, purity: 98% (254 nm) (LC-MS method 2).

The following intermediate was prepared utilizing the procedures described for Intermediate 17.

| Inter. | | | |
|---|---|---|---|
| No. | Structure | Name | MS m/z [M + H]⁺ |
| 17-1 | 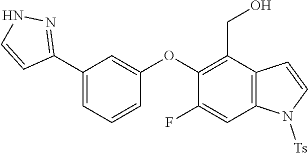 [SMILES: Cc1ccc(S(=O)(=O)n2ccc3c(CO)c(Oc4cccc(-c5cc[nH]n5)c4)c(F)cc32)cc1] | (5-(3-(1H-pyrazol-3-yl)phenoxy)-6-fluoro- 1-tosyl-1H-indol-4-yl)methanol | 478 |

### Intermediate 18: 4-Iodo-N,N-bis(4-methoxybenzyl)butane-1-sulfonamide

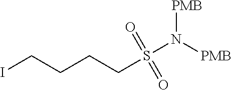

To a stirred solution of 4-chloro-N,N-bis(4-methoxybenzyl)butane-1-sulfonamide (Intermediate 18A, 3.4 g, 8.25 mmol) in acetone (50 mL) was added potassium iodide (4.1 g, 24.76 mmol). The reaction mixture was heated at 60° C. overnight and concentrated. The residue was dissolved in ethyl acetate (50 mL), the solution was washed with water, brine, dried over sodium sulfate, and concentrated. The crude product was purified by automated flash chromatography (40 g silica gel column, eluting with 0-20% ethyl acetate in petroleum ether) to give the title compound (3.6 g, 83%) as a yellow oil. MS (ESI): 526 m/z [M+Na]⁺, retention time: 2.17 minutes, purity: 90% (254 nm) (LC-MS method 4).

The following intermediate was prepared utilizing the procedures described for Intermediate 18.

| Inter. | | | |
|---|---|---|---|
| No. | Structure | Name | MS m/z [M + H]⁺ |
| 18-1 | 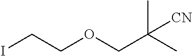 | 3-(2-iodoethoxy)-2,2- dimethylpropanenitrile | ¹H NMR (400 MHz, CDCl₃) δ 3.80 (t, J = 6.8 Hz, 2H), 3.45 (s, 2H), 3.28 (t, J = 6.8 Hz, 2H), 1.38 (s, 6H). |

### Intermediate 18A: 4-Chloro-N,N-bis(4-methoxybenzyl)butane-1-sulfonamide

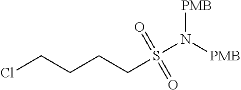

To a stirred and cooled (0° C.) solution of bis(4-methoxybenzyl)amine (2.1 g, 8.3 mmol) in dichloromethane (50 mL) was added triethylamine (1.7 g, 16.6 mmol) and 4-chlorobutane-1-sulfonyl chloride (2.4 g, 12.5 mmol). The reaction mixture was stirred at room temperature overnight, then diluted with dichloromethane (50 mL). The solution was washed with water, brine, dried over sodium sulfate, and concentrated. The residue was purified by automated flash chromatography (40 g silica gel column, eluting with 0-30% ethyl acetate in petroleum ether) to give the title compound (3.4 g, 95%) as yellow oil. MS (ESI): 434 m/z [M+Na]⁺, retention time: 2.11 minutes, purity: 90% (254 nm) (LC-MS method 4).

### Intermediate 19: 6-(N,N-Bis(4-methoxybenzyl)sulfamoyl)-2-phenylhexanoic acid

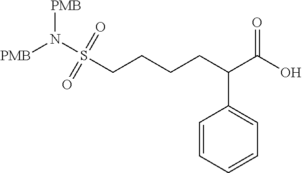

To a stirred solution of methyl 6-(N,N-bis(4-methoxybenzyl)sulfamoyl)-2-phenylhexanoate (Intermediate 19A, 1 g, 1.9 mmol) in tetrahydrofuran (10 mL) was added lithium hydroxide monohydrate (152 mg, 3.8 mmol). The reaction was stirred at room temperature for 16 hours, then acidified with 1 M hydrochloric acid to pH˜4 and extracted with ethyl acetate (2×50 mL). The combined organic extracts were washed with brine (50 mL×2), dried over sodium sulfate, and concentrated. The residue was purified by automated flash chromatography (12 g silica gel column, eluting with 5% methanol in dichloromethane) to give the title compound (920 mg, 95%) as oil. MS (ESI): 512 m/z [M+H]⁺, retention time: 2.01 minutes, purity: 92% (254 nm) (LC-MS method 4).

The following intermediates were prepared utilizing the procedures described for Intermediate 19.

| Inter. | | | |
|---|---|---|---|
| No. | Structure | Name | MS m/z [M + H]⁺ |
| 19-1 | 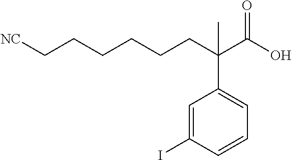 [SMILES: CC(CCCCCCC#N)(C(=O)O)c1cccc(I)c1] | 8-cyano-2-(3- iodophenyl)-2- methyloctanoic acid | 408 [M + Na]⁺ |
| 19-2 | 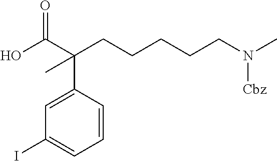 [SMILES: CN(CCCCCC(C)(C(=O)O)c1cccc(I)c1)C(=O)OCc1ccccc1] | (R)-7- (((benzyloxy)carbonyl) (methyl)amino)-2-(3- iodophenyl)-2- methylheptanoic acid | 510 |
| 19-3 | 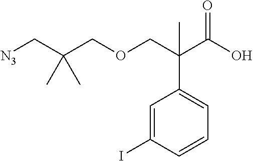 [SMILES: CC(C)(CN=[N+]=[N-])COCC(C)(C(=O)O)c1cccc(I)c1] | 3-(3-azido-2,2- dimethylpropoxy)-2-(3- iodophenyl)-2- methylpropanoic acid | 418 |
| 19-4 | 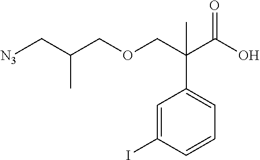 [SMILES: CC(CN=[N+]=[N-])COCC(C)(C(=O)O)c1cccc(I)c1] | 3-(3-azido-2- methylpropoxy)-2-(3- iodophenyl)-2- methylpropanoic acid | 426 [M + Na]⁺ |
| 19-5 | 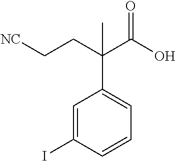 [SMILES: CC(CCC#N)(C(=O)O)c1cccc(I)c1] | 4-cyano-2-(3- iodophenyl)-2- methylbutanoic acid | 330 |
| 19-6 | 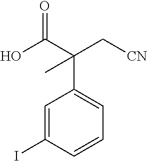 | 3-cyano-2-(3- iodophenyl)-2- methylpropanoic acid | 316 |
| 19-7 | 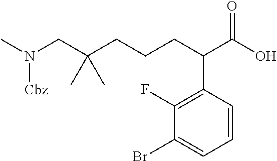 [SMILES: CN(CC(C)(C)CCCC(C(=O)O)c1cccc(Br)c1F)C(=O)OCc1ccccc1] | 7- (((benzyloxy)carbonyl) (methyl)amino)-2-(3-bromo- 2-fluorophenyl)-6,6- dimethylheptanoic acid | 494 |
| 19-8 | 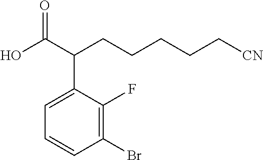 [SMILES: N#CCCCCCC(C(=O)O)c1cccc(Br)c1F] | 2-(3-bromo-2- fluorophenyl)-7- cyanoheptanoic acid | 350, 352 [M + Na]⁺ |
| 19-9 | 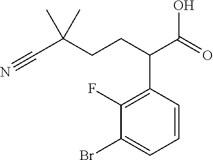 [SMILES: CC(C)(C#N)CCC(C(=O)O)c1cccc(Br)c1F] | 2-(3-bromo-2- fluorophenyl)-5-cyano-5- methylhexanoic acid | 350, 352 [M + Na]⁺ |
| 19-10 | 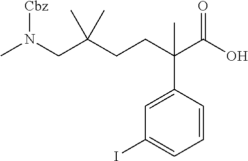 [SMILES: CN(CC(C)(C)CCC(C)(C(=O)O)c1cccc(I)c1)C(=O)OCc1ccccc1] | 6- (((benzyloxy)carbonyl) (methyl)amino)-2-(3- iodophenyl)-2,5,5- trimethylhexanoic acid | 546 [M + Na]⁺ |
| 19-11 | 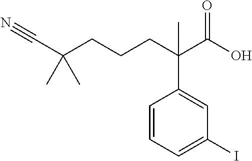 [SMILES: CC(C)(C#N)CCCC(C)(C(=O)O)c1cccc(I)c1] | 6-cyano-2-(3- iodophenyl)-2,6- dimethylheptanoic acid | 408 [M + Na]⁺ |
| 19-12 | 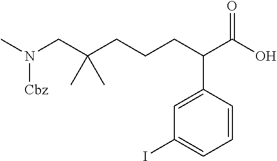 [SMILES: CN(CC(C)(C)CCCC(C(=O)O)c1cccc(I)c1)C(=O)OCc1ccccc1] | 7- (((benzyloxy)carbonyl) (methyl)amino)-2-(3- iodophenyl)-6,6- dimethylheptanoic acid | 546 [M + Na]⁺ |
| 19-13 | 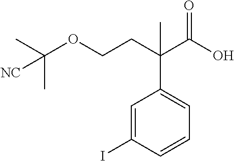 [SMILES: CC(C)(C#N)OCCC(C)(C(=O)O)c1cccc(I)c1] | 4-((2-cyanopropan-2- yl)oxy)-2-(3-iodophenyl)- 2-methylbutanoic acid | 410 [M + Na]⁺ |
| 19-14 | 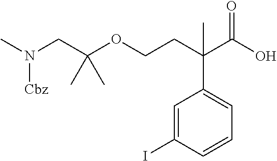 [SMILES: CN(CC(C)(C)OCCC(C)(C(=O)O)c1cccc(I)c1)C(=O)OCc1ccccc1] | 4-((1- (((benzyloxy)carbonyl) (methyl)amino)-2- methylpropan-2-yl)oxy)- 2-(3-iodophenyl)-2- methylbutanoic acid | 540 |
| 19-15 | 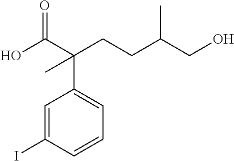 [SMILES: CC(CO)CCC(C)(C(=O)O)c1cccc(I)c1] | 6-hydroxy-2-(3- iodophenyl)-2,5- dimethylhexanoic acid | ¹H NMR (400 MHz, CD₃OD) δ 7.71 (s, 1H), 7.61 (d, J = 7.8 Hz, 1H), 7.39 (d, J = 7.8 Hz, 1H), 7.12 (t, J = 7.8 Hz, 1H), 3.33-3.32 (m, 2H), 2.02- 1.83 (m, 2H), 1.60-1.54 (m, 1H), 1.52 (s, 3H), 1.42-1.35 (m, 1H), 1.06- 0.97 (m, 1H), 0.95-0.89 (m, 3H) ppm. |
| 19-16 | 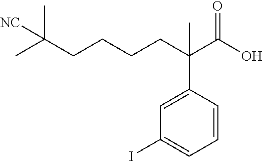 [SMILES: CC(C)(C#N)CCCCC(C)(C(=O)O)c1cccc(I)c1] | 7-cyano-2-(3- iodophenyl)-2,7- dimethyloctanoic acid | 354 [M − CO2]⁺ |
| 19-17 | 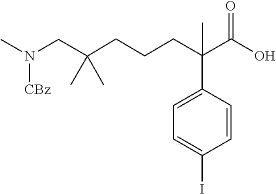 | 7- (((benzyloxy)carbonyl) (methyl)amino)-2-(4- iodophenyl)-2,6,6- trimethylheptanoic acid | 560 [M + Na]⁺ |
| 19-18 | 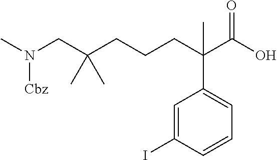 [SMILES: CN(CC(C)(C)CCCC(C)(C(=O)O)c1cccc(I)c1)C(=O)OCc1ccccc1] | 7- (((benzyloxy)carbonyl) (methyl)amino)-2-(3- iodophenyl)-2,6,6- trimethylheptanoic acid | 560 [M + Na]⁺ |
| 19-19 | 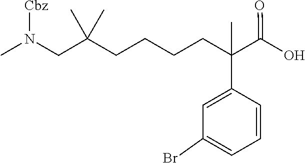 [SMILES: CN(CC(C)(C)CCCCC(C)(C(=O)O)c1cccc(Br)c1)C(=O)OCc1ccccc1] | 8- (((benzyloxy)carbonyl) (methyl)amino)-2-(3- bromophenyl)-2,7,7- trimethyloctanoic acid | 526, 528 [M + Na]⁺ |
| 19-20 | 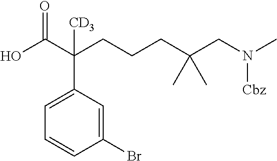 [SMILES: [2H]C([2H])([2H])C(CCCC(C)(C)CN(C)C(=O)OCc1ccccc1)(C(=O)O)c1cccc(Br)c1] | 7- (((benzyloxy)carbonyl) (methyl)amino)-2-(3- bromophenyl)-6,6- dimethyl-2-(methyl- d3)heptanoic acid | 493, 495 |
| 19-21 | 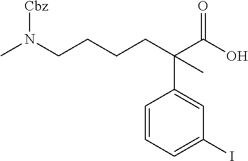 [SMILES: CN(CCCCC(C)(C(=O)O)c1cccc(I)c1)C(=O)OCc1ccccc1] | 6- (((benzyloxy)carbonyl) (methyl)amino)-2-(3- iodophenyl)-2- methylhexanoic acid | 496 |
| 19-22 | 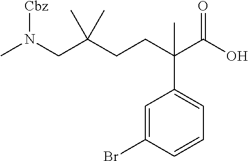 [SMILES: CN(CC(C)(C)CCC(C)(C(=O)O)c1cccc(Br)c1)C(=O)OCc1ccccc1] | 6- (((benzyloxy)carbonyl) (methyl)amino)-2-(3- bromophenyl)-2,5,5- trimethylhexanoic acid | 476, 478 |
| 19-23 | 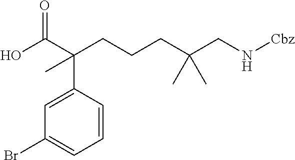 [SMILES: CC(C)(CCCC(C)(C(=O)O)c1cccc(Br)c1)CNC(=O)OCc1ccccc1] | 7- (((benzyloxy)carbonyl) amino)-2-(3-bromophenyl)- 2,6,6-trimethylheptanoic acid | 476, 478 |
| 19-24 | 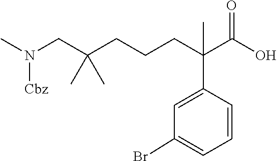 [SMILES: CN(CC(C)(C)CCCC(C)(C(=O)O)c1cccc(Br)c1)C(=O)OCc1ccccc1] | 7- (((benzyloxy)carbonyl) (methyl)amino)-2-(3- bromophenyl)-2,6,6- trimethylheptanoic acid | 512, 514 [M + Na]⁺ |
| 19-25 | 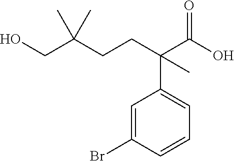 [SMILES: CC(C)(CO)CCC(C)(C(=O)O)c1cccc(Br)c1] | 2-(3-bromophenyl)-6- hydroxy-2,5,5- trimethylhexanoic acid | ¹H NMR (400 MHz, CDCl₃) δ 7.51 (t, J = 1.6 Hz, 1H), 7.40 (dt, J = 8.0, 1.6 Hz, 1H), 7.30 (dt, J = 8.0, 1.6 Hz, 1H), 7.21 (t, J = 8.0 Hz, 1H), 3.43 (d, J = 11.2 Hz, 1H), 3.28 (d, J = 11.2 Hz, 1H), 2.03-1.95 (m, 1H), 1.79- 1.73 (m, 1H), 1.57 (s, 3H), 1.22-1.15 (m, 2H), 0.87 (s, 3H), 0.82 (s, 3H) ppm. |
| 19-26 | 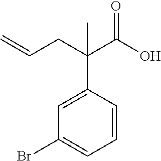 | 2-(3-bromophenyl)-2- methylpent-4-enoic acid | 269, 271 |
| 19-27 | 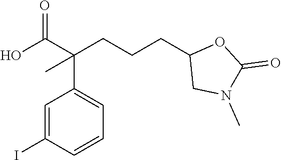 [SMILES: CN1CC(CCCC(C)(C(=O)O)c2cccc(I)c2)OC1=O] | 2-(3-iodophenyl)-2- methyl-5-(3-methyl-2- oxooxazolidin-5- yl)pentanoic acid | 418 |
| 19-28 | 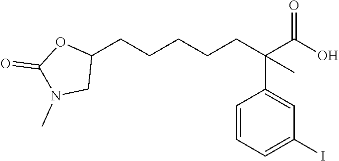 [SMILES: CN1CC(CCCCCC(C)(C(=O)O)c2cccc(I)c2)OC1=O] | 2-(3-iodophenyl)-2- methyl-7-(3-methyl-2- oxooxazolidin-5- yl)heptanoic acid | 446 |
| 19-29 | 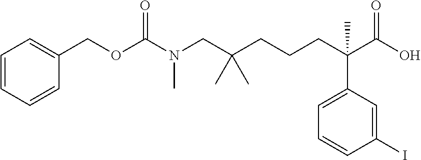 [SMILES: CN(CC(C)(C)CCC[C@@](C)(C(=O)O)c1cccc(I)c1)C(=O)OCc1ccccc1] | (R)-7- (((benzyloxy)carbonyl) (methyl)amino)-2-(3- iodophenyl)-2,6,6- trimethylheptanoic acid | 538 |
| 19-29 | 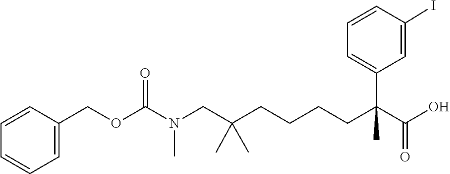 [SMILES: CN(CC(C)(C)CCCC[C@@](C)(C(=O)O)c1cccc(I)c1)C(=O)OCc1ccccc1] | (R)-8- (((benzyloxy)carbonyl) (methyl)amino)-2-(3- iodophenyl)-2,7,7- trimethyloctanoic acid | 552 |

### Intermediate 19A: Methyl 6-(N,N-bis(4-methoxybenzyl)sulfamoyl)-2-phenylhexanoate

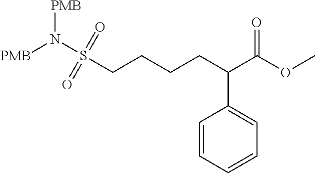

To a stirred and cooled (−78° C.) solution of methyl 2-phenylacetate (1.3 g, 8.5 mmol) in tetrahydrofuran (100 mL) was added lithium bis(trimethylsilyl)amide (4.3 mL, 2.1 mmol, 2 M in tetrahydrofuran). The reaction was stirred −78° C. for 1 hour, then treated with 4-iodo-N,N-bis(4-methoxybenzyl)butane-1-sulfonamide (Intermediate 18, 3.6 g, 7.1 mmol) at this temperature. The mixture was stirred at room temperature overnight, quenched with water, and acidified to pH˜5-6 with 2 M hydrochloric acid. The solution was extracted with ethyl acetate (3×40 mL). The combined organic layers were washed with brine, dried over sodium sulfate, and concentrated. The residue was purified by silica gel column chromatography (eluting with petroleum ether/ethyl acetate=0˜20%) to give the title compound (2.6 g, 64%) as oil. MS (ESI): 548 m/z [M+Na]⁺, retention time: 2.18 minutes, purity: 90% (254 nm) (LC-MS method 4).

The following intermediates were prepared utilizing the procedures described for Intermediate 19A.

| Inter. No. | Structure | Name | MS m/z [M + H]⁺ |
|---|---|---|---|
| 19A-1 | 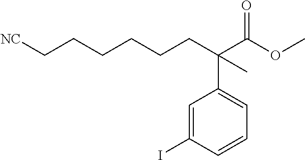 [SMILES: COC(=O)C(C)(CCCCCCC#N)c1cccc(I)c1] | methyl 8-cyano- 2-(3- iodophenyl)-2- methyloctanoate | 422 [M + Na]⁺ |
| 19A-2 | 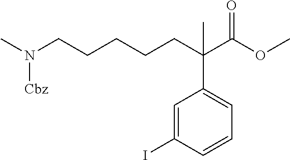 | methyl 7- (((benzyloxy) carbonyl)(methyl) amino)-2-(3- iodophenyl)-2- methylheptanoate | 524 |
| 19A-3 | 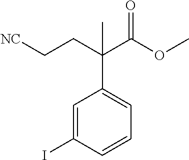 [SMILES: COC(=O)C(C)(CCC#N)c1cccc(I)c1] | methyl 4-cyano- 2-(3- iodophenyl)-2- methylbutanoate | MS: 344 m/z [M + H]⁺. ¹H NMR (400 MHz, CDCl₃) δ 7.63 (d, J = 7.6Hz, 1H), 7.58 (s, 1H), 7.23-7.20 (m, 1H), 7.11-7.06 (m, 1H), 3.71 (s, 3H), 2.35-2.20 (m, 4H), 1.60 (s, 3H) ppm. |
| 19A-4 | 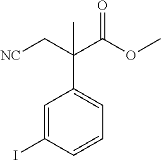 [SMILES: COC(=O)C(C)(CC#N)c1cccc(I)c1] | methyl 3-cyano- 2-(3- iodophenyl)-2- methylpropanoate | MS: 330 m/z [M + H]⁺. ¹H NMR (400 MHz, CDCl₃) δ 7.66-7.68 (m, 1H), 7.63 (s, 1H), 7.27-7.30 (m, 1H), 7.13-7.11 (m, 1H), 3.74 (s, 3H), 2.84-3.05 (m, 2H), 1.795 (s, 3H) ppm. |
| 19A-5 | 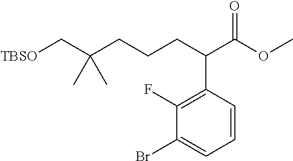 [SMILES: COC(=O)C(CCCC(C)(C)CO[Si](C)(C)C(C)(C)C)c1cccc(Br)c1F] | methyl 2-(3- bromo-2- fluorophenyl)-7- ((tert- butyldimethyl- silyl)oxy)-6,6- dimethyl- heptanoate | ¹H NMR (400 MHz, CDCl₃) δ 7.48-7.44 (m, 1H), 7.34-7.29 (m, 1H), 7.04-7.00 (m, 1H), 3.99 (t, J = 7.6 Hz, 1H), 3.70 (s, 3H), 3.19 (s, 2H), 2.08-2.02 (m, 1H), 1.77-1.71 (m, 1H), 1.28-1.18 (m, 4H), 0.891 (s, 9H), 0.80 (s, 3H), 0.78 (s, 3H), 0.01-0.02 (m, 6H)ppm |
| 19A-6 | 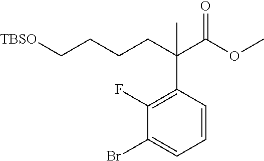 [SMILES: COC(=O)C(C)(CCCCO[Si](C)(C)C(C)(C)C)c1cccc(Br)c1F] | methyl 2-(3- bromo-2- fluorophenyl)-6- ((tert- butyldimethyl- silyl)oxy)-2- methylhexanoate | 447, 449 |
| 19A-8 | 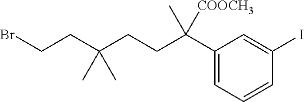 [SMILES: COC(=O)C(C)(CCC(C)(C)CCBr)c1cccc(I)c1] | methyl 7-bromo- 2-(3- iodophenyl)- 2,5,5- trimethyl- heptanoate | 489, 491 [M + Na]⁺ |
| 19A-9 | 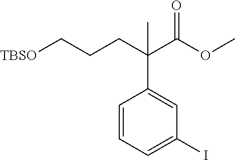 [SMILES: COC(=O)C(C)(CCCO[Si](C)(C)C(C)(C)C)c1cccc(I)c1] | methyl 5-((tert- butyldimethylsilyl) oxy)-2-(3- iodophenyl)-2- methylpentanoate | 463 |
| 19A-10 | 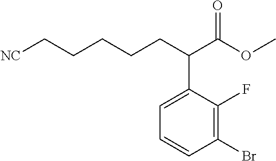 [SMILES: COC(=O)C(CCCCCC#N)c1cccc(Br)c1F] | methyl 2-(3- bromo-2- fluorophenyl)-7- cyanoheptanoate | 364, 366 [M + Na]⁺ |
| 19A-11 | 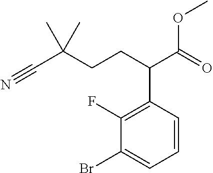 [SMILES: COC(=O)C(CCC(C)(C)C#N)c1cccc(Br)c1F] | methyl 2-(3- bromo-2- fluorophenyl)-5- cyano-5- methylhexanoate | 342, 344 |
| 19A-12 | 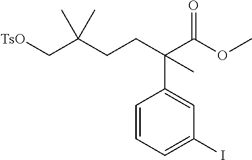 [SMILES: COC(=O)C(C)(CCC(C)(C)COS(=O)(=O)c1ccc(C)cc1)c1cccc(I)c1] | methyl 2-(3- iodophenyl)- 2,5,5-trimethyl- 6- (tosyloxy) hexanoate | 567 [M + Na]⁺ |
| 19A-13 | 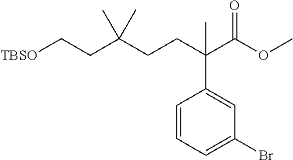 [SMILES: COC(=O)C(C)(CCC(C)(C)CCO[Si](C)(C)C(C)(C)C)c1cccc(Br)c1] | methyl 2-(3- bromophenyl)-7- ((tert- butyldimethylsilyl) oxy)-2,5,5- trimethyl- heptanoate | No UV absorption and no ionization using LC-Mass Method 7 between 0-3 minutes |
| 19A-14 | 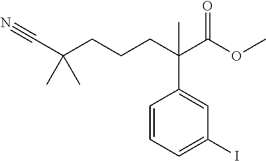 [SMILES: COC(=O)C(C)(CCCC(C)(C)C#N)c1cccc(I)c1] | methyl 6-cyano- 2-(3- iodophenyl)-2,6- dimethyl- heptanoate | 422 [M + Na]⁺ |
| 19A-15 | 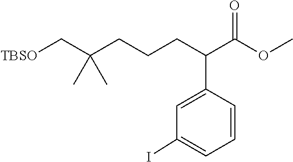 [SMILES: COC(=O)C(CCCC(C)(C)CO[Si](C)(C)C(C)(C)C)c1cccc(I)c1] | methyl 7-((tert- butyldimethylsilyl) oxy)-2-(3- iodophenyl)-6,6- dimethyl- heptanoate | ¹H NMR (400 MHz, CDCl₃) δ 7.70-7.51 (m, 2H), 7.28 (d, J = 7.7 Hz, 1H), 7.05 (t, J = 7.8 Hz, 1H), 3.67 (s, 3H), 3.56-3.41 (m, 1H), 3.25-3.08 (m, 2H), 2.02-1.68 (m, 2H), 1.22- 1.11 (m, 4H), 0.90-0.84 (m, 9H), 0.83-0.71 (m, 6H), 0.03-0.00 (m, 6H) ppm |
| 19A-16 | 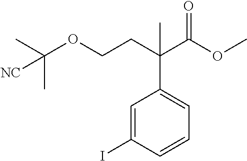 [SMILES: COC(=O)C(C)(CCOC(C)(C)C#N)c1cccc(I)c1] | methyl 4-((2- cyanopropan-2- yl)oxy)-2-(3- iodophenyl)-2- methylbutanoate | Mass: 424 m/z [M + Na]⁺. |
| 19A-17 | 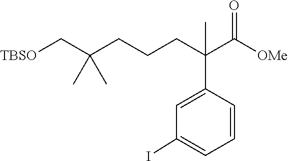 [SMILES: COC(=O)C(C)(CCCC(C)(C)CO[Si](C)(C)C(C)(C)C)c1cccc(I)c1] | methyl 7-((tert- butyldimethylsilyl) oxy)-2-(3- iodophenyl)- 2,6,6- trimethyl- heptanoate | 519 |
| 19A-18 | 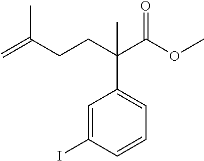 [SMILES: C=C(C)CCC(C)(C(=O)OC)c1cccc(I)c1] | methyl 2-(3- iodophenyl)-2,5- dimethylhex-5- enoate | ¹H NMR (400 MHz, CD₃OD) δ 7.55-7.52 (m, 1H), 7.50 (d, J = 7.8 Hz, 1H), 7.24-7.20 (m, 1H), 7.00 (t, J = 7.8 Hz, 1H), 4.61-4.53 (m, 2H), 3.56 (s, 3H), 2.02-1.92 (m, 2H), 1.80- 1.74 (m, 2H), 1.60 (s, 3H), 1.43 (s, 3H) ppm. |
| 19A-19 | 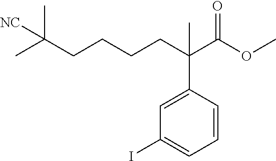 [SMILES: COC(=O)C(C)(CCCCC(C)(C)C#N)c1cccc(I)c1] | methyl 7-cyano- 2-(3- iodophenyl)-2,7- dimethyloctanoate | 436 [M + Na]⁺ |
| 19A-20 | 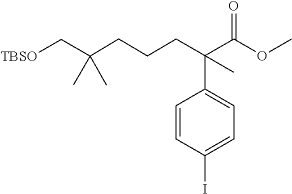 [SMILES: COC(=O)C(C)(CCCC(C)(C)CO[Si](C)(C)C(C)(C)C)c1ccc(I)cc1] | methyl 7-((tert- butyldimethylsilyl) oxy)-2-(4- iodophenyl)- 2,6,6- trimethyl- heptanoate | ¹HNMR (400 MHz, CDCl₃): δ 7.63-7.66 (m, 2H), 7.04-7.07 (m, 2H), 3.65 (s, 3H), 3.17 (s, 2H), 1.93-2.01 (m,1H), 1.82-1.86 (m, 1H), 1.52 (s, 3H), 1.17-1.24 (m, 2H), 1.06-1.14 (m, 2H), 0.882 (s, 9H), 0.78-0.76 (m, 6H), −0.008-0.000 (m, 6H) ppm. |
| 19A-21 | 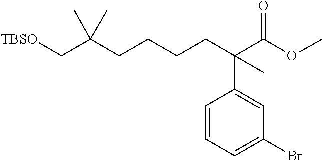 [SMILES: COC(=O)C(C)(CCCCC(C)(C)CO[Si](C)(C)C(C)(C)C)c1cccc(Br)c1] | methyl 2-(3- bromophenyl)-8- ((tert- butyldimethylsilyl) oxy)-2,7,7- trimethyloctanoate | ¹H NMR (400 MHz, CHCl₃) δ 7.45-7.44 (m, 1H), 7.38-7.36 (m, 1H), 7.22-7.18 (m, 2H), 3.66 (s, 3H), 3.20 (s, 2H), 2.07-1.83 (m, 2H), 1.52 (s, 3H), 1.22-1.17 (m, 6H), 0.88 (s, 9H), 0.78 (s, 6H), 0.02 (s, 6H) ppm. |
| 19A-22 | 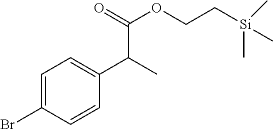 [SMILES: CC(C(=O)OCC[Si](C)(C)C)c1ccc(Br)cc1] | 2- (trimethylsilyl) ethyl 2-(4- bromophenyl) propanoate | ¹H NMR (400 MHz, CDCl₃) δ 7.42 (d, J = 8.3 Hz, 2H), 7.14 (d, J = 8.3 Hz, 2H), 4.20-4.07 (m, 2H), 3.55-3.51 (m, 1H), 1.51-1.47 (m, 3H), 1.01-0.89 (m, 2H), −0.01 (s, 9H) ppm. |
| 19A-23 | 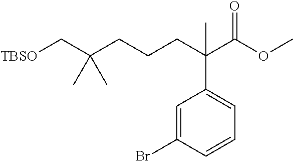 [SMILES: COC(=O)C(C)(CCCC(C)(C)CO[Si](C)(C)C(C)(C)C)c1cccc(Br)c1] | methyl 2-(3- bromophenyl)-7- ((tert- butyldimethylsilyl) oxy)-2,6,6- trimethyl- heptanoate | ¹H NMR (400 MHz, CDCl₃) δ 7.45 (s, 1H), 7.38-7.36 (m, 1H), 7.24-7.17 (m, 2H), 3.66 (s, 3H), 3.18 (s, 2H), 2.03-1.78 (m, 2H), 1.53 (s, 3H), 1.27-1.09 (m, 4H), 0.88 (s, 9H), 0.78 (s, 6H), 0.02 (s, 6H) ppm. |
| 19A-24 | 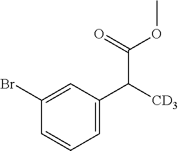 [SMILES: [2H]C([2H])([2H])C(C(=O)OC)c1cccc(Br)c1] | methyl 2-(3- bromophenyl) propanoate-3,3,3- d3 | 246, 248 |
| 19A-25 | 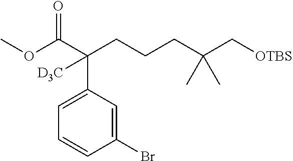 [SMILES: [2H]C([2H])([2H])C(CCCC(C)(C)CO[Si](C)(C)C(C)(C)C)(C(=O)OC)c1cccc(Br)c1] | methyl 2-(3- bromophenyl)-7- ((tert- butyldimethylsilyl) oxy)-6,6- dimethyl-2- (methyl- d3)heptanoate | ¹H NMR (400 MHz, CDCl₃) δ7.64 (s, 1H), 7.57 (d, J = 8.0 Hz, 1H), 7.26-7.25 (m, 1H), 7.05 (d, J = 8.0 Hz, 1H), 3.66 (s, 3H), 3.20 (s, 2H), 1.99-1.95 (m, 1H), 1.84-1.77 (m, 1H), 1.36-1.09 (m, 4H), 0.88 (s, 9H), 0.78 (s, 6H), 0.02 (s, 6H) ppm. |
| 19A-26 | 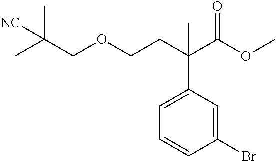 [SMILES: COC(=O)C(C)(CCOCC(C)(C)C#N)c1cccc(Br)c1] | methyl 2-(3- bromophenyl)-4- (2-cyano-2- methylpropoxy)- 2- methylbutanoate | 368, 370 |
| 19A-27 | 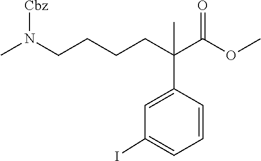 | methyl 6- (((benzyloxy) carbonyl)(methyl) amino)-2-(3- iodophenyl)-2- methylhexanoate | 532 [M + Na]⁺ |
| 19A-28 | 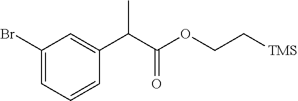 [SMILES: CC(C(=O)OCC[Si](C)(C)C)c1cccc(Br)c1] | 2- (trimethylsilyl) ethyl 2-(3- bromophenyl) propanoate | ¹H NMR (400 MHz, CDCl₃) δ 7.48-7.42 (m, 1H), 7.41-7.34 (m, 1H), 7.26-7.15 (m, 2H), 4.23-4.08 (m, 2H), 3.64 (q, J = 7.2 Hz, 1H), 1.49 (d, J = 7.2 Hz, 3H), 0.98-0.91 (m, 2H), −0.01 (s, 9H) ppm. |
| 19A-29 | 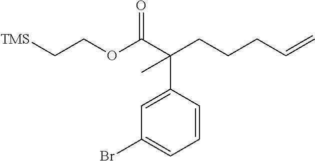 [SMILES: C=CCCCC(C)(C(=O)OCC[Si](C)(C)C)c1cccc(Br)c1] | 2- (trimethylsilyl) ethyl 2-(3- bromophenyl)-2- methylhept-6- enoate | ¹H NMR (400 MHz, CDCl₃) δ 7.51-7.45 (m, 1H), 7.42-7.34 (m, 1H), 7.27-7.16 (m, 2H), 5.82-5.74 (m, 1H), 5.08-4.92 (m, 2H), 4.23- 4.10 (m, 2H), 2.14-1.77 (m, 4H), 1.54 (s, 3H), 1.34-1.22 (m, 2H), 0.98-0.88 (m, 2H), 0.02 (s, 9H) ppm. |
| 19A-30 | 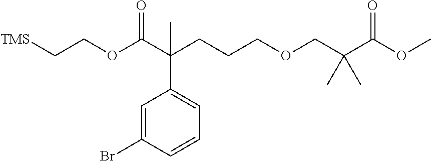 [SMILES: COC(=O)C(C)(C)COCCCC(C)(C(=O)OCC[Si](C)(C)C)c1cccc(Br)c1] | 2- (trimethylsilyl) ethyl 2-(3- bromophenyl)-5- (3-methoxy-2,2- dimethyl-3- oxopropoxy)-2- methylpentanoate | 523, 525 [M + Na]⁺ |
| 19A-31 | 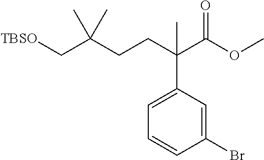 [SMILES: COC(=O)C(C)(CCC(C)(C)CO[Si](C)(C)C(C)(C)C)c1cccc(Br)c1] | methyl 2-(3- bromophenyl)-6- ((tert- butyldimethylsilyl) oxy)-2,5,5- trimethyl- hexanoate | ¹H NMR (400 MHz, CDCl₃) δ 7.44 (t, J = 1.6 Hz, 1H), 7.36 (dt, J = 7.2, 1.6 Hz, 1H), 7.23- 7.16 (m, 2H), 3.65 (s, 3H), 3.20 (s, 2H), 2.01- 1.82 (m, 2H), 1.50 (s, 3H), 1.11-1.01 (m, 2H), 0.87 (s, 9H), 0.81 (s, 3H), 0.80 (s, 3H), 0.00 (s, 6H) ppm. |
| 19A-32 | 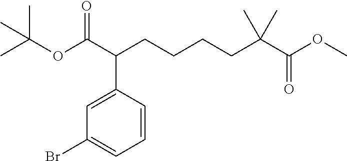 [SMILES: COC(=O)C(C)(C)CCCCC(C(=O)OC(C)(C)C)c1cccc(Br)c1] | 8-(tert-butyl) 1- methyl 7-(3- bromophenyl)- 2,2- dimethyloctane- dioate | 449, 451 [M + Na]⁺ |
| 19A-33 | 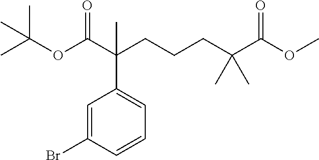 [SMILES: COC(=O)C(C)(C)CCCC(C)(C(=O)OC(C)(C)C)c1cccc(Br)c1] | 1-(tert-butyl) 7- methyl 2-(3- bromophenyl)- 2,6,6- trimethyl- heptanedioate | 449, 451 [M + Na]⁺ |
| 19A-34 | 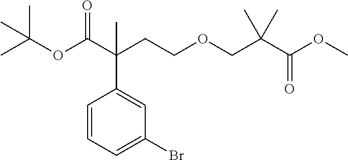 [SMILES: COC(=O)C(C)(C)COCCC(C)(C(=O)OC(C)(C)C)c1cccc(Br)c1] | tert-butyl 2-(3- bromophenyl)-4- (3-methoxy-2,2- dimethyl-3- oxopropoxy)-2- methylbutanoate | 465, 467 [M + Na]⁺ |
| 19A-35 | 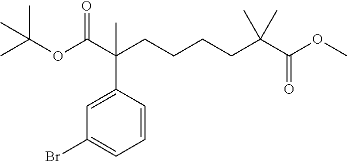 [SMILES: COC(=O)C(C)(C)CCCCC(C)(C(=O)OC(C)(C)C)c1cccc(Br)c1] | 1-(tert-butyl) 8- methyl 2-(3- bromophenyl)- 2,7,7- trimethyloctane dioate | 463, 465 [M + Na]⁺ |
| 19A-36 | 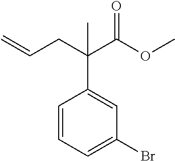 [SMILES: C=CCC(C)(C(=O)OC)c1cccc(Br)c1] | methyl 2-(3- bromophenyl)-2- methylpent-4- enoate | 283, 285 |
| 19A-37 | 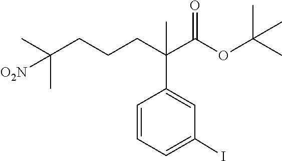 [SMILES: CC(C)(C)OC(=O)C(C)(CCCC(C)(C)[N+](=O)[O-])c1cccc(I)c1] | tert-butyl 2-(3- iodophenyl)-2,6- dimethyl-6- nitroheptanoate | 484 [M + Na]⁺ |
| 19A-38 | 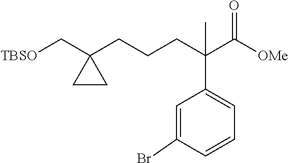 [SMILES: COC(=O)C(C)(CCCC1(CO[Si](C)(C)C(C)(C)C)CC1)c1cccc(Br)c1] | methyl 2-(3- bromophenyl)-5- (1-(((tert- butyldimethylsilyl) oxy)methyl)cyclo- propyl)-2- methylpentanoate | 469, 471 |
| 19A-39 | 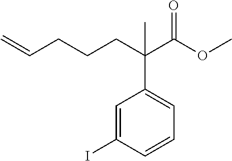 [SMILES: C=CCCCC(C)(C(=O)OC)c1cccc(I)c1] | methyl 2-(3- iodophenyl)-2- methylhept-6- enoate | 359 |
| 19A-40 | 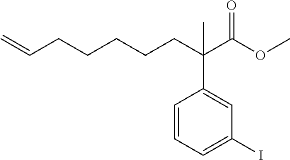 [SMILES: C=CCCCCCC(C)(C(=O)OC)c1cccc(I)c1] | methyl 2-(3- iodophenyl)-2- methylnon-8- enoate | 387 |

### Intermediate 20: 7-Bromo-6-oxo-5-phenylheptane-1-sulfonamide

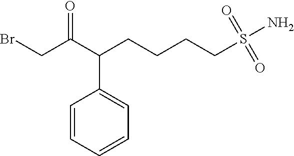 [SMILES: NS(=O)(=O)CCCCC(C(=O)CBr)c1ccccc1]

A solution of 7-bromo-N,N-bis(4-methoxybenzyl)-6-oxo-5-phenylheptane-1-sulfonamide (Intermediate 1-8, 680 mg, 1.15 mmol) in trifluoroacetic acid (10 mL) was stirred at room temperature for 1 hour. The mixture was concentrated to give the crude title compound (360 mg, 90%) as an oil, which was used for the next step without further purification. MS (ESI): 348, 350 m/z [M+H]⁺, retention time: 1.89 minutes, purity: 90% (254 nm) (LC-MS method 4).

### Intermediate 21: Ethyl 3-(5-(3-carbamimidoyl-4-fluorophenoxy)-6-fluoro-1H-indol-4-yl)propanoate

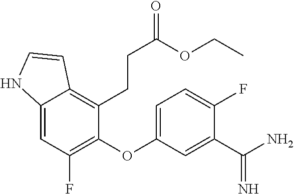 [SMILES: CCOC(=O)CCc1c(Oc2ccc(F)c(C(=N)N)c2)c(F)cc2[nH]ccc12]

To a solution of ethyl (E)-3-(5-(3-carbamimidoyl-4-fluorophenoxy)-6-fluoro-1H-indol-4-yl)acrylate (Intermediate 5-2, 1.39 g; 3.6 mmol) in tetrahydrofuran (25 mL) was added acetic acid (1 mL) and 10% Pd on carbon (50% wet, 0.3 g). The mixture was stirred at room temperature under hydrogen balloon for 16 hours, filtered through a pad of Celite. The filter cake was rinsed with tetrahydrofuran (25 mL). The combined filtrate was concentrated. The residue was dissolved in ethyl acetate (150 mL), washed with sodium bicarbonate, brine, dried over sodium sulfate, and concentrated to give the title compound (1.3 g; 94%) as a solid. MS (ESI): 388 m/z [M+H]⁺, retention time: 1.54 minutes, purity: 63% (254 nm) (LC-MS method 5).

### Intermediate 22: Methyl 6-azido-5,5-difluoro-2-(3-iodophenyl)hexanoate

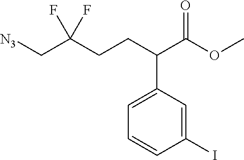 [SMILES: COC(=O)C(CCC(F)(F)CN=[N+]=[N-])c1cccc(I)c1]

To a stirred and cooled (0° C.) solution of methyl 6-azido-2-(3-iodophenyl)-5-oxohexanoate (Intermediate 22C, 2.7 g, 7 mmol) in dichloromethane (50 mL) was added diethylaminosulfur trifluoride (3.3 g, 21 mmol) portion wise. The reaction mixture was stirred at room temperature for 16 hours, then diluted with dichloromethane (150 mL). The solution was washed with water (80 mL), saturated sodium bicarbonate, brine, dried over sodium sulfate, and concentrated. The residue was purified by automated silica gel column (40 g column, eluting with 0-18% ethyl acetate in petroleum ether) to give the title compound (2.0 g, 70%) as oil. MS (ESI): 432 m/z [M+Na]⁺, retention time: 2.10 minutes, purity: 98% (254 nm) (LC-MS method 7).

### Intermediate 22A: Methyl 6-bromo-5-hydroxy-2-(3-iodophenyl)hexanoate

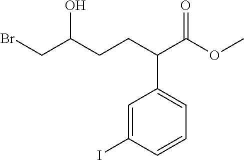 [SMILES: COC(=O)C(CCC(O)CBr)c1cccc(I)c1]

To a stirred and cooled (0° C.) solution of methyl 2-(3-iodophenyl)hex-5-enoate (Intermediate 15A-1, 4.95 g, 15 mmol) in dimethyl sulfoxide (25 mL) was added water (540 μL, 30 mmol) and N-bromosuccinimide (2.94 g, 16.5 mmol). The mixture was stirred at 0° C. for 2 hours. The reaction mixture was diluted with ethyl acetate (100 mL). The solution was washed with saturated sodium bicarbonate, brine, dried over sodium sulfate, and concentrated. The residue was purified by automated flash chromatography (80 g silica gel column, eluting with 0-30% ethyl acetate in petroleum ether) to give the title compound (4.1 g; 69.6%) as an oil. MS (ESI): 427, 429 m/z [M+H]⁺, retention time: 2.00 minutes, purity: >99% (254 nm) (LC-MS method 7).

The following intermediate was prepared utilizing the procedures described for Intermediate 22A.

| Inter. No. | Structure | Name | MS m/z [M + H]⁺ |
|---|---|---|---|
| 22A-1 | 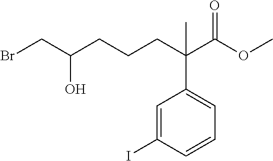 [SMILES: COC(=O)C(C)(CCCC(O)CBr)c1cccc(I)c1] | methyl 7-bromo-6-hydroxy-2-(3- iodophenyl)-2-methylheptanoate | 455, 457 |

### Intermediate 22B: Methyl 6-bromo-2-(3-iodophenyl)-5-oxohexanoate

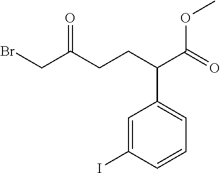 [SMILES: COC(=O)C(CCC(=O)CBr)c1cccc(I)c1]

To a solution of methyl 6-bromo-5-hydroxy-2-(3-iodophenyl)hexanoate (Intermediate 22A, 4.05 g, 9.5 mmol) in dichloromethane (80 mL) was added Dess-Martin periodinane (4.46 g, 10.5 mmol). The mixture was stirred at room temperature for 2 hours and filtered. The filter cake was rinsed with dichloromethane (100 mL). The combined filtrate was concentrated. The residue was purified by automated flash chromatography (40 g silica gel column, eluting with 0-30% ethyl acetate in petroleum ether) to give the title compound (2.7 g, 68%) as an oil. MS (ESI): 425, 427 m/z [M+H]⁺, retention time: 2.06 minutes, purity: >99% (254 nm) (LC-MS method 7).

### Intermediate 22C: Methyl 6-azido-2-(3-iodophenyl)-5-oxohexanoate

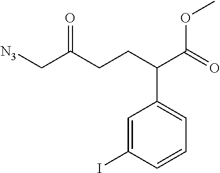 [SMILES: COC(=O)C(CCC(=O)CN=[N+]=[N-])c1cccc(I)c1]

To a stirred and cooled (0° C.) solution of sodium azide (Intermediate 22B, 0.74 g, 11.4 mmol) in deionized water (3.2 mL) was added dropwise a solution of methyl 6-bromo-2-(3-iodophenyl)-5-oxohexanoate (3.23 g. 7.6 mmol) in tetrahydrofuran (32 mL). The yellow-colored solution was stirred for 2 hours at room temperature and extracted with ethyl acetate (3×45 mL). The combined organic phases were washed with water, brine, dried over sodium sulfate, and concentrated. The residue was purified by automated flash chromatography (40 g silica gel column, eluting with 0-20% ethyl acetate in petroleum ether) to give the title compound (2.4 g, 82%) as oil. MS (ESI): 388 m/z [M+H]⁺, retention time: 2.04 minutes, purity: >99% (254 nm) (LC-MS method 7).

The following intermediates were prepared utilizing the procedures described for Intermediate 22C.

| Inter. No. | Structure | Name | MS m/z [M + H]⁺ |
|---|---|---|---|
| 22C-1 | 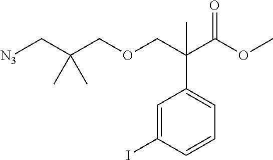 [SMILES: COC(=O)C(C)(COCC(C)(C)CN=[N+]=[N-])c1cccc(I)c1] | methyl 3-(3-azido-2,2-dimethylpropoxy)-2- (3-iodophenyl)-2-methylpropanoate | 454 [M + Na]⁺ |
| 22C-2 | 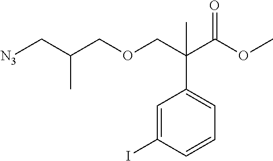 [SMILES: COC(=O)C(C)(COCC(C)CN=[N+]=[N-])c1cccc(I)c1] | methyl 3-(3-azido-2-methylpropoxy)-2-(3- iodophenyl)-2-methylpropanoate | 440 [M + Na]⁺ |

### Intermediate 23: 2-Fluoro-5-((6-fluoro-4-(2-hydroxyethyl)-1-tosyl-1H-indol-5-yl)oxy)benzonitrile

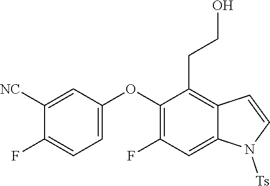 [SMILES: Cc1ccc(S(=O)(=O)n2ccc3c(CCO)c(Oc4ccc(F)c(C#N)c4)c(F)cc32)cc1]

To a stirred and cooled (0° C.) solution of 2-fluoro-5-((6-fluoro-4-(2-oxoethyl)-1-tosyl-1H-indol-5-yl)oxy)benzonitrile (Intermediate 7B, 3.2 g, 6.88 mmol in methanol (40 mL) was added sodium borohydride (781 mg, 20.64 mmol). The mixture was stirred at room temperature for 1 hour, quenched with water (20 mL) and concentrated to remove methanol. The aqueous residue was extracted with ethyl acetate (2×50 mL). The combined organic phases were washed with brine, dried over sodium sulfate, and concentrated. The residue was purified by silica gel column chromatography (petroleum ether/ethyl acetate=1:1) to give the title compound (3.0 g, 97%) as a white solid. MS: 469 m/z [M+H]⁺.

### Intermediate 24: 5-((4-(2-Azidoethyl)-6-fluoro-1-tosyl-1H-indol-5-yl)oxy)-2-fluorobenzonitrile

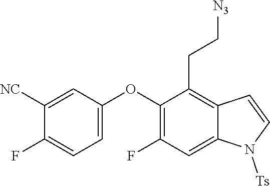 [SMILES: Cc1ccc(S(=O)(=O)n2ccc3c(CCN=[N+]=[N-])c(Oc4ccc(F)c(C#N)c4)c(F)cc32)cc1]

To a stirred solution of 2-(5-(3-cyano-4-fluorophenoxy)-6-fluoro-1-tosyl-1H-indol-4-yl)ethyl methanesulfonate (Intermediate 24A, 3.6 g, 6.59 mmol) in dimethylformamide (40 mL) was added sodium azide (1.3 g, 19.77 mmol). The mixture was heated at 70° C. for 2 hours, then cooled to room temperature. The mixture was partitioned between water (50 mL) and ethyl acetate (50 mL). The separated organic phase, combined with two additional ethyl acetate extracts (2×50 mL), was washed with water, brine, dried over sodium sulfate, and concentrated. The residue was purified by silica gel column chromatography (petroleum ether/ethyl acetate=2:1) to give the title compound (2.9 g, 89%) as a white solid. MS: 516 m/z [M+Na]⁺.

The following intermediates were prepared utilizing the procedures described for Intermediate 24.

| Inter. No. | Structure | Name | MS m/z [M + H]⁺ |
|---|---|---|---|
| 24-1 | 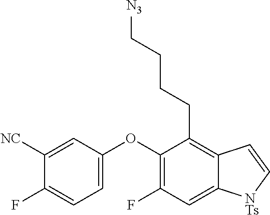 [SMILES: Cc1ccc(S(=O)(=O)n2ccc3c(CCCCN=[N+]=[N-])c(Oc4ccc(F)c(C#N)c4)c(F)cc32)cc1] | 5-((4-(4-azidobutyl)- 6-fluoro-1-tosyl-1H- indol-5-yl)oxy)-2- fluorobenzonitrile | 544 [M + Na]⁺ |
| 24-2 | 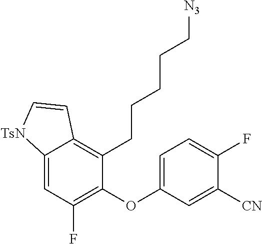 [SMILES: Cc1ccc(S(=O)(=O)n2ccc3c(CCCCCN=[N+]=[N-])c(Oc4ccc(F)c(C#N)c4)c(F)cc32)cc1] | 5-((4-(5- azidopentyl)-6- fluoro-1-tosyl-1H- indol-5-yl)oxy)-2- fluorobenzonitrile | MS: 558 m/z [M + Na]⁺. ¹H NMR (400 MHz, Acetone-d₆) δ 7.98 (d, J = 8.8 Hz, 2H), 7.84- 7.79 (m, 2H), 7.47-7.39 (m, 3H), 7.38-7.32 (m, 2H), 6.98 (d, J = 3.6 Hz, 1H), 3.25-3.20 (m, 2H), 2.85-2.80 (m, 2H), 2.40 (s, 3H), 1.60-1.50 (m, 4H), 1.39-1.33 (m, 2H) ppm |
| 24-3 | 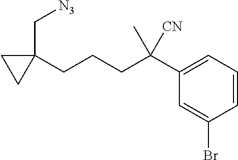 [SMILES: CC(C#N)(CCCC1(CN=[N+]=[N-])CC1)c1cccc(Br)c1] | 5-(1- (azidomethyl)cyclo- propyl)-2-(3- bromophenyl)-2- methylpentanenitrile | 369, 371 [M + Na]⁺ |
| 24-4 | 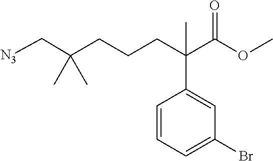 [SMILES: COC(=O)C(C)(CCCC(C)(C)CN=[N+]=[N-])c1cccc(Br)c1] | methyl 7-azido-2-(3- bromophenyl)- 2,6,6- trimethylheptanoate | 404, 407 [M + Na]⁺ |
| 24-5 | 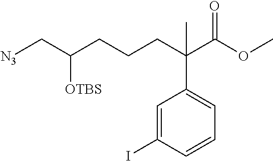 [SMILES: COC(=O)C(C)(CCCC(CN=[N+]=[N-])O[Si](C)(C)C(C)(C)C)c1cccc(I)c1] | methyl 7-azido-6- ((tert- butyldimethylsilyl) oxy)-2-(3- iodophenyl)-2- methylheptanoate | 554 [M + Na]⁺ |

### Intermediate 24A: 2-(5-(3-Cyano-4-fluorophenoxy)-6-fluoro-1-tosyl-1H-indol-4-yl)ethyl methanesulfonate

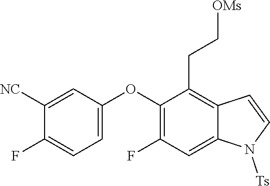 [SMILES: Cc1ccc(S(=O)(=O)n2ccc3c(CCOS(C)(=O)=O)c(Oc4ccc(F)c(C#N)c4)c(F)cc32)cc1]

To a stirred and cooled (0° C.) solution of 2-fluoro-5-((6-fluoro-4-(2-hydroxyethyl)-1-tosyl-1H-indol-5-yl)oxy)benzonitrile (Intermediate 23, 3.0 g, 6.40 mmol) and triethylamine (1.9 g, 19.20 mmol) in tetrahydrofuran (100 mL) was added methanesulfonyl chloride (1.5 g, 12.80 mmol). The mixture was stirred at room temperature for 1 hour, diluted with water and extracted with ethyl acetate (3×50 mL). The combined organic phases were washed with brine, dried over sodium sulfate, and concentrated to give the title compound (3.6 g, 100%) as yellow solid. MS: 547 m/z [M+H]⁺.

### Intermediate 25: Benzyl (5-iodopentyl)(methyl)carbamate

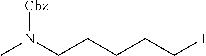 [SMILES: CN(CCCCCI)C(=O)OCc1ccccc1]

To a stirred solution of benzyl (5-bromopentyl)(methyl)carbamate (Intermediate 25A, 10.0 g, 31.9 mmol) in acetone (150 mL) was added sodium iodide (14.4 g, 95.7 mmol)). The mixture was stirred at 80° C. overnight and concentrated. The residue was purified by silica gel column chromatography (eluting with petroleum ether/ethyl acetate=4:1) to give the title compound (9.0 g, 81%) as a yellow oil. MS (ESI): 362 m/z [M+H]⁺, retention time: 2.08 minutes, purity: 96% (214 nm) (LC-MS method 7).

The following intermediate was prepared utilizing the procedures described for Intermediate 25.

| Inter. No. | Structure | Name | MS m/z [M + H]⁺ |
|---|---|---|---|
| 25-1 | 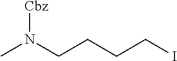 [SMILES: CN(CCCCI)C(=O)OCc1ccccc1] | benzyl (4-iodobutyl)(methyl)carbamate | 370 [M + Na]⁺ |

### Intermediate 25A: Benzyl (5-bromopentyl)(methyl)carbamate

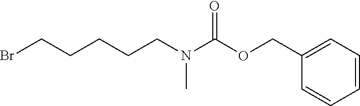 [SMILES: CN(CCCCCBr)C(=O)OCc1ccccc1]

To a stirred and cooled (0° C.) suspension of sodium hydride (4.8 g, 120 mmol) in N,N-dimethylformamide (150 mL) was added a solution of benzyl methylcarbamate (16.5 g, 100 mmol) and 1,5-dibromopentane (69 g, 300 mmol) in N,N-dimethylformamide (50 ml). The reaction mixture was warmed to room temperature and stirred for 4 hours, quenched with water (250 mL), and extracted with ethyl acetate (3×100 mL). The combined organic phases were washed with brine, dried over sodium sulfate and concentrated. The residue was purified by silica gel column chromatography (eluting with ethyl acetate/petroleum ether (20:80)) to afford the title compound (18.2 g, 58%) as an oil. MS (ESI): 314 m/z [M+H]⁺, retention time: 2.20 minutes, purity: 95% (214 nm) (LC-MS method 7).

The following intermediate was prepared utilizing the procedures described for Intermediate 25A.

| Inter. No. | Structure | Name | MS m/z [M + H]⁺ |
|---|---|---|---|
| 25A-1 | 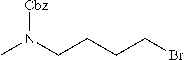 [SMILES: CN(CCCCBr)C(=O)OCc1ccccc1] | benzyl (4-bromobutyl)(methyl)carbamate | 300, 302 |

### Intermediate 26: Methyl 3-(5-(3-cyano-4-fluorophenoxy)-1H-indol-4-yl)-2,2-dimethylpropanoate

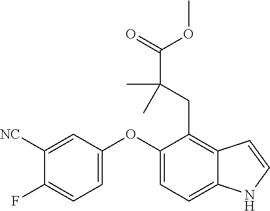 [SMILES: COC(=O)C(C)(C)Cc1c(Oc2ccc(F)c(C#N)c2)ccc2[nH]ccc12]

To a solution of methyl 3-(5-(3-cyano-4-fluorophenoxy)-6-fluoro-1-tosyl-1H-indol-4-yl)-2,2-dimethylpropanoate (Intermediate 26C, 1.8 g, 3.34 mmol) in methanol (20 mL) was added potassium carbonate (923 mg, 6.68 mmol), the mixture was stirred at 70° C. for 3 hours and concentrated. The residue was quenched with water (100 mL), extracted with ethyl acetate (3×50 mL). The combined organic extracts were dried over sodium sulfate and concentrated. The crude product was purified by flash chromatography (eluting with 0-40% ethyl acetate in hexane to give the title compound (577 mg, 44%) as a white solid. MS (ESI): 389 m/z [M+Na]⁺, purity: 98% (214 nm) (LC-MS method 5).

### Intermediate 26A: 2-Fluoro-5-((6-fluoro-4-(hydroxymethyl)-1-tosyl-1H-indol-5-yl)oxy)benzonitrile

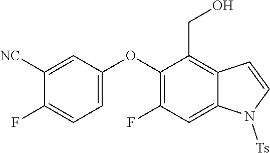 [SMILES: Cc1ccc(S(=O)(=O)n2ccc3c(CO)c(Oc4ccc(F)c(C#N)c4)c(F)cc32)cc1]

To a stirred solution of methyl 5-(3-cyano-4-fluorophenoxy)-6-fluoro-1-tosyl-1H-indole-4-carboxylate (Intermediate 3-1, 5 g, 9.49 mmol) in 50 mL of tetrahydrofuran was added lithium borohydride (626 mg, 28.46 mmol). The mixture was stirred at room temperature for 30 min and then refluxed 2 hours, cooled to room temperature, quenched with saturated ammonium chloride. The solution was extracted with ethyl acetate (3×50 mL), dried over sodium sulfate, and concentrated. The residue was purified by silica gel chromatography (eluting with 0-50% ethyl acetate in heptanes to give the title compound (1.76 g, 27%) as a yellow solid. MS (ESI): 477 m/z [M+Na]⁺, purity: 88% (214 nm) (LC-MS method 5).

### Intermediate 26B: 5-((4-(Bromomethyl)-6-fluoro-1-tosyl-1H-indol-5-yl)oxy)-2-fluorobenzonitrile

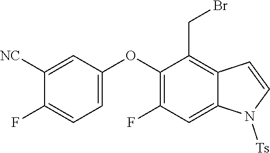 [SMILES: Cc1ccc(S(=O)(=O)n2ccc3c(CBr)c(Oc4ccc(F)c(C#N)c4)c(F)cc32)cc1]

To a stirred and cooled (0° C.) solution of 2-fluoro-5-((6-fluoro-4-(hydroxymethyl)-1-tosyl-1H-indol-5-yl)oxy)benzonitrile (Intermediate 26A, 5.5 g, 12 mmol) and triphenylphosphine (4.76 g, 18.2 mmol) in 100 mL of dichloromethane was added N-bromosuccinimide (3.23 g, 18.2 mmol). The mixture was stirred at 0° C. for 30 minutes, then room temperature for 2 hours. The reaction was quenched with 200 mL of water at 0° C., extracted with dichloromethane (3×50 mL). The combined organic layers were dried over sodium sulfate and concentrated. The residue was purified by silica gel chromatography (eluting with 0-20% ethyl acetate in heptanes) to give the title compound (4.75 g, 76%) as a white solid. MS (ESI): 517, 519 m/z [M+H]⁺, purity: 53% (214 nm) (LC-MS method 5).

### Intermediate 26C: Methyl 3-(5-(3-cyano-4-fluorophenoxy)-6-fluoro-1-tosyl-1H-indol-4-yl)-2,2-dimethylpropanoate

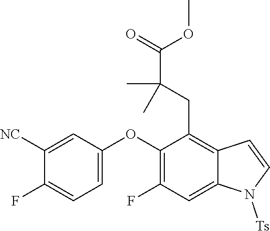 [SMILES: COC(=O)C(C)(C)Cc1c(Oc2ccc(F)c(C#N)c2)c(F)cc2c1ccn2S(=O)(=O)c1ccc(C)cc1]

To a stirred and cooled (−78° C.) solution of diisopropylamine (2.94 g, 29 mmol) in tetrahydrofuran (50 mL) was added n-butyllithium (2.5 M in hexanes, 11.6 mL, 29 mmol) dropwise. The mixture was stirred at this temperature for 30 minutes, then treated with methyl isobutyrate (2.97 g, 29 mmol). The mixture was then warmed up to 0° C. and stirred at 0° C. for 30 minutes, and re-cooled to −78° C., and treated with a solution of 5-((4-(bromomethyl)-6-fluoro-1-tosyl-1H-indol-5-yl)oxy)-2-fluorobenzonitrile (Intermediate 26B, 3 g, 5.8 mmol) in 5 mL of tetrahydrofuran dropwise. The solution was warmed to room temperature and stirred for 2 hours, quenched with saturated ammonium chloride, and extracted with ethyl acetate (3×50 mL). The combined organic phases were dried over sodium sulfate and concentrated. The residue was purified by flash chromatography (eluting with 0-20% ethyl acetate in hexane) to give the title compound (2.1 g, 67%) as a white solid. MS (ESI): 539 m/z [M+H]⁺, retention time: 2.26 minutes, purity: >99% (214 nm) (LC-MS method 5).

### Intermediate 27: Benzyl (3-(2-(3-iodophenyl)-2-methyl-3-oxobutoxy)-2,2-dimethylpropyl)carbamate

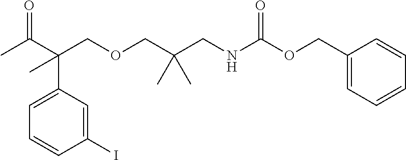 [SMILES: CC(=O)C(C)(COCC(C)(C)CNC(=O)OCc1ccccc1)c1cccc(I)c1]

To a stirred solution of 4-(3-amino-2,2-dimethylpropoxy)-3-(3-iodophenyl)-3-methylbutan-2-one (Intermediate 27B, 4.2 g, 10.80 mmol) in tetrahydrofuran (50 mL) was added benzyl chloroformate (2.75 g, 16.20 mmol) and triethylamine (2.2 g, 21.60 mmol). The mixture was stirred at 0° C. for 2 hours, then quenched with 60 mL of water, and extracted with ethyl acetate (3×60 mL). The combined organic layers were dried over sodium sulfate and concentrated, The residue was purified by silica gel column chromatography (eluting with petroleum ether/ethyl acetate=3/1) to give the title compound (2.8 g, 60% for three steps) as light-yellow liquid. MS-ESL: 524.1 m/z [M+H]⁺.

The following intermediates were prepared utilizing the procedures described for Intermediate 27.

| Inter. No. | Structure | Name | MS m/z [M + H]⁺ |
|---|---|---|---|
| 27-1 | 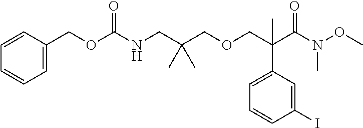 [SMILES: CON(C)C(=O)C(C)(COCC(C)(C)CNC(=O)OCc1ccccc1)c1cccc(I)c1] | benzyl (3-(2-(3-iodo- phenyl)-3-(methoxy- (methyl)amino)-2- methyl-3-oxopropoxy)- 2,2-dimethylpropyl)- carbamate | 569 |
| 27-2 | 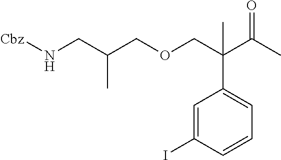 [SMILES: CC(=O)C(C)(COCC(C)CNC(=O)OCc1ccccc1)c1cccc(I)c1] | benzyl (3-(2-(3-iodo- phenyl)-2-methyl-3- oxobutoxy)-2-methyl- propyl)carbamate | 510 |
| 27-3 | 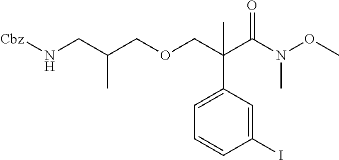 [SMILES: CON(C)C(=O)C(C)(COCC(C)CNC(=O)OCc1ccccc1)c1cccc(I)c1] | benzyl (3-(2-(3-iodo- phenyl)-3-(methoxy- (methyl)amino)-2- methyl-3-oxopropoxy)- 2-methylpropyl)- carbamate | 555 |
| 27-4 | 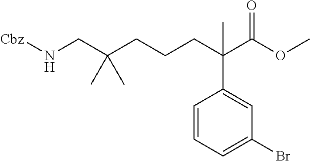 [SMILES: COC(=O)C(C)(CCCC(C)(C)CNC(=O)OCc1ccccc1)c1cccc(Br)c1] | methyl 7-(((benzyl- oxy)carbonyl)amino)- 2-(3-bromophenyl)- 2,6,6-trimethylhep- tanoate | 490, 492 |
| 27-5 | 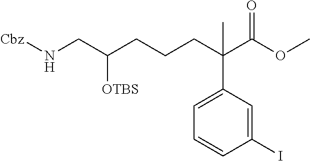 [SMILES: COC(=O)C(C)(CCCC(CNC(=O)OCc1ccccc1)O[Si](C)(C)C(C)(C)C)c1cccc(I)c1] | methyl 7-(((benzyloxy)- carbonyl)amino)-6- ((tert-butyldimethylsil- yl)oxy)-2-(3-iodophen- yl)-2-methylheptanoate | 640 |

### Intermediate 27A: 3-(3-Amino-2,2-dimethylpropoxy)-2-(3-iodophenyl)-N-methoxy-dimethylpropanamide

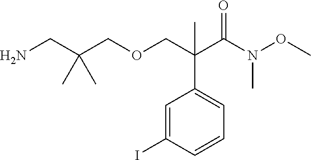 [SMILES: CON(C)C(=O)C(C)(COCC(C)(C)CN)c1cccc(I)c1]

To a stirred solution of 3-(3-azido-2,2-dimethylpropoxy)-2-(3-iodophenyl)-N-methoxy-N,2-dimethylpropanamide (Intermediate 11A-1, 4 g, 43.17 mmol) in tetrahydrofuran (30 mL) and water (3 mL) was added triphenylphosphine (2.5 g, 9.57 mmol). The mixture was stirred at 65° C. for 16 hours, cooled to room temperature and quenched with 50 mL of water. The solution was extracted with ethyl acetate (3×80 mL). The combined organic layers were dried over sodium sulfate and concentrated. The residue was washed with petroleum ether: ethyl acetate (4:1) and filtered. The filtrate was concentrated to give crude title compound (4.4 g, crude) as light-yellow liquid. MS-ESI: 435.0 m/z [M+H]⁺

The following intermediates were prepared utilizing the procedures described for Intermediate 27A.

| Inter. No. | Structure | Name | MS m/z [M + H]⁺ |
|---|---|---|---|
| 27A-1 | 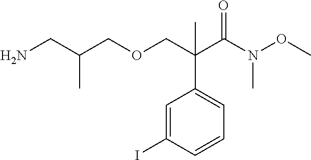 [SMILES: CON(C)C(=O)C(C)(COCC(C)CN)c1cccc(I)c1] | 3-(3-amino-2-methylpropoxy)- 2-(3-iodophenyl)-N-methoxy- N,2-dimethylpropanamide | 421 |
| 27A-2 | 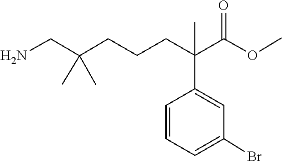 [SMILES: COC(=O)C(C)(CCCC(C)(C)CN)c1cccc(Br)c1] | methyl 7-amino-2-(3-bromo- phenyl)-2,6,6-trimethyl- heptanoate | 356, 358 |
| 27A-3 | 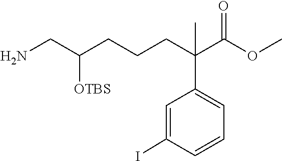 [SMILES: COC(=O)C(C)(CCCC(CN)O[Si](C)(C)C(C)(C)C)c1cccc(I)c1] | methyl 7-amino-6-((tert-butyl- dimethylsilyl)oxy)-2-(3-iodo- phenyl)-2-methylheptanoate | 506 |

### Intermediate 27B: 4-(3-Amino-2,2-dimethylpropoxy)-3-(3-iodophenyl)-3-methylbutan-2-one

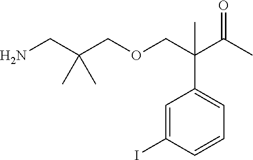 [SMILES: CC(=O)C(C)(COCC(C)(C)CN)c1cccc(I)c1]

To a stirred solution of 3-(3-amino-2,2-dimethylpropoxy)-2-(3-iodophenyl)-N-methoxy-N,2-dimethylpropanamide (Intermediate 27A, 4.5 g, 10.37 mmol) in tetrahydrofuran (60 mL) was added methyl magnesium bromide (35 mL, 103.69 mmol). The mixture was stirred at room temperature for 16 hours, quenched with 100 mL of the saturated ammonium chloride, and extracted with ethyl acetate (3×100 mL). The combined organic layers were dried over sodium sulfate and concentrated to give the crude title compound (4.2 g, crude) as a light-yellow liquid, which was used for next step without further purification. MS-ESI: 390.0 m/z [M+H]⁺

The following intermediates were prepared utilizing the procedures described for Intermediate 27B.

| Inter. No. | Structure | Name | MS m/z [M + H]⁺ |
|---|---|---|---|
| 27B-1 | 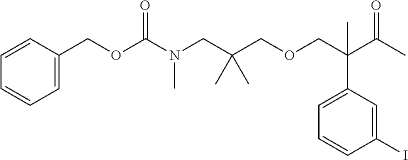 [SMILES: CC(=O)C(C)(COCC(C)(C)CN(C)C(=O)OCc1ccccc1)c1cccc(I)c1] | benzyl (3-(2-(3-iodo- phenyl)-2-methyl-3- oxobutoxy)-2,2- dimethylpropyl) (methyl)carbamate | 538 |
| 27B-2 | 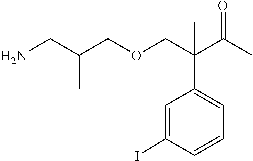 [SMILES: CC(=O)C(C)(COCC(C)CN)c1cccc(I)c1] | 4-(3-amino-2-methyl- propoxy)-3-(3-iodo- phenyl)-3-methyl- butan-2-one | 376 |
| 27B-3 | 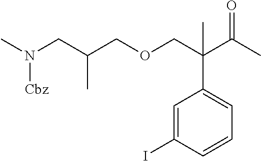 [SMILES: CC(=O)C(C)(COCC(C)CN(C)C(=O)OCc1ccccc1)c1cccc(I)c1] | benzyl (3-(2-(3-iodo- phenyl)-2-methyl-3- oxobutoxy)-2-meth- ylpropyl)(methyl) carbamate | 524 |

### Intermediate 28: Benzyl (3-(2-(3-iodophenyl)-3-(methoxy(methyl)amino)-2-methyl-3-oxopropoxy)-2,2-dimethylpropyl)(methyl)carbamate

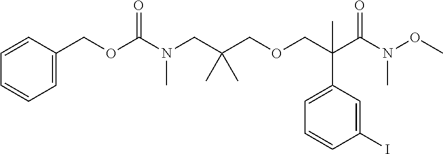 [SMILES: CON(C)C(=O)C(C)(COCC(C)(C)CN(C)C(=O)OCc1ccccc1)c1cccc(I)c1]

To a stirred and cooled (0° C.) solution of benzyl (3-(2-(3-iodophenyl)-3-(methoxy(methyl)amino)-2-methyl-3-oxopropoxy)-2,2-dimethylpropyl)carbamate (Intermediate 27-1, 4 g, 7.04 mmol) in tetrahydrofuran (30 mL) was added sodium hydride (422 mg, 10.56 mmol). The mixture was stirred at 0° C. for 1 hour, then treated with iodomethane (1.5 g, 10.56 mmol), and stirred for additional 16 hours at room temperature. The reaction was quenched with 50 mL of water and extracted with ethyl acetate (3×50 mL). The combined organic layers were dried over sodium sulfate and concentrated. The residue was purified by silica gel column chromatography (eluting with petroleum ether/ethyl acetate=3/1) to give the title compound (3.67 g, 90%) as light-yellow liquid. MS (ESI): 583 m/z [M+H]⁺.

The following intermediates were prepared utilizing the procedures described for Intermediate 28.

| Inter. No. | Structure | Name | MS m/z [M + H]⁺ |
|---|---|---|---|
| 28-1 | 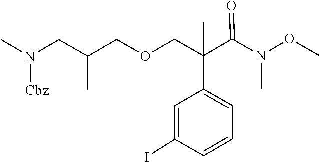 | benzyl (3-(2-(3-iodophenyl)-3- (methoxy(methyl)amino)-2- methyl-3-oxopropoxy)-2-meth- ylpropyl)(methyl)carbamate | 569 |
| 28-2 | 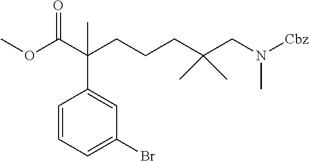 [SMILES: COC(=O)C(C)(CCCC(C)(C)CN(C)C(=O)OCc1ccccc1)c1cccc(Br)c1] | methyl 7-(((benzyloxy)carbon- yl)(methyl)amino)-2-(3- bromophenyl)-2,6,6-trimeth- ylheptanoate | 526, 528 [M + Na]⁺ |
| 28-3 | 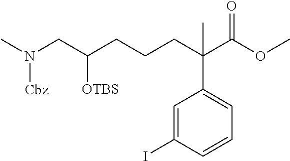 | methyl 7-(((benzyloxy)carbon- yl)(methyl)amino)-6-((tert- butyldimethylsilyl)oxy)-2-(3- iodophenyl)-2-methylheptanoate | 654 |

### Intermediate 29: 2-Fluoro-5-((6-fluoro-4-(4-hydroxybutyl)-1-tosyl-1H-indol-5-yl)oxy)benzonitrile

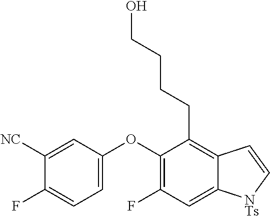 [SMILES: Cc1ccc(S(=O)(=O)n2ccc3c(CCCCO)c(Oc4ccc(F)c(C#N)c4)c(F)cc32)cc1]

To a stirred solution of 2-fluoro-5-((6-fluoro-4-(4-hydroxybut-1-yn-1-yl)-1-tosyl-1H-indol-5-yl)oxy)benzonitrile (Intermediate 29A, 4 g, 9.12 mmol) in ethyl acetate (50 mL) was added palladium on carbon (10%, 50% wet, 400 mg). The mixture was stirred at room temperature under hydrogen balloon for 16 hours, then filtered through a pad of Celite. The filtrate was concentrated, The residue was purified by silica gel column chromatography (eluting with petroleum ether/ethyl acetate=2/1) to give the title compound (2 g, 50%) as a colorless liquid. MS (ESI): 497 m/z [M+H]⁺, retention time: 2.11 minutes, purity: 98% (214 nm) (LC-MS method 8). ¹H NMR (400 MHz, CD₃OD) δ 7.86 (d, J=8.4 Hz, 2H), 7.77 (t, J=8.8 Hz, 1H), 7.72 (d, J=3.8 Hz, 1H), 7.35 (d, J=8.0 Hz, 2H), 7.31-7.22 (m, 1H), 7.22-7.12 (m, 2H), 6.85 (dt, J=9.5, 4.7 Hz, 1H), 3.48 (t, J=6.3 Hz, 2H), 2.80 (t, J=7.6 Hz, 2H), 2.36 (s, 3H), 1.71-1.42 (m, 4H) ppm.

The following intermediate was prepared utilizing the procedures described for Intermediate 29.

| Inter. No. | Structure | Name | MS m/z [M + H]⁺ |
|---|---|---|---|
| 29-1 | 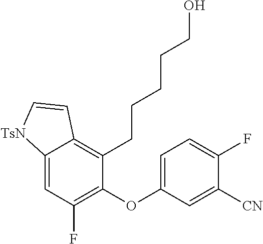 [SMILES: Cc1ccc(S(=O)(=O)n2ccc3c(CCCCCO)c(Oc4ccc(F)c(C#N)c4)c(F)cc32)cc1] | 2-fluoro-5-((6-fluoro-4-(5- hydroxypentyl)-1-tosyl-1H- indol-5-yl)oxy)benzonitrile | 511 |

### Intermediate 29A: 2-Fluoro-5-((6-fluoro-4-(4-hydroxybut-1-yn-1-yl)-1-tosyl-1H-indol-5-yl)oxy)benzonitrile

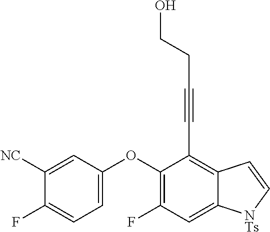 [SMILES: Cc1ccc(S(=O)(=O)n2ccc3c(C#CCCO)c(Oc4ccc(F)c(C#N)c4)c(F)cc32)cc1]

To a stirred and degassed solution of 5-((4-bromo-6-fluoro-1-tosyl-1H-indol-5-yl)oxy)-2-fluorobenzonitrile (Intermediate 3, 4.0 g, 8.0 mmol) in N,N-dimethylformamide (50 mL) was added bis(triphenylphosphine)palladium(II) dichloride (560 mg, 0.80 mmol), copper (I) iodide (304 mg, 1.60 mmol), tri(tert-butyl)phosphine (648 mg, 3.2 mmol), diisopropylamine (4.0 g, 40.0 mmol) and but-3-yn-1-ol (1.4 g, 20.0 mmol). The mixture was stirred at 100° C. overnight, cooled to room temperature and quenched with water (100 mL). The solution was extracted with ethyl acetate (3×80 mL). The combined organic layers were washed with brine, dried over sodium sulfate, and concentrated. The residue was purified by flash column (petroleum ether/ethyl acetate=3/1) to give the title compound (2.4 g, 61%) as a yellow solid. ¹H NMR (400 MHz, CDCl₃) δ 7.89-7.85 (m, 1H), 7.82 (d, J=8.0 Hz, 2H), 7.68 (d, J=3.6 Hz, 1H), 7.32 (d, J=8.0 Hz, 2H), 7.16-7.12 (m, 2H), 7.03-6.99 (m, 1H), 6.70 (d, J=3.6 Hz, 1H), 3.65-3.57 (m, 2H), 2.40 (s, 3H), 2.26-2.18 (m, 2H) ppm.

The following intermediate was prepared utilizing the procedures described for Intermediate 29A.

| Inter. No. | Structure | Name | MS m/z [M + H]⁺ |
|---|---|---|---|
| 29A-1 | 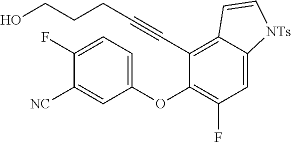 [SMILES: Cc1ccc(S(=O)(=O)n2ccc3c(C#CCCCO)c(Oc4ccc(F)c(C#N)c4)c(F)cc32)cc1] | 2-fluoro-5-((6-fluoro-4-(5- hydroxypent-1-yn-1-yl)-1- tosyl-1H-indol-5-yl)oxy)- benzonitrile | MS: 507.1 m/z [M+H]+. ¹H NMR (400 MHz, CDCl₃) δ 7.85-7.75 (m, 3H), 7.62 (d, J = 3.6 Hz, 1H), 7.30 (d, J = 8.0 Hz, 2H), 7.14 (d, J = 8.0 Hz, 2H), 7.01 (m, 1H), 6.75 (d, J = 3.6 Hz, 1H), 3.67-3.62 (m, 2H), 2.52-2.47 (m, 2H), 2.39 (s, 3H), 1.74-1.70 (m, 2H) ppm |

### Intermediate 30: Tert-Butyl((6-iodo-2,2-dimethylhexyl)oxy)dimethylsilane

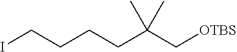 [SMILES: CC(C)(CCCCI)CO[Si](C)(C)C(C)(C)C]

To a stirred solution of tert-butyl-(6-chloro-2,2-dimethyl-hexoxy)-dimethyl-silane (Intermediate 30C, 27.00 g, 96.8 mmol) in acetone (300 mL) was added sodium iodide (43.50 g, 290 mmol) at room temperature. The mixture was stirred at 60° C. for 16 hours. The solvent was evaporated. The residue was dissolved in ethyl acetate (500 mL), washed with water (2×300 mL), dried over anhydrous sodium sulfate and concentrated. The crude residue was purified by flash chromatography (330 g silica gel column, petroleum ether) to give the title compound as a colorless oil (32.00 g, 89.3%). ¹H NMR (400 MHz, CDCl₃) 3.25-3.13 (m, 4H), 1.80-1.68 (m, 2H), 1.39-1.27 (m, 2H), 1.23-1.16 (m, 2H), 0.87 (s, 9H), 0.80 (s, 6H), −0.01 (s, 6H).

The following intermediates were prepared based on the procedures described for Intermediate 30.

| Inter. No. | Structure | Name | MS m/z [M + H]⁺ |
|---|---|---|---|
| 30-1 | 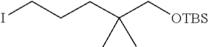 [SMILES: CC(C)(CCCI)CO[Si](C)(C)C(C)(C)C] | tert-butyl((5-iodo-2,2-dimethyl- pentyl)oxy)dimethylsilane | 357 |
| 30-2 | 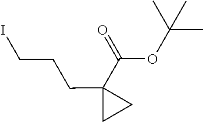 | tert-butyl 1-(3-iodopropyl)- cyclopropane-1-carboxylate | 311 |
| 30-3 | 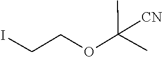 | 2-(2-iodoethoxy)-2-methyl- propanenitrile | ¹H NMR (400 MHz, CDCl₃) δ 3.85 (t, J = 6.7 Hz, 2H), 3.26 (t, J = 6.7 Hz, 2H), 1.60 (s, 6H). |
| 30-4 | 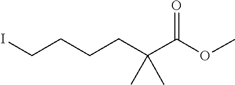 | methyl 6-iodo-2,2-dimethyl- hexanoate | 285 |
| 30-5 | 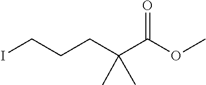 | methyl 5-iodo-2,2-dimethyl- pentanoate | ¹H NMR (400 MHz, CDCl₃) δ 3.65 (s, 3H), 3.13 (t, J = 6.8 Hz, 2H), 1.78-1.71 (m, 2H), 1.61- 1.57 (m, 2H), 1.16 (s, 6H) ppm. |
| 30-6 | 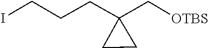 [SMILES: CC(C)(C)[Si](C)(C)OCC1(CCCI)CC1] | tert-butyl((1-(3-iodopropyl)- cyclopropyl)methoxy)dimethyl silane | 355 |

### Intermediate 30A: Methyl 6-chloro-2,2-dimethyl-hexanoate

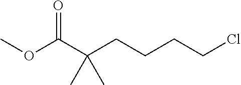

To a stirred and cooled (−78° C.) solution of methyl 2-methylpropanoate (10.30 g, 101 mmol) in tetrahydrofuran (200 mL) was added lithium diisopropylamide (50.4 mL, 101 mmol) drop wise. After stirring at −78° C. for 1 hour, 1-chloro-4-iodo-butane (20.00 g, 91.5 mmol) was added. The mixture was stirred at −78° C. for 1 hour and then allowed to warm to room temperature and stirred for an additional 16 hours. The reaction was quenched with 1M hydrochloric acid (200 mL) and extracted with ethyl acetate (2×200 mL). The combined organic phases were washed with water, brine, dried over sodium sulfate and concentrated. The crude residue was purified by flash silica gel chromatography (50% ethyl acetate in petroleum ether) to give the title compound as a light-yellow oil (16.00 g, 82.9%). MS (ESI): 193 m/z [M+H]⁺.

The following intermediates were prepared utilizing the procedures described for Intermediate 30A.

| Inter. No. | Structure | Name | MS m/z [M + H]⁺ |
|---|---|---|---|
| 30A-1 | 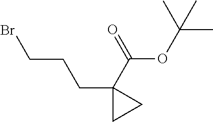 | tert-butyl 1-(3-bromo- propyl)cyclopropane- 1-carboxylate | 285, 287 [M + Na]⁺ |
| 30A-2 | 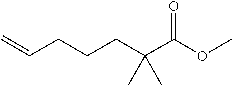 | methyl 2,2-dimethyl- hept-6-enoate | ¹H NMR (400 MHz, CDCl₃) δ 5.87- 5.70 (m,1H), 5.03-4.89 (m, 2H), 3.66 (s, 3H), 2.08-1.99 (m, 2H), 1.57-1.48 (m, 2H), 1.35-1.23 (m, 2H), 1.17 (s, 6H). |
| 30A-3 | 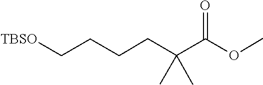 [SMILES: COC(=O)C(C)(C)CCCCO[Si](C)(C)C(C)(C)C] | methyl 6-((tert-butyl- dimethylsilyl)oxy)- 2,2-dimethylhexanoate | ¹H NMR (400 MHz, CDCl₃) δ 3.61 (s, 3H), 3.55 (t, J = 6.4 Hz, 2H), 1.50-1.42 (m, 4H), 1.22-1.19 (m, 2H), 1.12 (s, 6H), 0.82 (s, 9H), 0.00 (s, 6H) ppm. |
| 30A-4 | 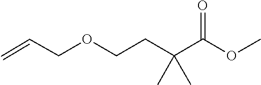 | methyl 4-(allyloxy)- 2,2-dimethylbutanoate | 1HNMR (400 MHz, CDCl₃) δ 5.93- 5.83 (m, 1H), 5.27-5.13 (m, 2H), 3.86-3.84 (m, 2H), 3.73 (s, 3H), 3.46 (t, J = 6.4 Hz, 2H), 1.86 (t, J = 6.4 Hz, 2H), 1.13 (s, 6H) ppm. |
| 30A-5 | 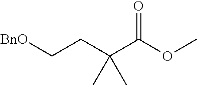 [SMILES: COC(=O)C(C)(C)CCOCc1ccccc1] | methyl 4-(benzyloxy)- 2,2-dimethylbutanoate | 237 |
| 30A-6 | 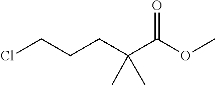 | methyl 5-chloro-2,2- dimethylpentanoate | ¹H NMR (400 MHz, CDCl₃) 3.66 (s, 3H), 3.49 (t, J = 6.4 Hz, 2H), 1.75- 1.60 (m, 4H), 1.18 (s, 6H) ppm |

### Intermediate 30B: 6-Chloro-2,2-dimethylhexan-1-ol

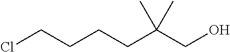

To a stirred and cooled (−78° C.) solution of methyl 6-chloro-2,2-dimethyl-hexanoate (Intermediate 30A, 24.00 g, 125 mmol) in tetrahydrofuran (200 mL) was added lithium aluminum hydride (7.09 g, 187 mmol) dropwise over 30 minutes. The mixture was stirred for 3 hours at −78° C., then quenched with 7 mL of water, warmed to 0° C., followed by 7 mL of 15% sodium hydroxide, and finally 21 mL of water. The mixture was stirred at room temperature for 15 minutes and then filtered. The filtrate was concentrated to give the title compound as an oil (18.00 g, 87.8%). H NMR (400 MHz, CDCl₃) δ 3.55 (t, J=6.7 Hz, 2H), 3.53 (s, 2H), 1.80-1.73 (m, 2H), 1.4-1.39 (m, 2H), 1.28-1.26 (m, 2H), 0.86 (s, 6H) ppm.

The following intermediates were prepared utilizing the procedures described for Intermediate 30B.

| Inter. No. | Structure | Name | MS m/z [M + H]⁺ |
|---|---|---|---|
| 30B-1 | 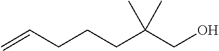 | 2,2-dimethylhept- 6-en-1-ol | ¹H NMR (400 MHz, CDCl₃) δ 5.87-5.75 (m, 1H), 5.05-4.91 (m, 2H), 3.34-3.30 (m, 2H), 2.07-2.01 (m, 2H), 1.36-1.29 (m, 2H), 1.27- |
| | | | 1.21 (m, 2H), 0.87 (s, 6H) ppm. |
| 30B-2 | 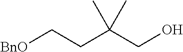 | 4-(benzyloxy)-2,2- dimethylbutan-1-ol | 209 |

### Intermediate 30C: Tert-Butyl-(6-chloro-2,2-dimethyl-hexoxy)-dimethyl-silane

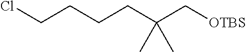 [SMILES: CC(C)(CCCCCl)CO[Si](C)(C)C(C)(C)C]

To a stirred solution of 6-chloro-2,2-dimethyl-hexan-1-ol (Intermediate 30B, 18.00 g, 109 mmol) in dichloromethane (300 mL) was added tert-butyldimethylsilyl chloride (19.80 g, 131 mmol) and imidazole (14.9 g, 219 mmol). The mixture was stirred at room temperature for 2 hours, then diluted with dichloromethane (200 mL). The solution was washed with water (2×100 mL), brine, dried over anhydrous sodium sulfate and concentrated. The crude residue was purified by flash chromatography (120 g silica gel column, petroleum ether) to give the title compound as an oil (27.00 g, 89%). ¹H NMR (400 MHz, CDCl₃) δ 3.52 (t, J=6.7 Hz, 2H), 3.21 (s, 2H), 1.76-1.68 (m, 2H), 1.35 (dt, J=10.7, 7.9 Hz, 2H), 1.23-1.18 (m, 2H), 0.87 (s, 9H), 0.80 (d, J=5.8 Hz, 6H), −0.01 (s, 6H) ppm.

The following intermediates were prepared utilizing the procedures described for Intermediate 30C.

| Inter. No. | Structure | Name | MS m/z [M + H]⁺ |
|---|---|---|---|
| 30C-1 | 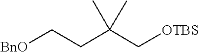 [SMILES: CC(C)(CCOCc1ccccc1)CO[Si](C)(C)C(C)(C)C] | (4-(benzyloxy)-2,2- dimethylbutoxy)(tert- butyl)dimethylsilane | ¹H NMR (400 MHz, CDCl₃) δ 7.33 (d, J = 4.4 Hz, 4H), 7.28-7.26 (m, 1H), 4.49 (s, 2H), 3.53 (t, J = 7.6 Hz, 2H), 3.23 (s, 2H), 1.61 (t, J = |
| | | | 7.6 Hz, 2H), 0.88 (s, 9H), 0.86 (s, |
| | | | 6H), 0.00 (s, 6H) ppm. |
| 30C-2 | 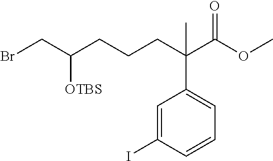 [SMILES: COC(=O)C(C)(CCCC(CBr)O[Si](C)(C)C(C)(C)C)c1cccc(I)c1] | methyl 7-bromo-6- ((tert-butyldimethyl- silyl)oxy)-2-(3-iodo- phenyl)-2-methyl- heptanoate | 569, 571 |

### Intermediate 31: Methyl 7-(((benzyloxy)carbonyl)(methyl)amino)-2-(3-bromo-2-fluorophenyl)-6,6-dimethylheptanoate

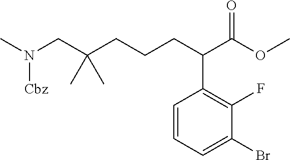

To a stirred and cooled (0° C.) solution of methyl 2-(3-bromo-2-fluorophenyl)-6,6-dimethyl-7-(methylamino)heptanoate (Intermediate 31C, 3.4 g, 9.11 mmol) and triethylamine (2.8 g, 27.3 mmol) in dichloromethane (50 mL) was added benzyl chloroformate (1.86 g, 10.93 mmol). The mixture was stirred at room temperature for 60 minutes, then diluted with dichloromethane/brine (3:1, 20 mL). The separated organic layer, combined with two additional dichloromethane extracts (2×20 mL), was dried over sodium sulfate, and concentrated. The residue was purified by silica gel column chromatography (eluting with petroleum ether/ethyl acetate=5/1) to give the title compound (3.3 g, 71%) as a yellow oil. MS (ESI): 530 m/z [M+Na]⁺, retention time: 2.35 minutes, purity: 86% (214 nm) (LC-MS method 7).

The following intermediates were prepared utilizing the procedures described for Intermediate 31.

| Inter. No. | Structure | Name | MS m/z [M + H]⁺ |
|---|---|---|---|
| 31-1 | 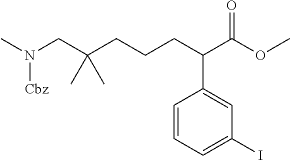 | methyl 7-(((benzyloxy)carbon- yl)(methyl)amino)-2-(3-iodop- henyl)-6,6-dimethylheptanoate | 560 [M + Na]⁺ |
| 31-2 | 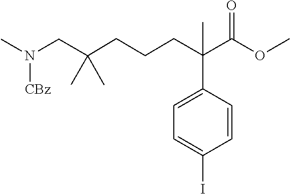 | methyl 7-(((benzyloxy)carbon- yl)(methyl)amino)-2-(4-iodo- phenyl)-2,6,6-trimethyl- heptanoate | 574 [M + Na]⁺ |
| 31-3 | 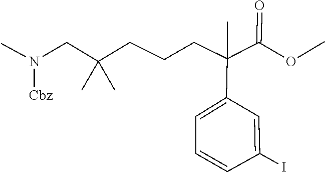 | methyl 7-(((benzyloxy)carbon- yl)(methyl)amino)-2-(3-iodo- phenyl)-2,6,6-trimethyl- heptanoate | 574 [M + Na]⁺ |
| 31-4 | 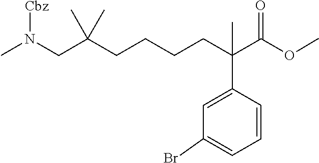 | methyl 8-(((benzyloxy)carbon- yl)(methyl)amino)-2-(3- bromophenyl)-2,7,7-trimethyl- octanoate | 540, 542 [M + Na]⁺ |
| 31-5 | 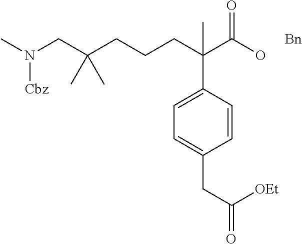 [SMILES: CCOC(=O)Cc1ccc(C(C)(CCCC(C)(C)CN(C)C(=O)OCc2ccccc2)C(=O)O)cc1 ; "Bn"] | benzyl 7-(((benzyloxy)carbon- yl)(methyl)amino)-2-(4-(2- ethoxy-2-oxoethyl)phenyl)- 2,6,6-trimethylheptanoate | 610 [M + Na]⁺ |
| 31-6 | 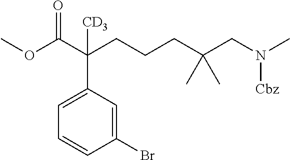 | methyl 7-(((benzyloxy)carbon- yl)(methyl)amino)-2-(3- bromophenyl)-6,6-dimethyl-2- (methyl-d3)heptanoate | 529, 531 [M + Na]⁺ |
| 31-7 | 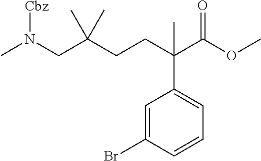 | methyl 6-(((benzyloxy)carbon- yl)(methyl)amino)-2-(3- bromophenyl)-2,5,5-trimethyl- hexanoate | 490, 492 |
| 31-8 | 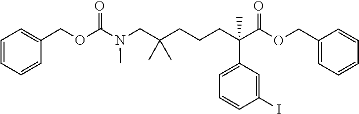 [SMILES: CN(CC(C)(C)CCC[C@@](C)(C(=O)OCc1ccccc1)c1cccc(I)c1)C(=O)OCc1ccccc1] | benzyl (R)-7-((benzyloxy)- carbonyl)(methyl)amino)-2- (3-iodophenyl)-2,6,6-trimeth- ylheptanoate | 628 |
| 31-9 | 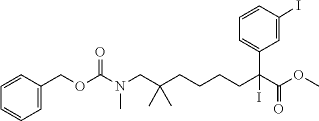 [SMILES: COC(=O)C(I)(CCCCC(C)(C)CN(C)C(=O)OCc1ccccc1)c1cccc(I)c1] | methyl (R)-8-(((benzyloxy)- carbonyl)(methyl)amino)-2- (3-iodophenyl)-2,7,7-trimeth- yloctanoate | 566 |

### Intermediate 31A: Methyl 2-(3-bromo-2-fluorophenyl)-7-hydroxy-6,6-dimethylheptanoate

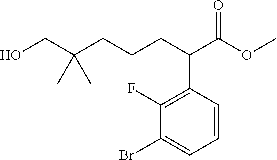 [SMILES: COC(=O)C(CCCC(C)(C)CO)c1cccc(Br)c1F]

To a solution of methyl 2-(3-bromo-2-fluorophenyl)-7-((tert-butyldimethylsilyl)oxy)-6,6-dimethylheptanoate (Intermediate 19A-5, 6.2 g, 13 mmol) in tetrahydrofuran (50 mL) was added tetrabutylammonium fluoride (1M in tetrahydrofuran, 13 mL, 26 mmol). The resulting mixtures were stirred at room temperature for 16 hours and concentrated. The residue was quenched with brine (30 mL) and extracted with ethyl acetate (3×100 mL). The combined organic phases were dried over sodium sulfate and concentrated. The crude product was purified by silica gel column chromatography (eluting with petroleum ether/ethyl acetate=4/1) to give the title compound (4.3 g, 93%) as colorless oil. MS (ESI): 361 m/z [M+H]⁺, retention time: 2.10 minutes, purity: 90% (214 nm) (LC-MS method 5).

The following intermediates were prepared utilizing the procedures described for Intermediate 31A.

| Inter. No. | Structure | Name | MS m/z [M + H]⁺ |
|---|---|---|---|
| 31A-1 | 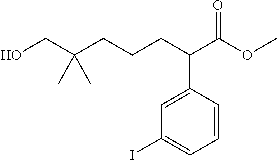 [SMILES: COC(=O)C(CCCC(C)(C)CO)c1cccc(I)c1] | methyl 7-hydroxy-2-(3- iodophenyl)-6,6- dimethylheptanoate | 391 |
| 31A-2 | 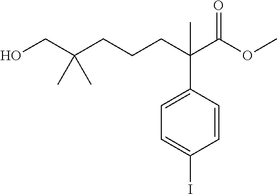 [SMILES: COC(=O)C(C)(CCCC(C)(C)CO)c1ccc(I)cc1] | methyl 7-hydroxy-2- (4-iodophenyl)-2,6,6- trimethylheptanoate | 427 [M + Na]⁺ |
| 31A-3 | 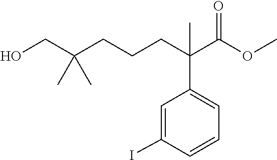 [SMILES: COC(=O)C(C)(CCCC(C)(C)CO)c1cccc(I)c1] | methyl 7-hydroxy-2- (3-iodophenyl)-2,6,6- trimethylheptanoate | MS: 427 [M + Na]⁺; ¹H NMR (400 MHz, CDCl₃) δ 7.62 (s, 1H), 7.57 (d, J = 8.0 Hz, 1H), 7.26-7.25 (m, 1H), 7.05 (t, J = 8.0 Hz, 1H), 3.66 (s, 3H), 3.20 (s, 2H), 2.04-1.95 (m, 1H), 1.85-1.77 (m, 1H), 1.52 (s, 3H), 1.28-1.10 (m, 4H), 0.83 (s, 6H) ppm |
| 31A-4 | 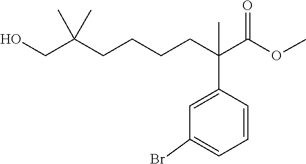 [SMILES: COC(=O)C(C)(CCCCC(C)(C)CO)c1cccc(Br)c1] | methyl 2-(3-bromophen- yl)-8-hydroxy-2,7,7- trimethyloctanoate | ¹H NMR (400 MHz, CDCl₃) δ 7.44-7.43(m, 1H), 7.38-7.36 (m, 1H), 7.26-7.25 (m, 1H), 7.21-7.18 (m, 1H), 3.66 (s, 3H), 3.30 (s, 2H), 2.03-1.84 (m, 2H), 1.51 (s, 3H), 1.26- 1.18 (m, 6H), 0.85 (s, 6H) ppm. |
| 31A-5 | 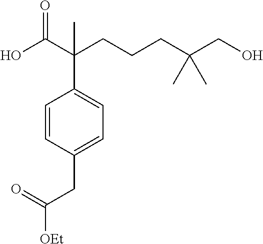 [SMILES: CCOC(=O)Cc1ccc(C(C)(CCCC(C)(C)CO)C(=O)O)cc1] | 2-(4-(2-ethoxy-2-oxo- ethyl)phenyl)-7- hydroxy-2,6,6-trimeth- ylheptanoic acid | 351 |
| 31A-6 | 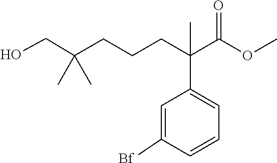 | methyl 2-(3-bromophen- yl)-7-hydroxy-2,6,6- trimethylheptanoate | 379, 381 [M + Na]⁺ |
| 31A-7 | 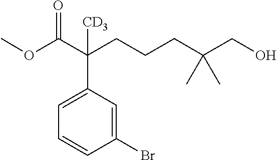 [SMILES: [2H]C([2H])([2H])C(CCCC(C)(C)CO)(C(=O)OC)c1cccc(Br)c1] | methyl 2-(3-bromophen- yl)-7-hydroxy-6,6- dimethyl-2-(methyl- d3)heptanoate | 360, 362 |
| 31A-8 | 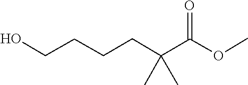 | methyl 6-hydroxy-2,2- dimethylhexanoate | ¹H NMR (400 MHz, CDCl₃) δ 3.56 (s, 3H), 3.54 (t, J = 6.4 Hz, 2H), 1.62 (br s, 1H), 1.48- 1.41 (m, 4H), 1.23-1.15 (m, 2H), 1.07 (s, 6H) ppm. |
| 31A-9 | 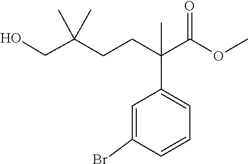 [SMILES: COC(=O)C(C)(CCC(C)(C)CO)c1cccc(Br)c1] | methyl 2-(3-bromophen- yl)-6-hydroxy-2,5,5- trimethylhexanoate | 343, 345 |
| 31A-10 | 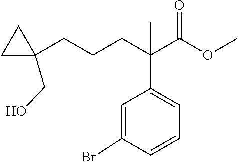 [SMILES: COC(=O)C(C)(CCCC1(CO)CC1)c1cccc(Br)c1] | methyl 2-(3-bromophen- yl)-5-(1-(hydroxymeth- yl)cyclopropyl)-2-meth- ylpentanoate | 355 |
| 31A-11 | 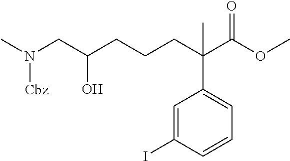 | methyl 7-(((benzyloxy)- carbonyl)(methyl)- amino)-6-hydroxy-2-(3- iodophenyl)-2-methyl- heptanoate | 540 |

### Intermediate 31B: Methyl 2-(3-bromo-2-fluorophenyl)-6,6-dimethyl-7-oxoheptanoate

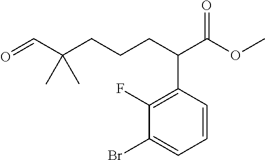 [SMILES: COC(=O)C(CCCC(C)(C)C=O)c1cccc(Br)c1F]

To a stirred and cooled (0° C.) solution of methyl 2-(3-bromo-2-fluorophenyl)-7-hydroxy-6,6-dimethylheptanoate (Intermediate 31A, 4.3 g, 11.9 mmol) and 100 mesh silica (6.4 g) in dichloromethane (50 mL) was added pyridinium chlorochromate (6.4 g, 30 mmol). The mixture was stirred at room temperature for 3 hours and filtered through a pad of Celite. The filter cake was washed with dichloromethane (50 mL). The combined filtrate was concentrated. The residue was purified by silica gel column chromatography (eluting with petroleum ether/ethyl acetate=5/1) to give the title compound (3.3 g, 77%) as colorless oil. MS (ESI): 359, 361 m/z [M+H]⁺, retention time: 2.17 minutes, purity: 88% (214 nm) (LC-MS method 5). ¹H NMR (400 MHz, CHCl₃) δ 9.41 (s, 1H), 7.43-7.48 (m, 2H), 6.98-7.03 (m, 1H), 3.92 (t, J=7.6 Hz, 1H), 3.68 (s, 3H), 2.04-2.08 (m, 1H), 1.72-1.75 (m, 1H), 1.45-1.51 (m, 2H), 1.14-1.23 (m, 2H), 1.01 (s, 6H) ppm

The following intermediates were prepared utilizing the procedures described for Intermediate 31B.

| Inter. No. | Structure | Name | MS m/z [M + H]⁺ |
|---|---|---|---|
| 31B-1 | 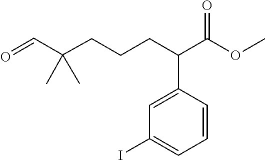 [SMILES: COC(=O)C(CCCC(C)(C)C=O)c1cccc(I)c1] | methyl 2-(3-iodo- phenyl)-6,6-dimeth- yl-7-oxoheptanoate | 389 |
| 31B-2 | 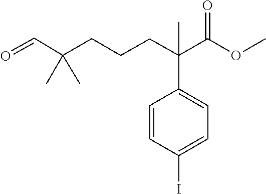 [SMILES: COC(=O)C(C)(CCCC(C)(C)C=O)c1ccc(I)cc1] | methyl 2-(4-iodo- phenyl)-2,6,6- trimethyl-7-oxo- heptanoate | 425 [M + Na]⁺ |
| 31B-3 | 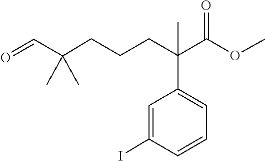 [SMILES: COC(=O)C(C)(CCCC(C)(C)C=O)c1cccc(I)c1] | methyl 2-(3-iodo- phenyl)-2,6,6- trimethyl-7-oxo- heptanoate | ¹H NMR (400 MHz, CDCl₃) δ 9.41 (s, 1H),7.61 (s, 1H), 7.57 (d, J = 8.0 Hz, 1H), 7.23 (d, J = 8.0 Hz, 1H),7.05 (t, J = 8.0 Hz, 1H), 3.65 (s, 3H), 2.03-1.95 (m, 1H), 1.84-1.77 (m, 1H), 1.50 (s, 3H), 1.47-1.43 (m, 2H), 1.13-1.04 (m, 2H), 1.01(d, J = 2.4 Hz, 6H) ppm |
| 31B-4 | 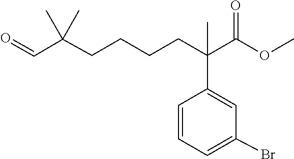 [SMILES: COC(=O)C(C)(CCCCC(C)(C)C=O)c1cccc(Br)c1] | methyl 2-(3-bromo- phenyl)-2,7,7- trimethyl-8-oxo- octanoate | 391, 393 [M + Na]⁺ |
| 31B-5 | 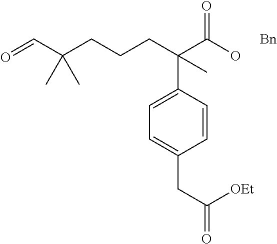 [SMILES: CCOC(=O)Cc1ccc(C(C)(CCCC(C)(C)C=O)C(=O)O)cc1 ; "Bn"] | benzyl 2-(4-(2- ethoxy-2-oxoethyl)- phenyl)-2,6,6- trimethyl-7-oxo- heptanoate | 439 |
| 31B-6 | 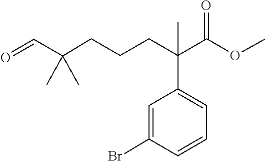 [SMILES: COC(=O)C(C)(CCCC(C)(C)C=O)c1cccc(Br)c1] | methyl 2-(3-bromo- phenyl)-2,6,6- trimethyl-7-oxo- heptanoate | 355, 357 |
| 31B-7 | 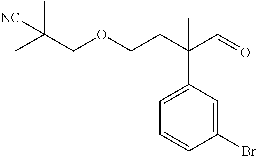 [SMILES: CC(C)(C#N)COCCC(C)(C=O)c1cccc(Br)c1] | 3-(3-(3-bromophen- yl)-3-methyl-4- oxobutoxy)-2,2- dimethylpropane- nitrile | 338, 340 |
| 31B-8 | 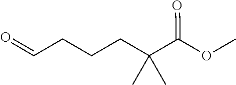 | methyl 2,2-dimeth- yl-6-oxohexanoate | ¹H NMR (400 MHz, CDCl₃) δ 9.66 (s, 1H), 3.57 (s, 3H), 2.34- 2.32 (m. 2H), 1.51-1.41 (m, 4H), 1.08 (m, 6H) ppm. |
| 31B-9 | 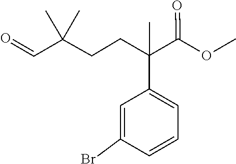 [SMILES: COC(=O)C(C)(CCC(C)(C)C=O)c1cccc(Br)c1] | methyl 2-(3-bromo- phenyl)-2,5,5- trimethyl-6-oxo- hexanoate | ¹H NMR (400 MHz, CDCl₃) δ 9.40 (s, 1H), 7.42-7.40 (m, 1H), 7.40-7.35 (m, 1H), 7.21-7.18 (m, 2H), 3.67 (s, 3H), 1.96-1.88 (m, 1H), 1.83-1.75 (m, 1H), 1.54 (s, 3H), 1.35-1.31 (m, 2H), 1.04 (s, 6H) ppm. |
| 31B-10 | 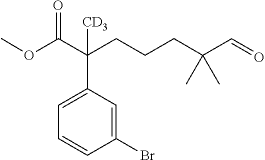 [SMILES: [2H]C([2H])([2H])C(CCCC(C)(C)C=O)(C(=O)OC)c1cccc(Br)c1] | methyl 2-(3-bromo- phenyl)-6,6-dimeth- yl-2-(methyl-d3)-7- oxoheptanoate | ¹H NMR (400 MHz, CDCl₃) δ 9.41 (s, 1H), 7.61 (s, 1H), 7.57(d, J = 8.0 Hz, 1H), 7.23 (d, J = 8.0 Hz, 1H), 7.05 (t, J = 8.0 Hz, 1H), 3.65 (s, 3H), 2.03-1.95 (m, 1H), 1.84-1.77 (m, 1H), 1.47-1.43 (m, 2H), 1.13-1.04 (m, 2H), 1.01 (d, J = 2.4 Hz, 6H) ppm. |
| 31B-11 | 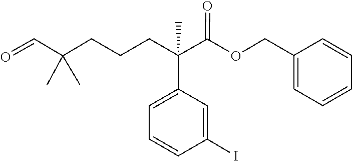 [SMILES: CC(C)(C=O)CCC[C@@](C)(C(=O)OCc1ccccc1)c1cccc(I)c1] | benzyl (R)-2-(3- iodophenyl)-2,6,6- trimethyl-7-oxo- heptanoate | ¹H NMR (400 MHz, DMSO-d₆) δ 9.34 (s, 1H), 7.63-7.60 (m, 1H), 7.56 (t, J = 5.6 Hz, 1H), 7.36- 7.27 (m, 4H), 7.25-7.22 (m, 2H), 7.13 (t, J = 7.6 Hz, 1H), 5.15-5.07 (m, 2H), 1.90-1.79 (m, 2H), 1.44 (s, 3H), 1.41-1.37 (m, 2H), 1.05- 0.95 (m, 2H), 0.89 (s, 6H) ppm. |
| 31B-12 | 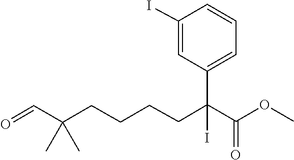 [SMILES: COC(=O)C(I)(CCCCC(C)(C)C=O)c1cccc(I)c1] | methyl (R)-2-(3- iodophenyl)-2,7,7- trimethyl-8-oxo- octanoate | 439 [M + Na]⁺ |

### Intermediate 31C: Methyl 2-(3-bromo-2-fluorophenyl)-6,6-dimethyl-7-(methylamino)heptanoate

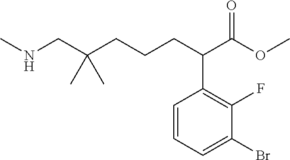

To a stirred solution of methyl 2-(3-bromo-2-fluorophenyl)-6,6-dimethyl-7-oxoheptanoate (Intermediate 31B, 3.3 g, 9.2 mmol) in methanol (30 mL) was added and a drop of acetic acid and methylamine (2M in tetrahydrofuran, 23 mmol, 46 mL). The mixture was stirred for 16 hours, then treated with sodium borohydride (1.05 g, 27.6 mmol), and stirred at room temperature for an additional 30 minutes. The reaction mixture was quenched with water (50 mL) and extracted with ethyl acetate (3×100 mL). The combined organic extracts were dried over sodium sulfate and concentrated to give the crude title compound (3.4 g, 94%) as a white solid, which was used for next step without further purification. MS (ESI): 374, 376 m/z [M+H]⁺, retention time: 1.70 minutes, purity: 86% (214 nm) (LC-MS method 5).

The following intermediates were prepared utilizing the procedures described for Intermediate 31C.

| Inter. No. | Structure | Name | MS m/z [M + H]⁺ |
|---|---|---|---|
| 31C-1 | 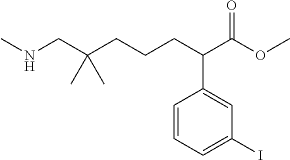 | methyl 2-(3-iodophenyl)- 6,6-dimethyl-7-(methyl- amino)heptanoate | 404 |
| 31C-2 | 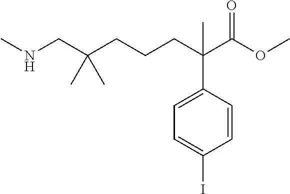 | methyl 2-(4-iodophenyl)- 2,6,6-trimethyl-7-(meth- ylamino)heptanoate | 418 |
| 31C-3 | 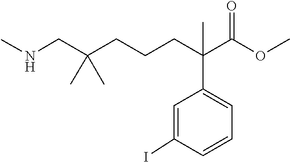 | methyl 2-(3-iodophenyl)- 2,6,6-trimethyl-7-(meth- ylamino)heptanoate | 418 |
| 31C-4 | 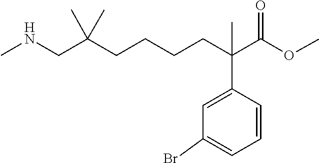 | methyl 2-(3-bromophen- yl)-2,7,7-trimethyl-8- (methylamino)octanoate | 384, 386 |
| 31C-5 | 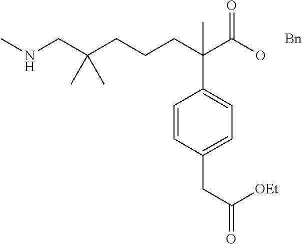 [SMILES: CCOC(=O)Cc1ccc(C(C)(CCCC(C)(C)CNC)C(=O)O)cc1 ; "Bn"] | benzyl 2-(4-(2-ethoxy-2- oxoethyl)phenyl)-2,6,6- trimethyl-7-(methyl- amino)heptanoate | 454 |
| 31C-6 | 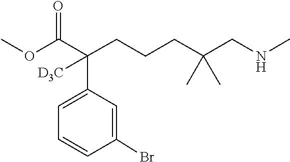 | methyl 2-(3-bromophen- yl)-6,6-dimethyl-2- (methyl-d3)-7-(methyl- amino)heptanoate | 373, 375 |
| 31C-7 | 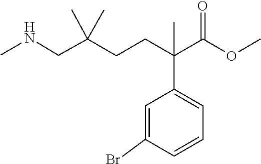 | methyl 2-(3-bromophen- yl)-2,5,5-trimethyl-6- (methylamino)hexanoate | ¹H NMR (400 MHz, CDCl₃) δ 7.45 (t, J = 1.6 Hz, 1H), 7.38 (dt, J = 7.6, 1.6 Hz, 1H), 7.25- 7.17 (m, 2H), 3.66 (s, 3H), 2.42 (s, 3H), 2.34-2.27 (m, 2H), 1.98-1.94 (m, 1H), 1.85- 1.79 (m, 1H), 1.52 (s, 3H), 1.11-1.06 (m, 2H), 0.87-0.86 (m, 6H) ppm. |
| 31C-8 | 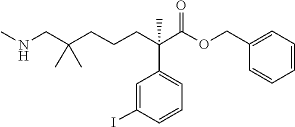 [SMILES: CNCC(C)(C)CCC[C@@](C)(C(=O)OCc1ccccc1)c1cccc(I)c1] | benzyl (R)-2-(3-iodo- phenyl)-2,6,6-trimeth- yl-7-(methylamino)- heptanoate | 494 |

### Intermediate 32: 6-Acetoxy-2-(3-bromo-2-fluorophenyl)-2-methylhexanoic acid

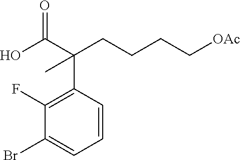 [SMILES: CC(=O)OCCCCC(C)(C(=O)O)c1cccc(Br)c1F]

A mixture of 2-(3-bromo-2-fluorophenyl)-6-hydroxy-2-methylhexanoic acid(Intermediate 32A, 1.4 g, 4.39 mmol) and p-toluenesulfonic acid (755 mg, 0.4 mmol) in acetic anhydride (8 mL) was stirred at room temperature for 1 hour, then treated with water (3 mL) and tetrahydrofuran (3 mL). The mixture was stirred at room temperature for 16 hours, diluted with water (50 mL), and extracted with ethyl acetate (3×50 mL). The combined organic layers were dried over sodium sulfate and concentrated. The residue was purified by flash chromatography (eluting with 0-20% ethyl acetate in petroleum ether) to give the title compound (1.3 g, 82%) as a colorless oil. MS (ESI): 383, 385 m/z [M+Na]⁺; retention time: 1.93 minutes, purity: 92% (214 nm) (LC-MS method 5).

The following intermediates were prepared based on the procedures described for Intermediate 32.

| Inter. | | | |
|---|---|---|---|
| No. | Structure | Name | MS m/z [M + H]⁺ |
| 32-1 | 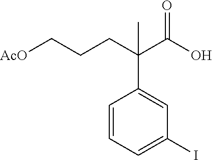 [SMILES: CC(=O)OCCCC(C)(C(=O)O)c1cccc(I)c1] | 5-acetoxy-2-(3-iodophenyl)-2- methylpentanoic acid | 377 |
| 32-2 | 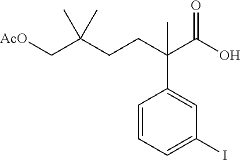 [SMILES: CC(=O)OCC(C)(C)CCC(C)(C(=O)O)c1cccc(I)c1] | 6-acetoxy-2-(3-iodophenyl)-2,5,5- trimethylhexanoic acid | 441 [M + Na]⁺ |
| 32-3 | 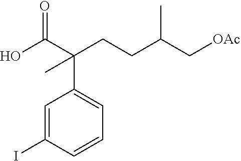 [SMILES: CC(=O)OCC(C)CCC(C)(C(=O)O)c1cccc(I)c1] | 6-acetoxy-2-(3-iodophenyl)-2,5- dimethylhexanoic acid | 427 [M + Na]⁺ |
| 32-4 | 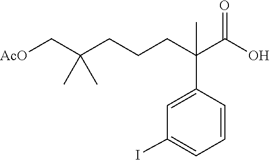 [SMILES: CC(=O)OCC(C)(C)CCCC(C)(C(=O)O)c1cccc(I)c1] | 7-acetoxy-2-(3-iodophenyl)-2,6,6- trimethylheptanoic acid | 455 [M + Na]⁺ |

### Intermediate 32A: 2-(3-Bromo-2-fluorophenyl)-6-hydroxy-2-methylhexanoic acid

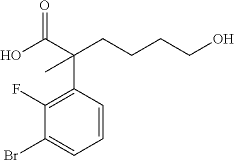 [SMILES: CC(CCCCO)(C(=O)O)c1cccc(Br)c1F]

To a stirred solution of methyl 2-(3-bromo-2-fluorophenyl)-6-((tert-butyldimethylsilyl)oxy)-2-methylhexanoate (Intermediate 19A-6, 3.4 g, 7.6 mmol) in 30 mL of ethanol was added a solution of sodium hydroxide (3.04 g, 76 mmol) in 5 mL of water. The reaction mixture was stirred at 60° C. for 16 hours and concentrate. The residue was dissolved in 50 mL of water, acidified to pH˜4 using 2 N of hydrochloric acid solution. The aqueous phase was extracted with ethyl acetate (3×20 mL). The combined organic phases were dried over sodium sulfate and concentrated. The crude product was purified by automated flash chromatography (eluting with 0-50% ethyl acetate in hexanes) to give the title compound (1.5 g, 62%) as a colorless oil. MS (ESI): 341, 343 m/z [M+Na]⁺; retention time: 1.76 minutes, purity: 92% (214 nm) (LC-MS method 5).

The following intermediates were prepared based on the procedures described for Intermediate 32A.

| Inter. | | | |
|---|---|---|---|
| No. | Structure | Name | MS m/z [M + H]⁺ |
| 32A-1 | 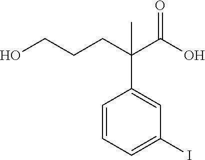 [SMILES: CC(CCCO)(C(=O)O)c1cccc(I)c1] | 5-hydroxy-2-(3-iodophenyl)-2- methylpentanoic acid | 335 |
| 32A-2 | 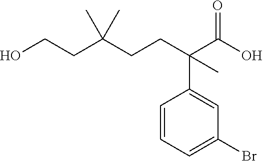 [SMILES: CC(C)(CCO)CCC(C)(C(=O)O)c1cccc(Br)c1] | 2-(3-bromophenyl)-7-hydroxy-2,5,5- trimethylheptanoic acid | 365, 367 [M + Na]⁺ |
| 32A-3 | 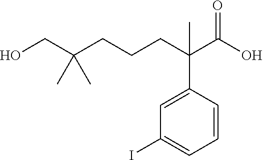 [SMILES: CC(C)(CO)CCCC(C)(C(=O)O)c1cccc(I)c1] | 7-hydroxy-2-(3-iodophenyl)-2,6,6- trimethylheptanoic acid | 391 |

### Intermediate 33: 6-Acetoxy-2-(3-bromo-2-fluorophenyl)hexanoic acid

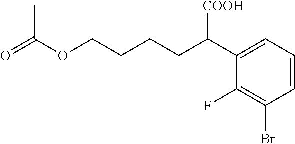 [SMILES: CC(=O)OCCCCC(C(=O)O)c1cccc(Br)c1F]

To a stirred solution of 2-(3-bromo-2-fluorophenyl)-6-hydroxyhexanoic acid (Intermediate 33B, 1.7 g, 0.00557 mol) in acetic anhydride (15 mL) was added p-toluenesulfonic acid (0.106 g, 0.000557 mol). The mixture was stirred for 1 hour, treated with tetrahydrofuran (15 mL) and water (30 mL), and stirred for additional 16 hours. The mixture was extracted with petroleum ether/ethyl acetate (1/1, 2×50 mL). The combined organic layers were washed with water and brine, dried over sodium sulfate, and concentrated to give the title compound (1.7 g, 79%) as a yellow oil. MS (ESI): 369, 371 m/z [M+Na]⁺, retention time: 1.89 minutes, purity: 89% (214 nm) (LC-MS method 14).

The following intermediate was prepared based on the procedures described for Intermediate 33.

| Inter. | | | |
|---|---|---|---|
| No. | Structure | Name | MS m/z [M + H]⁺ |
| 33-1 | 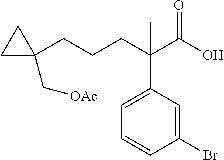 [SMILES: CC(=O)OCC1(CCCC(C)(C(=O)O)c2cccc(Br)c2)CC1] | 5-(1-(acetoxymethyl)cyclopropyl)-2- (3-bromophenyl)-2- methylpentanoic acid | 405, 407 [M + Na]⁺ |

### Intermediate 33A: 2-(3-Bromo-2-fluorophenyl)-6-((tert-butyldimethylsilyl)oxy)hexanenitrile

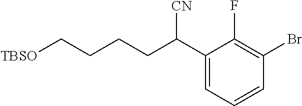 [SMILES: CC(C)(C)[Si](C)(C)OCCCCC(C#N)c1cccc(Br)c1F]

To a stirred and cooled (0° C.) solution of 2-(3-bromo-2-fluoro-phenyl)acetonitrile (2.14 g, 0.01 mol) in N,N-dimethylformamide (30 mL) was added sodium hydride (60% dispersion in mineral oil, 0.6 g, 0.015 mol). The mixture was stirred for 5 minutes, then treated with tert-butyl(4-iodobutoxy)dimethylsilane (3.14 g, 0.01 mol). The mixture was stirred at 0° C. for 30 minutes, quenched with water, and extracted with ethyl acetate (2×50 mL). The combined organic phases were washed with water and brine, dried over sodium sulfate, and concentrated. The residue was purified by silica gel column chromatography (petroleum ether/ethyl acetate=98:1) to give the title compound (2.8 g, 70%) as a yellow oil. MS (ESI): 400, 402 m/z [M+H]⁺, retention time: 2.62 minutes, purity: 85% (214 nm) (LC-MS method 14).

The following intermediate was prepared based on the procedures described for Intermediate 33A.

| Inter. | | | |
|---|---|---|---|
| No. | Structure | Name | MS m/z [M + H]⁺ |
| 33A-1 | 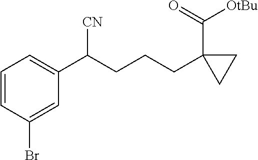 [SMILES: CC(C)(C)OC(=O)C1(CCCC(C#N)c2cccc(Br)c2)CC1] | tert-butyl 1-(4-(3-bromophenyl)-4- cyanobutyl)cyclopropane-1- carboxylate | 400, 402 [M + Na]⁺ |
| 33A-2 | 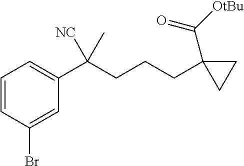 [SMILES: CC(C)(C)OC(=O)C1(CCCC(C)(C#N)c2cccc(Br)c2)CC1] | tert-butyl 1-(4-(3-bromophenyl)-4- cyanopentyl)cyclopropane-1- carboxylate | 414, 416 [M + Na]⁺ |

### Intermediate 33B: 2-(3-Bromo-2-fluorophenyl)-6-hydroxyhexanoic acid

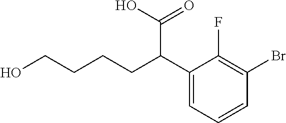 [SMILES: O=C(O)C(CCCCO)c1cccc(Br)c1F]

A mixture of 2-(3-bromo-2-fluorophenyl)-6-((tert-butyldimethylsilyl)oxy)hexanenitrile (Intermediate 33A, 2.8 g, 0.00699 mol) in potassium hydroxide (2 M, 70 mL) and ethanol (30 mL) was heated at 80° C. for 16 hours and concentrated. The residue was neutralized with 1 M hydrochloric acid to pH 4-5 and extracted with ethyl acetate (2×50 mL). The combined organic phases were washed with water and brine, dried over sodium sulfate, and concentrated. The residue was purified by silica gel column chromatography (petroleum ether/ethyl acetate=1:1) to give the title compound (1.6 g, 75%) as a yellow solid. MS (ESI): 327, 329 m/z [M+Na]⁺, retention time: 1.73 minutes, purity: 93% (214 nm) (LC-MS method 14).

The following intermediates were prepared based on the procedures described for Intermediate 33B.

| Inter. | | | |
|---|---|---|---|
| No. | Structure | Name | MS m/z [M + H]⁺ |
| 33B-1 | 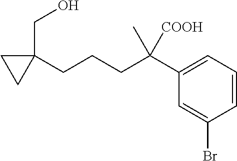 [SMILES: CC(CCCC1(CO)CC1)(C(=O)O)c1cccc(Br)c1] | 2-(3-bromophenyl)-5-(1- (hydroxymethyl)cyclopropyl)-2- methylpentanoic acid | 363, 365 [M + Na]⁺ |
| 33B-2 | 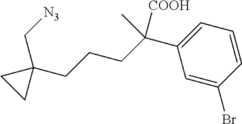 [SMILES: CC(CCCC1(CN=[N+]=[N-])CC1)(C(=O)O)c1cccc(Br)c1] | 5-(1-(azidomethyl)cyclopropyl)-2-(3- bromophenyl)-2-methylpentanoic acid | 388, 390 [M + Na]⁺ |

### Intermediate 34: 2-Fluoro-5-((6-fluoro-4-(4-hydroxybutyl)-1H-indol-5-yl)oxy)benzonitrile

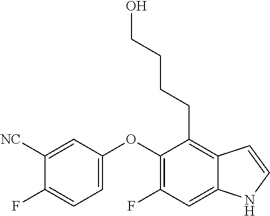 [SMILES: N#Cc1cc(Oc2c(F)cc3[nH]ccc3c2CCCCO)ccc1F]

To a stirred solution of 2-fluoro-5-((6-fluoro-4-(4-hydroxybutyl)-1-tosyl-1H-indol-5-yl)oxy)benzonitrile (Intermediate 29, 2.1 g, 4.23 mmol) in 30 mL of methanol was added potassium carbonate (1.8 g, 12.7 mmol). The mixture was stirred at 80° C. for 30 minutes and concentrated. The residue was partitioned between water (50 mL) and dichloromethane (50 mL). The separated organic layer, combined with two additional dichloromethane extracts (2×30 mL), was washed with brine, dried over magnesium sulfate, and concentrated. The crude product was purified by silica gel column chromatography (eluting with 0-40% ethyl acetate in petroleum ether) to give the title compound (800 mg, 58%) as a white foam. MS (ESI): 343 m/z [M+H]⁺, retention time: 1.92 minutes, purity: 96% (214 nm) (LC-MS method 5).

The following intermediates were prepared utilizing the procedures described for Intermediate 34.

| Inter. | | | |
|---|---|---|---|
| No. | Structure | Name | MS m/z [M + H]⁺ |
| 34-1 | 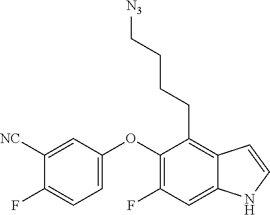 [SMILES: N#Cc1cc(Oc2c(F)cc3[nH]ccc3c2CCCCN=[N+]=[N-])ccc1F] | 5-((4-(4-azidobutyl)-6-fluoro-1H-indol- 5-yl)oxy)-2-fluorobenzonitrile | 390 [M + Na]⁺ |
| 34-2 | 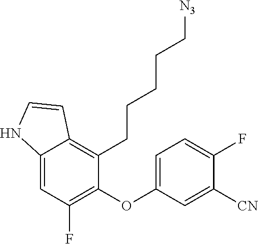 [SMILES: N#Cc1cc(Oc2c(F)cc3[nH]ccc3c2CCCCCN=[N+]=[N-])ccc1F] | 5-((4-(5-azidopentyl)-6-fluoro-1H- indol-5-yl)oxy)-2-fluorobenzonitrile | 404 [M + Na]⁺ |

### Intermediate 35: 5-Acetoxy-2-(3-(3-ethoxy-3-oxopropyl)phenyl)-2-methylpentanoic acid

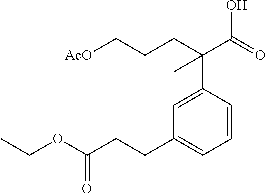 [SMILES: CCOC(=O)CCc1cccc(C(C)(CCCOC(C)=O)C(=O)O)c1]

To a stirred solution of ((E)-5-acetoxy-2-(3-(3-ethoxy-3-oxoprop-1-en-1-yl)phenyl)-2-methylpentanoic acid (Intermediate 35A, 1.3 g, 0.5 mmol) in methanol (15 mL) was added 10% palladium on carbon (50% wet, 130 mg). The mixture was stirred at room temperature under hydrogen balloon overnight, then filtered through a pad of celite. The filtrate was concentrated. The residue was purified by silica gel column chromatography (eluting with ethyl acetate/petroleum ether=1/2) to afford the title compound (1 g, 77%) as a yellow solid. MS (ESI): 351 m/z [M+H]⁺, retention time: 1.82 minutes, purity: 91% (214 nm) (LC-MS method 3).

The following intermediate was prepared based on the procedures described for Intermediate 35.

| Inter. | | | |
|---|---|---|---|
| No. | Structure | Name | MS m/z [M + H]⁺ |
| 35-1 | 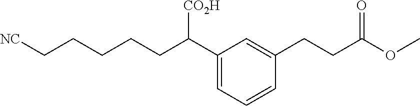 [SMILES: COC(=O)CCc1cccc(C(CCCCCC#N)C(=O)O)c1] | 7-cyano-2-(3-(3-methoxy-3- oxopropyl)phenyl)heptanoic acid | 318 |

### Intermediate 35A: (E)-5-Acetoxy-2-(3-(3-ethoxy-3-oxoprop-1-en-1-yl)phenyl)-2-methylpentanoic acid

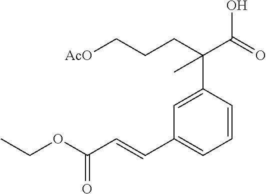 [SMILES: CCOC(=O)/C=C/c1cccc(C(C)(CCCOC(C)=O)C(=O)O)c1]

To a stirred and degassed solution of 5-acetoxy-2-(3-iodophenyl)-2-methylpentanoic acid (Intermediate 32-1, 3.4 g, 9 mmol) in N, N-dimethylformamide (40 mL) was added ethyl acrylate (1.8 g, 18 mmol), palladium (II) acetate (305 mg, 1.3 mmol), tri(o-tolyl)phosphine (305 mg, 0.9 mmol) and triethylamine (2.7 g, 27 mmol). The mixture was stirred in a sealed tube at 110° C. for 15 hours, cooled to room temperature, and diluted with ethyl acetate (100 mL). The solution was washed with water, brine, dried over sodium sulfate, and concentrated. The residue was purified by automated silica gel column chromatography (eluting with ethyl acetate/petroleum ether=1/3-1/2) to give the title compound (2.6 g, 74%) as a light-yellow solid. MS (ESI): 349 m/z [M+H]⁺, retention time: 1.83 minutes, purity: 93% (214 nm) (LC-MS method 3).

The following intermediate was prepared based on the procedures described for Intermediate 35A.

| Inter. | | | |
|---|---|---|---|
| No. | Structure | Name | MS m/z [M + H]⁺ |
| 35A-1 | 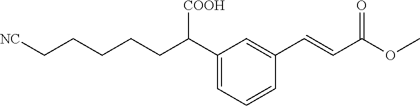 [SMILES: COC(=O)/C=C/c1cccc(C(CCCCCC#N)C(=O)O)c1] | (E)-7-cyano-2-(3-(3-methoxy-3-oxoprop- 1-en-1-yl)phenyl)heptanoic acid | 316 |

### Intermediate 36: (E)-N-methyl-3-(tributylstannyl)prop-2-en-1-amine

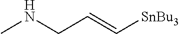

To a stirred solution of N-methylprop-2-yn-1-amine (1.2 g, 17.4 mmol) and tributylstannane (12.6 g, 43.4 mmol) in toluene (80 mL) was added 2,2′-azobis(2-methylpropionitrile) (713 mg, 4.34 mmol). The mixture was stirred at 105° C. overnight and concentrated. The residue was purified by automated flash chromatography (300 g silica gel column, eluting with 0-10% methanol in dichloromethane) to give the title compound (1.2 g, 19%, colorless oil) and its regio-isomer, N-methyl-2-tributylstannyl-prop-2-en-1-amine (3.3 g, 52%, colorless oil).

The title compound: ¹H NMR (400 MHz, CDCl₃) δ 6.64-5.13 (m, 2H), 3.38-3.17 (m, 2H), 2.43-2.38 (m, 3H), 1.62-1.41 (m, 6H), 1.38-1.25 (m, 6H), 0.96-0.81 (m, 15H).

N-methyl-2-tributylstannyl-prop-2-en-1-amine: ¹H NMR (400 MHz, CDCl₃) δ 6.19-5.90 (m, 2H), 3.31-3.21 (m, 2H), 2.43 (s, 3H), 1.59-1.39 (m, 6H), 1.39-1.24 (m, 6H), 0.92-0.82 (m, 15H).

### Intermediate 37: 2-Fluoro-5-((6-fluoro-4-(2-hydroxyethyl)-1H-indol-5-yl)oxy)benzonitrile

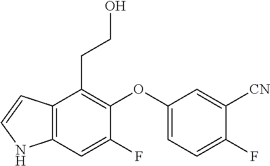 [SMILES: N#Cc1cc(Oc2c(F)cc3[nH]ccc3c2CCO)ccc1F]

To a stirred and cooled (0° C.) solution of 2-fluoro-5-[(6-fluoro-4-vinyl-1H-indol-5-yl)oxy]benzonitrile (Intermediate 9-1, 5 g, 17 mmol) in tetrahydrofuran (100 mL) was added borane-tetrahydrofuran complex (2 M in tetrahydrofuran, 25.3 mL, 50.6 mmol) dropwise. The mixture was stirred at room temperature for 3 hours, re-cooled to 0° C., and treated with sodium hydroxide (1 N in water, 50.6 mL, 50.6 mmol), followed by hydrogen peroxide (30% in water, 4.6 mL). The reaction mixture was stirred for 30 minutes, quenched with water (100 mL), and extracted with ethyl acetate (3×50 mL). The combined organic extracts were washed with brine (50 mL), dried over sodium sulfate, and concentrated. The residue was purified by silica gel column chromatography (eluting with ethyl acetate/petroleum ether (50:50)) to afford the title compound (1.5 g, 28%) as an oil. MS (ESI): 315 m/z [M+H]⁺, retention time: 1.88 minutes, purity: 90% (214 nm) (LC-MS method 9).

The following intermediate was prepared based on the procedures described for Intermediate 37.

| Inter. | | | |
|---|---|---|---|
| No. | Structure | Name | MS m/z [M + H]⁺ |
| 37-1 | 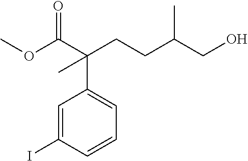 [SMILES: COC(=O)C(C)(CCC(C)CO)c1cccc(I)c1] | methyl 6-hydroxy-2-(3-iodophenyl)-2,5- dimethylhexanoate | 377 |

### Intermediate 38: 7-(((Benzyloxy)carbonyl)(methyl)amino)-2-(3-bromo-2-fluorophenyl)-5,5-dimethylheptanoic acid

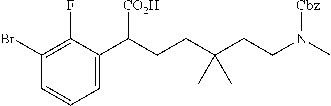 [SMILES: CN(CCC(C)(C)CCC(C(=O)O)c1cccc(Br)c1F)C(=O)OCc1ccccc1]

To a stirred solution of 2-(3-bromo-2-fluorophenyl)-5,5-dimethyl-7-(methylamino)heptanoic acid (Intermediate 38A, 1.7 g, 4.72 mmol) and potassium carbonate (1.3 g, 9.44 mmol) in tetrahydrofuran (30 mL) and water (10 mL) was added benzyl chloroformate (1.2 g, 7.08 mmol). The mixture was stirred at room temperature for 18 hours and concentrated. The residue was diluted with water (30 mL), acidified with 2 N hydrochloric acid to pH˜2, and extracted with ethyl acetate (3×50 mL). The combined organic layers were dried over sodium sulfate and concentrated. The residue was purified by automated flash chromatography (eluting with ethyl acetate/petroleum ether=1/1) to afford the title compound (435 mg, 19% two steps) as a colorless oil. MS (ESI): 494 m/z [M+H]⁺, retention time: 2.34 minutes, purity: 85% (254 nm) (LC-MS method 14).

The following intermediate was prepared based on the procedures described for Intermediate 38.

| Inter. | | | |
|---|---|---|---|
| No. | Structure | Name | MS m/z [M + H]⁺ |
| 38-1 | 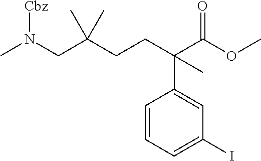 | methyl 6- (((benzyloxy)carbonyl)(methyl)amino)- 2-(3-iodophenyl)-2,5,5- trimethylhexanoate | 538 |

### Intermediate 38A: 2-(3-Bromo-2-fluorophenyl)-5,5-dimethyl-7-(methylamino)heptanoic acid

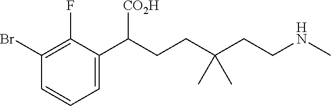 [SMILES: CNCCC(C)(C)CCC(C(=O)O)c1cccc(Br)c1F]

A solution of 2-(3-bromo-2-fluorophenyl)-5,5-dimethyl-7-(tosyloxy)heptanoic acid (Intermediate 1A-10, 2.37 g, 4.73 mmol) in methylamine (40 mL, 2.0 M in tetrahydrofuran) was stirred at 40° C. for 3 days in a sealed tube and concentrated. The crude title compound (1.7 g, crude) was obtained as a light-yellow oil, which was used for next step without further purification. MS (ESI): 360, 362 m/z [M+H]⁺, retention time: 2.58 minutes, purity: 80% (254 nm) (LC-MS method 14).

The following intermediate was prepared based on the procedures described for Intermediate 38A.

| Inter. | | | |
|---|---|---|---|
| No. | Structure | Name | MS m/z [M + H]⁺ |
| 38A-1 | 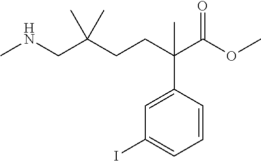 | methyl 2-(3-iodophenyl)-2,5,5- trimethyl-6-(methylamino)hexanoate | 404 |

### Intermediate 39: 2,2-Dimethylbutane-1,4-diyl bis(4-methylbenzenesulfonate)

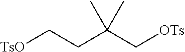 [SMILES: Cc1ccc(S(=O)(=O)OCCC(C)(C)COS(=O)(=O)c2ccc(C)cc2)cc1]

To a stirred and cooled (0° C.) solution of 2,2-dimethylbutane-1,4-diol (8.8 g, 74.6 mmol) in 50 mL of pyridine was added tosyl chloride (57 g, 298 mmol). The mixture was stirred at room temperature for 16 hours, quenched with water (100 mL) at 0° C., and extracted with ethyl acetate (3×100 mL). The combined organic phases were washed with 100 mL of 1 N sulfuric acid, brine, dried over sodium sulfate, and concentrated. The residue was purified by automated flash chromatography (eluting with 0-30% of ethyl acetate in petroleum ether) to give the title compound (6.4 g, 22%) as a white solid. MS (ESI): 449 m/z [M+Na]⁺, retention time: 2.23 minutes, purity: 94% (214 nm) (LC-MS method 9).

The following intermediates were prepared based on the procedures described for Intermediate 39.

| Inter. | | | |
|---|---|---|---|
| No. | Structure | Name | MS m/z [M + H]⁺ |
| 39-1 | 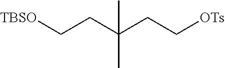 [SMILES: Cc1ccc(S(=O)(=O)OCCC(C)(C)CCO[Si](C)(C)C(C)(C)C)cc1] | 5-((tert-butyldimethylsilyl)oxy)-3,3- dimethylpentyl 4- methylbenzenesulfonate | 423 [M + Na]⁺ |
| 39-2 | 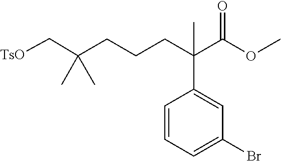 [SMILES: COC(=O)C(C)(CCCC(C)(C)COS(=O)(=O)c1ccc(C)cc1)c1cccc(Br)c1] | methyl 2-(3-bromophenyl)-2,6,6- trimethyl-7-(tosyloxy)heptanoate | 511, 513 |

### Intermediate 40: 2-(3-Bromophenyl)-5-(1-(hydroxymethyl)cyclopropyl)-2-methylpentanenitrile

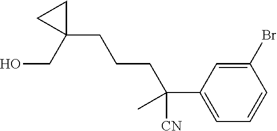 [SMILES: CC(C#N)(CCCC1(CO)CC1)c1cccc(Br)c1]

To a stirred and cooled (0° C.) solution of 1-(4-(3-bromophenyl)-4-cyanopentyl)cyclopropane-1-carboxylic acid (Intermediate 40A, 3.1 g, 0.00922 mol) in tetrahydrofuran (10 mL) was added borane-tetrahydrofuran solution (27.7 mL, 1M in tetrahydrofuran). The mixture stirred for 1 hour at room temperature, then quenched with methanol (50 mL), and concentrated. The residue was purified by silica gel column chromatography (petroleum ether/ethyl acetate=3:1) to give the title compound (1.8 g, 58%) as a yellow oil. MS (ESI): 344, 346 m/z [M+Na]⁺, retention time: 2.02 minutes, purity: 93% (214 nm) (LC-MS method 7).

### Intermediate 40A: 1-(4-(3-Bromophenyl)-4-cyanopentyl)cyclopropane-1-carboxylic acid

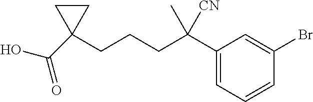 [SMILES: CC(C#N)(CCCC1(C(=O)O)CC1)c1cccc(Br)c1]

To a stirred solution of tert-butyl 1-(4-(3-bromophenyl)-4-cyanopentyl)cyclopropane-1-carboxylate (Intermediate 33A-2, 5.6 g, 0.0143 mol) in dichloromethane (20 mL) was added trifluoroacetic acid (5 mL). The mixture was stirred for 16 hours and concentrated. The residue was purified by silica gel column chromatography (petroleum ether/ethyl acetate=2:1) to give the title compound (3.1 g, 65%) as a yellow oil. MS (ESI): 336, 338m/z [M+H]⁺, retention time: 2.06 minutes, purity: 93% (214 nm) (LC-MS method 7).

The following intermediate was prepared based on the procedures described for Intermediate 40A.

| Inter. | | | |
|---|---|---|---|
| No. | Structure | Name | MS m/z [M + H]⁺ |
| 40A-1 | 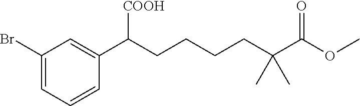 [SMILES: COC(=O)C(C)(C)CCCCC(C(=O)O)c1cccc(Br)c1] | 2-(3-bromophenyl)- 8-methoxy-7,7- dimethyl-8- oxooctanoic acid | 393, 395 [M + Na]⁺ |
| 40A-2 | 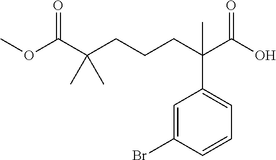 [SMILES: COC(=O)C(C)(C)CCCC(C)(C(=O)O)c1cccc(Br)c1] | 2-(3-bromophenyl)- 7-methoxy-2,6,6- trimethyl-7- oxoheptanoic acid | ¹H NMR (400 MHz, CDCl₃) δ 10.29 (s, 1H), 7.41 (t, J = 2.0 Hz, 1H), 7.35- 7.32 (m, 1H), 7.22-7.18 (m, 1H), 7.14 (t, J = 7.8 Hz, 1H), 3.56 (s, 3H), 1.96-1.87 (m, 1H), 1.83-1.76 (m, 1H), 1.49 (s, 3H), 1.47-1.41 (m, 2H), 1.09-1.00 (m, 8H). |
| 40A-3 | 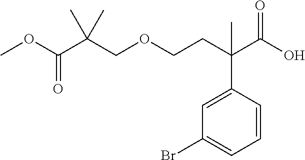 [SMILES: COC(=O)C(C)(C)COCCC(C)(C(=O)O)c1cccc(Br)c1] | 2-(3-bromophenyl)- 4-(3-methoxy-2,2- dimethyl-3- oxopropoxy)-2- methylbutanoic acid | 409, 411 [M + Na]⁺ |
| 40A-4 | 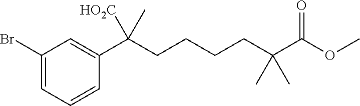 [SMILES: COC(=O)C(C)(C)CCCCC(C)(C(=O)O)c1cccc(Br)c1] | 2-(3-bromophenyl)- 8-methoxy-2,7,7- trimethyl-8- oxooctanoic acid | 407, 409 [M + Na]⁺ |
| 40A-5 | 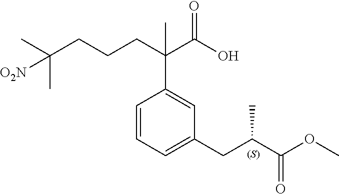 | 2-(3-((S)-3-methoxy- 2-methyl-3- oxopropyl)phenyl)- 2,6-dimethyl-6- nitroheptanoic acid | 402 [M + Na]⁺ |

### Intermediate 41: 4-Bromo-6-fluoro-5-(4-fluoro-3-(1H-pyrazol-3-yl)phenoxy)-1-tosyl-1H-indole

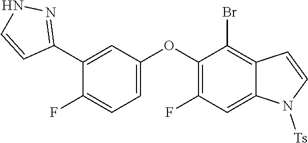 [SMILES: Cc1ccc(S(=O)(=O)n2ccc3c(Br)c(Oc4ccc(F)c(-c5cc[nH]n5)c4)c(F)cc32)cc1]

To a stirred solution of (E)-1-(5-((4-bromo-6-fluoro-1-tosyl-1H-indol-5-yl)oxy)-2-fluorophenyl)-3-(dimethylamino)prop-2-en-1-one (Intermediate 41C, 380 mg, 0.66 mmol) in ethanol (8 mL) was added hydrazine hydrate (0.13 mL, 98% aqueous solution, 3.28 mmol). The reaction was stirred at 50° C. for 3 hours and concentrated. The residue was partitioned between water (20 mL) and ethyl acetate (50 mL). The separated organic layer was washed with brine (20 mL), dried over sodium sulfate, and concentrated. The residue was purified by silica gel column chromatography (20 g silica gel column, eluting with 0-60% ethyl acetate in petroleum ether) to give the title compound (331 mg, 92%) as a white solid. MS (ESI): 544, 546 m/z [M+H]⁺, retention time: 2.24 minutes, purity: >99% (214 nm) (LC-MS method 7).

The following intermediate was prepared based on the procedures described for Intermediate 41.

| Inter. | | | |
|---|---|---|---|
| No. | Structure | Name | MS m/z [M + H]⁺ |
| 41-1 | 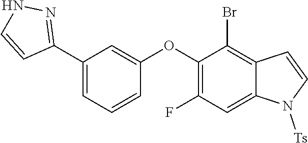 [SMILES: Cc1ccc(S(=O)(=O)n2ccc3c(Br)c(Oc4cccc(-c5cc[nH]n5)c4)c(F)cc32)cc1] | 5-(3-(1H-pyrazol-3-yl)phenoxy)-4- bromo-6-fluoro-1-tosyl-1H-indole | 526, 528 |

### Intermediate 41A: 1-(5-((4-Bromo-6-fluoro-1H-indol-5-yl)oxy)-2-fluorophenyl)ethan-1-one

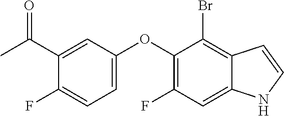 [SMILES: CC(=O)c1cc(Oc2c(F)cc3[nH]ccc3c2Br)ccc1F]

To a stirred solution of 5-((4-bromo-6-fluoro-1H-indol-5-yl)oxy)-2-fluorobenzonitrile (Intermediate 2, 2.0 g, 5.73 mmol) in toluene (30 mL) was added methylmagnesium bromide (3 M in diethyl ether, 7.64 mL, 22.9 mmol). The mixture was stirred at 110° C. for 18 hours, cooled to 0° C. and acidified with 10% hydrochloric acid. The solution refluxed for 1 hour, cooled to room temperature and extracted with ethyl acetate (3×50 mL). The combined organic extracts were washed with brine, dried over sodium sulfate, and concentrated. The residue was purified by automated flash chromatography (40 g silica gel column, eluting with 20-40% ethyl acetate in petroleum ether) to give the title compound (600 mg, 26%) as a light-yellow oil. MS (ESI): 366, 368 m/z [M+H]⁺, retention time: 2.09 minutes, purity: 97% (214 nm) (LC-MS method 5).

### Intermediate 41B: 1-(5-((4-Bromo-6-fluoro-1-tosyl-1H-indol-5-yl)oxy)-2-fluorophenyl)ethan-1-one

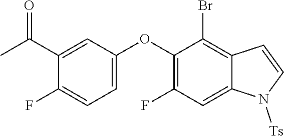 [SMILES: CC(=O)c1cc(Oc2c(F)cc3c(ccn3S(=O)(=O)c3ccc(C)cc3)c2Br)ccc1F]

To a stirred solution of 1-(5-((4-bromo-6-fluoro-1H-indol-5-yl)oxy)-2-fluorophenyl)ethan-1-one (Intermediate 41A, 2.75 g, purity 90%, 6.76 mmol) in tetrahydrofuran (50 mL) was added 4-methylbenzenesulfonyl chloride (1.93 g, 10.1 mmol), triethylamine (1.03 g, 10.1 mmol) and N,N-dimethylpyridin-4-amine (0.413 g, 3.38 mmol). The reaction was stirred at room temperature overnight, then diluted with water (100 mL), and extracted with ethyl acetate (3×70 mL). The combined organic layers were washed with brine, dried over sodium sulfate, and concentrated. The residue was suspended in petroleum ether: ethyl acetate=10: 1 (30 mL) and stirred for 30 minutes. The solid was collected by filtration to afford the title compound (400 mg, 11%) as a white solid. MS (ESI): 542, 544 m/z [M+Na]⁺, retention time: 2.38 minutes, purity: 85% (214 nm) (LC-MS method 7).

The following intermediate was prepared based on the procedures described for Intermediate 41B.

| Inter. | | | |
|---|---|---|---|
| No. | Structure | Name | MS m/z [M + H]⁺ |
| 41B-1 | 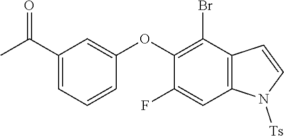 [SMILES: CC(=O)c1cccc(Oc2c(F)cc3c(ccn3S(=O)(=O)c3ccc(C)cc3)c2Br)c1] | 1-(3-((4-bromo-6-fluoro-1-tosyl- 1H-indol-5-yl)oxy)phenyl)ethan-1- one | 524, 526 [M + Na]⁺ |

### Intermediate 41C: (E)-1-(5-((4-Bromo-6-fluoro-1-tosyl-1H-indol-5-yl)oxy)-2-fluorophenyl)-3-(dimethylamino)prop-2-en-1-one

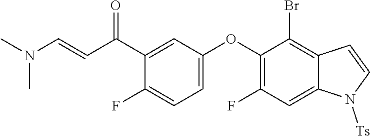 [SMILES: Cc1ccc(S(=O)(=O)n2ccc3c(Br)c(Oc4ccc(F)c(C(=O)/C=C/N(C)C)c4)c(F)cc32)cc1]

To a solution of 1-(5-((4-bromo-6-fluoro-1-tosyl-1H-indol-5-yl)oxy)-2-fluorophenyl)ethan-1-one (Intermediate 41B, 400 mg, 0.7 mmol) in N,N-dimethylformamide (5 mL) was added 1,1-dimethoxy-N,N-dimethyl-methanamine (0.12 mL, 0.9 mmol). The reaction was stirred at 90° C. for 2 hours and concentrated. The obtained crude title compound (500 mg, yellow solid) was used for next step without further purification. MS (ESI): 575, 577 m/z [M+H]⁺, retention time: 2.19 minutes, purity: 90% (254 nm) (LC-MS method 7).

### Intermediate 42: Ethyl (E)-3-(6-fluoro-5-(4-fluoro-3-(1H-pyrazol-3-yl)phenoxy)-1-tosyl-1H-indol-4-yl)acrylate

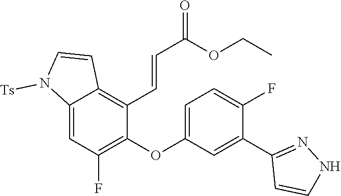 [SMILES: CCOC(=O)/C=C/c1c(Oc2ccc(F)c(-c3cc[nH]n3)c2)c(F)cc2c1ccn2S(=O)(=O)c1ccc(C)cc1]

To a stirred solution of 4-bromo-6-fluoro-5-(4-fluoro-3-(1H-pyrazol-3-yl)phenoxy)-1-tosyl-1H-indole (Intermediate 41, 1.45 g, 2.61 mmol) in N,N-dimethylformamide (15 mL) was added palladium (II) acetate (58.6 mg, 0.261 mmol), tris-o-tolyl-phosphine (159 mg, 0.522 mmol), ethyl acrylate (784 mg, 7.83 mmol) and triethylamine (1.32 g, 13.1 mmol). The reaction was stirred in a sealed tube under nitrogen atmosphere at 120° C. for 2 hours, cooled to room temperature, diluted with ethyl acetate (150 mL). The solution was washed with water, saturated lithium chloride, dried over sodium sulfate, and concentrated. The residue was purified by automated silica gel column chromatography (20 g silica gel column, eluting with 30% ethyl acetate in petroleum ether) to give the title compound (971 mg, 57%) as light-yellow solid. MS (ESI): 564 m/z [M+H]⁺, retention time: 2.29 minutes, purity: 86% (214 nm) (LC-MS method 7).

### Intermediate 43: Benzyl (2,2-dimethyl-6-oxohexyl)(methyl)carbamate

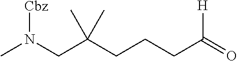 [SMILES: CN(CC(C)(C)CCCC=O)C(=O)OCc1ccccc1]

To a stirred solution of benzyl N-(6,7-dihydroxy-2,2-dimethyl-heptyl)-N-methylcarbamate (Intermediate 43B, 9 g, 27.8 mmol) in acetone (50 mL) and water (50 mL) was added sodium periodate (11.9 g, 55.7 mmol). The mixture was stirred at room temperature for 2 hours, then diluted with water (200 mL), and extracted with ethyl acetate (3×100 mL). The combined organic phase was washed with brine (100 mL), dried over sodium sulfate, and concentrated. The residue was purified by silica gel column chromatography (eluting with ethyl acetate/petroleum ether (20:80)) to afford the title compound (6 g, 74% for 2 steps) as an oil. MS (ESI): 292 m/z [M+H]⁺, retention time: 1.97 minutes, purity: 90% (214 nm) (LC-MS method 9).

The following intermediate was prepared based on the procedures described for Intermediate 43.

| Inter. | | | |
|---|---|---|---|
| No. | Structure | Name | MS m/z [M + H]⁺ |
| 43-1 | 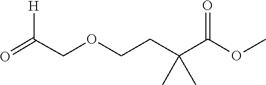 | methyl 2,2- dimethyl-4-(2- oxoethoxy)butanoate | ¹HNMR (400 MHZ, CDCl₃): δ 9.69 (s, 1H), 4.03 (s, 2H), 3.66 (s, 3H), 3.58 (t, J = 6.8 Hz, 2H), 1.93 (t, J = 6.8 Hz, 2H), 1.22 (s, 6H) ppm. |

### Intermediate 43A: Benzyl (2,2-dimethylhept-6-en-1-yl)(methyl)carbamate

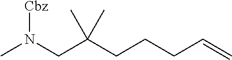 [SMILES: C=CCCCC(C)(C)CN(C)C(=O)OCc1ccccc1]

To a stirred solution of 2,2-dimethylhept-6-en-1-ol (Intermediate 30B-1, 12 g, 84 mmol) in dichloromethane (150 mL) was added pyridinium chlorochromate (36.4 g, 170 mmol) and silica gel (100-200 m, 36.4 g). The mixture was stirred at room temperature for 2 hours and filtered through a pad of Celite. The filtrate was concentrated to provide 2,2-dimethylhept-6-enal (crude, 12 g).

To a stirred solution of the above 2,2-dimethylhept-6-enal (12 g, crude) in dichloromethane (200 mL) and methanol (200 mL) was added methylamine in tetrahydrofuran (5 M, 86 mL, 428 mmol) and acetic acid (500 mg). The mixture was stirred at room temperature for 16 hours, cooled to 0° C., then treated with sodium borohydride (3.9 g, 103 mmol). After completion (monitored by TLC), the reaction was quenched with saturated sodium bicarbonate solution (100 mL). The mixture was concentrated to remove the organic solvent. The aqueous residue was extracted with ethyl acetate (3×100 mL). The combined organic layers were washed with brine, dried over sodium sulfate and concentrated to afford crude N,2,2-trimethylhept-6-en-1-amine (13.3 g, crude).

To a stirred solution of the above N,2,2-trimethylhept-6-en-1-amine (13.3 g, crude) in water (100 mL) and tetrahydrofuran (50 mL) was added benzyl chloroformate (14.6 g, 85.7 mmol). The mixture was stirred at room temperature for 2 hours, then diluted with water (200 mL) and extracted with ethyl acetate (3×100 mL). The combined organic phases were washed with water, brine, dried over sodium sulfate. and concentrated. The residue was purified by silica gel column chromatography (eluting with ethyl acetate/petroleum ether (20:80)) to afford the title compound as oil (8 g, 32% for 3 steps). MS (ESI): 290 m/z [M+H]⁺, retention time: 2.04 minutes, purity: 66% (214 nm) (LC-MS method 16).

### Intermediate 43B: Benzyl (6,7-dihydroxy-2,2-dimethylheptyl)(methyl)carbamate

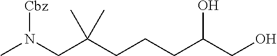 [SMILES: CN(CC(C)(C)CCCC(O)CO)C(=O)OCc1ccccc1]

To a stirred solution of benzyl N-(2,2-dimethylhept-6-enyl)-N-methylcarbamate (Intermediate 43A, 8 g, 27.6 mmol) in tetrahydrofuran (50 mL) and water (15 mL) was added osmium (VIII) tetroxide (50 drops) and 4-methylmorpholine N-oxide monohydrate (7.47 g, 55.3 mmol). The mixture was stirred at room temperature for 16 hours, diluted with water (200 mL), and extracted with ethyl acetate (3×100 mL). The combined organic phases were washed with brine (100 mL), dried over sodium sulfate, and concentrated to give the crude title compound (9 g, crude), which was used for the next step without further purification. MS (ESI): 324 m/z [M+H]⁺, retention time: 1.87 minutes, purity: 71% (214 nm) (LC-MS method 9).

The following intermediate was prepared based on the procedures described for Intermediate 43B.

| Inter. | | | |
|---|---|---|---|
| No. | Structure | Name | MS m/z [M + H]⁺ |
| 43B-1 | 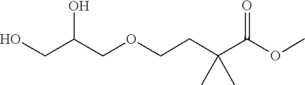 | methyl 4-(2,3- dihydroxypropoxy)- 2,2- dimethylbutanoate | ¹H NMR (400 MHz, CDCl₃) δ 3.81-3.78 (m, 1H), 3.67 (s, 3H), 3.65-3.39 (m, 4H), 3.05- 3.00 (m, 1H), 2.28-2.26 (m, 1H), 1.93-1.74 (m, 2H), 1.21 (s, 6H) ppm. |

### Intermediate 44: Benzyl (6-bromo-6-(3-bromophenyl)-2,2-dimethylhexyl)(methyl)carbamate

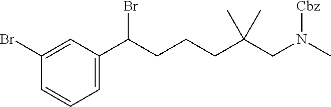 [SMILES: CN(CC(C)(C)CCCC(Br)c1cccc(Br)c1)C(=O)OCc1ccccc1]

To a stirred and cooled (0° C.) solution of benzyl (6-(3-bromophenyl)-6-hydroxy-2,2-dimethylhexyl)(methyl)carbamate (Intermediate 44A, 3 g, 6.7 mmol) in dichloromethane (100 mL) was added phosphorus tribromide (5.4 g, 20 mmol) at 0° C. The mixture was stirred at room temperature for 16 hours, then poured into ice water (100 mL), and extracted with ethyl acetate (3×100 mL). The combined organic phases were washed with water, brine, dried over sodium sulfate, and concentrated. The residue was purified by silica gel column chromatography (eluting with ethyl acetate/petroleum ether (20:80)) to afford the title compound (2.6 g, 76%) as oil. MS (ESI): 534 m/z [M+Na]⁺, retention time: 2.53 minutes, purity: 90% (214 nm) (LC-MS method 9).

The following intermediate was prepared based on the procedures described for Intermediate 44.

| Inter. | | | |
|---|---|---|---|
| No. | Structure | Name | MS m/z [M + H]⁺ |
| 44-1 | 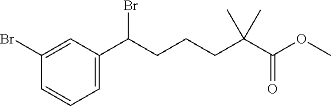 [SMILES: COC(=O)C(C)(C)CCCC(Br)c1cccc(Br)c1] | methyl 6- bromo-6-(3- bromophenyl)- 2,2- dimethylhexanoate | ¹H NMR (400 MHz, CDCl₃) 7.53-7.51 (m, 1H), 7.42-7.39 (m, 1H), 7.31-7.28 (m, 1H), 7.23-7.17 (m 1H), 4.87-4.83 (m, 1H), 3.63 (s, 3H), 2.25-2.16 (m, 1H), 2.09-2.00 (m, 1H), 1.59-1.53 (m, 2H), 1.28-1.24 (m, 2H), 1.15 (s, 3H), 1.14 (s, 3H) ppm. |

### Intermediate 44A: Benzyl (6-(3-bromophenyl)-6-hydroxy-2,2-dimethylhexyl)(methyl)carbamate

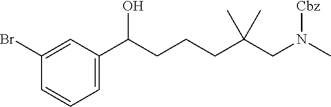 [SMILES: CN(CC(C)(C)CCCC(O)c1cccc(Br)c1)C(=O)OCc1ccccc1]

To a suspension of magnesium (1.98 g, 82 mmol) in tetrahydrofuran (10 mL) was added iodine (catalytic amount) followed by 1,3-dibromobenzene (14.6 g, 62 mmol) in tetrahydrofuran (50 mL) dropwise at room temperature. The solution was stirred gently at ambient temperature for 30 minutes, then added additional tetrahydrofuran (20 mL). The solution was refluxed for 3 hours and cooled to ambient temperature. The obtained Grignard reagent was then added to a solution of benzyl (2,2-dimethyl-6-oxohexyl)(methyl)carbamate (6 g, 20.6 mmol) in tetrahydrofuran (20 mL) at 0° C. The mixture was stirred for 2 hours, quenched with saturated ammonium chloride solution, and extracted with ethyl acetate (3×100 mL). The combined organic phase was washed with water, brine, dried over sodium sulfate, and concentrated. The crude was purified by column chromatography on silica gel using ethyl acetate/petroleum ether (40:60) to afford the title compound as oil (3 g, 32.5%). MS (ESI): 470, 472 m/z [M+Na]⁺, retention time: 2.33 minutes, purity: 85% (214 nm) (LC-MS method 9).

The following intermediate was prepared based on the procedures described for Intermediate 44A.

| Inter. | | | |
|---|---|---|---|
| No. | Structure | Name | MS m/z [M + H]⁺ |
| 44A-1 | 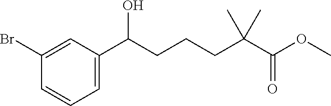 [SMILES: COC(=O)C(C)(C)CCCC(O)c1cccc(Br)c1] | methyl 6-(3- bromophenyl)- 6-hydroxy-2,2- dimethylhexaonate | 311, 313 [M − H2O + H]⁺ |

### Intermediate 46: 5-Iodo-2,2-dimethylpentanenitrile

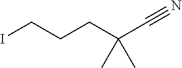

To a stirred solution of 5-chloro-2,2-dimethylpentanenitrile (36 g, 247 mmol) in acetone (300 mL) was added sodium iodide (111 g, 742 mmol). The reaction mixture was stirred for 16 hours at 60° C. and concentrated. The residue was diluted with ethyl acetate (300 mL) and washed with water, brine, dried over magnesium sulfate, and concentrated. The residue was purified by automated silica gel column chromatography (eluting with 0-20% petroleum ether-ethyl acetate) to give 5-iodo-2,2-dimethyl-pentanenitrile (47 g, 79%) as an oil. MS (ESI): 238 m/z [M+H]⁺, retention time: 1.98 minutes, purity: 98% (254 nm) (LC-MS method 4).

The following intermediate was prepared based on the procedures described for Intermediate 46.

| Inter. | | | |
|---|---|---|---|
| No. | Structure | Name | MS m/z [M + H]⁺ |
| 46-1 | 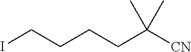 | 6-iodo-2,2- dimethylhexanitrile | ¹H NMR (400 MHz, CDCl₃) δ 3.21 (t, J = 6.9 Hz, 2H), 1.94-1.81 (m, 2H), 1.67-1.49 (m, 4H), 1.36 (s, 6H). |

### Intermediate 46A: 5-Chloro-2,2-dimethylpentanenitrile

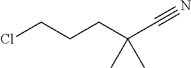

To a stirred solution of 1 M lithium bis(trimethylsilyl)amide (362 mL, 362 mmol) in tetrahydrofuran was added isobutyronitrile (25 g, 362 mmol) and 1-bromo-3-chloropropane (40 mL, 398 mmol) sequentially. The mixture was stirred at 70° C. for 16 hours, cooled to room temperature, quenched with water, and extracted with dichloromethane. The combined organic layers were dried over sodium sulfate and concentrated to give the crude title compound (36 g, crude) as an oil.

The following intermediate was prepared based on the procedures described for Intermediate 46A.

| Inter. | | | |
|---|---|---|---|
| No. | Structure | Name | MS m/z [M + H]⁺ |
| 46A-1 | 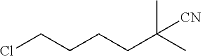 | 6-chloro-2,2- dimethylhexanenitrile | ¹H NMR (400 MHz, CDCl₃) δ 3.21 (t, J = 6.9 Hz, 2H), 1.94-1.79 (m, 2H), 1.68-1.49 (m, 4H), 1.36 (s, 6H). |

### Intermediate 47: 2-(2-Chloroethoxy)-2-methylpropanenitrile

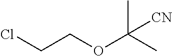

Zinc chloride (67.3 g, 0.494 mole) was fused by heating under vacuum. After cooling to room temperature, 2-hydroxy-2-methyl-propanenitrile (42 g, 0.494 mole) and 2-chloroethanol (60 g, 0.745 mole) was added. The reaction mixture was stirred for 10 hours at 60° C., cooled to room temperature, and quenched with water (300 mL). The solution was extracted with dichloromethane (5×100 mL). The combined organic extracts were washed with brine, dried over sodium sulfate, and concentrated. The residue was purified by vacuum distillation (10 mm Hg) to afford the title compound (the fractional boiling point between 65 to 75° C.) (36.4 g, 50%) as a colorless oil. ¹H NMR (400 MHz, CDCl₃) δ 3.85 (t, J=5.7 Hz, 2H), 3.65 (t, J=5.7 Hz, 2H), 1.61 (s, 6H).

### Intermediate 48: Methyl 4-((1-(((benzyloxy)carbonyl)(methyl)amino)-2-methylpropan-2-yl)oxy)-2-(3-iodophenyl)-2-methylbutanoate

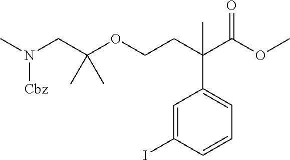

To a stirred and cooled (0° C.) solution of methyl 4-((1-(((benzyloxy)carbonyl)amino)-2-methylpropan-2-yl)oxy)-2-(3-iodophenyl)-2-methylbutanoate (Intermediate 48B, 1.44 g, 2.60 mmol) in tetrahydrofuran (15 mL) was added sodium hydride (60%, 214 mg). The mixture was stirred at 0° C. for 15 minutes, then treated with iodomethane (0.5 mL), and stirred at room temperature for 5 hours. The mixture was quenched with saturated ammonium chloride (50 mL), extracted with ethyl acetate (2×30 mL). The combined organic extracts were washed with brine (30 mL), dried over sodium sulfate, and concentrated. The residue was purified by automated flash chromatography (40 g silica gel column, eluting with 0-30% ethyl acetate in petroleum ether) to give the title compound (1.18 g, 80%) as a solid. MS (ESI): 576 m/z [M+Na]⁺, retention time: 2.44 minutes, purity: 85% (254 nm) (LC-MS method 9).

### Intermediate 48A: Methyl 4-((1-amino-2-methylpropan-2-yl)oxy)-2-(3-iodophenyl)-2-methylbutanoate

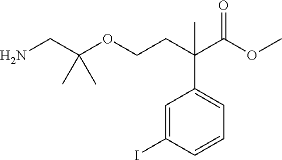 [SMILES: COC(=O)C(C)(CCOC(C)(C)CN)c1cccc(I)c1]

To a stirred and cooled (−78° C.) solution of 4-((2-cyanopropan-2-yl)oxy)-2-(3-iodophenyl)-2-methylbutanoic acid (Intermediate 19-13, 3 g, 7.75 mmol) in toluene (40 mL) was added a solution of diisobutylaluminium hydride in hexane (1 N, 38.7 mL, 38.7 mmol). The reaction was stirred at −78° C. for 2 hours, and at room temperature overnight, then quenched with methanol (100 mL) and concentrated sulfuric acid (10 mL). The mixture was refluxed for 2 days, then diluted with water (75 mL), and concentrated to remove organic solvents. The aqueous residue was basified with solid sodium bicarbonate to pH >9, extracted with ethyl acetate (3×75 mL). The combined organic extraction was washed with brine, dried over sodium sulfate, and concentrated. The residue was purified by automated flash chromatography (80 g silica gel column, eluting with 0-10% methanol in dichloromethane) to give the title compound (1.35 g, 43%) as an oil. ¹H NMR (400 MHz, CDCl₃) δ 7.65-7.63 (m, 1H), 7.60-7.56 (m, 1H), 7.30-7.25 (m, 1H), 7.06 (t, J=7.9 Hz, 1H), 4.16-4.06 (m, 2H), 3.66 (s, 3H), 3.42-3.36 (m, 2H), 2.82-2.80 (m, 2H), 2.50-2.37 (m, 1H), 2.14-2.01 (m, 1H), 1.54 (s, 3H), 1.19-1.16 (m, 6H).

### Intermediate 48B: Methyl 4-((1-(((benzyloxy)carbonyl)amino)-2-methylpropan-2-yl)oxy)-2-(3-iodophenyl)-2-methylbutanoate

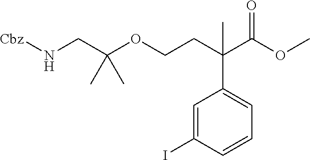 [SMILES: COC(=O)C(C)(CCOC(C)(C)CNC(=O)OCc1ccccc1)c1cccc(I)c1]

To a stirred solution of methyl 4-((1-amino-2-methylpropan-2-yl)oxy)-2-(3-iodophenyl)-2-methylbutanoate (Intermediate 48A, 1.35 g, 3.33 mmol) in dichloromethane (20 mL) was added triethylamine (700 μL, 5.0 mmol) and benzyl chloroformate (739 mg, 3.4 mmol). The mixture was stirred at room temperature overnight, then diluted with 100 mL of dichloromethane. The solution was washed with saturated sodium bicarbonate, brine, dried over sodium sulfate, and concentrated. The residue was purified by automated flash chromatography (40 g silica gel column, eluting with 0-30% ethyl acetate in petroleum) to afford the title compound (1.44 g, 80%) as an oil. ¹H NMR (400 MHz, CDCl₃) δ 7.66-7.64 (m, 1H), 7.56 (d, J=7.9 Hz, 1H), 7.42-7.27 (m, 6H), 7.04 (t, J=7.9 Hz, 1H), 5.24-5.20 (m, 1H), 5.12 (s, 2H), 3.62 (s, 3H), 3.40-3.29 (m, 2H), 3.19-3.10 (m, 2H), 2.48-2.30 (m, 1H), 2.08-2.00 (m, 1H), 1.53 (s, 3H), 1.12-1.08 (m, 6H).

### Intermediate 49: Benzyl (2-((3-(3-iodophenyl)-3-methyl-4-oxopentyl)oxy)-2-methylpropyl)(methyl)carbamate

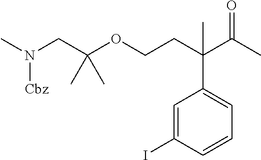 [SMILES: CC(=O)C(C)(CCOC(C)(C)CN(C)C(=O)OCc1ccccc1)c1cccc(I)c1]

To a stirred solution of benzyl (2-((4-hydroxy-3-(3-iodophenyl)-3-methylpentyl)oxy)-2-methylpropyl)(methyl)carbamate (Intermediate 49C, 0.8 g, 1.48 mmol) in dimethyl sulfoxide (13 mL) was added stabilized 2-iodoxybenzoic acid (61717-82-6, 46%) (1.25 g, 2.05 mmol). The mixture was stirred at 40° C. for 2 hours, then diluted with ethyl acetate (80 mL). The solution was washed with 1N sodium hydroxide, brine (2×20 mL), dried over sodium sulfate, and concentrated. The residue was purified by automated flash chromatography (40 g silica gel column, eluting with 0-40% ethyl acetate in petroleum ether) to give the title compound (0.63 g, 79% three steps) as an oil. MS (ESI): 560 m/z [M+Na]⁺, retention time: 2.44 minutes, purity: 85% (214 nm) (LC-MS method 9).

The following intermediates were prepared based on the procedures described for Intermediate 49.

| Inter. | | | |
|---|---|---|---|
| No. | Structure | Name | MS m/z [M + H]⁺ |
| 49-1 | 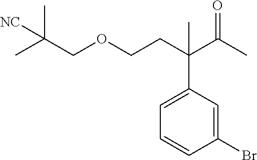 [SMILES: CC(=O)C(C)(CCOCC(C)(C)C#N)c1cccc(Br)c1] | 3-((3-(3-bromophenyl)-3- methyl-4-oxopentyl)oxy)-2,2- dimethylpropanenitrile | 352, 354 |
| 49-2 | 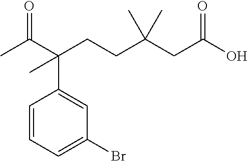 [SMILES: CC(=O)C(C)(CCC(C)(C)CC(=O)O)c1cccc(Br)c1] | 6-(3-bromophenyl)-3,3,6- trimethyl-7-oxooctanoic acid | 337, 339 [M − H2O + H]⁺ |

### Intermediate 49A: Benzyl (2-(4-hydroxy-3-(3-iodophenyl)-3-methylbutoxy)-2-methylpropyl)(methyl)carbamate

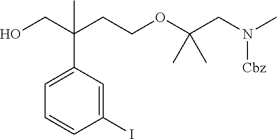 [SMILES: CN(CC(C)(C)OCCC(C)(CO)c1cccc(I)c1)C(=O)OCc1ccccc1]

To a stirred and cooled (0° C.) solution of 4-((1-(((benzyloxy)carbonyl)(methyl)amino)-2-methylpropan-2-yl)oxy)-2-(3-iodophenyl)-2-methylbutanoic acid (Intermediate 19-14, 1.05 g, 1.95 mmol) in tetrahydrofuran (20 mL) was added borane-methyl sulfide complex (2 M in tetrahydrofuran, 4.87 mL, 9.74 mmol). The reaction was stirred at room temperature for 4 hours, then quenched with water (100 mL), and extracted with ethyl acetate (3×30 mL). The combined organic extracts were washed with brine, dried over sodium sulfate, and concentrated. The residue was purified by automated flash chromatography (40 g silica gel column, eluting with 0-60% ethyl acetate in petroleum ether) to give the title compound (0.83 g, 81%) as an oil. MS (ESI): 526 m/z [M+H]⁺, retention time: 2.31 minutes, purity: 94% (254 nm) (LC-MS method 9).

### Intermediate 49B: Benzyl (2-(3-(3-iodophenyl)-3-methyl-4-oxobutoxy)-2-methylpropyl)(methyl)carbamate

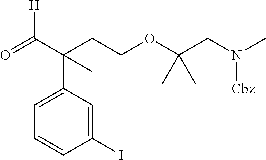 [SMILES: CN(CC(C)(C)OCCC(C)(C=O)c1cccc(I)c1)C(=O)OCc1ccccc1]

To a stirred solution of benzyl (2-(4-hydroxy-3-(3-iodophenyl)-3-methylbutoxy)-2-methylpropyl)(methyl)carbamate (Intermediate 49A, 0.83 g, 1.58 mmol) in dimethyl sulfoxide (13 mL) was added stabilized 2-iodoxybenzoic acid (61717-82-6) (46%, 1.33 g, 2.2 mmol). The mixture was stirred at 40° C. for 2 hours then diluted with ethyl acetate (80 mL). The solution was washed with 1 N sodium hydroxide, brine, dried over sodium sulfate, and concentrated to give the crude title compound (0.8 g, crude) as an oil. MS (ESI): 524 m/z [M+H]⁺, retention time: 2.42 minutes, purity: 70% (214 nm) (LC-MS method 9),

### Intermediate 49C: Benzyl (2-((4-hydroxy-3-(3-iodophenyl)-3-methylpentyl)oxy)-2-methylpropyl)(methyl)carbamate

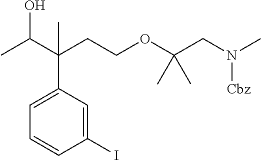 [SMILES: CC(O)C(C)(CCOC(C)(C)CN(C)C(=O)OCc1ccccc1)c1cccc(I)c1]

To a stirred and cooled (0° C.) solution of benzyl N-[2-[3-(3-iodophenyl)-3-methyl-4-oxo-butoxy]-2-methyl-propyl]-N-methyl-carbamate (Intermediate 49B, 0.8 g, 1.53 mmol) in tetrahydrofuran (7 mL) was added methyl magnesium bromide (3M in 2-methyl-tetrahydrofuran, 0.77 mL, 2.31 mmol). The mixture was stirred at 0° C. for 1 hour, quenched with saturated ammonium chloride (30 mL), and extracted with ethyl acetate (3×20 mL). The combined organic extracts were washed with brine (2×20 mL), dried over sodium sulfate, and concentrated to afford the crude title compound (0.8 g) as an oil. MS (ESI): 540 m/z [M+H]⁺, retention time: 2.37 minutes, purity: 67% (214 nm) (LC-MS method 9),

The following intermediates were prepared based on the procedures described for Intermediate 49C.

| Inter. | | | MS m/z |
|---|---|---|---|
| No. | Structure | Name | [M + H]⁺ |
| 49C-1 | 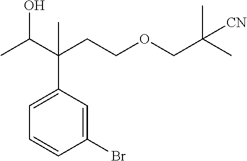 [SMILES: CC(O)C(C)(CCOCC(C)(C)C#N)c1cccc(Br)c1] | 3-((3-(3-bromophenyl)-4- hydroxy-3- methylpentyl)oxy)-2,2- dimethylpropanenitrile | 376, 378 [M + Na]⁺ |
| 49C-2 | 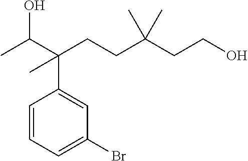 [SMILES: CC(O)C(C)(CCC(C)(C)CCO)c1cccc(Br)c1] | 6-(3-bromophenyl)-3,3,6- trimethyloctane-1,7-diol | 325, 327 [M − H2O + H]⁺ |

### Intermediate 50: 7-hydroxy-2-(3-iodophenyl)-2,6,6-trimethylheptanehydrazide

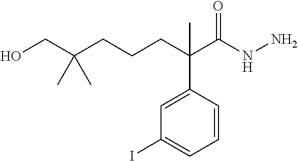 [SMILES: CC(C)(CO)CCCC(C)(C(=O)NN)c1cccc(I)c1]

To a stirred solution of 7-hydroxy-2-(3-iodophenyl)-2,6,6-trimethylheptanoic acid (Intermediate 32A-3, 4.6 g, 10.5 mmol) in N,N-dimethylformamide (50 ml) was added 1-hydroxybenzotriazole (1.7 g, 12.6 mmol), 1-ethyl-3-(3-dimethylaminopropyl)carbodiimide (2.4 g, 12.6 mmol). The mixture was stirred at room temperature for 2 hours, then cooled to 0° C., and treated with hydrazine hydrate (1.0 mL, 21.0 mmol). The mixture was stirred at room temperature for 1 hour and diluted with ethyl acetate (100 mL). The solution was washed with water, brine, dried over sodium sulfate, and concentrated. The residue was purified by automated flash chromatography (40 g silica gel column, eluting with 0-40% ethyl acetate in petroleum ether) to give the title compound (3.3 g, 62%) as oil. MS (ESI): 405 m/z [M+H]⁺, retention time: 1.79 minutes, purity: 79% (254 nm) (LC-MS method 4).

### Intermediate 51: 5-((4-Bromo-6-fluoro-1-(tetrahydro-2H-pyran-2-yl)-1H-benzo[d]imidazol-5-yl)oxy)-2-fluorobenzonitrile

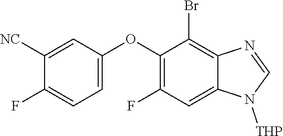

To a stirred solution of 5-((4-bromo-6-fluoro-1H-benzo[d]imidazol-5-yl)oxy)-2-fluorobenzonitrile (Intermediate 51C, 10 g, 28.5 mmol) in tetrahydrofuran (150 mL) was added 3,4-dihydro-2H-pyran (7.2 g, 85.5 mmol) and toluene sulfonic acid (245 mg, 1.43 mmol). The mixture was refluxed over the weekend and concentrated. The residue was purified by flash chromatography (120 g silica gel column, eluting with 0-70% ethyl acetate in petroleum ether) to give the title compound (6.95 g, 56% 4 steps) as a yellow solid. MS (ESI): 434, 436 m/z [M+H]⁺, retention time: 1.95 minutes, purity: 98% (214 nm) (LC-MS method 3).

### Intermediate 51A: 2-bromo-3,4-difluoro-6-nitroaniline

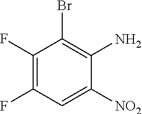 [SMILES: Nc1c([N+](=O)[O-])cc(F)c(F)c1Br]

To a stirred solution of 4,5-difluoro-2-nitroaniline (4.5 g, 25.9 mmol) in N,N-dimethylformamide (150 mL) was added N-bromosuccinimide (5.07 g, 28.5 mmol). The mixture was stirred at room temperature overnight, poured into 600 mL of water and extracted with ethyl acetate (3×100 mL). The combined organic extracts were washed with brine, dried over sodium sulfate, and concentrated to give the crude title compound (6.3 g, crude) as a solid. MS (ESI): 255, 253 m/z [M+H]⁺, retention time: 1.86 minutes, purity: 81% (254 nm) (LC-MS method 3).

### Intermediate 51B: 5-(3-amino-2-bromo-6-fluoro-4-nitrophenoxy)-2-fluorobenzonitrile

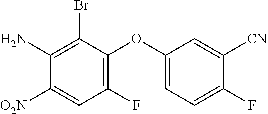 [SMILES: N#Cc1cc(Oc2c(F)cc([N+](=O)[O-])c(N)c2Br)ccc1F]

To a stirred solution of 2-bromo-3,4-difluoro-6-nitroaniline (Intermediate 51A, 6.3 g, 24.8 mmol) and 2-fluoro-5-hydroxybenzonitrile (3.57 g, 26 mmol) in N,N-dimethylformamide (150 mL) was added potassium carbonate (6.9 g, 49.6 mmol). The mixture was stirred at room temperature overnight, poured into 1000 mL of water and stirred for 30 minutes. The formed solid was collected by filtration and dried in vacuo to give the crude title compound (9 g, crude) as a solid. MS (ESI): 370, 372 m/z [M+H]⁺, retention time: 1.94 minutes, purity: 80% (214 nm) (LC-MS method 3).

### Intermediate 51C: 5-((4-bromo-6-fluoro-1H-benzo[d]imidazol-5-yl)oxy)-2-fluorobenzonitrile

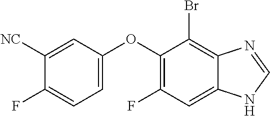 [SMILES: N#Cc1cc(Oc2c(F)cc3[nH]cnc3c2Br)ccc1F]

To a stirred solution of 5-(3-amino-2-bromo-6-fluoro-4-nitrophnoxy)-2-fluorobenzonitrile (9 g, 24.3 mmol) in isopropanol (150 mL) was added iron powder (13.6 g, 243 mmol) followed by ammonium chloride (13 g, 243 mmol) and formic acid (50 mL). The mixture was stirred at 85° C. for 3 hours and concentrated. The residue was poured into 200 mL of water and extracted with ethyl acetate (3×100 mL). The combined organic extracts were washed with brine, dried over sodium sulfate, and concentrated to give the crude title compound (9 g, crude) as a solid. MS (ESI): 350, 352 m/z [M+H]⁺, retention time: 1.73 minutes, purity: 87% (214 nm) (LC-MS method 3).

### Intermediate 52: benzyl (E)-3-(5-(3-cyano-4-fluorophenoxy)-6-fluoro-1H-benzo[d]imidazol-4-yl)acrylate

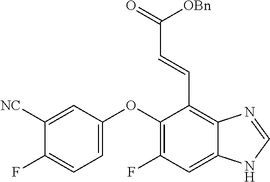 [SMILES: N#Cc1cc(Oc2c(F)cc3[nH]cnc3c2/C=C/C(=O)OCc2ccccc2)ccc1F]

A solution of benzyl (E)-3-(5-(3-cyano-4-fluorophenoxy)-6-fluoro-1-(tetrahydro-2H-pyran-2-yl)-1H-benzo[d]imidazol-4-yl)acrylate (Intermediate 4-2, 1 g, 1.94 mmol) in 4 N hydrogen chloride in dioxane (20 mL) was stirred at room temperature overnight and concentrated. The residue was diluted with ethyl acetate (100 mL), washed with saturated sodium bicarbonate, brine, dried over sodium sulfate, and concentrated. The crude product was purified by automated flash chromatography (40 g silica gel column, eluting with 0-100% ethyl acetate in petroleum ether) to give the title compound (810 mg, 96%) as a solid. MS (ESI): 432 m/z [M+H]⁺, retention time: 2.00 minutes, purity: 90% (214 nm) (LC-MS method 7).

### Intermediate 52: O-(2-Methoxyethyl)hydroxylamine

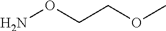

To a stirred suspension of 2-(2-methoxyethoxy)isoindoline-1,3-dione (Intermediate 52A, 8 g, 36.2 mmol) in methanol (50 mL) was added hydrazine hydrate (2.5 mL, 50.6 mmol). The mixture was heated to reflux for 4 hours. Then stirred at room temperature overnight and filtered. The filtrate was concentrated. The residue was diluted with ether (30 mL) and filtered again. The filtrate was concentrated. The process was repeat twice to give the title compound (2.3 g, 70%) as a colorless oil. ¹H NMR (400 MHz, CDCl₃) δ 3.82-3.86 (m, 2H), 3.56-3.59 (m, 2H), 3.39 (s, 3H) ppm.

### Intermediate 52A: 2-(2-Methoxyethoxy)isoindoline-1,3-dione

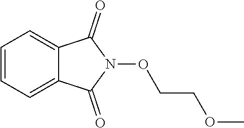 [SMILES: COCCON1C(=O)c2ccccc2C1=O]

To a stirred and cooled (0° C.) solution of 2-hydroxyisoindoline-1,3-dione (9.43 g, 57.8 mmol), 2-methoxyethanol (4 g, 52.6 mmol), and triphenylphosphine (17.9 g, 68.3 mmol) in 100 mL of tetrahydrofuran was added a solution of diethyl azodicarboxylate (13.8 g, 57.8 mmol) in 15 mL of tetrahydrofuran dropwise. The reaction was stirred at 0° C. for 10 minutes, then at room temperature overnight, and concentrated. The residue was purified by flash column chromatography (eluting with petroleum ether/ethyl acetate 7/3) to give the title compound (8 g, 74%) as a white solid. MS (ESI): 222 m/z [M+H]⁺, retention time: 1.73 minutes, purity: 80% (214 nm) (LC-MS method 5). ¹HNMR: (400 MHz, CDCl₃): δ 7.83-7.86 (m, 2H), 7.74-7.78 (m, 2H), 4.36 (t, J=4.4 Hz, 2H), 3.76 (t, J=4.4 Hz, 2H), 3.39 (s, 3H) ppm.

### Intermediate 53: 2-(4-(2-Ethoxy-2-oxoethyl)phenyl)-7-hydroxy-2,6,6-trimethylheptanoic acid

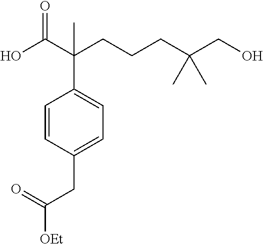 [SMILES: CCOC(=O)Cc1ccc(C(C)(CCCC(C)(C)CO)C(=O)O)cc1]

To a stirred solution of 2-(trimethylsilyl)ethyl 7-((tert-butyldimethylsilyl)oxy)-2-(4-(2-ethoxy-2-oxoethyl)phenyl)-2,6,6-trimethylheptanoate (Intermediate 53D, 8.6 g, 15.2 mmol) in tetrahydrofuran (9 mL) was added tetrabutylammonium fluoride (1 M in tetrahydrofuran, 122 mL, 122 mmol). The mixture was stirred at room temperature for 18 hours, quenched with water (50 mL) was added and extracted with ethyl acetate (3×150 mL). The combined ethyl acetate layers were dried over sodium sulfate and concentrated to afford the crude title compound (4.2 g, 79%) as a colorless oil. MS (ESI): 351 m/z [M+H]⁺, retention time: 1.95 minutes, purity: >99% (214 nm) (LC-MS method 5).

### Intermediate 53A: 2-(Trimethylsilyl)ethyl 2-(4-bromophenyl)acetate

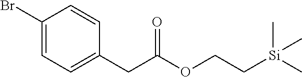 [SMILES: C[Si](C)(C)CCOC(=O)Cc1ccc(Br)cc1]

To a stirred solution of 2-(4-bromophenyl)acetic acid (33 g, 0.15 mol) in dichloromethane (300 mL) was added 2-(trimethylsilyl)ethanol (19.4 g, 0.16 mol), N,N′-dicyclohexylcarbodiimide (39 g, 0.19 mol), 4-dimethylaminopyridine (2.3 g, 0.02 mmol). The reaction was stirred at room temperature for 16 hours, then filtered. The filter cake was washed with dichloromethane (300 mL). The filtrate was concentrated. The residue was purified by silica gel column to afford 2-trimethylsilylethyl 2-(4-bromophenyl)acetate (40 g, 85%) as colorless oil. ¹H NMR (400 MHz, CDCl₃) δ 7.42 (d, J=8.3 Hz, 2H), 7.14 (d, J=8.3 Hz, 2H), 4.20-4.07 (m, 2H), 3.53 (s, 2H), 1.01-0.89 (m, 2H), −0.01 (s, 9H).

### Intermediate 53B: 2-(Trimethylsilyl)ethyl 2-(4-bromophenyl)propanoate

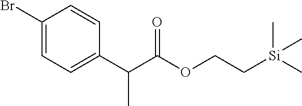 [SMILES: CC(C(=O)OCC[Si](C)(C)C)c1ccc(Br)cc1]

To a stirred and cooled (−78° C.) solution of 2-trimethylsilylethyl 2-(4-bromophenyl)acetate (Intermediate 53A, 42.5 g, 0.14 mol) in tetrahydrofuran (200 mL) was added lithium diisopropylamide (2 N in tetrahydrofuran, 100 mL, 0.20 mol). The mixture was stirred at −78° C. for 1 hour, then treated with methyl iodide (23 g, 0.16 mol), and stirred for an additional 16 hours at room temperature. The reaction mixture was quenched with saturated ammonium chloride (100 mL) and extracted with ethyl acetate (3×150 mL). The combined organic extracts were dried over sodium sulfate, and concentrated. The residue was purified by silica gel column chromatography to give the title compound (36 g, 81%) as an oil.

### Intermediate 53C: 2-(Trimethylsilyl)ethyl 2-(4-bromophenyl)-7-((tert-butyldimethylsilyl)oxy)-2,6,6-trimethylheptanoate

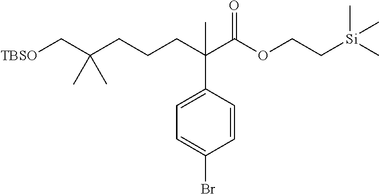 [SMILES: CC(C)(CCCC(C)(C(=O)OCC[Si](C)(C)C)c1ccc(Br)cc1)CO[Si](C)(C)C(C)(C)C]

To a stirred and cooled (−78° C.) solution of 2-(trimethylsilyl)ethyl 2-(4-bromophenyl)propanoate (Intermediate 53B, 18.1 g, 0.055 mol) in tetrahydrofuran (120 mL) was added lithium bis(trimethylsilyl)amide (2 M in tetrahydrofuran, 41 mL, 0.082 mol). The mixture was stirred at this temperature for 1 hour, then treated with tert-butyl((5-iodo-2,2-dimethylpentyl)oxy)dimethylsilane (44.8 g, 0.12 mol), and stirred for an additional 16 hours at room temperature. The reaction mixture was quenched with saturated ammonium chloride (30 mL) and extracted with ethyl acetate (3×150 mL). The combined ethyl acetate extracts were dried over sodium sulfate, and concentrated. The residue was purified by silica gel column chromatography to give the title compound (14 g, 46%) as an oil.

### Intermediate 53D: 2-(Trimethylsilyl)ethyl 7-((tert-butyldimethylsilyl)oxy)-2-(4-(2-ethoxy-2-oxoethyl)phenyl)-2,6,6-trimethylheptanoate

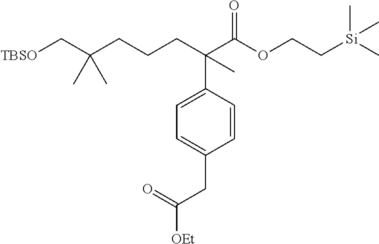 [SMILES: CCOC(=O)Cc1ccc(C(C)(CCCC(C)(C)CO[Si](C)(C)C(C)(C)C)C(=O)OCC[Si](C)(C)C)cc1]

To a stirred solution of 2-(trimethylsilyl)ethyl 2-(4-bromophenyl)-7-((tert-butyldimethylsilyl)oxy)-2,6,6-trimethylheptanoate (7.7 g, 13.8 mmol) in N,N-dimethylformamide (40 mL) was added ethyl 2-tributylstannylacetate (10.4 g, 27.6 mmol), dichlorobis(tri-o-tolylphosphine) palladium(II) (1.1 g, 1.38 mmol), zinc bromide (6.21 g, 27.6 mmol). The mixture was stirred at 100° C. for 5 hours, cooled to room temperature, and quenched with water (100 mL). The solution was extracted with ethyl acetate (3×100 mL). The combined organic extracts were washed with water, brine, dried over sodium sulfate, and concentrated. The residue was purified by silica gel column chromatography to give the title compound (5 g, 64%) as a yellow oil. The following intermediate was prepared based on the procedures described for Intermediate 53D.

| | | | |
|---|---|---|---|
| 53D-1 | 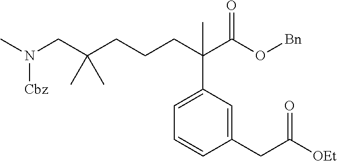 [SMILES: CCOC(=O)Cc1cccc(C(C)(CCCC(C)(C)CN(C)C(=O)OCc2ccccc2)C(=O)OCc2ccccc2)c1] | benzyl 7- (((benzyloxy)carbonyl)(methyl) amino)-2-(3-(2-ethoxy-2- oxoethyl)phenyl)-2,6,6- trimethylheptanoate | 610 [M + Na]⁺ |

### Intermediate 54: Benzyl 2-(4-(2-ethoxy-2-oxoethyl)phenyl)-7-hydroxy-2,6,6-trimethylheptanoate

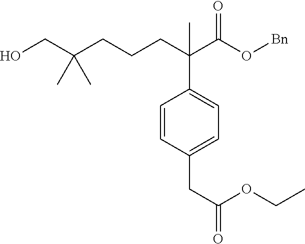 [SMILES: CCOC(=O)Cc1ccc(C(C)(CCCC(C)(C)CO)C(=O)OCc2ccccc2)cc1]

To a stirred solution of 2-(4-(2-ethoxy-2-oxoethyl)phenyl)-7-hydroxy-2,6,6-trimethylheptanoic acid (Intermediate 53, 4.7 g, 13.4 mmol) in acetone (60 mL) was added potassium carbonate (3.71 g, 26.8 mmol) and benzyl bromide (2.75 g, 16.1 mmol). The reaction mixture was stirred at 70° C. for 3 hours and concentrated. The residue was partitioned between water (50 mL) and ethyl acetate (50 mL). The separated organic layer, combined with two additional ethyl acetate (2×50 mL) extracts, was dried over sodium sulfate, and concentrate. The crude product was purified by silica gel column chromatography to afford the title compound (5.1 g, 86%) as a colorless oil. ¹H NMR (400 MHz, CDCl₃) δ 7.34-7.27 (m, 3H), 7.25-7.18 (m, 6H), 5.13-5.07 (m, 2H), 4.20-4.07 (m, 2H), 3.59 (s, 2H), 3.24-3.20 (m, 2H), 2.06-2.00 (m, 1H), 1.89-1.81 (m, 1H), 1.55 (s, 3H), 1.30-1.13 (m, 7H), 1.11-1.07 (m, 1H), 0.79-0.77 (m, 6H).

The following intermediate was prepared based on the procedures described for Intermediate 54.

| Inter. | | | MS m/z |
|---|---|---|---|
| No. | Structure | Name | [M + H]⁺ |
| 54-1 | 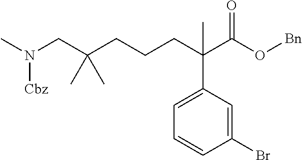 [SMILES: CN(CC(C)(C)CCCC(C)(C(=O)OCc1ccccc1)c1cccc(Br)c1)C(=O)OCc1ccccc1] | benzyl 7- (((benzyloxy)carbonyl)(methyl) amino)-2-(3- bromophenyl)-2,6,6- trimethylheptanoate | 602, 604 [M + Na]⁺ |
| 54-2 | 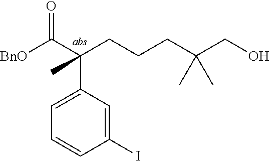 [SMILES: CC(C)(CO)CCC[C@@](C)(C(=O)OCc1ccccc1)c1cccc(I)c1] | benzyl (R)-7-hydroxy-2-(3- iodophenyl)-2,6,6- trimethylheptanoate | 503 [M + Na]⁺ |

### Intermediate 55: 7-(((benzyloxy)carbonyl)(methyl)amino)-2-(4-(2-ethoxy-2-oxoethyl)phenyl)-2,6,6-trimethylheptanoic acid

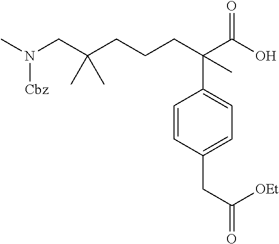 [SMILES: CCOC(=O)Cc1ccc(C(C)(CCCC(C)(C)CN(C)C(=O)OCc2ccccc2)C(=O)O)cc1]

To a stirred solution of benzyl 7-(((benzyloxy)carbonyl)(methyl)amino)-2-(4-(2-ethoxy-2-oxoethyl)phenyl)-2,6,6-trimethylheptanoate (Intermediate 31-5, 5.4 g, 9.2 mmol) in ethyl acetate (30 mL) and methanol (15 mL) was added palladium on carbon (0.5 g, 10%, 50% wet) was added. The reaction mixture was stirred at room temperature for 3 hours under hydrogen balloon, then filtered through a pad of Celite. The filtrate was concentrated. The residue (3.8 g) was dissolved in dichloromethane, treated with triethylamine (3.17 g, 31 mmol) and benzyl chloroformate (1.8 g, 10.6 mmol). The reaction was stirred at room temperature for 1 hour, quenched with water (15 ml). The solution was extracted with ethyl acetate (3×50 mL). The combined organic extracts were dried over sodium sulfate and concentrated. The residue was purified by silica gel column chromatography to afford the title compound (2.6 g, 60%) as a colorless oil. MS (ESI): 498 m/z [M+H]⁺, retention time: 2.23 minutes, purity: 97% (214 nm) (LC-MS method 5).

The following intermediate was prepared based on the procedures described for Intermediate 55.

| Inter. | | | MS m/z |
|---|---|---|---|
| No. | Structure | Name | [M + H]⁺ |
| 55-1 | 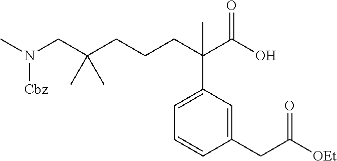 [SMILES: CCOC(=O)Cc1cccc(C(C)(CCCC(C)(C)CN(C)C(=O)OCc2ccccc2)C(=O)O)c1] | 7- (((benzyloxy)carbonyl)(methyl) amino)-2-(3-(2-ethoxy-2- oxoethyl)phenyl)-2,6,6- trimethylheptanoic acid | 498 |

### Intermediate 56: 7-(3-Bromophenyl)-8-hydrazineyl-3,3,7-trimethyl-8-oxooctanoic acid

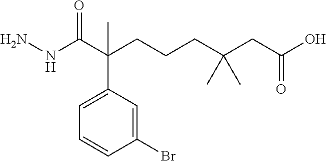 [SMILES: CC(C)(CCCC(C)(C(=O)NN)c1cccc(Br)c1)CC(=O)O]

To a stirred solution of 7-(3-bromophenyl)-8-methoxy-3,3,7-trimethyl-8-oxooctanoic acid (Intermediate 56C, 8.86 g, 23 mmol) in ethanol (40 mL) and hydrazine (80% in water) (80 mL) was heated to 110° C. for 12 hours and concentrated. The residue was purified by Pre-HPLC to give the title compound (4.2 g, 48%) as a white solid. MS (ESI): 385 m/z [M+H]⁺, retention time: 1.77 minutes, purity: 98% (214 nm) (LC-MS method 5).

### Intermediate 56A: Methyl 2-(3-bromophenyl)-2,6,6-trimethyloct-7-enoate

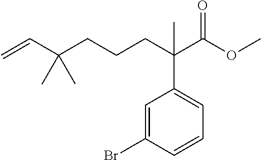 [SMILES: C=CC(C)(C)CCCC(C)(C(=O)OC)c1cccc(Br)c1]

To a stirred and cooled (0° C.) solution of methyltriphenylphosphonium bromide (15.4 g, 43.1 mmol) in tetrahydrofuran (200 mL) was added lithium bis(trimethylsilyl)amide (2M in tetrahydrofuran, 21.6 ml, 43.1 mmol). The mixture was stirred at 0° C. for 30 minutes, then treated with methyl 2-(3-bromophenyl)-2,6,6-trimethyl-7-oxoheptanoate (Intermediate 31B-6, 12.6 g, 28.7 mmol) and stirred at room temperature for additional 2 hours. The mixture was quenched with water (200 mL), extracted with ethyl acetate (3×200 mL). The combined organic layers were washed with brine, dried over sodium sulfate, and concentrated. The residue was purified with automated flash chromatography (eluting with ethyl acetate/petroleum ether=1/20 to afford the title compound (10 g, 91%) as yellow oil. MS (ESI): 375, 377 m/z [M+Na]⁺, retention time: 2.58 minutes, purity: 92% (214 nm) (LC-MS method 5),

### Intermediate 56B: Methyl 2-(3-bromophenyl)-8-hydroxy-2,6,6-trimethyloctanoate

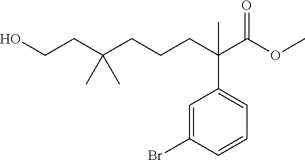 [SMILES: COC(=O)C(C)(CCCC(C)(C)CCO)c1cccc(Br)c1]

To a stirred and cooled (0° C.) solution of methyl 2-(3-bromophenyl)-2,6,6-trimethyloct-7-enoate (Intermediate 56A, 14 g, 36.4 mmol) in tetrahydrofuran (150 ml) was added borane-tetrahydrofuran complex (109 mL, 109 mmol, 1 M in tetrahydrofuran) drop wise. The reaction mixture was stirred at 0° C. for 4 hours, treated with sodium hydroxide (4.37 g, 109 mmol, in 36 mL of water), and stirred at room temperature for an additional 2 hours. The solution was partitioned between water (50 mL) and ethyl acetate (100 ml). The separated organic layer, combined with two additional ethyl acetate extracts (2×100 mL), was washed with water, brine, dried over sodium sulfate and concentrated. The residue was purified by automated flash chromatography (eluted with ethyl acetate in petroleum ether=0-20%) to afford the title compound (9.5 g, 70%) as colorless oil. MS (ESI): 371, 373 m/z [M+H]⁺, retention time: 2.44 minutes, purity: 95% (214 nm) (LC-MS method 5).

### Intermediate 56C: 7-(3-Bromophenyl)-8-methoxy-3,3,7-trimethyl-8-oxooctanoic acid

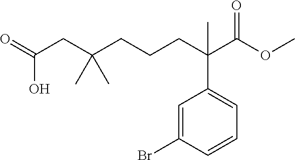 [SMILES: COC(=O)C(C)(CCCC(C)(C)CC(=O)O)c1cccc(Br)c1]

To a stirred solution of methyl 2-(3-bromophenyl)-8-hydroxy-2,6,6-trimethyloctanoate (Intermediate 56B, 8.5 g, 22.9 mmol) in t-butanol (200 mL) and a buffer solution of pH=4 (100 mL) was added (2,2,6,6-tetramethylpiperidin-1-yl)oxyl (TEMPO, 1.79 g, 11.4 mmol), sodium hypochlorite (0.852 g, 11.4 mmol), sodium chlorite (11.4 g, 126 mmol). The reaction mixture was stirred at room temperature overnight, quenched with water (100 mL), and extracted with ethyl acetate (3×100 mL). The combined organic phases were washed with water, brine, dried over sodium sulfate, and concentrated. The residue was purified by automated flash chromatography (eluting with ethyl acetate in petroleum ether=0-50%) to afford the title compound (8 g, 84%) as colorless oil. MS (ESI): 407 m/z [M+Na]⁺, retention time: 2.19 minutes, purity: 92% (214 nm) (LC-MS method 5).

The following intermediate was prepared based on the procedures described for Intermediate 56C.

| Inter. | | | MS m/z |
|---|---|---|---|
| No. | Structure | Name | [M + H]⁺ |
| 56C-1 | 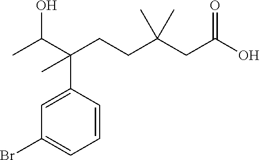 [SMILES: CC(O)C(C)(CCC(C)(C)CC(=O)O)c1cccc(Br)c1] | 6-(3-bromophenyl)-7- hydroxy-3,3,6- trimethyloctanoic acid | 339, 341 [M − H2O + H]⁺ |

### Intermediate 57: 3-(3-(3-Bromophenyl)-4-hydroxy-3-methylbutoxy)-2,2-dimethylpropanenitrile

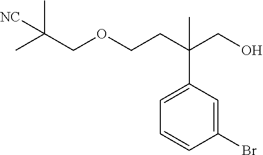 [SMILES: CC(C)(C#N)COCCC(C)(CO)c1cccc(Br)c1]

To a stirred solution of methyl 2-(3-bromophenyl)-4-(2-cyano-2-methylpropoxy)-2-methylbutanoate (Intermediate 19A-26, 5.60 g, 0.0152 mol) in tetrahydrofuran (70 mL), methanol (3.5 mL) was added lithium borohydride (1.33 g, 0.0605 mol) in four portions at 1-hour intervals. The mixture was stirred at room temperature for 16 hours, quenched with 20 mL of saturated ammonium chloride, and concentrated to remove methanol. The aqueous residue was diluted with water (100 mL) and extracted with ethyl acetate (3×200 mL). The combined organic layers were washed with brine, dried over sodium sulfate, and concentrated. The residue was purified by flash chromatography (eluting with petroleum ether: ethyl acetate=4:1) to afford the title compound (4.84 g, 90%) as a colorless oil. MS (ESI): 362, 364 m/z [M+Na]⁺, retention time: 2.09 minutes, purity: 97% (214 nm) (LC-MS method 7). ¹H NMR (400 MHz, CDCl₃): δ 7.50 (s, 1H), 7.36 (d, J=7.6 Hz, 1H), 7.32 (d, J=8.0 Hz, 1H), 7.22 (t, J=8.0 Hz, 1H), 3.71 (d, J=6.8 Hz, 2H), 3.48-3.43 (m, 2H), 3.29 (s, 2H), 2.03-1.96 (m, 2H), 1.35 (s, 3H), 1.31 (s, 6H).

### Intermediate 58: 2-(3-Bromophenyl)-7-hydroxy-2,5,5-trimethylheptanal

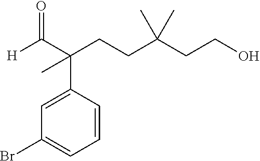 [SMILES: CC(C)(CCO)CCC(C)(C=O)c1cccc(Br)c1]

To a stirred and cooled (0° C.) solution of 2-(3-bromophenyl)-7-hydroxy-N-methoxy-N,2,5,5-tetramethyl-heptanamide (Intermediate 11A-4, 400 mg, 1 mmol) in tetrahydrofuran was added diisobutylaluminum hydride (DIBAL-H, 1M in tetrahydrofuran, 4 ml, 4 mmol). The reaction mixture was stirred at 0° C. for 1 hour, quenched with saturated ammonium chloride, and extracted with ethyl acetate (3×50 mL). The combined organic extracts were washed with aqueous hydrochloric acid, saturated sodium bicarbonate, brine, dried over sodium sulfate, and concentrated. The crude title compound (370 mg) was obtained as a colorless oil and used for next step without further purification. MS (ESI): 327, 329 m/z [M+H]⁺, retention time: 2.06 minutes, purity: >99% (254 nm) (LC-MS method 4).

### Intermediate 59: 5-((4-Bromo-6,7-difluoro-1H-indol-5-yl)oxy)-2-fluorobenzonitrile

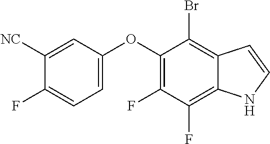 [SMILES: N#Cc1cc(Oc2c(F)c(F)c3[nH]ccc3c2Br)ccc1F]

To a stirred solution of 5-(4-amino-2-bromo-5,6-difluoro-3-((trimethylsilyl)ethynyl)phenoxy)-2-fluorobenzonitrile (Intermediate 59E, 700 mg, 1.6 mmol) in N,N-dimethylformamide (7 mL) was added copper (I) iodide (608 mg, 3.2 mmol). The mixture was stirred at 100° C. in glove box for four hours. The reaction mixture was diluted with water and extracted with ethyl acetate (3×50 mL). The organic combined phases were washed with water, brine, dried with sodium sulfate, and concentrated. The residue was purified by silica gel flash chromatography to give the title compound as a yellow solid (362 mg, 62%). ¹H NMR (400 MHz, CDCl₃) δ 8.63 (s, 1H), 7.37-7.39 (m, 1H), 7.14-7.23 (m, 2H), 7.04-7.06 (m, 1H), 6.67-6.69 (m, 1H) ppm.

### Intermediate 59A: 1-Bromo-2,3,4-trifluoro-5-nitrobenzene

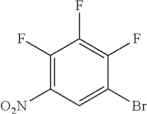 [SMILES: O=[N+]([O-])c1cc(Br)c(F)c(F)c1F]

To a solution of 1,2,3-trifluoro-4-nitrobenzene (30 g, 169.5 mmol) in concentrated sulfuric acid (150 mL) was added 1,3-dibromo-5,5-dimethylimidazolidine-2,4-dione (24 g, 84.7 mmol) at 0° C. and stirred at room temperature overnight. The mixture was slowly and carefully added to ice water (600 g ice and 100 mL water) to keep the temperature below 30° C. and extracted with heptane (300 mL×3). The combined organic extracts were washed with water and brine, dried over magnesium sulfate, filtered and evaporated to dryness. The resulting residue was purified by flash chromatography over silica (heptane) to give the title compound (32 g, 75%) as a yellow oil. ¹H NMR (400 MHz, CDCl₃) δ 8.21 (td, J=7.2, 2.8 Hz, 1H) ppm.

### Intermediate 59B: 5-(6-Bromo-2,3-difluoro-4-nitrophenoxy)-2-fluorobenzonitrile

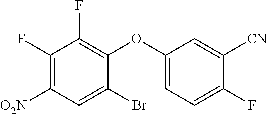 [SMILES: N#Cc1cc(Oc2c(Br)cc([N+](=O)[O-])c(F)c2F)ccc1F]

To a solution of 1-bromo-2,3,4-trifluoro-5-nitrobenzene (Intermediate 59A, 32 g, 125.5 mmol) in DMF (250 mL) were added 2-fluoro-5-hydroxybenzonitrile (18.9 g, 138.0 mmol) and potassium carbonate (26 g, 1.5 mmol) at room temperature and stirred for one hour. The reaction mixture was diluted with water (300 mL) and extracted with ethyl acetate (250 mL×3). The combined organic extracts were washed with water and brine, dried over magnesium sulfate, filtered and concentrated. The residue was purified by flash chromatography over silica to give the title compound as a yellow solid (15 g, 30%). MS: 373, 375 m/z [M+H]⁺. ¹H NMR (400 MHz, CDCl₃) δ 8.30 (dd, J=7.2, 2.4 Hz, 1H), 7.25-7.20 (m, 2H), 7.17-7.15 (m, 1H) ppm.

### Intermediate 59C: 5-(4-Amino-6-bromo-2,3-difluorophenoxy)-2-fluorobenzonitrile

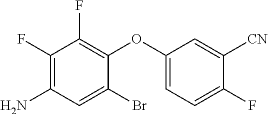 [SMILES: N#Cc1cc(Oc2c(Br)cc(N)c(F)c2F)ccc1F]

To a stirred solution of 5-(6-bromo-2,3-difluoro-4-nitrophenoxy)-2-fluorobenzonitrile (Intermediate 59B, 5 g, 13.4 mmol) in ethanol (100 mL) and water (30 mL) were added iron powder (3 g, 53.6 mmol) and ammonium chloride (5.8 g, 107.5 mmol). The reaction mixture was stirred at 80° C. for four hours, cooled to room temperature, diluted with water (100 mL) and extracted with ethyl acetate (3×100 mL). The combined organic extracts were washed with water, brine, dried over magnesium sulfate, and concentrated. The residue was purified by flash chromatography over silica (heptane/ethyl acetate, v/v, 10/1) to give the title compound as a yellow solid (3 g, 65%). MS: 343, 345 m/z [M+H]⁺.

### Intermediate 59D: 5-(4-Amino-2-bromo-5,6-difluoro-3-iodophenoxy)-2-fluorobenzonitrile

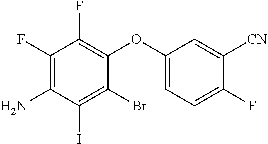 [SMILES: N#Cc1cc(Oc2c(F)c(F)c(N)c(I)c2Br)ccc1F]

To a solution of 5-(4-amino-6-bromo-2,3-difluorophenoxy)-2-fluorobenzonitrile (Intermediate 59C, 6.7 g, 19.6 mmol) in acetic acid (200 mL) was added NIS (4.4 g, 19.6 mmol). The mixture was stirred at room temperature for three hours, diluted with water (200 mL) and extracted with ethyl acetate (150 mL×3). The combined organic extracts were washed with water, brine, dried over magnesium sulfate, filtered and evaporated to dryness. The residue was purified by flash chromatography over silica (petroleum ether/dichloromethane, v/v, 2/1) to give the title compound as a yellow solid (8.2 g, 89%). ¹H NMR (400 MHz, CDCl₃) δ 7.18-7.16 (m, 2H), 7.04-7.03 (m, 1H), 4.56 (s, 2H) ppm. MS: 469, 471 m/z [M+H]⁺.

### Intermediate 59E: 5-(4-Amino-2-bromo-5,6-difluoro-3-((trimethylsilyl)ethynyl)phenoxy)-2-fluorobenzonitrile

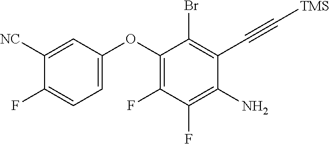 [SMILES: C[Si](C)(C)C#Cc1c(N)c(F)c(F)c(Oc2ccc(F)c(C#N)c2)c1Br]

To a solution of 5-(4-amino-2-bromo-5,6-difluoro-3-iodophenoxy)-2-fluorobenzonitrile (Intermediate 59D, 8.1 g, 17.3 mmol) in DMF (200 mL) were added trimethylsilylacetylene (3.4 g, 34.6 mmol), Pd(Ph₃P)₂Cl₂ (1.2 g, 1.7 mmol), CuI (323 mg, 1.7 mmol) and triethylamine (3.5 g, 34.6 mmol). The reaction mixture was stirred at 30° C. under nitrogen for three hours, diluted with water (300 mL) and extracted with ethyl acetate (3×250 mL). The combined organic extracts were washed with water, brine, dried over sodium sulfate, and concentrated. The residue was purified by flash chromatography over silica (petroleum ether/dichloromethane, v/v, 8/1) to give the title compound as a yellow solid (5 g, 64%). MS: 439, 441 m/z [M+H]⁺.

### Intermediate 60: Ethyl 2-(6-bromo-4-(3-bromophenyl)-4-methyl-5-oxohexyl)cyclopropane-1-carboxylate

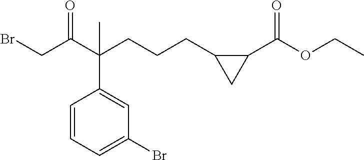 [SMILES: CCOC(=O)C1CC1CCCC(C)(C(=O)CBr)c1cccc(Br)c1]

To a stirred and cooled (0° C.) solution of 2-(3-bromophenyl)-5-(2-(ethoxycarbonyl)cyclopropyl)-2-methylpentanoic acid (Intermediate 60C, 1 g, 2.61 mmol) in 10 mL of dichloromethane was added oxalyl chloride (662 mg, 5.22 mmol). The mixture was stirred for 30 minutes at this temperature and concentrated. The residue was diluted with 20 mL of heptanes and concentrated. This process was repeated once more to remove any trace of oxalyl chloride. The mixture was dissolved in 10 mL of acetonitrile, cooled to 0° C., and treated with (trimethylsilyl)diazomethane (5.22 mL, 10.4 mmol) dropwise. The solution was slowly allowed to warm to room temperature and stirred for 12 hours and concentrated. The residue was dissolved in 10 mL of dichloromethane, cooled to 0° C., and treated with hydrogen bromide (in acetic acid, 844 mg, 10.4 mmol) dropwise. The mixture was stirred for 30 minutes until all the gas bubble stopped. The solvent was evaporated, and the residue was purified by column chromatography (eluting with 0-20% ethyl acetate in heptanes to give title compound (700 mg, 55.4%) as an oil. MS (ESI): 483 m/z [M+Na]⁺, retention time: 2.34 minutes, purity: 88% (214 nm) (LC-MS method 20).

### Intermediate 60A: 1-Ethyl 8-(2-(trimethylsilyl)ethyl) (Z)-7-(3-bromophenyl)-7-methyloct-2-enedioate

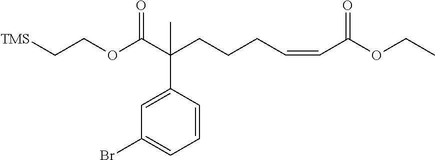 [SMILES: CCOC(=O)/C=C\CCCC(C)(C(=O)OCC[Si](C)(C)C)c1cccc(Br)c1]

To a stirred solution of 2-(trimethylsilyl)ethyl 2-(3-bromophenyl)-2-methylhept-6-enoate (Intermediate 19A-29, 10 g, 25.2 mmol) and ethyl acrylate (15.1 g, 151 mmol) in 150 ml of 1,2-dichloroethane was added Grubbs catalyst 2nd generation (3.2 g, 3.77 mmol). The mixture was refluxed for 24 hours, cooled to room temperature, and quenched with brine. The solution was extracted with ethyl acetate (3×100 mL). The combined organic phases were dried over sodium sulfate and concentrated. The residue was purified by silica gel column chromatography (eluting with 40% dichloromethane in petroleum ether) to afford the title compound (10 g, 76%) as a colorless oil.

### Intermediate 60B: Ethyl 2-(4-(3-bromophenyl)-4-methyl-5-oxo-5-(2-(trimethylsilyl)ethoxy)pentyl)cyclopropane-1-carboxylate

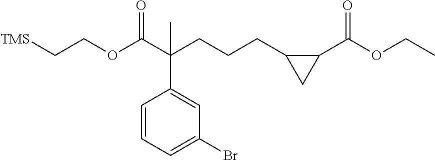 [SMILES: CCOC(=O)C1CC1CCCC(C)(C(=O)OCC[Si](C)(C)C)c1cccc(Br)c1]

To a stirred solution of trimethylsulfoxonium iodide (3.38 g, 15.3 mmol) in dimethyl sulfoxide (40 mL) was added potassium tert-butoxide (1.58 g, 14.1 mmol). The mixture was stirred for 2 hours, then treated with a solution of 1-ethyl 8-(2-(trimethylsilyl)ethyl) (Z)-7-(3-bromophenyl)-7-methyloct-2-enedioate (Intermediate 60A, 4 g, 8.52 mmol) in dimethyl sulfoxide (5 mL) dropwise. The reaction was stirred overnight, then quenched with water (200 ml), and extracted with ethyl acetate (2×200 mL). The combined organic layers were washed with brine, dried over sodium sulfate, and concentrated. The crude title compound (3.8 g, 55%) was used for next step without further purification. MS (ESI): 505 m/z [M+Na]⁺, retention time: 2.66 minutes, purity: 50% (254 nm) (LC-MS method 20).

### Intermediate 60C: 2-(3-Bromophenyl)-5-(2-(ethoxycarbonyl)cyclopropyl)-2-methylpentanoic acid

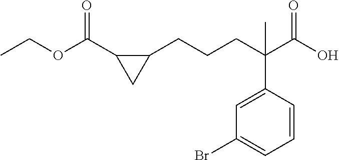 [SMILES: CCOC(=O)C1CC1CCCC(C)(C(=O)O)c1cccc(Br)c1]

A mixture of ethyl 2-(4-(3-bromophenyl)-4-methyl-5-oxo-5-(2-(trimethylsilyl)ethoxy)pentyl)cyclopropane-1-carboxylate (Intermediate 60B, 3.8 g, 4.72 mmol) and tetra-n-butyl ammonium fluoride solution (1 M in tetrahydrofuran, 15 mL, 15 mmol) was stirred at room temperature for 2 hours, then quenched with water (200 ml), and extracted with ethyl acetate (2×200 mL). The combined organic extracts were washed with brine, dried over sodium sulfate, and concentrated. The residue was purified by silica gel column chromatography (eluting with 20% methanol in dichloromethane) to afford the title compound (1.6 g, 80%) as a colorless oil.

The following intermediate was prepared based on the procedures described for Intermediate 60C.

| Inter. | | | MS m/z |
|---|---|---|---|
| No. | Structure | Name | [M + H]⁺ |
| 60C-1 | 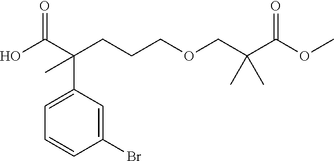 [SMILES: COC(=O)C(C)(C)COCCCC(C)(C(=O)O)c1cccc(Br)c1] | 2-(3-bromophenyl)-5-(3- methoxy-2,2-dimethyl-3- oxopropoxy)-2- methylpentanoic acid | 423, 425 [M + Na]⁺ |

### Intermediate 61: 2-Fluoro-5-((6-fluoro-4-((2-oxooxazolidin-5-yl)methyl)-1H-indol-5-yl)oxy)benzimidamide

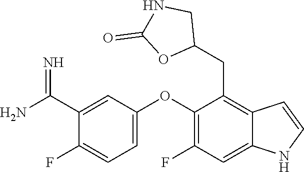 [SMILES: N=C(N)c1cc(Oc2c(F)cc3[nH]ccc3c2CC2CNC(=O)O2)ccc1F]

To a stirred and cooled (0° C.) solution of benzyl (3-(5-(3-cyano-4-fluorophenoxy)-6-fluoro-1H-indol-4-yl)-2-hydroxypropyl)carbamate (Intermediate 61A, 4.6 g, 9.63 mmol) in tetrahydrofuran (5 mL) was added lithium bis(trimethylsilyl)amide (96.3 mL, 96.3 mmol, 1M in tetrahydrofuran). The reaction mixture was stirred at room temperature for 12 hours, then quenched with water (200 ml), and extracted with ethyl acetate (2×200 mL). The combined organic extracts were washed with brine, dried over sodium sulfate, and concentrated. The residue was purified by silica gel column chromatography (eluting with 40% methanol in dichloromethane) to afford the title compound (2.2 g, 56%) as a solid. MS (ESI): 387 m/z [M+H]⁺, retention time: 1.18 minutes, purity: 80% (214 nm) (LC-MS method 20).

### Intermediate 61A: Benzyl (3-(5-(3-cyano-4-fluorophenoxy)-6-fluoro-1H-indol-4-yl)-2-hydroxypropyl)carbamate

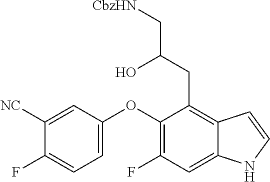 [SMILES: N#Cc1cc(Oc2c(F)cc3[nH]ccc3c2CC(O)CNC(=O)OCc2ccccc2)ccc1F]

To a stirred solution of benzyl ((4-chlorobenzoyl)oxy)carbamate (7.8 g, 25.5 mmol) in acetonitrile (60 mL) and water (6 mL) was added osmium tetroxide (389 mg, 1.53 mmol). The mixture was stirred at room temperature for 10 minutes, then treated with 5-((4-allyl-6-fluoro-1H-indol-5-yl)oxy)-2-fluorobenzonitrile (Intermediate 12, 6.2 g, 20 mmol). The reaction mixture was stirred at room temperature for 12 hours, quenched with water (100 ml) and extracted with ethyl acetate (2×100 mL). The combined organic extracts were washed with brine, dried over sodium sulfate, and concentrated. The crude was purified by silica gel column chromatography (eluting with 50% ethyl acetate in petroleum ether) to afford the title compound (7 g, 54%). MS (ESI): 478 m/z [M+H]⁺, retention time: 2.02 minutes, purity: 80% (214 nm) (LC-MS method 20).

### Intermediate 62: Methyl 3-(3-iodopropoxy)-2,2-dimethylpropanoate

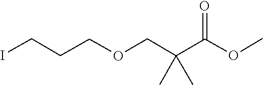

To a stirred solution of methyl 2,2-dimethyl-3-(3-(tosyloxy)propoxy)propanoate (Intermediate 62C, 18.4 g, 53.5 mmol) in acetone (400 mL) was added sodium iodide (24.1 g, 161 mmol). The mixture was stirred for 16 hours at 65° C., cooled to room temperature, and diluted with water (600 mL). The solution was extracted with ethyl acetate (2×275 mL). The combined organic extracts were washed with brine (160 mL), dried over sodium sulfate, and concentrated. The residue was purified by automated column chromatography (120 g silica gel column, eluting with ethyl acetate in petroleum ether from 0 to 12%) to give the title compound (11.8 g; 63%) as an oil. MS (ESI): 301 m/z [M+H]⁺, purity: 96% (254 nm) (LC-MS method 5).

### Intermediate 62A: Methyl 3-(3-(benzyloxy)propoxy)-2,2-dimethylpropanoate

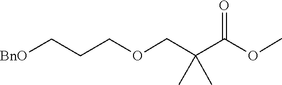 [SMILES: COC(=O)C(C)(C)COCCCOCc1ccccc1]

To a stirred and cooled (0° C.) solution of methyl 3-hydroxy-2,2-dimethyl-propanoate (9.24 g, 70 mmol) and 3-(benzyloxy)propyl 4-methylbenzenesulfonate (20 g, 62.4 mmol) in N,N-dimethylformamide (240 mL) was added sodium hydride (60 weight % in mineral oil, 3.74 g, 93.6 mmol). The reaction mixture was stirred at room temperature for 2 hours, then quenched with saturated ammonium chloride (100 mL), diluted with water (800 mL), and extracted with ethyl acetate (3×250 mL). The combined organic layers were dried over sodium sulfate and concentrated. The residue was purified by automated flash chromatography (300 g silica gel column, eluting with 0-10% ethyl acetate in petroleum ether) to give the title compound (11.9 g, 37%) as a colorless oil. MS (ESI): 281 m/z [M+H]⁺, retention time: 2.30 minutes, purity: 54% (254 nm) (LC-MS method 5).

### Intermediate 62B: Methyl 3-(3-hydroxypropoxy)-2,2-dimethylpropanoate

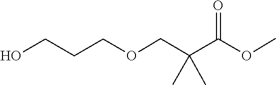

To a solution of methyl 3-(3-(benzyloxy)propoxy)-2,2-dimethylpropanoate (Intermediate 62A, 19.5 g; 69.6 mmol) in methanol (300 mL) was added 10% Pd on carbon (50% wet, 3.6 g). The mixture was stirred under hydrogen balloon at room temperature for 16 hours and filtered through a pad of Celite. The filtrate was concentrated to give the crude title compound (12.2 g, 92%) as an oil.

### Intermediate 62C: Methyl 2,2-dimethyl-3-(3-(tosyloxy)propoxy)propanoate

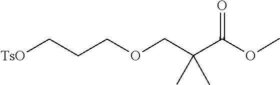 [SMILES: COC(=O)C(C)(C)COCCCOS(=O)(=O)c1ccc(C)cc1]

To a stirred and cooled (0° C.) solution of methyl 3-(3-hydroxypropoxy)-2,2-dimethylpropanoate (Intermediate 62B, 12 g; 63.1 mmol) in tetrahydrofuran (350 mL) was added tosyl chloride (18 g, 94.6 mmol), triethylamine (13.2 g, 94.6 mmol) and 4-N,N-dimethylaminopyridine (0.77 g, 6.3 mmol). The mixture was stirred at room temperature for 16 hours, quenched with water (350 mL), and extracted with dichloromethane (2×150 mL). The combined organic phases were dried over anhydrous sodium sulfate and concentrated. The residue was purified by automated column chromatography (120 g silica gel column, eluting with 0-15% ethyl acetate in petroleum ether) to give the title compound (19 g, 70%) as a colorless oil. MS (ESI): 345 m/z [M+H]⁺, retention time: 2.22 minutes, purity: 80% (254 nm) (LC-MS method 5).

### Intermediate 63: Methyl 4-(2-bromo-2-(3-bromophenyl)ethoxy)-2,2-dimethylbutanoate

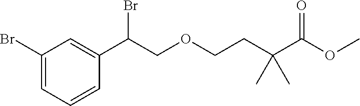 [SMILES: COC(=O)C(C)(C)CCOCC(Br)c1cccc(Br)c1]

To a stirred and cooled (0° C.) solution of methyl 4-(2-(3-bromophenyl)-2-hydroxyethoxy)-2,2-dimethylbutanoate (Intermediate 63A, 6.1 g, 17.7 mmol) in dichloromethane (150 mL) was added triphenylphosphine (13.9 g, 53 mmol) and N-bromosuccinimide (9.43 g, 53 mmol). The mixture was stirred at 0° C. for 3 hours and concentrated. The residue was purified by automated flash chromatography (eluting with ethyl acetate in petroleum ether from 0 to 10%) to give the title compound (6.1 g, 78%) as light-yellow oil. MS (ESI): 409 m/z [M+H]⁺, retention time: 2.30 minutes, purity: 92% (214 nm) (LC-MS method 22).

### Intermediate 63A: Methyl 4-(2-(3-bromophenyl)-2-hydroxyethoxy)-2,2-dimethylbutanoate

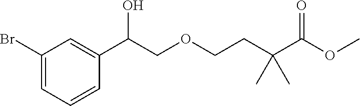 [SMILES: COC(=O)C(C)(C)CCOCC(O)c1cccc(Br)c1]

To a stirred and cooled (−40° C.) solution of 1-bromo-3-iodo-benzene (13.4 g, 47.3 mmol) in tetrahydrofuran (150 mL) was added isopropyl magnesium chloride (1 M in tetrahydrofuran, 47.3 mL) dropwise. The mixture was stirred at −40° C. for 1 hour, then treated with a solution of methyl 2,2-dimethyl-4-(2-oxoethoxy)butanoate (Intermediate 43-1, 8.9 g, 47.3 mmol) in 10 mL of tetrahydrofuran dropwise. The mixture was stirred at 0° C. for 1.5 hours, quenched with 200 mL of saturated ammonium chloride, and extracted with ethyl acetate (2×200 mL). The combined organic phases were dried over sodium sulfate and concentrated. The residue was purified by silica gel column chromatography (eluting with ethyl acetate in petroleum ether from 0 to 20%) to give the title compound (7.2 g, 42%) as light-yellow oil. MS (ESI): 345, 347 m/z [M+H]⁺, retention time: 2.10 minutes, purity: 97% (214 nm) (LC-MS method 22).

### Intermediate 64: Ethyl 3-(5-(3-cyano-4-fluorophenoxy)-6-fluoro-1H-indol-4-yl)propanoate

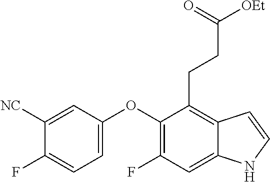 [SMILES: CCOC(=O)CCc1c(Oc2ccc(F)c(C#N)c2)c(F)cc2[nH]ccc12]

To a stirred solution of ethyl (E)-3-(5-(3-cyano-4-fluorophenoxy)-6-fluoro-1H-indol-4-yl)acrylate (Intermediate 4, 6.00 g, 21.7 mmol) in 150 mL of ethyl acetate was added 2 g of palladium on carbon (10%, 50% wet). The reaction was stirred at 40° C. under hydrogen balloon overnight. The mixture was filtered through a pad of Celite. The filtrate was concentrated to afford the title compound (4.51 g, 75%) as a yellow oil. ¹H NMR (400 MHz, CDCl₃) δ 8.34 (s, 1H), 7.28-7.26 (m, 1H), 7.17-7.11 (m, 3H), 7.00 (dd, J=5.2, 3.2 Hz, 1H), 6.63-6.61 (m, 1H), 4.09 (q, J=6.8 Hz, 2H), 3.14 (t, J=8.0 Hz, 2H), 2.60 (t, J=8.0 Hz, 2H), 1.20 (t, J=6.8 Hz, 3H) ppm.

### Intermediate 65: Ethyl 3-(6-fluoro-5-(4-fluoro-3-(imino(methylthio)methyl)phenoxy)-1H-indol-4-yl)propanoate hydroiodide

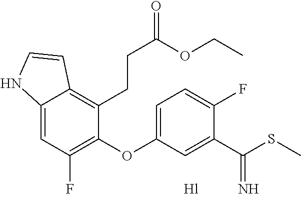 [SMILES: CCOC(=O)CCc1c(Oc2ccc(F)c(C(=N)SC)c2)c(F)cc2[nH]ccc12]

To a stirred solution of ethyl 3-(5-(3-carbamothioyl-4-fluorophenoxy)-6-fluoro-1H-indol-4-yl)propanoate (Intermediate 65A, 1.80 g, 4.5 mmol) in 10 ml acetone was added iodomethane (3.16 g, 22.3 mmol). The reaction vial was sealed, and the mixture was stirred at room temperature overnight. The solution was concentrated to give the crude title compound (2.40 g) as an orange solid. This crude solid was used for next step without further purification. MS (ESI): 419 m/z [M+H]⁺, retention time: 1.76 minutes, purity: 80% (214 nm) (LC-MS method 5).

The following intermediates were prepared based on the procedures described for Intermediate 65.

| Inter. | | | MS m/z |
|---|---|---|---|
| No. | Structure | Name | [M + H]⁺ |
| 65-1 | 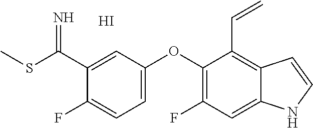 | methyl 2-fluoro-5-((6-fluoro- 4-vinyl-1H-indol-5- yl)oxy)benzimidothioate hydroiodide | 345 |
| 65-2 | 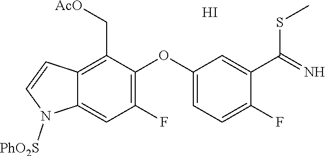 | (6-fluoro-5-(4-fluoro-3- (imino(methylthio)methyl) phenoxy)-1-(phenylsulfonyl)- 1H-indol-4-yl)methyl acetate hydroiodide | 531 |
| 65-3 | 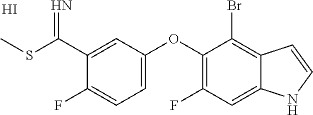 | methyl 5-((4-bromo-6-fluoro- 1H-indol-5-yl)oxy)-2- fluorobenzimidothioate hydroiodide | 397, 399 |

### Intermediate 65A: Ethyl 3-(5-(3-carbamothioyl-4-fluorophenoxy)-6-fluoro-1H-indol-4-yl)propanoate

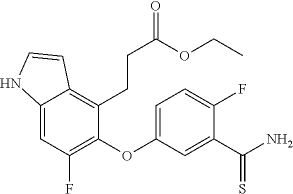 [SMILES: CCOC(=O)CCc1c(Oc2ccc(F)c(C(N)=S)c2)c(F)cc2[nH]ccc12]

To a stirred solution of ethyl 3-(5-(3-cyano-4-fluorophenoxy)-6-fluoro-1H-indol-4-yl)propanoate (Intermediate 64, 2.00 g, 5.4 mmol) in N,N-dimethylformamide (20 ml) was added magnesium chloride (1.54 g, 16.2 mmol), sodium hydrosulfide (1.82 g, 32.4 mmol) and water (1.2 mL, 64.8 mmol). The reaction was stirred at room temperature for 1 hour, quenched with water (50 mL), and extracted with ethyl acetate (3×40 ml). The combined organic layers were washed with brine, dried over sodium sulfate and concentrated. The residue was purified by silica gel column chromatography (eluting with petroleum ether: ethyl acetate=2:1) to give the title compound (1.80 g, 76%) as a yellow oil. MS (ESI): 405 m/z [M+H]⁺, retention time: 1.13 minutes, purity: 98% (214 nm) (LC-MS method 23).

The following intermediates were prepared based on the procedures described for Intermediate 65A.

| Inter. | | | MS m/z |
|---|---|---|---|
| No. | Structure | Name | [M + H]⁺ |
| 65A-1 | 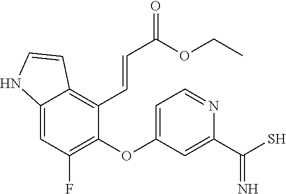 [SMILES: CCOC(=O)/C=C/c1c(Oc2ccnc(C(=N)S)c2)c(F)cc2[nH]ccc12] | (E)-4-((4-(3-ethoxy-3- oxoprop-1-en-1-yl)-6-fluoro- 1H-indol-5-yl)oxy)pyridine-2- carbimidothioic acid | 386 |
| 65A-2 | 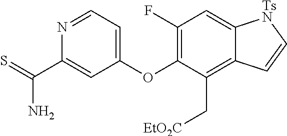 [SMILES: CCOC(=O)Cc1c(Oc2ccnc(C(N)=S)c2)c(F)cc2c1ccn2S(=O)(=O)c1ccc(C)cc1] | ethyl 2-(5-((2- carbamothioylpyridin-4- yl)oxy)-6-fluoro-1-tosyl-1H- indol-4-yl)acetate | 528 |

### Intermediate 66: Tert-Butyl(4-iodo-2,2-dimethylbutoxy)dimethylsilane

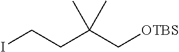

To a vigorously stirred solution of triphenylphosphine (20.98 g, 80 mmol) in 150 ml dichloromethane was added imidazole (5.44 g, 80 mmol) and iodine (20.30 g, 80 mmol). The reaction was stirred at 0° C. for 15 minutes, then treated with 4-((tert-butyldimethylsilyl)oxy)-3,3-dimethylbutan-1-ol (Intermediate 66A, 14.42 g, 62 mmol). The reaction was stirred at room temperature for 4 hours, diluted with 20% ethyl acetate in hexanes (500 mL) and filtered. The filtrate was washed with saturated sodium sulfite, dried over sodium sulfate, and concentrated. The residue was purified by silica gel column chromatography (petroleum ether/ethyl acetate=20/1) to give the title compound (9.5 g, 44%) as a yellow oil. ¹H NMR (400 MHz, CDCl₃) δ 3.21 (s, 2H), 3.18-3.13 (m, 2H), 1.95-1.90 (m, 2H), 0.87 (s, 9H), 0.82 (s, 6H), 0.00 (s, 6H) ppm.

### Intermediate 66A: 4-((Tert-Butyldimethylsilyl)oxy)-3,3-dimethylbutan-1-ol

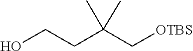

To a stirred solution of (4-(benzyloxy)-2,2-dimethylbutoxy)(tert-butyl)dimethylsilane (Intermediate 30C-1, 21.00 g, 65 mmol) in 200 ml of ethyl acetate was added 2 g of palladium on carbon (10%, 50% wet). The reaction was stirred at 35° C. under hydrogen balloon overnight, then filtered through a pad of Celite. The filtrate was concentrated to afford the title compound (14.42 g, 95%) as a colorless liquid. ¹H NMR (400 MHz, CDCl₃) δ 3.58-3.55 (m, 2H), 3.37 (br s, 1H), 3.27 (s, 2H), 1.48 (t, J=5.8 Hz, 2H), 0.84 (s, 9H), 0.81 (s, 6H), 0.00 (s, 6H) ppm.

### Intermediate 67: Methyl 7-(3-bromophenyl)-2,2-dimethyl-8-(2-methylhydrazineyl)-8-oxooctanoate

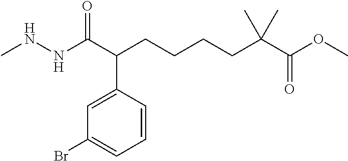

To a stirred solution of tert-butyl 2-(2-(3-bromophenyl)-8-methoxy-7,7-dimethyl-8-oxooctanoyl)-1-methylhydrazine-1-carboxylate (Intermediate 67A, 14.0 g, 0.028 mol) in dichloromethane (100 mL) was added trifluoroacetic acid (20 mL). The mixture was stirred at room temperature for two hours and concentrated. The residue was dissolved in dichloromethane (200 mL), washed with saturated sodium bicarbonate, brine, dried over sodium sulfate, and concentrated to give the title compound (10.4 g, 93%) as a light-yellow oil. MS (ESI): 399, 401 m/z [M+H]⁺, retention time: 1.12 minutes, purity: 97% (214 nm) (LC-MS method 14).

The following intermediates were prepared based on the procedures described for Intermediate 67.

| Inter. | | | MS m/z |
|---|---|---|---|
| No. | Structure | Name | [M + H]⁺ |
| 67-1 | 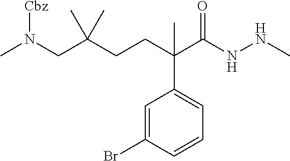 | benzyl (5-(3-bromophenyl)- 2,2,5-trimethyl-6-(2- methylhydrazineyl)-6- oxohexyl)(methyl)carbamate | 504, 506 |
| 67-2 | 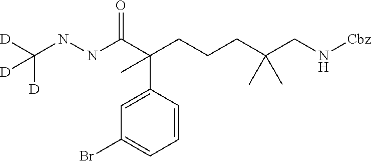 [SMILES: [2H]C([2H])([2H])NNC(=O)C(C)(CCCC(C)(C)CNC(=O)OCc1ccccc1)c1cccc(Br)c1] | benzyl (6-(3-bromophenyl)- 2,2,6-trimethyl-7-(2-(methyl- d3)hydrazineyl)-7- oxoheptyl)carbamate | 507, 509 |
| 67-3 | 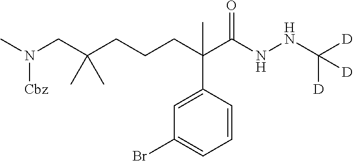 [SMILES: [2H]C([2H])([2H])NNC(=O)C(C)(CCCC(C)(C)CN(C)C(=O)OCc1ccccc1)c1cccc(Br)c1] | benzyl (6-(3-bromophenyl)- 2,2,6-trimethyl-7-(2-(methyl- d3)hydrazineyl)-7- oxoheptyl)(methyl)carbamate | 521, 523 |
| 67-4 | 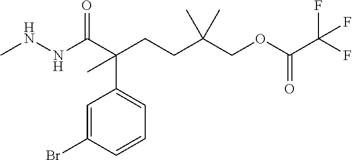 [SMILES: CNNC(=O)C(C)(CCC(C)(C)COC(=O)C(F)(F)F)c1cccc(Br)c1] | 5-(3-bromophenyl)-2,2,5- trimethyl-6-(2- methylhydrazineyl)-6- oxohexyl 2,2,2- trifluoroacetate | 453, 455 |
| 67-5 | 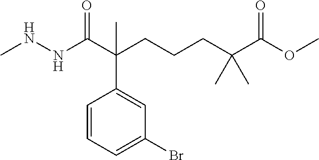 | methyl 6-(3-bromophenyl)- 2,2,6-trimethyl-7-(2- methylhydrazineyl)-7- oxoheptanoate | 399, 401 |
| 67-6 | 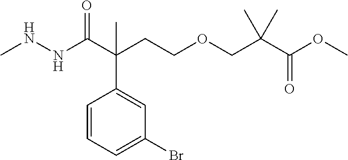 | methyl 3-(3-(3- bromophenyl)-3-methyl-4- (2-methylhydrazineyl)-4- oxobutoxy)-2,2- dimethylpropanoate | 415, 417 |
| 67-7 | 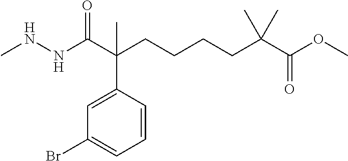 | methyl 7-(3-bromophenyl)- 2,2,7-trimethyl-8-(2- methylhydrazineyl)-8- oxooctanoate | 413, 415 |
| 67-8 | 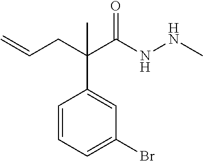 [SMILES: C=CCC(C)(C(=O)NNC)c1cccc(Br)c1] | 2-(3-bromophenyl)-N′,2- dimethylpent-4- enehydrazide | 297, 299 |
| 67-9 | 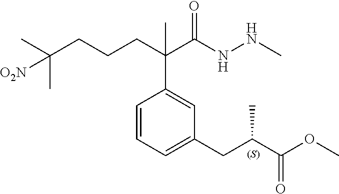 | methyl (2S)-3-(3-(2,6- dimethyl-1-(2- methylhydrazineyl)-6-nitro- 1-oxoheptan-2-yl)phenyl)-2- methylpropanoate | 408 |
| 67-10 | 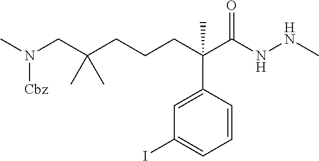 | benzyl (R)-(6-(3-iodophenyl)- 2,2,6-trimethyl-7-(2- methylhydrazineyl)-7- oxoheptyl)(methyl)carbamate | 566 |
| 67-11 | 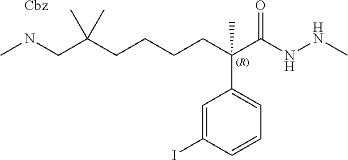 | benzyl (R)-(7-(3-iodophenyl)- 2,2,7-trimethyl-8-(2- methylhydrazineyl)-8- oxooctyl)(methyl)carbamate | 580 |

### Intermediate 67A: Tert-Butyl 2-(2-(3-bromophenyl)-8-methoxy-7,7-dimethyl-8-oxooctanoyl)-1-methylhydrazine-1-carboxylate

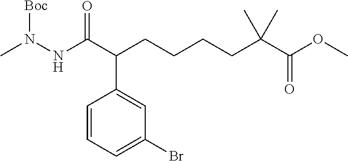

To a stirred solution of 2-(3-bromophenyl)-8-methoxy-7,7-dimethyl-8-oxooctanoic acid (Intermediate 40A-1, 11 g, 0.02906 mol) in acetonitrile (200 mL) was added 1-methyl-1H-imidazole (8.51 g, 0.104 mol), tert-butyl 1-methylhydrazine-1-carboxylate (4.76 g, 0.0326 mol) and N,N,N′,N′-tetramethylchloroformamidinium hexafluorophosphate (TCFH) (9.98 g, 0.356 mol). The mixture was stirred for two hours at room temperature and concentrated. The residue was dissolved in ethyl acetate (200 mL), washed with water, brine, dried over sodium sulfate, and concentrated. The crude product was purified by silica gel column chromatography (petroleum ether/ethyl acetate=1:1) to give the title compound (14 g, 95%) as a light-yellow oil. MS (ESI): 521, 523 m/z [M+Na]⁺, retention time: 2.32 minutes, purity: 96% (214 nm) (LC-MS method 14).

### Intermediate 68: N,2-Dimethylpent-4-en-2-amine hydrochloride

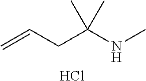

To a stirred solution of tert-butyl methyl(2-methylpent-4-en-2-yl)carbamate (Intermediate 68C, 5.70 g, 26.7 mmol) in 1,4-dioxane (30 mL) was added hydrogen chloride (4M in dioxane, 26.7 mL, 0.107 mol). The mixture was stirred at room temperature overnight and concentrated to afford the crude title compound (2.80 g, 93%) as a light-yellow solid. This crude compound was used for next step without further purification. ¹H NMR (400 MHz, CDCl₃) δ 9.40 (br, 2H), 5.90-5.80 (m, 1H), 5.27-5.14 (m, 2H), 2.60 (t, J=5.2 Hz, 3H), 2.52 (d, J=7.6 Hz, 2H), 1.42 (s, 6H) ppm.

### Intermediate 68A: 2,2-Dimethylpent-4-enoic acid

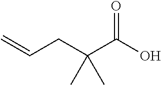

To a stirred and cooled (−78° C.) solution of 2-methylpropanoic acid (5 g, 0.0568 mol) in N,N-dimethylformamide (50 mL) was added lithium diisopropylamide (2 M in tetrahydrofuran, 85.1 mL, 0.170 mol). The mixture was stirred at room temperature for 2 hours, treated with 3-bromoprop-1-ene (20.6 g, 0.170 mol), and stirred at room temperature overnight. The reaction was quenched with 200 mL of water, extracted with ethyl acetate (3×200 mL). The combined organic phases were washed with brine, dried over sodium sulfate and concentrated. The residue was purified by silica gel column chromatography (eluting with petroleum ether: ethyl acetate=8:1) to give the title compound (5.10 g, 70%) as a colorless liquid. ¹H NMR (400 MHz, CDCl₃) δ 5.85-5.74 (m, 1H), 5.12-5.07 (m, 2H), 2.29 (d, J=7.2 Hz, 2H), 1.22 (s, 6H) ppm.

### Intermediate 68B: tert-butyl (2-methylpent-4-en-2-yl)carbamate

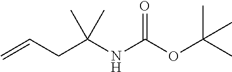

To a stirred solution of 2,2-dimethylpent-4-enoic acid (Intermediate 68A, 5.1 g, 0.0398 mol) in tetrahydrofuran (60 mL) was added sodium azide (9.05 g, 0.139 mol), di-tert-butyl dicarbonate (13 g, 1.50 mol), tetrabutyl ammonium bromide (1.92 g, 5.97 mmol) and zinc bromide (0.296 g, 1.31 mmol). The mixture was stirred at 40° C. overnight, cooled to room temperature, quenched with 10% solution of sodium nitrite (80 mL), and diluted with ethyl acetate (50 mL). The solution was stirred for 20 minutes and extracted with ethyl acetate (3×80 mL). The combined organic layers were washed with saturated ammonium chloride, saturated sodium bicarbonate, brine. dried over sodium sulfate and concentrated. The residue was purified by silica gel column chromatography (eluting with petroleum ether: ethyl acetate=20:1) to give the title compound (6.50 g, 82%) as a colorless liquid. ¹H NMR (400 MHz, CDCl₃) δ 5.86-5.75 (m, 1H), 5.13-5.07 (m, 2H), 4.45 (br, 1H), 2.41 (d, J=7.6 Hz, 2H), 1.45 (s, 9H), 1.28 (s, 6H) ppm.

### Intermediate 68C: Tert-Butyl methyl(2-methylpent-4-en-2-yl)carbamate

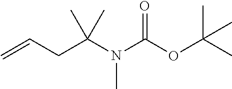

To a stirred and cooled (0° C.) solution of tert-butyl (2-methylpent-4-en-2-yl)carbamate (Intermediate 68B, 6.50 g, 0.0326 mol) in N,N-dimethylformamide (50 mL) was added sodium hydride (3.91 g, 0.0978 mol). The mixture was stirred for 1 hour at 0° C., treated with iodomethane (10.2 mL, 0.163 mol), and stirred at room temperature overnight. The reaction was quenched with 100 mL of water and extracted with ethyl acetate (3×100 mL). The combined organic phases were washed with brine, dried over sodium sulfate, and concentrated. The residue was purified by silica gel column chromatography (eluting with petroleum ether: ethyl acetate=4:1) to give the title compound (5.7 g, 80%) as a yellow liquid. ¹H NMR (400 MHz, CDCl₃) δ 5.81-5.74 (m, 1H), 5.09-5.04 (m, 2H), 2.86 (s, 3H), 2.58 (d, J=7.2 Hz, 2H), 1.48 (s, 9H), 1.35 (s, 6H) ppm.

### Intermediate 69: 1-Iodo-4-methyl-4-nitropentane

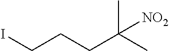

To a stirred solution of 4-methyl-4-nitropentan-1-ol (Intermediate 69B, 21.3 g, 0.145 mol) and sodium iodide (43.4 g, 0.29 mol) in 200 mL of acetonitrile was added trimethylchlorosilane (36.8 mL, 0.29 mol) drop wise. The reaction mixture was stirred overnight and filtered. The filtrate was concentrated. The residue was mixed with diethyl ether (200 mL), washed with saturate sodium bisulfite solution, water, brine, dried over sodium sulphate, and concentrated. The residue was purified by automated silica gel column chromatography (eluting with petroleum ether: ethyl acetate=0˜60%, 300 g silica gel column) to give the title compound (19 g, 51%) as an oil. MS (ESI): 211 m/z [M-NO₂]⁺, retention time: 1.87 minutes, purity: 99% (214 nm) (LC-MS method 25).

### Intermediate 69A: Ethyl 4-methyl-4-nitropentanoate

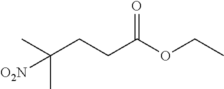

To a stirred solution of 2-nitropropane (25 g, 0.28 mol) in ethanol (140 ml) was added ethyl acrylate (28 g, 0.28 mol) and potassium fluoride (1.63 g, 0.028 mol). The mixture was refluxing for 4 hours and concentrated. The residue was diluted with water (50 mL) and extracted with ethyl acetate (2×100 mL). The combined organic extracts were washed with brine, dried over sodium sulfate and concentrated to afford the crude title compound (38.5 g, crude) as an oil. MS (ESI): 212 m/z [M+Na]⁺, retention time: 1.89 minutes, purity: 95% (214 nm) (LC-MS method 25).

### Intermediate 69B: 4-Methyl-4-nitropentan-1-ol

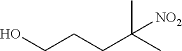

To a stirred and cooled (0° C.) suspension of sodium borohydride (13.9 g, 0.36 mol) in tetrahydrofuran (200 mL) was added a solution of ethyl 4-methyl-4-nitropentanoate (Intermediate 69A, 38.5 g, crude) in 50 mL of ethanol drop wise. The mixture was stirred at room temperature for 4 hours, then refluxed for 6 hours. The mixture was cooled to room temperature and neutralized with 36% hydrochloric acid to pH˜7. The mixture was filtered. The filter cake was washed with tetrahydrofuran (3×50 mL). The combined filtrate was concentrated. The residue was purified by silica gel column chromatography (330 g silica gel column, eluting with 0-10% ethyl acetate in petroleum ether) to give the title compound (23.1 g, 77%) as oil. MS (ESI): 170 m/z [M+Na]⁺, retention time: 1.53 minutes, purity: 99% (214 nm) (LC-MS method 25).

### Intermediate 70: Tert-Butyl 2-(3-((S)-3-methoxy-2-methyl-3-oxopropyl)phenyl)-2,6-dimethyl-6-nitroheptanoate

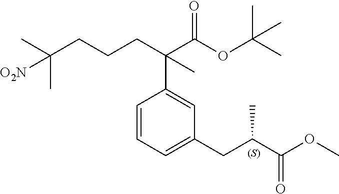

To a suspension of zinc (10.2 g, 156 mmol) in N,N-dimethylformamide (250 mL) was added iodine (660 mg, 2.6 mmol). The mixture was stirred at room temperature for 40 minutes, then treated with methyl (2S)-3-iodo-2-methyl-propanoate (8.89 g, 39 mmol), and stirred for another 40 minutes. To this solution was added 2-dicyclohexylphosphino-2′,6′-dimethoxy-1,1′-biphenyl (1 g, 2.6 mmol) and tris(dibenzylideneacetone)dipalladium (1.19 g, 1.3 mmol), followed by tert-butyl 2-(3-iodophenyl)-2,6-dimethyl-6-nitroheptanoate (Intermediate 19A-37, 12 g, 26 mmol). The reaction mixture was stirred at room temperature for 16 hours and filtered. The filter cake was washed with ethyl acetate (200 mL). The combined filtrate was washed with water (2×100 mL). The combined aqueous wash was back extracted with ethyl acetate (50 mL×3). The combined organic extracts were washed with brine, dried over sodium sulfate, and concentrated. The residue was purified by automated flash chromatography (40 g silica gel column, eluting with 0-30% ethyl acetate in petroleum ether) to give the title compound (7.9 g, 70%) as an oil. MS (ESI): 458 m/z [M+Na]⁺, retention time: 2.13 minutes, purity: 94% (214 nm) (LC-MS method 25).

### Intermediate 71: 6-Acetoxy-7-azido-2-(3-iodophenyl)-2-methylheptanoic acid

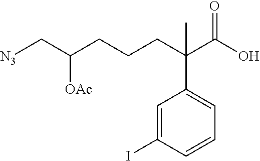 [SMILES: CC(=O)OC(CCCC(C)(C(=O)O)c1cccc(I)c1)CN=[N+]=[N-]]

To the stirred solution of 7-azido-6-hydroxy-2-(3-iodophenyl)-2-methylheptanoic acid (Intermediate 71D, 1 g, 2.6 mmol) in acetic anhydride (5 mL) was added p-toluenesulfonic acid (30 mg, 0.2 mmol). The reaction was stirred for 4 hours, treated with water/tetrahydrofuran (100 mL, 10/1) and stirred for another 1 hour. The mixture was concentrated. The residue was partitioned between water (50 mL) and ethyl acetate (100 mL). The separated organic layer was washed with brine, dried over magnesium sulfate and concentrated. The crude product was purified by automated flash chromatography (eluting with 30% ethyl acetate/petroleum ether) to give the title compound (900 mg, 70%) as a white solid. MS (ESI): 468 m/z [M+Na]⁺, retention time: 1.56 minutes, purity: 95% (254 nm) (LC-MS method 2).

The following intermediate was prepared based on the procedures described for Intermediate 71.

| Inter. | | | MS m/z |
|---|---|---|---|
| No. | Structure | Name | [M + H]⁺ |
| 71-1 | 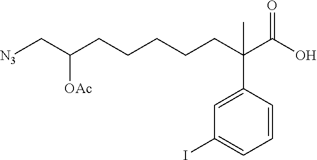 [SMILES: CC(=O)OC(CCCCCC(C)(C(=O)O)c1cccc(I)c1)CN=[N+]=[N-]] | 8-acetoxy-9-azido-2-(3- iodophenyl)-2- methylnonanoic acid | 474 |

### Intermediate 71A: Methyl 6,7-dihydroxy-2-(3-iodophenyl)-2-methylheptanoate

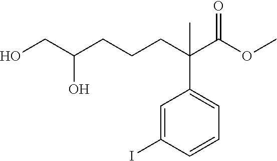 [SMILES: COC(=O)C(C)(CCCC(O)CO)c1cccc(I)c1]

To the stirred solution of methyl 2-(3-iodophenyl)-2-methylhept-6-enoate (Intermediate 19A-39, 10 g, 28 mmol) and N-methylmorpholine N-oxide (4.9 g, 42 mmol) in 100 mL of tetrahydrofuran was added osmium tetroxide (100 mg, 4% in water) at room temperature. The mixture was stirred for 24 hours and concentrated. The residue was partitioned between water (150 mL) and ethyl acetate (150 mL). The separated organic layer, combined with two additional ethyl acetate (2×150 mL) extracts, was washed with brine, dried over magnesium sulfate, and concentrated. The crude product was purified by automated flash chromatography (80 g silica gel column, eluting with 30% ethyl acetate/petroleum ether) to give the title compound (9 g, 85%) as a white solid. MS (ESI): 393 m/z [M+H]⁺, retention time: 1.56 minutes, purity: 95% (214 nm) (LC-MS method 2).

The following intermediate was prepared based on the procedures described for Intermediate 71A.

| Inter. | | | MS m/z |
|---|---|---|---|
| No. | Structure | Name | [M + H]⁺ |
| 71A-1 | 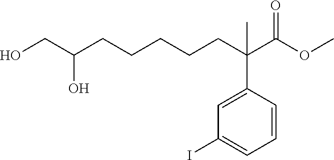 [SMILES: COC(=O)C(C)(CCCCCC(O)CO)c1cccc(I)c1] | methyl 8,9-dihydroxy-2-(3- iodophenyl)-2- methylnonanoate | 421 |

### Intermediate 71B: Ethyl 6-hydroxy-2-(3-iodophenyl)-2-methyl-7-((methylsulfonyl)oxy)-heptanoate

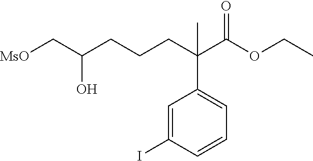 [SMILES: CCOC(=O)C(C)(CCCC(O)COS(C)(=O)=O)c1cccc(I)c1]

To a stirred solution of ethyl 6,7-dihydroxy-2-(3-iodophenyl)-2-methylheptanoate (Intermediate 71A, 3.5 g, 8.62 mmol) and triethylamine (1.74 g, 17.2 mmol) in 200 mL of dichloromethane was added methanesulfonyl chloride (879 mg, 8.62 mmol) dropwise over 5 minutes at room temperature. The mixture was stirred for 16 hours, then quenched with ice-water (100 mL). The separated organic layer was washed with saturated potassium carbonate, brine, dried over sodium sulfate, and concentrated to give the crude title compound (3.9 g, 98%) as a yellow oil. The crude product was used for next step without further purification. MS (ESI): 485 m/z [M+H]⁺, retention time: 1.91 minutes, purity: 78% (254 nm) (LC-MS method 2).

The following intermediate was prepared based on the procedures described for Intermediate 71B.

| Inter. | | | MS m/z |
|---|---|---|---|
| No. | Structure | Name | [M + H]⁺ |
| 71B-1 | 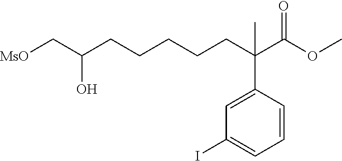 [SMILES: COC(=O)C(C)(CCCCCC(O)COS(C)(=O)=O)c1cccc(I)c1] | methyl 8-hydroxy-2-(3- iodophenyl)-2-methyl-9- ((methylsulfonyl)oxy)nonanoate | 499 |

### Intermediate 71C: Ethyl 7-azido-6-hydroxy-2-(3-iodophenyl)-2-methylheptanoate

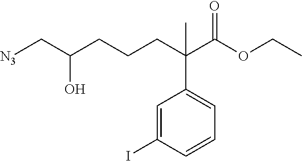 [SMILES: CCOC(=O)C(C)(CCCC(O)CN=[N+]=[N-])c1cccc(I)c1]

To a stirred solution of ethyl 6-hydroxy-2-(3-iodophenyl)-2-methyl-7-((methylsulfonyl)oxy)-heptanoate (Intermediate 71B, 500 mg, 1.05 mmol) in 5 mL of dimethylformamide was added sodium azide (137 mg, 2.10 mmol). The mixture was stirred at 80° C. for 8 hours, quenched with ice-water (20 mL), and diluted with ethyl acetate (50 mL). The separated organic layer was washed with brine, dried over sodium sulfate, and concentrated. The residue was purified by automated flash chromatography (eluting with petroleum ether/ethyl acetate=1/1) to give the title compound (0.4 g, 75%) as a yellow oil. MS (ESI): 432 m/z [M+H]⁺, retention time: 1.94 minutes, purity: 78% (254 nm) (LC-MS method 2).

The following intermediate was prepared based on the procedures described for Intermediate 71C.

| Inter. | | | MS m/z |
|---|---|---|---|
| No. | Structure | Name | [M + H]⁺ |
| 71C-1 | 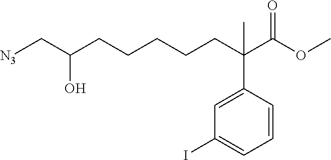 [SMILES: COC(=O)C(C)(CCCCCC(O)CN=[N+]=[N-])c1cccc(I)c1] | methyl 9-azido-8-hydroxy-2- (3-iodophenyl)-2- methylnonanoate | 446 |

### Intermediate 71D: 7-Azido-6-hydroxy-2-(3-iodophenyl)-2-methylheptanoic acid

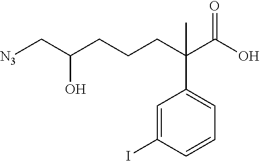 [SMILES: CC(CCCC(O)CN=[N+]=[N-])(C(=O)O)c1cccc(I)c1]

To a stirred solution of ethyl 7-azido-6-hydroxy-2-(3-iodophenyl)-2-methylheptanoate (Intermediate 71C, 1 g, 2.35 mmol) in tetrahydrofuran (10 mL) was added lithium hydroxide (1.88 g, 47.1 mmol). The reaction was stirred for 18 hours, then acidified to pH˜4 with 1 N hydrochloric acid. The mixture was extracted with ethyl acetate (2×50 mL). The combined organic phases were dried over magnesium sulfate and concentrated to provide the title compound (0.8 g, 90%) as brown oil. MS (ESI): 404 m/z [M+H]⁺, retention time: 1.78 minutes, purity: 96% (254 nm) (LC-MS method 2).

The following intermediate was prepared based on the procedures described for Intermediate 71D.

| Inter. | | | MS m/z |
|---|---|---|---|
| No. | Structure | Name | [M + H]⁺ |
| 71D-1 | 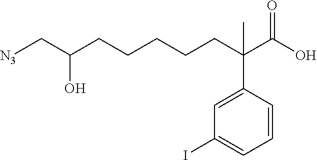 [SMILES: CC(CCCCCC(O)CN=[N+]=[N-])(C(=O)O)c1cccc(I)c1] | 9-azido-8-hydroxy-2-(3- iodophenyl)-2- methylnonanoic acid | 432 |

### Intermediate 72: Methyl 2-(3-iodophenyl)-2-methyl-7-(2-oxooxazolidin-5-yl)heptanoate

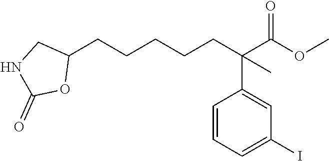 [SMILES: COC(=O)C(C)(CCCCCC1CNC(=O)O1)c1cccc(I)c1]

To a stirred solution of methyl 9-amino-8-hydroxy-2-(3-iodophenyl)-2-methylnonanoate (Intermediate 72C, 4.64 g, 11.1 mmol) in dichloromethane (124 mL) was added 1,1′-carbonyldiimidazole (CDI) (1.88 g, 11.6 mmol) and imidazole (377 mg, 5.55 mmol). The mixture was stirred at room temperature overnight and diluted with ethyl acetate (500 mL). The solution was washed with water, brine, dried over sodium sulfate, and concentrated. The residue was purified by silica gel column chromatography (80 g silica gel column, eluting with 0-10% methanol in dichloromethane) to give the title compound (4.60 g, 93%) as a colorless oil. MS (ESI): 446 m/z [M+H]⁺, retention time: 2.11 minutes, purity: 84% (254 nm) (LC-MS method 4).

### Intermediate 72A: Methyl 9-bromo-8-hydroxy-2-(3-iodophenyl)-2-methylnonanoate

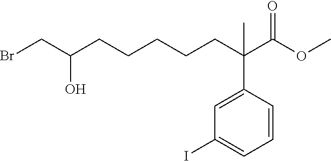 [SMILES: COC(=O)C(C)(CCCCCC(O)CBr)c1cccc(I)c1]

To a stirred and cooled (0° C.) solution of methyl 2-(3-iodophenyl)-2-methylnon-8-enoate (Intermediate 19A-40, 20.38 g, 52.8 mol) in dimethyl sulfoxide (57 mL) was added N-bromo succinimide (10.34 g, 58.1 mmol) and water (1.9 mL, 106 mmol). The reaction was stirred at room temperature for 2 hours and diluted with ethyl acetate (500 mL). The solution was washed with water, brine, dried over sodium sulfate, and concentrated. The residue was purified by silica gel column chromatography (330 g silica gel column, eluting with 0-30% ethyl acetate in petroleum ether) to give the title compound (15.74 g, 62%) as a colorless oil.

### Intermediate 72B: Methyl 9-azido-8-hydroxy-2-(3-iodophenyl)-2-methylnonanoate

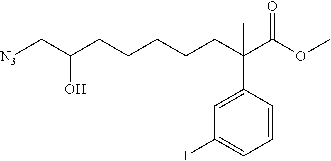 [SMILES: COC(=O)C(C)(CCCCCC(O)CN=[N+]=[N-])c1cccc(I)c1]

To a stirred solution of methyl 9-bromo-8-hydroxy-2-(3-iodophenyl)-2-methylnonanoate (Intermediate 72A, 15.74 g, 32.7 mmol) in N,N-dimethylformamide (97 mL) was added sodium azide (4.25 g, 65.3 mmol). The mixture was stirred at 90° C. overnight, cooled to room temperature, and diluted with ethyl acetate (200 mL). The solution was washed with water, brine, dried over sodium sulfate, and concentrated. The residue was purified by silica gel column chromatography (80 g silica gel column, eluting with 0-30% ethyl acetate in petroleum ether) to give the title compound (11.3 g, 78%) as a colorless oil. MS (ESI): 468 m/z [M+Na]⁺, retention time: 2.16 minutes, purity: 83% (254 nm) (LC-MS method 7).

### Intermediate 72C: Methyl 9-amino-8-hydroxy-2-(3-iodophenyl)-2-methylnonanoate

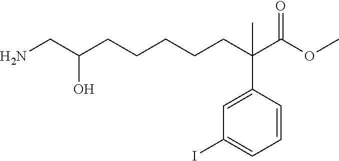 [SMILES: COC(=O)C(C)(CCCCCC(O)CN)c1cccc(I)c1]

To a stirred solution of methyl 9-azido-8-hydroxy-2-(3-iodophenyl)-2-methylnonanoate (Intermediate 72B, 6.19 g, 13.9 mmol) in tetrahydrofuran-water (3:1 (V/V), 62.7 mL) was added triphenylphosphine (4.01 g, 15.3 mmol). The mixture was stirred at 50° C. overnight and diluted with ethyl acetate (500 mL). The solution was washed with water, brine, dried over sodium sulfate, and concentrated. The residue was purified by silica gel column chromatography (80 g silica gel column, eluting with 0-10% methanol in dichloromethane) to give the title compound (4.64 g, 80%) as a colorless oil. MS (ESI): 420 m/z [M+Na]⁺, retention time: 1.47 minutes, purity: 83% (254 nm) (LC-MS method 4).

### Intermediate 73: Methyl 2-(3-iodophenyl)-2-methyl-7-(3-methyl-2-oxooxazolidin-5-yl)heptanoate

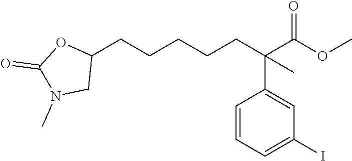 [SMILES: COC(=O)C(C)(CCCCCC1CN(C)C(=O)O1)c1cccc(I)c1]

To a stirred and cooled (0° C.) solution of methyl 2-(3-iodophenyl)-2-methyl-7-(2-oxooxazolidin-5-yl)heptanoate (Intermediate 72, 4.60 g, 10.3 mmol) in N,N-dimethylformamide (93 mL) was added sodium hydride (828 mg, 20.6 mmol). The mixture was stirred at this temperature for 1 hour, treated with iodomethane (1.92 mL, 30.9 mmol), and continually stirred at room temperature overnight. The reaction mixture was diluted with ethyl acetate (600 mL), washed with water, brine, dried over sodium sulfate, and concentrated. The crude product was purified by silica gel column chromatography (80 g silica gel column, eluting with 0-100% ethyl acetate in petroleum ether) to give the title compound (4.39 g, 93%) as a colorless oil. MS (ESI): 460 m/z [M+H]⁺, retention time: 2.20 minutes, purity: 84% (254 nm) (LC-MS method 4).

### Intermediate 74: 4-((4-Bromo-6-fluoro-1H-indol-5-yl)oxy)picolinonitrile

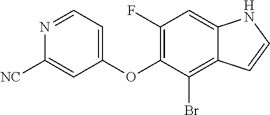 [SMILES: N#Cc1cc(Oc2c(F)cc3[nH]ccc3c2Br)ccn1]

To a stirred solution of 4-(2-bromo-6-fluoro-3-methyl-4-nitro-phenoxy)pyridine-2-carbonitrile (Intermediate 74A, 157.17 g, 446 mmol) in N,N-dimethylformamide (1500 mL) was added N,N-dimethylformamide dimethyl acetal (299 mL, 2232 mmol). The mixture was stirred at 100° C. for 6 hours and concentrated to afford (E)-4-(2-bromo-3-(2-(dimethylamino)vinyl)-6-fluoro-4-nitrophenoxy)picolinonitrile as a brown residue.

To a stirred solution of the above brown residue in methanol (600 mL) and tetrahydrofuran (600 mL) was added stannous dichloride dihydrate (175.75 g, 772 mmol). The mixture was stirred at room temperature for 3 hours, then adjusted the pH to ˜7 using 1N sodium hydroxide and filtered. The filtrate was extracted with ethyl acetate (2×700 mL). The combined organic phase was washed with brine, dried over sodium sulfate, filtered, and concentrated to provide crude 4-((4-Bromo-6-fluoro-1-hydroxy-1H-indol-5-yl)oxy)picolinonitrile. LC-MS: MS (ESI): 348, 350 m/z [M+H]⁺, retention time: 1.78 minutes, purity: >99% (254 nm) (LC-MS method 25).

To a stirred solution of 2-bromo-1-phenyl-ethanone (89.86 g, 451 mmol) in methanol (1500 mL) was added the above crude 4-(4-bromo-6-fluoro-1-hydroxy-indol-5-yl)oxypyridine-2-carbonitrile (157.17 g, 451 mmol) and triethylamine (145 mL, 1038 mmol). The mixture was stirred for 2 hours at room temperature, then concentrated. The residue was purified by column chromatography (80% ethyl acetate in petroleum ether, silica gel column) to afford the title compound (78.00 g, 170 mmol, 38%) as a yellow solid. LC-MS: MS (ESI): 332, 334 m/z [M+H]⁺, retention time: 1.81 minutes, purity: 72% (254 nm) (LC-MS method 25).

### Intermediate 74A: 4-(2-Bromo-6-fluoro-3-methyl-4-nitrophenoxy)picolinonitrile

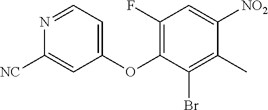 [SMILES: Cc1c([N+](=O)[O-])cc(F)c(Oc2ccnc(C#N)c2)c1Br]

To a stirred solution of 4-hydroxypyridine-2-carbonitrile (108.11 g, 900 mmol) in N,N-dimethylformamide (1080 mL) was added 3-bromo-1,2-difluoro-4-methyl-5-nitro-benzene (226.83 g, 900 mmol) and potassium carbonate (311.00 g, 2250 mmol). The mixture was stirred at room temperature for 24 hours, diluted with 2.5 L of ethyl acetate. The solution was washed with water, brine, dried over sodium sulfate, and concentrated. The residue was purified by column chromatography (20% ethyl acetate in petroleum ether, silica gel column) to afford the title compound (157.17 g, 446 mmol, 50%) as a yellow solid. LC-MS: MS (ESI): 352, 354 m/z [M+H]⁺, retention time: 1.82 minutes, purity: >99% (254 nm) (LC-MS method 25).

### Intermediate 75: Ethyl (E)-3-(6-fluoro-5-((2-(imino(methylthio)methyl)pyridin-4-yl)oxy)-1H-indol-4-yl)acrylate

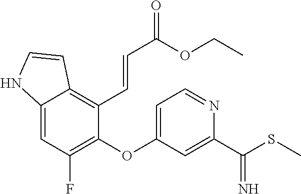 [SMILES: CCOC(=O)/C=C/c1c(Oc2ccnc(C(=N)SC)c2)c(F)cc2[nH]ccc12]

To a stirred suspension of sodium bicarbonate (0.057 g, 0.683 mmol) in acetone (5 mL) were added (E)-4-((4-(3-ethoxy-3-oxoprop-1-en-1-yl)-6-fluoro-1H-indol-5-yl)oxy)pyridine-2-carbimidothioic acid (Intermediate 65A-1, 0.050 g, 0.137 mmol) and methyl iodide (0.043 mL, 0.683 mmol). The reaction mixture was stirred at room temperature for 4 hours and concentrated. The residue was taken up in ethyl acetate (10 mL), washed with water, brine, dried over sodium sulfate, and concentrated to give the title compound (48 mg, 0.114 mmol, 83%) as a yellow solid. LC-MS: MS (ESI): 400 m/z [M+H]⁺, retention time: 1.65 minutes, purity: 90% (214 nm) (LC-MS method 25).

The following intermediate was prepared based on the procedures described for Intermediate 75.

| Inter. | | | MS m/z |
|---|---|---|---|
| No. | Structure | Name | [M + H]⁺ |
| 75-1 | 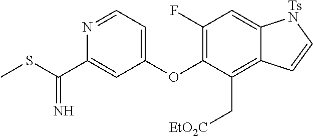 [SMILES: CCOC(=O)Cc1c(Oc2ccnc(C(=N)SC)c2)c(F)cc2c1ccn2S(=O)(=O)c1ccc(C)cc1] | ethyl 2-(6-fluoro-5-((2- (imino(methylthio)methyl) pyridin-4-yl)oxy)-1-tosyl-1H- indol-4-yl)acetate | 542 |

### Intermediate 76: (R)-7-Hydroxy-2-(3-iodophenyl)-2,6,6-trimethylheptanoic acid

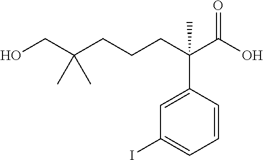 [SMILES: CC(C)(CO)CCC[C@@](C)(C(=O)O)c1cccc(I)c1]

A chiral salt of enantiomer 1 of (S)-1-(naphthalen-1-yl)ethan-1-aminium 7-hydroxy-2-(3-iodophenyl)-2,6,6-trimethylheptanoate (Intermediate 135B, 233 g, 415 mmol) was suspended in ethyl acetate (1000 mL). The mixture was washed with 1N HCl (5×500 mL), brine, dried over anhydrous sodium sulfate, and concentrated to give the title compound (160.00 g, 99%) as light yellow oil. LC-MS: MS (ESI): 413 m/z [M+Na]⁺, retention time: 1.98 minutes, purity: 89% (214 nm) (LC-MS Method 003).

### Intermediate 76A: ˜90% ee of (S)-1-(Naphthalen-1-yl)ethan-1-aminium (R)-7-hydroxy-2-(3-iodophenyl)-2,6,6-trimethylheptanoate

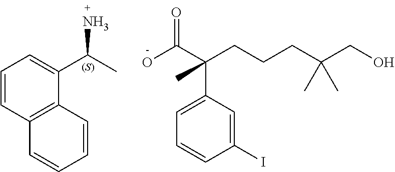

To a stirred solution of (S)-(−)-1-(1-naphthyl)ethylamine (127 mL, 705 mmol) in ethyl acetate (7.5 L) was added 7-hydroxy-2-(3-iodophenyl)-2,6,6-trimethyl-heptanoic acid (Intermediate 17B, 500.00 g, 1281 mmol). The reaction was stirred at room temperature overnight. The solid was collected by filtration and the filtrate cake was washed with ethyl acetate (800 mL). The wet solid was then suspended into EtOAc (1.7 L) and stirred at RT for 20 minutes. The mixture was filtered and washed with ethyl acetate (800 mL). The solid was collected and dried by air to give Enantiomer 1 of (S)-1-(naphthalen-1-yl)ethan-1-aminium 7-hydroxy-2-(3-iodophenyl)-2,6,6-trimethyl-heptanoate (288 g, 40%). Chiral purity: 95% (90% ee). (Chiral conditions: column: AD-H; Mobile phase: n-hexane (0.1% diethylamine): ethanol (0.1% diethylamine)=95:5; Column temperature: 40° C.; Flow rate: 1.0 mL/minute; Wavelength: 220 nm; Instrument: Shimadzu; Solid salt were de-salted with 1N hydrochloric acid in acetonitrile before injection)

### Intermediate 76B: ˜98% ee of (S)-1-(Naphthalen-1-yl)ethan-1-aminium (R)-7-hydroxy-2-(3-iodophenyl)-2,6,6-trimethylheptanoate

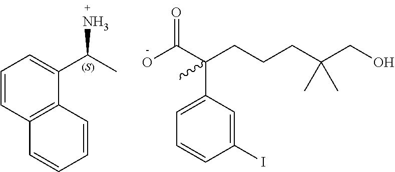

A solution of ˜90% ee of Enantiomer 1 of (S)-1-(naphthalen-1-yl)ethan-1-aminium 7-hydroxy-2-(3-iodophenyl)-2,6,6-trimethylheptanoate (Intermediate 135A, 186 g, 315 mmol) in acetonitrile (7.65 L) was refluxed for 5 hours and then stirred at room temperature overnight. The mixture was filtered and rinsed with acetonitrile (2×450 mL). The solid was collected and dried in vacuo to give the title compound (153 g, 86%). Chiral purity: 99% (98% ee, chiral method is the same as in Intermediate 135A). ¹H NMR (400 MHz, CDCl₃) δ 7.84 (t, J=6.7 Hz, 2H), 7.77 (d, J=8.2 Hz, 1H), 7.67 (t, J=1.6 Hz, 1H), 7.56 (d, J=7.1 Hz, 1H), 7.52-7.37 (m, 4H), 7.19 (d, J=8.5 Hz, 1H), 6.92 (t, J=7.9 Hz, 1H), 4.85 (q, J=6.6 Hz, 1H), 3.19 (dd, J=42.8, 10.4 Hz, 2H), 1.77-1.66 (m, 1H), 1.61-1.57 (m, 1H), 1.54 (d, J=6.7 Hz, 3H), 1.38 (s, 3H), 1.28-0.98 (m, 4H), 0.79 (s, 3H), 0.74 (s, 3H) ppm.

### Intermediate 77: Ethyl 2-(5-((2-cyanopyridin-4-yl)oxy)-6-fluoro-1-tosyl-1H-indol-4-yl)acetate

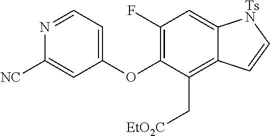 [SMILES: CCOC(=O)Cc1c(Oc2ccnc(C#N)c2)c(F)cc2c1ccn2S(=O)(=O)c1ccc(C)cc1]

To a stirred solution of 4-((4-bromo-6-fluoro-1-tosyl-1H-indol-5-yl)oxy)picolinonitrile (0.50 g, 1.03 mmol) dimethylformamide (12 mL) was added ethyl 2-tributylstannylacetate (1.55 g, 4.01 mmol) and dichlorobis(tri-o-tolylphosphine)palladium(II) (80 mg, 0.103 mmol) in a 20 mL microwave reaction vial. The mixture was heated to 100° C. for 40 minutes in a microwave reactor. Eight same sized reactions were repeated. The combined reaction mixture was diluted with water (500 mL) and extracted with ethyl acetate (2×200 mL). The combined organic extracts were washed with brine, dried over sodium sulfate, filtered, and concentrated. The residue was purified by automated flash chromatograph (eluting with 0 to 30% ethyl acetate in petroleum ether) to give the title compound (1.80 g, 3.54 mmol, 43%) as a white solid. LC-MS: MS (ESI): 494 m/z [M+Na]⁺, retention time: 1.98 minutes, purity: 97% (254 nm) (LC-MS Method 025).

### Preparation of Exemplified Compounds

### Example 1. Example 1. 3-[3-(23,29-difluoro-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

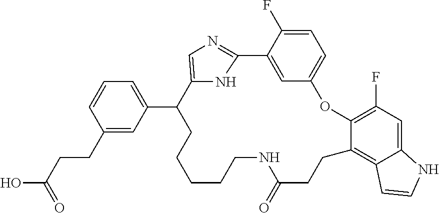 [SMILES: O=C(O)CCc1cccc(C2CCCCCNC(=O)CCc3c(c(F)cc4[nH]ccc34)Oc3ccc(F)c(c3)-c3ncc2[nH]3)c1]

### Methyl (E)-3-(5-(3-(5-(5-cyano-1-(3-iodophenyl)pentyl)-1H-imidazol-2-yl)-4-fluorophenoxy)-6-fluoro-1H-indol-4-yl)acrylate

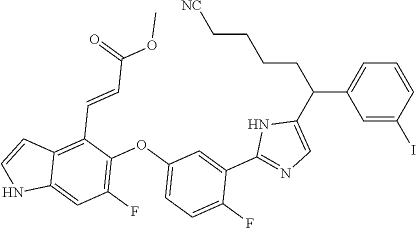 [SMILES: COC(=O)/C=C/c1c(Oc2ccc(F)c(-c3ncc(C(CCCCC#N)c4cccc(I)c4)[nH]3)c2)c(F)cc2[nH]ccc12]

Step A: To a stirred solution of methyl (E)-3-(5-(3-carbamimidoyl-4-fluorophenoxy)-6-fluoro-1H-indol-4-yl)acrylate (Intermediate 5, 1.4 g, 3.77 mmol) and 8-bromo-6-(3-iodophenyl)-7-oxooctanenitrile (Intermediate 1-1, 1.05 g, 2.5 mmol) in N,N-dimethylformamide (30 mL) was added sodium bicarbonate (475 mg, 5.66 mmol). The mixture was stirred at 80° C. for 3 hours. Another portion of methyl (E)-3-(5-(3-carbamimidoyl-4-fluorophenoxy)-6-fluoro-1H-indol-4-yl)acrylate (Intermediate 5, 0.53 g, 1.26 mmol) was added and stirred at 80° C. for an additional 1 hour. The reaction mixture was quenched with water (100 mL) and extracted with ethyl acetate (3×40 mL). The combined organic extracts were washed with brine (50 mL), dried over sodium sulfate, and concentrated. The residue was purified by automated flash chromatography (40 g silica gel column, eluting with 0-100% ethyl acetate in petroleum ether) to give the title compound (1.3 g, 45%) as a yellow solid. MS (ESI): 693 m/z [M+H]⁺, retention time: 1.71 minutes, purity: 90% (254 nm) (LC-MS method 3).

### (E)-3-(5-(3-(5-(5-Cyano-1-(3-iodophenyl)pentyl)-1H-imidazol-2-yl)-4-fluorophenoxy)-6-fluoro-1H-indol-4-yl)acrylic acid

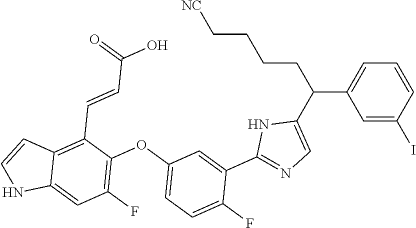 [SMILES: N#CCCCCC(c1cccc(I)c1)c1cnc(-c2cc(Oc3c(F)cc4[nH]ccc4c3/C=C/C(=O)O)ccc2F)[nH]1]

Step B: To a stirred solution of Step A product (1.3 g, 1.88 mmol) in methanol (10 mL) and tetrahydrofuran (30 mL) was added lithium hydroxide monohydrate (1M in water, 10 mL). The mixture was stirred at room temperature overnight, acidified with 1 N hydrochloric acid to pH˜4, and concentrated to remove tetrahydrofuran and methanol. The aqueous residue was diluted with water (50 mL), extracted with ethyl acetate (3×50 mL). The combined organic layers were washed with brine, dried over sodium sulfate, and concentrated to give the title compound (1.2 g, 94%) as a solid. MS (ESI): 679 m/z [M+H]⁺, retention time: 1.55 minutes, purity: 89% (214 nm) (LC-MS method 3).

### (E)-3-(5-(3-(5-(5-Cyano-1-(3-((E)-3-methoxy-3-oxoprop-1-en-1-yl)phenyl)pentyl)-1H-imidazol-2-yl)-4-fluorophenoxy)-6-fluoro-1H-indol-4-yl)acrylic acid

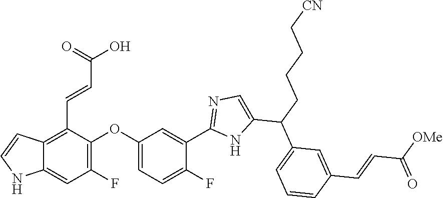 [SMILES: COC(=O)/C=C/c1cccc(C(CCCCC#N)c2cnc(-c3cc(Oc4c(F)cc5[nH]ccc5c4/C=C/C(=O)O)ccc3F)[nH]2)c1]

Step C: In a glove box, to a solution of Step B product (600 mg, 0.89 mmol) in N,N-dimethylformamide (20 mL) was added methyl acrylate (228 mg, 2.66 mmol), tri(o-tolyl)-phosphine (81 mg, 0.266 mmol), palladium (II) acetate (60 mg, 0.266 mmol) and triethylamine (370 uL, 2.66 mmol). The reaction was stirred at 110° C. overnight. Another portion of methyl acrylate (228 mg, 2.66 mmol) was added and stirred at 110° C. for an additional 4 hours. The mixture was cooled to room temperature and diluted with ethyl acetate (100 mL). The solution was washed with water, brine, dried over sodium sulfate, and concentrated. The residue was purified by flash chromatography (20 g silica gel column, eluting with 0-10% methanol in dichloromethane) to give the title compound (305 mg, 54%) as a yellow solid. MS (ESI): 637 m/z [M+H]⁺, retention time: 1.49 minutes, purity: 95% (214 nm) (LC-MS method 5).

### Mixture of (E)-3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)acrylic acid and 3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)propanoic acid

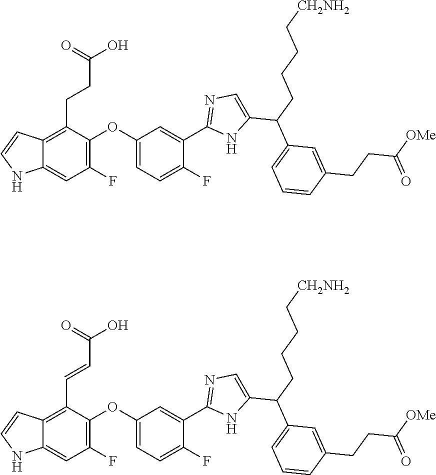

Step D: To a stirred solution of Step C product (305 mg, 0.48 mmol) in tetrahydrofuran (45 mL) was added concentrated ammonia (15 mL) and Ranney Nickel (1 g). The mixture was stirred under hydrogen atmosphere at 35° C. overnight, then filtered through a pad of Celite. The filter cake was washed with tetrahydrofuran (2×15 mL) and water (2×15 mL). The filtrate was concentrated to give the crude title compound (270 mg) as a solid, which was used for next step without further purification. MS (ESI): 643 and 645 m/z [M+H]⁺, retention time: 1.30 minutes, purity: 76% (214 nm) (LC-MS method 3).

### Mixture of methyl 3-[3-(23,29-difluoro-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate and methyl 3-[3-[(14E)-23,29-difluoro-13-oxo-25-oxa-3,12,20,31-tetrazapentacyclo[24.3.1.12,5.016,24.017,21]hentriaconta-1(30),2,4,14,16,18,21,23,26,28-decaen-6-yl]phenyl]propanoate

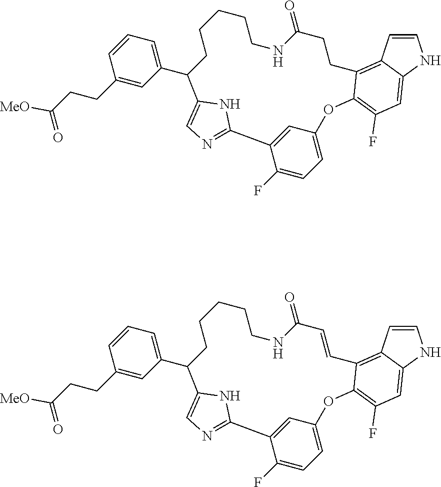 [SMILES: COC(=O)CCc1cccc(C2CCCCCNC(=O)CCc3c(c(F)cc4[nH]ccc34)Oc3ccc(F)c(c3)-c3ncc2[nH]3)c1 ; COC(=O)CCc1cccc(C2CCCCCNC(=O)/C=C/c3c(c(F)cc4[nH]ccc34)Oc3ccc(F)c(c3)-c3ncc2[nH]3)c1]

Step E: To a stirred solution of Step D product (440 mg, 683 μmol) in tetrahydrofuran (120 mL) and N,N-dimethylformamide (60 mL) was added 1-ethyl-3-(3-dimethylaminopropyl)-carbodiimide hydrochloride (261 mg, 1366 μmol) and hydroxy-benzotriazole (184 mg, 1366 μmol). The mixture was stirred at room temperature overnight and concentrated. The residue was diluted with water (30 mL) and extracted with ethyl acetate (5×15 mL). The combined organic extracts were washed with 0.5 N hydrochloric acid, saturated sodium bicarbonate, brine, dried over sodium sulfate, and concentrated to afford the crude title compound (210 mg, 49%) as a yellow solid. MS (ESI): 627 m/z [M+H]⁺, retention time: 1.58 minutes, purity: 37% (214 nm); MS (ESI): 625 m/z [M+H]⁺, retention time: 1.65 minutes, purity: 43% (214 nm). (LC-MS method 3).

### Methyl 3-[3-(23,29-difluoro-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate

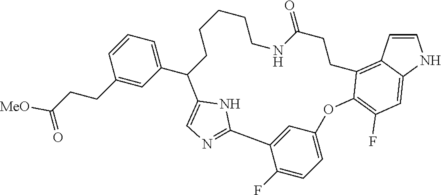 [SMILES: COC(=O)CCc1cccc(C2CCCCCNC(=O)CCc3c(c(F)cc4[nH]ccc34)Oc3ccc(F)c(c3)-c3ncc2[nH]3)c1]

Step F: To a stirred solution of Step E product (270 mg) in tetrahydrofuran (50 mL) was added 10% Pd on carbon (50% wet, 100 mg). The mixture was stirred under hydrogen atmosphere at 35° C. for 2 days, then filtered through a pad of Celite. The filter cake was rinsed with tetrahydrofuran (15 mL). The combined filtrate was concentrated. The residue was purified by automated flash chromatography (20 g silica gel column, eluting with 0-100% ethyl acetate in petroleum ether) to give the title compound (90 mg, 43%) as a solid. MS (ESI): 627 m/z [M+H]⁺, retention time: 1.57 minutes, purity: 95% (254 nm); (LC-MS method 3).

### Compound 1. 3-[3-(23,29-Difluoro-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoic acid

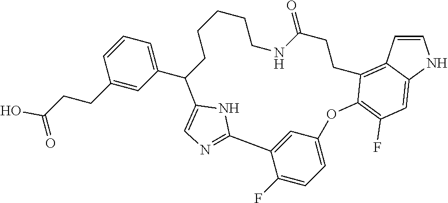 [SMILES: O=C(O)CCc1cccc(C2CCCCCNC(=O)CCc3c(c(F)cc4[nH]ccc34)Oc3ccc(F)c(c3)-c3ncc2[nH]3)c1]

Step G: To a stirred solution of Step F product (50 mg, 79.9 umol) in methanol (3 mL) and tetrahydrofuran (9 mL) was added lithium hydroxide monohydrate (1 M in water, 3 mL). The mixture was stirred at room temperature for 2 hours, then acidified with 1N hydrochloric acid to pH˜4 and diluted with ethyl acetate (50 mL). The solution was washed with brine, dried over sodium sulfate, and concentrated to give the title compound (44.6 mg, 91%) as a white solid. MS (ESI): 613 m/z [M+H]⁺, retention time: 1.66 minutes, purity: 95% (254 nm) (LC-MS method 3). ¹H NMR (400 MHz, CD₃OD) δ 7.88 (d, J=2.8 Hz, 1H), 7.30 (d, J=3.1 Hz, 1H), 7.26 (s, 1H), 7.21-7.12 (m, 4H), 7.08 (s, 1H), 6.97 (s, 1H), 6.83 (d, J=9.0 Hz, 1H), 6.60 (d, J=3.1 Hz, 1H), 4.04-4.00 (m, 1H), 3.19-3.10 (m, 2H), 3.08-2.98 (m, 3H), 2.91 (t, J=7.6 Hz, 2H), 2.62-2.55 (m, 3H), 2.32-2.28 (m, 1H), 2.00-1.94 (m, 1H), 1.54-1.21 (m, 6H) ppm.

### Example 2. Compound 2. 23,29-Difluoro-6-phenyl-25-oxa-3,12,20,31-tetrazapentacyclo[24.3.1.12,5.016,24.017,21]hentriaconta-1(30),2,4,16,18,21,23,26,28-nonaen-13-one

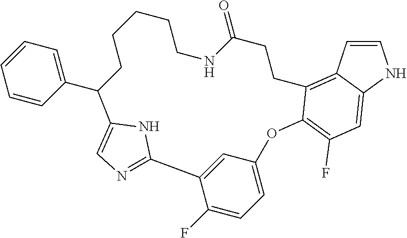 [SMILES: O=C1CCc2c(c(F)cc3[nH]ccc23)Oc2ccc(F)c(c2)-c2ncc([nH]2)C(c2ccccc2)CCCCCN1]

Mixture of 3-(5-(3-(5-(6-amino-1-phenylhexyl)-1H-imidazol-2-yl)-4-fluorophenoxy)-6-fluoro-1H-indol-4-yl)propanoic acid and (E)-3-(5-(3-(5-(6-amino-1-phenylhexyl)-1H-imidazol-2-yl)-4-fluorophenoxy)-6-fluoro-1H-indol-4-yl)acrylic acid

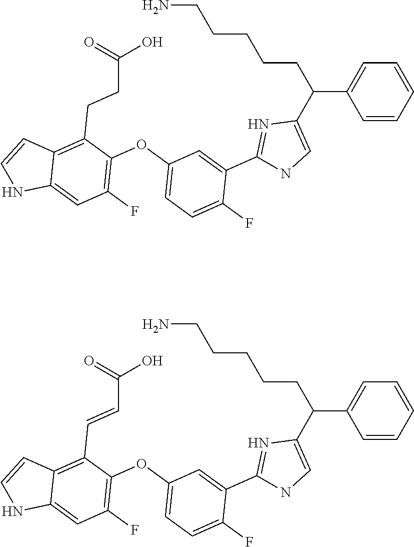 [SMILES: NCCCCCC(c1ccccc1)c1c[nH]c(-c2cc(Oc3c(F)cc4[nH]ccc4c3CCC(=O)O)ccc2F)n1 ; NCCCCCC(c1ccccc1)c1c[nH]c(-c2cc(Oc3c(F)cc4[nH]ccc4c3/C=C/C(=O)O)ccc2F)n1]

Step A: To a stirred solution of (E)-3-(5-(3-(5-(5-cyano-1-(3-iodophenyl)pentyl)-1H-imidazol-2-yl)-4-fluorophenoxy)-6-fluoro-1H-indol-4-yl)acrylic acid (Step B product of Example 1, 200 mg, 0.29 mmol) in tetrahydrofuran (18 mL) and concentrated ammonia (6 mL) was added Raney Ni (500 mg). The mixture was stirred under hydrogen atmosphere at room temperature for 3 days, then filtered through a pad of Celite. The filter cake was rinsed with tetrahydrofuran (30 mL) and water (15 mL). The combined filtrate was concentrated to give the crude title compounds (130 mg) as a solid, which was used for next step without further purification. MS (ESI): 557 and 559 m/z [M+H]⁺, retention time: 1.30 minutes, purity: 98% (254 nm) (LC-MS method 3).

### Compound 2: 23,29-Difluoro-6-phenyl-25-oxa-3,12,20,31-tetrazapentacyclo-[24.3.1.12,5.016,24.017,21]hentriaconta-1(30),2,4,16,18,21,23,26,28-nonaen-13-one

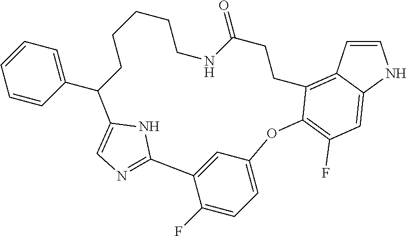 [SMILES: O=C1CCc2c(c(F)cc3[nH]ccc23)Oc2ccc(F)c(c2)-c2ncc([nH]2)C(c2ccccc2)CCCCCN1]

Step B: Exchanging mixture of (E)-3-(5-(3-(5-(6-amino-1-(3-(3-methoxy-3-oxopropyl)phenyl)-hexyl)-1H-imidazol-2-yl)-4-fluorophenoxy)-6-fluoro-1H-indol-4-yl)acrylic acid and 3-(5-(3-(5-(6-amino-i-(3-(3-methoxy-3-oxopropyl)phenyl)hexyl)-1H-imidazol-2-yl)-4-fluorophenoxy)-6-fluoro-1H-indol-4-yl)propanoic acid (Step D product of Example 1) with mixture of 3-(5-(3-(5-(6-amino-i-phenylhexyl)-1H-imidazol-2-yl)-4-fluorophenoxy)-6-fluoro-1H-indol-4-yl)propanoic acid and (E)-3-(5-(3-(5-(6-amino-i-phenylhexyl)-1H-imidazol-2-yl)-4-fluorophenoxy)-6-fluoro-1H-indol-4-yl)acrylic acid (Step A product, 130 mg, 0.233 mmol), the reaction conditions described in Step E of Example 1 were used to prepare the title compound (6.7 mg, purified by prep-HPLC). MS (ESI): 541 m/z [M+H]⁺, retention time: 1.54 minutes, purity: 97% (214 nm) (LC-MS method 3). ¹H NMR (400 MHz, CD₃OD) δ 7.91 (s, 1H), 7.37 (d, J=7.7 Hz, 2H), 7.32-7.26 (m, 3H), 7.23-7.09 (m, 3H), 6.96 (s, 1H), 6.78 (d, J=8.9 Hz, 1H), 6.59 (d, J=3.2 Hz, 1H), 4.08-4.00 (m, 1H), 3.18-2.97 (m, 4H), 2.58-2.54 (m, 2H), 2.32 (s, 1H), 1.98 (s, 1H), 1.42-1.29 (m, 6H) ppm.

### Example 3. Compound 3: 3-[3-(22,28-Difluoro-12-oxo-24-oxa-3,11,19,30-tetrazapentacyclo-[23.3.1.12,5.015,23.016,20]triaconta-1(29),2,4,15,17,20,22,25,27-nonaen-6-yl)phenyl]-propanoic acid

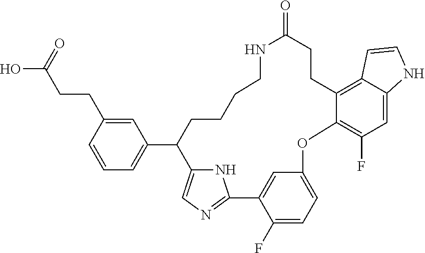 [SMILES: O=C(O)CCc1cccc(C2CCCCNC(=O)CCc3c(c(F)cc4[nH]ccc34)Oc3ccc(F)c(c3)-c3ncc2[nH]3)c1]

Exchanging 8-bromo-6-(3-iodophenyl)-7-oxooctanenitrile (Intermediate 1-1) with 7-bromo-5-(3-iodophenyl)-6-oxoheptanenitrile (intermediate 1, 262 mg, 0.65 mmol), the reaction procedure sequence (Steps A to G) described for Example 1 was used to prepare the title compound (27 mg) as a white solid. MS (ESI): 599 m/z [M+H]⁺, retention time: 1.65 minutes, purity: >99% (254 nm) (LC-MS method 6). ¹H NMR (400 MHz, CD₃OD): δ 7.67-7.65 (m, 1H), 7.27-7.10 (m, 8H), 6.70 (s, 1H), 6.59-6.57 (d, J=4 Hz, 1H), 3.97-3.94 (m, 1H), 3.22-3.19 (m, 2H), 3.15-3.07 (m, 2H), 2.91-2.87 (m, 2H), 2.59-2.55 (m, 2H), 2.45-2.42 (m, 2H), 2.13-2.10 (m, 1H), 1.79-1.76 (m, 1H), 1.58-1.52 (m, 2H), 1.44-1.36 (m, 2H) ppm.

### Example 4. Compound 4. 3-[3-(24,30-Difluoro-14-oxo-26-oxa-3,13,21,32-tetrazapentacyclo-[25.3.1.12,5.017,25.018,22]dotriaconta-1(31),2,4,17,19,22,24,27,29-nonaen-6-yl)phenyl]propanoic acid

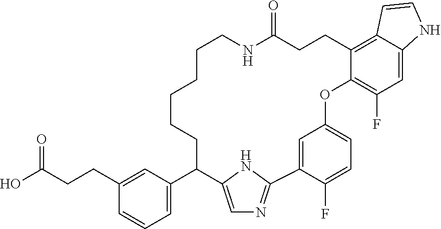 [SMILES: O=C(O)CCc1cccc(C2CCCCCCNC(=O)CCc3c(c(F)cc4[nH]ccc34)Oc3ccc(F)c(c3)-c3ncc2[nH]3)c1]

Exchanging 8-bromo-6-(3-iodophenyl)-7-oxooctanenitrile (Intermediate 1-1) with 9-bromo-7-(3-iodophenyl)-8-oxononanenitrile (intermediate 1-2, 0.25 g, 0.6 mmol), the reaction procedure sequence (Steps A to G) described for Example 1 was used to prepare the title compound (45 mg) as a white solid. MS (ESI): 627.3 m/z [M+H]⁺, retention time: 1.87 minutes, purity: 97% (254 nm) (LC-MS method 6).

### Example 5. Compound 5. 3-[3-(24,30-Difluoro-14-oxo-26-oxa-3,13,19,21,32-pentazapentacyclo-[25.3.1.12,5.017,25.018,22]dotriaconta-1(31),2,4,17,19,22,24,27,29-nonaen-6-yl)phenyl]propanoic acid

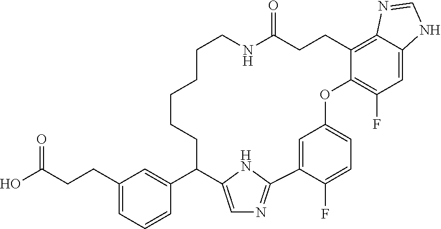 [SMILES: O=C(O)CCc1cccc(C2CCCCCCNC(=O)CCc3c(c(F)cc4[nH]cnc34)Oc3ccc(F)c(c3)-c3ncc2[nH]3)c1]

Exchanging methyl (E)-3-(5-(3-carbamimidoyl-4-fluorophenoxy)-6-fluoro-1H-indol-4-yl)acrylate (Intermediate 5) with benzyl (E)-3-(5-(3-carbamimidoyl-4-fluorophenoxy)-6-fluoro-1H-benzo[d]imidazol-4-yl)acrylate (Intermediate 5-13, 710 mg, 1.58 mmol), and 8-bromo-6-(3-iodophenyl)-7-oxooctanenitrile (Intermediate 1-1) with ethyl 3-(3-(1-bromo-8-cyano-2-oxooctan-3-yl)phenyl)propanoate (intermediate 1-37, 538 mg, 1.37 mmol), the reaction procedure sequence (Steps A, F, D, E and G, in this order) described for Example 1 was used to prepare the title compound as a white solid (10 mg). MS (ESI): 628.3 m/z [M+H]⁺, retention time: 1.53 minutes, purity: >99% (254 nm) (LC-MS method 7).

### Example 6. Compound 6. 3-[3-(23,29-Difluoro-14-oxo-25-oxa-3,13,20,31-tetrazapentacyclo-[24.3.1.12,5.016,24.017,21]hentriaconta-1(30),2,4,16,18,21,23,26,28-nonaen-6-yl)phenyl]propanoic acid

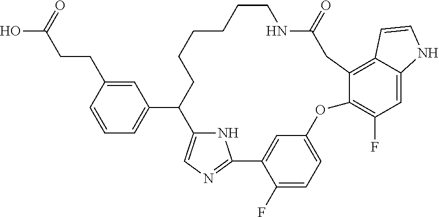 [SMILES: O=C(O)CCc1cccc(C2CCCCCCNC(=O)Cc3c(c(F)cc4[nH]ccc34)Oc3ccc(F)c(c3)-c3ncc2[nH]3)c1]

Exchanging methyl (E)-3-(5-(3-carbamimidoyl-4-fluorophenoxy)-6-fluoro-1H-indol-4-yl)acrylate (Intermediate 5) with methyl 2-(5-(3-carbamimidoyl-4-fluorophenoxy)-6-fluoro-1H-indol-4-yl)acetate (Intermediate 6-1, 390 mg, 1.09 mmol) and 8-bromo-6-(3-iodophenyl)-7-oxooctanenitrile (Intermediate 1-1) with 9-bromo-7-(3-iodophenyl)-8-oxononanenitrile (Intermediate 1-2, 516 mg, 1.19 mmol), the reaction procedure sequence (Steps A, B, C, D, E and G) described for Example 1 was used to prepare the title compound (14.9 mg). MS (ESI): 613 m/z [M+H]⁺, retention time: 1.64 minutes, purity: 98% (214 nm) (LC-MS method 7). ¹H NMR (400 MHz, CD₃OD) δ 7.70-7.66 (m, 1H), 7.34 (d, J=3.1 Hz, 1H), 7.26-7.18 (m, 5H), 7.06 (d, J=6.9 Hz, 1H), 6.98-6.91 (m, 1H), 6.88 (s, 1H), 6.52 (d, J=3.1 Hz, 1H), 4.01-3.95 (m, 1H), 3.82-3.75 (m, 2H), 3.17-2.96 (m, 2H), 2.89 (t, J=7.7 Hz, 2H), 2.57 (t, J=7.7 Hz, 2H), 2.23-2.11 (m, 1H), 1.87-1.83 (m, 1H), 1.54-1.23 (m, 8H) ppm.

### Example 7. Compound 7. 3-[3-(23,29-Difluoro-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]-2-methyl-propanoic acid

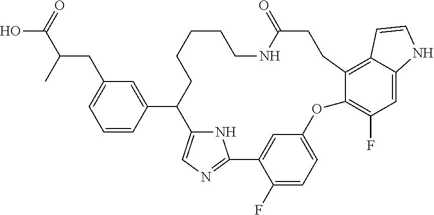 [SMILES: CC(Cc1cccc(C2CCCCCNC(=O)CCc3c(c(F)cc4[nH]ccc34)Oc3ccc(F)c(c3)-c3ncc2[nH]3)c1)C(=O)O]

Exchanging methyl (E)-3-(5-(3-carbamimidoyl-4-fluorophenoxy)-6-fluoro-1H-indol-4-yl)acrylate (Intermediate 5) with benzyl (E)-3-(5-(3-carbamimidoyl-4-fluorophenoxy)-6-fluoro-1H-indol-4-yl)acrylate (Intermediate 5-1, 1.0 g, 2.4 mmol) and 8-bromo-6-(3-iodophenyl)-7-oxooctanenitrile (Intermediate 1-1) with methyl (E)-3-(3-(1-bromo-7-cyano-2-oxoheptan-3-yl)phenyl)-2-methylacrylate (Intermediate 1-3, 0.78 g, 2 mmol), the reaction procedure sequence (Steps A, F, D, E and G, in this order) described for Example 1 was used to prepare the title compound (20.4 mg) as a white solid. MS (ESI): 627 m/z [M+H]⁺, retention time: 1.70 minutes, purity: 96% (254 nm) (LC-MS method 9). ¹H NMR (400 MHz, CD₃OD) δ 7.91-7.85 (m, 1H), 7.30 (d, J=3.3 Hz, 1H), 7.27-7.13 (m, 5H), 7.05 (s, 1H), 6.96 (d, J=9.0 Hz, 1H), 6.86 (s, 1H), 6.60 (d, J=3.2 Hz, 1H), 4.03 (d, J=6.6 Hz, 1H), 3.18-2.93 (m, 5H), 2.73-2.63 (m, 2H), 2.58 (t, J=8.2 Hz, 2H), 2.32-2.28 (m, 1H), 2.03-1.98 (m, 1H), 1.37-1.22 (m, 6H), 1.14 (d, J=5.8 Hz, 3H) ppm.

### Example 8. Compound 8. 3-[3-(21,27-Difluoro-11-oxo-23-oxa-3,10,18,29-tetrazapentacyclo[22.3.1.12,5.014,22.015,19]nonacosa-1(28),2,4,14,16,19,21,24,26-nonaen-6-yl)phenyl]propanoic acid

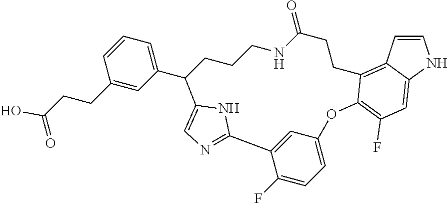 [SMILES: O=C(O)CCc1cccc(C2CCCNC(=O)CCc3c(c(F)cc4[nH]ccc34)Oc3ccc(F)c(c3)-c3ncc2[nH]3)c1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1 g, 2.6 mmol) and 8-bromo-6-(3-iodophenyl)-7-oxooctanenitrile (Intermediate 1-1) with 6-bromo-4-(3-iodophenyl)-5-oxohexanenitrile (Intermediate 1-4, 1 g, 2.6 mmol), the reaction procedure sequence (Steps A to G) described for Example 1 was used to prepare the title compound (29.6 mg) as a white solid. MS (ESI): 585 m/z [M+H]⁺, retention time: 1.64 minutes, purity: 80% (254 nm) (LC-MS method 4). ¹H NMR (400 MHz, CD₃OD) δ 7.43-7.37 (m, 1H), 7.32-7.11 (m, 5H), 7.05 (s, 2H), 6.98 (d, J=8.1 Hz, 1H), 6.64 (d, J=3.3 Hz, 1H), 6.60 (s, 1H), 3.85 (d, J=8.4 Hz, 1H), 3.01-2.97 (m, 2H), 2.92-2.80 (m, 2H), 2.55-2.36 (m, 4H), 2.16 (d, J=8.9 Hz, 1H), 1.87-1.83 (m, 1H), 1.66-1.62 (m, 1H), 1.36-1.30 (m, 3H) ppm.

### Example 9. Compound 9. 3-[3-(23,29-Difluoro-14-methyl-13-oxo-25-oxa-3,14,20,31-tetrazapentacyclo[24.3.1.12,5.016,24.017,21]hentriaconta-1(30),2,4,16(24),17(21),18,22,26,28-nonaen-6-yl)phenyl]propanoic acid

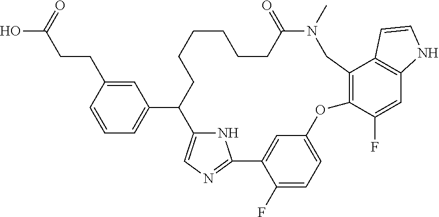 [SMILES: CN1Cc2c(c(F)cc3[nH]ccc23)Oc2ccc(F)c(c2)-c2ncc([nH]2)C(c2cccc(CCC(=O)O)c2)CCCCCCC1=O]

### 8-(2-(2-Fluoro-5-((6-fluoro-4-vinyl-1H-indol-5-yl)oxy)phenyl)-1H-imidazol-5-yl)-8-(3-iodophenyl)octanenitrile

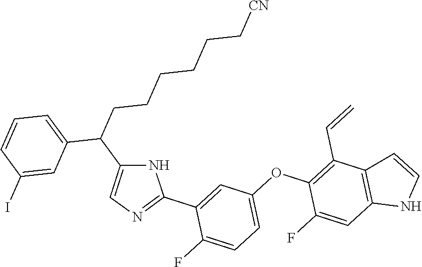 [SMILES: C=Cc1c(Oc2ccc(F)c(-c3ncc(C(CCCCCCC#N)c4cccc(I)c4)[nH]3)c2)c(F)cc2[nH]ccc12]

Step A: To a stirred solution of 10-bromo-8-(3-iodophenyl)-9-oxodecanenitrile (Intermediate 1-5, 2 g, 4.5 mmol) in N,N-dimethylformamide (20 mL) was added 2-fluoro-5-((6-fluoro-4-vinyl-1H-indol-5-yl)oxy)benzimidamide (Intermediate 5-3, 1.4 g, 4.5 mmol) and sodium bicarbonate (756 mg, 9.0 mmol). The reaction mixture was heated at 75° C. overnight, cooled to room temperature, and diluted with ethyl acetate (60 mL). The solution was washed with water, brine, dried over sodium sulfate and concentrated. The residue was purified by automated flash chromatography (40 g silica gel column, eluting with 0-40% ethyl acetate in petroleum ether) to give the title compound (2 g, 69%) as yellow solid. MS (ESI): 663 m/z [M+H]⁺, retention time: 1.85 minutes, purity: 90% (254 nm) (LC-MS method 4).

### 8-(2-(2-Fluoro-5-((6-fluoro-4-formyl-1H-indol-5-yl)oxy)phenyl)-1H-imidazol-5-yl)-8-(3-iodophenyl)octanenitrile

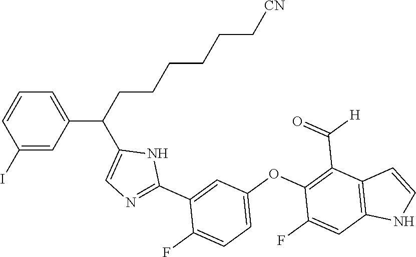 [SMILES: N#CCCCCCCC(c1cccc(I)c1)c1cnc(-c2cc(Oc3c(F)cc4[nH]ccc4c3C=O)ccc2F)[nH]1]

Step B: To a stirred solution of Step A product (1.5 g, 2.26 mmol) in tetrahydrofuran/water (30 mL) was added sodium periodate (1.5 g, 6.79 mmol), and 5 drops of osmium tetroxide (1% in water). The mixture was stirred at room temperature for 4 hours, quenched with saturated sodium dithionite, and extracted with ethyl acetate (3×60 mL). The combined organic layers were washed with brine, dried over sodium sulfate, and concentrated. The residue was purified by silica gel column chromatography (25 g silica gel column, eluting with petroleum ether: ethyl acetate=0˜80%) to give the title compound (1.1 g, 73%) as a yellow solid. MS (ESI): 665 m/z [M+H]⁺, retention time: 1.81 minutes, purity: 89% (254 nm) (LC-MS method 4).

### 8-(2-(2-Fluoro-5-((6-fluoro-4-((methylamino)methyl)-1H-indol-5-yl)oxy)phenyl)-1H-imidazol-5-yl)-8-(3-iodophenyl)octanenitrile

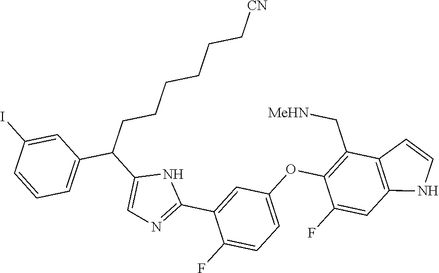 [SMILES: CNCc1c(Oc2ccc(F)c(-c3ncc(C(CCCCCCC#N)c4cccc(I)c4)[nH]3)c2)c(F)cc2[nH]ccc12]

Step C: To a stirred solution of Step B product (0.9 g, 1.35 mmol) in methanol (30 mL) was added methylamine (2.0 mL, 2 M in methanol, 4 mmol) and 3 drops of acetic acid. The mixture was stirred at room temperature for 1 hour, then treated with sodium cyanoborohydride (425 mg, 8.27 mmol) and stirred for an additional 16 hours. The mixture was quenched with water (100 mL), extracted with ethyl acetate (3×60 mL). The combined organic layers were washed with brine (20 mL), dried over sodium sulfate, and concentrated. The residue was purified by automated silica gel column chromatography (25 g column, eluting with petroleum ether: ethyl acetate=0˜80%) to give the title compound (480 mg, 43%) as a yellow solid. MS (ESI): 680 m/z [M+H]⁺, retention time: 1.75 minutes, purity: 90% (254 nm) (LC-MS method 4).

### 8-(2-(2-Fluoro-5-((6-fluoro-4-((methylamino)methyl)-1H-indol-5-yl)oxy)phenyl)-1H-imidazol-5-yl)-8-(3-iodophenyl)octanoic acid

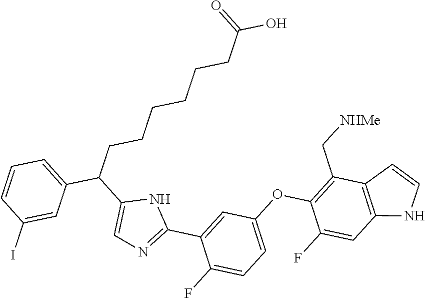 [SMILES: CNCc1c(Oc2ccc(F)c(-c3ncc(C(CCCCCCC(=O)O)c4cccc(I)c4)[nH]3)c2)c(F)cc2[nH]ccc12]

Step D: To a stirred solution of Step C product (0.48 g, 0.7 mmol) in ethanol (10 mL) and water (2 mL) was added potassium hydroxide (0.395 g, 7 mmol). The reaction was refluxed overnight, cooled to room temperature, and quenched with ice water. The solution was acidified to pH˜5-6 with 2 M hydrochloric acid, and extracted with ethyl acetate (3×40 mL). The combined organic layers were dried over sodium sulfate and concentrated to give the crude title compound (0.35 g, 71%) as a solid, which was used for next step without further purification. MS (ESI): 699 m/z [M+H]⁺, retention time: 1.45 minutes, purity: 72% (214 nm) (LC-MS Method 2).

### Compound 9: 3-[3-(23,29-Difluoro-14-methyl-13-oxo-25-oxa-3,14,20,31-tetrazapentacyclo[24.3.1.12,5.016,24.017,21]hentriaconta-1(30),2,4,16(24),17(21),18,22,26,28-nonaen-6-yl)phenyl]propanoic acid

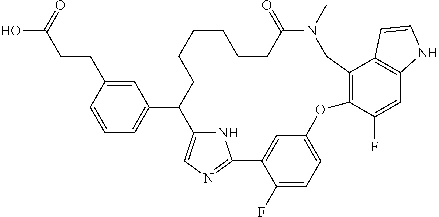 [SMILES: CN1Cc2c(c(F)cc3[nH]ccc23)Oc2ccc(F)c(c2)-c2ncc([nH]2)C(c2cccc(CCC(=O)O)c2)CCCCCCC1=O]

Exchanging the mixture of (E)-3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)acrylic acid and 3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)propanoic acid (Step D product of Example 1) with 8-(2-(2-fluoro-5-((6-fluoro-4-((methylamino)methyl)-1H-indol-5-yl)oxy)phenyl)-1H-imidazol-5-yl)-8-(3-iodophenyl)octanoic acid (Step D product of this Example, 320 mg, 0.480 mmol), the reaction procedure sequence (Steps E, B, F and G, in this order) described for Example 1 was used to prepare the title compound (10 mg) as a white solid. MS (ESI): 627 m/z [M+H]⁺, retention time: 2.85 minutes, purity: >99% (214 nm) (LC-MS method 10). ¹H NMR (400 MHz, CD₃OD) δ 7.72-7.59 (m, 1H), 7.39-7.03 (m, 7H), 6.92-6.62 (m, 3H), 5.20-5.03 (m, 1H), 4.74-4.56 (m, 1H), 3.99-3.91 (m, 1H), 3.36 (s, 3H), 2.93-2.89 (m, 2H), 2.61-2.62 (m, 5H), 1.84-1.22 (m, 9H) ppm.

### Example 10. Compound 10. 3-[3-(23,29-Difluoro-6,12-dimethyl-13-oxo-8,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

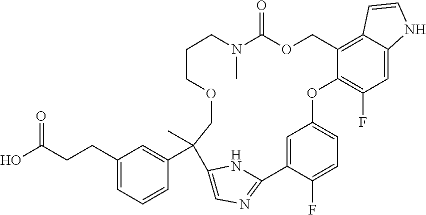 [SMILES: CN1CCCOCC(C)(c2cccc(CCC(=O)O)c2)c2cnc([nH]2)-c2cc(ccc2F)Oc2c(F)cc3[nH]ccc3c2COC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 700 mg, 1.9 mmol) and 8-bromo-6-(3-iodophenyl)-7-oxooctanenitrile (Intermediate 1-1) with benzyl (3-(4-bromo-2-(3-iodophenyl)-2-methyl-3-oxobutoxy)propyl)(methyl)carbamate (Intermediate 11, 1 g, 1.7 mmol), the reaction procedure sequence (Steps A, B, C, F, E, G, in this order) described for Example 1 was used to prepare the title compound (40 mg) as a white solid, MS (ESI): 643.3 m/z [M+H]⁺, retention time: 1.70 minutes, purity: >99% (214 nm) (LC-MS method 10).

### Example 11. Compound 11. 3-[3-(22,28-Difluoro-10-oxo-24-oxa-3,11,19,30-tetrazapentacyclo[23.3.1.12,5.015,23.016,20]triaconta-1(28),2,4,15(23),16(20),17,21,25(29),26-nonaen-6-yl)phenyl]propanoic acid

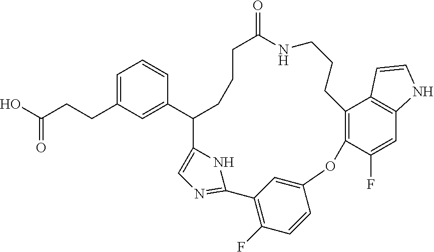 [SMILES: O=C(O)CCc1cccc(C2CCCC(=O)NCCCc3c(c(F)cc4[nH]ccc34)Oc3ccc(F)c(c3)-c3ncc2[nH]3)c1]

### 5-(2-(5-((4-(3-Azidopropyl)-6-fluoro-1H-indol-5-yl)oxy)-2-fluorophenyl)-1H-imidazol-5-yl)-5-(3-iodophenyl)pentanenitrile

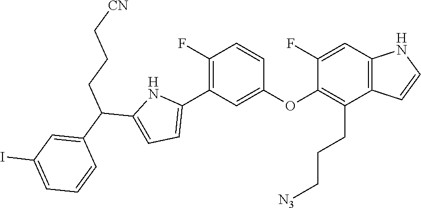 [SMILES: N#CCCCC(c1cccc(I)c1)c1ccc(-c2cc(Oc3c(F)cc4[nH]ccc4c3CCCN=[N+]=[N-])ccc2F)[nH]1]

Step A: To a stirred solution of 5-((4-(3-azidopropyl)-6-fluoro-1H-indol-5-yl)oxy)-2-fluorobenzimidamide (Intermediate 5-4, 1.2 g, 3.24 mmol) and 7-bromo-5-(3-iodophenyl)-6-oxoheptanenitrile (Intermediate 1, 1.1 g, 2.7 mmol) in N,N-dimethylformamide (30 mL) was added sodium bicarbonate (454 mg, 5.4 mmol). The mixture was stirred at 80° C. overnight, cooled to room temperature and quenched with water (100 mL). The solution was extracted with ethyl acetate (3×40 mL). The combined organic extracts were washed with brine (50 mL), dried over sodium sulfate, and concentrated. The residue was purified by automated flash chromatography (40 g silica gel column, eluting with 0-100% ethyl acetate in petroleum ether) to give the title compound (1.05 g, 57%) as a yellow solid. MS (ESI): 678 m/z [M+H]⁺, retention time: 1.98 minutes, purity: 95% (214 nm) (LC-MS method 3).

### 5-(2-(5-((4-(3-Azidopropyl)-6-fluoro-1H-indol-5-yl)oxy)-2-fluorophenyl)-1H-imidazol-5-yl)-5-(3-iodophenyl)pentanoic acid

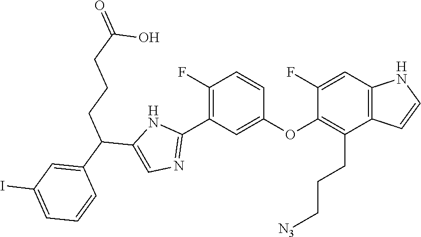 [SMILES: [N-]=[N+]=NCCCc1c(Oc2ccc(F)c(-c3ncc(C(CCCC(=O)O)c4cccc(I)c4)[nH]3)c2)c(F)cc2[nH]ccc12]

Step B: To a stirred solution of Step A product (1.05 g, 1.55 mmol) in ethanol (10 mL) and water (2 mL) was added potassium hydroxide (0.395 g, 7 mmol). The reaction was refluxed overnight, cooled to room temperature, and quenched with ice. The solution was acidified to pH 5-6 with 2 M hydrochloric acid and extracted with ethyl acetate (3×40 mL). The combined organic layers were dried over sodium sulfate and concentrated to give the crude title compound (1.07 g, crude) as a solid, which was used for next step without further purification. MS (ESI): 697 m/z [M+H]⁺.

### 5-(2-(5-((4-(3-Aminopropyl)-6-fluoro-1H-indol-5-yl)oxy)-2-fluorophenyl)-1H-imidazol-5-yl)-5-(3-iodophenyl)pentanoic acid

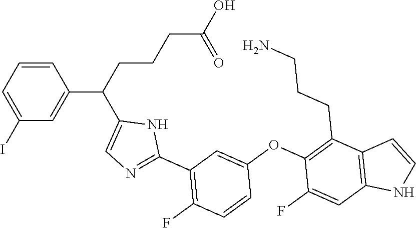 [SMILES: NCCCc1c(Oc2ccc(F)c(-c3ncc(C(CCCC(=O)O)c4cccc(I)c4)[nH]3)c2)c(F)cc2[nH]ccc12]

Step C: To a stirred solution of Step B product (1.07 g, crude) in tetrahydrofuran (18 mL) was added triphenylphosphine (805 mg, 3.08 mmol) and water (2 mL). The mixture was stirred at room temperature for 3 days and concentrated. The residue was dissolved in tetrahydrofuran (15 mL) and treated with concentrated ammonia (30%, 1.5 mL). The mixture was stirred at room temperature overnight and concentrated. The residue was dissolved in 50 mL of dichloromethane, stirred for 1 hour and filtered. The filter cake was rinsed with dichloromethane (20 mL) and dried in vacuo to give the title compound (0.66 g, 76% two steps) as a yellow solid. MS (ESI): 671 m/z [M+H]⁺, retention time: 1.40 minutes, purity: 74% (254 nm) (LC-MS method 11).

### Compound 11: 3-[3-(22,28-Difluoro-10-oxo-24-oxa-3,11,19,30-tetrazapentacyclo-[23.3.1.12,5.015,23.016,20]triaconta-1(28),2,4,15(23),16(20),17,21,25(29),26-nonaen-6-yl)phenyl]propanoic acid

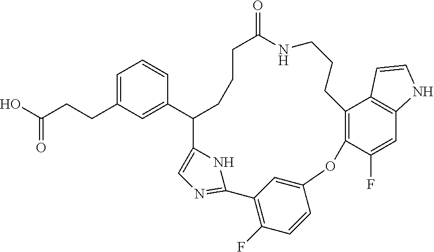 [SMILES: O=C(O)CCc1cccc(C2CCCC(=O)NCCCc3c(c(F)cc4[nH]ccc34)Oc3ccc(F)c(c3)-c3ncc2[nH]3)c1]

Step D: Exchanging the mixture of (E)-3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)acrylic acid and 3-(5-(3-(5-(6-amino-i-(3-(3-methoxy-3-oxopropyl)phenyl)hexyl)-1H-imidazol-2-yl)-4-fluorophenoxy)-6-fluoro-1H-indol-4-yl)propanoic acid (Step D product of Example) with 5-(2-(5-((4-(3-aminopropyl)-6-fluoro-1H-indol-5-yl)oxy)-2-fluorophenyl)-1H-imidazol-5-yl)-5-(3-iodophenyl)pentanoic acid (Step C product of this Example, 600 mg, 0.9 mmol, purity 74%), the reaction procedure sequence (Steps E, C, F, G, in this order) described for Example 1 was used to prepare the title compound (20.3 mg) as a white solid. MS (ESI): 599 m/z [M+H]+, retention time: 1.71 minutes, purity: 98% (254 nm) (LC-MS method 9). ¹H NMR (400 MHz, CD₃OD) δ 7.58-7.53 (m, 1H), 7.31 (d, J=3.2 Hz, 1H), 7.25-7.21 (m, 2H), 7.16-6.98 (m, 5H), 6.77 (s, 1H), 6.62 (d, J=3.3 Hz, 1H), 3.89-3.84 (m, 1H), 3.30-3.27 (m, 1H), 3.22-3.14 (m, 1H), 3.12-3.02 (m, 1H), 2.98-2.88 (m, 1H), 2.79-2.70 (m, 2H), 2.48 (t, J=7.7 Hz, 2H), 2.29-2.24 (m, 1H), 2.18-1.99 (m, 4H), 1.85-1.75 (m, 1H), 1.64-1.45 (m, 2H) ppm.

### Example 12. Compound 12. 3-[3-(23,29-Difluoro-6,10,10-trimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

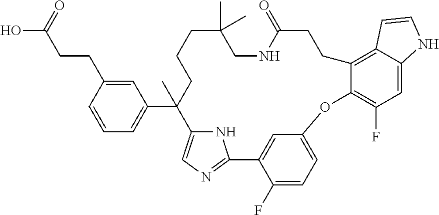 [SMILES: CC1(C)CCCC(C)(c2cccc(CCC(=O)O)c2)c2cnc([nH]2)-c2cc(ccc2F)Oc2c(F)cc3[nH]ccc3c2CCC(=O)NC1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1.1 g, 2.86 mmol) and 8-bromo-6-(3-iodophenyl)-7-oxooctanenitrile (Intermediate 1-1) with 8-bromo-6-(3-iodophenyl)-2,2,6-trimethyl-7-oxooctanenitrile (Intermediate 1-7, 1.31 g, 2.86 mmol), the reaction procedure sequence (Steps A, B, C, D, E and G, hydrogenation was complete at Step D so Step E was not needed) described for Example 1 was used to prepare the title compound (34 mg) as a white solid. MS (ESI): 655 m/z [M+H]⁺, retention time: 1.65 minutes, purity: 99% (214 nm) (LC-MS method 7). ¹H NMR (400 MHz, CD₃OD) δ7.92-7.90 (m, 1H), 7.28-7.03 (m, 8H), 6.62-6.60 (m, 2H), 3.21-3.18 (m, 2H), 2.90-2.87 (m, 2H), 2.75-2.06 (m, 8H), 1.61 (s, 3H), 1.42-0.92 (m, 4H), 0.82 (s, 3H), 0.62 (s, 3H) ppm.

### Example 13. Compound 13. 23,29-Difluoro-11,11-dioxo-6-phenyl-25-oxa-116-thia-3,12,20,31-tetrazapentacyclo[24.3.1.12,5.016,24.017,21]hentriaconta-1(30),2,4,16,18,21,23,26,28-nonaen-13-one

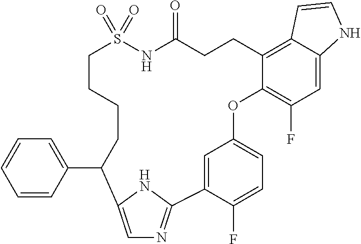 [SMILES: O=C1CCc2c(c(F)cc3[nH]ccc23)Oc2ccc(F)c(c2)-c2ncc([nH]2)C(c2ccccc2)CCCCS(=O)(=O)N1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 385 mg, 1.0 mmol) and 8-bromo-6-(3-iodophenyl)-7-oxooctanenitrile (Intermediate 1-1) with 7-bromo-6-oxo-5-phenylheptane-1-sulfonamide (Intermediate 20, 360 mg, 1.0 mmol)), the reaction procedure sequence (Steps A, F, B, E, in that order) described for Example 1 was used to prepare the title compound (33 mg) as a white solid. MS (ESI): 591 m/z [M+H]+, retention time: 1.75 minutes, purity: >99% (254 nm) (LC-MS method 4). ¹H NMR (400 MHz, CD₃OD) δ 7.95 (s, 1H), 7.39-7.35 (m, 2H), 7.30-7.26 (m, 3H), 7.19-7.15 (m, 2H), 7.13-7.06 (m, 1H), 6.93 (s, 1H), 6.73 (s, 1H), 6.53 (d, J=3.1 Hz, 1H), 4.05-4.01 (m, 1H), 3.01 (s, 3H), 2.75-2.65 (m, 2H), 2.35-2.31 (m, 1H), 2.10-1.92 (m, 2H), 1.74-1.60 (m, 2H), 1.31 (s, 2H) ppm.

### Example 14. Compound 14. 3-[3-(23,29-Difluoro-6,14-dimethyl-13-oxo-25-oxa-3,14,20,31-tetrazapentacyclo[24.3.1.12,5.016,24.017,21]hentriaconta-1(30),2,4,16(24),17(21),18,22,26,28-nonaen-6-yl)phenyl]propanoic acid

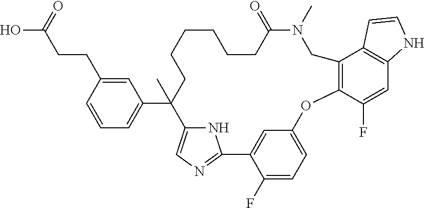 [SMILES: CN1Cc2c(c(F)cc3[nH]ccc23)Oc2ccc(F)c(c2)-c2ncc([nH]2)C(C)(c2cccc(CCC(=O)O)c2)CCCCCCC1=O]

### 8-(2-(2-Fluoro-5-((6-fluoro-4-(hydroxymethyl)-1H-indol-5-yl)oxy)phenyl)-1H-imidazol-5-yl)-8-(3-iodophenyl)nonanenitrile

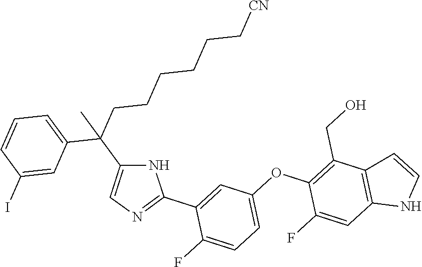 [SMILES: CC(CCCCCCC#N)(c1cccc(I)c1)c1cnc(-c2cc(Oc3c(F)cc4[nH]ccc4c3CO)ccc2F)[nH]1]

Step A: To a stirred solution of 10-bromo-8-(3-iodophenyl)-8-methyl-9-oxodecanenitrile (Intermediate 1-9, 857 mg, 1.85 mmol) in N,N-dimethylformamide (5 mL) was added 2-fluoro-5-((6-fluoro-4-(hydroxymethyl)-1H-indol-5-yl)oxy)benzimidamide (Intermediate 5-5, 588 mg, 1.85 mmol) and sodium bicarbonate (311 mg, 3.70 mmol). The reaction mixture was heated at 80° C. overnight, cooled to room temperature, and diluted with ethyl acetate (50 mL). The mixture was washed with water, brine, dried over sodium sulfate, and concentrated. The residue was purified by automated flash chromatography (40 g silica gel column, eluting with 0-40% ethyl acetate in petroleum ether) to give the title compound (800 mg, 63%) as a yellow solid. MS (ESI): 681 m/z [M+H]⁺, retention time: 1.87 minutes, purity: 90% (254 nm) (LC-MS method 4).

### 8-(2-(2-Fluoro-5-((6-fluoro-4-formyl-1H-indol-5-yl)oxy)phenyl)-1H-imidazol-5-yl)-8-(3-iodophenyl)nonanenitrile

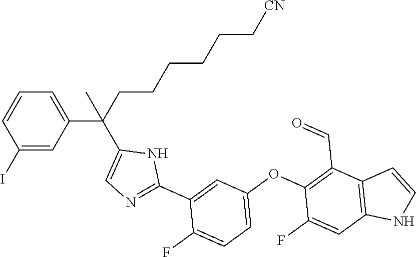 [SMILES: CC(CCCCCCC#N)(c1cccc(I)c1)c1cnc(-c2cc(Oc3c(F)cc4[nH]ccc4c3C=O)ccc2F)[nH]1]

Step B: to a stirred solution of Step A product (800 mg, 1.17 mmol) in tetrahydrofuran (30 mL) was added manganese dioxide (2 g, 23.4 mmol). The mixture was stirred at room temperature for 24 hours and filtered through a pad of Celite. The filter cake was washed with ethyl acetate (60 mL). The combined filtrate was concentrated. The residue was purified by automated silica gel column (25 g silica gel column, petroleum ether: ethyl acetate=0˜80%) to give the title compound (600 mg, 75%) as a yellow solid. MS (ESI): 679 m/z [M+H]+, retention time: 1.81 minutes, purity: 90% (254 nm) (LC-MS method 4).

### Compound 14. 3-[3-(23,29-Difluoro-6,14-dimethyl-13-oxo-25-oxa-3,14,20,31-tetrazapentacyclo[24.3.1.12,5.016,24.017,21]hentriaconta-1(30),2,4,16(24),]7(21),18,22,26,28-nonaen-6-yl)phenyl]propanoic acid

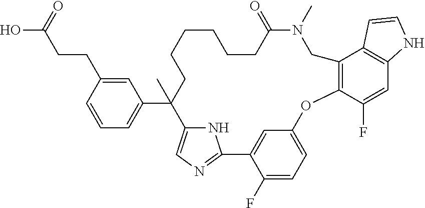 [SMILES: CN1Cc2c(c(F)cc3[nH]ccc23)Oc2ccc(F)c(c2)-c2ncc([nH]2)C(C)(c2cccc(CCC(=O)O)c2)CCCCCCC1=O]

Step C: Exchanging 8-(2-(2-Fluoro-5-((6-fluoro-4-formyl-1H-indol-5-yl)oxy)phenyl)-1H-imidazol-5-yl)-8-(3-iodophenyl)octanenitrile (Step B product of Example 9) with 8-(2-(2-Fluoro-5-((6-fluoro-4-formyl-1H-indol-5-yl)oxy)phenyl)-1H-imidazol-5-yl)-8-(3-iodophenyl)nonanenitrile (Step B product of this Example, 600 mg, 0.88 mmol), the reaction procedure sequence (Steps C and Step D of Example 9, followed by Steps E, C, F and G of Example 1, in this order) was used to prepare the title compound (3.5 mg) as a white solid. MS (ESI): 641.3 m/z [M+H]⁺, retention time: 1.64 minutes, purity: 96% (254 nm) (LC-MS method 4).

### Example 15. Compound 15. 3-[3-(9,9,22,28-Tetrafluoro-12-oxo-24-oxa-3,11,19,30-tetrazapentacyclo[23.3.1.12,5.015,23.016,20]triaconta-1(28),2,4,15(23),16(20),17,21,25(29),26-nonaen-6-yl)phenyl]propanoic acid

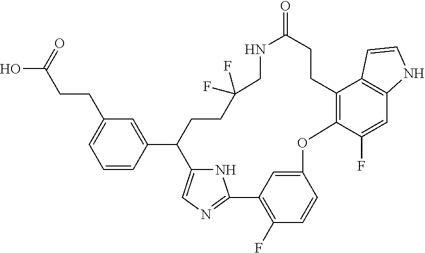 [SMILES: O=C(O)CCc1cccc(C2CCC(F)(F)CNC(=O)CCc3c(c(F)cc4[nH]ccc34)Oc3ccc(F)c(c3)-c3ncc2[nH]3)c1]

### Ethyl 3-(5-(3-(5-(5-azido-4,4-difluoro-1-(3-iodophenyl)pentyl)-1H-imidazol-2-yl)-4-fluorophenoxy)-6-fluoro-1H-indol-4-yl)propanoate

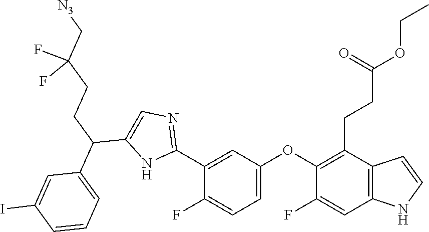 [SMILES: CCOC(=O)CCc1c(Oc2ccc(F)c(-c3ncc(C(CCC(F)(F)CN=[N+]=[N-])c4cccc(I)c4)[nH]3)c2)c(F)cc2[nH]ccc12]

Step A: To a stirred solution of ethyl 3-(5-(3-carbamimidoyl-4-fluorophenoxy)-6-fluoro-1H-indol-4-yl)propanoate (Intermediate 21, 0.96 g, 2.4 mmol), 7-azido-1-bromo-6,6-difluoro-3-(3-iodophenyl)heptan-2-one (Intermediate 1-10, 1.2 g, 2.4 mmol) in N,N-dimethylformamide (20 mL) was added sodium bicarbonate (0.4 g, 4.8 mmol). The mixture was heated at 80° C. overnight, cooled to room temperature and diluted with ethyl acetate (120 mL). The solution was washed with water (3×30 mL), brine (30 mL), dried over sodium sulfate, and concentrated. The residue was purified by automated silica gel column chromatography (40 g silica gel column; eluting with 0-60% ethyl acetate in petroleum ether) to give the title compound (0.4 g, 22%) as a solid. MS (ESI): 761 m/z [M+H]⁺, retention time: 1.92 minutes, purity: 80% (254 nm) (LC-MS method 7).

### 3-(5-(3-(5-(5-Azido-4,4-difluoro-1-(3-iodophenyl)pentyl)-1H-imidazol-2-yl)-4-fluorophenoxy)-6-fluoro-1H-indol-4-yl)propanoic acid

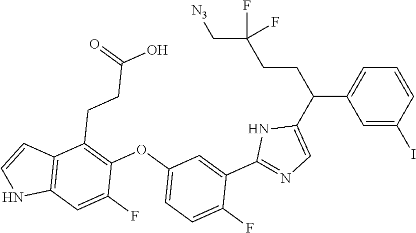 [SMILES: [N-]=[N+]=NCC(F)(F)CCC(c1cccc(I)c1)c1cnc(-c2cc(Oc3c(F)cc4[nH]ccc4c3CCC(=O)O)ccc2F)[nH]1]

Step B: To a stirred solution of Step A product (0.54 g, 0.7 mmol) in methanol (3 mL) and tetrahydrofuran (9 mL) was added lithium hydroxide monohydrate (1M in water, 2.1 mL). The mixture was stirred at room temperature for 16 hours, acidified with 1 M hydrochloric acid to pH˜4. The solution was extracted with ethyl acetate (2×50 mL). The combined organic extracts were washed with water, brine, dried over sodium sulfate, and concentrated to give the crude title compound (0.48 g, crude) as a solid. MS (ESI): 733 m/z [M+H]⁺, retention time: 1.80 minutes, purity: 95% (254 nm) (LC-MS method 7).

### Compound 15: 3-[3-(9,9,22,28-Tetrafluoro-12-oxo-24-oxa-3,11,19,30-tetrazapentacyclo[23.3.1.12,5.015,23.016,20]triaconta-1(28),2,4,15(23),16(20),17,21,25(29),26-nonaen-6-yl)phenyl]propanoic acid

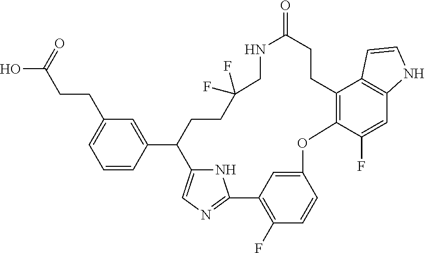 [SMILES: O=C(O)CCc1cccc(C2CCC(F)(F)CNC(=O)CCc3c(c(F)cc4[nH]ccc34)Oc3ccc(F)c(c3)-c3ncc2[nH]3)c1]

Step C: Exchanging 5-(2-(5-((4-(3-azidopropyl)-6-fluoro-1H-indol-5-yl)oxy)-2-fluorophenyl)-1H-imidazol-5-yl)-5-(3-iodophenyl)pentanoic acid (Step B product of Example 11) with 3-(5-(3-(5-(5-azido-4,4-difluoro-1-(3-iodophenyl)pentyl)-1H-imidazol-2-yl)-4-fluorophenoxy)-6-fluoro-1H-indol-4-yl)propanoic acid (Step B product of this Example, 0.38 g; 0.52 mmol), the reaction procedure sequence (Steps C of Example 11, followed by Steps E, C, F and G of Example 1) was used to prepare the title compound (17.5 mg) as a white solid. MS (ESI): 635 m/z [M+H]⁺, retention time: 1.66 minutes, purity: 96% (214 nm) (LC-MS method 7). ¹H NMR (400 MHz, CD₃OD) 7.71-7.74 (m, 1H), 7.19-7.28 (m, 3H), 7.12-7.17 (m, 2H), 7.07-7.11 (m, 2H), 7.06-7.02 (m, 1H), 6.77 (s, 1H), 6.58-6.54 (m, 1H), 4.01-3.95 (d, 1H), 3.58-3.64 (m, 1H), 3.21-3.31 (m, 3H), 2.92-2.86 (m, 2H), 2.53-2.60 (m, 3H), 2.43-2.48 (m, 1H), 2.15-2.27 (m, 2H), 1.93-2.00 (m, 2H) ppm.

### Example 16. Compound 16. 3-[3-(22,28-Difluoro-6-methyl-11-oxo-24-oxa-3,12,19,30-tetrazapentacyclo[23.3.1.12,5.015,23.016,20]triaconta-1(29),2,4,15,17,20,22,25,27-nonaen-6-yl)phenyl]propanoic acid

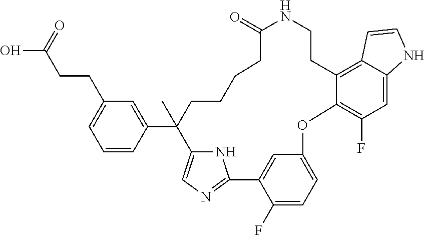 [SMILES: CC1(c2cccc(CCC(=O)O)c2)CCCCC(=O)NCCc2c(c(F)cc3[nH]ccc23)Oc2ccc(F)c(c2)-c2ncc1[nH]2]

Exchanging 5-((4-(3-azidopropyl)-6-fluoro-1H-indol-5-yl)oxy)-2-fluorobenzimidamide (Intermediate 5-4) with 5-((4-(2-azidoethyl)-6-fluoro-1H-indol-5-yl)oxy)-2-fluorobenzimidamide (Intermediate 5-6, 1.0 g, 2.81 mmol), and 7-bromo-5-(3-iodophenyl)-6-oxoheptanenitrile (Intermediate 1) with 8-bromo-6-(3-iodophenyl)-6-methyl-7-oxooctanenitrile (Intermediate 1-11, 1.2 g, 2.81 mmol), the reaction procedure sequence (Steps A to C of Example 11, followed by Steps E, C, F, and G of Example 1, in this order) was used to prepare the title compound (44 mg) as a white solid. MS (ESI): 613 m/z [M+H]⁺. ¹H NMR (400 MHz, CD₃OD) δ 7.63-7.60 (m, 1H), 7.29-7.27 (m, 2H), 7.24-7.20 (m, 1H), 7.17-7.10 (m, 2H), 7.05 (s, 1H), 7.02 (s, 1H), 7.00-6.96 (m, 1H), 6.93-6.89 (m, 1H), 6.53-6.49 (m, 1H), 3.78-3.75 (m, 1H), 3.32-3.15 (m, 1H), 3.07-3.03 (m, 2H), 2.85-2.81 (m, 2H), 2.53-2.47 (m, 2H), 2.41-2.35 (m, 1H), 2.11-2.07 (m, 2H), 1.77-1.74 (m, 2H), 1.54 (s, 3H), 1.35-1.31 (m, 3H) ppm.

### Example 17. Compound 17. 3-[3-(22,28-Difluoro-6-methyl-10-oxo-24-oxa-3,11,19,30-tetrazapentacyclo[23.3.1.12,5.015,23.016,20]triaconta-1(29),2,4,15,17,20,22,25,27-nonaen-6-yl)phenyl]propanoic acid

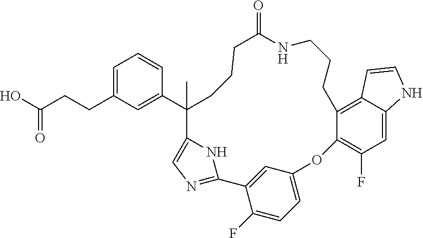 [SMILES: CC1(c2cccc(CCC(=O)O)c2)CCCC(=O)NCCCc2c(c(F)cc3[nH]ccc23)Oc2ccc(F)c(c2)-c2ncc1[nH]2]

Exchanging 7-bromo-5-(3-iodophenyl)-6-oxoheptanenitrile (Intermediate 1) with 7-bromo-5-(3-iodophenyl)-5-methyl-6-oxoheptanenitrile (Intermediate 1-12, 1.6 g, 3.78 mmol), the reaction procedure sequence (Steps A to C of Example 11, followed by Steps E, C, F, and G of Example 1) was used to prepare the title compound (45 mg) as a white solid. MS (ESI): 613 m/z [M+H]⁺. ¹H NMR (400 MHz, CD₃OD) δ 7.47-7.43 (m, 1H), 7.28-7.24 (m, 1H), 7.24-7.21 (m, 2H), 7.14 (s, 1H), 7.09-7.01 (m, 3H), 6.98 (s, 1H), 6.95-6.94 (m, 1H), 6.58-6.54 (m, 1H), 3.3.23-3.20 (m, 1H), 3.11-3.06 (m, 2H), 2.87-2.84 (m, 1H), 3.73-2.70 (m, 3H), 2.46-2.41 (m, 2H), 2.23-2.18 (m, 1H), 2.06-1.90 (m, 3H), 1.54 (s, 3H), 1.35-1.31 (m, 3H) ppm.

### Example 18. Compound 18. 3-[3-(23,29-Difluoro-6,12,14,14-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

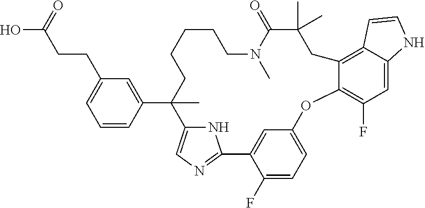 [SMILES: CN1CCCCCC(C)(c2cccc(CCC(=O)O)c2)c2cnc([nH]2)-c2cc(ccc2F)Oc2c(F)cc3[nH]ccc3c2CC(C)(C)C1=O]

Exchanging methyl (E)-3-(5-(3-carbamimidoyl-4-fluorophenoxy)-6-fluoro-1H-indol-4-yl)acrylate (Intermediate 5) with methyl 3-(5-(3-carbamimidoyl-4-fluorophenoxy)-6-fluoro-1H-indol-4-yl)-2,2-dimethylpropanoate (Intermediate 5-7, 626 mg, 1.56 mmol), and 8-bromo-6-(3-iodophenyl)-7-oxooctanenitrile (Intermediate 1-1) with benzyl (8-chloro-6-(3-iodophenyl)-6-methyl-7-oxooctyl)(methyl)carbamate (Intermediate 1-13, 845 mg, 1.56 mmol), the reaction procedure sequence (Steps A, B, C, F, E and G, in this order) described for Example 1 was used to prepare the title compound (29 ng) as a white solid. MS (ESI): 669 m/z [M+H]⁺, purity: 96% (214 nm) (LC-MS method 3). ¹H NMR (400 MHz, CD₃OD) δ 7.72 (s, 1H), 7.16-6.91 (m, 8H), 6.47 (s, 1H), 6.38 (d, J=2.8 Hz, 1H), 3.17-2.92 (m, 7H), 2.77 (t, J=8.0 Hz, 2H), 2.41 (t, J=8.0 Hz, 2H), 2.30-2.24 (m, 1H), 2.01-1.90 (m, 1H), 1.86 (s, 1H), 1.49-1.42 (m, 5H), 1.19-1.06 (m, 9H) ppm.

### Example 19. Compound 19. 3-[3-(22,28-Difluoro-6,11-dimethyl-12-oxo-8,24-dioxa-3,11,19,30-tetrazapentacyclo[23.3.1.12,5.015,23.016,20]triaconta-1(29),2,4,15,17,20,22,25,27-nonaen-6-yl)phenyl]propanoic acid

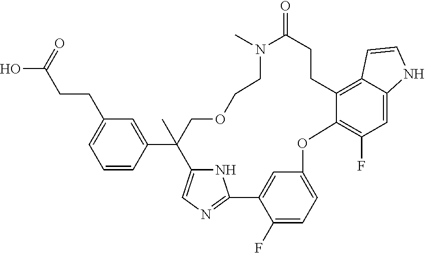 [SMILES: CN1CCOCC(C)(c2cccc(CCC(=O)O)c2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 728 mg, 1.89 mmol), and 8-bromo-6-(3-iodophenyl)-7-oxooctanenitrile (Intermediate 1-1) with benzyl (2-(4-bromo-2-(3-iodophenyl)-2-methyl-3-oxobutoxy)ethyl)(methyl)carbamate (Intermediate 11-2, 990 mg, 1.72 mmol), the 6 step reaction procedure sequence (Steps A, B, C, F, E and G, in this order) described for Example 1 was used to prepare the title compound (34.5 mg) as a white solid. MS (ESI): 629 m/z [M+H]⁺. ¹H NMR (400 MHz, CD₃OD) δ 7.52-7.50 (m, 1H), 7.35-7.04 (m, 9H), 6.63-6.48 (m, 1H), 4.07 (d, J=8.8 Hz, 1H), 3.77 (d, J=8.8 Hz, 1H), 3.68-3.62 (m, 1H), 3.50-3.36 (m, 1H), 3.25-3.18 (m, 4H), 2.85-2.23 (m, 9H), 1.67-1.57 (m, 3H) ppm

### Example 20. Compound 20A (racemic), and its separated enantiomers, Example 20B and 20C, Enantiomers 1 and 2 of 3-[3-(23,29-Difluoro-6,10,10,12-tetramethyl-13-oxo-8,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

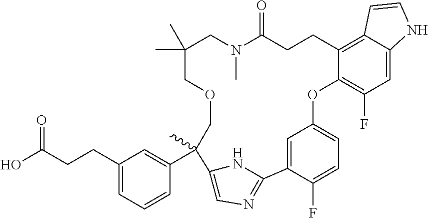 [SMILES: CN1CC(C)(C)COCC(C)(c2cccc(CCC(=O)O)c2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1.1 g, 2.93 mmol), and 8-bromo-6-(3-iodophenyl)-7-oxooctanenitrile (Intermediate 1-1) with benzyl (3-(4-bromo-2-(3-iodophenyl)-2-methyl-3-oxobutoxy)-2,2-dimethylpropyl)(methyl)carbamate (Intermediate 11-3, 1.8 g, 2.93 mmol), the 6 step reaction procedure sequence (Steps A, B, C, F, E and G, in this order) described for Example 1 was used to prepare racemic Example 20A (40 mg) as a white solid. MS (ESI): 671 m/z [M+H]⁺.

In another scale up run, 1.80 g of racemic ethyl ester of 20A (corresponding Step E product of Example 1) was obtained. This ethyl ester was subject to chiral SFC separation under the following conditions: Instrument: SFC-80 (Thar, Waters); Column: SC 20*250 mm, 10 μm; Column temperature: 35° C.; Mobile phase: carbon dioxide/methanol (0.2% methanol ammonia)=60/40; Flow rate: 80 g/minute; Back pressure: 100 bar; Detection wavelength: 214 nm; Cycle time: 6 minutes; Sample solution: 1800 mg dissolved in 40 mL methanol; Injection volume: 1 mL. The first eluent (0.58 g, 35%), Enantiomer 1, was further hydrolyzed to 20B (508 mg, 91%, white solid); The second eluent (0.62 g, 38%), Enantiomer 2, was further hydrolyzed to 20C (535 mg, 90%, white solid), following the conditions described in Step G of Example 1. Compound 20B: MS (ESI): 671 m/z [M+H]⁺, retention time: 1.37 minutes, purity: 97% (214 nm) (LC-MS method 17). ¹H NMR (400 MHz, CD₃OD) δ 7.50-7.46 (m, 1H), 7.40-7.28 (m, 4H), 7.27-7.21 (m, 1H), 7.18-7.08 (m, 4H), 6.57-6.53 (m, 1H), 4.06-4.02 (m, 1H), 3.75 (d, J=9.1 Hz, 1H), 3.30-3.25 (m, 1H), 3.23-3.02 (m, 7H), 2.94 (d, J=13.5 Hz, 1H), 2.85 (t, J=7.0 Hz, 2H), 2.79-2.75 (m, 2H), 2.56-2.52 (m, 2H), 1.72 (s, 3H), 0.78-0.62 (m, 6H) ppm

Compound 20C: MS (ESI): 671 m/z [M+H]⁺, retention time: 1.38 minutes, purity: >99% (214 nm) (LC-MS method 17). ¹H NMR (400 MHz, CD₃OD) δ 7.50-7.46 (m, 1H), 7.39-7.28 (m, 4H), 7.27-7.22 (m, 1H), 7.19-7.07 (m, 4H), 6.57-6.53 (m, 1H), 4.06-4.02 (m, 1H), 3.75 (d, J=9.1 Hz, 1H), 3.30-3.25 (m, 1H), 3.20-3.01 (m, 7H), 2.94 (d, J=13.7 Hz, 1H), 2.85 (t, J=7.1 Hz, 2H), 2.79-2.74 (m, 2H), 2.56-2.52 (m, 2H), 1.72 (s, 3H), 0.77-0.63 (m, 6H) ppm

### Example 21. Compound 21. 3-[3-(23,29-Difluoro-12-methyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]butanoic acid

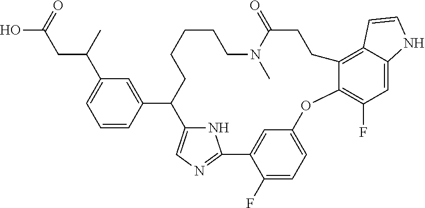 [SMILES: CC(CC(=O)O)c1cccc(C2CCCCCN(C)C(=O)CCc3c(c(F)cc4[nH]ccc34)Oc3ccc(F)c(c3)-c3ncc2[nH]3)c1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 2.41 g, 6.26 mmol), 8-bromo-6-(3-iodophenyl)-7-oxooctanenitrile (Intermediate 1-1) with benzyl (8-chloro-6-(3-iodophenyl)-6-methyl-7-oxooctyl)(methyl)carbamate (Intermediate 1-13, 3.3 g, 6.26 mmol) in corresponding Step A, and methyl acrylate with methyl (E)-but-2-enoate (300 mg, 3.0 mmol) in corresponding Step C, the reaction procedure sequence (Steps A, B, C, F, E and G, in this order) described for Example 1 was used to prepare the title compound (42 mg, 500 mg out of 1.2 g of corresponding Step B product was used for following corresponding Step C and after) as a white solid. MS (ESI): 641 m/z [M+H]⁺. ¹HNMR (400 MHz, CD₃OD) δ 7.42-7.04 (m, 9H), 6.75 (s, 1H), 6.56-6.51 (m, 1H), 3.92-3.87 (m, 1H), 3.30-2.48 (m, 12H), 1.99-1.77 (m, 2H), 1.43-1.23 (m, 9H) ppm.

### Example 22. Compound 22. 3-[3-(23,29-Difluoro-12-methyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]-2-methyl-propanoic acid

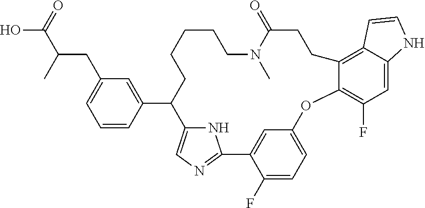 [SMILES: CC(Cc1cccc(C2CCCCCN(C)C(=O)CCc3c(c(F)cc4[nH]ccc34)Oc3ccc(F)c(c3)-c3ncc2[nH]3)c1)C(=O)O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 2.41 g. 6.26 mmol), 8-bromo-6-(3-iodophenyl)-7-oxooctanenitrile (Intermediate 1-1) with benzyl (8-chloro-6-(3-iodophenyl)-6-methyl-7-oxooctyl)(methyl)carbamate (Intermediate 1-13, 3.3 g, 6.26 mmol) in Step A. and methyl acrylate with methyl methacrylate (300 mg, 3.0 mmol) in Step C, the reaction procedure sequence (Steps A, B, C, F, E and G, in this order) described for Example 1 was used to prepare the title compound (72 mg, 500 mg out of 1.2 g of corresponding Step B product was used for corresponding Step C and after) as a white solid. MS (ESI): 641 m/z [M+H]⁺. ¹H NMR (400 MHz, CD₃OD) δ 7.40-7.01 (m, 9H), 6.95-6.78 (m, 1H), 6.54-6.51 (m, 1H), 4.05-3.88 (m, 1H), 3.30-2.59 (m, 12H), 1.97-1.92 (m, 1H), 1.82-1.59 (m, 1H), 1.42-0.92 (m, 9H) ppm.

### Example 23. Compound 23. 3-[3-(23,29-Difluoro-6,10-dimethyl-13-oxo-8,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

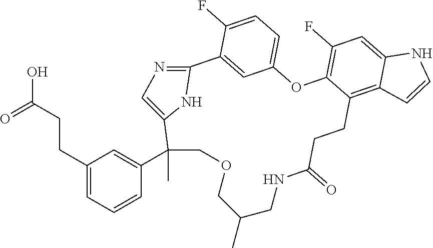 [SMILES: CC1CNC(=O)CCc2c(c(F)cc3[nH]ccc23)Oc2ccc(F)c(c2)-c2ncc([nH]2)C(C)(c2cccc(CCC(=O)O)c2)COC1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 0.92 g, 2.4 mmol), and 8-bromo-6-(3-iodophenyl)-7-oxooctanenitrile (Intermediate 1-1) with benzyl (3-(4-bromo-2-(3-iodophenyl)-2-methyl-3-oxobutoxy)-2-methylpropyl)carbamate (Intermediate 11-4, 1.4 g, 2.4 mmol) in Step A, the 6 step reaction procedure sequence (Steps A, B, C, F, E and G, in this order) described for Example 1 was used to prepare the title compound (24 mg) as a grey solid. MS (ESI): 643.3 m/z [M+H]⁺. (A mixture of 4 diastereomers, originated from two chiral centers).

### Example 24. Compound 24. 3-[3-(22,28-Difluoro-6-methyl-9-oxo-24-oxa-3,10,19,30-tetrazapentacyclo[23.3.1.12,5.015,23.016,20]triaconta-1(29),2,4,15,17,20,22,25,27-nonaen-6-yl)phenyl]propanoic acid

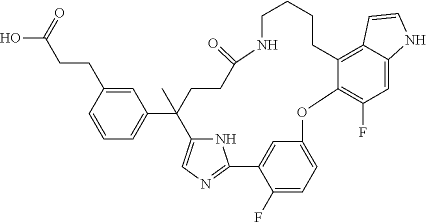 [SMILES: CC1(c2cccc(CCC(=O)O)c2)CCC(=O)NCCCCc2c(c(F)cc3[nH]ccc23)Oc2ccc(F)c(c2)-c2ncc1[nH]2]

### Example 25. Compound 25. 22,28-Difluoro-6-methyl-6-phenyl-24-oxa-3,10,19,30-tetrazapentacyclo[23.3.1.12,5.015,23.016,20]triaconta-1(29),2,4,15,17,20,22,25,27-nonaen-9-one

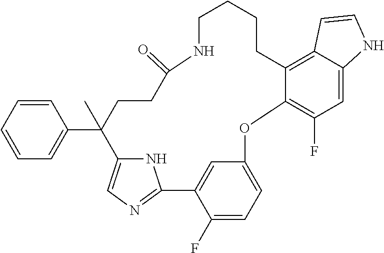 [SMILES: CC1(c2ccccc2)CCC(=O)NCCCCc2c(c(F)cc3[nH]ccc23)Oc2ccc(F)c(c2)-c2ncc1[nH]2]

Exchanging 5-((4-(3-azidopropyl)-6-fluoro-1H-indol-5-yl)oxy)-2-fluorobenzimidamide (Intermediate 5-4) with 5-((4-(4-azidobutyl)-6-fluoro-1H-indol-5-yl)oxy)-2-fluorobenzimidamide (Intermediate 5-8, 830 mg, 2.16 mmol), and 7-bromo-5-(3-iodophenyl)-6-oxoheptanenitrile (Intermediate 1) with 6-chloro-4-(3-iodophenyl)-4-methyl-5-oxohexanenitrile (Intermediate 1-14, 780 mg, 2.16 mmol), the reaction procedure sequence (Steps A to C of Example 11, followed by Steps E, C, F, and G of Example 1, in this order) was used to prepare the title compounds. 24 (6.9 mg) was obtained as a white solid. 25 (9.7 mg, white solid), the de-bromination product from Heck coupling, corresponding Step C of Example 1, was separated at the final step, corresponding Step G of Example 1.

Compound 24: MS (ESI): 613 m/z [M+H]⁺, retention time: 1.65 minutes, purity: >99% (214 nm) (LC-MS method 8). ¹H NMR (400 MHz, CD₃OD) δ 7.40-7.33 (m, 1H), 7.30-7.16 (m, 3H), 7.09-6.95 (m, 6H), 6.54 (d, J=3.1 Hz, 1H), 3.11-3.01 (m, 2H), 2.92-2.69 (m, 4H), 2.51-2.40 (m, 3H), 2.24-2.07 (m, 2H), 1.91-1.74 (m, 3H), 1.51 (s, 3H), 1.42-1.35 (m, 2H) ppm.

Compound 25: MS (ESI): 541 m/z [M+H]⁺, retention time: 1.71 minutes, purity: 98% (214 nm) (LC-MS method 8). ¹H NMR (400 MHz, CD₃OD) δ 7.76-7.54 (m, 1H), 7.42 (s, 1H), 7.29-7.03 (m, 9H), 6.54 (d, J=2.7 Hz, 1H), 3.15-3.05 (m, 2H), 2.96-2.79 (m, 2H), 2.50-2.43 (m, 1H), 2.30-2.08 (m, 2H), 1.92-1.67 (m, 3H), 1.56-1.48 (m, 3H), 1.34-1.26 (m, 2H) ppm.

### Example 26. Compound 26. 3-[3-(22,28-Difluoro-6-methyl-8-oxo-24-oxa-3,9,19,30-tetrazapentacyclo[23.3.1.12,5.015,23.016,20]triaconta-1(29),2,4,15,17,20,22,25,27-nonaen-6-yl)phenyl]propanoic acid

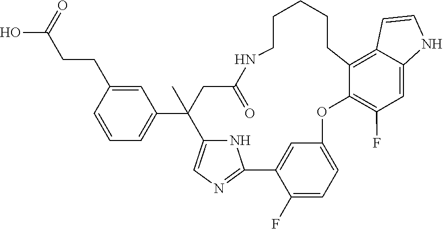 [SMILES: CC1(c2cccc(CCC(=O)O)c2)CC(=O)NCCCCCc2c(c(F)cc3[nH]ccc23)Oc2ccc(F)c(c2)-c2ncc1[nH]2]

### Example 27. Compound 27. 22,28-Difluoro-6-methyl-6-phenyl-24-oxa-3,9,19,30-tetrazapentacyclo[23.3.1.12,5.015,23.016,20]triaconta-1(29),2,4,15,17,20,22,25,27-nonaen-8-one

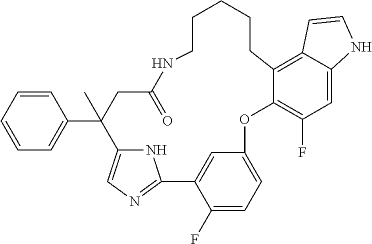 [SMILES: CC1(c2ccccc2)CC(=O)NCCCCCc2c(c(F)cc3[nH]ccc23)Oc2ccc(F)c(c2)-c2ncc1[nH]2]

Exchanging 5-((4-(3-azidopropyl)-6-fluoro-1H-indol-5-yl)oxy)-2-fluorobenzimidamide (Intermediate 5-4) with 5-((4-(5-azidopentyl)-6-fluoro-1H-indol-5-yl)oxy)-2-fluorobenzimidamide (Intermediate 5-9, 990 mg, 2.16 mmol), and 7-bromo-5-(3-iodophenyl)-6-oxoheptanenitrile (Intermediate 1) with 5-chloro-3-(3-iodophenyl)-3-methyl-4-oxopentanenitrile (Intermediate 1-15, 780 mg, 2.16 mmol), the reaction procedure sequence (Steps A to C of Example 11, followed by Steps E, C, F, and G of Example 1, in this order) was used to prepare the title compounds. 26 (9 mg) was obtained as a white solid. 27 (2.1 mg, white solid), the de-bromination product from Heck coupling, corresponding Step C of Example 1, was only separated at corresponding Step G of Example 1.

### Compound 26: MS (ESI): 613 m/z [M+H]⁺, retention time: 1.78 minutes, purity: >99% (214 nm) (LC-MS method 12). ¹H NMR (400 MHz, CD₃OD) δ 7.47-7.45 (m, 1H), 7.28-7.22 (m, 2H), 7.16-7.09 (m, 4H), 7.04-6.94 (m, 3H), 6.53 (d, J=3.2 Hz, 1H), 3.15-3.10 (m, 1H), 3.04-2.98 (m, 1H), 2.94-2.83 (m, 5H), 2.74-2.69 (m, 1H), 2.54-2.50 (m, 2H), 1.71 (s, 3H), 1.70-1.66 (m, 1H), 1.55-1.52 (m, 1H), 1.32-1.07 (m, 4H) ppm

Compound 27: MS (ESI): 541 m/z [M+H]⁺, retention time: 1.86 minutes, purity: >99% (214 nm) (LC-MS method 8). ¹H NMR (400 MHz, CD₃OD) δ 7.48-7.45 (m, 1H), 7.28-7.21 (m, 4H), 7.16-7.09 (m, 5H), 7.00 (s, 1H), 6.53 (d, J=3.2 Hz, 1H), 3.15-3.10 (m, 1H), 3.02-2.97 (m, 1H), 2.92-2.85 (m, 3H), 2.74-2.69 (m, 1H), 1.72 (s, 3H), 1.71-1.68 (m, 1H), 1.55-1.52 (m, 1H), 1.32-1.07 (m, 4H) ppm

### Example 28. Compound 28. 3-[3-(23,29-Difluoro-10,10,12-trimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)-2-fluoro-phenyl]propanoic acid

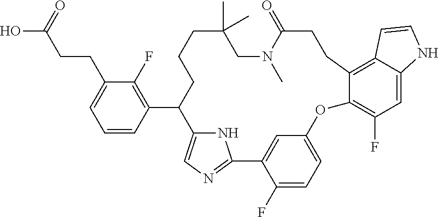 [SMILES: CN1CC(C)(C)CCCC(c2cccc(CCC(=O)O)c2F)c2cnc([nH]2)-c2cc(ccc2F)Oc2c(F)cc3[nH]ccc3c2CCC1=O]

Exchanging 8-bromo-6-(3-iodophenyl)-7-oxooctanenitrile (Intermediate 1-1) with benzyl (6-(3-bromo-2-fluorophenyl)-8-chloro-2,2-dimethyl-7-oxooctyl)(methyl)carbamate (Intermediate 1-16, 1 g, 1.9 mmol) in Step A, the 6 step reaction procedure sequence (Steps A, B, C, F, E and G, in this order) described for Example 1 was used to prepare the title compound (150 mg) as a grey solid. MS (ESI): 673 m/z [M+H]⁺; retention time: 1.86 minutes, purity: 98% (214 nm) (LC-MS method 5). ¹H NMR (400 MHz, CD₃OD) δ 8.12-6.79 (m, 9H), 6.59-6.45 (m, 1H), 4.44-4.25 (m, 1H), 3.27-2.53 (m, 13H), 1.99-1.74 (m, 2H), 1.47-0.98 (m, 4H), 0.85-0.69 (m, 6H) ppm.

### Example 29. Compound 29. 3-[3-(23,29-Difluoro-6,9,9,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

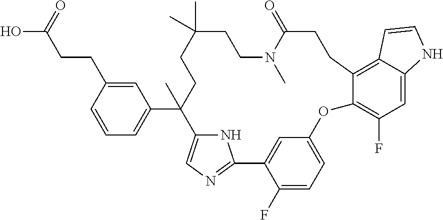 [SMILES: CN1CCC(C)(C)CCC(C)(c2cccc(CCC(=O)O)c2)c2cnc([nH]2)-c2cc(ccc2F)Oc2c(F)cc3[nH]ccc3c2CCC1=O]

Exchanging 8-bromo-6-(3-iodophenyl)-7-oxooctanenitrile (Intermediate 1-1) with benzyl (8-bromo-6-(3-iodophenyl)-3,3,6-trimethyl-7-oxooctyl)(methyl)carbamate (Intermediate 1-19, 560 mg, 0.916 mmol), the reaction procedure sequence (Steps A, B, C, F, E and G, in this order) described for Example 1 was used to prepare the title compound (40 mg) as a white solid. MS (ESI): 669 m/z [M+H]⁺, retention time: 1.86 minutes, purity: 96% (254 nm) (LC-MS method 14).

¹H NMR (400 MHz, CD₃OD) δ 7.71-7.69 (m, 1H), 7.36-6.93 (m, 9H), 6.54-6.50 (m, 1H), 3.60-3.30 (m, 1H), 3.20-2.70 (m, 9H), 2.61-2.55 (m, 1H), 2.49-2.43 (m, 1H), 2.36-2.05 (m, 2H), 1.80-1.49 (m, 4H), 1.36-0.79 (m, 10H).

### Example 30. Compound 30. 3-[3-(23,29-Difluoro-9,9,12-trimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)-2-fluoro-phenyl]propanoic acid

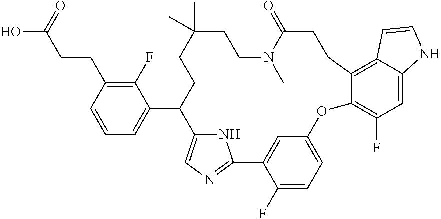 [SMILES: CN1CCC(C)(C)CCC(c2cccc(CCC(=O)O)c2F)c2cnc([nH]2)-c2cc(ccc2F)Oc2c(F)cc3[nH]ccc3c2CCC1=O]

### Example 31. Compound 31. 23,29-difluoro-6-(2-fluorophenyl)-9,9,12-trimethyl-25-oxa-3,12,20,31-tetrazapentacyclo[24.3.1.12,5.016,24.017,21]hentriaconta-1(30),2,4,16,18,21,23,26,28-nonaen-13-one

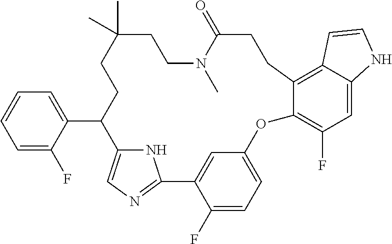 [SMILES: CN1CCC(C)(C)CCC(c2ccccc2F)c2cnc([nH]2)-c2cc(ccc2F)Oc2c(F)cc3[nH]ccc3c2CCC1=O]

Exchanging 8-bromo-6-(3-iodophenyl)-7-oxooctanenitrile (Intermediate 1-1) with benzyl (8-bromo-6-(3-bromo-2-fluorophenyl)-3,3-dimethyl-7-oxooctyl)(methyl)carbamate (Intermediate 1-22, 300 mg, 0.525 mmol) in Step A, the 6-step reaction procedure sequence (Steps A, B, C, F, E and G, in this order) described for Example 1 was used to prepare the title compounds. 30 (5 mg) was obtained as a white solid. 31 (4.6 mg, white solid), originated from de-bromination product at corresponding Step C, Heck coupling, and was not separated until the last step, corresponding Step G.

Compound 30: MS (ESI): 673 m/z [M+H]⁺, retention time: 1.92 minutes, purity: 97% (254 nm) (LC-MS method 14). 1H NMR (400 MHz, CD₃OD) 7.43-7.41 (m, 1H), 7.36-7.33 (m, 1H), 7.31-7.03 (m, 5H), 6.77-6.73 (m, 2H), 6.56-6.53 (m, 1H), 4.07-4.03 (m, 1H), 3.28-3.13 (m, 2H), 3.11 (s, 1H), 2.97-2.79 (m, 8H), 2.62-2.50 (m, 2H), 1.69-1.51 (m, 2H), 1.38-1.15 (m, 4H), 0.91-0.83 (m, 6H) ppm.

Compound 31: MS (ESI): 601 m/z [M+H]⁺, retention time: 1.97 minutes, purity: 99% (254 nm) (LC-MS method 14). ¹H NMR (400 MHz, CD₃OD) 7.44-7.42 (m, 1H), 7.37-7.33 (m, 1H), 7.30-7.04 (m, 6H), 6.98-6.77 (m, 2H), 6.57-6.53 (m, 1H), 4.06-4.03 (m, 1H), 3.37-3.18 (m, 2H), 3.02-2.79 (m, 6H), 2.09-1.90 (m, 1H), 1.69-1.50 (m, 2H), 1.40-1.29 (m, 2H), 1.28-1.05 (m, 2H), 0.93-0.82 (m, 6H) ppm.

### Example 32. Compound 32. 3-[3-(22,28-Difluoro-9,9-dimethyl-12-oxo-24-oxa-3,11,19,30-tetrazapentacyclo[23.3.1.12,5.015,23.016,20]triaconta-1(29),2,4,15,17,20,22,25,27-nonaen-6-yl)-2-fluoro-phenyl]propanoic acid

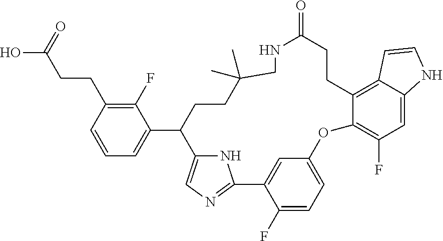 [SMILES: CC1(C)CCC(c2cccc(CCC(=O)O)c2F)c2cnc([nH]2)-c2cc(ccc2F)Oc2c(F)cc3[nH]ccc3c2CCC(=O)NC1]

Exchanging 8-bromo-6-(3-iodophenyl)-7-oxooctanenitrile (Intermediate 1-1) with 7-bromo-5-(3-bromo-2-fluorophenyl)-2,2-dimethyl-6-oxoheptanenitrile (Intermediate 1-23, 653 mg, 1.61 mmol), the reaction procedure sequence (Steps A, B, C, D, E, and G) described for Example 1 was used to prepare the title compound (9.5 mg). The corresponding Step F was not necessary because the Raney-Nickle catalyzed hydrogenation in corresponding Step D was complete. MS (ESI): 645 m/z [M+H]⁺, retention time: 1.77 minutes, purity: >99% (214 nm) (LC-MS method 7). ¹H NMR (400 MHz, CD₃OD) δ 7.43-7.41 (m, 1H), 7.30-7.20 (m, 4H), 7.09-7.05 (m, 1H), 6.97-6.91 (m, 2H), 6.79 (s, 1H), 6.57 (d, J=3.2 Hz, 1H), 4.24-4.18 (m, 1H), 3.24-3.14 (m, 2H), 3.12-3.08 (m, 1H), 2.93 (t, J=7.6 Hz, 2H), 2.56-2.21 (m, 5H), 1.94-1.56 (m, 2H), 1.4-1.00 (m, 2H), 0.69 (s, 3H), 0.59 (s, 3H) ppm.

### Example 33. Compound 33. 3-[3-(22,28-Difluoro-6,9,9,11-tetramethyl-12-oxo-24-oxa-3,11,19,30-tetrazapentacyclo[23.3.1.12,5.015,23.016,20]triaconta-1(29),2,4,15,17,20,22,25,27-nonaen-6-yl)phenyl]propanoic acid

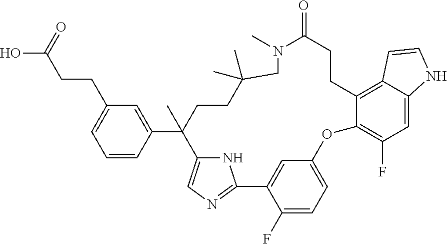 [SMILES: CN1CC(C)(C)CCC(C)(c2cccc(CCC(=O)O)c2)c2cnc([nH]2)-c2cc(ccc2F)Oc2c(F)cc3[nH]ccc3c2CCC1=O]

Exchanging 8-bromo-6-(3-iodophenyl)-7-oxooctanenitrile (Intermediate 1-1) with benzyl (7-bromo-5-(3-iodophenyl)-2,2,5-trimethyl-6-oxoheptyl)(methyl)carbamate (Intermediate 1-24, 300 mg, 0.525 mmol), the 6-step reaction procedure sequence (Steps A, B, C, F, E and G, in this order) described for Example 1 was used to prepare the title compounds (42 mg, white solid). MS (ESI): 655.3 m/z [M+H]⁺, retention time: 1.53 minutes, purity: >99% (214 nm) (LC-MS method 5).

### Example 34. Compound 34. 3-[3-(23,29-Difluoro-6,12-dimethyl-13-oxo-spiro[25-oxa-3,12,20,31-tetrazapentacyclo[24.3.1.12,5.016,24.017,21]hentriaconta-1(30),2,4,16,18,21,23,26,28-nonaene-10,1′-cyclopropane]-6-yl)phenyl]propanoic acid

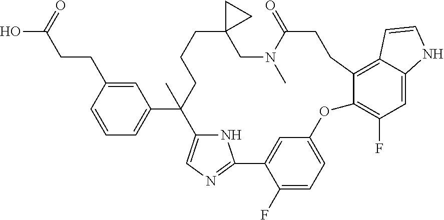 [SMILES: CN1CC2(CCCC(C)(c3cccc(CCC(=O)O)c3)c3cnc([nH]3)-c3cc(ccc3F)Oc3c(F)cc4[nH]ccc4c3CCC1=O)CC2]

### Example 35. Compound 35. 23,29-Difluoro-6,12-dimethyl-6-phenyl-spiro[25-oxa-3,12,20,31-tetrazapentacyclo[24.3.1.12,5.016,24.017,21]hentriaconta-1(30),2,4,16,18,21,23,26,28-nonaene-10,1′-cyclopropane]-13-one

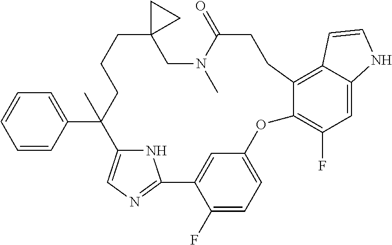 [SMILES: CN1CC2(CCCC(C)(c3ccccc3)c3cnc([nH]3)-c3cc(ccc3F)Oc3c(F)cc4[nH]ccc4c3CCC1=O)CC2]

### (E)-3-(5-(3-(5-(2-(3-Bromophenyl)-5-(1-(hydroxymethyl)cyclopropyl)pentan-2-yl)-1H-imidazol-2-yl)-4-fluorophenoxy)-6-fluoro-1H-indol-4-yl)acrylic acid

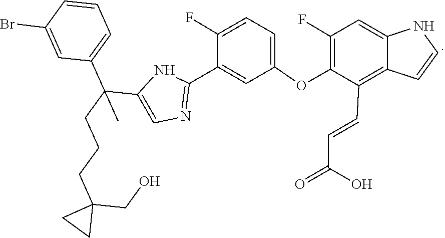 [SMILES: CC(CCCC1(CO)CC1)(c1cccc(Br)c1)c1cnc(-c2cc(Oc3c(F)cc4[nH]ccc4c3/C=C/C(=O)O)ccc2F)[nH]1]

Step A: Exchanging 7-bromo-5-(3-bromo-2-fluorophenyl)-5-methyl-6-oxoheptyl acetate (Intermediate 1-17) with (1-(6-bromo-4-(3-bromophenyl)-4-methyl-5-oxohexyl)cyclopropyl)methyl acetate (Intermediate 1-25, 0.7 g, 0.00152 mol), the reaction procedure sequence (Steps A and B) described for Example 80 was used to prepare the title compounds (0.74 g, 77% two steps) as a yellow solid. MS (ESI): 676 m/z, 678 [M+H]⁺, retention time: 1.82 minutes, purity: 82% (214 nm) (LC-MS method 7).

### (E)-3-(5-(3-(5-(2-(3-Bromophenyl)-5-(1-formylcyclopropyl)pentan-2-yl)-1H-imidazol-2-yl)-4-fluorophenoxy)-6-fluoro-1H-indol-4-yl)acrylic acid

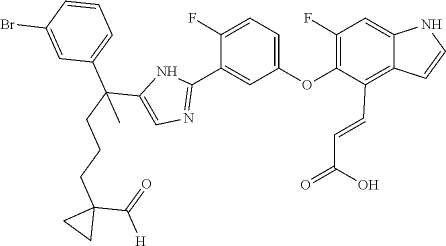 [SMILES: CC(CCCC1(C=O)CC1)(c1cccc(Br)c1)c1cnc(-c2cc(Oc3c(F)cc4[nH]ccc4c3/C=C/C(=O)O)ccc2F)[nH]1]

Step B: To a stirred solution of Step A product (0.54 g, 0.798 mmol) in dimethyl sulfoxide (20 mL) was added 2-iodoxybenzoic acid (0.671 g, 2.39 mol). The mixture was heated at 50° C. for 2 hours, then diluted with water (50 mL), and extracted with ethyl acetate (2×50 mL). The combined organic extracts were washed with brine, dried over sodium sulfate, and concentrated to give crude title compound (0.5 g, crude) as a yellow solid. MS (ESI): 674, 676 m/z [M+H]⁺, retention time: 1.83 minutes, purity: 82% (214 nm) (LC-MS method 7).

### (E)-3-(5-(3-(5-(2-(3-bromophenyl)-5-(1-((methylamino)methyl)cyclopropyl)pentan-2-yl)-1H-imidazol-2-yl)-4-fluorophenoxy)-6-fluoro-1H-indol-4-yl)acrylic acid

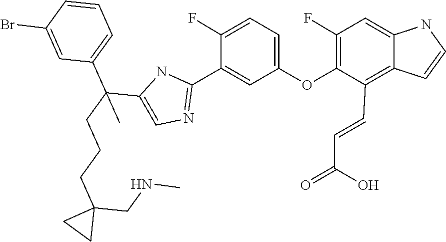 [SMILES: CNCC1(CCCC(C)(c2cccc(Br)c2)c2cnc(-c3cc(Oc4c(F)cc5[nH]ccc5c4/C=C/C(=O)O)ccc3F)[nH]2)CC1]

Step C: To a stirred solution of Step B product (0.5 g, 0.74 mol) in ethanol (anhydrous, 20 mL) was added 2 M methylamine in tetrahydrofuran (2M, 3.71 mL, 7.41 mmol). The solution was stirred for 16 hours, then treated with sodium borohydride (0.169 g, 4.45 mmol). The reaction mixture was stirred at room temperature for 1.5 hours and concentrated. The residue was purified by Prep-HPLC (0-90% acetonitrile in water with 0.1% ammonium bicarbonate as modifier) to give the title compound (0.36 g, 66% two steps) as a yellow solid. MS (ESI): 689, 691 m/z [M+H]⁺, retention time: 1.87 minutes, purity: >99% (214 nm) (LC-MS method 7).

### Compound 34: 3-[3-(23,29-Difluoro-6,12-dimethyl-13-oxo-spiro[25-oxa-3,12,20,31-tetrazapentacyclo[24.3.1.12,5.016,24.017,21]hentriaconta-1(30),2,4,16,18,21,23,26,28-nonaene-10,1′-cyclopropane]-6-yl)phenyl]propanoic acid

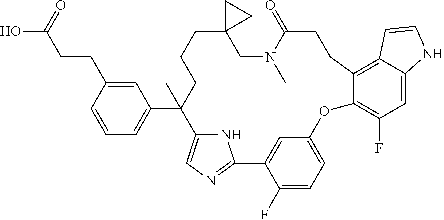 [SMILES: CN1CC2(CCCC(C)(c3cccc(CCC(=O)O)c3)c3cnc([nH]3)-c3cc(ccc3F)Oc3c(F)cc4[nH]ccc4c3CCC1=O)CC2]

Compound 35: 23,29-Difluoro-6,12-dimethyl-6-phenyl-spiro[25-oxa-3,12,20,31-tetrazapentacyclo[24.3.1.12,5.016,24.017,21]hentriaconta-1(30),2,4,16,18,21,23,26,28-nonaene-10,1′-cyclopropane]-13-one

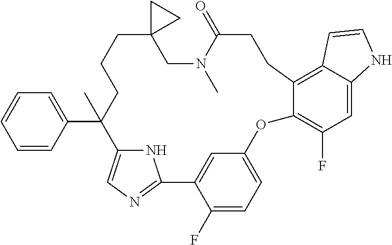 [SMILES: CN1CC2(CCCC(C)(c3ccccc3)c3cnc([nH]3)-c3cc(ccc3F)Oc3c(F)cc4[nH]ccc4c3CCC1=O)CC2]

Step D: Exchanging the Mixture of (E)-3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)acrylic acid and 3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)propanoic acid (Step D product of Example 1) with (E)-3-(5-(3-(5-(2-(3-bromophenyl)-5-(1-((methylamino)methyl)cyclopropyl)pentan-2-yl)-1H-imidazol-2-yl)-4-fluorophenoxy)-6-fluoro-1H-indol-4-yl)acrylic acid (Step C product of this Example, 0.36 g, 0.000522 mol), The reaction procedure sequence (Steps E, C, F, G, in this order) described for Example 1 was used to prepare the title compounds. 34 (130 mg) was obtained as a white solid. 35 (5 mg, white solid), originated from the de-bromination of corresponding Step C, the Heck coupling step, of Example 1, was not separated until the last step, corresponding to Step G of Example 1.

Compound 34: MS (ESI): 667.3 m/z [M+H]⁺, retention time: 1.83 minutes, purity: >99% (214 nm) (LC-MS method 7).

Compound 35: MS (ESI): 595 m/z [M+H]⁺, retention time: 1.92 minutes, purity: >99% (214 nm) (LC-MS method 7). ¹H NMR (400 MHz, CD₃OD) δ 7.17-6.99 (m, 11H), 6.37 (d, 1H), 3.13-3.09 (m, 1H), 3.96-2.90 (m, 1H), 2.91 (s, 3H), 2.78-2.62 (m, 2H), 2.52-2.49 (m, 1H), 2.31-2.29 (m, 1H), 2.20-2.16 (m, 1H), 2.04-1.97 (m, 1H), 1.81-1.72 (m, 1H), 1.60 (s, 3H), 1.36-1.26 (m, 2H), 0.85-0.78 (m, 1H), 0.49-0.39 (m, 1H), 0.35-0.25 (m, 3H).

### Example 36. Compound 36. 3-[3-(23,29-Difluoro-6-methyl-13-oxo-spiro[25-oxa-3,12,20,31-tetrazapentacyclo[24.3.1.12,5.016,24.017,21]hentriaconta-1(30),2,4,16,18,21,23,26,28-nonaene-10,1′-cyclopropane]-6-yl)phenyl]propanoic acid

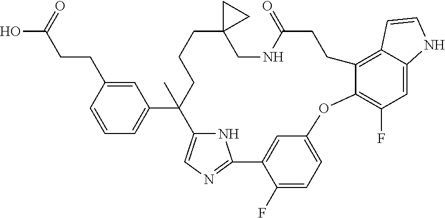 [SMILES: CC1(c2cccc(CCC(=O)O)c2)CCCC2(CC2)CNC(=O)CCc2c(c(F)cc3[nH]ccc23)Oc2ccc(F)c(c2)-c2ncc1[nH]2]

### Example 37. Compound 37: 23,29-Difluoro-6-methyl-6-phenyl-spiro[25-oxa-3,12,20,31-tetrazapentacyclo[24.3.1.12,5.016,24.017,21]hentriaconta-1(30),2,4,16,18,21,23,26,28-nonaene-10,1′-cyclopropane]-13-one

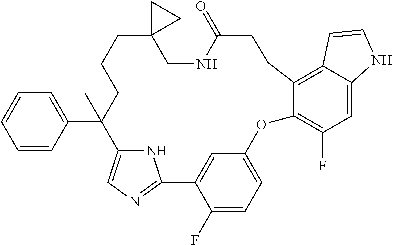 [SMILES: CC1(c2ccccc2)CCCC2(CC2)CNC(=O)CCc2c(c(F)cc3[nH]ccc23)Oc2ccc(F)c(c2)-c2ncc1[nH]2]

Exchanging 8-bromo-6-(3-iodophenyl)-7-oxooctanenitrile (Intermediate 1-1) with 6-(1-(azidomethyl)cyclopropyl)-1-bromo-3-(3-bromophenyl)-3-methylhexan-2-one (Intermediate 1-26, 0.52 g, 0.00117 mol), the reaction procedure sequence (Steps A to B of Example 1, followed by Step C of Example 11, then Steps E, C, F, G of Example 1, in this order) was used to prepare the title compounds. Compound 36 (50 mg) was obtained as a white solid. Compound 37 (5 mg), originated from the de-bromination product at the Heck coupling step, and was not separated until the last step, corresponding to Step G of Example 1.

Compound 36: MS (ESI): 653 m/z [M+H]⁺, retention time: 1.80 minutes, purity: >99% (214 nm) (LC-MS method 14). ¹H NMR (400 MHz, CD₃OD) δ 7.79-7.73 (m, 1H), 7.29-7.24 (m, 1H), 7.19-7.14 (m, 4H), 7.10 (s, 1H), 7.11-7.05 (m, 1H), 7.05-6.99 (m, 1H), 6.84-6.80 (m, 1H), 6.57-6.52 (m, 1H), 3.27-3.21 (m, 1H), 3.10-3.02 (m, 1H), 2.89-2.82 (m, 3H), 2.78-2.71 (m, 1H), 2.60-2.51 (m, 3H), 2.45-2.35 (m, 1H), 2.37-2.30 (m, 1H), 2.05-1.98 (m, 1H), 1.74-1.66 (m, 1H), 1.61 (s, 3H), 1.41-1.31 (m, 1H), 1.21-1.15 (m, 1H), 0.97-0.89 (m, 1H), 0.40-0.37 (m, 1H), 0.31-0.24 (m, 3H).

Compound 37: MS (ESI): 581 m/z [M+H]⁺, retention time: 1.88 minutes, purity: >99% (214 nm) (LC-MS method 14). ¹H NMR (400 MHz, CD₃OD) δ 7.83-7.80 (m, 1H), 7.27-7.20 (m, 5H), 7.18-7.10 (m, 3H), 7.06 (s, 1H), 6.73-6.70 (m, 1H), 6.56-6.52 (m, 1H), 3.24-3.18 (m, 1H), 3.07-3.02 (m, 1H), 2.81-2.75 (m, 2H), 2.62-2.57 (m, 1H), 2.43-2.34 (m, 2H), 2.01-1.95 (m, 1H), 1.76-1.68 (m, 1H), 1.60 (s, 3H), 1.38-1.31 (m, 1H), 1.24-1.14 (m, 1H), 0.95-0.88 (m, 1H), 0.37-0.35 (m, 1H), 0.29-0.21 (m, 3H).

### Example 38. Compound 38. 3-[3-(23,29-difluoro-10,10,12-trimethyl-13-oxo-25-oxa-5,12,20,31-tetrazapentacyclo[24.3.1.12,5.016,24.017,21]hentriaconta-1(30),2(31),3,16,18,21,23,26,28-nonaen-6-yl)phenyl]propanoic acid

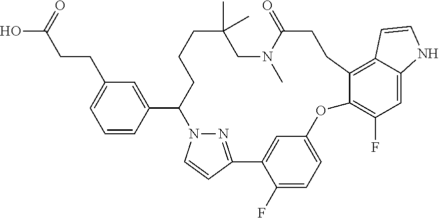 [SMILES: CN1CC(C)(C)CCCC(c2cccc(CCC(=O)O)c2)n2ccc(n2)-c2cc(ccc2F)Oc2c(F)cc3[nH]ccc3c2CCC1=O]

### Example 39. Compound 39.23,29-difluoro-10,10,12-trimethyl-6-phenyl-25-oxa-5,12,20,31-tetrazapentacyclo[24.3.1.12,5.016,24.017,21]hentriaconta-1(30),2(31),3,16,18,21,23,26,28-nonaen-13-one

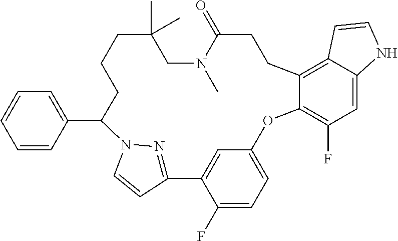 [SMILES: CN1CC(C)(C)CCCC(c2ccccc2)n2ccc(n2)-c2cc(ccc2F)Oc2c(F)cc3[nH]ccc3c2CCC1=O]

### Ethyl (E)-3-(5-(3-(1-(6-(((benzyloxy)carbonyl)(methyl)amino)-1-(3-bromophenyl)-5,5-dimethylhexyl)-1H-pyrazol-3-yl)-4-fluorophenoxy)-6-fluoro-I-tosyl-1H-indol-4-yl)acrylate

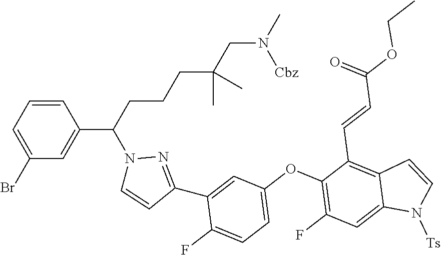 [SMILES: CCOC(=O)/C=C/c1c(Oc2ccc(F)c(-c3ccn(C(CCCC(C)(C)CN(C)C(=O)OCc4ccccc4)c4cccc(Br)c4)n3)c2)c(F)cc2c1ccn2S(=O)(=O)c1ccc(C)cc1]

Step A: To a stirred solution of benzyl (6-bromo-6-(3-bromophenyl)-2,2-dimethylhexyl)(methyl)carbamate (Intermediate 44, 0.9 g, 1.77 mmol) in acetonitrile (50 mL) was added ethyl (E)-3-(6-fluoro-5-(4-fluoro-3-(1H-pyrazol-3-yl)phenoxy)-1-tosyl-1H-indol-4-yl)acrylate (Intermediate 42, 1 g, 1.77 mmol) and cesium carbonate (1.16 g, 3.55 mmol). The mixture was stirred at 75° C. for 16 hours, cooled to room temperature and quenched with water (50 mL). The solution was extracted with ethyl acetate (3×100 mL). The combined organic phases were washed with brine (100 mL), dried over sodium sulfate, and concentrated. The residue was purified by silica gel column chromatography (eluting with ethyl acetate/petroleum ether (50:50)) to afford the title compound (0.8 g, 45%) as solid. MS (ESI): product does not ionize under analysis conditions, retention time: 2.68 minutes, purity: 66% (254 nm) (LC-MS method 9).

### Compound 38: 3-[3-(23,29-difluoro-10,10,12-trimethyl-13-oxo-25-oxa-5,12,20,31-tetrazapentacyclo[24.3.1.12,5.016,24.017,21]hentriaconta-1(30),2(31),3,16,18,21,23,26,28-nonaen-6-yl)phenyl]propanoic acid

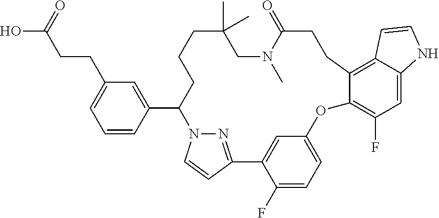 [SMILES: CN1CC(C)(C)CCCC(c2cccc(CCC(=O)O)c2)n2ccc(n2)-c2cc(ccc2F)Oc2c(F)cc3[nH]ccc3c2CCC1=O]

### Compound 39: 23,29-difluoro-10,10,12-trimethyl-6-phenyl-25-oxa-5,12,20,31-tetrazapentacyclo[24.3.1.12,5.016,24.017,21]hentriaconta-1(30),2(31),3,16,18,21,23,26,28-nonaen-13-one

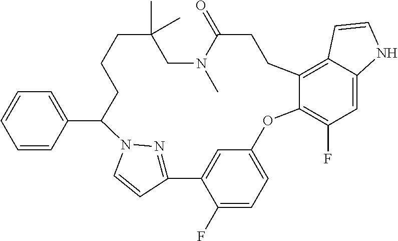 [SMILES: CN1CC(C)(C)CCCC(c2ccccc2)n2ccc(n2)-c2cc(ccc2F)Oc2c(F)cc3[nH]ccc3c2CCC1=O]

Exchanging Methyl (E)-3-(5-(3-(5-(5-cyano-1-(3-iodophenyl)pentyl)-1H-imidazol-2-yl)-4-fluorophenoxy)-6-fluoro-1H-indol-4-yl)acrylate (Step A product of Example 1) with Ethyl (E)-3-(5-(3-(1-(6-(((benzyloxy)carbonyl)(methyl)amino)-1-(3-bromophenyl)-5,5-dimethylhexyl)-1H-pyrazol-3-yl)-4-fluorophenoxy)-6-fluoro-1-tosyl-1H-indol-4-yl)acrylate (Step A product of this Example, 800 mg, 0.8 mmol), the reaction procedure sequence (Steps B, C, F, E and G, in this order) described for Example 1 was used to prepare the title compounds. 38 (20 mg) was obtained as a white solid. 39 (20 mg), the de-bromination product of corresponding Step C, Heck coupling, was not separated until the last step, Step G of Example 1.

Compound 39: MS (ESI): 583 m/z [M+H]⁺, retention time: 2.27 minutes, purity: 95% (254 nm) (LC-MS method 9). ¹H NMR (400 MHz, CD₃OD) δ 7.40-6.88 (m, 10H), 6.66-6.62 (m, 1H), 6.52-6.47 (m, 1H), 6.38-6.32 (m, 1H), 5.43-5.35 (m, 1H), 3.18-3.08 (m, 1H), 3.04-2.95 (m, 2H), 2.92-2.88 (m, 1H), 2.86-2.77 (m, 3H), 2.53-2.47 (m, 2H), 2.25-1.82 (m, 2H), 1.33-1.51 (m, 1H), 1.19-0.77 (m, 3H), 0.73-0.53 (m, 6H).

Compound 38: MS (ESI): 655 m/z [M+H]⁺, retention time: 2.03 minutes, purity: 97% (214 nm) (LC-MS method 9). ¹H NMR (400 MHz, CD₃OD) δ 7.40-6.87 (m, 9H), 6.63 (d, J=2.4 Hz, 1H), 6.49 (d, J=2.6 Hz, 1H), 6.35 (d, J=3.2 Hz, 1H), 5.36 (t, J=10.6 Hz, 1H), 3.15-2.87 (m, 3H), 2.87-2.73 (m, 5H), 2.52-2.44 (m, 4H), 1.80-2.22 (m, 2H), 1.33-1.51 (m, 1H), 1.16-0.76 (m, 4H), 0.74-0.54 (m, 6H).

### Example 40. Compound 40A, Compound 40B. Enantiomers 1 and 2 of 3-[3-(23,29-difluoro-6,10,10-trimethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoic acid

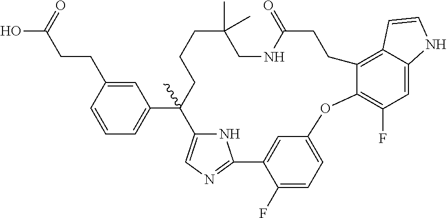 [SMILES: CC1(C)CCCC(C)(c2cccc(CCC(=O)O)c2)c2cnc([nH]2)-c2cc(ccc2F)Oc2c(F)cc3[nH]ccc3c2CCC(=O)NC1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 6.67 g, 17.3 mmol), and 8-bromo-6-(3-iodophenyl)-7-oxooctanenitrile (Intermediate 1-1) with 8-bromo-6-(3-iodophenyl)-2,2,6-trimethyl-7-oxooctanenitrile (Intermediate 1-28, 8.0 g, 17.3 mmol), the reaction procedure sequence (Steps A to E, and Step G, hydrogenation was complete in Step D so Step F was not needed) described for Example 1 was used to prepare the title compounds. The racemic ethyl ester (4 g), obtained from corresponding Step E, was subject to chiral SFC separation using SFC-80 (Thar, Waters) under the following condition: Column: SSWHELK 20*250 mm, 10 μm; Column temperature: 35° C.; Mobile phase: carbon dioxide/methanol (0.2% methanol ammonia)=30/70; Flow rate: 80 g/minute; Back pressure: 100 bar; Detection wavelength: 214 nm; Cycle time: 4.8 minutes; Sample solution: 4.3 g dissolved in 200 mL of methanol; Injection volume: 1 mL. The first eluent (1.5 g, 35%), Enantiomer 1, was subject to further ester hydrolysis to afford 40A (1.08 g, 75%); The second eluent (1.5 g, 35%), Enantiomer 2, was subject to further ester hydrolysis to afford 40A (1.31 g, 93%), as described in corresponding Step G of Example 1.

Compound 40A: MS (ESI): 655 m/z [M+H]⁺, retention time: 1.80 minutes, purity: >99% (254 nm) (LC-MS method 4).

Compound 40B: MS (ESI): 655 m/z [M+H]⁺, retention time: 1.79 minutes, purity: >99% (254 nm) (LC-MS method 4). ¹H NMR (400 MHz, CD₃OD) δ 7.87-7.83 (m, 1H), 7.32-7.27 (m, 2H), 7.24-7.15 (m, 4H), 7.12-7.06 (m, 2H), 6.92 (s, 1H), 6.59 (d, J=3.2 Hz, 1H), 3.22 (d, J=13.4 Hz, 1H), 3.15-3.07 (m, 1H), 2.89 (t, J=7.5 Hz, 3H), 2.69-2.55 (m, 4H), 2.45-2.35 (m, 1H), 2.28-2.15 (m, 2H), 1.62 (s, 3H), 1.40-1.30 (m, 2H), 1.21-1.13 (m, 1H), 0.93 (d, J=9.7 Hz, 1H), 0.83 (s, 3H), 0.67 (s, 3H).

### Example 41. Compound 41. 3-[3-(23,29-Difluoro-10,10,12-trimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

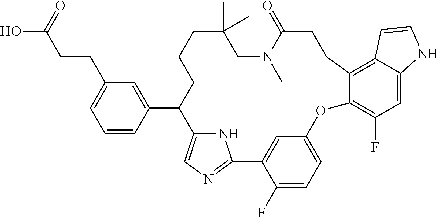 [SMILES: CN1CC(C)(C)CCCC(c2cccc(CCC(=O)O)c2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 347 mg, 0.899 mmol), and 8-bromo-6-(3-iodophenyl)-7-oxooctanenitrile (Intermediate 1-1) with benzyl (8-chloro-6-(3-iodophenyl)-2,2-dimethyl-7-oxooctyl)(methyl)carbamate (Intermediate 1-29, 500 mg, 0.899 mmol), the reaction procedure sequence (Steps A to E, and Step G, hydrogenation was complete in Step D so Step F was not needed) described for Example 1 was used to prepare the title compound (33 mg) as a white solid. MS (ESI): 655 m/z [M+H]⁺, retention time: 1.83 minutes, purity: >99% (214 nm) (LC-MS method 7). ¹H NMR (400 MHz, CD₃OD) δ 7.90-7.79 (m, 1H), 7.59-6.99 (m, 9H), 6.55-6.47 (m, 1H), 4.22-3.97 (m, 1H), 3.48-3.42 (m, 1H), 3.25-3.09 (m, 4H), 2.96-2.51 (m, 8H), 2.05-2.01 (m, 2H), 1.54-1.39 (m, 2H), 1.31 (m, 2H), 0.88 (s, 3H), 0.81 (s, 3H) ppm.

### Example 42. Compound 42A, 42A. 3-[3-(23,29-Difluoro-6,10,10,12-tetramethyl-13-oxo-9,25-dioxa-3,12,20,31-tetrazapentacyclo[24.3.1.12,5.016,24.017,21]-hentriaconta-1(30),2,4,16,18,21,23,26,28-nonaen-6-yl)phenyl]propanoic acid

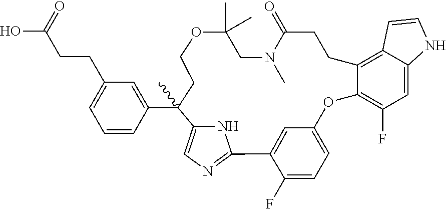 [SMILES: CN1CC(C)(C)OCCC(C)(c2cccc(CCC(=O)O)c2)c2cnc([nH]2)-c2cc(ccc2F)Oc2c(F)cc3[nH]ccc3c2CCC1=O]

### Example 43. Compound 43. 23,29-Difluoro-6,10,10,12-tetramethyl-6-phenyl-9,25-dioxa-3,12,20,31-tetrazapentacyclo[24.3.1.12,5.016,24.017,21]hentriaconta-1(30),2,4,16,18,21,23,26,28-nonaen-13-one

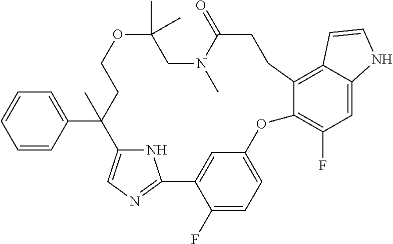 [SMILES: CN1CC(C)(C)OCCC(C)(c2ccccc2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 813 mg, 2.11 mmol), and 8-bromo-6-(3-iodophenyl)-7-oxooctanenitrile (Intermediate 1-1) with benzyl (2-((5-bromo-3-(3-iodophenyl)-3-methyl-4-oxopentyl)oxy)-2-methylpropyl)(methyl)carbamate (Intermediate 11-6, 1.3 g, 2.11 mmol), the reaction procedure sequence (Steps A, B, C, F, E and Step G, in this order) described for Example 1 was used to prepare the title compounds. The racemic ethyl ester (335 mg), obtained from corresponding Step E of Example 1, was subject to chiral SFC separation under the following conditions: Instrument: SFC-80 (Thar, Waters); Column: IG 20*250 mm, 10 μm; Column temperature: 35° C.; Mobile phase: carbon dioxide/isopropanol (0.1% diethylamine as additive)=60/40; Flow rate: 80 g/minute; Back pressure: 100 bar; Detection wavelength: 214 nm; Cycle time: 7.5 minute; Sample solution: 340 mg dissolved in 25 mL methanol; Injection volume: 2 mL. The first eluent (90 mg), Enantiomer 1, was further hydrolyzed to 42A (53.1 mg, 61%) as a white solid; The third eluent (90 mg), enantiomer 2, was further hydrolyzed to 42A (52.7 mg, 61%) as a white solid. The second eluent (70 mg), a mixture of racemic ester and 43, was also hydrolyzed to facilitate the separation. 43 (4.4 mg) was obtained as a white solid.

Compound 42A: MS (ESI): 671 m/z [M+H]⁺, retention time: 1.59 minutes, purity: 97% (214 nm) (LC-MS method 7). ¹H NMR (400 MHz, CD₃OD) δ 7.98 (s, 1H), 7.30 (d, J=3.2 Hz, 1H), 7.26-7.05 (m, 7H), 6.67-6.64 (m, 1H), 6.67-6.63 (m, 2H), 3.96-3.92 (m, 1H), 3.56-3.50 (m, 1H), 3.19-2.37 (m, 15H), 1.60 (s, 3H), 1.07 (s, 3H), 0.79 (s, 3H).

Compound 42A: MS (ESI): 671 m/z [M+H]⁺, retention time: 1.58 minutes, purity: 95% (214 nm) (LC-MS method 7). ¹H NMR (400 MHz, CD₃OD) δ 7.96 (s, 1H), 7.30 (d, J=3.2 Hz, 1H), 7.24-7.06 (m, 7H), 6.67-6.63 (m, 2H), 3.96-3.92 (m, 1H), 3.56-3.50 (m, 1H), 3.20-2.35 (m, 15H), 1.60 (s, 3H), 1.08 (s, 3H), 0.78 (s, 3H).

Compound 42B: MS (ESI): 599 m/z [M+H]⁺, retention time: 1.65 minutes, purity: 95% (214 nm) (LC-MS method 7). ¹H NMR (400 MHz, CD₃OD) δ 8.14 (s, 1H), 7.40-7.04 (m, 9H), 6.64-6.56 (m, 2H), 4.07-4.03 (m, 1H), 3.53-3.44 (m, 2H), 3.17-1.98 (m, 10H), 1.61 (s, 3H), 1.09 (s, 3H), 0.81 (s, 3H).

### Example 44. Compound 44. 3-[3-(23,29-Difluoro-6,10,10,12-tetramethyl-13-oxo-25-oxa-3,4,12,20,31-pentazapentacyclo[24.3.1.12,5.016,24.017,21]hentriaconta-1(30),2,4,16,18,21,23,26,28-nonaen-6-yl)phenyl]propanoic acid

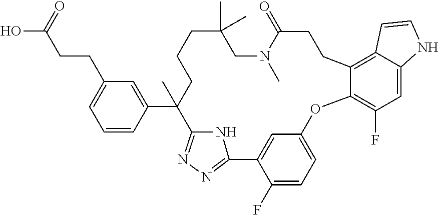 [SMILES: CN1CC(C)(C)CCCC(C)(c2cccc(CCC(=O)O)c2)c2nnc([nH]2)-c2cc(ccc2F)Oc2c(F)cc3[nH]ccc3c2CCC1=O]

### (E)-3-(6-Fluoro-5-(4-fluoro-3-(5-(7-hydroxy-2-(3-iodophenyl)-6,6-dimethylheptan-2-yl)-4H-1,2,4-triazol-3-yl)phenoxy)-1H-indol-4-yl)acrylic acid

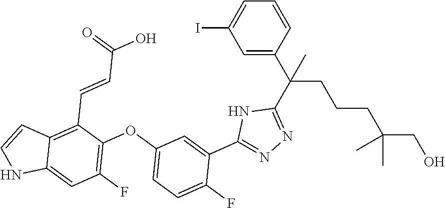 [SMILES: CC(C)(CO)CCCC(C)(c1cccc(I)c1)c1nnc(-c2cc(Oc3c(F)cc4[nH]ccc4c3/C=C/C(=O)O)ccc2F)[nH]1]

Step A: To a stirred solution of methyl (E)-3-(5-(3-carbamimidoyl-4-fluorophenoxy)-6-fluoro-1H-indol-4-yl)acrylate (Intermediate 5, 1.0 g, 2.59 mmol) in methanol (5 ml) was added 7-hydroxy-2-(3-iodophenyl)-2,6,6-trimethylheptanehydrazide (Intermediate 50, 2.10 g, 5.19 mmol) followed by sodium methoxide (0.9 mL, 5.4 M in methanol, 4.86). The mixture was stirred at 100° C. for 6 hours, then cooled to 0° C. and acidified with 1 M hydrochloric acid to pH˜4. The solution was extracted with ethyl acetate (3×30 mL). The combined organic extracts were washed with brine, dried over sodium sulfate, and concentrated. The residue was purified by automated flash chromatography (20 g silica gel column, eluting with 0-40% methanol in dichloromethane) to give the title compound (850 mg, 36%) as oil. MS (ESI): 727 m/z [M+H]⁺, retention time: 2.02 minutes, purity: 80% (214 nm) (LC-MS method 4).

### (E)-3-(3-Chloro-6-fluoro-5-(4-fluoro-3-(5-(2-(3-iodophenyl)-6,6-dimethyl-7-oxoheptan-2-yl)-4H-1,2,4-triazol-3-yl)phenoxy)-1H-indol-4-yl)acrylic acid

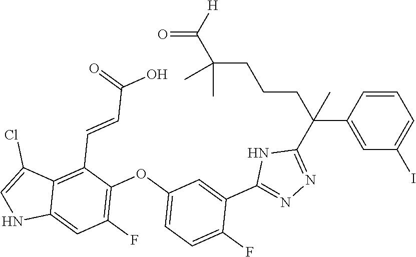 [SMILES: CC(C)(C=O)CCCC(C)(c1cccc(I)c1)c1nnc(-c2cc(Oc3c(F)cc4[nH]cc(Cl)c4c3/C=C/C(=O)O)ccc2F)[nH]1]

Step B: To a stirred solution of Step A product (850 mg, 0.8 mmol) in dichloromethane (10 mL) and N,N-dimethylformamide (5 mL) was added Dess-Martin periodinane (417 mg, 1.0 mmol). The mixture was stirred at room temperature for 2 hours, then quenched with saturated sodium bicarbonate (10 mL) and extracted with dichloromethane (2×20 mL). The combined organic phases were washed with brine, dried over sodium sulfate, and concentrated. The residue was purified by automated flash chromatography (12 g silica gel column, eluting with 0-100% ethyl acetate in petroleum) to give the title compound (399 mg, 64%) as oil. MS (ESI): 759 m/z [M+H]⁺, retention time: 2.12 minutes, purity: 50% (254 nm) (LC-MS method 4).

### (E)-3-(3-Chloro-6-fluoro-5-(4-fluoro-3-(5-(2-(3-iodophenyl)-6,6-dimethyl-7-(methylamino)heptan-2-yl)-4H-1,2,4-triazol-3-yl)phenoxy)-1H-indol-4-yl)acrylic acid

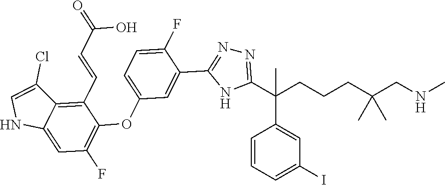 [SMILES: CNCC(C)(C)CCCC(C)(c1cccc(I)c1)c1nnc(-c2cc(Oc3c(F)cc4[nH]cc(Cl)c4c3/C=C/C(=O)O)ccc2F)[nH]1]

Step C: To a stirred solution of Step B product (399 mg, 0.526 mmol) in methanol (10 mL) was added methylamine (0.79 mL, 2 M in methanol) and three drops of acetic acid. The reaction mixture was stirred for 1 hour at room temperature, then treated with sodium cyanoborohydride (165 mg, 2.6 mmol). The mixture was stirred for 4 hours at room temperature and diluted with ethyl acetate (100 mL). The solution was washed with water, brine, dried over magnesium sulfate. The residue was purified by automated flash chromatography (12 g silica gel column, eluting with 0-30% methanol in dichloromethane) to give the title compound (158 mg, 39%) as oil. MS (ESI): 774 m/z [M+H]⁺, retention time: 1.64 minutes, purity: 50% (254 nm) (LC-MS method 4).

Compound 44: 3-[3-(23,29-Difluoro-6,10,10,12-tetramethyl-13-oxo-25-oxa-3,4,12,20,31-pentazapentacyclo[24.3.1.12,5.016,24.017,21]hentriaconta-1(30),2,4,16,18,21,23,26,28-nonaen-6-yl)phenyl]propanoic acid

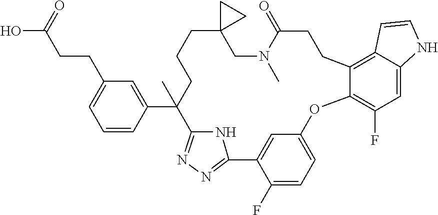 [SMILES: CN1CC2(CCCC(C)(c3cccc(CCC(=O)O)c3)c3nnc([nH]3)-c3cc(ccc3F)Oc3c(F)cc4[nH]ccc4c3CCC1=O)CC2]

Exchanging the mixture of (E)-3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)acrylic acid and 3-(5-(3-(5-(6-amino-1-(3-(3-methoxy-3-oxopropyl)phenyl)hexyl)-1H-imidazol-2-yl)-4-fluorophenoxy)-6-fluoro-1H-indol-4-yl)propanoic acid (Step D product of Example 1) with (E)-3-(3-Chloro-6-fluoro-5-(4-fluoro-3-(5-(2-(3-iodophenyl)-6,6-dimethyl-7-(methylamino)heptan-2-yl)-4H-1,2,4-triazol-3-yl)phenoxy)-1H-indol-4-yl)acrylic acid (Step C product of this example, 158 mg, 0.2 mmol), the reaction procedure sequence (Steps E, C, F, and Step G, in this order) described for Example 1 was used to prepare the title compound (3.0 mg) as a white solid. MS (ESI): 670 m/z [M+H]⁺, retention time: 1.39 minutes, purity: >99% (254 nm) (LC-MS method 4). ¹H NMR (400 MHz, CD₃OD) δ 8.02 (s, 1H), 7.45 (s, 1H), 7.36-7.03 (m, 5H), 6.97 (d, J=7.4 Hz, 1H), 6.76 (s, 1H), 6.60-6.50 (m, 1H), 3.21-2.80 (m, 9H), 2.58-2.46 (m, 2H), 2.26-1.94 (m, 2H), 1.77-1.67 (m, 3H), 1.54-0.96 (m, 6H), 0.87-0.60 (m, 6H) ppm.

### Example 45. Compound 45. 3-[3-(23,29-Difluoro-6,9,9-trimethyl-12-oxo-11,25-dioxa-3,13,20,31-tetrazapentacyclo[24.3.1.12,5.016,24.017,21]hentriaconta-1(30),2,4,16(24),17(21),18,22,26,28-nonaen-6-yl)phenyl]propanoic acid

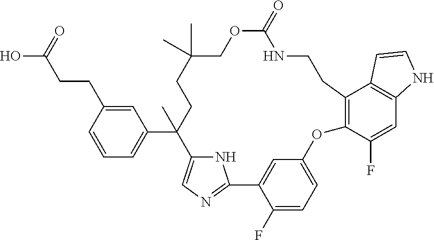 [SMILES: CC1(C)CCC(C)(c2cccc(CCC(=O)O)c2)c2cnc([nH]2)-c2cc(ccc2F)Oc2c(F)cc3[nH]ccc3c2CCNC(=O)OC1]

Exchanging ethyl (E)-3-(5-(3-carbamimidoyl-4-fluorophenoxy)-6-fluoro-1H-indol-4-yl)acrylate (Intermediate 5-2) with methyl (E)-3-(5-(3-carbamimidoyl-4-fluorophenoxy)-6-fluoro-1H-indol-4-yl)acrylate (intermediate 5, 957 mg, 2.58 mmol), and 7-bromo-5-(3-bromo-2-fluorophenyl)-5-methyl-6-oxoheptyl acetate (Intermediate 1-17) with 7-bromo-5-(3-iodophenyl)-2,2,5-trimethyl-6-oxoheptyl acetate (Intermediate 1-27, 1.16 g, 2.34 mmol), the reaction procedure sequence (Steps A to G) described for Example 80 was used to prepare the title compound (30 mg). MS (ESI): 657 m/z [M+H]⁺, retention time: 1.42 minutes, purity: >99% (214 nm) (LC-MS method 17). ¹H NMR (400 MHz, CD₃OD) δ 7.49 (s, 1H), 7.15-7.12 (m, 1H), 7.11-6.98 (m, 4H), 6.97-6.90 (m, 4H), 6.41 (s, 1H), 3.72-3.66 (m, 1H), 3.39-3.34 (m, 1H), 3.14-3.20 (m, 2H), 2.97-2.93 (m, 2H), 2.74 (t, J=7.5 Hz, 2H), 2.42 (t, J=7.6 Hz, 2H), 2.15-2.11 (m, 1H), 1.95-1.90 (m, 1H), 1.42 (s, 3H), 1.07-0.88 (m, 2H), 0.78 (s, 3H), 0.70 (s, 3H) ppm.

### Example 46. Compound 46A and Compound 46B. Enantiomers 1 and 2 of 3-[3-(24,30-difluoro-6,11,11-trimethyl-14-oxo-26-oxa-3,13,21,32-tetrazapentacyclo[25.3.1.12,5.017,25.018,22]dotriaconta-1(30),2,4,17(25),18(22),19,23,27(31),28-nonaen-6-yl)phenyl]propanoic acid

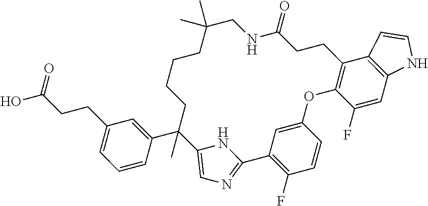 [SMILES: CC1(C)CCCCC(C)(c2cccc(CCC(=O)O)c2)c2cnc([nH]2)-c2cc(ccc2F)Oc2c(F)cc3[nH]ccc3c2CCC(=O)NC1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1.5 g, 3.87 mmol), and 8-bromo-6-(3-iodophenyl)-7-oxooctanenitrile (Intermediate 1-1) with 9-bromo-7-(3-iodophenyl)-2,2,7-trimethyl-8-oxononanenitrile (intermediate 1-32, 1.66 g, 3.5 mmol), the reaction procedure sequence (Steps A to E, and Step G. The hydrogenation in Step D was complete, so Step F was not needed) described for Example 1 was used to prepare the title compounds. The racemic ethyl ester (177 mg) from corresponding Step E, was subject to chiral SFC separation under the following condition: Instrument: SFC-80 (Thar, Waters); Column: SSWHELK 20*250 mm, 10 μm; Column temperature: 35° C.; Mobile phase: carbon dioxide/isopropanol (0.2% methanol ammonia as additive)=50/50; Flow rate: 80 g/minute; Back pressure: 100 bar; Detection wavelength: 214 nm; Cycle time: 6 minutes; Sample solution: 177 mg dissolved in 8 mL of methanol; Injection volume: 1.1 mL. The first eluent (40 mg, 100% ee), Enantiomer 1, was further hydrolyzed to 46A (15.7 mg, 41%); The second eluent (35 mg, 98% ee), Enantiomer 2, was further hydrolyzed to 46B (16.7 mg, 49%), following the conditions of Step G of Example 1.

Compound 46A: MS (ESI): 669.3 m/z [M+H]⁺, retention time: 1.40 minutes, purity: 98% (214 nm) (LC-MS method 18).

Compound 46B: MS (ESI): 669.3 m/z [M+H]⁺, retention time: 1.40 minutes, purity: 98% (214 nm) (LC-MS method 18).

### Example 47. Compound 47A and Compound 47B. Enantiomers 1 and 2 of 3-[4-(23,29-Difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoic acid

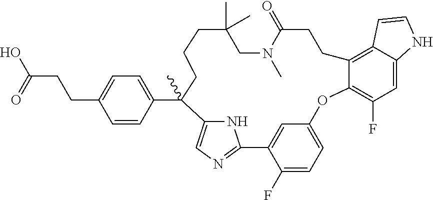 [SMILES: CN1CC(C)(C)CCCC(C)(c2ccc(CCC(=O)O)cc2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 2.2 g, 5.70 mmol), and 8-bromo-6-(3-iodophenyl)-7-oxooctanenitrile (Intermediate 1-1) with benzyl (8-bromo-6-(4-iodophenyl)-2,2,6-trimethyl-7-oxooctyl)(methyl)carbamate (intermediate 1-33, 1.66 g, 3.5 mmol), the reaction procedure sequence (Steps A to E, and Step G. The hydrogenation in Step D was complete, so Step F was not needed) described for Example 1 was used to prepare the title compounds. The racemic ethyl ester (1.0 g) from corresponding Step E, was subject to chiral SFC separation under the following condition: Instrument: SFC-80 (Thar, Waters); Column: AS 20*250 mm, 10 μm; Column temperature: 35° C.; Mobile phase: carbon dioxide/methanol (0.2% methanol ammonia as additive)=45/55; Flow rate: 80 g/minute; Back pressure: 100 bar; Detection wavelength: 214 nm; Cycle time: 7.5 minutes; Sample solution: 1.0 g dissolved in 25 mL of methanol; Injection volume: 1.0 mL. The first eluent (440 mg, 44%), Enantiomer 1, was further hydrolyzed to 47A (435 mg, 99%); The second eluent (450 mg, 45%), Enantiomer 2, was further hydrolyzed to 47B (375 mg, 89%), following the conditions of Step G of Example 1.

Compound 47A: MS (ESI): 669.3 m/z [M+H]⁺, retention time: 2.33 minutes, purity: >99% (214 nm) (LC-MS method 15).

Compound 47B: MS (ESI): 669.3 m/z [M+H]⁺, retention time: 2.33 minutes, purity: >99% (214 nm) (LC-MS method 15).

### Example 48. Compound 48A and Compound 48B. Enantiomers 1 and 2 of ethyl 3-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate

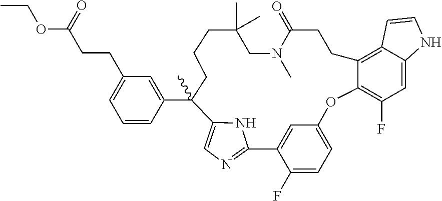 [SMILES: CCOC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1]

### Example 49. Compound 49A and Compound 49B. Enantiomers 1 and 2 of 3-[3-(23,29-Difluoro-6,10,10,12-tetramethyl-13-oxo-25-oxa-3,12,20,31-tetrazapentacyclo-[24.3.1.12,5.016,24.017,21]-hentriaconta-1(30),2,4,16,18,21,23,26,28-nonaen-6-yl)phenyl]propanoic acid

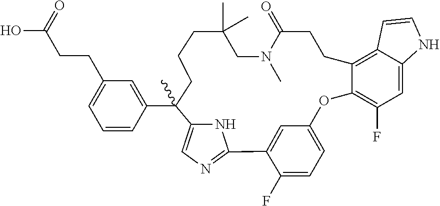 [SMILES: CN1CC(C)(C)CCCC(C)(c2cccc(CCC(=O)O)c2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 19.9 g, 51.8 mmol), and 8-bromo-6-(3-iodophenyl)-7-oxooctanenitrile (Intermediate 1-1) with benzyl (8-bromo-6-(3-iodophenyl)-2,2,6-trimethyl-7-oxooctyl)(methyl)carbamate (Intermediate 1-34, 33 g, 53.7 mmol), the reaction procedure sequence (Steps A, B, C, F, E, in this order) described for Example 1 was used to prepare the title compounds. The racemic ethyl ester (Compound 48, 15 g) from corresponding Step E of Example 1 was subject to chiral HPLC separation (corresponding analytical method: Column: Chiralpak IA, 0.46×15 cm; mobile phase: hexane/dichloromethane/ethanol=70/25/5 (V/V/V); Flow rate: 1.0 mL/minute; wavelength: 254 nm; Column temperature: 35° C.). The first eluent (6.8 g, 29%), Enantiomer 1, was designated as 48A. The second eluent (6.5 g, 28%), Enantiomer 2, was designated as 48B. Both Compound 48A (500 mg) and 48B (500 mg) were further hydrolyzed to corresponding 49A (450 mg) and 49B (460 mg), following conditions described in Step G of Example 1.

Compound 48A: MS (ESI): 697 m/z [M+H]⁺, retention time: 2.58 minutes, purity: >99% (214 nm) (LC-MS method 5). ¹H NMR (400 MHz, CD₃OD) δ 8.01 (s, 1H), 7.49 (s, 1H), 7.29-6.96 (m, 8H), 6.56-6.48 (m, 1H), 4.09-4.01 (m, 2H), 3.65-3.60 (m, 1H), 3.15-2.81 (m, 9H), 2.61-2.08 (m, 5H), 1.58-1.41 (m, 5H), 1.21-0.93 (m, 5H), 0.78-0.54 (m, 6H) ppm

Compound 48B: MS (ESI): 697 m/z [M+H]⁺, retention time: 2.59 minutes, purity: >99% (214 nm) (LC-MS method 5). ¹H NMR (400 MHz, CD₃OD) δ 8.00 (s, 1H), 7.49 (s, 1H), 7.29-6.96 (m, 8H), 6.56-6.48 (m, 1H), 4.10-4.09 (m, 2H), 3.65-3.60 (m, 1H), 3.15-2.81 (m, 9H), 2.61-2.08 (m, 5H), 1.58-1.41 (m, 5H), 1.21-0.93 (m, 5H), 0.78-0.54 (m, 6H) ppm.

Compound 49A: MS (ESI): 669.4 m/z [M+H]⁺, retention time: 1.79 minutes, purity: 93% (214 nm) (LC-MS method 15).

Compound 49B: MS (ESI): 669.4 m/z [M+H]⁺, retention time: 1.79 minutes, purity: 93% (214 nm) (LC-MS method 15).

### Example 50. Compound 50: 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanamide

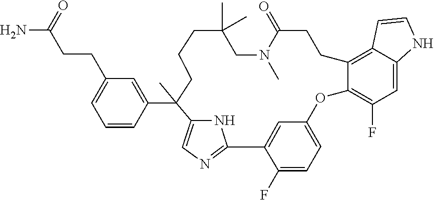 [SMILES: CN1CC(C)(C)CCCC(C)(c2cccc(CCC(N)=O)c2)c2cnc([nH]2)-c2cc(ccc2F)Oc2c(F)cc3[nH]ccc3c2CCC1=O]

To a stirred solution of 1:1 mixture of Example 49A and 49B (0.4 g, 7.48 mmol) in tetrahydrofuran (10 mL) was added 1-[bis(dimethylamino)methylene]-1H-1,2,3-triazolo[4,5-b]pyridinium 3-oxide hexafluorophosphate (HATU) (0.426 g, 1.12 mmol), ammonium chloride (1.19 g, 22.44 mmol), and triethylamine (1.13 g, 11.2 mmol). The mixture was stirred at room temperature for 16 hours and filtered. The filtrate was concentrated. The residue was purified by prep-TLC (dichloromethane/methanol=30/1) to give the title compound (0.43 g, 86%) as a white solid. MS (ESI): 668.3 m/z [M+H]⁺, retention time: 2.26 minutes, purity: 94% (214 nm) (LC-MS method 15).

### Example 51. Compound 51: 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanenitrile

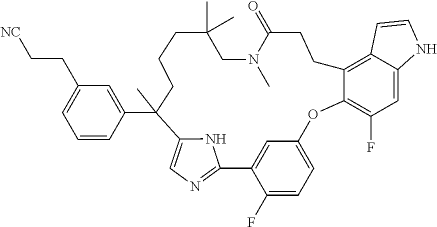 [SMILES: CN1CC(C)(C)CCCC(C)(c2cccc(CCC#N)c2)c2cnc([nH]2)-c2cc(ccc2F)Oc2c(F)cc3[nH]ccc3c2CCC1=O]

To a stirred and cooled (0° C.) solution of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanamide (Example 50, 0.43 g, 0.644 mmol) in pyridine (8 mL) was slowly added phosphoryl chloride (1.97 g, 12.9 mmol). The mixture was stirred at 0° C. for 1 hour, then neutralized to pH 5 with 2 M hydrochloric acid. The formed yellow solid was collected by filtration and dried to give the title compound (0.37 g, 88%). MS (ESI): 650.4 m/z [M+H]⁺, retention time: 1.80 minutes, purity: 80% (214 nm) (LC-MS method 7).

### Example 52. Compound 52.23,29-Difluoro-6,10,10,12-tetramethyl-6-[3-[2-(1H-tetrazol-5-yl)ethyl]phenyl]-25-oxa-3,12,20,31-tetrazapentacyclo[24.3.1.12,5.016,24.017,21]-hentriaconta-1(30),2,4,16,18,21,23,26,28-nonaen-13-one

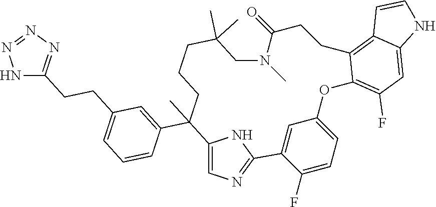 [SMILES: CN1CC(C)(C)CCCC(C)(c2cccc(CCc3nnn[nH]3)c2)c2cnc([nH]2)-c2cc(ccc2F)Oc2c(F)cc3[nH]ccc3c2CCC1=O]

To a stirred solution of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanenitrile (Example 5251, 1.89 g, 5.69 mmol) in toluene (3 mL) was added tributyltin azide (3.78 g, 11.38 mmol). The mixture was heated at 120° C. for 16 hours, cooled to room temperature, and diluted with petroleum ether (30 mL).

The formed solid was collected by filtration and purified by silica gel column chromatography (dichloromethane/methanol=10/1) to give the title compound (204 mg, 52%) as a yellow solid. MS (ESI): 693.4 m/z [M+H]⁺, retention time: 2.46 minutes, purity: >99% (214 nm) (LC-MS method 15). ¹H NMR (400 MHz, CD₃OD): δ 7.88-7.86 (m, 1H), 7.39-6.93 (m, 7H), 6.70-6.68 (m, 1H), 6.56 (d, J=2.8 Hz, 1H), 6.47 (d, J=3.2 Hz, 1H), 3.55 (d, J=13.2 Hz, 1H), 3.19-2.65 (m, 12H), 2.44-1.85 (m, 2H), 1.58-1.48 (m, 3H), 1.44-0.92 (m, 4H), 0.79-0.58 (m, 6H) ppm

### Example 53. Compound 53A and Compound 53B. Enantiomers 1 and 2 of 3-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]-N-methyl-propanamide

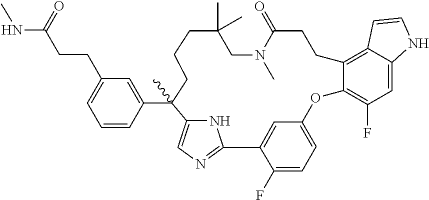 [SMILES: CNC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1]

To a stirred solution of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid (1:1 mixture of Example 49A and 49B, 250 mg, 0.374 mmol) in tetrahydrofuran (10 mL) was added methylamine hydrochloride (252 mg, 3.74 mmol), 1-[bis(dimethylamino)methylene]-1h-1,2,3-triazolo[4,5-b]pyridinium 3-oxide hexafluorophosphate (HATU, 213 mg, 0.561 mmol) and triethylamine (567 mg, 0.561 mmol). The mixture was stirred at room temperature for 16 hours and concentrated. The residue was purified by prep-TLC (dichloromethane/methanol=30/1) to afford the title compound as a white solid. This white solid was subject to chiral prep-HPLC separation to afford the first eluent as Compound 53A (65.4 mg, 26%, white solid) and the second eluent as Compound 53B (56.9 mg, 22%, white solid).

Compound 53A: MS (ESI): 682.4 m/z [M+H]⁺, retention time: 2.25 minutes, purity: >99% (214 nm) (LC-MS method 15). ¹H NMR (400 MHz, CD₃OD): 7.99 (s, 1H), 7.51 (s, 1H), 7.29-6.94 (m, 8H), 6.59-6.47 (m, 1H), 3.62 (d, J=13.2 Hz, 1H), 3.26-2.63 (m, 13H), 2.35-2.07 (m, 5H), 1.58-1.17 (m, 6H), 0.80-0.57 (m, 6H) ppm.

Compound 53B: MS (ESI): 682.4 m/z [M+H]⁺, retention time: 2.25 minutes, purity: >99% (214 nm) (LC-MS method 15). ¹H NMR (400 MHz, CD₃OD): 7.99 (s, 1H), 7.51 (s, 1H), 7.29-6.94 (m, 8H), 6.59-6.47 (m, 1H), 3.62 (d, J=13.2 Hz, 1H), 3.26-2.63 (m, 13H), 2.35-2.07 (m, 5H), 1.58-1.17 (m, 6H), 0.80-0.57 (m, 6H) ppm.

### Example 54. Compound 54. Enantiomer 1 of 3-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanehydroxamic acid

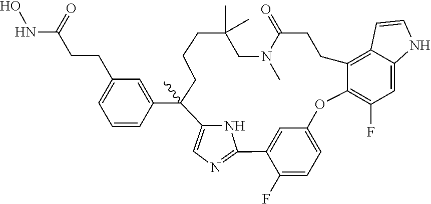 [SMILES: CN1CC(C)(C)CCCC(C)(c2cccc(CCC(=O)NO)c2)c2cnc([nH]2)-c2cc(ccc2F)Oc2c(F)cc3[nH]ccc3c2CCC1=O]

To a stirred solution of Enantiomer I of 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (Compound 48A, 200 mg, 0.299 mmol) in tetrahydrofuran (10 mL) was added hydroxylamine hydrochloride (0.208 g, 2.99 mmol), 1-[bis(dimethylamino)methylene]-1h-1,2,3-triazolo[4,5-b]pyridinium 3-oxide hexafluorophosphate (HATU, 0.170 mg, 0.499 mmol) and triethylamine (0.417 mL, 2.99 mmol). The mixture was stirred at room temperature overnight and diluted with 10 mL of tetrahydrofuran. The mixture was filtered. The filtrate was concentrated. The residue was purified by Prep-HPLC to give the title compound (43.4 mg, 21%) as a white solid. MS (ESI): 684 m/z [M+H]⁺, retention time: 1.64 minutes, purity: >99% (214 nm) (LC-MS method 5). ¹H NMR (400 MHz, CD₃OD) δ 8.25 (s, 1H), 7.88 (s, 1H), 7.29-6.98 (m, 7H), 6.74-6.71 (m, 1H), 6.56-6.47 (m, 1H), 3.55 (d, J=13 Hz, 1H), 3.18-3.11 (m, 1H), 3.03-2.64 (m, 8H), 2.61-1.86 (m, 5H), 1.59-1.51 (m, 3H), 1.46-0.95 (m, 4H), 0.79-0.60 (m, 6H) ppm.

### Example 55. Compound 55. Enantiomer 2 of 3-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanehydroxamic acid

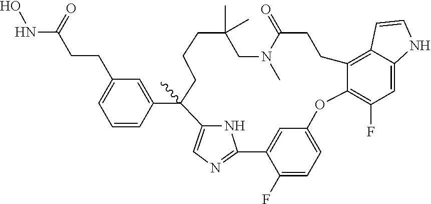 [SMILES: CN1CC(C)(C)CCCC(C)(c2cccc(CCC(=O)NO)c2)c2cnc([nH]2)-c2cc(ccc2F)Oc2c(F)cc3[nH]ccc3c2CCC1=O]

Utilizing identical conditions described for Example 54, the title compound (35.5 mg, 17%) was prepared from Compound 48B (200 mg, 0.299 mmol) as a white solid.

Compound 55: MS (ESI): 684 m/z [M+H]⁺, retention time: 1.64 minutes, purity: 98% (214 nm) (LC-MS method 5). ¹H NMR (400 MHz, CD₃OD) δ 8.25 (s, 1H), 7.88 (s, 1H), 7.29-6.98 (m, 7H), 6.74-6.71 (m, 1H), 6.56-6.47 (m, 1H), 3.55 (d, J=13 Hz, 1H), 3.18-3.11 (m, 1H), 3.03-2.64 (m, 8H), 2.61-1.86 (m, 5H), 1.59-1.51 (m, 3H), 1.46-0.95 (m, 4H), 0.80-0.60 (m, 6H) ppm.

### Example 56. Compound 56. Enantiomer 1 of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]-N-(2-methoxyethoxy)propanamide

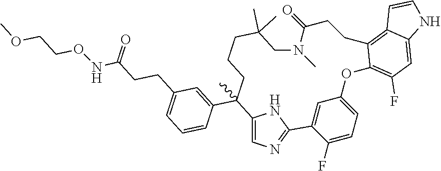 [SMILES: COCCONC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1]

Exchanging hydroxylamine hydrochloride with O-(2-methoxyethyl)hydroxylamine (Intermediate 52, 42 mg, 0.46 nmol), utilized 150 mg of Enantiomer 1 of 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (Compound 48A), the reaction condition described for Example 54 was used to prepare the title compound (118 mg, 69%) as a white solid.

Compound 56: MS (ESI): 742.4 m/z [M+H]⁺, retention time: 2.30 minutes, purity: >99% (214 nm) (LC-MS method 7).

### Example 57. Compound 57. Enantiomer 2 of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]-N-(2-methoxyethoxy)propanamide

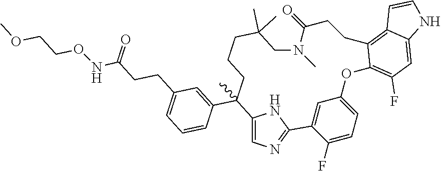 [SMILES: COCCONC(=O)CCc1cccc(C2(C)CCCC(C)(C)CN(C)C(=O)CCc3c(c(F)cc4[nH]ccc34)Oc3ccc(F)c(c3)-c3ncc2[nH]3)c1]

Exchanging hydroxylamine hydrochloride with O-(2-methoxyethyl)hydroxylamine (Intermediate 52, 34 mg, 0.37 mmol), utilized 120 mg of Enantiomer 2 of 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (Compound 48B), the reaction condition described for Example 54 was used to prepare the title compound (96 mg, 70%) as a white solid.

Compound 57: MS (ESI): 742.4 m/z [M+H]⁺, retention time: 2.30 minutes, purity: >99% (214 nm) (LC-MS method 7).

### Example 58. Compound 58. Enantiomer 1 of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]-N-(2-hydroxyethoxy)propanamide

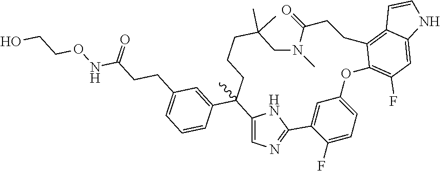 [SMILES: CN1CC(C)(C)CCCC(C)(c2cccc(CCC(=O)NOCCO)c2)c2cnc([nH]2)-c2cc(ccc2F)Oc2c(F)cc3[nH]ccc3c2CCC1=O]

Exchanging hydroxylamine hydrochloride with 2-aminooxyethanol (46 mg, 0.6 mmol), utilized 200 mg (0.3 mmol) of Enantiomer I of 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (Compound 48A), the reaction condition described for Example 54 was used to prepare the title compound (148 mg, 68%) as a white solid.

Compound 58: MS (ESI): 728.4 m/z [M+H]⁺, retention time: 2.25 minutes, purity: >99% (214 nm) (LC-MS method 7).

### Example 59. Compound 59. Enantiomer 2 of 3-[3-(23,29-difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]-N-(2-hydroxyethoxy)propanamide

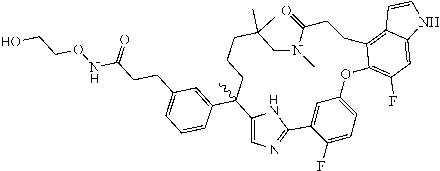 [SMILES: CN1CC(C)(C)CCCC(C)(c2cccc(CCC(=O)NOCCO)c2)c2cnc([nH]2)-c2cc(ccc2F)Oc2c(F)cc3[nH]ccc3c2CCC1=O]

Exchanging hydroxylamine hydrochloride with 2-aminooxyethanol (23 mg, 0.3 mmol), utilized 100 mg (0.15 mmol) of Enantiomer 2 of 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (Compound 48B), the reaction condition described for Example 54 was used to prepare the title compound (49 mg, 45%) as a white solid.

Compound 59: MS (ESI): 728.4 m/z [M+H]⁺, retention time: 2.24 minutes, purity: >99% (214 nm) (LC-MS method 7).

### Example 60. Compound 60A and 60B. Enantiomers 1 and 2 of 3-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]-N-methoxy-N-methyl-propanamide

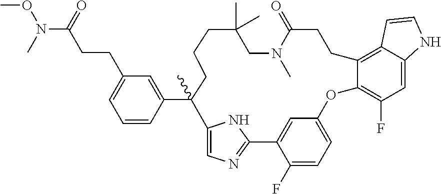 [SMILES: CON(C)C(=O)CCc1cccc(C2(C)CCCC(C)(C)CN(C)C(=O)CCc3c(c(F)cc4[nH]ccc34)Oc3ccc(F)c(c3)-c3ncc2[nH]3)c1]

Exchanging Enantiomer 1 of 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (Compound 48A) with the racemic 3-[3-(23,29-difluoro-6,10,10,12-tetramethyl-13-oxo-25-oxa-3,12,20,31-tetrazapentacyclo[24.3.1.12,5.016,24.017,21]-hentriaconta-1(30),2,4,16,18,21,23,26,28-nonaen-6-yl)phenyl]propanoic acid (equal mixture of Compounds 48A and 48B 250 mg, 0.374 mmol), and hydroxylamine hydrochloride with N,O-Dimethylhydroxylamine hydrochloride (0.365 g, 3.74 mmol), the reaction conditions described for Example 54 was used to prepare the racemic title compound mixture (210 mg, 79%) as a white solid. This racemic mixture was then subject to chiral SFC separation under the following conditions: Instrument: SFC-80 (Thar, Waters); Column: AS 20*250 mm, 10 μm; Column temperature: 35° C.; Mobile phase: carbon dioxide/methanol (0.2% methanol ammonia)=50/50; Flow rate: 80 g/minute; Back pressure: 100 bar; Detection wavelength: 214 nm; Cycle time: 7.3 minutes; Sample solution: 210 mg dissolved in 18 mL of methanol; Injection volume: 4.5 mL. The first eluent, Enantiomer 1, was designated as 60A (60.7 mg, 29%); The second eluent, enantiomer 2, was designated as 60B (64.4 mg, 31%).

Compound 60A: MS (ESI): 712 m/z [M+H]⁺, retention time: 2.38 minutes, purity: >99% (214 nm) (LC-MS method 19). ¹H NMR (400 MHz, CD₃OD): δ 8.01 (s, 1H), 7.53 (s, 1H), 7.29-6.95 (m, 8H), 6.60-6.48 (m, 1H), 3.61-3.47 (m, 3H), 3.26-2.96 (m, 6H), 2.89-2.58 (m, 10H), 2.40-1.80 (m, 3H), 1.59-1.30 (m, 6H), 0.79-0.58 (m, 6H) ppm.

Compound 60B: MS (ESI): 712 m/z [M+H]⁺, retention time: 2.38 minutes, purity: >99% (214 nm) (LC-MS method 19). ¹H NMR (400 MHz, CD₃OD): δ 8.01 (s, 1H), 7.53 (s, 1H), 7.29-6.95 (m, 8H), 6.60-6.48 (m, 1H), 3.61-3.47 (m, 3H), 3.26-2.96 (m, 6H), 2.89-2.58 (m, 10H), 2.40-1.80 (m, 3H), 1.59-1.30 (m, 6H), 0.79-0.58 (m, 6H) ppm.

### Example 61. Compound 61. 3-[3-(24,30-Difluoro-6,11,11,13-tetramethyl-14-oxo-26-oxa-3,13,21,32-tetrazapentacyclo[25.3.1.12,5.017,25.018,22]dotriaconta-1(31),2,4,17,19,22,24,27,29-nonaen-6-yl)phenyl]propanoic acid

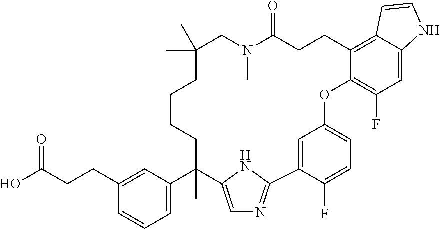 [SMILES: CN1CC(C)(C)CCCCC(C)(c2cccc(CCC(=O)O)c2)c2cnc([nH]2)-c2cc(ccc2F)Oc2c(F)cc3[nH]ccc3c2CCC1=O]

### Example 62. Compound 62. 24,30-Difluoro-6,11,11,13-tetramethyl-6-phenyl-26-oxa-3,13,21,32-tetrazapentacyclo[25.3.1.12,5.017,25.018,22]dotriaconta-1(31),2,4,17,19,22,24,27,29-nonaen-14-one

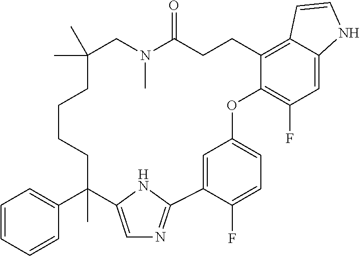 [SMILES: CN1CC(C)(C)CCCCC(C)(c2ccccc2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1.6 g, 4.15 mmol), and 8-bromo-6-(3-iodophenyl)-7-oxooctanenitrile (Intermediate 1-1) with benzyl (9-bromo-7-(3-bromophenyl)-2,2,7-trimethyl-8-oxononyl)(methyl)carbamate (Intermediate 1-36, 2.41 g, 4.15 mmol), the reaction procedure sequence (Steps A, B, C, F, E and Step G, in this order) described for Example 1 was used to prepare the title compounds. 61 (22 mg) was obtained as a white solid. 62 (22 mg), originated from de-bromination product of corresponding Step C, Heck coupling, was not separated until the last step, corresponding Step G of Example 1.

Compound 61: MS (ESI): 683.3 m/z [M+H]⁺, retention time: 1.79 minutes, purity: >99% (214 nm) (LC-MS method 5). ¹H NMR (400 MHz, CD₃OD) δ 8.00-7.85 (m, 1H), 7.69-7.50 (m, 1H), 7.43-6.98 (m, 7H), 6.80-6.70 (m, 1H), 6.56-6.51 (m, 1H), 3.29-2.46 (m, 12H), 2.40-1.72 (m, 2H), 1.62-1.03 (m, 9H), 0.90-0.79 (m, 6H), 0.64-0.60 (m, 1H) ppm.

62: MS (ESI): 611.3 m/z [M+H]⁺, retention time: 1.86 minutes, purity: >99% (214 nm) (LC-MS method 5).

### Example 63. Compound 63. 2-[4-(23,29-Difluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]acetic acid

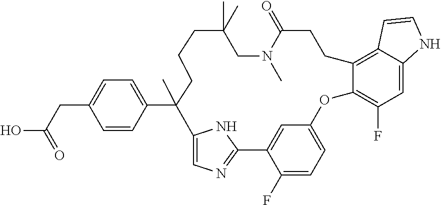 [SMILES: CN1CC(C)(C)CCCC(C)(c2ccc(CC(=O)O)cc2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with benzyl (E)-3-(5-(3-carbamimidoyl-4-fluorophenoxy)-6-fluoro-1H-indol-4-yl)acrylate (Intermediate 5-1, 186 mg, 0.32 mmol), and 8-bromo-6-(3-iodophenyl)-7-oxooctanenitrile (Intermediate 1-1) with ethyl 2-(4-(8-(((benzyloxy)carbonyl)(methyl)amino)-1-bromo-3,7,7-trimethyl-2-oxooctan-3-yl)phenyl)acetate (Intermediate 1-38, 152 mg, 0.34 mmol), the reaction procedure sequence (Steps A, F, E, F and Step G, in this order, Step F used twice because the first time hydrogenation was only partially completed on the acrylate double bond) described for Example 1 was used to prepare the title compound, 63 (24 mg) as a white solid. MS (ESI): 655 m/z [M+H]⁺, retention time: 1.76 minutes, purity: >99% (214 nm) (LC-MS method 5). ¹H NMR (400 MHz, CD₃OD) δ 7.88 (s, 1H), 7.40 (s, 1H), 7.35-7.08 (m, 6H), 6.94 (s, 1H), 6.70 (s, 1H), 6.60-6.44 (m, 1H), 3.44-3.39 (m, 2H), 3.03-2.67 (m, 7H), 2.55-1.75 (m, 4H), 1.60-1.53 (m, 3H), 1.28-0.81 (m, 4H), 0.67-0.47 (m, 6H) ppm.

### Example 64. Compound 64A, Compound 64B. Enantiomers 1 and 2 of ethyl 2-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]acetate

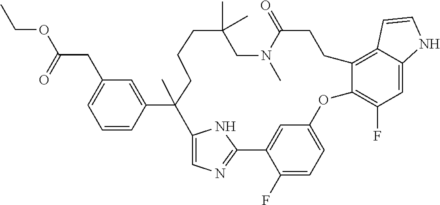 [SMILES: CCOC(=O)Cc1cccc(C2(C)CCCC(C)(C)CN(C)C(=O)CCc3c(c(F)cc4[nH]ccc34)Oc3ccc(F)c(c3)-c3ncc2[nH]3)c1]

### Example 65. Compound 65A and Compound 65B. Enantiomers 1 and 2 of 2-[3-(23,29-difluoro-6,10,10,12-tetramethyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]acetic acid

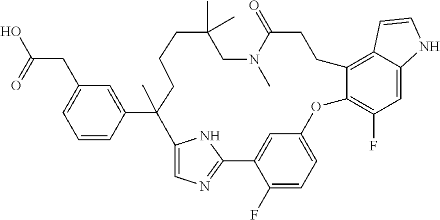 [SMILES: CN1CC(C)(C)CCCC(C)(c2cccc(CC(=O)O)c2)c2cnc([nH]2)-c2cc(ccc2F)Oc2c(F)cc3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with benzyl (E)-3-(5-(3-carbamimidoyl-4-fluorophenoxy)-6-fluoro-1H-indol-4-yl)acrylate (Intermediate 5-1, 1.85 g, 4.13 mmol,), and 8-bromo-6-(3-iodophenyl)-7-oxooctanenitrile (Intermediate 1-1) with ethyl 2-(3-(8-(((benzyloxy)carbonyl)(methyl)amino)-1-bromo-7,7-dimethyl-3-(methyl-d3)-2-oxooctan-3-yl)phenyl)acetate (Intermediate 1-49, 2.16 g, 3.75 mmol), the reaction procedure sequence (Steps A, F, E and Step G, in this order) described for Example 1 was used to prepare the title compounds. The racemic ester from corresponding Step G was subject to chiral SFC separation under the following conditions: Instrument: SFC-80 (Thar, Waters); Column: AD 20*250 mm, 10 μm; Column temperature: 35° C.; Mobile phase: carbon dioxide/ethanol (1% methanol ammonia)=65/35); Flow rate: 80 g/minute; Back pressure: 100 bar; Detection wavelength: 214 nm; Cycle time: 5.0 minutes; Sample solution: 700 mg dissolved in 15 mL of methanol; Injection volume: 1.0 mL. The first eluent, Enantiomer 1, was designated as 64A (230 mg, 33%), and further hydrolyzed to 65A (170 mg, 93%); The second eluent, Enantiomer 2, was designated as 64B (230 mg, 33%), and also further hydrolyzed to 65B (168 mg, 92%), following the conditions described in Step G of Example 1.

Compound 64A: MS (ESI): 683 m/z [M+H]⁺, retention time: 1.86 minutes, purity: >99% (214 nm) (LC-MS method 15). ¹H NMR (400 MHz, CD₃OD) δ 8.03-7.53 (m, 1H), 7.35-6.99 (m, 9H), 6.58-6.46 (m, 1H), 4.13-4.05 (m, 2H), 3.59-3.51 (m, 2H), 3.15-3.00 (m, 3H), 2.88-2.76 (m, 4H), 2.42-1.83 (m, 3H), 1.52 (s, 3H), 1.43-1.04 (m, 2H), 1.22-1.15 (m, 4H), 1.10-0.89 (m, 2H), 0.79-0.74 (m, 4H), 0.58-0.56 (m, 2H) ppm.

Compound 64B: MS (ESI): 683 m/z [M+H]⁺, retention time: 1.86 minutes, purity: 96% (214 nm) (LC-MS method 15). ¹H NMR (400 MHz, CD₃OD) δ 8.03-7.53 (m, 1H), 7.35-6.99 (m, 9H), 6.58-6.46 (m, 1H), 4.13-4.05 (m, 2H), 3.59-3.51 (m, 2H), 3.15-3.00 (m, 3H), 2.88-2.76 (m, 4H), 2.42-1.83 (m, 3H), 1.52 (s, 3H), 1.43-1.04 (m, 2H), 1.22-1.15 (m, 4H), 1.10-0.89 (m, 2H), 0.79-0.74 (m, 4H), 0.58-0.56 (m, 2H) ppm.

Compound 65A: MS (ESI): 655 m/z [M+H]⁺, retention time: 1.71 minutes, purity: >99% (214 nm) (LC-MS method 7). ¹H NMR (400 MHz, CD₃OD) δ 8.03-7.53 (m, 1H), 7.35-6.99 (m, 9H), 6.58-6.45 (m, 1H), 3.60-3.39 (m, 3H), 3.19-3.06 (m, 5H), 2.72-2.49 (m, 3H), 2.25-2.16 (m, 2H), 1.65-1.56 (m, 3H), 1.43-1.04 (m, 4H), 0.79-0.57 (m, 6H) ppm.

Compound 65B: MS (ESI): 655 m/z [M+H]⁺, retention time: 1.71 minutes, purity: >99% (214 nm) (LC-MS method 7). ¹H NMR (400 MHz, CD₃OD) δ 8.03-7.53 (m, 1H), 7.35-6.99 (m, 9H), 6.58-6.46 (m, 1H), 3.60-3.38 (m, 3H), 3.19-3.05 (m, 5H), 2.72-2.49 (m, 3H), 2.25-2.16 (m, 2H), 1.65-1.56 (m, 3H), 1.43-1.04 (m, 4H), 0.79-0.57 (m, 6H) ppm.

### Example 66. Compound 66A and Compound 66B. Enantiomers 1 and 2 of 3-[3-[(6S)-23,29-difluoro-10,10,12-trimethyl-13-oxo-6-(trideuteriomethyl)-25-oxa-3,12,20,31-tetrazapentacyclo[24.3.1.12,5.016,24.017,21]hentriaconta-1(30),2,4,16,18,21,23,26,28-nonaen-6-yl]phenyl]propanoic acid

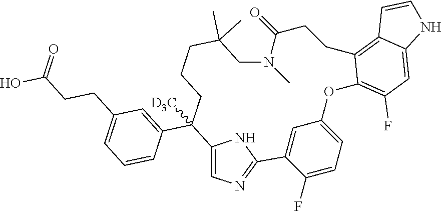 [SMILES: [2H]C([2H])([2H])C1(c2cccc(CCC(=O)O)c2)CCCC(C)(C)CN(C)C(=O)CCc2c(c(F)cc3[nH]ccc23)Oc2ccc(F)c(c2)-c2ncc1[nH]2]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1116 mg, 2.9 mmol), and 8-bromo-6-(3-iodophenyl)-7-oxooctanenitrile (Intermediate 1-1) with ethyl 2-(3-(8-(((benzyloxy)carbonyl)(methyl)amino)-1-bromo-7,7-dimethyl-3-(methyl-d3)-2-oxooctan-3-yl)phenyl)acetate (Intermediate 1-49, 1500 ng, 2.6 mmol), the reaction procedure sequence (Steps A, B, C, F, E and Step G, in this order) described for Example 1 was used to prepare the title compounds. The racemic ethyl ester (900 mg) from corresponding Step E was subject to chiral SFC separation under the following conditions: Instrument: SFC-80 (Thar, Waters); Column: AD 20*250 mm, 10 μm; Column temperature: 35° C.; Mobile phase: carbon dioxide/ethanol (1% methanol ammonia)=65/35); Flow rate: 80 g/minute; Back pressure: 100 bar; Detection wavelength: 214 nm; Cycle time: 5.0 minutes; Sample solution: 900 mg dissolved in 40 mL of methanol; Injection volume: 1.0 mL. The first eluent, Enantiomer 1 (380 mg, 42%), was further hydrolyzed to 66A (247.4 mg, 67%); The second eluent, Enantiomer 2 (390 mg, 43%), and also further hydrolyzed to 66B (245 mg, 67%), following the conditions described in Step G of Example 1.

Compound 66A: MS (ESI): 672 m/z [M+H]⁺; ¹H NMR (400 MHz, CD₃OD): δ 7.83-7.81 (m, 1H), 7.49 (s, 1H), 7.33-7.06 (m, 8H), 6.53-6.48 (m, 1H), 3.52-3.49 (m, 1H), 3.20-2.50 (m, 14H), 2.28-2.10 (m, 2H), 1.44-1.16 (m, 2H), 0.85 (s, 3H), 0.70 (s, 3H) ppm

Compound 66A: MS (ESI): 672 m/z [M+H]⁺; ¹HNMR (400 MHz, CD₃OD) δ 7.83-7.81 (m, 1H), 7.49 (s, 1H), 7.33-7.06 (m, 8H), 6.53-6.48 (m, 1H), 3.52-3.49 (m, 1H), 3.20-2.50 (m, 14H), 2.28-2.10 (m, 2H), 1.44-1.16 (m, 2H), 0.85 (s, 3H), 0.70 (s, 3H) ppm.

### Example 67. Compound 67A and 67B. Enantiomers 1 and 2 of 3-[3-(24,30-difluoro-6,11,11-trimethyl-14-oxo-9,26-dioxa-3,13,21,32-tetrazapentacyclo[25.3.1.12,5.017,25.018,22]dotriaconta-1(31),2,4,17,19,22,24,27,29-nonaen-6-yl)phenyl]propanoic acid

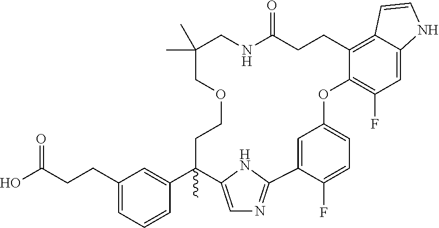 [SMILES: CC1(C)CNC(=O)CCc2c(c(F)cc3[nH]ccc23)Oc2ccc(F)c(c2)-c2ncc([nH]2)C(C)(c2cccc(CCC(=O)O)c2)CCOC1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1.76 g, 0.00450 mol), and 8-bromo-6-(3-iodophenyl)-7-oxooctanenitrile (Intermediate 1-1) with 3-((5-bromo-3-(3-bromophenyl)-3-methyl-4-oxopentyl)oxy)-2,2-dimethylpropanenitrile (Intermediate 11-7, 1.78 g, 0.00365 mol), the reaction procedure sequence (Steps A, B, C, D, E, F and Step G, in this order) described for Example 1 was used to prepare the title compounds. The racemic ethyl ester (213 mg) from corresponding Step F was subject to chiral SFC separation under the following conditions: Instrument: SFC-80 (Thar, Waters); Column: RRWHELK 20*250 mm, 10 μm; Column temperature: 35° C.; Mobile phase: carbon dioxide/isopropanol (0.2% methanol ammonia)=30/70); Flow rate: 80 g/minute; Back pressure: 100 bar; Detection wavelength: 254 nm; Cycle time: 3.0 minutes; Sample solution: 213 mg dissolved in 15 mL of methanol; Injection volume: 1.5 mL. The first eluent (71 mg, 34%), Enantiomer 1, was further hydrolyzed to 67A (52 mg, 76%); The second eluent (73 mg, 34%), Enantiomer 2, was further hydrolyzed to Compound 67B (60 mg, 89%), following the conditions described in Step G of Example 1.

Compound 67A: MS (ESI): 671 m/z [M+H]⁺, retention time: 1.68 minutes, purity: 99% (254 nm) (LC-MS method 4). ¹H NMR (400 MHz, CD₃OD): δ 7.58-7.52 (m, 1H), 7.26 (d, J=3.2 Hz, 1H), 7.23-7.11 (m, 4H), 7.08-6.99 (m, 3H), 6.98 (s, 1H), 6.56 (d, J=3.2 Hz, 1H), 3.57-3.48 (m, 1H), 3.24-3.11 (m, 3H), 3.01-2.90 (m, 3H), 2.88-2.78 (m, 3H), 2.53-2.43 (m, 5H), 2.33-2.05 (m, 1H), 1.59 (s, 3H), 0.80 (s, 3H), 0.67 (s, 3H) ppm.

Compound 67B: MS (ESI): 671 m/z [M+H]⁺, retention time: 1.68 minutes, purity: >99% (254 nm) (LC-MS method 4).

### Example 68. Compound 68. 3-[3-(21,27-Difluoro-6,11-dimethyl-12-oxo-23-oxa-3,11,18,29-tetrazapentacyclo[22.3.1.12,5.014,22.015,19]nonacosa-1(28),2,4,14,16,19,21,24,26-nonaen-6-yl)phenyl]propanoic acid

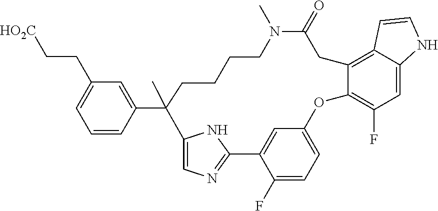 [SMILES: CN1CCCCC(C)(c2cccc(CCC(=O)O)c2)c2cnc([nH]2)-c2cc(ccc2F)Oc2c(F)cc3[nH]ccc3c2CC1=O]

### 2-(5-(3-(5-(2-(3-(3-Ethoxy-3-oxopropyl)phenyl)-6-(methylamino)hexan-2-yl)-1H-imidazol-2-yl)-4-fluorophenoxy)-6-fluoro-1H-indol-4-yl)acetic acid

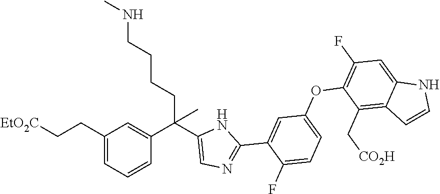 [SMILES: CCOC(=O)CCc1cccc(C(C)(CCCCNC)c2cnc(-c3cc(Oc4c(F)cc5[nH]ccc5c4CC(=O)O)ccc3F)[nH]2)c1]

Step A: Exchanging methyl (E)-3-(5-(3-carbamimidoyl-4-fluorophenoxy)-6-fluoro-1H-indol-4-yl)acrylate (Intermediate 5) with methyl 2-(5-(3-carbamimidoyl-4-fluorophenoxy)-6-fluoro-1H-indol-4-yl)acetate (Intermediate 6-1, 283 mg, 0.78 mmol), and 8-bromo-6-(3-iodophenyl)-7-oxooctanenitrile (Intermediate 1-1) with benzyl (7-bromo-5-(3-iodophenyl)-5-methyl-6-oxoheptyl)(methyl)carbamate (Intermediate 1-41, 0.45 g, 0.78 mmol), the reaction procedure sequence (Steps A, B, C, F, in this order) described for Example 1 was used to prepare the title compound (176 mg) as a light yellow solid. MS (ESI): 659 m/z [M+H]⁺, retention time: 1.44 minutes, purity: 73% (214 nm) (LC-MS method 16).

### Ethyl 3-[3-(21,27-Difluoro-6,11-dimethyl-12-oxo-23-oxa-3,11,18,29-tetrazapentacyclo[22.3.1.12,5.014,22.015,19]nonacosa-1(28),2,4,14,16,19,21,24,26-nonaen-6-yl)phenyl]propanoate

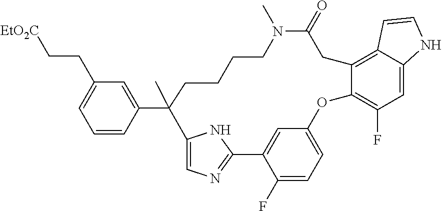 [SMILES: CCOC(=O)CCc1cccc(C2(C)CCCCN(C)C(=O)Cc3c(c(F)cc4[nH]ccc34)Oc3ccc(F)c(c3)-c3ncc2[nH]3)c1]

Step B: To a stirred solution of Step A product (150 mg, 0.23 mmol) in dichloromethane (10 mL) was added triethylamine (46 mg, 0.45 mmol) and diphenyl phosphoryl azide (0.125 g, 0.455 mmol). The mixture was stirred overnight at room temperature and concentrated. The residue was purified by flash chromatography (12 g silica gel column, eluting with 0-60% ethyl acetate in petroleum ether) to afford the title compound as an oil (50 mg, 34%). MS (ESI): 641 m/z [M+H]⁺, retention time: 1.58 minutes, purity: 84% (214 nm) (LC-MS method 9).

### Compound 68. 3-[3-(21,27-Difluoro-6,11-dimethyl-12-oxo-23-oxa-3,11,18,29-tetrazapentacyclo[22.3.1.12,5.014,22.015,19]nonacosa-1(28),2,4,14,16,19,21,24,26-nonaen-6-yl)phenyl]propanoic acid

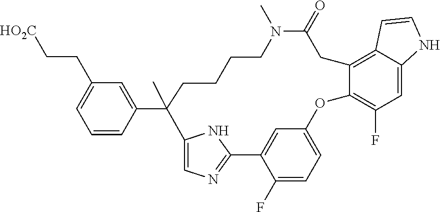 [SMILES: CN1CCCCC(C)(c2cccc(CCC(=O)O)c2)c2cnc([nH]2)-c2cc(ccc2F)Oc2c(F)cc3[nH]ccc3c2CC1=O]

Step C: To a stirred solution of Step B product (50 mg, 0.08 mmol) in methanol (3 mL) and water (1 mL) was added lithium hydroxide monohydrate (33 mg, 0.8 mmol). The mixture was stirred at room temperature for 3 hours, diluted with water (20 mL), and adjusted the pH to 5-6 with 1N hydrochloric acid. The mixture was extracted with ethyl acetate (3×20 mL). The combined organic phases were washed with water, brine, dried over sodium sulfate, and concentrated. The residue was purified by prep-TLC (dichloromethane\methanol=90\10) to give the title compound (28 mg, 59%). MS (ESI): 613 m/z [M+H]⁺, retention time: 1.37 minutes, purity: >99% (214 nm) (LC-MS method 16), ¹H NMR (400 MHz, CD₃OD) δ 7.44 (d, J=6.0 Hz, 1H), 7.35-6.95 (m, 9H), 6.27 (d, J=2.9 Hz, 1H), 4.07 (s, 2H), 3.13 (s, 3H), 2.90-2.80 (m, 4H), 2.50 (t, J=7.7 Hz, 2H), 2.46-2.41 (m, 1H), 2.13-2.05 (m, 1H), 1.57 (s, 3H), 1.42-1.37 (m, 2H), 1.21-1.06 (m, 2H) ppm.

### Example 69. Compound 69A and Compound 69B. Enantiomers 1 and 2 of 3-[3-(22,23,29-trifluoro-6-methyl-13-oxo-spiro[25-oxa-3,12,20,31-tetrazapentacyclo-[24.3.1.12,5.016,24.017,21]hentriaconta-1(30),2,4,16,18,21,23,26,28-nonaene-10,1′-cyclopropane]-6-yl)phenyl]propanoic acid

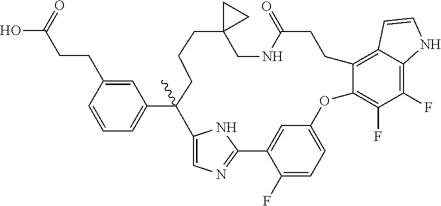 [SMILES: CC1(c2cccc(CCC(=O)O)c2)CCCC2(CC2)CNC(=O)CCc2c(c(F)c(F)c3[nH]ccc23)Oc2ccc(F)c(c2)-c2ncc1[nH]2]

Exchanging 8-bromo-6-(3-iodophenyl)-7-oxooctanenitrile (Intermediate 1-1) with 6-(1-(azidomethyl)cyclopropyl)-1-bromo-3-(3-bromophenyl)-3-methylhexan-2-one (intermediate 1-26, 1.1 g, 2.48 mmol), and methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6,7-difluoro-1H-indol-4-yl)acrylate (Intermediate 5-14, 1.1 g, 2.73 mmol), the reaction procedure sequence (Steps A, B of Example 1, followed by Step C of Example 11, then Steps E, C, F and G of Example 1, in this order) was used to prepare the title compounds. The racemic acid (100 mg), obtained from corresponding Step G of Example 1, was subjected to chiral SFC separation under the following conditions: Instrument: SFC-80 (Thar, Waters); Column: SS WHELK 20*250 mm, 10 μm; Column temperature: 35° C.; Mobile phase: carbon dioxide/methanol (0.2% methanol ammonia as additive)=40/60; Flow rate: 80 g/minute; Back pressure: 100 bar; Detection wavelength: 230 nm; Cycle time: 3 minutes; Sample solution: 100 mg dissolved in 10 mL of methanol; Injection volume: 2 mL. The first eluent, Enantiomer 1, was designated as 69A (29 mg, 100% ee). The second eluent, Enantiomer 2, was designated as 69B (30 mg, 95% ee).

Compound 69A: MS (ESI): 671 m/z [M+H]⁺, retention time: 1.59 minutes, purity: >99% (214 nm) (LC-MS method 7). ¹H NMR (400 MHz, CD₃OD) δ 7.55-7.50 (m, 1H), 7.11-7.07 (m, 1H), 6.96-6.89 (m, 3H), 6.85-6.82 (m, 2H), 6.80-6.76 (m, 1H), 6.63-6.59 (m, 1H), 6.41-6.36 (m, 1H), 3.00-2.95 (m, 1H), 2.82-2.74 (m, 1H), 2.64-2.56 (m, 3H), 2.51-2.43 (m, 1H), 2.36-2.27 (m, 3H), 2.18-2.06 (m, 2H), 1.78-1.72 (m, 1H), 1.48-1.39 (m, 1H), 1.36 (s, 3H), 1.17-1.06 (m, 1H), 0.98-0.92 (m, 1H), 0.73-0.65 (m, 1H), 0.13-0.00 (m, 4H) ppm.

Compound 69B: MS (ESI): 671 m/z [M+H]⁺, retention time: 1.59 minutes, purity: >99% (214 nm) (LC-MS method 7). ¹H NMR (400 MHz, CD₃OD) δ 7.55-7.50 (m, 1H), 7.11-7.07 (m, 1H), 6.96-6.89 (m, 3H), 6.85-6.82 (m, 2H), 6.80-6.76 (m, 1H), 6.63-6.59 (m, 1H), 6.41-6.36 (m, 1H), 3.00-2.95 (m, 1H), 2.82-2.74 (m, 1H), 2.64-2.56 (m, 3H), 2.51-2.43 (m, 1H), 2.36-2.27 (m, 3H), 2.18-2.06 (m, 2H), 1.78-1.72 (m, 1H), 1.48-1.39 (m, 1H), 1.36 (s, 3H), 1.17-1.06 (m, 1H), 0.98-0.92 (m, 1H), 0.73-0.65 (m, 1H), 0.13-0.00 (m, 4H) ppm.

### Example 70. Compound 70. 3-[3-(22,23,29-Trifluoro-6,10,10,12-tetramethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

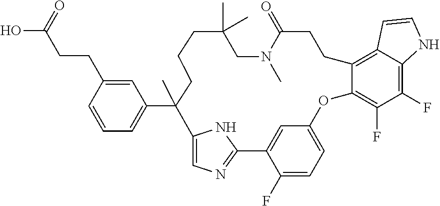 [SMILES: CN1CC(C)(C)CCCC(C)(c2cccc(CCC(=O)O)c2)c2cnc([nH]2)-c2cc(ccc2F)Oc2c(F)c(F)c3[nH]ccc3c2CCC1=O]

### Example 71. Compound 71. 22,23,29-Trifluoro-6,10,10,12-tetramethyl-6-phenyl-25-oxa-3,12,20,31-tetrazapentacyclo[24.3.1.12,5.016,24.017,21]hentriaconta-1(30),2,4,16,18,21,23,26,28-nonaen-13-one

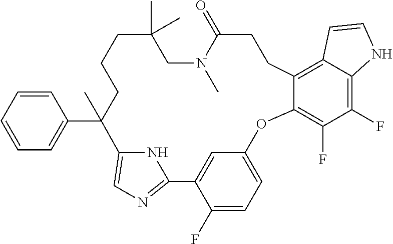 [SMILES: CN1CC(C)(C)CCCC(C)(c2ccccc2)c2cnc([nH]2)-c2cc(ccc2F)Oc2c(F)c(F)c3[nH]ccc3c2CCC1=O]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6,7-difluoro-1H-indol-4-yl)acrylate (Intermediate 5-14, 860 mg, 2.13 mmol), and 8-bromo-6-(3-iodophenyl)-7-oxooctanenitrile (Intermediate 1-1) with benzyl (8-bromo-6-(3-iodophenyl)-2,2,6-trimethyl-7-oxooctyl)(methyl)carbamate (Intermediate 1-34, 1.31 g, 2.13 mmol), the reaction procedure sequence (Steps A, B, C, F, E and G, in this order) described for Example 1 was used to prepare the title compounds. 70 (40 mg) was obtained as a white solid. 71 (9 mg, white solid), originated from the de-bromination product at corresponding Step C, Heck reaction, was not separated until corresponding Step G of Example 1.

Compound 70: MS (ESI): 687 m/z [M+H]⁺, retention time: 1.79 minutes, purity: 98% (254 nm) (LC-MS method 14). ¹H NMR (400 MHz, CD₃OD) δ 7.90-7.83 (m, 1H), 7.96-7.42 (m, 7H), 6.75-6.71 (m, 1H), 6.60-6.56 (m, 1H), 3.53-3.49 (m, 1H), 3.09-3.02 (m, 2H), 2.96-2.85 (m, 6H), 2.61-1.95 (m, 5H), 1.57-1.52 (m, 3H), 1.54-0.90 (m, 5H), 0.77-0.72 (m, 3H), 0.68-0.62 (m, 3H) ppm.

Compound 71: MS (ESI): 615 m/z [M+H]⁺, retention time: 1.86 minutes, purity: 96% (254 nm) (LC-MS method 14). ¹H NMR (400 MHz, CD₃OD) δ 8.01-6.50 (m, 11H), 3.27-2.70 (m, 6H), 2.69-1.83 (m, 4H), 1.62-1.25 (m, 6H), 1.20-0.90 (m, 2H), 0.68-0.62 (m, 6H) ppm.

### Example 72. Compound 72A, 72B. Enantiomers 1 and 2 of 3-[3-(23,29-difluoro-6,10,10,12-tetramethyl-13-oxo-25-oxa-3,12,18,20,31-pentazapentacyclo-[24.3.1.12,5.016,24.017,21]hentriaconta-1(30),2,4,16,18,21,23,26,28-nonaen-6-yl)phenyl]propanoic acid

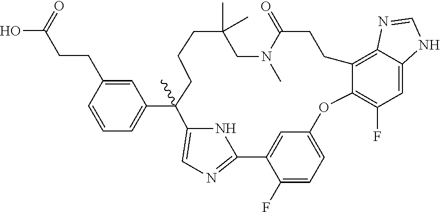 [SMILES: CN1CC(C)(C)CCCC(C)(c2cccc(CCC(=O)O)c2)c2cnc([nH]2)-c2cc(ccc2F)Oc2c(F)cc3[nH]cnc3c2CCC1=O]

### Ethyl 3-[3-(23,29-difluoro-6,10,10,12-tetramethyl-13-oxo-20-tetrahydropyran-2-yl-25-oxa-3,12,18,20,31-pentazapentacyclo[24.3.1.12,5.016,24.017,21]hentriaconta-1(30),2,4,16,18,21,23,26,28-nonaen-6-yl)phenyl]propanoate

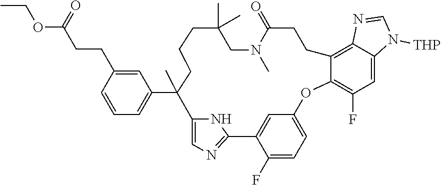

Step A: Exchanging methyl (E)-3-(5-(3-carbamimidoyl-4-fluorophenoxy)-6-fluoro-1H-indol-4-yl)acrylate (Intermediate 5) with ethyl 3-(5-(3-carbamimidoyl-4-fluorophenoxy)-6-fluoro-1-(tetrahydro-2H-pyran-2-yl)-1H-benzo[d]imidazol-4-yl)propanoate (Intermediate 6-2, 3.64 g, 5.93 mmol), and 8-bromo-6-(3-iodophenyl)-7-oxooctanenitrile (Intermediate 1-1) with benzyl (8-bromo-6-(3-iodophenyl)-2,2,6-trimethyl-7-oxooctyl)(methyl)carbamate (Intermediate 1-34, 2.80 g, 5.93 mmol), the reaction procedure sequence (Steps A, B, C, F and Step E, in this order) described for Example 1 was used to prepare the title compound (0.67 g, 14% over four steps). MS (ESI): 782 m/z [M+H]⁺, retention time: 1.90 minutes, purity: 96% (214 nm) (LC-MS method 7).

### Ethyl 3-[3-(23,29-Difluoro-6,10,10,12-tetramethyl-13-oxo-25-oxa-3,12,18,20,31-pentazapentacyclo-[24.3.1.12,5.016,24.017,21]hentriaconta-1(30),2,4,16,18,21,23,26,28-nonaen-6-yl)phenyl]propanoate

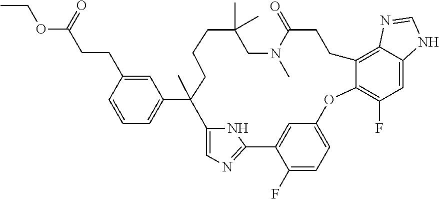 [SMILES: CCOC(=O)CCc1cccc(C2(C)CCCC(C)(C)CN(C)C(=O)CCc3c(c(F)cc4[nH]cnc34)Oc3ccc(F)c(c3)-c3ncc2[nH]3)c1]

Step B: To a solution of Step A product (630 mg, 0.806 mmol) in methanol (20 mL) was added pyridinium p-toluenesulfonate (0.81 mg, 3.22 mmol). The mixture was heated at 50° C. over the weekend, cooled to room temperature, and poured into 50 mL of saturated sodium carbonate.

The white solid was collected by filtration and air-dried to give the crude title compound (520 mg, crude). MS (ESI): 698 m/z [M+H]⁺, retention time: 1.77 minutes, purity: 94% (214 nm) (LC-MS method 5).

### Compound 72A and 72B, Enantiomers 1 and 2 of 3-[3-(23,29-Difluoro-6,10,10,12-tetramethyl-13-oxo-25-oxa-3,12,18,20,31-pentazapentacyclo-[24.3.1.12,5.016,24.017,21]hentriaconta-1(30),2,4,16,18,21,23,26,28-nonaen-6-yl)phenyl]propanoic acid

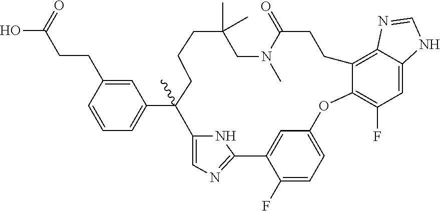 [SMILES: CN1CC(C)(C)CCCC(C)(c2cccc(CCC(=O)O)c2)c2cnc([nH]2)-c2cc(ccc2F)Oc2c(F)cc3[nH]cnc3c2CCC1=O]

Step C: The Step B product (530 mg, crude) was subjected to preparative chiral HPLC separation under the following condition: Instrument: Gilson-281; Column: AS 20*250 mm, 10 μm; Mobile Phase: Hexanes (0.1% diethylamine): ethanol (0.1% diethylamine)=85:15; Run time per injection: 15 minutes; Injection volume: 0.5 mL; Sample solution: 530 mg in 30 mL of methanol. The first eluent (150 mg, 26%), Enantiomer 1, was further hydrolyzed to 72A (127.6 mg, 88%); The second eluent (150 mg, 26%), Enantiomer 2, was further hydrolyzed to Compound 72B (87.1 mg, 70%), following the reaction conditions described in Step G of Example 1.

Compound 72A: MS (ESI): 670 m/z [M+H]⁺, retention time: 1.72 minutes, purity: >99% (214 nm) (LC-MS method 7). ¹HNMR (400 MHz, CD₃OD) δ 8.23-8.21 (m, 1H), 7.78-7.75 (m, 1H), 7.43-6.92 (m, 8H), 3.42-2.98 (m, 3H), 2.93-2.49 (m, 10H), 2.25-2.08 (m, 2H), 1.59-1.54 (m, 3H), 1.47-1.02 (m, 4H), 0.83-0.54 (m, 6H) ppm.

Compound 72B: MS (ESI): 670 m/z [M+H]⁺, retention time: 1.72 minutes, purity: >99% (214 nm) (LC-MS method 7).

### Example 73. Compound 73: 3-[3-(23,29-difluoro-6,12,12,14-tetramethyl-13-oxo-10,25-dioxa-3,14,20,31-tetrazapentacyclo[24.3.1.12,5.016,24.017,21]hentriaconta-1(30),2,4,16,18,21,23,26,28-nonaen-6-yl)phenyl]propanoic acid

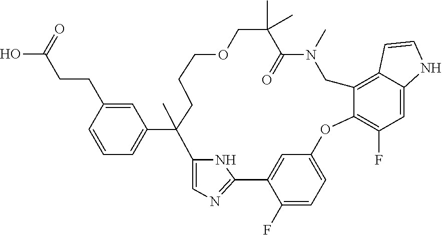 [SMILES: CN1Cc2c(c(F)cc3[nH]ccc23)Oc2ccc(F)c(c2)-c2ncc([nH]2)C(C)(c2cccc(CCC(=O)O)c2)CCCOCC(C)(C)C1=O]

### Example 74. Compound 74 23,29-difluoro-6,12,12,14-tetramethyl-6-phenyl-10,25-dioxa-3,14,20,31-tetrazapentacyclo[24.3.1.12,5.016,24.017,21]hentriaconta-1(30),2,4,16,18,21,23,26,28-nonaen-13-one

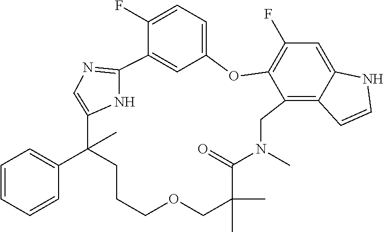 [SMILES: CN1Cc2c(c(F)cc3[nH]ccc23)Oc2ccc(F)c(c2)-c2ncc([nH]2)C(C)(c2ccccc2)CCCOCC(C)(C)C1=O]

### Methyl 3-((4-(3-bromophenyl)-4-(2-(2-fluoro-5-((6-fluoro-4-vinyl-1H-indol-5-yl)oxy)phenyl)-1H-imidazol-5-yl)pentyl)oxy)-2,2-dimethylpropanoate

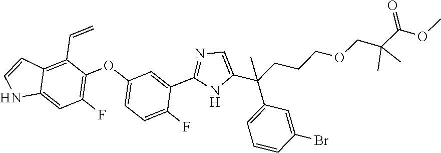 [SMILES: C=Cc1c(Oc2ccc(F)c(-c3ncc(C(C)(CCCOCC(C)(C)C(=O)OC)c4cccc(Br)c4)[nH]3)c2)c(F)cc2[nH]ccc12]

Step A: To a stirred solution of 2-fluoro-5-((6-fluoro-4-vinyl-1H-indol-5-yl)oxy)benzimidamide (Intermediate 5-3, 1.64 g, 5.23 mmol) and methyl 3-((6-bromo-4-(3-bromophenyl)-4-methyl-5-oxohexyl)oxy)-2,2-dimethylpropanoate (Intermediate 1-43, 2.5 g, 5.23 mmol) in N,N-dimethylformamide (50 mL) was added sodium bicarbonate (878 mg, 10.5 mmol). The mixture was stirred at 90° C. for 24 hours, cooled to room temperature, and diluted with water (200 mL). The solution was extracted with ethyl acetate (3×50 mL). The combined organic extracts were washed with brine, dried over sodium sulfate, and concentrated. The residue was purified by automated flash chromatography (80 g silica gel column, eluting with 0-80% ethyl acetate in petroleum ether) to give the title compound (2.5 g, 63%) as a solid. MS (ESI): 692, 694 m/z [M+H]⁺, retention time: 1.33 minutes, purity: 85% (254 nm) (LC-MS method 21).

### 3-((4-(3-Bromophenyl)-4-(2-(2-fluoro-5-((6-fluoro-4-vinyl-1H-indol-5-yl)oxy)phenyl)-1H-imidazol-5-yl)pentyl)oxy)-2,2-dimethylpropanoic acid

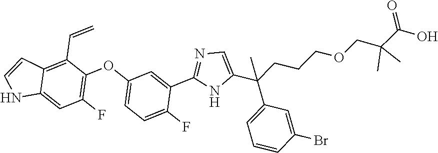 [SMILES: C=Cc1c(Oc2ccc(F)c(-c3ncc(C(C)(CCCOCC(C)(C)C(=O)O)c4cccc(Br)c4)[nH]3)c2)c(F)cc2[nH]ccc12]

Step B: To a stirred solution of Step A product (2.5 g, 3.61 mmol) in tetrahydrofuran (100 mL) and methanol (50 mL) was added lithium hydroxide monohydrate (1M in water, 50 mL, 50 mmol). The mixture was stirred at 40° C. for 16 hours and concentrated to remove tetrahydrofuran and methanol. The aqueous residue was acidified with 1N hydrochloric acid to pH˜4 and extracted with ethyl acetate (2×150 mL). The combined organic extracts were washed with water, brine, dried over sodium sulfate, and concentrated. The crude title compound (2.2 g, 90%) was obtained as a solid. MS (ESI): 678, 680 m/z [M+H]⁺, retention time: 1.27 minutes, purity: >99% (254 nm) (LC-MS method 21).

### 3-((4-(3-Bromophenyl)-4-(2-(2-fluoro-5-((6-fluoro-1-tosyl-4-vinyl-1H-indol-5-yl)oxy)phenyl)-1H-imidazol-5-yl)pentyl)oxy)-2,2-dimethylpropanoic acid

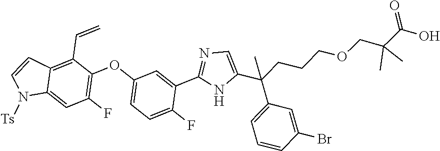 [SMILES: C=Cc1c(Oc2ccc(F)c(-c3ncc(C(C)(CCCOCC(C)(C)C(=O)O)c4cccc(Br)c4)[nH]3)c2)c(F)cc2c1ccn2S(=O)(=O)c1ccc(C)cc1]

Step C: To a stirred solution of Step B product (2.2 g, 3.24 mmol) in acetonitrile (100 mL) was added 1-(p-toluenesulfonyl)imidazole (1.44 g, 6.48 mmol) and DBU (1.25 mL, 9.73 mmol). The mixture was stirred at room temperature overnight. Another portion of 1-(p-toluenesulfonyl)imidazole (1.44 g, 6.48 mmol) was added and stirred for additional 24 hours. The solution was concentrated. The residue was dissolved in ethyl acetate (100 mL), washed with 1N hydrochloric acid, brine, dried over sodium sulfate, and concentrated. The crude product was purified by automated flash chromatography (40 g silica gel column, eluting with 0-10% methanol in dichloromethane) to give the title compound (2.8 g, 87%) as a solid. MS (ESI): 832, 834 m/z [M+H]⁺, retention time: 1.39 minutes, purity: 84% (254 nm), 63% (214 nm) (LC-MS method 21).

### 3-((4-(3-Bromophenyl)-4-(2-(5-((4-(1,2-dihydroxyethyl)-6-fluoro-1-tosyl-1H-indol-5-yl)oxy)-2-fluorophenyl)-1H-imidazol-5-yl)pentyl)oxy)-2,2-dimethylpropanoic acid

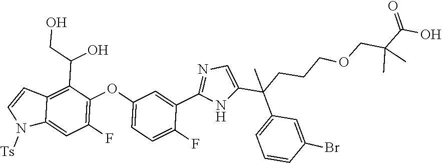 [SMILES: Cc1ccc(S(=O)(=O)n2ccc3c(C(O)CO)c(Oc4ccc(F)c(-c5ncc(C(C)(CCCOCC(C)(C)C(=O)O)c6cccc(Br)c6)[nH]5)c4)c(F)cc32)cc1]

Step D: To a stirred solution of Step C product (2.8 g, 2.82 mmol) in acetone (50 mL) and water (10 mL) was added osmium tetroxide (7.18 mL, 2 g/L) and N-methylmorpholine N-oxide (993 mg, 8.47 mmol). The mixture was stirred at room temperature for 16 hours. Another portion of osmium tetroxide (7.18 mL, 2 g/L) and N-methylmorpholine N-oxide (993 mg, 8.47 mmol) were added and stirred for an additional 4 hours. The reaction mixture was concentrated. The residue was dissolved in ethyl acetate (50 mL). The solution was washed with brine, dried over sodium sulfate, and concentrated. The crude product was purified by automated flash chromatography (40 g silica gel column, eluting with 0-10% methanol in dichloromethane) to give the title compound (2 g, 82%) as colorless oil. MS (ESI): 866, 868 m/z [M+H]⁺, retention time: 1.68 minutes, purity: 92% (254 nm), 68% (214 nm) (LC-MS method 21).

### 3-((4-(3-Bromophenyl)-4-(2-(2-fluoro-5-((6-fluoro-4-formyl-l-tosyl-1H-indol-5-yl)oxy)phenyl)-1H-imidazol-5-yl)pentyl)oxy)-2,2-dimethylpropanoic acid

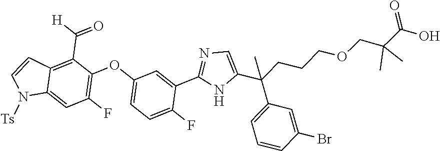 [SMILES: Cc1ccc(S(=O)(=O)n2ccc3c(C=O)c(Oc4ccc(F)c(-c5ncc(C(C)(CCCOCC(C)(C)C(=O)O)c6cccc(Br)c6)[nH]5)c4)c(F)cc32)cc1]

Step E: To a stirred solution of Step D product (2 g, 2.31 mmol) in acetone (30 mL) and water (10 mL) was added sodium periodate (1.48 g, 6.92 mmol). The mixture was stirred at room temperature overnight and concentrated to remove acetone. The residue was dissolved in ethyl acetate (100 mL). The solution was washed with brine, dried over sodium sulfate, and concentrated to give the crude title compound (1.92 g, crude) as an oil. MS (ESI): 834, 836 m/z [M+H]⁺, retention time: 1.36 minutes, purity: 97% (254 nm), 65% (214 nm) (LC-MS method 21).

### 3-((4-(3-Bromophenyl)-4-(2-(2-fluoro-5-((6-fluoro-4-((methylamino)methyl)-l-tosyl-1H-indol-5-yl)oxy)phenyl)-1H-imidazol-5-yl)pentyl)oxy)-2,2-dimethylpropanoic acid

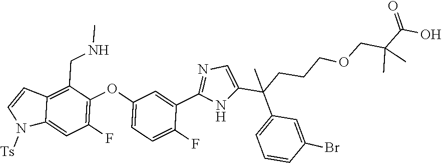 [SMILES: CNCc1c(Oc2ccc(F)c(-c3ncc(C(C)(CCCOCC(C)(C)C(=O)O)c4cccc(Br)c4)[nH]3)c2)c(F)cc2c1ccn2S(=O)(=O)c1ccc(C)cc1]

Step F: To a stirred solution of Step E product (500 mg, 0.6 mmol) in methanol (10 mL) was added methylamine hydrochloride (202 mg, 3 mmol). The mixture was stirred at room temperature overnight, then treated with triethylamine (417 μL, 3 mmol) and stirred for an additional 6 hours. To the solution was added sodium cyanoborohydride (188 mg, 3 mmol). The reaction was stirred at room temperature overnight, then quenched with water (50 mL), and extracted with ethyl acetate (3×20 mL). The combined organic extracts were washed with brine, dried over sodium sulfate, and concentrated. The crude title compound (0.5 g; 66%) was isolated as a solid. MS (ESI): 849, 851 m/z [M+H]⁺, retention time: 1.36 minutes, purity: 97% (254 nm), 65% (214 nm) (LC-MS method 21).

### 6-(3-Bromophenyl)-23,29-difluoro-6,12,12,14-tetramethyl-10,25-dioxa-3,14,20,31-tetrazapentacyclo[24.3.1.12,5.016,24.017,21]hentriaconta-1(30),2,4,16,18,21,23,26,28-nonaen-13-one

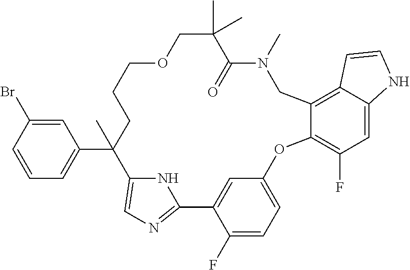 [SMILES: CN1Cc2c(c(F)cc3[nH]ccc23)Oc2ccc(F)c(c2)-c2ncc([nH]2)C(C)(c2cccc(Br)c2)CCCOCC(C)(C)C1=O]

Step G: To a stirred solution of Step F product (800 mg, 0.941 mmol) in N,N-dimethylformamide (60 mL) was added 1-[bis(dimethylamino)methylene]-1h-1,2,3-triazolo[4,5-b]pyridinium 3-oxide hexafluorophosphate (HATU) (1.43 g, 3.77 mmol) and triethylamine (525 μL, 3.77 mmol). The mixture was stirred at room temperature overnight, then stirred at 75° C. for 3 hours, and cooled to room temperature. The reaction was quenched with water (200 mL) and stirred for 0.5 hours. The precipitate was collected by filtration and the filter cake was rinsed with water (2×20 mL), and dried in vacuo.

The solid was dissolved in tetrabutylammonium fluoride (1 M in tetrahydrofuran, 15 mL). The solution was stirred at 50° C. for 3 hours, then at room temperature overnight, and concentrated. The residue was diluted with ethyl acetate (50 mL), washed with brine, dried over sodium sulfate, and concentrated. The crude product was purified by automated flash chromatography (20 g silica gel column, eluting with 0-60% ethyl acetate in petroleum ether) to give the title compound (75 mg, 11%) as a solid. MS (ESI): 677, 679 m/z [M+H]⁺, retention time: 2.01 minutes, purity: 87% (254 nm), 65% (214 nm) (LC-MS method 3).

### Compound 73: 3-[3-(23,29-Difluoro-6,12,12,14-tetramethyl-13-oxo-10,25-dioxa-3,14,20,31-tetrazapentacyclo[24.3.1.12,5.016,24.017,21]hentriaconta-1(30),2,4,16,18,21,23,26,28-nonaen-6-yl)phenyl]propanoic acid

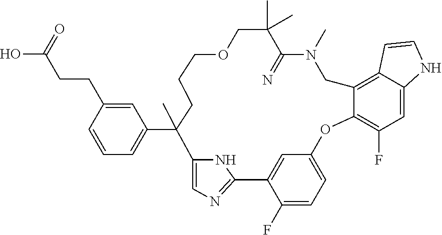 [SMILES: CN1Cc2c(c(F)cc3[nH]ccc23)Oc2ccc(F)c(c2)-c2ncc([nH]2)C(C)(c2cccc(CCC(=O)O)c2)CCCOCC(C)(C)C1=N]

### Compound 74. 23,29-Difluoro-6,12,12,14-tetramethyl-6-phenyl-10,25-dioxa-3,14,20,31-tetrazapentacyclo[24.3.1.12,5.016,24.017,21]hentriaconta-1(30),2,4,16,18,21,23,26,28-nonaen-13-one

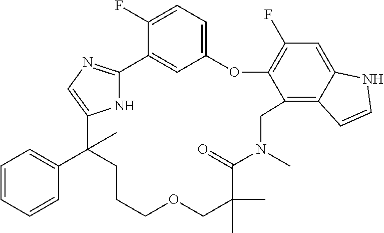 [SMILES: CN1Cc2c(c(F)cc3[nH]ccc23)Oc2ccc(F)c(c2)-c2ncc([nH]2)C(C)(c2ccccc2)CCCOCC(C)(C)C1=O]

Exchanging (E)-3-(5-(3-(5-(5-Cyano-1-(3-iodophenyl)pentyl)-1H-imidazol-2-yl)-4-fluorophenoxy)-6-fluoro-1H-indol-4-yl)acrylic acid (Step B product of Example 1) with 6-(3-bromophenyl)-23,29-difluoro-6,12,12,14-tetramethyl-10,25-dioxa-3,14,20,31-tetrazapentacyclo[24.3.1.12,5.016,24.017,21]hentriaconta-1(30),2,4,16,18,21,23,26,28-nonaen-13-one (Step G product of this example, 150 mg, 0.221 mmol), the reaction procedure sequence (Steps C, F and Step G, in this order) described for Example 1 was used to prepare the title compounds. 74A (2 mg, white solid), originated from debromination product of corresponding Step C, Heck coupling, of Example 1, was not separated until the last step, corresponding to Step G of Example 1. 73 (22.7 mg) was obtained as a white solid.

Compound 73: MS (ESI): 671 m/z [M+H]⁺, retention time: 1.79 minutes, purity: >99% (214 nm) (LC-MS method 9). ¹H NMR (400 MHz, CD₃OD) δ 7.72-7.68 (m, 1H), 7.35 (d, J=3.2 Hz, 1H), 7.29 (d, J=10.4 Hz, 1H), 7.22-7.14 (m, 3H), 7.10-7.04 (m, 3H), 6.64 (d, J=3.2 Hz, 1H), 6.58-6.55 (m, 1H), 5.26 (d, J=14.4 Hz, 1H), 4.38 (d, J=14.4 Hz, 1H), 3.71-3.64 (m, 1H), 3.57-3.45 (m, 2H), 3.11 (d, J=9.2 Hz, 1H), 2.93 (s, 3H), 2.88 (t, J=7.6 Hz, 2H), 2.71-2.61 (m, 1H), 2.55 (t, J=7.6 Hz, 2H), 2.10-1.97 (m, 1H), 1.60 (s, 3H), 1.50-1.40 (m, 2H), 1.40 (s, 3H), 1.25 (s, 3H) ppm.

Compound 74B: MS (ESI): 599 m/z [M+H]⁺, retention time: 1.86 minutes, purity: >99% (214 nm) (LC-MS method 9). ¹H NMR (400 MHz, CD₃OD) δ 7.90-7.85 (m, 1H), 7.35-7.24 (m, 6H), 7.16-7.01 (m, 3H), 6.63 (s, 1H), 6.50-6.44 (m, 1H), 5.34 (d, J=14.4 Hz, 1H), 4.28 (d, J=14.4 Hz, 1H), 3.71-3.50 (m, 2H), 3.07 (d, J=9.2 Hz, 1H), 2.92 (s, 3H), 2.76-2.69 (m, 1H), 2.23-1.94 (m, 2H), 1.62 (s, 3H), 1.55 (s, 3H), 1.33-1.31 (m, 5H) ppm.

### Example 75. Compound 75A and Compound 75B. Enantiomers 1 and 2 of 3-[3-(22,28-difluoro-3,6,9,9,11-pentamethyl-12-oxo-24-oxa-3,4,11,19,30-pentazapentacyclo[23.3.1.12,5.015,23.016,20]triaconta-1(29),2(30),4,15,17,20,22,25,27-nonaen-6-yl)phenyl]propanoic acid

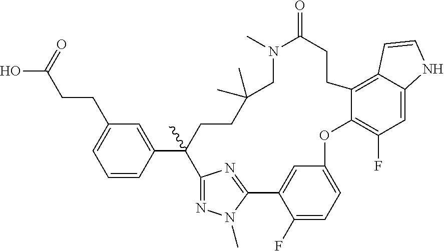 [SMILES: CN1CC(C)(C)CCC(C)(c2cccc(CCC(=O)O)c2)c2nc(n(C)n2)-c2cc(ccc2F)Oc2c(F)cc3[nH]ccc3c2CCC1=O]

### Ethyl 3-(5-(3-(3-(6-(((benzyloxy)carbonyl)(methyl)amino)-2-(3-bromophenyl)-5,5-dimethylhexan-2-yl)-1-methyl-1H-1,2,4-triazol-5-yl)-4-fluorophenoxy)-6-fluoro-1H-indol-4-yl)propanoate

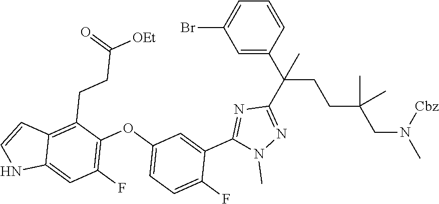 [SMILES: CCOC(=O)CCc1c(Oc2ccc(F)c(-c3nc(C(C)(CCC(C)(C)CN(C)C(=O)OCc4ccccc4)c4cccc(Br)c4)nn3C)c2)c(F)cc2[nH]ccc12]

Step A: To a stirred solution of benzyl (5-(3-bromophenyl)-2,2,5-trimethyl-6-(2-methylhydrazineyl)-6-oxohexyl)(methyl)carbamate (Intermediate 67-1, 1.50 g, 3.0 mmol) in pyridine (10 mL) was added anhydrous magnesium sulfate (0.2 g) and ethyl 3-(6-fluoro-5-(4-fluoro-3-(imino(methylthio)methyl)phenoxy)-1H-indol-4-yl)propanoate hydroiodide (Intermediate 65, 1.79 g, 3.3 mmol). The mixture was stirred at 80° C. for 16 hours, cooled to room temperature, and quenched with 1 N hydrochloric acid (10 mL). The formed precipitate was collected by filtration. The filter cake was purified by silica gel column chromatography (eluting with petroleum ether/ethyl acetate=4/1) to give the title compound (1.90 g, 74%) as a white solid. MS (ESI): 856, 858 m/z [M+H]⁺, retention time: 1.63 minutes, purity: 97% (214 nm) (LC-MS method 23).

### Compounds 75A and 75B. Enantiomers 1 and 2 of 3-[3-(22,28-difluoro-3,6,9,9,11-pentamethyl-12-oxo-24-oxa-3,4,11,19,30-pentazapentacyclo-[23.3.1.12,5.015,23.016,20]triaconta-1(29),2(30),4,15,17,20,22,25,27-nonaen-6-yl)phenyl]propanoic acid

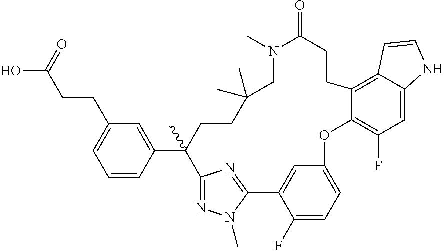 [SMILES: CN1CC(C)(C)CCC(C)(c2cccc(CCC(=O)O)c2)c2nc(n(C)n2)-c2cc(ccc2F)Oc2c(F)cc3[nH]ccc3c2CCC1=O]

Step B: Exchanging methyl (E)-3-(5-(3-(5-(5-cyano-1-(3-iodophenyl)pentyl)-1H-imidazol-2-yl)-4-fluorophenoxy)-6-fluoro-1H-indol-4-yl)acrylate (Step A product of Example 1) with ethyl 3-(5-(3-(3-(6-(((benzyloxy)carbonyl)(methyl)amino)-2-(3-bromophenyl)-5,5-dimethylhexan-2-yl)-1-methyl-1H-1,2,4-triazol-5-yl)-4-fluorophenoxy)-6-fluoro-1H-indol-4-yl)propanoate (Step A product of this example, 1.90 g, 2.2 mmol), the reaction procedure sequence (Steps B, C, F, E, and G, in this order) described for Example 1 was used to prepare the title compounds. The racemic ethyl ester (0.81 g), obtained from corresponding Step E of Example 1, was subjected to chiral SFC separation under the following conditions: Instrument: SFC-80 (Thar, Waters); Column: AD 20*250 mm, 10 μm; Column temperature: 35° C.; Mobile phase: carbon dioxide/isopropyl alcohol (0.2% methanol ammonia as additive)=65/35; Flow rate: 80 g/minute; Back pressure: 100 bar; Detection wavelength: 214 nm; Cycle time: 5.0 minutes; Sample solution: 800 mg dissolved in 22 mL of methanol; Injection volume: 0.6 mL. The first eluent (0.37 g, 43%), Enantiomer 1, was further hydrolyzed to 75A (24 mg, 74%); The second eluent (0.27 g, 34%), Enantiomer 2, was further hydrolyzed to 75B (160 mg, 62%), following the reaction conditions described in Step G of Example 1.

Compound 75A: MS (ESI): 670.3 m/z [M+H]⁺, retention time: 2.02 minutes, purity: >99% (214 nm) (LC-MS method 5).

Compound 75A: MS (ESI): 670.3 m/z [M+H]⁺, retention time: 2.02 minutes, purity: >99% (214 nm) (LC-MS method 5).

### Example 76. Compound 76A and Compound 76B. Enantiomers 1 and 2 of 3-[3-[23,29-difluoro-6,10,10-trimethyl-13-oxo-3-(trideuteriomethyl)-25-oxa-3,4,12,20,31-pentazapentacyclo[24.3.1.12,5.016,24.017,21]hentriaconta-1(30),2(31),4,16,18,21,23,26,28-nonaen-6-yl]phenyl]propanoic acid

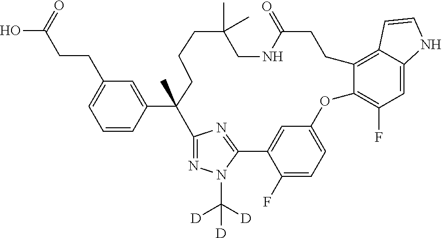 [SMILES: [2H]C([2H])([2H])n1nc2nc1-c1cc(ccc1F)Oc1c(F)cc3[nH]ccc3c1CCC(=O)NCC(C)(C)CCC[C@@]2(C)c1cccc(CCC(=O)O)c1]

Exchanging benzyl (5-(3-bromophenyl)-2,2,5-trimethyl-6-(2-methylhydrazineyl)-6-oxohexyl)(methyl)carbamate (Intermediate 67-1) with benzyl (6-(3-bromophenyl)-2,2,6-trimethyl-7-(2-(methyl-d3)hydrazineyl)-7-oxoheptyl)carbamate (Intermediate 67-2, 2 g, 3.9 mmol), the reaction procedure sequence (Step A of Example 75A and 75B, followed by Steps B, C, F, E, and G of Example 1, in this order) was used to prepare the title compounds. The racemic ethyl ester (340 mg), obtained from corresponding Step E of Example 1, was subjected to chiral SFC separation under the following condition: Instrument: SFC-80 (Thar, Waters); Column: SSWHELK 20*250 mm, 10 μm; Column temperature: 35° C.; Mobile phase: carbon dioxide/isopropanol (0.2% methanol ammonia as additive)=35/65; Flow rate: 80 g/minute; Back pressure: 100 bar; Detection wavelength: 214 nm; Cycle time: 6 minutes; Sample solution: 0.3 g dissolved in 25 mL of methanol; Injection volume: 4.5 mL. The first eluent (130 mg, 41%, white solid), Enantiomer 1, was further hydrolyzed to 76A (90 mg, 72%, white solid); The second eluent (130 mg, 41%, white solid), Enantiomer 2, was further hydrolyzed to 76B (90 mg., 72%, white solid), following conditions described in Step G of Example 1.

Compound 76A: MS (ESI): 673 m/z [M+H]⁺, retention time: 2.05 minutes, purity: 97% (214 nm) (LC-MS method 9). ¹H NMR (400 MHz, DMSO-d₆) δ 11.24 (s, 1H), 7.46 (dd, J=5.6, 3.2 Hz, 1H), 7.44-7.33 (m, 3H), 7.24 (d, J=10.8 Hz, 1H), 7.16-7.10 (m, 3H), 7.03-6.98 (m, 2H), 6.52-6.48 (m, 1H), 2.96 (t, J=7.6 Hz, 2H), 2.90-2.80 (m, 1H), 2.75 (t, J=7.6 Hz, 2H), 2.63-2.56 (m, 1H), 2.50-2.30 (m, 4H), 2.24-2.18 (m, 1H), 1.81-1.74 (m, 1H), 1.61 (s, 3H), 1.29-1.20 (m, 2H), 1.03-0.96 (m, 1H), 0.84-0.80 (m, 1H), 0.65 (s, 3H), 0.62 (s, 3H) ppm.

Compound 76B: MS (ESI): 673 m/z [M+H]⁺, retention time: 2.05 minutes, purity: 98% (214 nm) (LC-MS method 9). ¹H NMR (400 MHz, DMSO-d₆) δ 11.24 (s, 1H), 7.46 (dd, J=5.6, 3.2 Hz, 1H), 7.44-7.33 (m, 3H), 7.24 (d, J=10.8 Hz, 1H), 7.16-7.10 (m, 3H), 7.03-6.98 (m, 2H), 6.52-6.48 (m, 1H), 2.96 (t, J=7.6 Hz, 2H), 2.90-2.80 (m, 1H), 2.75 (t, J=7.6 Hz, 2H), 2.63-2.56 (m, 1H), 2.50-2.30 (m, 4H), 2.24-2.18 (m, 1H), 1.81-1.74 (m, 1H), 1.61 (s, 3H), 1.29-1.20 (m, 2H), 1.03-0.96 (m, 1H), 0.84-0.80 (m, 1H), 0.65 (s, 3H), 0.62 (s, 3H) ppm.

### Example 77. Compound 77A and Compound 77B. Enantiomers 1 and 2 of 3-[3-[23,29-difluoro-6,10,10,12-tetramethyl-13-oxo-3-(trideuteriomethyl)-25-oxa-3,4,12,20,31-pentazapentacyclo[24.3.1.12,5.016,24.017,21]-hentriaconta-1(30),2(31),4,16,18,21,23,26,28-nonaen-6-yl]phenyl]propanoic acid

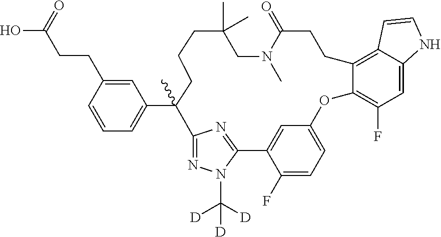 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)O)c1]

### Ethyl 3-[3-[23,29-difluoro-6,10,10,12-tetramethyl-13-oxo-3-(trideuteriomethyl)-25-oxa-3,4,12,20,31-pentazapentacyclo[24.3.1.12,5.016,24.017,21]-hentriaconta-1(30),2(31),4,16,18,21,23,26,28-nonaen-6-yl]phenyl]propanoate

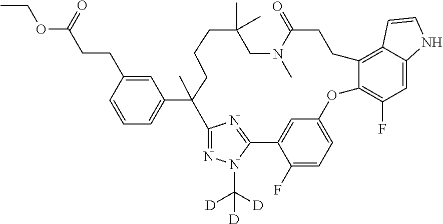 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)OCC)c1]

Step A: Exchanging benzyl (5-(3-bromophenyl)-2,2,5-trimethyl-6-(2-methylhydrazineyl)-6-oxohexyl)(methyl)carbamate (Intermediate 67-1) with benzyl (6-(3-bromophenyl)-2,2,6-trimethyl-7-(2-(methyl-d3)hydrazineyl)-7-oxoheptyl)(methyl)carbamate (Intermediate 67-3, 2.6 g, 4.99 mmol), the reaction procedure sequence (Step A of Example 75A and 75B, followed by Steps B, C, D, E, F of Example 1) was used to prepare the title compounds (1 g). MS (ESI): 715 m/z [M+H]⁺, retention time: 2.10 minutes, purity: 96% (214 nm) (LC-MS method 4).

### Compound 77A and 77B. Enantiomers 1 and 2 of 3-[3-[23,29-difluoro-6,10,10,12-tetramethyl-13-oxo-3-(trideuteriomethyl)-25-oxa-3,4,12,20,31-pentazapentacyclo[24.3.1.12,5.016,24.017,21]-hentriaconta-1(30),2(31),4,16,18,21,23,26,28-nonaen-6-yl]phenyl]propanoic acid

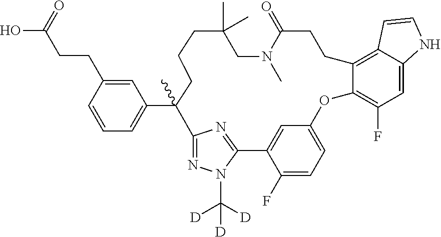 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)O)c1]

Step B: The Step A product (600 mg) was subjected to chiral prep-HPLC separation under the following condition: Instrument: Gilson-281; Column: R,R WHELK 20*250 mm, 10 μm; Mobile Phase: n-Hexane (0.1% formic acid): ethanol (0.1% formic acid)=7:3; Flow Rate: 50 mL/minute; Run time per injection: 28 minutes; Injection: 0.7 mL; Sample solution: 600 mg in 46 mL of methanol. The first eluent (200 mg, 33%), white solid), Enantiomer 1, was further hydrolyzed to 77A (141 mg, 74%, white solid); The second eluent (200 mg, 33%, white solid), Enantiomer 2, was further hydrolyzed to 77B (125 mg, 65%, white solid), following conditions described in Step G of Example 1.

Compound 77A: MS (ESI): 687 m/z [M+H]⁺, retention time: 1.90 minutes, purity: 96% (214 nm) (LC-MS method 4).

Compound 77B: MS (ESI): 687 m/z [M+H]⁺, retention time: 1.90 minutes, purity: 96% (214 nm) (LC-MS method 4).

### Example 78. Compound 78. 23,29-difluoro-6-[3-(3-hydroxypropyl)phenyl]-6,10,10,12-tetramethyl-3-(trideuteriomethyl)-25-oxa-3,4,12,20,31-pentazapentacyclo-[24.3.1.12,5.016,24.017,21]hentriaconta-1(30),2(31),4,16,18,21,23,26,28-nonaen-13-one

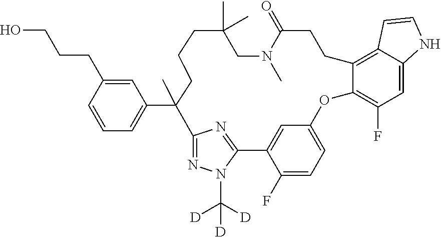 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCCO)c1]

To a stirred solution of ethyl 3-[3-[23,29-difluoro-6,10,10,12-tetramethyl-13-oxo-3-(trideuteriomethyl)-25-oxa-3,4,12,20,31-pentazapentacyclo [24.3.1.12,5.016,24.017,21]-hentriaconta-1(30),2(31),4,16,18,21,23,26,28-nonaen-6-yl]phenyl]propanoate (Step A product of Example 77A and 77B, 180 mg, 0.252 mmol) in tetrahydrofuran (5 mL) was added lithium borohydride (110 mg, 5.04 mmol). The mixture was stirred at room temperature overnight, quenched with water (40 mL), and extracted with ethyl acetate (2×30 mL). The combined organic phases were washed with brine, dried over sodium sulfate, and concentrated. The residue was purified by silica gel column chromatography (25 g silica gel column, eluting with 0-10% methanol in dichloromethane) to give the title compound (130 mg, 77%) as a solid. MS (ESI): 673 m/z [M+H]⁺, retention time: 1.93 minutes, purity: 96% (214 nm) (LC-MS method 4).

### Example 79. Compound 79A and Compound 79B. Enantiomers 1 and 2 of 3-[3-[23,29-difluoro-6,10,10,12-tetramethyl-13-oxo-3-(trideuteriomethyl)-25-oxa-3,4,12,20,31-pentazapentacyclo[24.3.1.12,5.016,24.017,21]hentriaconta-1(30),2(31),4,16,18,21,23,26,28-nonaen-6-yl]phenyl]-N-methylsulfonyl-propanamide

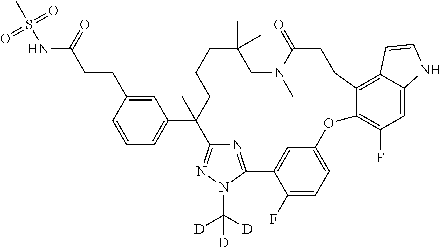 [SMILES: [2H]C([2H])([2H])n1nc2nc1-c1cc(ccc1F)Oc1c(F)cc3[nH]ccc3c1CCC(=O)N(C)CC(C)(C)CCCC2(C)c1cccc(CCC(=O)NS(C)(=O)=O)c1]

To a stirred solution of racemic mixture of 3-[3-[23,29-difluoro-6,10,10,12-tetramethyl-13-oxo-3-(trideuteriomethyl)-25-oxa-3,4,12,20,31-pentazapentacyclo[24.3.1.12,5.016,24.017,21]-hentriaconta-1(30),2(31),4,16,18,21,23,26,28-nonaen-6-yl]phenyl]propanoic acid (1:1 mixture of Examples 77A and 77B, 220 mg, 0.32 mmol) in N,N-dimethylformamide (3 mL) was added methanesulfonamide (61 mg, 0.64 mmol), 1-ethyl-3-(3-dimethylaminopropyl)carbodiimide hydrochloride (123 mg, 0.641 mmol) and N,N-dimethylaminopyridine (78 mg, 0.641 mmol). The mixture was stirred at room temperature overnight, quenched with water (20 mL), and extracted with ethyl acetate (2×20 mL). The combined organic phases were washed with brine, dried over sodium sulfate, and concentrated. The residue was purified by silica gel column chromatography (25 g silica gel column, eluting with 0-10% methanol in dichloromethane) to give the title compound (190 mg, 78%) as a racemic solid. The solid was subjected to chiral Prep-HPLC separation under the following conditions: Instrument: Gilson-281; Column: IE 20*250 mm, 10 μm, Mobile Phase: n-Hexane (0.1% diethylamine): ethanol (0.1% diethylamine)=6:4; Flow Rate: 50 mL/minute; Run time per injection: 25 minutes; Injection volume: 1 mL; Sample solution: 190 mg in 15 mL of methanol. The first eluent, Enantiomer 1, is designated as 79A (42.1 mg, 22%); The second eluent, Enantiomer 2, is designated as 79B (34.71 mg, 18%).

Compound 79A: MS (ESI): 764 m/z [M+H]⁺, retention time: 2.06 minutes, purity: 98% (254 nm) (LC-MS method 4).

Compound 79B: MS (ESI): 764 m/z [M+H]⁺, retention time: 2.06 minutes, purity: 98% (254 nm) (LC-MS method 4).

### Example 80. Compound 80. 3-[3-(23,29-Difluoro-6-methyl-12-oxo-11,25-dioxa-3,13,20,31-tetrazapentacyclo[24.3.1.12,5.016,24.017,21]hentriaconta-1(30),2,4,16,18,21,23,26,28-nonaen-6-yl)-2-fluoro-phenyl]propanoic acid

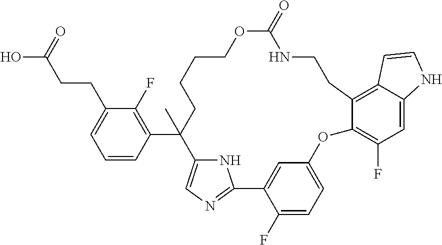 [SMILES: CC1(c2cccc(CCC(=O)O)c2F)CCCCOC(=O)NCCc2c(c(F)cc3[nH]ccc23)Oc2ccc(F)c(c2)-c2ncc1[nH]2]

### Ethyl (E)-3-(5-(3-(5-(6-acetoxy-2-(3-bromo-2-fluorophenyl)hexan-2-yl)-1H-imidazol-2-yl)-4-fluorophenoxy)-6-fluoro-1H-indol-4-yl)acrylate

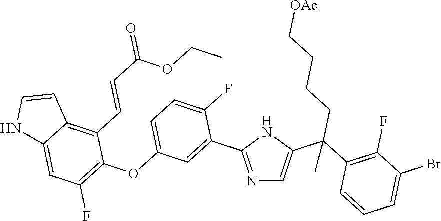 [SMILES: CCOC(=O)/C=C/c1c(Oc2ccc(F)c(-c3ncc(C(C)(CCCCOC(C)=O)c4cccc(Br)c4F)[nH]3)c2)c(F)cc2[nH]ccc12]

Step A: To a stirred solution of ethyl (E)-3-(5-(3-carbamimidoyl-4-fluorophenoxy)-6-fluoro-1H-indol-4-yl)acrylate (Intermediate 5-2, 1.14 mmol, 424 mg) in N,N-dimethylformamide (5 mL) was added 7-bromo-5-(3-bromo-2-fluorophenyl)-5-methyl-6-oxoheptyl acetate (Intermediate 1-17, 500 mg, 1.14 mmol) and sodium bicarbonate (192 mg, 2.28 mmol). The reaction mixture was stirred at 80° C. for 16 hours, cooled to room temperature and quenched with 50 mL of water. The solution was extracted with ethyl acetate (3×20 mL). The combined organic layers were washed with saturated lithium chloride, dried over sodium sulfate and concentrated. The residue was purified by automated flash chromatography (eluting with 0-50% ethyl acetate in petroleum ether) to give the title compound (440 mg, 54%) as a yellow oil. MS (ESI): 724, 726 m/z [M+H]⁺, retention time: 1.81 minutes, purity: 84% (214 nm) (LC-MS method 5).

### (E)-3-(5-(3-(5-(2-(3-Bromo-2-fluorophenyl)-6-hydroxyhexan-2-yl)-1H-imidazol-2-yl)-4-fluorophenoxy)-6-fluoro-1H-indol-4-yl)acrylic acid

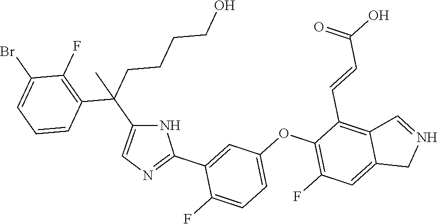 [SMILES: CC(CCCCO)(c1cnc(-c2cc(Oc3c(F)cc4c(c3/C=C/C(=O)O)C=NC4)ccc2F)[nH]1)c1cccc(Br)c1F]

Step B: To a stirred and cooled (0° C.) solution of Step A product (500 mg, 0.78 mmol) in tetrahydrofuran (12 mL) and methanol (4 mL) was added lithium hydroxide monohydrate (326 mg, 7.8 mmol, dissolved in 4 mL of water) dropwise. The solution was warmed to room temperature and stirred for 16 hours, then acidified to pH˜3 with 1 M hydrochloric acid. The mixture was extracted with ethyl acetate (3×50 mL). The combined organic phases were dried over sodium sulfate and concentrated to give the crude title compound (435 mg, 77%) as a yellow solid. MS (ESI): 654, 656 m/z [M+H]⁺, retention time: 1.65 minutes, purity: 98% (214 nm) (LC-MS method 13).

### (E)-3-(6-Fluoro-5-(4-fluoro-3-(5-(2-(2-fluoro-3-((E)-3-methoxy-3-oxoprop-1-en-1-yl)phenyl)-6-hydroxyhexan-2-yl)-1H-imidazol-2-yl)phenoxy)-1H-indol-4-yl)acrylic acid

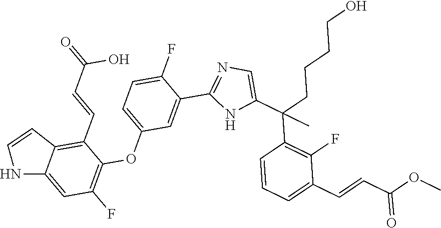 [SMILES: COC(=O)/C=C/c1cccc(C(C)(CCCCO)c2cnc(-c3cc(Oc4c(F)cc5[nH]ccc5c4/C=C/C(=O)O)ccc3F)[nH]2)c1F]

Step C: To a stirred and degassed solution of Step B product (435 mg, 0.67 mmol) in N,N-dimethylformamide (5 mL) was added methyl acrylate (172 mg, 2 mmol), triethylamine (202 mg, 2 mmol), tri(o-tolyl)phosphine (61 mg, 0.2 mmol), followed by palladium(II) acetate (23 mg, 0.1 mmol). The reaction was heated at 130° C. for 5 hours in a microwave reactor. The mixture was diluted with 50 mL of water and extracted with ethyl acetate (2×30 mL). The combined organic layers were washed with water, saturated lithium chloride (30 mL), dried over sodium sulfate and concentrated. The residue was purified by automated flash chromatography (eluting with 0-6% methanol in dichloromethane) gave the title compound (217 mg, 49%) as a brown oil. MS (ESI): 660 m/z [M+H]⁺, retention time: 1.65 minutes, purity: 99% (214 nm) (LC-MS method 5).

### 3-(6-Fluoro-5-(4-fluoro-3-(5-(2-(2-fluoro-3-(3-methoxy-3-oxopropyl)phenyl)-6-hydroxyhexan-2-yl)-1H-imidazol-2-yl)phenoxy)-1H-indol-4-yl)propanoic acid

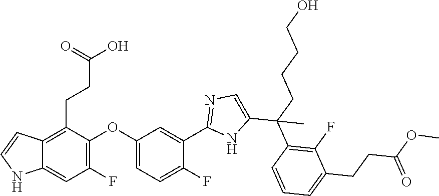 [SMILES: COC(=O)CCc1cccc(C(C)(CCCCO)c2cnc(-c3cc(Oc4c(F)cc5[nH]ccc5c4CCC(=O)O)ccc3F)[nH]2)c1F]

Step D: To a stirred solution of Step C product (217 mg, 0.33 mmol) in ethanol (20 mL) was added palladium on carbon (100 mg, 50% wet, 10%). The reaction mixture was stirred at room temperature for 16 hours under hydrogen balloon, then filtered through a pad of Celite. The filtrate was concentrated to afford the crude title compound (200 mg, 92%) as a brown solid. MS (ESI): 664 m/z [M+H]⁺, retention time: 1.68 minutes, purity: 95% (214 nm) (LC-MS method 3).

### Methyl 3-(3-(2-(2-(5-((4-(3-azido-3-oxopropyl)-6-fluoro-1H-indol-5-yl)oxy)-2-fluorophenyl)-1H-imidazol-5-yl)-6-hydroxyhexan-2-yl)-2-fluorophenyl)propanoate

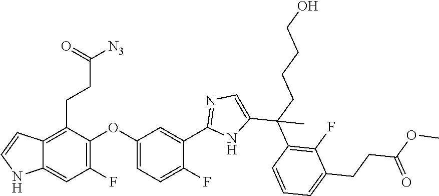 [SMILES: COC(=O)CCc1cccc(C(C)(CCCCO)c2cnc(-c3cc(Oc4c(F)cc5[nH]ccc5c4CCC(=O)N=[N+]=[N-])ccc3F)[nH]2)c1F]

Step E: To a stirred solution of Step D product (200 mg, 0.3 mmol) and triethylamine (30 mg, 0.45 mmol) in tetrahydrofuran (30 mL) was added diphenyl phosphoryl azide (83 mg, 0.45 mol). The mixture was stirred for 16 hours at room temperature and concentrated. The residue was purified by flash chromatography (petroleum ether/ethyl acetate=1:1) to give the title compound (70 mg, 34%) as a yellow oil. MS (ESI): 689 m/z [M+H]⁺, retention time: 1.60 minutes, purity: 86% (214 nm) (LC-MS method 3).

### Methyl 3-[3-(23,29-difluoro-6-methyl-12-oxo-11,25-dioxa-3,13,20,31-tetrazapentacyclo-[24.3.1.12,5.016,24.017,21]hentriaconta-1(30),2,4,16,18,21,23,26,28-nonaen-6-yl)-2-fluoro-phenyl]propanoate

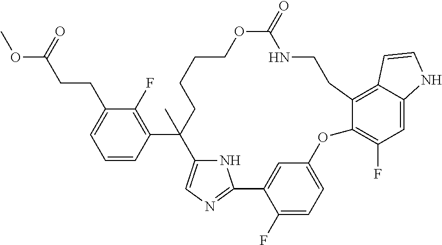 [SMILES: COC(=O)CCc1cccc(C2(C)CCCCOC(=O)NCCc3c(c(F)cc4[nH]ccc34)Oc3ccc(F)c(c3)-c3ncc2[nH]3)c1F]

Step F: A mixture of Step E product (70 mg, 0.1 mol) and triethylamine (21 mg, 0.2 mol) in toluene (30 mL) was heated at 110° C. for 16 hours and concentrated. The residue was purified by silica gel column chromatography (petroleum ether/ethyl acetate=2:1) to give the title compound (12 mg, 18%) as a white solid. MS (ESI): 661 m/z [M+H]⁺, retention time: 1.79 minutes, purity: 89% (214 nm) (LC-MS method 14).

### Compound 80: 3-[3-(23,29-Difluoro-6-methyl-12-oxo-11,25-dioxa-3,13,20,31-tetrazapentacyclo[24.3.1.12,5.016,24.017,21]hentriaconta-1(30),2,4,16,18,21,23,26,28-nonaen-6-yl)-2-fluoro-phenyl]propanoic acid

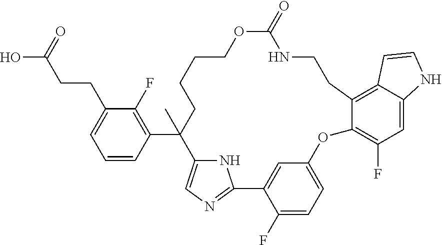 [SMILES: CC1(c2cccc(CCC(=O)O)c2F)CCCCOC(=O)NCCc2c(c(F)cc3[nH]ccc23)Oc2ccc(F)c(c2)-c2ncc1[nH]2]

Step G: To a stirred and cooled (0° C.) solution of Step F product (12 mg, 0.02 mmol) in tetrahydrofuran (3 mL) and methanol (1 mL) was added lithium hydroxide monohydrate (8 mg, 0.2 mmol, dissolved in 1 mL of water) dropwise. The solution was stirred at room temperature for 3 hours and concentrated. The residue was dissolved in 1 mL of water, acidified to pH˜6 with 1 M hydrochloric acid, and extracted with ethyl acetate (2×25 mL). The combined organic layers were washed with water, brine, dried over sodium sulfate and concentrated. The residue was purified by Prep-HPLC (ammonium bicarbonate as additive) to give the title compound (2 mg, 15%) as white solid. MS (ESI): 647 m/z [M+H]⁺, retention time: 1.36 minutes, purity: >99% (214 nm) (LC-MS method 3). ¹HNMR (400 MHz, CD₃OD) δ 7.64 (s, 1H), 7.28-7.09 (m, 8H), 6.52 (s, 1H), 4.17-4.03 (m, 1H), 3.91-3.73 (m, 1H), 3.11-2.84 (m, 5H), 2.62-2.33 (m, 3H), 2.19-2.03 (m, 1H), 1.76-1.24 (m, 7H), 1.13-1.00 (m, 1H) ppm.

### Example 81. Compound 81. 3-[3-(23,29-Difluoro-12-oxo-11,25-dioxa-3,13,20,31-tetrazapentacyclo[24.3.1.12,5.016,24.017,21]hentriaconta-1(30),2,4,16(24),17(21),18,22,26,28-nonaen-6-yl)-2-fluoro-phenyl]propanoic acid

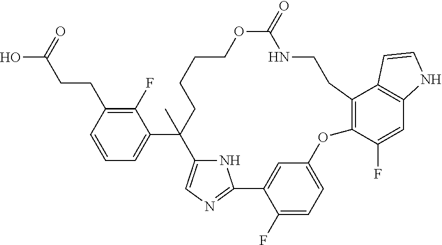 [SMILES: CC1(c2cccc(CCC(=O)O)c2F)CCCCOC(=O)NCCc2c(c(F)cc3[nH]ccc23)Oc2ccc(F)c(c2)-c2ncc1[nH]2]

### Example 82. Compound 82. 23,29-Difluoro-6-(2-fluorophenyl)-11,25-dioxa-3,13,20,31-tetrazapentacyclo[24.3.1.12,5.016,24.017,21]hentriaconta-1(30),2,4,16(24),17(21),18,22,26,28-nonaen-12-one

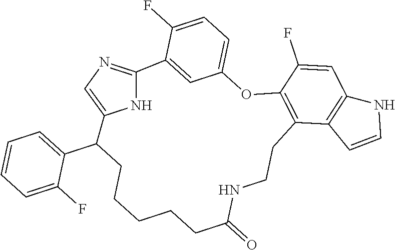 [SMILES: O=C1CCCCCC(c2ccccc2F)c2cnc([nH]2)-c2cc(ccc2F)Oc2c(F)cc3[nH]ccc3c2CCN1]

Exchanging ethyl (E)-3-(5-(3-carbamimidoyl-4-fluorophenoxy)-6-fluoro-1H-indol-4-yl)acrylate (Intermediate 5-2) with methyl (E)-3-(5-(3-carbamimidoyl-4-fluorophenoxy)-6-fluoro-1H-indol-4-yl)acrylate (Intermediate 5, 1.57 g, 0.00424 mol) and 7-bromo-5-(3-bromo-2-fluorophenyl)-5-methyl-6-oxoheptyl acetate (Intermediate 1-17) with 7-bromo-5-(3-bromo-2-fluorophenyl)-6-oxoheptyl acetate (intermediate 1-18, 1.8 g, 0.00424 mol), the 7 step reaction procedure sequence (Steps A to G) described for Example 80 was used to prepare the title compounds 81 (58 mg, white solid) and 82 (4.5 mg, white solid). 82 originated from the de-bromination product in corresponding Step C, Heck reaction, and carried on as a mixture and separated at the last step.

Compound 81: MS (ESI): 633 m/z [M+H]⁺, retention time: 1.56 minutes, purity: >99% (214 nm) (LC-MS method 14). ¹H NMR (400 MHz, CD₃OD) δ 7.63 (s, 1H), 7.30-7.29 (m, 3H), 7.22-7.15 (m, 3H), 7.06-7.04 (m, 1H), 6.71 (s, 1H), 6.58 (s, 1H), 4.31-4.29 (m, 1H), 4.05-4.03 (m, 1H), 3.93-3.92 (m, 1H), 3.43-3.39 (m, 1H), 3.13-3.11 (m, 2H), 2.98-2.93 (m, 2H), 2.61-2.55 (m, 2H), 2.27-2.24 (m, 1H), 1.85-1.80 (m, 1H), 1.70-1.69 (m, 1H), 1.38-1.31 (m, 4H) ppm.

Compound 82: MS (ESI): 561 m/z [M+H]⁺, retention time: 1.80 minutes, purity: >99% (214 nm) (LC-MS method 14). ¹H NMR (400 MHz, CD₃OD) δ 7.63 (s, 1H), 7.33-7.32 (m, 1H), 7.20-7.15 (m, 1H), 7.10-6.98 (m, 4H), 6.95-6.93 (m, 2H), 6.57 (s, 1H), 6.46 (s, 1H), 4.19-4.15 (m, 1H), 3.96-3.94 (m, 1H), 3.84-3.82 (m, 1H), 3.39-3.34 (m, 1H), 3.03-2.99 (m, 2H), 2.18-2.07 (m, 1H), 1.74-1.71 (m, 1H), 1.64-1.57 (m, 1H), 1.36-1.33 (m, 4H) ppm.

### Example 83. Compound 83. 3-[3-(22,28-Difluoro-6-methyl-9-oxo-10,24-dioxa-3,8,19,30-tetrazapentacyclo[23.3.1.12,5.015,23.016,20]triaconta-1(29),2,4,15,17,20,22,25,27-nonaen-6-yl)phenyl]propanoic acid

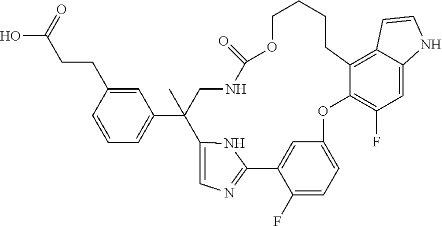 [SMILES: CC1(c2cccc(CCC(=O)O)c2)CNC(=O)OCCCCc2c(c(F)cc3[nH]ccc23)Oc2ccc(F)c(c2)-c2ncc1[nH]2]

### 3-(2-(2-Fluoro-5-((6-fluoro-4-(4-hydroxybutyl)-1H-indol-5-yl)oxy)phenyl)-1H-imidazol-4-yl)-3-(3-iodophenyl)butanenitrile

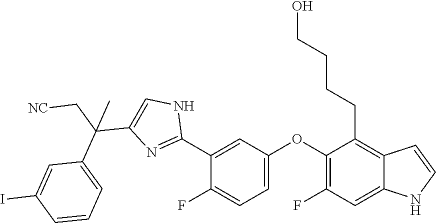 [SMILES: CC(CC#N)(c1cccc(I)c1)c1c[nH]c(-c2cc(Oc3c(F)cc4[nH]ccc4c3CCCCO)ccc2F)n1]

Step A: To a stirred solution of 2-fluoro-5-((6-fluoro-4-(4-hydroxybutyl)-1H-indol-5-yl)oxy)benzimidamide (Intermediate 5-10, 700 mg, 1.95 mmol) in dry N,N-dimethylformamide (10 mL) was added sodium bicarbonate (327 mg, 3.90 mmol) and 5-chloro-3-(3-iodophenyl)-3-methyl-4-oxopentanenitrile (Intermediate 1-15, 677 mg, 1.95 mmol). The mixture was stirred at 75° C. for 16 hours, cooled to room temperature, and quenched with 50 mL of brine. The solid was collected by filtration, and dried. The crude product was purified by column chromatography (eluting with petroleum ether/ethyl acetate=2/1) to give the title compound (760 mg, 59%) as a yellow solid. MS (ESI): 653 m/z [M+H]⁺, retention time: 1.87 minutes, purity: 96% (214 nm) (LC-MS method 15).

### 3-(2-(2-Fluoro-5-((6-fluoro-4-(4-hydroxybutyl)-1H-indol-5-yl)oxy)phenyl)-1H-imidazol-4-yl)-3-(3-iodophenyl)butanoic acid

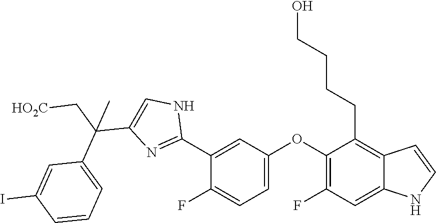 [SMILES: CC(CC(=O)O)(c1cccc(I)c1)c1c[nH]c(-c2cc(Oc3c(F)cc4[nH]ccc4c3CCCCO)ccc2F)n1]

Step B: A mixture of Step A product (760 mg, 1.17 mmol) in ethanol (20 mL) and hydrochloric acid (6 M, 20 mL) was stirred at 110° C. for two days and concentrated. The residue was dissolved in ethanol (20 mL) and water (10 mL), and then added potassium hydroxide (652 mg, 11.66 mmol). The mixture was heated at 50° C. for 16 hours, then concentrated to remove ethanol. The residue was acidified to pH˜3 with 1 N hydrochloric acid and extracted with ethyl acetate (3×30 mL). The combined organic phases were washed with brine, dried over sodium sulfate, and concentrated. The crude product was purified by silica gel column chromatography (eluted with dichloromethane/methanol/acetic acid=30/1/0.01) to give the title compound (310 mg, 43%) as a yellow solid. MS (ESI): 672 m/z [M+H]⁺, retention time: 1.78 minutes, purity: 97% (214 nm) (LC-MS method 5).

### Compound 83. 3-[3-(22,28-difluoro-6-methyl-9-oxo-10,24-dioxa-3,8,19,30-tetrazapentacyclo[23.3.1.12,5.015,23.016,20]triaconta-1(29),2,4,15,17,20,22,25,27-nonaen-6-yl)phenyl]propanoic acid

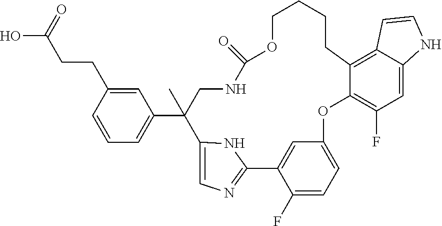 [SMILES: CC1(c2cccc(CCC(=O)O)c2)CNC(=O)OCCCCc2c(c(F)cc3[nH]ccc23)Oc2ccc(F)c(c2)-c2ncc1[nH]2]

Step C: Exchanging (E)-3-(5-(3-(5-(2-(3-Bromo-2-fluorophenyl)-6-hydroxyhexan-2-yl)-1H-imidazol-2-yl)-4-fluorophenoxy)-6-fluoro-1H-indol-4-yl)acrylic acid (Step B product of Example 80) with 3-(2-(2-Fluoro-5-((6-fluoro-4-(4-hydroxybutyl)-1H-indol-5-yl)oxy)phenyl)-1H-imidazol-4-yl)-3-(3-iodophenyl)butanoic acid (Step B product of this example, 310 mg, 0.46 mmol), the reaction procedure sequence (Steps C to G) described for Example 80 was used to prepare the title compound (6.1 mg) as a white solid. MS (ESI): 615 m/z [M+H]⁺, retention time: 1.90 minutes, purity: 97% (214 nm) (LC-MS method 13). ¹H NMR (400 MHz, CD₃OD) δ 7.28 (d, J=3.2 Hz, 1H), 7.23 (t, J=7.6 Hz, 1H), 7.16-7.04 (m, 6H), 6.95-6.91 (m, 2H), 6.54 (d, J=2.4 Hz, 1H), 3.83 (d, J=13.2 Hz, 1H), 3.61 (d, J=13.2 Hz, 1H), 3.52 (t, J=6.8 Hz, 2H), 2.92-2.87 (m, 4H), 2.55-2.51 (m, 2H), 1.72-1.68 (m, 5H), 1.60-1.55 (m, 2H) ppm.

### Example 84. Compound 84. 3-[3-(23,29-Difluoro-6,12-dimethyl-11-oxo-10,25-dioxa-3,12,20,31-tetrazapentacyclo[24.3.1.12,5.016,24.017,21]hentriaconta-1(30),2,4,16(24),17(21),18,22,26,28-nonaen-6-yl)phenyl]propanoic acid

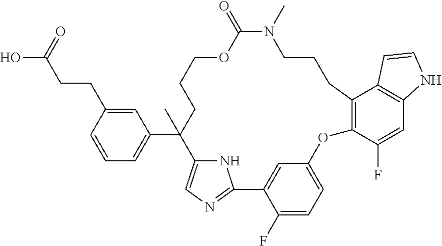 [SMILES: CN1CCCc2c(c(F)cc3[nH]ccc23)Oc2ccc(F)c(c2)-c2ncc([nH]2)C(C)(c2cccc(CCC(=O)O)c2)CCCOC1=O]

### Ethyl 3-(3-(5-acetoxy-2-(2-(5-((4-bromo-6-fluoro-1H-indol-5-yl)oxy)-2-fluorophenyl)-1H-imidazol-5-yl)pentan-2-yl)phenyl)propanoate

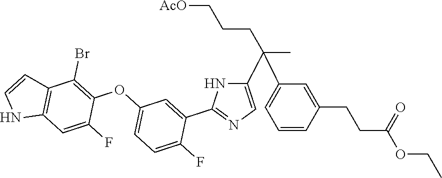 [SMILES: CCOC(=O)CCc1cccc(C(C)(CCCOC(C)=O)c2cnc(-c3cc(Oc4c(F)cc5[nH]ccc5c4Br)ccc3F)[nH]2)c1]

Step A: To a stirred solution of ethyl 3-(3-(6-acetoxy-1-bromo-3-methyl-2-oxohexan-3-yl)phenyl) propanoate (Intermediate 1-20, 600 mg, 1.4 mmol) in N,N-dimethylformamide (8 mL) was added 5-((4-bromo-6-fluoro-1H-benzo[d]imidazol-5-yl)oxy)-2-fluorobenzimidamide (Intermediate 5-11, 514 mg, 1.4 mmol) and sodium bicarbonate (352 mg, 4.2 mmol). The mixture was stirred at 60° C. overnight, cooled to room temperature, and diluted with ethyl acetate (60 mL). The solution was washed with water, brine, dried over sodium sulfate, and concentrated. The residue was purified by silica gel column chromatography (eluting with petroleum ether/dichloromethane/ethyl acetate=3/2/2) to afford the title compound (560 mg, 58%) as a white solid. MS (ESI): 694, 696 m/z [M+H]⁺, retention time: 1.73 minutes, purity: 91% (214 nm) (LC-MS method 3).

### Ethyl 3-(3-(2-(2-(5-((4-bromo-6-fluoro-1H-indol-5-yl)oxy)-2-fluorophenyl)-1H-imidazol-5-yl)-5-hydroxypentan-2-yl)phenyl)propanoate

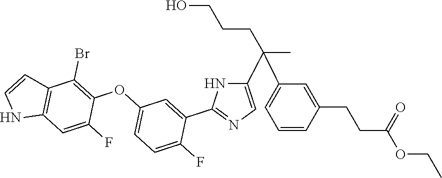 [SMILES: CCOC(=O)CCc1cccc(C(C)(CCCO)c2cnc(-c3cc(Oc4c(F)cc5[nH]ccc5c4Br)ccc3F)[nH]2)c1]

Step B: To a stirred solution of Step B product (560 mg, 0.8 mmol) in ethanol (6 mL) was added potassium carbonate (883 mg, 6.4 mmol). The mixture was stirred at 60° C. for 18 hours, cooled to room temperature, and diluted with ethyl acetate (60 mL). The solution was washed with water, brine, dried over sodium sulfate, and concentrated. The residue was purified by silica gel column chromatography (eluting with petroleum ether/ethyl acetate=3/2) to give the title compound (400 mg, 76%) as a white solid. MS (ESI): 652, 654 m/z [M+H]⁺, retention time: 1.67 minutes, purity: 91% (214 nm) (LC-MS method 3).

### Ethyl 3-(3-(2-(2-(5-((4-bromo-6-fluoro-1H-indol-5-yl)oxy)-2-fluorophenyl)-1H-imidazol-5-yl)-5-(((4-nitrophenoxy)carbonyl)oxy)pentan-2-yl)phenyl)propanoate

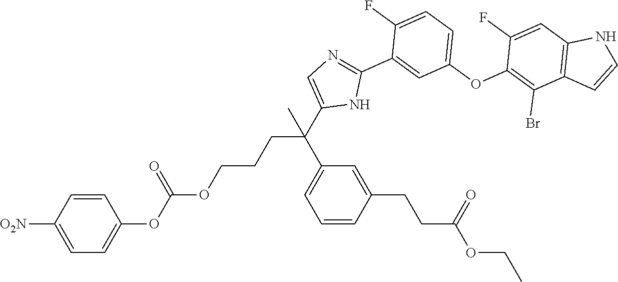 [SMILES: CCOC(=O)CCc1cccc(C(C)(CCCOC(=O)Oc2ccc([N+](=O)[O-])cc2)c2cnc(-c3cc(Oc4c(F)cc5[nH]ccc5c4Br)ccc3F)[nH]2)c1]

Step C: To a stirred solution of Step B product (380 mg, 0.582 mmol) in dichloromethane (40 mL) was added triethylamine (327 μL, 2.34 mmol) followed by (4-nitrophenyl) carbonochloridate (236 mg, 1.17 mmol). The mixture was stirred at room temperature overnight, washed with water, brine, dried over sodium sulfate and concentrated. The residue was purified by automated flash chromatography (12 g silica gel column, eluting with 0-80% ethyl acetate in petroleum ether) to give the title compound (230 mg, 48%) as a solid. MS (ESI): 817, 819 m/z [M+H]⁺, retention time: 1.90 minutes, purity: 94% (214 nm) (LC-MS method 3).

### Ethyl (E)-3-(3-(2-(2-(5-((4-bromo-6-fluoro-1H-indol-5-yl)oxy)-2-fluorophenyl)-1H-imidazol-5-yl)-5-((methyl(3-(tributylstannyl)allyl)carbamoyl)oxy)pentan-2-yl)phenyl)propanoate

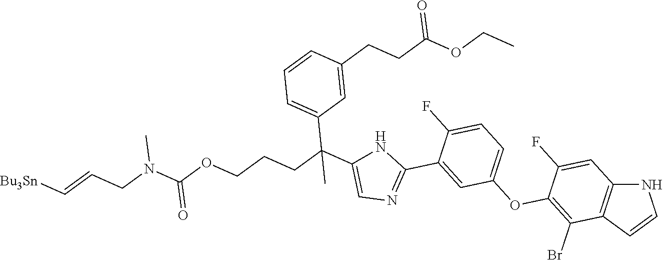

Step D: To a stirred solution of Step C product (230 mg, 0.281 mmol) and (E)-N-methyl-3-(tributylstannyl)prop-2-en-1-amine (Intermediate 36, 152 mg, 0.421 mmol) in N,N-dimethylformamide (10 mL) was added diisopropylethylamine (97 μL, 0.587 mmol). The mixture was stirred at room temperature overnight, then diluted with ethyl acetate (50 mL). The solution was washed with brine, dried over sodium sulfate, and concentrated. The residue was purified by automated flash chromatography (12 g silica gel column, eluting with 0-80% ethyl acetate in petroleum ether) to give the title compound (240 mg, 82%) as a solid. MS (ESI): 1039 m/z [M+H]⁺, retention time: 2.39 minutes, purity: 90% (214 nm) (LC-MS method 7).

### Ethyl 3-[3-[(14E)-23,29-difluoro-6,12-dimethyl-11-oxo-10,25-dioxa-3,12,20,31-tetrazapentacyclo[24.3.1.12,5.016,24.017,21]hentriaconta-1(30),2,4,14,16(24),17(21),18,22,26,28-decaen-6-yl]phenyl]propanoate

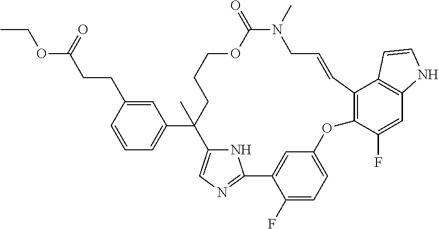 [SMILES: CCOC(=O)CCc1cccc(C2(C)CCCOC(=O)N(C)C/C=C/c3c(c(F)cc4[nH]ccc34)Oc3ccc(F)c(c3)-c3ncc2[nH]3)c1]

Step E: In a glovebox, to a stirred solution of Step D product (240 mg, 0.231 mmol) in toluene (20 mL) was added tris(dibenzylideneacetone)dipalladium(0) (CAS: 60748-47-2) (48 mg) and JackiePhos (CAS: 1160861-60-8) (48 mg) in toluene (20 mL). The mixture was stirred at 60° C. overnight and concentrated. The residue was purified by automated flash chromatography (12 g silica gel column, eluting with 0-80% ethyl acetate in petroleum ether) to give the title compound (102 mg, 66%) as a solid. MS (ESI): 669 m/z [M+H]⁺, retention time: 1.81 minutes, purity: 99% (214 nm) (LC-MS method 7).

### Ethyl 3-[3-(23,29-difluoro-6,12-dimethyl-11-oxo-10,25-dioxa-3,12,20,31-tetrazapentacyclo-[24.3.1.12,5.016,24.017,21]hentriaconta-1(30),2,4,16(24),17(21),18,22,26,28-nonaen-6-yl)phenyl]propanoate

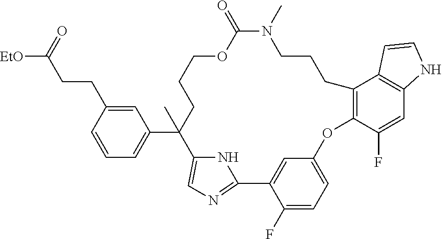 [SMILES: CCOC(=O)CCc1cccc(C2(C)CCCOC(=O)N(C)CCCc3c(c(F)cc4[nH]ccc34)Oc3ccc(F)c(c3)-c3ncc2[nH]3)c1]

Step F: To a stirred solution of Step E product (102 mg, 0.153 mmol) in ethyl acetate (20 mL) was added 10% palladium on carbon (50% wet, 50 mg). The reaction was stirred at 40° C. under hydrogen balloon for 7 hours. Another portion of 10% palladium on carbon (50% wet, 50 mg) was added and stirred at 40° C. under hydrogen balloon for additional 3 hours. The mixture was filtered through a pad of celite. The filter cake was rinsed with ethyl acetate (2×10 mL). The combined filtrate was concentrated. The residue was purified by automated flash chromatography (40 g silica gel column, eluting with 0-80% ethyl acetate in petroleum ether) to give the title compound (74 mg, 72%) as a solid. MS (ESI): 671 m/z [M+H]⁺, retention time: 1.91 minutes, purity: 99% (254 nm) (LC-MS method 7).

### Compound 84: 3-[3-(23,29-Difluoro-6,12-dimethyl-]1-oxo-10,25-dioxa-3,12,20,31-tetrazapentacyclo[24.3.1.12,5.016,24.017,21]hentriaconta-1(30),2,4,16(24),17(21),18,22,26,28-nonaen-6-yl)phenyl]propanoic acid

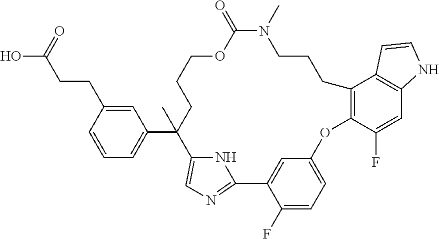 [SMILES: CN1CCCc2c(c(F)cc3[nH]ccc23)Oc2ccc(F)c(c2)-c2ncc([nH]2)C(C)(c2cccc(CCC(=O)O)c2)CCCOC1=O]

Step G: To a stirred solution of Step F product (82 mg, 0.122 mmol) in tetrahydrofuran (6 mL) and methanol (2 mL) was added a solution of lithium hydroxide monohydrate (84 mg, 2 mmol) in water (2 mL). The mixture was stirred at room temperature for 4 hours, then acidified with 2 M hydrochloric acid to pH˜5 and diluted with ethyl acetate (50 mL). The separated organic layer was washed with brine, dried over sodium sulfate, and concentrated to afford the title compound (52.1 mg, 66%) as a white solid. MS (ESI): 643 m/z [M+H]⁺, retention time: 1.71 minutes, purity: 99% (254 nm) (LC-MS method 7). ¹H NMR (400 MHz, CD₃OD) δ 7.78-7.38 (m, 1H), 7.27 (s, 1H), 7.23-7.09 (m, 5H), 7.01-6.94 (m, 3H), 6.51 (s, 1H), 3.96-3.88 (m, 2H), 3.51-3.41 (m, 1H), 3.20-3.15 (m, 1H), 2.92-2.82 (m, 7H), 2.55-2.40 (m, 3H), 2.19-1.62 (m, 5H), 1.58 (s, 3H) ppm.

### Example 85. Compound 85. 3-[3-(23,29-Difluoro-13-oxo-14,25-dioxa-3,12,20,31-tetrazapentacyclo[24.3.1.12,5.016,24.017,21]hentriaconta-1(30),2,4,16,18,21,23,26,28-nonaen-6-yl)-2-fluoro-phenyl]propanoic acid

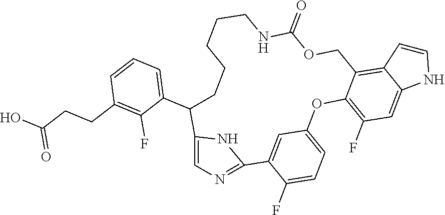 [SMILES: O=C(O)CCc1cccc(C2CCCCCNC(=O)OCc3c(c(F)cc4[nH]ccc34)Oc3ccc(F)c(c3)-c3ncc2[nH]3)c1F]

Exchanging 2-fluoro-5-((6-fluoro-4-(4-hydroxybutyl)-1H-indol-5-yl)oxy)benzimidamide (Intermediate 5-10) with 2-fluoro-5-((6-fluoro-4-(hydroxymethyl)-1H-indol-5-yl)oxy)benzimidamide (intermediate 5-5, 1.72 g, 5.43 mmol), and 5-chloro-3-(3-iodophenyl)-3-methyl-4-oxopentanenitrile (Intermediate 1-15) with 9-bromo-7-(3-bromo-2-fluorophenyl)-8-oxononanenitrile (Intermediate 1-21, 2.2 g, 5.43 mmol), the reaction procedure sequence (Steps A, B of Example 83, followed by Steps C to G of Example 80) was used to prepare the title compound (14.2 mg) as a white solid, with slight modification. The corresponding Step B of Example 83, hydrolysis of nitrile, was done with sodium hydroxide/ethanol/water only, without first treatment with hydrochloric acid/ethanol. MS (ESI): 633 m/z [M+H]⁺, retention time: 1.39 minutes, purity: >99% (254 nm) (LC-MS method 4). ¹H NMR (400 MHz, DMSO-d₆) δ 11.97 (s, 1H), 11.35 (s, 1H), 7.64 (s, 1H), 7.45 (s, 1H), 7.38-7.32 (m, 1H), 7.27-7.23 (m, 2H), 7.12-6.93 (m, 4H), 6.90-6.86 (m, 1H), 6.73-6.69 (m, 1H), 5.30-5.18 (m, 2H), 4.21-4.15 (m, 1H), 2.31-2.96 (m, 1H), 2.84-2.73 (m, 3H), 2.50-2.42 (m, 2H), 1.42-1.22 (m, 8H) ppm.

### Example 86. Compound 86. 3-[3-(23,29-Difluoro-6-methyl-12-oxo-13,25-dioxa-3,11,20,31-tetrazapentacyclo[24.3.1.12,5.016,24.017,21]hentriaconta-1(30),2,4,16(24),17(21),18,22,26,28-nonaen-6-yl)phenyl]propanoic acid

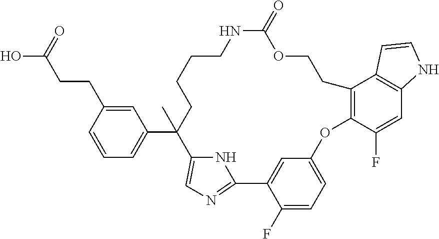 [SMILES: CC1(c2cccc(CCC(=O)O)c2)CCCCNC(=O)OCCc2c(c(F)cc3[nH]ccc23)Oc2ccc(F)c(c2)-c2ncc1[nH]2]

Exchanging 2-fluoro-5-((6-fluoro-4-(4-hydroxybutyl)-1H-indol-5-yl)oxy)benzimidamide (Intermediate 5-10) with 2-fluoro-5-((6-fluoro-4-(2-hydroxyethyl)-1H-indol-5-yl)oxy)benzimidamide (intermediate 5-12, 1.56 g, 4.7 mmol), and 5-chloro-3-(3-iodophenyl)-3-methyl-4-oxopentanenitrile (Intermediate 1-15) with 8-bromo-6-(3-iodophenyl)-6-methyl-7-oxooctanenitrile (Intermediate 1-11, 1.7 g, 3.9 mmol), the reaction procedure sequence (Steps A, B of Example 83, followed by Steps C to G of Example 80) was used to prepare the title compound (11 mg) as a white solid, with slight modification. The corresponding Step B hydrolysis of nitrile was done with sodium hydroxide/ethanol/water only, without first treatment with hydrochloric acid/ethanol. MS (ESI): 629 m/z [M+H]⁺, retention time: 1.70 minutes, purity: >99% (254 nm) (LC-MS method 9). ¹H NMR (400 MHz, CD₃OD) δ 7.26-7.34 (m, 2H), 7.12-7.22 (m, 4H), 6.97-7.07 (m, 4H), 6.53 (s, 1H), 4.02-4.20 (m, 2H), 3.18-3.12 (m, 3H), 2.88-2.80 (m, 3H), 2.40 (t, J=7.2 Hz, 2H), 2.34-2.21 (m, 1H), 1.83-1.92 (m, 1H), 1.58 (s, 3H), 1.25-1.45 (m, 3H), 1.08-0.95 (m, 1H) ppm.

### Example 87. Compound 87. 3-[3-(23,29-Difluoro-6,9-dimethyl-12-oxo-11,25-dioxa-3,13,20,31-tetrazapentacyclo[24.3.1.12,5.016,24.017,21]hentriaconta-1(30),2,4,16(24),17(21),18,22,26,28-nonaen-6-yl)phenyl]propanoic acid

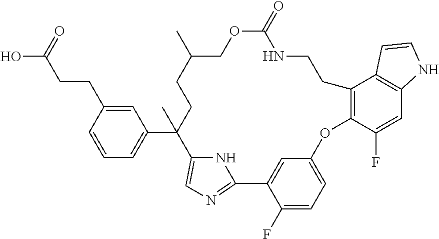 [SMILES: CC1CCC(C)(c2cccc(CCC(=O)O)c2)c2cnc([nH]2)-c2cc(ccc2F)Oc2c(F)cc3[nH]ccc3c2CCNC(=O)OC1]

Exchanging ethyl (E)-3-(5-(3-carbamimidoyl-4-fluorophenoxy)-6-fluoro-1H-indol-4-yl)acrylate (Intermediate 5-2) with methyl (E)-3-(5-(3-carbamimidoyl-4-fluorophenoxy)-6-fluoro-1H-indol-4-yl)acrylate (intermediate 5, 1 g, 2.695 mmol), and 7-bromo-5-(3-bromo-2-fluorophenyl)-5-methyl-6-oxoheptyl acetate (Intermediate 1-17) with 7-bromo-5-(3-iodophenyl)-2,5-dimethyl-6-oxoheptyl acetate (Intermediate 1-30, 1.08 g, 2.246 mmol), the reaction procedure sequence (Steps A to G) described for Example 80 was used to prepare the title compound (57 mg) as a white solid. MS (ESI): 643 m/z [M+H]⁺, retention time: 1.50 minutes, purity: >99% (254 nm) (LC-MS method 9). (Mixture of four diastereomers, originated from two chiral center).

### Example 88. Compound 88. 3-[3-(23,29-Difluoro-6,11,11-trimethyl-13-oxo-14,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

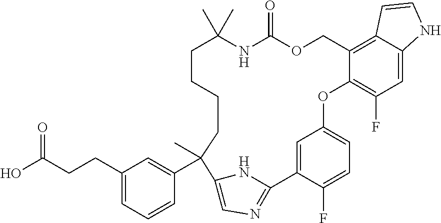 [SMILES: CC1(C)CCCCC(C)(c2cccc(CCC(=O)O)c2)c2cnc([nH]2)-c2cc(ccc2F)Oc2c(F)cc3[nH]ccc3c2COC(=O)N1]

Exchanging ethyl (E)-3-(5-(3-carbamimidoyl-4-fluorophenoxy)-6-fluoro-1H-indol-4-yl)acrylate (Intermediate 5-2) with 2-fluoro-5-((6-fluoro-4-(hydroxymethyl)-1H-indol-5-yl)oxy)benzimidamide (intermediate 5-5, 750 mg, 2.36 mmol), and 7-bromo-5-(3-bromo-2-fluorophenyl)-5-methyl-6-oxoheptyl acetate (Intermediate 1-17) with methyl 9-bromo-7-(3-iodophenyl)-2,2,7-trimethyl-8-oxononanoate (Intermediate 1-31, 1.20 g, 2.36 mmol), the reaction procedure sequence (Steps A to G) described for Example 80 was used to prepare the title compound (1.9 mg) as a white solid. MS (ESI): 657 m/z [M+H]⁺, retention time: 1.47 minutes, purity: >99% (254 nm) (LC-MS method 4). ¹H NMR (400 MHz, CD₃OD) δ 7.53 (s, 1H), 7.32 (d, J=3.2 Hz, 1H), 7.29-7.25 (m, 1H), 7.18-7.12 (m, 3H), 7.09-6.99 (m, 3H), 6.94 (s, 1H), 6.63 (s, 1H), 5.27 (d, J=11.6 Hz, 1H), 5.07 (d, J=11.6 Hz, 1H), 2.90-2.83 (m, 2H), 2.54-2.48 (m, 2H), 2.23-2.16 (m, 2H), 1.54 (s, 3H), 1.37-1.29 (m, 6H), 1.121-1.15 (m, 6H) ppm.

### Example 89. Compound 89. 3-[3-(23,29-Difluoro-6,10,10-trimethyl-13-oxo-12,25-dioxa-3,14,20,31-tetrazapentacyclo[24.3.1.12,5.016,24.017,21]hentriaconta-1(30),2,4,16,18,21,23,26,28-nonaen-6-yl)phenyl]propanoic acid

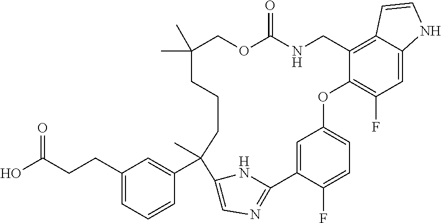 [SMILES: CC1(C)CCCC(C)(c2cccc(CCC(=O)O)c2)c2cnc([nH]2)-c2cc(ccc2F)Oc2c(F)cc3[nH]ccc3c2CNC(=O)OC1]

Exchanging ethyl (E)-3-(5-(3-carbamimidoyl-4-fluorophenoxy)-6-fluoro-1H-indol-4-yl)acrylate (Intermediate 5-2) with ethyl 2-(5-(3-carbamimidoyl-4-fluorophenoxy)-6-fluoro-1H-indol-4-yl)acetate (intermediate 6, 370 mg, 1.0 mmol), and 7-bromo-5-(3-bromo-2-fluorophenyl)-5-methyl-6-oxoheptyl acetate (Intermediate 1-17) with 8-bromo-6-(3-iodophenyl)-2,2,6-trimethyl-7-oxooctyl acetate (intermediate 1-35, 500 mg, 1.0 mmol), the reaction procedure sequence (Steps A to G) described for Example 80 was used to prepare the title compound (3.5 mg) as a white solid. MS (ESI): 657 m/z [M+H]⁺, retention time: 1.55 minutes, purity: >99% (214 nm) (LC-MS method 4). ¹H NMR (400 MHz, CD₃OD) δ 7.29-7.25 (m, 1H), 7.20 (d, J=3.2 Hz, 1H), 7.13-7.08 (m, 2H), 7.05 (d, J=10.2 Hz, 1H), 6.98-6.90 (m, 3H), 6.89-6.80 (m, 2H), 6.50 (d, J=3.1 Hz, 1H), 4.62 (d, J=13 Hz, 1H), 4.23 (d, J=13 Hz, 1H), 3.60-3.54 (m, 1H), 3.17-3.10 (m, 1H), 2.69-2.65 (m, 2H), 2.29-2.25 (m, 2H), 2.13-2.07 (m, 1H), 1.85-1.79 (m, 1H), 1.38 (s, 3H), 1.23-1.18 (m, 2H), 1.07-1.00 (m, 2H), 0.76 (s, 3H), 0.61 (s, 3H) ppm.

### Example 90. Compound 90 3-[3-(23,29-Difluoro-6,10,10-trimethyl-13-oxo-14,25-dioxa-3,4,12,20,31-pentazapentacyclo[24.3.1.12,5.016,24.017,21]hentriaconta-1(30),2,4,16(24),17(21),18,22,26,28-nonaen-6-yl)phenyl]propanoic acid

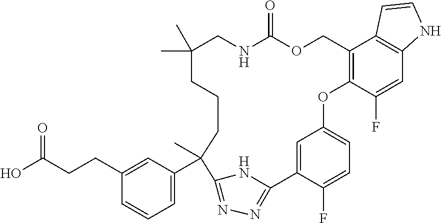 [SMILES: CC1(C)CCCC(C)(c2cccc(CCC(=O)O)c2)c2nnc([nH]2)-c2cc(ccc2F)Oc2c(F)cc3[nH]ccc3c2COC(=O)NC1]

### Example 91. Compound 91. 23,29-Difluoro-6,10,10-trimethyl-6-phenyl-14,25-dioxa-3,4,12,20,31-pentazapentacyclo[24.3.1.12,5.016,24.017,21]hentriaconta-1(30),2,4,16(24),17(21),18,22,26,28-nonaen-13-one

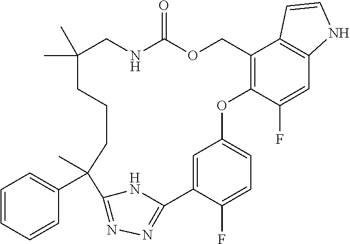 [SMILES: CC1(C)CCCC(C)(c2ccccc2)c2nnc([nH]2)-c2cc(ccc2F)Oc2c(F)cc3[nH]ccc3c2COC(=O)NC1]

### 7-(3-Bromophenyl)-7-(5-(2-fluoro-5-((6-fluoro-4-(hydroxymethyl)-1H-indol-5-yl)oxy)phenyl)-4H-1,2,4-triazol-3-yl)-3,3-dimethyloctanoic acid

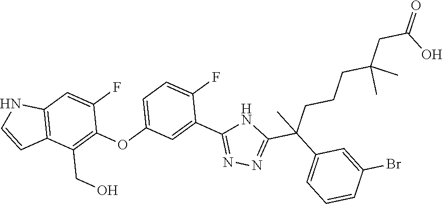 [SMILES: CC(C)(CCCC(C)(c1cccc(Br)c1)c1nnc(-c2cc(Oc3c(F)cc4[nH]ccc4c3CO)ccc2F)[nH]1)CC(=O)O]

Step A: To a stirred solution of 2-fluoro-5-((6-fluoro-4-(hydroxymethyl)-1H-indol-5-yl)oxy)benzimidamide (Intermediate 5-5, 950 mg, 2.99 mmol) in methanol (5 mL) was added 7-(3-bromophenyl)-8-hydrazineyl-3,3,7-trimethyl-8-oxooctanoic acid (Intermediate 56, 1.73 g, 4.49 mmol) and sodium methoxide (647 mg, 12 mmol). The mixture was stirred at 100° C. for 2 hours in microwave reactor, and concentrated. The residue was diluted with water (30 mL), acidified to pH˜5-6 with 1 N hydrochloric acid, and extracted with ethyl acetate (3×30 mL). The combined organic extracts were washed with water, brine, dried over sodium sulfate, and concentrated. The residue was purified by automated flash chromatography (12 g silica gel column, eluting with 0-20% methanol in dichloromethane) to give the title compound (425 mg. 21%) as gray solid. MS (ESI): 667, 669 m/z [M+H]⁺, retention time: 2.12 minutes, purity: 98% (214 nm) (LC-MS method 7).

### Compound 90 3-[3-(23,29-Difluoro-6,10,10-trimethyl-13-oxo-14,25-dioxa-3,4,12,20,31-pentazapentacyclo[24.3.1.12,5.016,24.017,21]hentriaconta-1(30),2,4,16(24),17(21),18,22,26,28-nonaen-6-yl)phenyl]propanoic acid

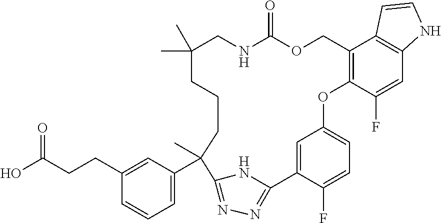 [SMILES: CC1(C)CCCC(C)(c2cccc(CCC(=O)O)c2)c2nnc([nH]2)-c2cc(ccc2F)Oc2c(F)cc3[nH]ccc3c2COC(=O)NC1]

### Compound 91. 23,29-Difluoro-6,10,10-trimethyl-6-phenyl-14,25-dioxa-3,4,12,20,31-pentazapentacyclo[24.3.1.12,5.016,24.017,21]hentriaconta-1(30),2,4,16(24),17(21),18,22,26,28-nonaen-13-one

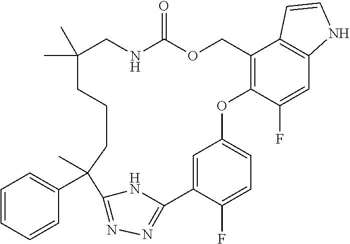 [SMILES: CC1(C)CCCC(C)(c2ccccc2)c2nnc([nH]2)-c2cc(ccc2F)Oc2c(F)cc3[nH]ccc3c2COC(=O)NC1]

Step B: Exchanging (E)-3-(6-Fluoro-5-(4-fluoro-3-(5-(2-(2-fluoro-3-((E)-3-methoxy-3-oxoprop-1-en-1-yl)phenyl)-6-hydroxyhexan-2-yl)-1H-imidazol-2-yl)phenoxy)-1H-indol-4-yl)acrylic acid (Step B product of Example 80) with 7-(3-Bromophenyl)-7-(5-(2-fluoro-5-((6-fluoro-4-(hydroxymethyl)-1H-indol-5-yl)oxy)phenyl)-4H-1,2,4-triazol-3-yl)-3,3-dimethyloctanoic acid (Step A product of this example, 425 mg, 0.637 mmol), the reaction procedure sequence (Steps C, D, E, F, and G) described for Example 80) was used to prepare the title compounds. 90 (26 mg) was obtained as a white solid. 91 (24 mg), the de-bromination product of Heck coupling, corresponding Step C, was not separated until the last step, corresponding Step G of Example 80.

Compound 91: MS (ESI): 586 m/z [M+H]⁺, retention time: 1.83 minutes, purity: >99% (214 nm) (LC-MS method 16). ¹H NMR (400 MHz, CD₃OD): 1H NMR (400 MHz, CD₃OD) δ 7.56-7.05 (m, 10H), 6.73 (s, 1H), 5.55-5.44 (m, 1H), 5.32-5.22 (m, 1H), 2.84-2.72 (m, 2H), 2.22-2.13 (m, 1H), 1.87-1.97 (m, 1H), 1.67-1.74 (m, 2H), 1.31 (s, 3H), 1.10-1.00 (m, 2H), 0.83-0.72 (m, 6H) ppm.

Compound 90: MS (ESI): 658 m/z [M+H]⁺, retention time: 1.43 minutes, purity: >99% (214 nm) (LC-MS method 16). ¹H NMR (400 MHz, CD₃OD): δ 7.46 (s, 1H), 7.40-6.87 (m, 8H), 6.73 (s, 1H), 5.49 (d, J=11.2 Hz, 1H), 5.27 (d, J=11.2 Hz, 1H), 2.89-2.68 (m, 4H), 2.33-2.43 (m, 2H), 2.10-2.20 (m, 1H), 1.87-1.97 (m, 1H), 1.69 (s, 3H), 1.27-1.38 (m, 2H), 0.90-1.18 (m, 2H), 0.80-0.73 (m, 6H) ppm.

### Example 92. Compound 92. 3-[3-(23,29-Difluoro-6,9,9-trimethyl-12-oxo-13,25-dioxa-3,11,20,31-tetrazapentacyclo[24.3.1.12,5.016,24.017,21]hentriaconta-1(30),2,4,16(24),17(21),18,22,26,28-nonaen-6-yl)phenyl]propanoic acid

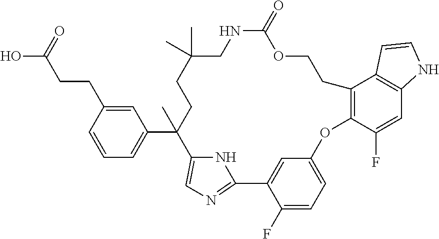 [SMILES: CC1(C)CCC(C)(c2cccc(CCC(=O)O)c2)c2cnc([nH]2)-c2cc(ccc2F)Oc2c(F)cc3[nH]ccc3c2CCOC(=O)NC1]

Exchanging ethyl (E)-3-(5-(3-carbamimidoyl-4-fluorophenoxy)-6-fluoro-1H-indol-4-yl)acrylate (Intermediate 5-2) with 2-fluoro-5-((6-fluoro-4-(2-hydroxyethyl)-1H-indol-5-yl)oxy)benzimidamide (intermediate 5-12, 320 mg, 0.96 mmol) and 7-bromo-5-(3-bromo-2-fluorophenyl)-5-methyl-6-oxoheptyl acetate (Intermediate 1-17) with methyl 8-bromo-6-(3-bromophenyl)-3,3,6-trimethyl-7-oxooctanoate (intermediate 11-8, 346 mg, 0.77 mmol), the reaction procedure sequence (Steps A to G) described for Example 80 was used to prepare the title compound (10 mg) as a white solid. MS (ESI): 657 m/z [M+H]⁺, retention time: 1.48 minutes, purity: >99% (214 nm) (LC-MS method 16). ¹H NMR (400 MHz, CD₃OD): δ 7.42 (s, 1H), 7.31-6.93 (m, 9H), 6.55 (d, J=2.9 Hz, 1H), 4.30-4.37 (m, 2H), 2.84-2.92 (m, 1H), 2.79 (t, J=8.2 Hz, 2H), 2.60-2.69 (m, 1H), 2.49 (t, J=8.2 Hz, 2H), 2.17-2.31 (m, 2H), 1.72-1.83 (m, 1H), 1.59-1.67 (m, 1H), 1.55 (s, 3H), 1.07-1.20 (m, 2H), 0.81 (s, 3H), 0.73 (s, 3H) ppm.

### Example 93. Compound 93A and Compound 93B, Enantiomers 1 and 2 of 3-[3-(23,29-difluoro-6,11,11-trimethyl-13-oxo-14,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

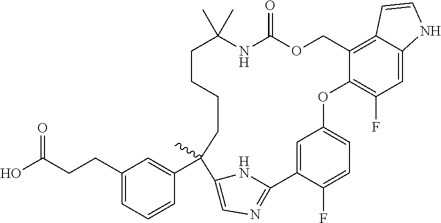 [SMILES: CC1(C)CCCCC(C)(c2cccc(CCC(=O)O)c2)c2cnc([nH]2)-c2cc(ccc2F)Oc2c(F)cc3[nH]ccc3c2COC(=O)N1]

### Example 94. Compound 94. 23,29-Difluoro-6,11,11-trimethyl-6-phenyl-14,25-dioxa-3,12,20,31-tetrazapentacyclo[24.3.1.12,5.016,24.017,21]hentriaconta-1(30),2,4,16,18,21,23,26,28-nonaen-13-one

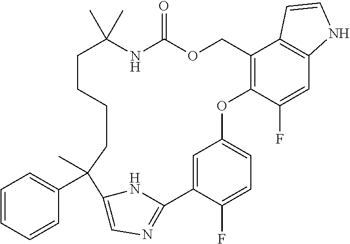 [SMILES: CC1(C)CCCCC(C)(c2ccccc2)c2cnc([nH]2)-c2cc(ccc2F)Oc2c(F)cc3[nH]ccc3c2COC(=O)N1]

### Methyl 7-(3-bromophenyl)-7-(2-(2-fluoro-5-((6-fluoro-4-vinyl-1H-indol-5-yl)oxy)phenyl)-1H-imidazol-5-yl)-2,2-dimethyloctanoate

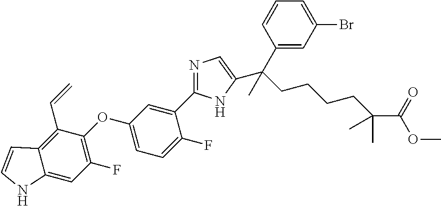 [SMILES: C=Cc1c(Oc2ccc(F)c(-c3ncc(C(C)(CCCCC(C)(C)C(=O)OC)c4cccc(Br)c4)[nH]3)c2)c(F)cc2[nH]ccc12]

Step A: To a stirred solution of 2-fluoro-5-((6-fluoro-4-vinyl-1H-indol-5-yl)oxy)benzimidamide (Intermediate 5-3, 23.3 g, 74.4 mmol) in N,N-dimethylformamide (200 mL) was added sodium bicarbonate (12.5 g, 149 mmol) and methyl 9-bromo-7-(3-bromophenyl)-2,2,7-trimethyl-8-oxononanoate (Intermediate 1-42, 34.4 g, 74.4 mmol). The mixture was stirred at 75° C. for 16 hours, cooled to room temperature and quenched with water (300 mL). The solution was extracted with ethyl acetate (3×150 mL). The combined organic extracts were washed with saturated lithium chloride, dried over sodium sulfate, and concentrated. The residue was purified by flash chromatography to afford the title compound (35.1 g, 69%). MS (ESI): 676, 678 m/z [M+H]⁺, retention time: 2.04 minutes, purity: 90% (254 nm) (LC-MS method 4).

### Methyl 7-(3-bromophenyl)-7-(2-(2-fluoro-5-((6-fluoro-4-formyl-1H-indol-5-yl)oxy)phenyl)-1H-imidazol-5-yl)-2,2-dimethyloctanoate

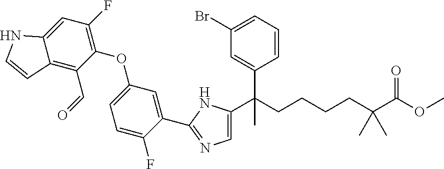 [SMILES: COC(=O)C(C)(C)CCCCC(C)(c1cccc(Br)c1)c1cnc(-c2cc(Oc3c(F)cc4[nH]ccc4c3C=O)ccc2F)[nH]1]

Step B: To a stirred solution of Step A product (35.1 g, 52 mmol) in 500 mL of tetrahydrofuran and 150 mL of water was added sodium periodate (33.3 g, 156 mmol) and osmium tetroxide (0.08 M, 26 mL). The mixture was stirred at room temperature for 16 hours, quenched with water (500 mL), and extracted with ethyl acetate (3×150 mL). The combined organic phases were washed with saturated lithium chloride (2×100 mL), brine (100 mL), dried over sodium sulfate, and concentrated to give the crude title compound (35 g, crude) as an oil. MS (ESI): 678, 680 m/z [M+H]⁺, retention time: 1.92 minutes, purity: 90% (254 nm) (LC-MS method 4).

### Methyl 7-(3-bromophenyl)-7-(2-(2-fluoro-5-((6-fluoro-4-(hydroxymethyl)-1H-indol-5-yl)oxy)phenyl)-1H-imidazol-5-yl)-2,2-dimethyloctanoate

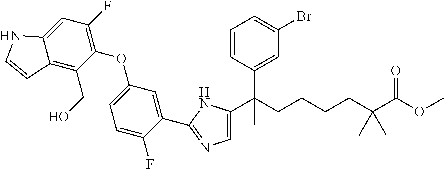 [SMILES: COC(=O)C(C)(C)CCCCC(C)(c1cccc(Br)c1)c1cnc(-c2cc(Oc3c(F)cc4[nH]ccc4c3CO)ccc2F)[nH]1]

Step C: To a stirred solution of Step B product (35 g, crude) in 300 mL of tetrahydrofuran was added sodium borohydride (3.9 g, 103 mmol) and 5 drops of methanol. The mixture was stirred at room temperature for 16 hours, then quenched with water (200 mL), and extracted with ethyl acetate (3×150 mL). The combined organic extracts were washed with brine (10 mL), dried over sodium sulfate, and concentrated. The residue was purified by silica gel column chromatography to afford the title compound (17 g, 45% for 2 steps). MS (ESI): 680, 682 m/z [M+H]⁺, retention time: 1.80 minutes, purity: 95% (254 nm) (LC-MS method 4).

### Compound 93A, 93A, Enantiomers 1 and 2 of 3-[3-(23,29-difluoro-6,11,11-trimethyl-13-oxo-14,25-dioxa-3,12,20,31-tetrazapentacyclo[24.3.1.12,5.016,24.017,21]-hentriaconta-1(30),2,4,16,18,21,23,26,28-nonaen-6-yl)phenyl]propanoic acid

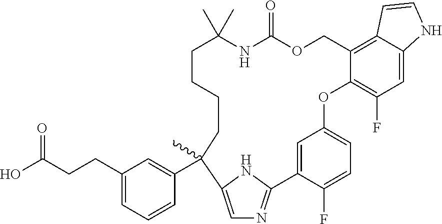 [SMILES: CC1(C)CCCCC(C)(c2cccc(CCC(=O)O)c2)c2cnc([nH]2)-c2cc(ccc2F)Oc2c(F)cc3[nH]ccc3c2COC(=O)N1]

Compound 94. 23,29-Difluoro-6,11,11-trimethyl-6-phenyl-14,25-dioxa-3,12,20,31-tetrazapentacyclo[24.3.1.12,5.016,24.017,21]hentriaconta-1(30),2,4,16,18,21,23,26,28-nonaen-13-one

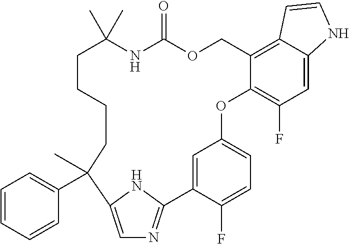 [SMILES: CC1(C)CCCCC(C)(c2ccccc2)c2cnc([nH]2)-c2cc(ccc2F)Oc2c(F)cc3[nH]ccc3c2COC(=O)N1]

Step D: Exchanging (E)-3-(5-(3-(5-(2-(3-Bromo-2-fluorophenyl)-6-hydroxyhexan-2-yl)-1H-imidazol-2-yl)-4-fluorophenoxy)-6-fluoro-1H-indol-4-yl)acrylic acid (Step A product of Example 80) with methyl 7-(3-bromophenyl)-7-(2-(2-fluoro-5-((6-fluoro-4-(hydroxymethyl)-1H-indol-5-yl)oxy)phenyl)-1H-imidazol-5-yl)-2,2-dimethyloctanoate (Step C product of this example, 9.0 g, 13.2 mmol), the reaction procedure sequence (Steps B, C, D, E, F and G, in this order) described for Example 80 was used to prepare the title compounds. 94 (69.4 mg), originated from de-bromination of corresponding Step C of Example 80, was separated at the last step, the corresponding Step G, after the hydrolysis. Another product from corresponding Step G, the racemic acid (230 mg), was subjected to chiral SFC separation under the following condition: Instrument: SFC-80 (Thar, Waters); Column: RRWHELK 20*250 mm, 10 μm; Column temperature: 35° C.; Mobile phase: carbon dioxide/ethanol (0.5% methanol ammonia as additive)=50/50; Flow rate: 80 g/minute; Back pressure: 100 bar; Detection wavelength: 280 nm; Cycle time: 5 minutes; Sample solution: 0.45 g dissolved in 20 mL methanol; Injection volume: 1 mL. The first eluent, Enantiomer 1, is designated as 93A (119 mg); The second eluent, Enantiomer 2, is designated as 93B (123 mg).

Compound 94: MS (ESI): 585 m/z [M+H]⁺, retention time: 1.76 minutes, purity: >99% (254 nm) (LC-MS method 4). 6 7.55 (s, 1H), 7.29 (d, J=3.2 Hz, 1H), 7.27-7.19 (m, 5H), 7.17-7.07 (m, 2H), 6.99 (s, 1H), 6.89 (s, 1H), 6.61 (d, J=3.0 Hz, 1H), 5.25 (d, J=11.6 Hz, 1H), 5.06 (d, J=11.4 Hz, 1H), 2.30-2.26 (m, 1H), 2.14-2.10 (m, 1H), 1.71-1.65 (m, 1H), 1.52 (s, 3H), 1.46-1.25 (m, 3H), 1.19-1.12 (m, 6H), 1.01-0.88 (m, 2H) ppm.

Compound 93A: MS (ESI): 657 m/z [M+H]⁺, retention time: 1.47 minutes, purity: >99% (254 nm) (LC-MS method 4). ¹H NMR (400 MHz, CD₃OD) δ 7.51 (s, 1H), 7.30 (d, J=3.2 Hz, 1H), 7.25 (d, J=10.6 Hz, 1H), 7.18-7.12 (m, 3H), 7.10-6.98 (m, 3H), 6.94 (s, 1H), 6.62 (d, J=3.0 Hz, 1H), 5.26 (d, J=11.7 Hz, 1H), 5.06 (d, J=11.7 Hz, 1H), 2.86-2.82 (m, 2H), 2.53-2.49 (m, 2H), 2.26-2.22 (m, 1H), 2.17-2.07 (m, 1H), 1.65-1.60 (m, 1H), 1.52 (s, 3H), 1.42-1.30 (m, 3H), 1.20-1.10 (m, 6H), 0.99-0.87 (m, 2H) ppm.

Compound 93B: MS (ESI): 657 m/z [M+H]⁺, retention time: 1.47 minutes, purity: >99% (254 nm) (LC-MS method 4). ¹HNMR (400 MHz, CD₃OD) δ 7.51 (s, 1H), 7.30 (d, J=3.2 Hz, 1H), 7.25 (d, J=10.6 Hz, 1H), 7.08-7.02 (m, 3H), 7.10-6.98 (m, 3H), 6.94 (s, 1H), 6.62 (d, J=3.0 Hz, 1H), 5.26 (d, J=11.7 Hz, 1H), 5.06 (d, J=11.7 Hz, 1H), 2.86-2.82 (m, 2H), 2.53-2.48 (m, 2H), 2.26-2.22 (m, 1H), 2.18-2.08 (m, 1H), 1.65-1.60 (m, 1H), 1.52 (s, 3H), 1.41-1.32 (m, 3H), 1.20-1.10 (m, 6H), 0.99-0.88 (m, 2H) ppm.

### Example 95. Compound 95A and Compound 95B. Enantiomers 1 and 2 of 3-[3-(23,29-difluoro-10,10-dimethyl-12-oxo-13,25-dioxa-5,11,20,31-tetrazapentacyclo-[24.3.1.12,5.016,24.017,21]hentriaconta-1(30),2(31),3,16(24),17(21),18,22,26,28-nonaen-6-yl)phenyl]propanoic acid

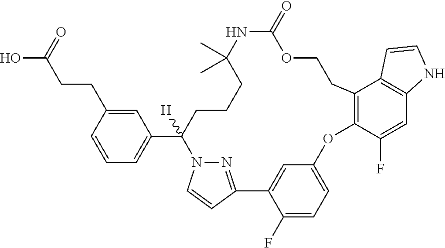 [SMILES: CC1(C)CCCC(c2cccc(CCC(=O)O)c2)n2ccc(n2)-c2cc(ccc2F)Oc2c(F)cc3[nH]ccc3c2CCOC(=O)N1]

### Methyl (E)-6-(3-bromophenyl)-6-(3-(5-((4-(2-ethoxyvinyl)-6-fluoro-1-tosyl-1H-indol-5-yl)oxy)-2-fluorophenyl)-1H-pyrazol-1-yl)-2,2-dimethylhexanoate

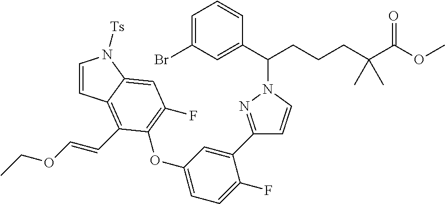 [SMILES: CCO/C=C/c1c(Oc2ccc(F)c(-c3ccn(C(CCCC(C)(C)C(=O)OC)c4cccc(Br)c4)n3)c2)c(F)cc2c1ccn2S(=O)(=O)c1ccc(C)cc1]

Step A: To a stirred and cooled (0° C.) solution of methyl 6-bromo-6-(3-bromophenyl)-2,2-dimethylhexanoate (Intermediate 44-1, 1.32 g, 3.36 mmol) in N,N-dimethylformamide (25 mL) was added (E)-4-(2-ethoxyvinyl)-6-fluoro-5-(4-fluoro-3-(1H-pyrazol-3-yl)phenoxy)-1-tosyl-1H-indole (Intermediate 7A-1, 1.5 g, 1.68 mmol) and cesium carbonate (2.74 g, 8.40 mmol). The mixture was stirred for 4 hours, quenched with 80 mL of water, and extracted with ethyl acetate (3×80 mL). The combined organic phases were washed with brine, dried over sodium sulfate and concentrated. The residue was purified by silica gel column chromatography (eluting with petroleum ether: ethyl acetate=2:1) to give the title compound (1.65 g, 70%) as a yellow liquid. MS (ESI): 846, 848 m/z [M+H]⁺, retention time: 2.70 minutes, purity: 80% (214 nm) (LC-MS method 5).

### Methyl 6-(3-bromophenyl)-6-(3-(2-fluoro-5-((6-fluoro-4-(2-oxoethyl)-1-tosyl-IH-indol-5-yl)oxy)phenyl)-1H-pyrazol-1-yl)-2,2-dimethylhexanoate

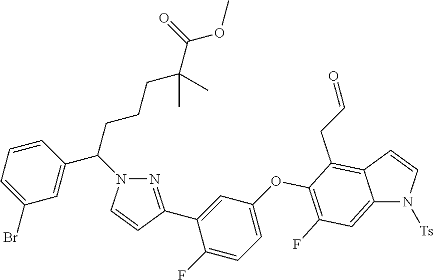 [SMILES: COC(=O)C(C)(C)CCCC(c1cccc(Br)c1)n1ccc(-c2cc(Oc3c(F)cc4c(ccn4S(=O)(=O)c4ccc(C)cc4)c3CC=O)ccc2F)n1]

Step B: To a stirred and cooled (0° C.) solution of Step A product (2.60 g, 3.07 mmol) in tetrahydrofuran (20 mL) was added hydrogen chloride in 1,4-dioxane (4 M, 7.68 mL, 30.7 mmol). The mixture was stirred for 3 hours, then quenched with 50 mL of water, and extracted with ethyl acetate (3×50 mL). The combined organic phases were washed with brine, dried over sodium sulfate, and concentrated to give the crude title compound (2.45 g, crude) as a yellow solid. MS (ESI): 818, 820 m/z [M+H]⁺, retention time: 2.49 minutes, purity: 70% (214 nm) (LC-MS method 5).

### Methyl 6-(3-bromophenyl)-6-(3-(2-fluoro-5-((6-fluoro-4-(2-hydroxyethyl)-1-tosyl-1H-indol-5-yl)oxy)phenyl)-1H-pyrazol-1-yl)-2,2-dimethylhexanoate

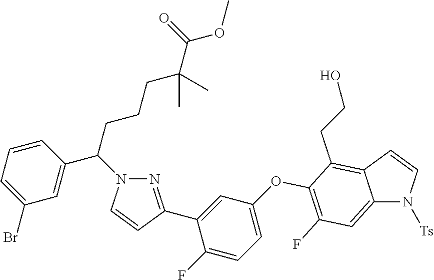 [SMILES: COC(=O)C(C)(C)CCCC(c1cccc(Br)c1)n1ccc(-c2cc(Oc3c(F)cc4c(ccn4S(=O)(=O)c4ccc(C)cc4)c3CCO)ccc2F)n1]

Step C: To a stirred and cooled (0° C.) solution of Step B product (2.45 g, 2.99 mmol) in tetrahydrofuran (30 mL) was added sodium borohydride (113 mg, 2.99 mmol). The mixture was stirred for 1 hour, quenched with 60 mL of water, and extracted with ethyl acetate (3×60 mL). The combined organic phases were washed with brine, dried over sodium sulfate and concentrated. The residue was purified by silica gel column chromatography (eluting with petroleum ether: ethyl acetate=1:1) to give the title compound (1.38 g, 56%) as a yellow solid. MS (ESI): 820, 822 m/z [M+H]⁺, retention time: 2.45 minutes, purity: 90% (214 nm) (LC-MS method 5).

### Compound 95A and 95B. Enantiomers 1 and 2 of 3-[3-(23,29-difluoro-10,10-dimethyl-12-oxo-13,25-dioxa-5,11,20,31-tetrazapentacyclo-[24.3.1.12,5.016,24.017,21]hentriaconta-1(30),2(31),3,16(24),17(21),18,22,26,28-nonaen-6-yl)phenyl]propanoic acid

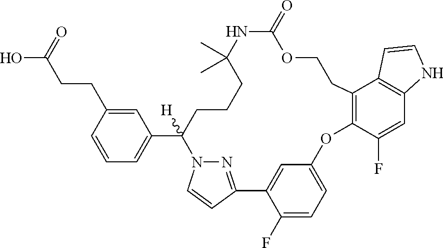 [SMILES: CC1(C)CCCC(c2cccc(CCC(=O)O)c2)n2ccc(n2)-c2cc(ccc2F)Oc2c(F)cc3[nH]ccc3c2CCOC(=O)N1]

Exchanging ethyl (E)-3-(5-(3-(5-(6-acetoxy-2-(3-bromo-2-fluorophenyl)hexan-2-yl)-1H-imidazol-2-yl)-4-fluorophenoxy)-6-fluoro-1H-indol-4-yl)acrylate (Step A product of Example 80) with methyl 6-(3-bromophenyl)-6-(3-(2-fluoro-5-((6-fluoro-4-(2-hydroxyethyl)-1-tosyl-1H-indol-5-yl)oxy)phenyl)-1H-pyrazol-1-yl)-2,2-dimethylhexanoate (Step C product of this example, 1.26 g, 1.53 mmol), the reaction procedure sequence (Steps B, C, D, E, F and G, in this order) described for Example 80 was used to prepare the title compounds. The racemic ethyl ester (195 mg) was subject to chiral SFC separation under the following conditions: Instrument: SFC-80 (Thar, Waters); Column: IC 20*250 mm, 10 μm; Column temperature: 35° C.; Mobile phase: carbon dioxide/methanol (0.2% methyl ethyl amine as additive)=50/50; Flow rate: 80 g/minute; Back pressure: 100 bar; Detection wavelength: 214 nm; Cycle time: 3.3 minutes; Sample solution:200 mg dissolved in 25 mL of methanol; Injection volume: 1.2 mL. The first eluent (81 mg, 42%), Enantiomer 1, was further hydrolyzed to 95A (59.9 mg, 77%, white solid); The second eluent (78 mg, 40%), Enantiomer 2, was further hydrolyzed to 95B (63 mg, 84%), following the conditions described for Step G of Example 80.

Compound 95A: MS (ESI): 643 m/z [M+H]⁺, retention time: 2.17 minutes, purity: 98% (214 nm) (LC-MS method 5). ¹H NMR (400 MHz, CD₃OD) δ 7.84-7.98 (m, 1H), 7.61 (s, 1H), 7.30 (d, J=3.2 Hz, 1H), 7.24-7.21 (m, 3H), 7.18-7.13 (m, 2H), 7.06 (t, J=4.8 Hz, 1H), 6.76-6.73 (m, 1H), 6.67 (s, 1H), 6.55 (d, J=3.2 Hz, 1H), 5.40-5.34 (m, 1H), 4.57-4.53 (m, 1H), 4.03-3.97 (m, 1H), 3.19-3.12 (m, 1H), 2.96-2.89 (m, 1H), 2.89 (t, J=7.6 Hz, 2H), 2.59 (t, J=7.6 Hz, 2H), 1.95-1.89 (m, 2H), 1.43-1.38 (m, 4H), 1.27 (s, 3H), 1.08 (s, 3H) ppm.

Compound 95B: MS (ESI): 643 m/z [M+H]⁺, retention time: 2.17 minutes, purity: >99% (214 nm) (LC-MS method 5). ¹H NMR (400 MHz, CD₃OD) δ 7.85-7.95 (m, 1H), 7.60 (s, 1H), 7.30 (d, J=3.2 Hz, 1H), 7.24-7.21 (m, 3H), 7.18-7.13 (t, J=7.6 Hz, 2H), 7.06 (t, J=4.8 Hz, 1H), 6.76-6.73 (m, 1H), 6.67 (s, 1H), 6.55 (d, J=2.8 Hz, 1H), 5.40-5.34 (d, J=6.0 Hz, 1H), 4.56-4.53 (m, 1H), 4.03-3.97 (m, 1H), 3.19-3.12 (m, 1H), 2.96-2.89 (m, 1H), 2.89 (t, J=7.6 Hz, 2H), 2.59 (t, J=7.6 Hz, 2H), 1.95-1.89 (m, 2H), 1.43-1.38 (m, 4H), 1.26 (s, 3H), 1.08 (s, 3H) ppm.

### Example 96. Compound 96A and Compound 96B. 3-[3-(23,29-difluoro-11,11-dimethyl-13-oxo-8,14,25-trioxa-5,12,20,31-tetrazapentacyclo[24.3.1.12,5.016,24.017,21]hentriaconta-1(30),2(31),3,16,18,21,23,26,28-nonaen-6-yl)phenyl]propanoic acid

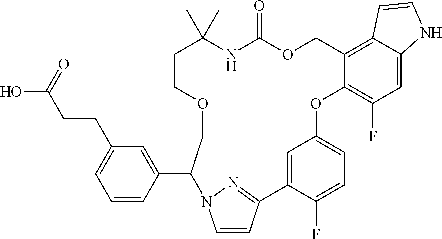 [SMILES: CC1(C)CCOCC(c2cccc(CCC(=O)O)c2)n2ccc(n2)-c2cc(ccc2F)Oc2c(F)cc3[nH]ccc3c2COC(=O)N1]

### Methyl 4-(2-(3-bromophenyl)-2-(3-(2-fluoro-5-((6-fluoro-4-vinyl-1H-indol-5-yl)oxy)phenyl)-1H-pyrazol-1-yl)ethoxy)-2,2-dimethylbutanoate

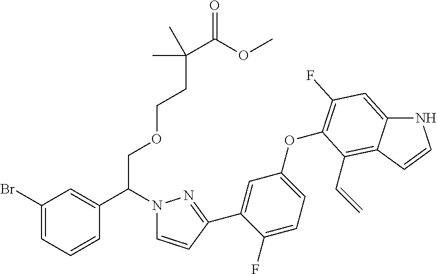 [SMILES: C=Cc1c(Oc2ccc(F)c(-c3ccn(C(COCCC(C)(C)C(=O)OC)c4cccc(Br)c4)n3)c2)c(F)cc2[nH]ccc12]

Step A: To a stirred solution of 6-fluoro-5-(4-fluoro-3-(1H-pyrazol-3-yl)phenoxy)-4-vinyl-1H-indole (Intermediate 9-3, 2.6 g, 7.01 mmol) in N,N-dimethylformamide (50 mL) was added cesium carbonate (4.57 g, 14 mmol) and methyl 4-(2-bromo-2-(3-bromophenyl)ethoxy)-2,2-dimethylbutanoate (Intermediate 63, 2.8 g, 6.31 mmol). The mixture was stirred at room temperature for 18 hours, quenched with water (200 mL), and extracted with ethyl acetate (3×100 mL). The combined organic phases were washed with saturated lithium chloride, brine, dried over sodium sulfate, and concentrated. The residue was purified by automated flash chromatography (eluting with ethyl acetate in petroleum ether from 0 to 25%) to afford the title compound (2.5 g, 46%) as light-yellow solid. MS (ESI): 664, 666 m/z [M+H]⁺, retention time: 2.42 minutes, purity: 88% (214 nm) (LC-MS method 22).

### Methyl 4-(2-(3-bromophenyl)-2-(3-(5-((4-(1,2-dihydroxyethyl)-6-fluoro-1H-indol-5-yl)oxy)-2-fluorophenyl)-1H-pyrazol-1-yl)ethoxy)-2,2-dimethylbutanoate

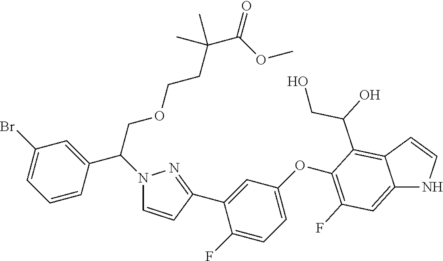 [SMILES: COC(=O)C(C)(C)CCOCC(c1cccc(Br)c1)n1ccc(-c2cc(Oc3c(F)cc4[nH]ccc4c3C(O)CO)ccc2F)n1]

Step B: To a stirred solution of Step A product (2.5 g, 3.27 mmol) in acetone (25 mL) was added N-methylmorpholine N-oxide (0.886 mL, 4.25 mmol, 1.3 eq) and osmium tetroxide (8.32 mg, 0.0327 mmol, in 4.1 mL of water). The mixture was stirred at room temperature overnight, quenched with water (50 mL), and extracted with ethyl acetate (3×50 mL). The combined organic layers were washed with brine, dried over sodium sulfate, and concentrated. The residue was purified by silica gel column chromatography (eluting with ethyl acetate in petroleum ether from 0 to 50%) to give the title compound (1.4 g, 53%) as light-yellow solid. MS (ESI): 698, 700 m/z [M+H]⁺, retention time: 2.14 minutes, purity: 87% (214 nm) (LC-MS method 22).

### Methyl 4-(2-(3-bromophenyl)-2-(3-(2-fluoro-5-((6-fluoro-4-formyl-1H-indol-5-yl)oxy)phenyl)-1H-pyrazol-1-yl)ethoxy)-2,2-dimethylbutanoate

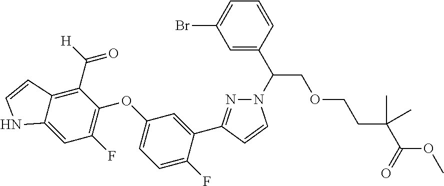 [SMILES: COC(=O)C(C)(C)CCOCC(c1cccc(Br)c1)n1ccc(-c2cc(Oc3c(F)cc4[nH]ccc4c3C=O)ccc2F)n1]

Step C: To a stirred and cooled (0° C.) solution of Step B product (1.4 g, 1.98 mmol) in acetone (50 mL) and water (10 mL) was added sodium periodate (1.27 g, 5.95 mmol) portion wise. The mixture was stirred at room temperature for 15 hours, quenched with 50 mL of water, and extracted with ethyl acetate (3×100 mL). The combined organic phases were washed with brine, dried over sodium sulfate, and concentrated to give crude title compound (1.2 g, crude) as light-yellow solid. This crude compound was used for next step without further purification. MS (ESI): 666, 668 m/z [M+H]⁺, retention time: 2.32 minutes, purity: 99% (214 nm) (LC-MS method 22).

### Methyl 4-(2-(3-bromophenyl)-2-(3-(2-fluoro-5-((6-fluoro-4-(hydroxymethyl)-1H-indol-5-yl)oxy)phenyl)-1H-pyrazol-1-yl)ethoxy)-2,2-dimethylbutanoate

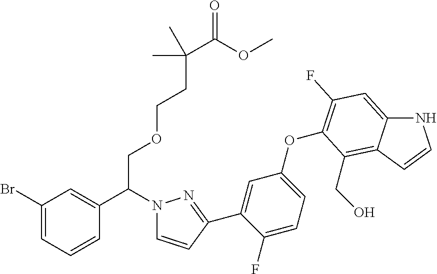 [SMILES: COC(=O)C(C)(C)CCOCC(c1cccc(Br)c1)n1ccc(-c2cc(Oc3c(F)cc4[nH]ccc4c3CO)ccc2F)n1]

Step D: To a stirred and cooled (0° C.) solution of Step C product (1.2 g, 1.78 mmol) in methanol (20 mL) was added sodium borohydride (67.4 mg, 1.78 mmol). The mixture was stirred at room temperature for 2 hours, quenched with 50 mL of water, and extracted with ethyl acetate (3×30 mL). The combined organic phases were washed with brine, dried over sodium sulfate, and concentrated to give crude title compound (1.1 g, crude) as light-yellow solid. This crude compound was used for next step without further purification. MS (ESI): 668, 670 m/z [M+H]⁺, retention time: 2.21 minutes, purity: 98% (214 nm) (LC-MS method 22).

### Compound 96A and 96B. Enantiomers 1 and 2 of 3-[3-(23,29-difluoro-11,11-dimethyl-13-oxo-8,14,25-trioxa-5,12,20,31-tetrazapentacyclo-[24.3.1.12,5.016,24.017,21]hentriaconta-1(30),2(31),3,16,18,21,23,26,28-nonaen-6-yl)phenyl]propanoic acid

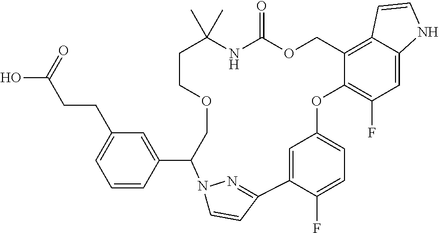 [SMILES: CC1(C)CCOCC(c2cccc(CCC(=O)O)c2)n2ccc(n2)-c2cc(ccc2F)Oc2c(F)cc3[nH]ccc3c2COC(=O)N1]

Step E: Exchanging ethyl (E)-3-(5-(3-(5-(6-acetoxy-2-(3-bromo-2-fluorophenyl)hexan-2-yl)-1H-imidazol-2-yl)-4-fluorophenoxy)-6-fluoro-1H-indol-4-yl)acrylate (Step A product of Example 80) with methyl 4-(2-(3-bromophenyl)-2-(3-(2-fluoro-5-((6-fluoro-4-(hydroxymethyl)-1H-indol-5-yl)oxy)phenyl)-1H-pyrazol-1-yl)ethoxy)-2,2-dimethylbutanoate (Step D product of this example, 1.1 g, 1.61 mmol, crude), the reaction procedure sequence (Steps B, C, D, E, F and G, in this order) described for Example 80 was used to prepare the title compounds. The racemic ethyl ester (120 mg), obtained from corresponding Step F of Example 80, was subjected to chiral prep-HPLC purification. The corresponding analytical separation conditions as follow: Column: OD-H, 250*4.6 mm, 5 μm; Mobile Phase: Hexane (0.1% diethylamine): ethanol (0.1% diethylamine)=80:20. Temperature: 40° C. Flow rate:1.0 mL/minute. Wavelength: 214 nm & 254 nm. Instrument: SHIMADZU. Inject Volume: 8 μL. The first eluent (41 mg, 34%, white solid), Enantiomer 1, was further hydrolyzed to 96A (30 mg, 76%, white solid); The second eluent (46 mg, 38%, white solid), Enantiomer 2, was further hydrolyzed to 96B (22 mg, 50%, white solid), following the conditions described for Step G of Example 80.

Compound 96A: MS (ESI): 645 m/z [M+H]⁺, retention time: 2.17 minutes, purity: 99% (214 nm) (LC-MS method 12). ¹H NMR (400 MHz, DMSO-d₆) δ 12.18 (brs, 1H), 11.39 (s, 1H), 7.85 (d, J=2 Hz, 1H), 7.54 (br s, 1H), 7.54-7.39 (m, 2H), 7.25-7.12 (m, 4H), 7.05-7.03 (m, 1H), 6.99-6.97 (m, 1H), 6.65-6.58 (m, 1H), 6.00 (br s, 1H), 5.62-5.59 (m, 1H), 5.20 (d, J=11.6 Hz, 1H), 5.12 (d, J=11.6 HZ, 1H), 4.16 (t, J=10.8 Hz, 1H), 3.79-3.77 (m, 1H), 3.52-3.49 (m, 1H), 3.28-3.17 (m, 1H), 2.77 (t, J=7.6 Hz, 2H), 2.48 (t, J=7.6 Hz, 2H), 1.72-1.54 (m, 2H), 1.03-1.00 (m, 6H) ppm.

Compound 96B: MS (ESI): 645 m/z [M+H]⁺, retention time: 2.17 minutes, purity: 99% (214 nm) (LC-MS method 12). ¹H NMR (400 MHz, DMSO-d₆) δ 12.07 (brs, 1H), 11.34 (s, 1H), 7.85 (d, J=2 Hz, 1H), 7.54 (br s, 1H), 7.54-7.39 (m, 2H), 7.25-7.12 (m, 4H), 7.05-7.03 (m, 1H), 6.99-6.97 (m, 1H), 6.65-6.58 (m, 2H), 6.02 (s, 1H), 5.63-5.59 (m, 1H), 5.20 (d, J=11.6 Hz, 1H), 5.12 (d, J=11.6 Hz, 1H), 4.17 (t, J=11.2 Hz, 1H), 3.80-3.77 (m, 1H), 3.51-3.47 (m, 1H), 3.27-3.17 (m, 1H), 2.77 (t, J=7.6 Hz, 2H), 2.47 (t, J=7.6 Hz, 2H), 1.71-1.56 (m, 2H), 1.03-1.00 (m, 6H) ppm.

### Example 97. Compound 97A and Compound 97B. Enantiomers 1 and 2 of 3-[3-(23,29-difluoro-3,11,11-trimethyl-13-oxo-14,25-dioxa-3,4,12,20,31-pentazapentacyclo-[24.3.1.12,5.016,24.017,21]hentriaconta-1(30),2(31),4,16,18,21,23,26,28-nonaen-6-yl)phenyl]propanoic acid

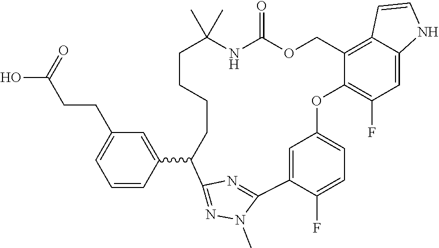 [SMILES: Cn1nc2nc1-c1cc(ccc1F)Oc1c(F)cc3[nH]ccc3c1COC(=O)NC(C)(C)CCCCC2c1cccc(CCC(=O)O)c1]

### Methyl 7-(3-bromophenyl)-7-(5-(2-fluoro-5-((6-fluoro-4-vinyl-1H-indol-5-yl)oxy)phenyl)-1-methyl-1H-1,2,4-triazol-3-yl)-2,2-dimethylheptanoate

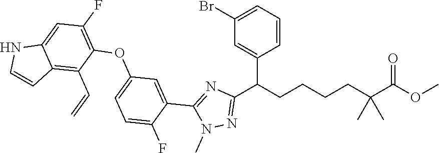 [SMILES: C=Cc1c(Oc2ccc(F)c(-c3nc(C(CCCCC(C)(C)C(=O)OC)c4cccc(Br)c4)nn3C)c2)c(F)cc2[nH]ccc12]

Step A: To a solution of methyl 2-fluoro-5-((6-fluoro-4-vinyl-1H-indol-5-yl)oxy)benzimidothioate hydroiodide (Intermediate 65-1, 3.64 g, 0.00771 mol) and methyl 7-(3-bromophenyl)-2,2-dimethyl-8-(2-methylhydrazineyl)-8-oxooctanoate (Intermediate 67, 2.8 g, 0.00701 mol) in pyridine (60 mL) was heated at 80° C. for 4 hours. The mixture was concentrated. The residue was dissolved in ethyl acetate (100 mL), washed with water, brine, dried over sodium sulfate, and concentrated. The crude product was purified by silica gel column chromatography (petroleum ether/ethyl acetate=2:1) to give the title compound (2.7 g, 57%) as a light-yellow oil. MS (ESI): 677, 679 m/z [M+H]⁺, retention time: 2.41 minutes, purity: 91% (214 nm) (LC-MS method 14).

### Compound 97A and 97B. Enantiomers 1 and 2 of 3-[3-(23,29-difluoro-3,11,11-trimethyl-13-oxo-14,25-dioxa-3,4,12,20,31-pentazapentacyclo-[24.3.1.12,5.016,24.017,21]hentriaconta-1(30),2(31),4,16,18,21,23,26,28-nonaen-6-yl)phenyl]propanoic acid

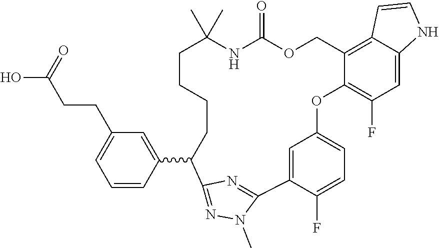 [SMILES: Cn1nc2nc1-c1cc(ccc1F)Oc1c(F)cc3[nH]ccc3c1COC(=O)NC(C)(C)CCCCC2c1cccc(CCC(=O)O)c1]

Exchanging methyl 4-(2-(3-bromophenyl)-2-(3-(2-fluoro-5-((6-fluoro-4-vinyl-1H-indol-5-yl)oxy)phenyl)-1H-pyrazol-1-yl)ethoxy)-2,2-dimethylbutanoate (Step A product of Examples 96A and 96B) with methyl 7-(3-bromophenyl)-7-(5-(2-fluoro-5-((6-fluoro-4-vinyl-1H-indol-5-yl)oxy)phenyl)-1-methyl-1H-1,2,4-triazol-3-yl)-2,2-dimethylheptanoate (Step A product of this example, 3 g, 4.72 mmol), the reaction procedure sequence (Steps B, C, D of Example 96A and 96B, followed by Steps B, C, D, E, F and G of Example 80, in this order) was used to prepare the title compounds. The racemic ethyl ester (350 mg), obtained from corresponding Step F of Example 80, was subjected to chiral SFC separation under the following conditions: Instrument: SFC-80 (Thar, Waters; Column: SSWHELK 20*250 mm, 10 μm; Column temperature: 35° C.; Mobile phase: carbon dioxide/isopropanol (0.2% methanol ammonia as additive)=45/55; Flow rate: 75 g/minute; Back pressure: 100 bar; Detection wavelength: 230 nm; Cycle time: 3 minutes; Sample solution: 350 mg dissolved in 25 mL of methanol; Injection volume: 1 mL. The first eluent (130 mg, 37%), Enantiomer 1, was further hydrolyzed to 97A (86 mg, 68%); The second eluent (130 mg, 37%), Enantiomer 2, was further hydrolyzed to 97B (85 mg, 68%), following the reaction conditions described in Step G of Example 80.

Compound 97A: MS (ESI): 658 m/z [M+H]⁺, retention time: 2.16 minutes, purity: 97% (214 nm) (LC-MS method 24). ¹H NMR (400 MHz, CD₃OD) δ 7.53-7.48 (m, 1H), 7.39-7.33 (m, 2H), 7.29-7.14 (m, 5H), 7.04 (d, J=7.2 Hz, 1H), 6.74 (d, J=2.8 Hz, 1H), 6.21 (s, 1H), 5.39 (s, 2H), 4.05-4.00 (m, 2H), 3.80-3.78 (m, 3H), 2.86 (t, J=7.6 Hz, 3H), 2.55 (t, J=7.6 Hz, 2H), 2.32-2.22 (m, 1H), 1.93-1.84 (m, 1H), 1.42-1.30 (m, 3H), 1.19 (s, 6H), 0.98-0.87 (m, 1H) ppm.

Compound 97B: MS (ESI): 658 m/z [M+H]⁺, retention time: 2.16 minutes, purity: >99% (214 nm) (LC-MS method 24). ¹H NMR (400 MHz, CD₃OD) δ 7.53-7.48 (m, 1H), 7.39-7.33 (m, 2H), 7.29-7.14 (m, 5H), 7.04 (d, J=7.2 Hz, 1H), 6.74 (d, J=2.8 Hz, 1H), 6.21 (s, 1H), 5.39 (s, 2H), 4.05-4.00 (m, 2H), 3.80-3.78 (m, 3H), 2.86 (t, J=7.6 Hz, 3H), 2.55 (t, J=7.6 Hz, 2H), 2.32-2.22 (m, 1H), 1.93-1.84 (m, 1H), 1.42-1.30 (m, 3H), 1.19 (s, 6H), 0.98-0.86 (m, 1H) ppm.

### Example 98. Compound 98A and Compound 98B. Enantiomers 1 and 2 of 3-[3-[(6S)-23,29-difluoro-3,6,9,9-tetramethyl-12-oxo-11,25-dioxa-3,4,13,20,31-pentazapentacyclo-[24.3.1.12,5.016,24.017,21]hentriaconta-1(30),2(31),4,16,18,21,23,26,28-nonaen-6-yl]phenyl]propanoic acid

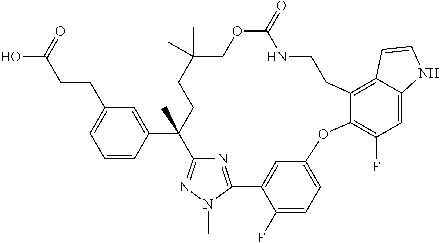 [SMILES: Cn1nc2nc1-c1cc(ccc1F)Oc1c(F)cc3[nH]ccc3c1CCNC(=O)OCC(C)(C)CC[C@@]2(C)c1cccc(CCC(=O)O)c1]

Exchanging benzyl (5-(3-bromophenyl)-2,2,5-trimethyl-6-(2-methylhydrazineyl)-6-oxohexyl)(methyl)carbamate (Intermediate 67-1) with 5-(3-bromophenyl)-2,2,5-trimethyl-6-(2-methylhydrazineyl)-6-oxohexyl 2,2,2-trifluoroacetate (Intermediate 67-4, 1.09 g, 2.4 mmol), the reaction procedure sequence (Step A of Example 75A and 75B, followed by Steps B, C, D, E, F and G of Example 80) was used to prepare the title compounds. The racemic ethyl ester (530 mg), obtained from corresponding Step F of Example 80, was subjected to the chiral Prep-HPLC separation under the following condition: Instrument: Gilson-281; Column: IG 20*250 mm, 10 μm; Mobile Phase: n-Hexane (0.1% diethylamine): ethanol (0.1% diethylamine)=75:25; Flow Rate: 40 mL/minute; Run time per injection: 10 minutes; Injection: 0.4 mL; Sample solution: 490 mg in 14 mL of methanol. The first eluent (160 mg, 33%), Enantiomer 1, was further hydrolyzed to 98A (86 mg, 56%, white solid); The second eluent (160 mg, 33%), Enantiomer 2, was further hydrolyzed to 98B (95 mg, 62%, white solid), following the conditions described in Step G of Example 80.

Compound 98A: MS (ESI): 672 m/z [M+H]⁺, retention time: 2.02 minutes, purity: >99% (214 nm) (LC-MS method 5). ¹H NMR (400 MHz, CD₃OD) δ 7.48-7.03 (m, 9H), 6.63-6.50 (m, 1H), 3.83 (s, 3H), 3.70-3.37 (m, 4H), 3.11 (t, J=5.6 Hz, 2H), 2.86 (t, J=7.2 Hz, 2H), 2.55 (t, J=7.2 Hz, 2H), 2.33-2.25 (m, 1H), 2.00-1.90 (m, 1H), 1.66 (s, 3H), 1.16-1.11 (m, 2H), 0.85-0.69 (m, 5H), 0.60-0.50 (m, 1H) ppm.

Compound 98B: MS (ESI): 672 m/z [M+H]⁺, retention time: 2.02 minutes, purity: >99% (214 nm) (LC-MS method 5). ¹H NMR (400 MHz, CD₃OD) δ 7.49-7.03 (m, 9H), 6.73-6.52 (m, 1H), 3.83 (s, 3H), 3.70-3.37 (m, 4H), 3.11 (t, J=5.6 Hz, 2H), 2.87 (t, J=7.2 Hz, 2H), 2.55 (t, J=7.2 Hz, 2H), 2.32-2.23 (m, 1H), 2.01-1.89 (m, 1H), 1.66 (s, 3H), 1.16-1.12 (m, 2H), 0.85-0.69 (d, J=22.0 Hz, 5H), 0.60-0.51 (m, 1H) ppm.

### Example 99. Compound 99A and Compound 99B. Enantiomers 1 and 2 of 3-[3-(22,28-difluoro-3,6,10,10-tetramethyl-12-oxo-13,24-dioxa-3,4,11,19,30-pentazapentacyclo-[23.3.1.12,5.015,23.016,20]-triaconta-1(29),2(30),4,15,17,20,22,25,27-nonaen-6-yl)phenyl]propanoic acid

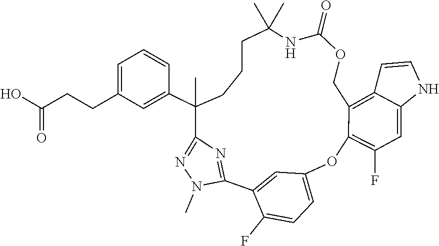 [SMILES: Cn1nc2nc1-c1cc(ccc1F)Oc1c(F)cc3[nH]ccc3c1COC(=O)NC(C)(C)CCCC2(C)c1cccc(CCC(=O)O)c1]

Exchanging benzyl (5-(3-bromophenyl)-2,2,5-trimethyl-6-(2-methylhydrazineyl)-6-oxohexyl)(methyl)carbamate (Intermediate 67-1) with methyl 6-(3-bromophenyl)-2,2,6-trimethyl-7-(2-methylhydrazineyl)-7-oxoheptanoate (Intermediate 67-5, 2.5 g, 6.26 mmol), and ethyl 3-(6-fluoro-5-(4-fluoro-3-(imino(methylthio)methyl)phenoxy)-1H-indol-4-yl)propanoate hydroiodide (Intermediate 65) with methyl 2-fluoro-5-((6-fluoro-4-vinyl-1H-indol-5-yl)oxy)benzimidothioate (Intermediate 65-1, 2.16 g, 6.26 mmol), the reaction procedure sequence (Step A of Example 75A and 75B, followed by Steps B, C, D of Example 96A and 96B, and Steps B, C, D, E, F and G of Example 80, in this order) was used to prepare the title compounds. The racemic ethyl ester (197 mg), obtained from corresponding Step F of Example 80, was subjected to the chiral Prep-HPLC separation under the following condition: Instrument: Gilson-281; Column: OZ 20*250 mm, 10 μm; Mobile Phase: n-Hexane (0.1% diethylamine): ethanol (0.1% diethylamine)=90:10; Flow Rate: 40 mL/minute; Run time per injection: 15 minutes; Injection: 0.6 mL; Sample solution: 197 mg in 10 mL of methanol. The first eluent (36 mg, 18%), Enantiomer 1, was further hydrolyzed to 99A (34 mg, 98%, white solid); The second eluent (85 mg, 43%), Enantiomer 2, was further hydrolyzed to 99B (79 mg, 94%, white solid), following the conditions described in Step G of Example 80.

Compound 99A: MS (ESI): 658 m/z [M+H]⁺, retention time: 1.92 minutes, purity: 98% (214 nm) (LC-MS method 25). ¹H NMR (400 MHz, CD₃OD) δ 7.60-7.56 (m, 1H), 7.38 (t, J=9.0 Hz, 1H), 7.32 (d, J=3.2 Hz, 1H), 7.25 (d, J=10.8 Hz, 1H), 7.14 (t, J=7.6 Hz, 1H), 7.05-6.96 (m, 4H), 6.75 (d, J=3.2 Hz, 1H), 5.46 (d, J=11.2 Hz, 1H), 5.30 (d, J=11.2 Hz, 1H), 3.70 (s, 3H), 2.81-2.74 (m, 2H), 2.46 (t, J=7.6 Hz, 2H), 2.25-2.15 (m, 1H), 1.86-1.80 (m, 1H), 1.67 (s, 3H), 1.56-1.38 (m, 4H), 1.29 (s, 3H), 1.15 (s, 3H) ppm.

Compound 99A: MS (ESI): 658 m/z [M+H]⁺, retention time: 1.92 minutes, purity: 98% (214 nm) (LC-MS method 25). ¹H NMR (400 MHz, CD₃OD) δ 7.60-7.55 (m, 1H), 7.38 (t, J=8.8 Hz, 1H), 7.33 (d, J=3.2 Hz, 1H), 7.25 (d, J=10.4 Hz, 1H), 7.14 (t, J=7.6 Hz, 1H), 7.05-6.96 (m, 4H), 6.75 (d, J=3.2 Hz, 1H), 5.46 (d, J=11.2 Hz, 1H), 5.30 (d, J=11.2 Hz, 1H), 3.70 (s, 3H), 2.81-2.74 (m, 2H), 2.46 (t, J=7.6 Hz, 2H), 2.25-2.19 (m, 1H), 1.86-1.80 (m, 1H), 1.67 (s, 3H), 1.56-1.40 (m, 4H), 1.29 (s, 3H), 1.15 (s, 3H) ppm.

### Example 100. Compound 100A and Compound 100B. Enantiomers 1 and 2 of 3-[3-(23,29-difluoro-3,6,11,11-tetramethyl-13-oxo-9,14,25-trioxa-3,4,12,20,31-pentazapentacyclo-[24.3.1.12,5.016,24.017,21]hentriaconta-1(30),2(31),4,16,18,21,23,26,28-nonaen-6-yl)phenyl]propanoic acid

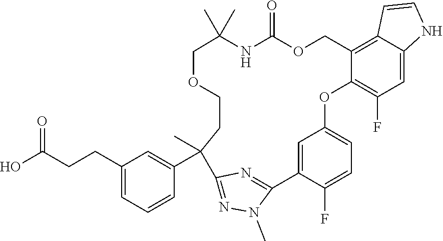 [SMILES: Cn1nc2nc1-c1cc(ccc1F)Oc1c(F)cc3[nH]ccc3c1COC(=O)NC(C)(C)COCCC2(C)c1cccc(CCC(=O)O)c1]

Exchanging benzyl (5-(3-bromophenyl)-2,2,5-trimethyl-6-(2-methylhydrazineyl)-6-oxohexyl)(methyl)carbamate (Intermediate 67-1) with methyl 3-(3-(3-bromophenyl)-3-methyl-4-(2-methylhydrazineyl)-4-oxobutoxy)-2,2-dimethylpropanoate (Intermediate 67-6, 6 g, 14.4 mmol), and ethyl 3-(6-fluoro-5-(4-fluoro-3-(imino(methylthio)methyl)phenoxy)-1H-indol-4-yl)propanoate hydroiodide (Intermediate 65) with methyl 2-fluoro-5-((6-fluoro-4-vinyl-1H-indol-5-yl)oxy)benzimidothioate hydroiodide (Intermediate 65-1, 4.98 g, 14.4 mmol), the reaction procedure sequence (Step A of Example 75A and 75B, followed by Steps B, C, D of Example 96A and 96B, and Steps B, C, D, E, F and G of Example 80, in this order) was used to prepare the title compounds. The racemic ethyl ester (300 mg), obtained from corresponding Step F of Example 80, was subjected to chiral Prep-HPLC separation under the following condition: Instrument: Gilson-281; Column: OD 20*250 mm, 10 μm; Mobile Phase: n-Hexane (0.1% diethylamine): ethanol (0.1% diethylamine)=80:20; Flow Rate: 50 mL/minute; Run time per injection: 10 minutes; Injection: 1.0 mL; Sample solution: 300 mg in 15 mL of methanol. The first eluent (75 mg, 25%), Enantiomer 1, was further hydrolyzed to 100A (38.6 mg, 54%, white solid); The second eluent (75 mg, 25%), Enantiomer 2, was further hydrolyzed to 100B (35 mg, 48%, white solid), following the conditions described in Step G of Example 80.

Compound 100A: MS (ESI): 674 m/z [M+H]⁺, retention time: 2.09 minutes, purity: 85% (214 nm) (LC-MS method 4). ¹H NMR (400 MHz, CD₃OD) δ 7.53-7.44 (m, 1H), 7.38-7.23 (m, 3H), 7.10-6.99 (m, 5H), 6.65 (d, J=3.2 Hz, 1H), 5.35 (d, J=10.8 Hz, 1H), 5.30-5.25 (m, 1H), 3.77 (s, 3H), 3.37 (t, J=1.6 Hz, 2H), 3.04-2.95 (m, 2H), 2.79-2.75 (m, 2H), 2.72-2.59 (m, 1H), 2.47 (t, J=7.6 Hz, 2H), 2.24-2.12 (m, 1H), 1.68 (s, 3H), 1.14 (s, 3H), 1.08 (s, 3H) ppm.

Compound 100B: MS (ESI): 674 m/z [M+H]⁺, retention time: 2.09 minutes, purity: 85% (254 nm) (LC-MS method 4). ¹H NMR (400 MHz, CD₃OD) δ 7.53-7.44 (m, 1H), 7.38-7.23 (m, 3H), 7.10-6.99 (m, 5H), 6.65 (d, J=3.2 Hz, 1H), 5.35 (d, J=10.8 Hz, 1H), 5.30-5.25 (m, 1H), 3.77 (s, 3H), 3.37 (t, J=1.6 Hz, 2H), 3.05-2.94 (m, 2H), 2.79-2.75 (m, 2H), 2.72-2.59 (m, 1H), 2.47 (t, J=7.6 Hz, 2H), 2.24-2.12 (m, 1H), 1.68 (s, 3H), 1.14 (s, 3H), 1.08 (s, 3H) ppm.

### Example 101. Compound 101A and Compound 101B. Diastereomers 1 and 2 of 3-[3-(23,29-difluoro-3,6,11,11-tetramethyl-13-oxo-14,25-dioxa-3,4,12,20,31-pentazapentacyclo[24.3.1.12,5.016,24.017,21]-hentriaconta-1(30),2(31),4,16,18,21,23,26,28-nonaen-6-yl)phenyl]-2-methyl-propanoic acid

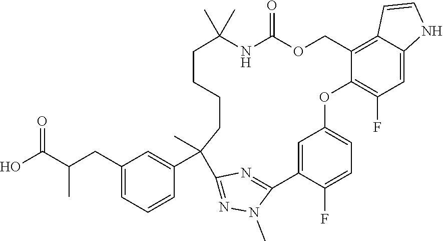 [SMILES: CC(Cc1cccc(C2(C)CCCCC(C)(C)NC(=O)OCc3c(c(F)cc4[nH]ccc34)Oc3ccc(F)c(c3)-c3nc2nn3C)c1)C(=O)O]

### Example 102. Compound 102.23,29-difluoro-3,6,11,11-tetramethyl-6-phenyl-14,25-dioxa-3,4,12,20,31-pentazapentacyclo[24.3.1.12,5.016,24.017,21]hentriaconta-1(30),2(31),4,16,18,21,23,26,28-nonaen-13-one

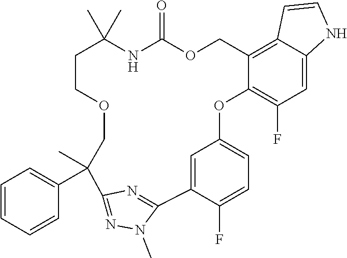 [SMILES: Cn1nc2nc1-c1cc(ccc1F)Oc1c(F)cc3[nH]ccc3c1COC(=O)NC(C)(C)CCOCC2(C)c1ccccc1]

### 6-(3-Bromophenyl)-23,29-difluoro-3,6,11,11-tetramethyl-14,25-dioxa-3,4,12,20,31-pentazapentacyclo[24.3.1.12,5.016,24.017,21]hentriaconta-1(30),2(31),4,16,18,21,23,26,28-nonaen-13-one

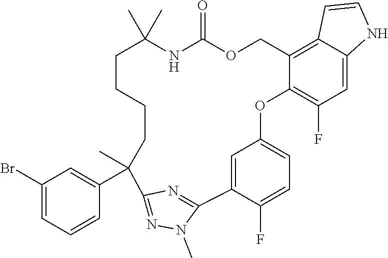 [SMILES: Cn1nc2nc1-c1cc(ccc1F)Oc1c(F)cc3[nH]ccc3c1COC(=O)NC(C)(C)CCCCC2(C)c1cccc(Br)c1]

Step A: Exchanging benzyl (5-(3-bromophenyl)-2,2,5-trimethyl-6-(2-methylhydrazineyl)-6-oxohexyl)(methyl)carbamate (Intermediate 67-1) with methyl 7-(3-bromophenyl)-2,2,7-trimethyl-8-(2-methylhydrazineyl)-8-oxooctanoate (Intermediate 67-7, 2.80 g, 6.77 mmol), and ethyl 3-(6-fluoro-5-(4-fluoro-3-(imino(methylthio)methyl)phenoxy)-1H-indol-4-yl)propanoate hydroiodide (Intermediate 65) with methyl 2-fluoro-5-((6-fluoro-4-vinyl-1H-indol-5-yl)oxy)benzimidothioate hydroiodide (Intermediate 65-1, 3.52 g, 7.45 mmol), the reaction procedure sequence (Step A of Example 75A and 75B, followed by Steps B, C, D of Example 96A and 96B, and Steps B, E, F of Example 80, in this order) was used to prepare the title compounds (300 mg). MS (ESI): 678, 680 m/z [M+H]⁺, retention time: 2.18 minutes, purity: 98% (214 nm) (LC-MS method 25).

### Ethyl (E)-3-[3-(23,29-difluoro-3,6,11,11-tetramethyl-13-oxo-14,25-dioxa-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-prop-2-enoate

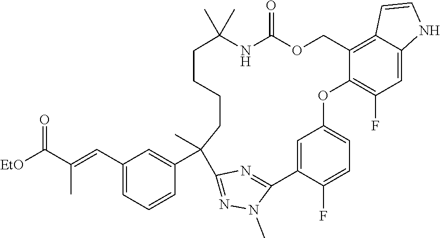 [SMILES: CCOC(=O)/C(C)=C/c1cccc(C2(C)CCCCC(C)(C)NC(=O)OCc3c(c(F)cc4[nH]ccc34)Oc3ccc(F)c(c3)-c3nc2nn3C)c1]

### Compound 102: 23,29-Difluoro-3,6,11,11-tetramethyl-6-phenyl-14,25-dioxa-3,4,12,20,31-pentazapentacyclo[24.3.1.12,5.016,24.017,21]hentriaconta-1(30),2(31),4,16,18,21,23,26,28-nonaen-13-one

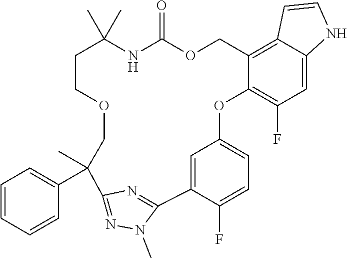 [SMILES: Cn1nc2nc1-c1cc(ccc1F)Oc1c(F)cc3[nH]ccc3c1COC(=O)NC(C)(C)CCOCC2(C)c1ccccc1]

Step B: To a stirred and degassed solution of Step A product (300 mg, 0.442 mmol) in 1,4-dioxane (20 mL) and water (4 mL) was added ethyl (E)-2-methyl-3-(4,4,5,5-tetramethyl-1,3,2-dioxaborolan-2-yl)acrylate (159 mg, 0.663 mmol), 1,1′-bis(diphenylphosphino)ferrocene-palladium(II)dichloride dichloromethane complex (18.1 mg, 0.0221 mmol) and cesium carbonate (288 mg, 0.884 mmol). The mixture was stirred at 100° C. overnight, cooled to room temperature, and quenched with 30 mL of water. The solution was extracted with ethyl acetate (3×30 mL). The combined organic phases were washed with brine, dried over sodium sulfate, and concentrated. The residue was purified by Prep-TLC (developing with dichloromethane:methanol=60:1) to give the de-bromination product 102 (23 mg, 9%, white solid) and ethyl (E)-3-[3-(23,29-difluoro-3,6,11,11-tetramethyl-13-oxo-14,25-dioxa-3,4,12,20,31-pentazapentacyclo [24.3.1.12,5.016,24.017,21]hentriaconta-1(30),2(31),4,16,18,21,23,26,28-nonaen-6-yl)phenyl]-2-methyl-prop-2-enoate (250 mg, 79%, white solid).

Compound 102: MS (ESI): 600 m/z [M+H]⁺, retention time: 2.09 minutes, purity: >99% (214 nm) (LC-MS method 25). ¹H NMR (400 MHz, DMSO-d₆) δ 11.35 (s, 1H), 7.44-7.36 (m, 3H), 7.257.22 (m, 4H), 7.17-7.14 (m, 3H), 6.71-6.68 (m, 2H), 5.22 (d, J=12.4 Hz, 1H), 5.13 (d, J=12.4 Hz, 1H), 3.79 (s, 3H), 2.32-2.29 (m, 1H), 2.02-1.78 (m, 3H), 1.60 (s, 3H), 1.42-1.34 (m, 2H), 1.12 (s, 3H), 1.05 (s, 3H), 0.91-0.77 (m, 2H) ppm.

Ethyl (E)-3-[3-(23,29-difluoro-3,6,11,11-tetramethyl-13-oxo-14,25-dioxa-3,4,12,20,31-pentazapentacyclo [24.3.1.12,5.016,24.017,21]hentriaconta-1(30),2(31),4,16,18,21,23,26,28-nonaen-6-yl)phenyl]-2-methyl-prop-2-enoate: MS (ESI): 712 m/z [M+H]⁺, retention time: 2.20 minutes, purity: 92% (214 nm) (LC-MS method 25).

### Ethyl 3-[3-(23,29-difluoro-3,6,11,11-tetramethyl-13-oxo-14,25-dioxa-3,4,12,20,31-pentazapentacyclo[24.3.1.12,5.016,24.017,21]-hentriaconta-1(30),2(31),4,16,18,21,23,26,28-nonaen-6-yl)phenyl]-2-methyl-propanoate

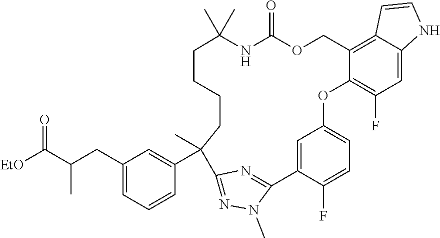 [SMILES: CCOC(=O)C(C)Cc1cccc(C2(C)CCCCC(C)(C)NC(=O)OCc3c(c(F)cc4[nH]ccc34)Oc3ccc(F)c(c3)-c3nc2nn3C)c1]

Step C: To a stirred solution of ethyl (E)-3-[3-(23,29-difluoro-3,6,11,11-tetramethyl-13-oxo-14,25-dioxa-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-prop-2-enoate (Step B product, 250 mg, 0.351 mmol) in methanol (10 mL) was added palladium on carbon (10%, 37.4 mg, 50% wet). The mixture was stirred at room temperature for 2 hours under hydrogen balloon and filtered through a pad of Celite. The filtrate was concentrated to give the title compound (200 mg, 80%) as a white solid.

### Example 101A and 101B. Diastereomers 1 and 2 of 3-[3-(23,29-difluoro-3,6,11,11-tetramethyl-13-oxo-14,25-dioxa-3,4,12,20,31-pentazapentacyclo[24.3.1.12,5.016,24.017,21]-hentriaconta-1(30),2(31),4,16,18,21,23,26,28-nonaen-6-yl)phenyl]-2-methyl-propanoic acid

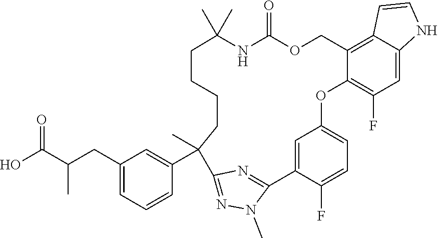 [SMILES: CC(Cc1cccc(C2(C)CCCCC(C)(C)NC(=O)OCc3c(c(F)cc4[nH]ccc34)Oc3ccc(F)c(c3)-c3nc2nn3C)c1)C(=O)O]

Step D: The Step C product (379 mg, 0.53 mmol) was subjected to chiral Prep-HPLC separation under the following condition: Instrument: YMC, K-Pre LAB 100S; Column: CHIRALCEL OZ (OZ-5 cm-10 μm-400 g); Column temperature: 38° C.; Mobile phase: Hexane/isopropanol=80/20 (V/V); Flow rate: 60 mL/minute; Detection wavelength: 214 nm; Cycle time: 310s; Sample solution: 379 mg dissolved in 90 mL methanol. Injection volume: 3 mL (loading: 12.6 mg/injection). The first eluent (150 mg, 38%), Enantiomer 1, was further hydrolyzed to 101A (109 mg, 76%, white solid); The second eluent (180 mg, 45%), Enantiomer 2, was further hydrolyzed to 101B (115 mg, 66%, white solid), following the conditions described in Step G of Example 1.

Compound 101A: MS (ESI): 686 m/z [M+H]⁺, retention time: 1.96 minutes, purity: >99% (214 nm) (LC-MS method 25). ¹H NMR (400 MHz, CD₃OD) δ 7.34-7.30 (m, 3H), 7.29-7.22 (m, 2H), 7.16 (t, J=7.6 Hz, 1H), 7.09-7.04 (m, 2H), 7.01 (d, J=7.6 Hz, 1H), 6.70 (d, J=2.8 Hz, 1H), 6.27 (s, 1H), 5.36-5.26 (m, 2H), 3.82 (d, J=2.0 Hz, 3H), 2.98-2.93 (m, 1H), 2.62-2.54 (m, 2H), 2.34-2.29 (m, 1H), 2.08-2.05 (m, 1H), 1.64 (s, 3H), 1.61-1.55 (m, 1H), 1.48-1.41 (m, 2H), 1.36-1.31 (m, 1H), 1.19-1.17 (m, 6H), 1.10-1.06 (m, 3H), 0.95-0.90 (m, 2H) ppm.

Compound 101A: MS (ESI): 686 m/z [M+H]⁺, retention time: 1.96 minutes, purity: >99% (214 nm) (LC-MS method 25). ¹H NMR (400 MHz, CD₃OD) δ 7.34-7.30 (m, 3H), 7.29-7.22 (m, 2H), 7.16 (t, J=7.6 Hz, 1H), 7.09-7.04 (m, 2H), 7.01 (d, J=7.6 Hz, 1H), 6.70 (d, J=2.8 Hz, 1H), 6.27 (s, 1H), 5.36-5.26 (m, 2H), 3.82 (d, J=2.0 Hz, 3H), 2.98-2.93 (m, 1H), 2.62-2.54 (m, 2H), 2.35-2.29 (m, 1H), 2.08-2.05 (m, 1H), 1.64 (s, 3H), 1.61-1.55 (m, 1H), 1.48-1.41 (m, 2H), 1.36-1.31 (m, 1H), 1.19-1.17 (m, 6H), 1.10-1.06 (m, 3H), 0.95-0.90 (m, 2H) ppm.

### Example 103. Compound 103. 3-[3-(23,29-difluoro-3,6,11,11,12-pentamethyl-13-oxo-14,25-dioxa-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-propanoic acid

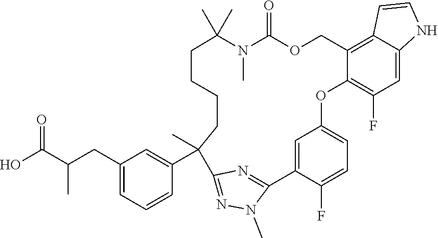 [SMILES: CC(Cc1cccc(C2(C)CCCCC(C)(C)N(C)C(=O)OCc3c(c(F)cc4[nH]ccc34)Oc3ccc(F)c(c3)-c3nc2nn3C)c1)C(=O)O]

### (5-(3-(3-(2-(3-Bromophenyl)pent-4-en-2-yl)-1-methyl-1H-1,2,4-triazol-5-yl)-4-fluorophenoxy)-6-fluoro-1 -(phenylsulfonyl)-1H-indol-4-yl)methyl acetate

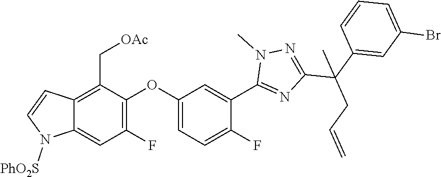

Step A: To a stirred solution of 2-(3-bromophenyl)-N′,2-dimethylpent-4-enehydrazide (Intermediate 67-8, 500 mg, 1.68 mmol) in pyridine (20 mL) was added (6-fluoro-5-(4-fluoro-3-(imino(methylthio)methyl)phenoxy)-1-(phenylsulfonyl)-1H-indol-4-yl)methyl acetate hydroiodide (Intermediate 65-2, 1.03 g, 1.68 mmol) and magnesium sulfate (2 g). The mixture was stirred at 80° C. overnight, cooled to room temperature, and quenched with 50 mL of water. The solution was neutralized to pH˜7 with hydrochloric acid (1 N) and extracted with ethyl acetate (3×50 mL). The combined organic phases were washed with brine, dried over sodium sulfate and concentrated. The residue was purified by silica gel column chromatography (eluting with petroleum ether: ethyl acetate=2:1) to give the title compound (840 mg, 59%) as a yellow solid. MS (ESI): 761, 763 m/z [M+H]⁺, retention time: 2.43 minutes, purity: 74% (214 nm) (LC-MS method 4).

### (5-(3-(3-(2-(3-Bromophenyl)pent-4-en-2-yl)-1-methyl-1H-1,2,4-triazol-5-yl)-4-fluorophenoxy)-6-fluoro-1 -(phenylsulfonyl)-1H-indol-4-yl)methanol

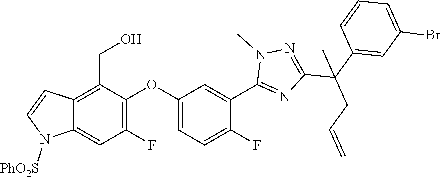

Step B: To a stirred solution of Step A product (840 mg, 0.999 mmol) in methanol (20 mL) was added potassium carbonate (276 mg, 2.00 mmol). The mixture was stirred at room temperature for 2 hours, then poured into water (200 mL). The formed solid was collected by filtration and the filter cake was dried to afford the crude title compound (760 mg, crude) as a white solid. MS (ESI): 719, 721 m/z [M+H]⁺, retention time: 2.13 minutes, purity: 74% (214 nm) (LC-MS method 25).

### (5-(3-(3-(2-(3-Bromophenyl)pent-4-en-2-yl)-1-methyl-1H-1,2,4-triazol-5-yl)-4-fluorophenoxy)-6-fluoro-1 -(phenylsulfonyl)-1H-indol-4-yl)methyl (4-nitrophenyl) carbonate

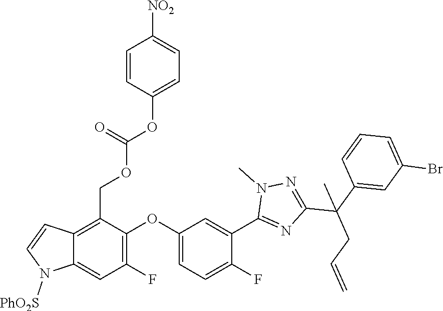

Step C: To a stirred solution of Step B product (760 mg, 0.952 mmol) in dichloromethane (30 mL) was added 4-nitrophenyl carbonochloridate (384 mg, 1.90 mmol) and triethylamine (289 mg, 2.86 mmol). The mixture was stirred at room temperature overnight, quenched with 80 mL of water, extracted with ethyl acetate (3×100 mL). The combined organic phases were washed with brine, dried over sodium sulfate and concentrated. The residue was purified by silica gel column chromatography (eluting with petroleum ether: ethyl acetate=2:1) to give the title compound (700 mg, 76%) as a white solid. MS (ESI): 884, 886 m/z [M+H]⁺, retention time: 2.25 minutes, purity: 92% (214 nm) (LC-MS method 25).

### (5-(3-(3-(2-(3-Bromophenyl)pent-4-en-2-yl)-1-methyl-1H-1,2,4-triazol-5-yl)-4-fluorophenoxy)-6-fluoro-1 -(phenylsulfonyl)-NH-indol-4-yl)methyl methyl(2-methylpent-4-en-2-yl)carbamate

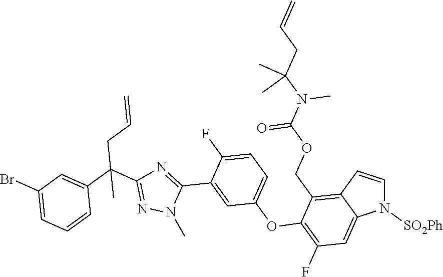

Step D: To a stirred solution of Step C product (700 mg, 0.726 mmol) in N,N-dimethylformamide (10 mL) was added N,2-dimethylpent-4-en-2-amine hydrochloride (Intermediate 68, 247 mg, 2.18 mmol) and triethylamine (368 mg, 3.63 mmol). The mixture was stirred at 60° C. for 3 hours, cooled to room temperature, and quenched with 30 mL of water. The solution was extracted with ethyl acetate (3×30 mL). The combined organic phases were washed with brine, dried over sodium sulfate and concentrated. The residue was purified by silica gel column chromatography (eluting with petroleum ether: ethyl acetate=2:1) to give the title compound (380 mg, 56%) as a yellow solid. MS (ESI): 858, 860 m/z [M+H]⁺, retention time: 2.47 minutes, purity: 78% (214 nm) (LC-MS method 25).

### (8Z)-20-(benzenesulfonyl)-6-(3-bromophenyl)-23,29-difluoro-3,6,11,11,12-pentamethyl-14,25-dioxa-3,4,12,20,31-pentazapentacyclo[24.3.1.12,5.016,24.017,21]hentriaconta-1 (30),2(31),4,8,16,18,21,23,26,28-decaen-13-one

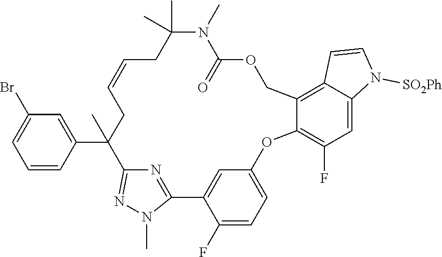

Step E: To a stirred solution of Step D product (380 mg, 0.405 mmol) in toluene (80 mL) was added Grubbs catalyst second generation (138 mg, 0.162 mmol). The mixture was stirred at 60° C. for 2 days and concentrated. The residue was diluted with 50 mL of water and extracted with ethyl acetate (3×50 mL). The combined organic phases were washed with brine, dried over sodium sulfate and concentrated. The crude product was purified by Prep-TLC (developing with petroleum ether: ethyl acetate=2:1) to give the title compound (220 mg, 60%) as a white solid. MS (ESI): 830, 832 m/z [M+H]⁺, retention time: 2.32 minutes, purity: 40% (214 nm) (LC-MS method 25).

### (8Z)-6-(3-Bromophenyl)-23,29-difluoro-3,6,11,11,12-pentamethyl-14,25-dioxa-3,4,12,20,31-pentazapentacyclo[24.3.1.12,5.016,24.017,21]hentriaconta-1(30),2(31),4,8,16,18,21,23,26,28-decaen-13-one

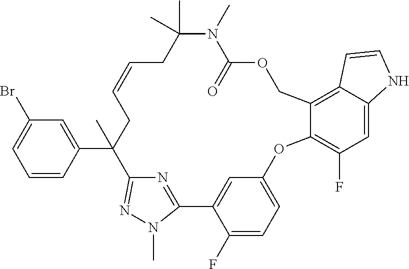 [SMILES: CN1C(=O)OCc2c(c(F)cc3[nH]ccc23)Oc2ccc(F)c(c2)-c2nc(nn2C)C(C)(c2cccc(Br)c2)C/C=C\CC1(C)C]

Step F: To a stirred solution of Step E product (220 mg, 0.242 mmol) in tetrahydrofuran (15 mL) was added lithium hydroxide monohydrate (51 mg, 1.21 mmol). The mixture was stirred at room temperature overnight, quenched with 20 mL of water, and extracted with ethyl acetate (3×20 mL). The combined organic phases were washed with brine, dried over sodium sulfate and concentrated. The residue was purified by Prep-TLC (developing with dichloromethane:methanol=30:1) to give the title compound (95 mg, 57%) as a white solid. MS (ESI): 690, 692 m/z [M+H]⁺, retention time: 2.16 minutes, purity: 58% (214 nm) (LC-MS method 25).

### Ethyl (E)-3-[3-[(8Z)-23,29-difluoro-3,6,11,11,12-pentamethyl-13-oxo-14,25-dioxa-3,4,12,20,31-pentazapentacyclo[24.3.1.12,5.016,24.017,21]hentriaconta-1(30),2(31),4,8,16,18,21,23,26,28-decaen-6-yl]phenyl]-2-methyl-prop-2-enoate

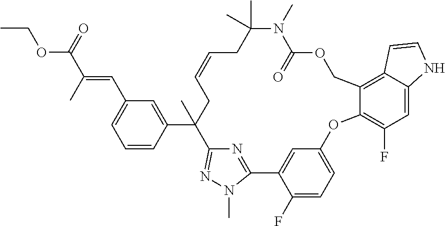 [SMILES: CCOC(=O)/C(C)=C/c1cccc(C2(C)C/C=C\CC(C)(C)N(C)C(=O)OCc3c(c(F)cc4[nH]ccc34)Oc3ccc(F)c(c3)-c3nc2nn3C)c1]

Step G: To a stirred solution of Step F product (95 mg, 0.138 mmol) in 1,4-dioxane (8 mL) and water (1.5 mL) was added ethyl (E)-2-methyl-3-(4,4,5,5-tetramethyl-1,3,2-dioxaborolan-2-yl)prop-2-enoate (66.1 mg, 0.275 mmol), 1,1′-bis(diphenylphosphino)ferrocene-palladium(ii)dichloride dichloromethane complex (11.2 mg, 0.0138 mmol) and cesium carbonate (134 mg, 0.413 mmol). The mixture was stirred at 100° C. overnight, cooled to room temperature, and quenched with 20 mL of water. The solution was extracted with ethyl acetate (3×20 mL). The combined organic extracts were washed with brine, dried over sodium sulfate and concentrated. The residue was purified by Prep-TLC (developing with dichloromethane:methanol=30:1) to give the title compound (85 mg, 85%) as a white solid. MS (ESI): 724 m/z [M+H]⁺, retention time: 2.21 minutes, purity: 60% (214 nm) (LC-MS method 25).

### Ethyl 3-[3-(23,29-difluoro-3,6,11,11,12-pentamethyl-13-oxo-14,25-dioxa-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-propanoate

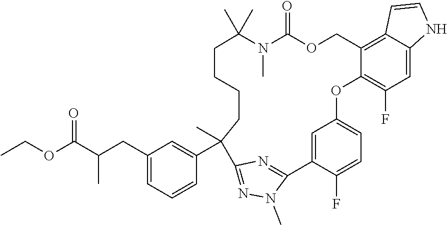 [SMILES: CCOC(=O)C(C)Cc1cccc(C2(C)CCCCC(C)(C)N(C)C(=O)OCc3c(c(F)cc4[nH]ccc34)Oc3ccc(F)c(c3)-c3nc2nn3C)c1]

Step H: To a stirred solution of Step G product (85 mg, 0.117 mmol) in methanol (8 mL) was added palladium on carbon (50% wet, 10%, 12.5 mg, 0.0117 mmol). The mixture was stirred at room temperature under hydrogen balloon for 2 hours. The mixture was filtered through a pad of Celite. The filtrate was concentrated to give the crude title compound (75 mg, 88%) as a white solid. MS (ESI): 728 m/z [M+H]⁺, retention time: 2.21 minutes, purity: 66% (214 nm) (LC-MS method 25).

### Compound 103. 3-[3-(23,29-difluoro-3,6,11,11,12-pentamethyl-13-oxo-14,25-dioxa-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-propanoic acid

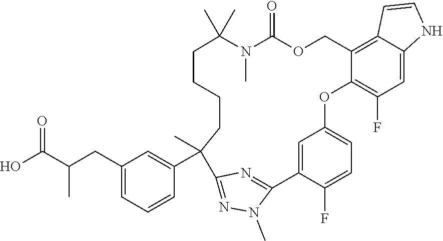 [SMILES: CC(Cc1cccc(C2(C)CCCCC(C)(C)N(C)C(=O)OCc3c(c(F)cc4[nH]ccc34)Oc3ccc(F)c(c3)-c3nc2nn3C)c1)C(=O)O]

Step I: To a stirred solution of Step H product (75 mg, 0.103 mmol) in methanol (8 mL) and water (2 mL) was added lithium hydroxide monohydrate (21.6 mg, 0.515 mmol). The mixture was stirred at room temperature for 3 hours, quenched with 15 mL of water, and acidified to pH˜4 with hydrochloric acid solution (1 N). The mixture was extracted with ethyl acetate (3×20 mL). The combined organic phases were washed with brine, dried over sodium sulfate and concentrated. The residue was purified by Prep-TLC (developing with dichloromethane:methanol=30:1) to give the title compound (44.2 mg, 61%) as a white solid. MS (ESI): 700 m/z [M+H]⁺, retention time: 2.01 minutes, purity: 97% (214 nm) (LC-MS method 25). ¹H NMR (400 MHz, CD₃OD) δ 7.34-7.27 (m, 4H), 7.17-7.09 (m, 3H), 7.06-7.00 (m, 2H), 6.67 (d, J=2.8 Hz, 1H), 5.35 (s, 2H), 3.84 (d, J=2.4 Hz, 3H), 3.01-2.96 (m, 1H), 2.73 (s, 3H), 2.60-2.52 (m, 2H), 2.32-2.27 (m, 1H), 2.11-2.02 (m, 1H), 1.74-1.70 (m, 1H), 1.63 (s, 3H), 1.54-1.44 (m, 2H), 1.30 (s, 3H), 1.28 (s, 3H), 1.19-1.06 (m, 1H), 1.07-1.04 (m, 3H), 0.93-0.88 (m, 2H) ppm.

### Example 104. Compound 104. 3-[3-(23,29-Difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

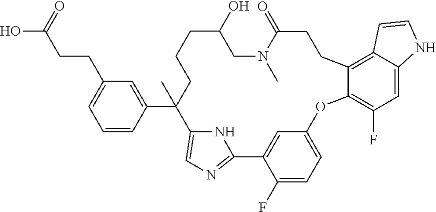 [SMILES: CN1CC(O)CCCC(C)(c2cccc(CCC(=O)O)c2)c2cnc([nH]2)-c2cc(ccc2F)Oc2c(F)cc3[nH]ccc3c2CCC1=O]

### Ethyl 3-[3-(23,29-difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate

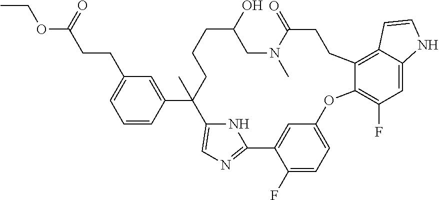 [SMILES: CCOC(=O)CCc1cccc(C2(C)CCCC(O)CN(C)C(=O)CCc3c(c(F)cc4[nH]ccc34)Oc3ccc(F)c(c3)-c3ncc2[nH]3)c1]

Step A: Exchanging 5-((4-(3-azidopropyl)-6-fluoro-1H-indol-5-yl)oxy)-2-fluorobenzimidamide (Intermediate 5-4) with ethyl (E)-3-(5-(3-carbamimidoyl-4-fluorophenoxy)-6-fluoro-1H-indol-4-yl)acrylate (Intermediate 5-2, 472 mg, 1.27 mmol), and 7-bromo-5-(3-iodophenyl)-6-oxoheptanenitrile (Intermediate 1) with 5-(6-chloro-4-(3-iodophenyl)-4-methyl-5-oxohexyl)-3-methyloxazolidin-2-one (Intermediate 1-44, 520 mg, 1.15 mmol), the reaction procedure sequence (Steps A, B of Example 11, followed by Steps E, C and Step F of Example 1, in this order) was used to prepare the title compound (54 mg, yellow oil). MS (ESI): 685 m/z [M+H]⁺, retention time: 1.78 minutes, purity: 88% (214 nm) (LC-MS method 3).

### Compound 104: 3-[3-(23,29-Difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

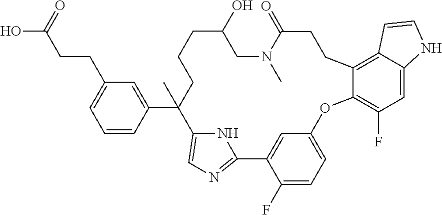 [SMILES: CN1CC(O)CCCC(C)(c2cccc(CCC(=O)O)c2)c2cnc([nH]2)-c2cc(ccc2F)Oc2c(F)cc3[nH]ccc3c2CCC1=O]

Step B: To a stirred solution of Step A product (35 mg, 88% purity, 0.045 mmol) in tetrahydrofuran-methanol-water (3:1:1) (1.5 mL) was added lithium hydroxide monohydrate (53 mg, 1.26 mmol). The mixture was stirred at room temperature for 3 hours and concentrated. The residue was dissolved in ice-water (2 mL), acidified with hydrochloric acid (3 M) to pH˜6, and extracted with ethyl acetate (2×15 mL). The combined organic extracts were washed with brine (20 mL). dried over sodium sulfate, and concentrated. The crude product was purified by prep-TLC (developing with 10% methanol in dichloromethane) to give the title compound (6.6 mg, 22%) as a white solid. MS (ESI): 657 m/z [M+H]⁺, retention time: 1.67 minutes, purity: 97% (214 nm) (LC-MS method 3). (mixture of four diastereomers, originated from two chiral centers).

### Example 105. Compound 105. 3-[3-(23,29-Difluoro-6,12-dimethyl-10,13-dioxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

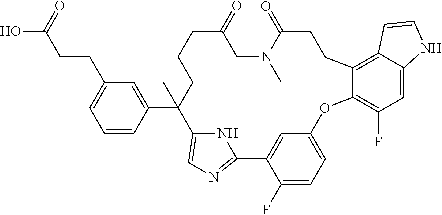 [SMILES: CN1CC(=O)CCCC(C)(c2cccc(CCC(=O)O)c2)c2cnc([nH]2)-c2cc(ccc2F)Oc2c(F)cc3[nH]ccc3c2CCC1=O]

### Ethyl 3-[3-(23,29-difluoro-6,12-dimethyl-10,13-dioxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate

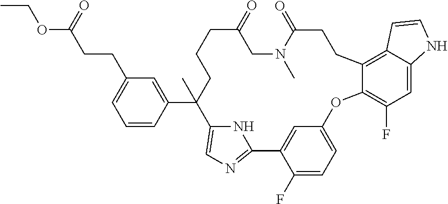 [SMILES: CCOC(=O)CCc1cccc(C2(C)CCCC(=O)CN(C)C(=O)CCc3c(c(F)cc4[nH]ccc34)Oc3ccc(F)c(c3)-c3ncc2[nH]3)c1]

Step A: To a stirred solution of ethyl 3-[3-(23,29-difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate (Step A product of Example 104, 37 mg, 88% purity, 0.0476 mmol) in dimethyl sulfoxide (2.5 mL) was added 2-Iodoxybenzoic acid (40 mg, 0.143 mmol). The mixture was stirred at room temperature for 18 hours and diluted with ethyl acetate (30 mL). The solution was washed with brine, dried over sodium sulfate, and concentrated. The residue was purified by prep-TLC (developing with 10% methanol in dichloromethane) to give the title compound (25 mg, 61%) as a light yellow oil. MS (ESI): 683 m/z [M+H]⁺, retention time: 1.78 minutes, purity: 90% (214 nm) (LC-MS method 3).

### Compound 105: 3-[3-(23,29-Difluoro-6,12-dimethyl-10,13-dioxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

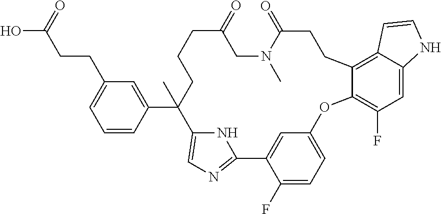 [SMILES: CN1CC(=O)CCCC(C)(c2cccc(CCC(=O)O)c2)c2cnc([nH]2)-c2cc(ccc2F)Oc2c(F)cc3[nH]ccc3c2CCC1=O]

Step B: Utilizing the identical conditions described in Step B of Example 104, the Step A product (35 mg, 90% purity, 0.033 mmol) was converted to the title compound (9.5 mg, 45%) as a light-yellow solid. MS (ESI): 655 m/z [M+H]⁺, retention time: 1.69 minutes, purity: 99% (214 nm) (LC-MS method 3). ¹H NMR (400 MHz, CD₃OD) δ 7.96-7.93 (m, 1H), 7.30-7.07 (m, 8H), 6.95-6.63 (m, 1H), 6.52 (dd, J=12.0, 3.2 Hz, 1H), 4.24-4.20 (m, 1H), 4.09 (d, J=5.2 Hz, 1H), 3.20-3.00 (m, 2H), 2.97-2.93 (m, 2H), 2.92-2.77 (m, 2H), 2.77-2.74 (m, 2H), 2.58-2.43 (m, 3H), 2.28-1.85 (m, 3H), 1.65-1.61 (m, 2H), 1.53-1.48 (m, 1H), 1.42-1.27 (m, 3H) ppm.

### Example 106. Compound 106. 3-[3-(23,29-Difluoro-10-hydroxy-6-methyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

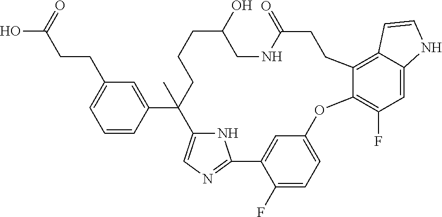 [SMILES: CC1(c2cccc(CCC(=O)O)c2)CCCC(O)CNC(=O)CCc2c(c(F)cc3[nH]ccc23)Oc2ccc(F)c(c2)-c2ncc1[nH]2]

### Ethyl 3-[3-(23,29-difluoro-10-hydroxy-6-methyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate

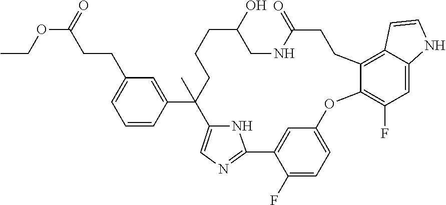 [SMILES: CCOC(=O)CCc1cccc(C2(C)CCCC(O)CNC(=O)CCc3c(c(F)cc4[nH]ccc34)Oc3ccc(F)c(c3)-c3ncc2[nH]3)c1]

Step A: Exchanging 8-bromo-6-(3-iodophenyl)-7-oxooctanenitrile (Intermediate 1-1) with 1-azido-8-bromo-6-(3-iodophenyl)-6-methyl-7-oxooctan-2-yl acetate (Intermediate 1-45, 1 g, 1.91 mmol), the reaction procedure sequence (Steps A, B of Example 1, followed by Step C of Example 11, then Steps E, C and Step F of Example 1, in this order) was used to prepare the title compound (16 mg, yellow oil) as a white solid. MS (ESI): 685 m/z [M+H]⁺, retention time: 1.96 minutes, purity: 95% (214 nm) (LC-MS method 3).

### Compound 106. 3-[3-(23,29-Difluoro-10-hydroxy-6-methyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

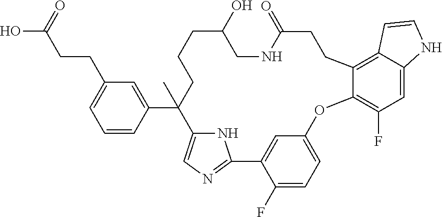 [SMILES: CC1(c2cccc(CCC(=O)O)c2)CCCC(O)CNC(=O)CCc2c(c(F)cc3[nH]ccc23)Oc2ccc(F)c(c2)-c2ncc1[nH]2]

Step B: Utilizing the identical conditions described in Step B of Example 104, the Step A product (20 mg, 90% purity, 0.033 mmol) was converted to the title compound (16 mg, 80%) as a white solid. MS (ESI): 643 m/z [M+H]⁺, retention time: 1.86 minutes, purity: 95% (214 nm) (LC-MS method 3). ¹H NMR (400 MHz, CD₃OD) δ 7.84-7.83 (m, 1H), 7.31-7.27 (m, 2H), 7.26-7.24 (m, 1H), 7.23-7.19 (m, 3H), 7.16-7.14 (m, 1H), 7.12-7.11 (m, 1H), 7.08-7.06 (m, 1H), 6.58-6.56 (d, 1H), 3.36-3.32 (m, 1H), 3.08-3.06 (m, 2H), 2.91-2.86 (m, 2H), 2.61-2.54 (m, 2H), 2.49-2.47 (m, 2H), 2.47-2.45 (m, 2H), 1.64 (s, 3H), 1.60-1.50 (m, 2H), 1.40-1.35 (m, 4H) ppm.

### Example 107. Compound 107. 3-[3-(23,29-Difluoro-6-methyl-10,13-dioxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoic acid

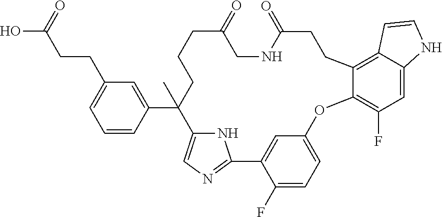 [SMILES: CC1(c2cccc(CCC(=O)O)c2)CCCC(=O)CNC(=O)CCc2c(c(F)cc3[nH]ccc23)Oc2ccc(F)c(c2)-c2ncc1[nH]2]

Exchanging ethyl 3-[3-(23,29-difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate (Step A product of Example 104) with ethyl 3-[3-(23,29-difluoro-10-hydroxy-6-methyl-13-oxo-25-oxa-3,12,20,31-tetrazapentacyclo-[24.3.1.12,5.016,24.017,21]hentriaconta-1(30),2,4,16,18,21,23,26,28-nonaen-6-yl)phenyl]propanoate (Step A product of Example 106, 60 mg, 0.09 mmol), the reaction procedure sequence (Steps A and B) described for Example 105 was used to prepare the title compound (16 mg) as a white solid. MS (ESI): 641 m/z [M+H]⁺, retention time: 1.86 minutes, purity: 95% (214 nm) (LC-MS method 3). ¹H NMR (400 MHz, CD₃OD) δ 8.21-8.19 (m, 1H), 7.67 (s, 1H), 7.34-7.31 (m, 3H), 7.29-7.27 (m, 2H), 7.23-7.20 (m, 2H), 7.04-7.01 (m, 1H), 6.54-6.51 (m, 1H), 4.07-4.01 (m, 2H), 3.02-3.00 (m, 2H), 2.93-2.90 (m, 2H), 2.63-2.55 (m, 7H), 2.07-2.05 (m, 1H), 1.67 (s, 3H), 1.47-1.45 (m, 2H) ppm.

### Example 108. Compound 108. 3-[3-(24,30-Difluoro-12-hydroxy-6-methyl-15-oxo-26-oxa-3,14,21,32-tetrazapentacyclo[25.3.1.12,5.017,25.018,22]dotriaconta-1(31),2,4,17,19,22,24,27,29-nonaen-6-yl)phenyl]propanoic acid

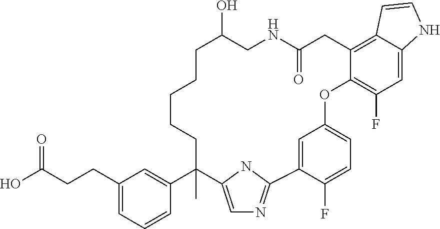 [SMILES: CC1(c2cccc(CCC(=O)O)c2)CCCCCC(O)CNC(=O)Cc2c(c(F)cc3[nH]ccc23)Oc2ccc(F)c(c2)-c2ncc1[nH]2]

### Ethyl 3-[3-(24,30-difluoro-12-hydroxy-6-methyl-15-oxo-26-oxa-3,14,21,32-tetrazapentacyclo[25.3.1.12,5.017,25.018,22]dotriaconta-1(31),2,4,17,19,22,24,27,29-nonaen-6-yl)phenyl]propanoate

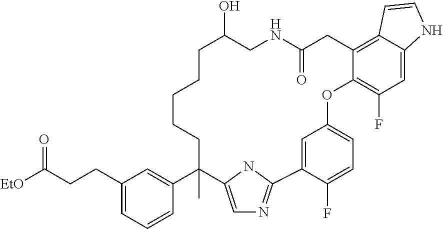 [SMILES: CCOC(=O)CCc1cccc(C2(C)CCCCCC(O)CNC(=O)Cc3c(c(F)cc4[nH]ccc34)Oc3ccc(F)c(c3)-c3ncc2[nH]3)c1]

Step A: Exchanging methyl (E)-3-(5-(3-carbamimidoyl-4-fluorophenoxy)-6-fluoro-1H-indol-4-yl)acrylate (Intermediate 5) with ethyl 2-(5-(3-carbamimidoyl-4-fluorophenoxy)-6-fluoro-1H-indol-4-yl)acetate (Intermediate 6, 1.6 g, 4.35 mmol), and 8-bromo-6-(3-iodophenyl)-7-oxooctanenitrile (Intermediate 1-1) with 1-azido-10-bromo-8-(3-iodophenyl)-8-methyl-9-oxodecan-2-yl acetate (Intermediate 1-46, 2.4 g, 4.35 mmol), the reaction procedure sequence (Steps A, B of Example 1, followed by Step C of Example 11, then Steps E, C, and Step F of Example 1, in this order) was used to prepare the title compound (170 mg, brown oil). MS (ESI): 685 m/z [M+H]⁺, retention time: 1.54 minutes, purity: 83% (214 nm) (LC-MS method 3). (Mixture of 4 diastereomers, originating from two chiral centers)

### Compound 108. 3-[3-(24,30-Difluoro-12-hydroxy-6-methyl-15-oxo-26-oxa-3,14,21,32-tetrazapentacyclo[25.3.1.12,5.017,25.018,22]dotriaconta-1(31),2,4,17,19,22,24,27,29-nonaen-6-yl)phenyl]propanoic acid

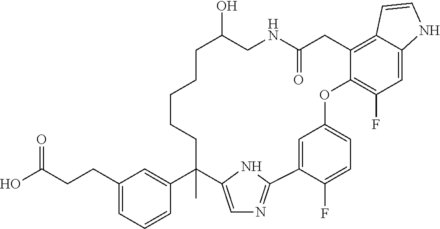 [SMILES: CC1(c2cccc(CCC(=O)O)c2)CCCCCC(O)CNC(=O)Cc2c(c(F)cc3[nH]ccc23)Oc2ccc(F)c(c2)-c2ncc1[nH]2]

Step B: To a stirred and cooled (0° C.) solution of Step A product (50 mg, 0.073 mmol) in tetrahydrofuran (3 mL) and methanol (1 mL) was added lithium hydroxide monohydrate (8.8 mg, 0.365 mmol). The solution was stirred at room temperature for 3 hours and concentrated. The residue was dissolved in water (3 mL), acidified to pH˜6 with 1 N hydrochloric acid. The formed solid was collected by filtration and dried to afford the title compound (18.3 mg, 50%) as a white solid. MS (ESI): 657 m/z [M+H]⁺, retention time: 1.71 minutes, purity: 97% (214 nm) (LC-MS method 3) (mixture of two diastereomers, originated from two chiral center). ¹H NMR (400 MHz, CD₃OD) δ 7.38 (d, J=2.8 Hz, 1H), 7.26-6.76 (m, 9H), 6.44-6.40 (m, 1H), 3.76-3.66 (m, 2H), 2.82-2.68 (m, 4H), 2.43-2.39 (m, 2H), 2.16-2.12 (m, 1H), 1.96-1.90 (m, 1H), 1.46-1.41 (m, 3H), 1.32-1.03 (m, 9H) ppm.

### Example 109. Compound 109.3-[3-(24,30-Difluoro-6-methyl-12,15-dioxo-26-oxa-3,14,21,32-tetrazapentacyclo[25.3.1.12,5.017,25.018,22]dotriaconta-1(31),2,4,17,19,22,24,27,29-nonaen-6-yl)phenyl]propanoic acid

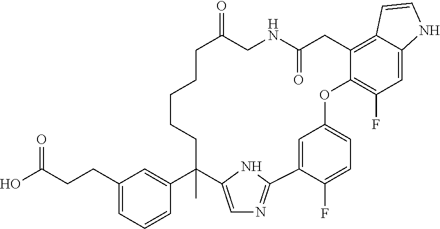 [SMILES: CC1(c2cccc(CCC(=O)O)c2)CCCCCC(=O)CNC(=O)Cc2c(c(F)cc3[nH]ccc23)Oc2ccc(F)c(c2)-c2ncc1[nH]2]

Exchanging ethyl 3-[3-(23,29-difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate (Step A product of Example 104) with 3-[3-(24,30-difluoro-12-hydroxy-6-methyl-15-oxo-26-oxa-3,14,21,32-tetrazapentacyclo[25.3.1.12,5.017,25.018,22]-dotriaconta-1(31),2,4,17,19,22,24,27,29-nonaen-6-yl)phenyl]propanoic acid (Step A product of Example 108, 170 mg, 0.26 mmol), the reaction procedure sequence (Steps A and B) described for Example 108 was used to prepare the title compound (5 mg, 16% two steps; only 20 mg out of 100 mg obtained from corresponding Step A was used in Step B) as a white solid. MS (ESI): 655 m/z [M+H]⁺, retention time: 1.70 minutes, purity: 97% (214 nm) (LC-MS method 3). ¹H NMR (400 MHz, CD₃OD) δ 7.28-6.85 (m, 10H), 6.46 (d, J=3.2 Hz, 1H), 3.80-3.75 (m, 2H), 3.64-3.59 (m, 1H), 3.43-3.37 (m, 1H), 2.71 (t, J=7.7 Hz, 2H), 2.38 (t, J=7.7 Hz, 2H), 2.18-2.12 (m, 1H), 2.02-1.92 (m, 3H), 1.59-1.14 (m, 9H) ppm.

### Example 110. Compound 110. 3-[3-(24,30-Difluoro-15-hydroxy-6-methyl-12-oxo-26-oxa-3,13,21,32-tetrazapentacyclo[25.3.1.12,5.017,25.018,22]dotriaconta-1(31),2,4,17,19,22,24,27,29-nonaen-6-yl)phenyl]propanoic acid

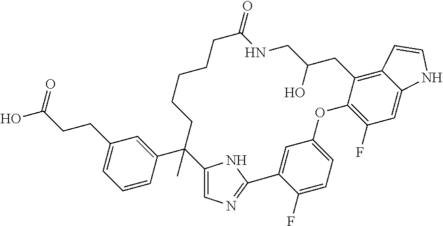 [SMILES: CC1(c2cccc(CCC(=O)O)c2)CCCCCC(=O)NCC(O)Cc2c(c(F)cc3[nH]ccc23)Oc2ccc(F)c(c2)-c2ncc1[nH]2]

### Ethyl 3-[3-(24,30-Difluoro-15-hydroxy-6-methyl-12-oxo-26-oxa-3,13,21,32-tetrazapentacyclo[25.3.1.12,5.017,25.018,22]dotriaconta-1(31),2,4,17,19,22,24,27,29-nonaen-6-yl)phenyl]propanoate

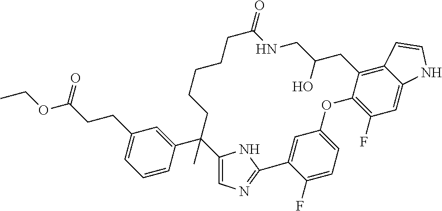 [SMILES: CCOC(=O)CCc1cccc(C2(C)CCCCCC(=O)NCC(O)Cc3c(c(F)cc4[nH]ccc34)Oc3ccc(F)c(c3)-c3ncc2[nH]3)c1]

Step A: Exchanging 7-bromo-5-(3-iodophenyl)-6-oxoheptanenitrile (Intermediate 1) with 9-bromo-7-(3-iodophenyl)-7-methyl-8-oxononanenitrile (Intermediate 1-46, 1.459 g, 3.780 mmol), and 5-((4-(3-azidopropyl)-6-fluoro-1H-indol-5-yl)oxy)-2-fluorobenzimidamide (Intermediate 5-4) with 2-fluoro-5-((6-fluoro-4-((2-oxooxazolidin-5-yl)methyl)-1H-indol-5-yl)oxy)benzimidamide (Intermediate 61, 1.408 g, 3.150 mmol), the reaction procedure sequence (Steps A, B of Example 11, followed by Steps E, C, F of Example 1) was used to prepare the title compound (145 mg). MS (ESI): 685 m/z [M+H]⁺, retention time: 1.76 minutes, purity: 86% (214 nm) (LC-MS method 2). (mixture of four diastereomers, originated from two chiral centers).

### Compound 110. 3-[3-(24,30-Difluoro-15-hydroxy-6-methyl-12-oxo-26-oxa-3,13,21,32-tetrazapentacyclo[25.3.1.12,5.017,25.018,22]dotriaconta-1(31),2,4,17,19,22,24,27,29-nonaen-6-yl)phenyl]propanoic acid

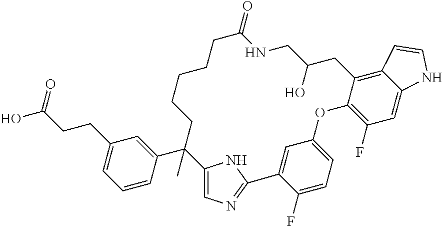 [SMILES: CC1(c2cccc(CCC(=O)O)c2)CCCCCC(=O)NCC(O)Cc2c(c(F)cc3[nH]ccc23)Oc2ccc(F)c(c2)-c2ncc1[nH]2]

Step B: To a solution of Step A product (0.025 g, 0.038 mmol) in tetrahydrofuran/methanol/water (2:2:1 (v/v/v), 5 ml) was added lithium hydroxide (7.7 mg, 0.19 mmol). The mixture was stirred at room temperature for 2 hours, diluted with water (20 mL), and acidified to pH˜5-6 with 1N hydrochloric acid. The mixture was extracted with ethyl acetate (3×20 mL). The combined organic phases were washed with water, brine, dried over sodium sulfate, and concentrated. The residue was purified by prep-TLC (dichloromethane/methanol=20/1) to give the title compound (10 mg, 42%) as a white solid. MS (ESI): 657 m/z [M+H]⁺, retention time: 1.30 minutes, purity: >99% (254 nm) (LC-MS method 27). (mixture of four diastereomers, originated from two chiral centers).

### Example 111. Compound 111. 3-[3-(24,30-Difluoro-6-methyl-12,15-dioxo-26-oxa-3,13,21,32-tetrazapentacyclo[25.3.1.12,5.017,25.018,22]dotriaconta-1(31),2,4,17,19,22,24,27,29-nonaen-6-yl)phenyl]propanoic acid

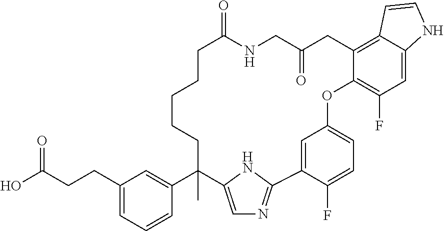 [SMILES: CC1(c2cccc(CCC(=O)O)c2)CCCCCC(=O)NCC(=O)Cc2c(c(F)cc3[nH]ccc23)Oc2ccc(F)c(c2)-c2ncc1[nH]2]

Exchanging ethyl 3-[3-(23,29-difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate (Step A product of Example 104) with ethyl 3-[3-(24,30-Difluoro-15-hydroxy-6-methyl-12-oxo-26-oxa-3,13,21,32-tetrazapentacyclo[25.3.1.12,5.017,25.018,22]dotriaconta-1(31),2,4,17,19,22,24,27,29-nonaen-6-yl)phenyl]propanoate (Step A product of 110, 50 mg, 0.07 mmol), the reaction procedure sequence (Steps A and V) described for Example 105 was used to prepare the title compound (5 mg, 10% two steps) as a white solid. MS (ESI): 655 m/z [M+H]⁺, retention time: 1.31 minutes, purity: >99% (214 nm) (LC-MS method 27). ¹H NMR (400 MHz, CD₃OD) δ 7.32-7.28 (m, 2H), 7.23-7.15 (m, 2H), 7.14-7.08 (m, 3H), 7.04 (s, 1H), 7.02-6.93 (m, 2H), 6.46 (d, J=3.1 Hz, 1H), 4.21-4.15 (m, 2H), 4.07-4.01 (m, 2H), 2.82 (t, J=7.8 Hz, 2H), 2.46 (t, J=7.8 Hz, 2H), 2.25-2.20 (m, 2H), 2.13-2.08 (m, 2H), 1.66-1.60 (m, 1H), 1.53 (s, 3H), 1.50-1.29 (m, 4H), 0.93-0.89 (m, 1H) ppm.

### Example 112. Compound 112. 3-[3-(24,30-Difluoro-12-hydroxy-6,14-dimethyl-15-oxo-26-oxa-3,14,21,32-tetrazapentacyclo[25.3.1.12,5.017,25.018,22]dotriaconta-1(30),2,4,17(25),18(22),19,23,27(31),28-nonaen-6-yl)phenyl]propanoic acid

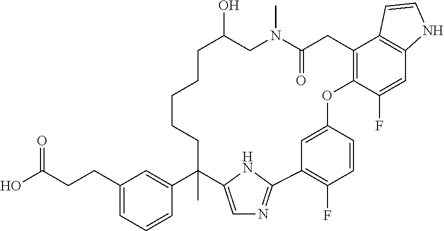 [SMILES: CN1CC(O)CCCCCC(C)(c2cccc(CCC(=O)O)c2)c2cnc([nH]2)-c2cc(ccc2F)Oc2c(F)cc3[nH]ccc3c2CC1=O]

### Ethyl 3-[3-(24,30-difluoro-12-hydroxy-6,14-dimethyl-15-oxo-26-oxa-3,14,21,32-tetrazapentacyclo[25.3.1.12,5.017,25.018,22]dotriaconta-1(30),2,4,17(25),18(22),19,23,27(31),28-nonaen-6-yl)phenyl]propanoate

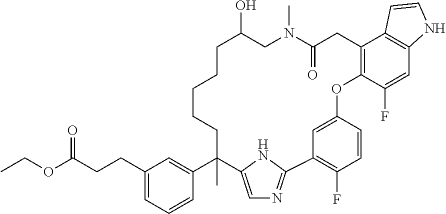 [SMILES: CCOC(=O)CCc1cccc(C2(C)CCCCCC(O)CN(C)C(=O)Cc3c(c(F)cc4[nH]ccc34)Oc3ccc(F)c(c3)-c3ncc2[nH]3)c1]

Step A: Exchanging 5-((4-(3-azidopropyl)-6-fluoro-1H-indol-5-yl)oxy)-2-fluorobenzimidamide (Intermediate 5-4) with ethyl 2-(5-(3-carbamimidoyl-4-fluorophenoxy)-6-fluoro-1H-indol-4-yl)acetate (Intermediate 6, 500 mg, 1.34 mmol), and 7-bromo-5-(3-iodophenyl)-6-oxoheptanenitrile (Intermediate 1) with 5-(8-chloro-6-(3-iodophenyl)-6-methyl-7-oxooctyl)-3-methyloxazolidin-2-one (Intermediate 1-48, 639 mg, 1.34 mmol), the reaction procedure sequence (Steps A and B of Example 11, followed by Steps E, C, and F of Example 1, in this order) was used to prepare the title compound (112 mg) as a white solid. MS (ESI): 699 m/z [M+H]⁺, retention time: 1.79 minutes, purity: 82% (254 nm) (LC-MS method 7). (mixture of two diastereomers, originated from two chiral centers).

### Compound 112. 3-[3-(24,30-difluoro-12-hydroxy-6,14-dimethyl-15-oxo-26-oxa-3,14,21,32-tetrazapentacyclo[25.3.1.12,5.017,25.018,22]dotriaconta-1(30),2,4,17(25),18(22),19,23,27(31),28-nonaen-6-yl)phenyl]propanoic acid

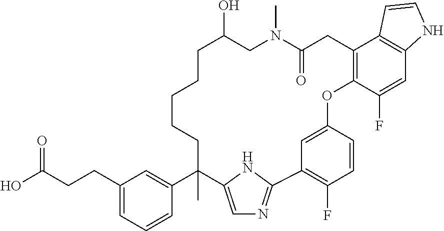 [SMILES: CN1CC(O)CCCCCC(C)(c2cccc(CCC(=O)O)c2)c2cnc([nH]2)-c2cc(ccc2F)Oc2c(F)cc3[nH]ccc3c2CC1=O]

Step B: Utilizing the identical reaction conditions described in Step B of Example 104, the title compound (40 mg, 30%) as a white solid. MS (ESI): 671 m/z [M+H]⁺, retention time: 1.33 minutes, purity: 96% (254 nm) (LC-MS method 2). (Mixture of four diastereomers, originated from two chiral centers).

### Example 113. Compound 113. 3-[3-(24,30-Difluoro-6,14-dimethyl-12,15-dioxo-26-oxa-3,14,21,32-tetrazapentacyclo[25.3.1.12,5.017,25.018,22]dotriaconta-1(30),2,4,17(25),18(22),19,23,27(31),28-nonaen-6-yl)phenyl]propanoic acid

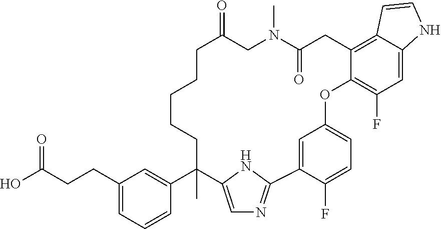 [SMILES: CN1CC(=O)CCCCCC(C)(c2cccc(CCC(=O)O)c2)c2cnc([nH]2)-c2cc(ccc2F)Oc2c(F)cc3[nH]ccc3c2CC1=O]

Exchanging ethyl 3-[3-(23,29-difluoro-10-hydroxy-6,12-dimethyl-13-oxo-25-oxa-3,12,20,31-tetrazapentacyclo[24.3.1.12,5.016,24.017,21]hentriaconta-1(30),2,4,16,18,21,23,26,28-nonaen-6-yl)phenyl]propanoate (Step A product of Example 104) with ethyl 3-[3-(24,30-difluoro-12-hydroxy-6,14-dimethyl-15-oxo-26-oxa-3,14,21,32-tetrazapentacyclo-[25.3.1.12,5.017,25.018,22]dotriaconta-1(30),2,4,17(25),18(22),19,23,27(31),28-nonaen-6-yl)phenyl]propanoate (Step A product of Example 112, 50 mg, 0.072 mg), the reaction procedure sequence (Steps A and B) described for Example 104 was used to prepare the title compound (6.2 mg) as a white solid. MS (ESI): 669 m/z [M+H]⁺, retention time: 1.36 minutes, purity: >99% (214 nm), >99% (254 nm) (LC-MS method 2).

### Example 114. Compound 114. 3-[3-(23,29-Difluoro-3,6-dimethyl-11,11,13-trioxo-25-oxa-11)₆-thia-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-propanoic acid

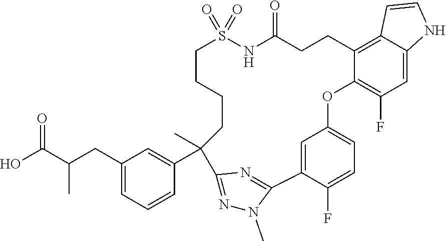 [SMILES: CC(Cc1cccc(C2(C)CCCCS(=O)(=O)NC(=O)CCc3c(c(F)cc4[nH]ccc34)Oc3ccc(F)c(c3)-c3nc2nn3C)c1)C(=O)O]

### Example 115. Compound 115.23,29-Difluoro-3,6-dimethyl-11,11-dioxo-6-phenyl-25-oxa-11)₆-thia-3,4,12,20,31-pentazapentacyclo[24.3.1.12,5.016,24.017,21]hentriaconta-1(30),2(31),4,16,18,21,23,26,28-nonaen-13-one

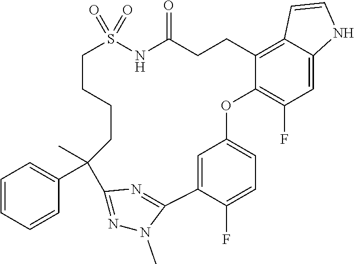 [SMILES: Cn1nc2nc1-c1cc(ccc1F)Oc1c(F)cc3[nH]ccc3c1CCC(=O)NS(=O)(=O)CCCCC2(C)c1ccccc1]

### Ethyl 3-(5-(3-(3-(2-(3-bromophenyl)pent-4-en-2-yl)-1-methyl-1H-1,2,4-triazol-5-yl)-4-fluorophenoxy)-6-fluoro-1H-indol-4-yl)propanoate

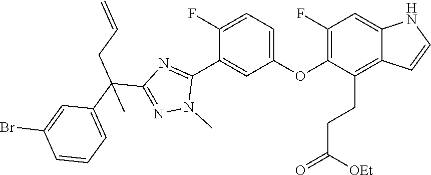 [SMILES: C=CCC(C)(c1cccc(Br)c1)c1nc(-c2cc(Oc3c(F)cc4[nH]ccc4c3CCC(=O)OCC)ccc2F)n(C)n1]

Step A: To a stirred solution of ethyl 3-(6-fluoro-5-(4-fluoro-3-(imino(methylthio)methyl)phenoxy)-1H-indol-4-yl)propanoate hydroiodide (Intermediate 65, 1.6 g, 3 mmol) in pyridine (20 mL) was added 2-(3-bromophenyl)-N′,2-dimethylpent-4-enehydrazide (Intermediate 67-8, 900 mg, 3 mmol). The mixture was stirred at 85° C. for 12 hours and concentrated. The residue was re-dissolved in ethyl acetate (100 mL), washed with water (100 mL), brine, dried over magnesium sulfate and concentrated. The crude product was purified by silica gel column chromatography (eluting with 15% ethyl acetate in petroleum ether) to give the title compound (1.6 g, 82%) as a solid. MS (ESI): 649, 651 m/z [M+H]⁺, retention time: 2.29 minutes, purity: 90% (214 nm) (LC-MS method 20).

### 3-(5-(3-(3-(2-(3-Bromophenyl)pent-4-en-2-yl)-1-methyl-1H-1,2,4-triazol-5-yl)-4-fluorophenoxy)-6-fluoro-1H-indol-4-yl)propanoic acid

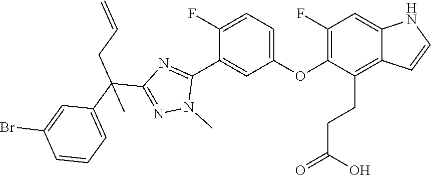 [SMILES: C=CCC(C)(c1cccc(Br)c1)c1nc(-c2cc(Oc3c(F)cc4[nH]ccc4c3CCC(=O)O)ccc2F)n(C)n1]

Step B: To a stirred solution of Step A product (1.2 g, 1.85 mmol) in 12 mL of tetrahydrofuran and 3 mL of water was added lithium hydroxide monohydrate (390 mg, 9.25 mmol). The mixture was stirred at room temperature for 2 hours, diluted with water, and acidified to pH˜1 with 2 N hydrochloric acid. The solution was extracted with ethyl acetate. The combined organic extractions were washed brine, dried over sodium sulfate, and concentrated to afford the crude title compound (1 g, crude). MS (ESI): 621, 623 m/z [M+H]⁺, retention time: 2.09 minutes, purity: 90% (214 nm) (LC-MS method 20).

### N-(Allylsulfonyl)-3-(5-(3-(3-(2-(3-bromophenyl)pent-4-en-2-yl)-1-methyl-1H-1,2,4-triazol-5-yl)-4-fluorophenoxy)-6-fluoro-1H-indol-4-yl)propanamide

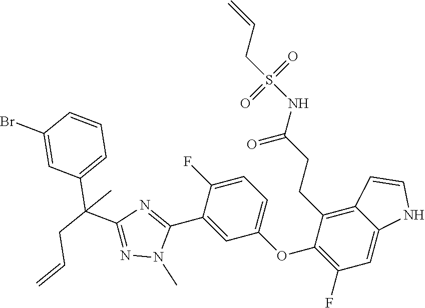 [SMILES: C=CCC(C)(c1cccc(Br)c1)c1nc(-c2cc(Oc3c(F)cc4[nH]ccc4c3CCC(=O)NS(=O)(=O)CC=C)ccc2F)n(C)n1]

Step C: To a stirred and cooled (0° C.) solution of Step B product (1.8 g, 2.9 mmol) in acetonitrile (20 mL) was added prop-2-ene-1-sulfonamide (420 mg, 3.47 mmol), N-(3-(dimethylamino)-propyl)propionimidamide hydrochloride (830 mg, 4.35 mmol), and 4-(dimethylamino)-pyridine (640 mg, 5.22 mmol). The mixture was stirred at room temperature for 12 hours, quenched with water (100 mL), and extracted with ethyl acetate (2×100 mL). The combined organic layers were washed with brine, dried over sodium sulfate, and concentrated. The residue was purified by silica gel column chromatography (eluting with 20% ethyl acetate in petroleum ether) to give the title compound (1.4 g, 70%). MS (ESI): 724, 726 m/z [M+H]⁺, retention time: 2.12 minutes, purity: 90% (214 nm) (LC-MS method 20).

### (8Z)-6-(3-Bromophenyl)-23,29-difluoro-3, 6-dimethyl-11,11-dioxo-25-oxa-116-thia-3,4,12,20,31-pentazapentacyclo[24.3.1.12,5.016,24.017,21]hentriaconta-1(30),2(31),4,8,16,18,21,23,26,28-decaen-13-one

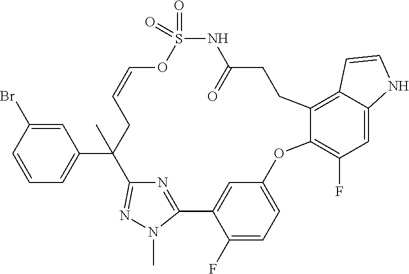 [SMILES: Cn1nc2nc1-c1cc(ccc1F)Oc1c(F)cc3[nH]ccc3c1CCC(=O)NS(=O)(=O)O/C=C\CC2(C)c1cccc(Br)c1]

Step D: To a stirred solution of Step C product (1.2 g, 1.66 mmol) in toluene (700 mL) was added Grubbs II generation catalyst (282 mg, 0.332 mmol). The mixture was stirred for 45 minutes at 65° C. and concentrated. The residue was purified by silica gel column chromatography (eluting with 25% ethyl acetate in petroleum ether) to give the title compound (700 mg, 60%) as a solid. MS (ESI): 696, 698 m/z [M+H]⁺, retention time: 2.03 minutes, purity: 90% (214 nm) (LC-MS method 20).

### Compound 114: 3-[3-(23,29-Difluoro-3,6-dimethyl-11,11,13-trioxo-25-oxa-11λ6-thia-3,4,12,20,31-pentazapentacyclo[24.3.1.12,5.016,24.017,21]hentriaconta-1(30),2(31),4,16,18,21,23,26,28-nonaen-6-yl)phenyl]-2-methyl-propanoic acid

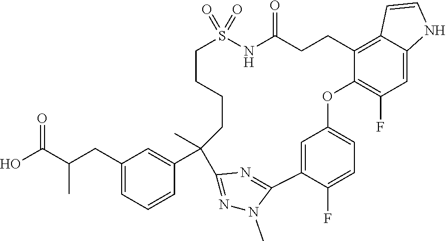 [SMILES: CC(Cc1cccc(C2(C)CCCCS(=O)(=O)NC(=O)CCc3c(c(F)cc4[nH]ccc34)Oc3ccc(F)c(c3)-c3nc2nn3C)c1)C(=O)O]

### Compound 115: 23,29-Difluoro-3,6-dimethyl-11,11-dioxo-6-phenyl-25-oxa-11A6-thia-3,4,12,20,31-pentazapentacyclo[24.3.1.12,5.016,24.017,21]hentriaconta-1(30),2(31),4,16,18,21,23,26,28-nonaen-13-one

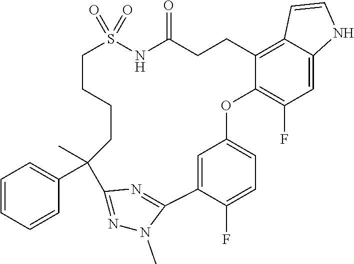 [SMILES: Cn1nc2nc1-c1cc(ccc1F)Oc1c(F)cc3[nH]ccc3c1CCC(=O)NS(=O)(=O)CCCCC2(C)c1ccccc1]

Step E: Exchanging (8Z)-6-(3-Bromophenyl)-23,29-difluoro-3,6,11,11,12-pentamethyl-14,25-dioxa-3,4,12,20,31-pentazapentacyclo [24.3.1.12,5.016,24.017,21]hentriaconta-1(30),2(31),4,8,16,18,21,23,26,28-decaen-13-one (Step F product of Example 103) with (8Z)-6-(3-Bromophenyl)-23,29-difluoro-3,6-dimethyl-11,11-dioxo-25-oxa-11λ6-thia-3,4,12,20,31-pentazapentacyclo[24.3.1.12,5.016,24.017,21]hentriaconta-1(30),2(31),4,8,16,18,21,23,26,28-decaen-13-one (Step D product of this example, 550 mg, 0.8 mmol), the reaction procedure sequence (Steps G, H and I, in this order) described for Example 103 was used to prepare the title compounds. 115 (8 mg, white solid), the de-bromination product originated from corresponding Step G, was separated by Prep-HPLC after hydrogenation, the corresponding Step H. 114 (12 mg) was obtained as a white solid, after corresponding Step I of Example 103.

Compound 115: MS (ESI): 620 m/z [M+H]⁺, retention time: 1.87 minutes, purity: >99% (214 nm) (LC-MS method 20). ¹H NMR (500 MHz, CD₃OD) δ 7.37-7.36 (m, 1H), 7.28-7.20 (m, 6H), 7.20-7.11 (m, 2H), 6.98-6.96 (m, 1H), 6.54 (d, J=3.5 Hz, 1H), 3.89 (d, J=2.5 Hz, 3H), 3.51-3.44 (m, 1H), 3.20-3.05 (m, 4H), 2.75-2.63 (m, 1H), 2.60-2.51 (m, 1H), 2.40-2.34 (m, 1H), 2.20-2.15 (m, 1H), 1.92-1.82 (m, 1H), 1.78-1.58 (m, 5H) ppm.

Compound 114: MS (ESI): 706 m/z [M+H]⁺, retention time: 1.76 minutes, purity: 97% (214 nm) (LC-MS method 20). ¹H NMR (500 MHz, CD₃OD) δ 7.35-7.33 (m, 1H), 7.28-7.15 (m, 4H), 7.12-7.10 (m, 1H), 7.07-6.94 (m, 3H), 6.54 (d, J=3.0 Hz, 1H), 3.89 (d, J=2.5 Hz, 3H), 3.52-3.42 (m, 1H), 3.20-3.14 (m, 1H), 3.12-3.05 (m, 2H), 2.98-2.88 (m, 1H), 2.67-2.48 (m, 4H), 2.36-2.30 (m, 1H), 2.19-2.08 (m, 1H), 1.91-1.82 (m, 1H), 1.78-1.58 (m, 6H), 1.09-1.07 (m, 3H) ppm.

### Example 116. Compound 116. (2S)-3-[3-(22,28-difluoro-3,6,10,10-tetramethyl-12,12-dioxo-24-oxa-12λ6-thia-3,4,11,19,30-pentazapentacyclo[23.3.1.12,5.015,23.016,20]triaconta-1(29),2(30),4,15,17,20,22,25,27-nonaen-6-yl)phenyl]-2-methyl-propanoic acid

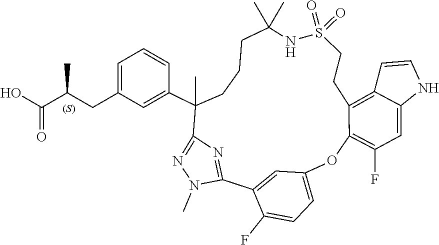

### Methyl (2S)-3-(3-(2-(5-(5-((4-bromo-6-fluoro-1H-indol-5-yl)oxy)-2-fluorophenyl)-1-methyl-1H-1,2,4-triazol-3-yl)-6-methyl-6-nitroheptan-2-yl)phenyl)-2-methylpropanoate

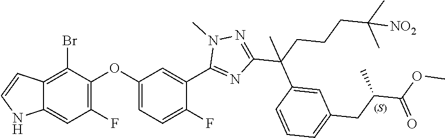

Step A: To a stirred solution of methyl (2S)-3-(3-(2,6-dimethyl-1-(2-methylhydrazineyl)-6-nitro-1-oxoheptan-2-yl)phenyl)-2-methylpropanoate (Intermediate 67-9, 3.29 g, 8.07 mmol) in pyridine (40 mL) was added methyl 5-((4-bromo-6-fluoro-1H-indol-5-yl)oxy)-2-fluorobenzimidothioate hydroiodide (Intermediate 65-3, 4.66 g, 8.88 mmol) and magnesium sulphate (2 g). The reaction mixture was stirred at 85° C. for 3 hours, cooled to room temperature, and diluted with ethyl acetate (200 mL). The solution was washed with brine, dried over sodium sulfate, and concentrated. The residue was purified by flash chromatography (40 g silica gel column, eluting with 0-70% ethyl acetate in petroleum ether) to give the title compound (3.86 g, 65%) as solid. MS (ESI): 738, 740 m/z [M+H]⁺, retention time: 2.11 minutes, purity: 97% (214 nm) (LC-MS method 25).

### Methyl (2S)-3-(3-(2-(5-(5-((4-bromo-6-fluoro-I-tosyl-1H-indol-5-yl)oxy)-2-fluorophenyl)-1-methyl-1H-1,2,4-triazol-3-yl)-6-methyl-6-nitroheptan-2-yl)phenyl)-2-methylpropanoate

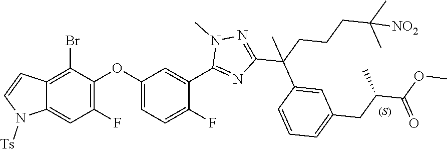

Step B: To a stirred solution of Step A product (3.86 g, 5.23 mmol) in acetonitrile (40 mL) was added 1-tosyl-1H-imidazole (1.39 g, 6.28 mmol), 1,8-diazabicyclo[5.4.0]undec-7-ene (0.47 mL, 3.14 mmol). The mixture was stirred at room temperature overnight, diluted with water (30 mL), and extracted with ethyl acetate (3×50 mL). The combined organic extracts were washed with brine, dried over sodium sulfate, and concentrated. The residue was purified by automated flash chromatography (20 g silica gel column, eluting with 0-70% ethyl acetate in petroleum ether) to give the title compound (4 g, 85%) as a solid. MS (ESI): 892, 894 m/z [M+H]⁺, retention time: 1.65 minutes, purity: 99% (214 nm) (LC-MS method 26).

### Methyl (2S)-3-(3-(6-amino-2-(5-(5-((4-bromo-6-fluoro-l-tosyl-1H-indol-5-yl)oxy)-2-fluorophenyl)-1-methyl-1H-1,2,4-triazol-3-yl)-6-methylheptan-2-yl)phenyl)-2-methylpropanoate

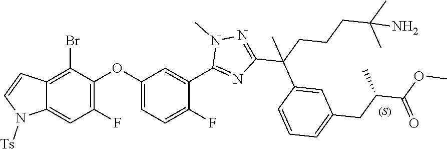

Step C: To a stirred solution of Step B product (4 g, 4.48 mmol) in ethanol/water (V=5:1) (160 mL) was added iron powder (2 g, 36 mmol) and ammonium chloride (2.41 g, 44. 8 mmol). The mixture was refluxed overnight, cooled to room temperature, and filtered through a pad of Celite. The filter cake was washed with ethyl acetate. The combined filtrate was concentrated. The residue was dissolved in ethyl acetate (150 mL), washed with brine, dried over sodium sulphate, and concentrated. The crude product was purified by flash chromatography (40 g silica gel column, eluting with 0-10% methanol in dichloromethane) to give the title compound (3.28 g, 85%) as a solid. MS (ESI): 862, 864 m/z [M+H]⁺, retention time: 1.76 minutes, purity: 99% (214 nm) (LC-MS method 25).

### Methyl (2S)-3-(3-(2-(5-(5-((4-bromo-6-fluoro-l-tosyl-1H-indol-5-yl)oxy)-2-fluorophenyl)-1-methyl-1H-1,2,4-triazol-3-yl)-6-methyl-6-(vinylsulfonamido)heptan-2-yl)phenyl)-2-methylpropanoate

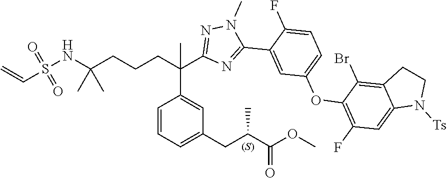

Step D: To a stirred solution of Step C product (3.28 g, 3.8 mmol) in N,N-dimethylformamide (28 mL), was added 2-chloroethane-1-sulfonyl chloride (929 mg, 5.7 mmol), 4-dimethylaminopyridine (28 mg, 0.11 mmol) and N,N-diisopropylethylamine (1.36 mL, 7.6 mmol). The mixture was stirred at 70° C. overnight, cooled to room temperature, and quenched with water (30 mL). The solution was extracted with ethyl acetate (3×50 mL). The combined organic extracts were washed with brine, dried over sodium sulphate, and concentrated. The residue was purified by flash chromatography (20 g silica gel column, eluting with 0-50% ethyl acetate in petroleum ether) to give the title compound (1.2 g, 33%) as solid. MS (ESI): 952, 954 m/z [M+H]⁺, retention time: 2.19 minutes, purity: 99% (214 nm) (LC-MS method 25).

### Methyl (2S)-3-[3-[(13E)-22,28-difluoro-3,6,10,10-tetramethyl-12,12-dioxo-]9-(p-tolylsulfonyl)-24-oxa-12A6-thia-3,4,11,19,30-pentazapentacyclo[23.3.1.12,5.015,23.016,20]triaconta-1(29),2(30),4,13,15,17,20,22,25,27-decaen-6-yl]phenyl]-2-methyl-propanoate

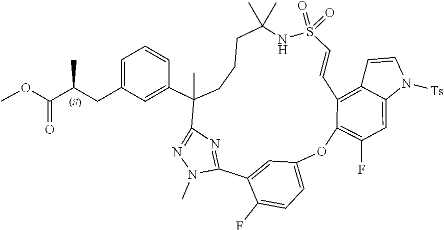

Step E: To a stirred solution of Step D product (1.4 g, 1.26 mmol) in dry toluene (720 mL) was added triethylamine (1.76 mL, 12.6 mmol) and bis(tri-tertbutylphosphine)palladium(0) (128 mg, 0.25 mmol). The mixture was stirred at 100° C. for 2 hours and concentrated. The residue was purified with automated flash chromatography (40 g silica gel column, eluting with 0-70% ethyl acetate in petroleum ether) to afford the title compound (710 mg, 65%) as a solid. MS (ESI): 872 m/z [M+H]⁺, retention time: 1.58 minutes, purity: 99% (214 nm) (LC-MS method 26).

### Methyl (2S)-3-[3-[(13E)-22,28-difluoro-3,6,10,10-tetramethyl-12,12-dioxo-24-oxa-12A6-thia-3,4,11,19,30-pentazapentacyclo[23.3.1.12,5.015,23.016,20]triaconta-1(29),2(30),4,13,15,17,20,22,25,27-decaen-6-yl]phenyl]-2-methyl-propanoate

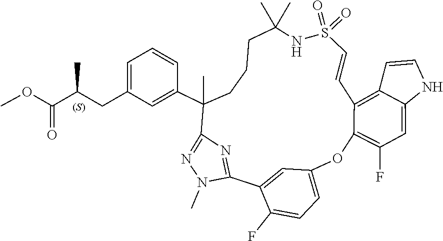

Step F: To a stirred solution of Step E product (310 mg, 0.36 mmol) in methanol (20 mL) was added potassium carbonate (98 mg, 0.7 mmol). The mixture was stirred at 60° C. for 1 hour and concentrated. The residue was diluted with water (30 mL), extracted with ethyl acetate (3×20 mL). The combined organic extracts were washed with brine, dried over sodium sulphate, and concentrated. The crude product was purified by automated flash chromatography (20 g silica gel column, eluting with 0-70% ethyl acetate in petroleum ether) to afford the title compound (216 mg, 85%) as a solid. MS (ESI): 718 m/z [M+H]⁺, retention time: 2.00 minutes, purity: 98% (214 nm) (LC-MS method 25).

### Methyl (2S)-3-[3-(22,28-difluoro-3,6,10,10-tetramethyl-12,12-dioxo-24-oxa-12A6-thia-3,4,11,19,30-pentazapentacyclo[23.3.1.12,5.015,23.016,20]triaconta-1(29),2(30),4,15,17,20,22,25,27-nonaen-6-yl)phenyl]-2-methyl-propanoate

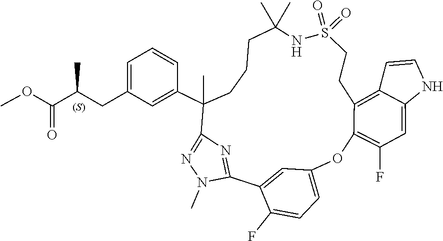

Step G: To a stirred solution of Step G product (216 mg, 0.3 mmol) in ethanol (20 mL) was added palladium on carbon (10%, 50% wet) (100 mg). The mixture was stirred at 50° C. for 2 hours under hydrogen balloon, cooled to room temperature and filtered through a pad of Celite. The filter cake was washed with ethyl acetate (50 mL). The combined filtrate was concentrated. The residue was purified with automated flash chromatography (10 g silica gel column, eluting with 0-70% ethyl acetate in petroleum ether) to afford the title compound (200 mg, 92%) as a solid. MS (ESI): 720 m/z [M+H]⁺, retention time: 1.97 minutes, purity: 97% (214 nm) (LC-MS method 25).

### Compound 116: (2S)-3-[3-(22,28-difluoro-3,6,10,10-tetramethyl-12,12-dioxo-24-oxa-126-thia-3,4,11,19,30-pentazapentacyclo[23.3.1.12,5.015,23.016,20]triaconta-1(29),2(30),4,15,17,20,22,25,27-nonaen-6-yl)phenyl]-2-methyl-propanoic acid

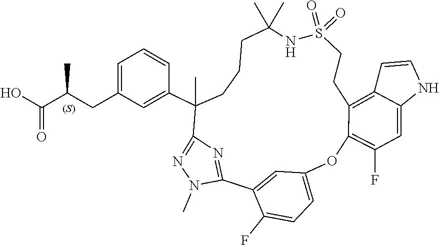

Step H: To a stirred solution of Step G product (20 mg, 0.027 mmol) in tetrahydrofuran/water/methanol (6 mL/2 mL/2 mL) was added lithium hydroxide monohydrate (5.6 mg, 0.14 mmol). The reaction was stirred at room temperature for 1.5 hours and acidified with 1N hydrochloric acid to pH˜4. The solution was diluted with ethyl acetate (50 mL), washed with brine, dried over sodium sulphate, and concentrated. The residue was purified by prep-HPLC to give the title compound (4 mg, 20%) as a white solid. MS (ESI): 706 m/z [M+H]⁺, retention time: 1.61 minutes, purity: 97% (214 nm) (LC-MS method 16). ¹H NMR (400 MHz, CD₃OD) δ 7.52-7.46 (m, 1H), 7.38 (t, J=9.2 Hz, 1H), 7.34 (d, J=3.2 Hz, 1H), 7.20 (d, J=10.4 Hz, 1H), 7.09-7.04 (m, 1H), 6.97-6.89 (m, 3H), 6.7-6.67 (m, 1H), 6.54 (d, J=3.2 Hz, 1H), 3.74 (s, 3H), 3.36-3.31 (m, 2H), 3.22-3.12 (m, 2H), 2.90-2.82 (m, 1H), 2.54-2.47 (m, 2H), 2.09-2.03 (m, 1H), 1.79-1.76 (m, 1H), 1.66 (s, 3H), 1.56-1.50 (m, 1H), 1.37-1.31 (m, 2H), 1.24 (s, 3H), 1.21 (s, 3H), 1.15-1.11 (m, 1H), 0.99-0.94 (m, 3H) ppm.

### Example 117. Compound 117A and 117B. Diastereomers 1 and 2 of (2S)-3-[3-[22,28-difluoro-3,6,10,10,11-pentamethyl-12,12-dioxo-24-oxa-12%6-thia-3,4,11,19,30-pentazapentacyclo[23.3.1.12,5.015,23.016,20]triaconta-1(29),2(30),4,15,17,20,22,25,27-nonaen-6-yl]phenyl]-2-methyl-propanoic acid

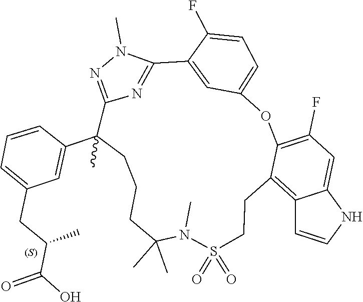

### Methyl (2S)-3-[3-[(13E)-22,28-difluoro-3,6,10,10,11-pentamethyl-12,12-dioxo-19-(p-tolylsulfonyl)-24-oxa-12A6-thia-3,4,11,19,30-pentazapentacyclo[23.3.1.12,5.015,23.016,20]-triaconta-1(29),2(30),4,13,15,17,20,22,25,27-decaen-6-yl]phenyl]-2-methyl-propanoate

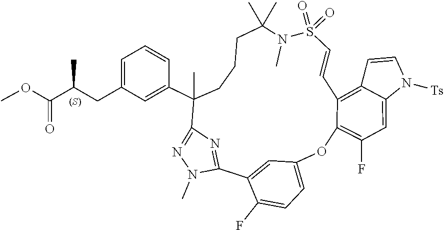

Step A: To a stirred solution of methyl (2S)-3-[3-[(13E)-22,28-difluoro-3,6,10,10-tetramethyl-12,12-dioxo-19-(p-tolylsulfonyl)-24-oxa-12λ6-thia-3,4,11,19,30-pentazapentacyclo[23.3.1.12,5.015,23.016,20]triaconta-1(29),2(30),4,13,15,17,20,22,25,27-decaen-6-yl]phenyl]-2-methyl-propanoate (Step E product of Example 116, 0.402 g, 0.46 mmol) in tetrahydrofuran (10 mL) was added sodium hydride (60% in mineral oil, 37 mg, 0.92 mmol). The mixture was stirred at room temperature for 30 minutes, treated with iodomethane (12.8 uL, 0.2 mmol) and stirred at room temperature for an additional 2 hours. The reaction mixture was concentrated. The residue was purified by automated flash chromatography (4 g silica gel column, eluting with 0-70% ethyl acetate in petroleum ether) to afford the title compound (245 mg, 60%) as a solid. MS (ESI): 886 m/z [M+H]⁺, retention time: 2.28 minutes, purity: >99% (214 nm) (LC-MS method 25).

### Compound 117A and 117B. Diastereomers 1 and 2 of (2S)-3-[3-[22,28-difluoro-3,6,10,10,11-pentamethyl-12,12-dioxo-24-oxa-12 6-thia-3,4,11,19,30-pentazapentacyclo[23.3.1.12,5.015,23.016,20]triaconta-1(29),2(30),4,15,17,20,22,25,27-nonaen-6-yl]phenyl]-2-methyl-propanoic acid

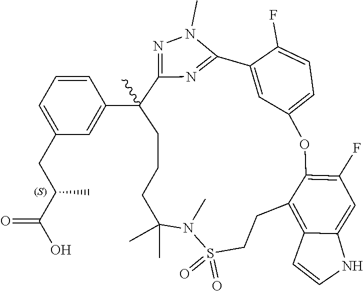

Exchanging methyl (2S)-3-[3-[(13E)-22,28-difluoro-3,6,10,10-tetramethyl-12,12-dioxo-19-(p-tolylsulfonyl)-24-oxa-12λ6-thia-3,4,11,19,30-pentazapentacyclo [23.3.1.12,5.015,23.016,20]-triaconta-1(29),2(30),4,13,15,17,20,22,25,27-decaen-6-yl]phenyl]-2-methyl-propanoate (Step E product of Example 116) with methyl (2S)-3-[3-[(13E)-22,28-difluoro-3,6,10,10,11-pentamethyl-12,12-dioxo-19-(p-tolylsulfonyl)-24-oxa-12λ6-thia-3,4,11,19,30-pentazapentacyclo [23.3.1.12,5.015,23.016,20]-triaconta-1(29),2(30),4,13,15,17,20,22,25,27-decaen-6-yl]phenyl]-2-methyl-propanoate (Step A product of this Example, 245 mg, 0.27 mmol), the reaction procedure sequence (Steps F, G, H, in this order) described for Example 116 was used to prepare the title compounds. The methyl ester (144 mg), obtained from corresponding Step G of Example 116, was subjected to chiral Prep-HPLC separation under the following conditions: Column: CHIRALPAK, 5.0 cm I.D.×25 cm L, 10 μm; Sample solution: 1.7 mg/mL Mobile phase; Injection: 30 mL; Mobile phase: Hexane/isopropyl alcohol=75/25 (V/V) Flow rate: 60 mL/minute; Wavelength: UV 214 nm; Temperature: 38° C. The first eluent (48 mg, 33%), Diastereomer 1, was further hydrolyzed to 117A (40.7 mg, 86, white solid); The second eluent (44 mg, 31%), Diastereomer 2, was further hydrolyzed to 117B (35.2 mg, 82%, white solid), following conditions described in Step G of Example 116.

Compound 117A: MS (ESI): 720 m/z [M+H]⁺, retention time: 1.93 minutes, purity: 99% (214 nm) (LC-MS method 25). ¹H NMR (400 MHz, CD₃OD) δ 7.52-7.47 (m, 1H), 7.38 (t, J=9.2 Hz, 1H), 7.32 (d, J=3.2 Hz, 1H), 7.19 (d, J=10.4 Hz, 1H), 6.96-6.80 (m, 4H), 6.72-6.69 (m, 1H), 6.50 (d, J=2.8 Hz, 1H), 3.78 (d, J=2.0 Hz, 3H), 3.37-3.12 (m, 4H), 2.80 (dd, J=13.2, 6.4 Hz, 1H), 2.61 (s, 3H), 2.52-2.38 (m, 2H), 2.08-2.02 (m, 1H), 1.79-1.73 m, 2H), 1.64 (s, 3H), 1.46-1.39 (m, 2H), 1.31-1.22 (m, 6H), 1.05-1.00 (m, 1H), 0.95 (d, J=7.2 Hz, 3H) ppm.

Compound 117B: MS (ESI): 720 m/z [M+H]⁺, retention time: 1.94 minutes, purity: 99% (214 nm) (LC-MS method 25). ¹H NMR (400 MHz, CD₃OD) δ 7.52-7.47 (m, 1H), 7.38 (t, J=9.2 Hz, 1H), 7.32 (d, J=3.2 Hz, 1H), 7.19 (d, J=10.4 Hz, 1H), 6.96-6.79 (m, 4H), 6.71-6.69 (m, 1H), 6.50 (d, J=2.8 Hz, 1H), 3.78 (d, J=2.0 Hz, 3H), 3.37-3.13 (m, 4H), 2.73-2.69 (m, 1H), 2.61 (s, 3H), 2.51-2.41 (m, 2H), 2.07-2.01 (m, 1H), 1.80-1.75 (m, 2H), 1.64 (s, 3H), 1.45-1.39 (m, 2H), 1.30-1.21 (m, 6H), 1.02-0.95 (m, 1H), 0.89 (d, J=6.8 Hz, 3H) ppm.

### Example 118. Compound 118. (2S)-3-[3-[(6R)-23-fluoro-3,6,10,10,12-pentamethyl-13-oxo-25-oxa-3,4,12,20,29,31-hexazapentacyclo[24.3.1.12,5.016,24.017,21]hentriaconta-1(30),2(31),4,16,18,21,23,26,28-nonaen-6-yl]phenyl]-2-methyl-propanoic acid

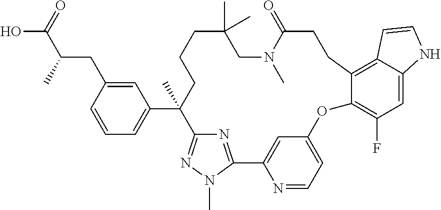 [SMILES: C[C@@H](Cc1cccc([C@@]2(C)CCCC(C)(C)CN(C)C(=O)CCc3c(c(F)cc4[nH]ccc34)Oc3ccnc(c3)-c3nc2nn3C)c1)C(=O)O]

### Ethyl (R,E)-3-(5-((2-(3-(7-(((benzyloxy)carbonyl)(methyl)amino)-2-(3-iodophenyl)-6,6-dimethylheptan-2-yl)-1-methyl-1H-1,2,4-triazol-5-yl)pyridin-4-yl)oxy)-6-fluoro-1H-indol-4-yl)acrylate

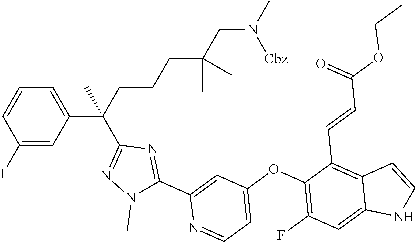 [SMILES: CCOC(=O)/C=C/c1c(Oc2ccnc(-c3nc([C@](C)(CCCC(C)(C)CN(C)C(=O)OCc4ccccc4)c4cccc(I)c4)nn3C)c2)c(F)cc2[nH]ccc12]

Step A: To a stirred solution of benzyl (R)-(6-(3-iodophenyl)-2,2,6-trimethyl-7-(2-methylhydrazineyl)-7-oxoheptyl)(methyl)carbamate (Intermediate 67-10, 1094 mg, 1.93 mmol) in pyridine (15 mL) were added ethyl (E)-3-(6-fluoro-5-((2-(imino(methylthio)methyl)pyridin-4-yl)oxy)-1H-indol-4-yl)acrylate (Intermediate 75, 850 mg, 2.13 mmol) and magnesium sulfate (1164 mg, 9.67 mmol). The reaction was stirred at room temperature for 16 hours, then at 60° C. for 5 hours. The mixture was diluted with ethyl acetate (40 mL), washed with 1N hydrochloric acid, and brine. The separated organic layer was dried over sodium sulfate, filtered, and concentrated. The residue was purified by automated flash chromatography (40 g silica gel column, eluting with 0-5% methanol in dichloromethane) to give the title compound (1.2 g, 0.98 mmol, 46%) as a yellow solid. LC-MS: MS (ESI): 899 m/z [M+H]⁺, retention time: 1.61 minutes, purity: 73% (254 nm) (LC-MS Method 003).

### Ethyl (R,E)-3-(6-fluoro-5-((2-(3-(2-(3-iodophenyl)-6,6-dimethyl-7-(methylamino)heptan-2-yl)-1-methyl-1H-1,2,4-triazol-5-yl)pyridin-4-yl)oxy)-1H-indol-4-yl)acrylate

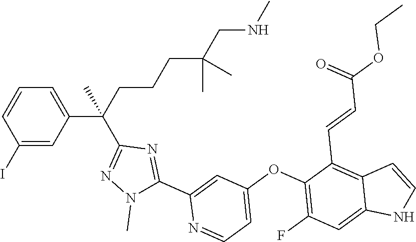 [SMILES: CCOC(=O)/C=C/c1c(Oc2ccnc(-c3nc([C@](C)(CCCC(C)(C)CNC)c4cccc(I)c4)nn3C)c2)c(F)cc2[nH]ccc12]

Step B: To a stirred and cooled (0° C.) solution of Step A product (1.2 g, 1.28 mmol) in dichloromethane (10 mL) was added iodotrimethylsilane (0.70 mL, 5.13 mmol). The reaction mixture was stirred at 0° C. for 1 hours and concentrated. The residue was triturated with petroleum ether (20 mL) to give the title compound (850 mg, 1.11 mmol, 87%) as a brown solid. LC-MS: MS (ESI): 765 m/z [M+H]⁺, retention time: 1.28 minutes, purity: >99% (254 nm) (LC-MS Method 028).

### (6R,14E)-23-Fluoro-6-(3-iodophenyl)-3,6,10,10,12-pentamethyl-25-oxa-3,4,12,20,29,31-hexazapentacyclo[24.3.1.12,5.016,24.017,21]hentriaconta-1(30),2(31),4,14,16,18,21,23,26,28-decaen-13-one

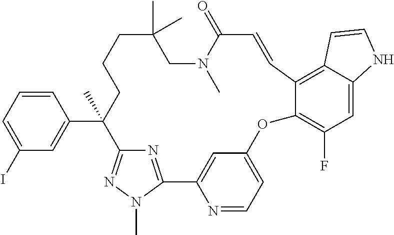 [SMILES: CN1CC(C)(C)CCC[C@](C)(c2cccc(I)c2)c2nc(n(C)n2)-c2cc(ccn2)Oc2c(F)cc3[nH]ccc3c2/C=C/C1=O]

Step C: Exchanging Methyl (E)-3-(5-(3-(5-(5-cyano-1-(3-iodophenyl)pentyl)-1H-imidazol-2-yl)-4-fluorophenoxy)-6-fluoro-1H-indol-4-yl)acrylate (Step A product of Example 1) with Step A product (850 mg, 1.11 mmol) of this example, The reaction procedure sequence (Steps B and E, in this order) described for Example 1 was used to prepare the title compound (320 mg, 0.41 mmol) as a yellow oil. LC-MS: MS (ESI): 719 m/z [M+H]⁺, retention time: 2.52 minutes, purity: 93% (214 nm) (LC-MS Method 028).

### Methyl (2S)-3-[3-[(6R,14E)-23-fluoro-3,6,10,10,12-pentamethyl-13-oxo-25-oxa-3,4,12,20,29,31-hexazapentacyclo[24.3.1.12,5.016,24.017,21]hentriaconta-1(30),2(31),4,14,16,18,21,23,26,28-decaen-6-yl]phenyl]-2-methyl-propanoate

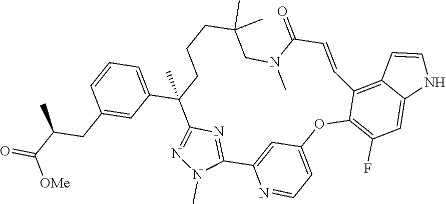 [SMILES: COC(=O)[C@@H](C)Cc1cccc([C@@]2(C)CCCC(C)(C)CN(C)C(=O)/C=C/c3c(c(F)cc4[nH]ccc34)Oc3ccnc(c3)-c3nc2nn3C)c1]

Step D: To a suspension of zinc (6.00 eq, 55 mg, 0.835 mmol) in N, N-dimethylformamide (2 mL) was added iodine (3.5 mg, 0.0139 mmol). The reaction was stirred for 30 minutes at room temperature, then treated with methyl (2R)-3-iodo-2-methyl-propanoate (95 mg, 0.417 mmol). The mixture was stirred for 40 minutes, followed by addition of Step C product (100 mg, 0.139 mmol), tris(dibenzylideneacetone)dipalladium (13 mg, 0.0139 mmol) and 2-dicyclohexylphosphino-2′,6′-dimethoxybiphenyl (11 mg, 0.0278 mmol). The reaction mixture was stirred at room temperature for 16 hours, quenched with water (15 mL), and extracted with ethyl acetate (2×10 mL). The combined organic phase was washed with brine (10 mL), dried over sodium sulfate, filtered, and concentrated. The residue was purified by silica gel column chromatography (25 g silica gel column, eluting with 0-40% ethyl acetate in petroleum ether) to give the title compound (85 mg, 0.110 mmol, 79%) as a pale-yellow oil. LC-MS: MS (ESI): 693 m/z [M+H]⁺, retention time: 1.91 minutes, purity: 90% (254 nm) (LC-MS Method 3).

### Methyl (2S)-3-[3-[(6R)-23-fluoro-3,6,10,10,12-pentamethyl-13-oxo-25-oxa-3,4,12,20,29,31-hexazapentacyclo[24.3.1.12,5.016,24.017,21]hentriaconta-1(30),2(31),4,16,18,21,23,26,28-nonaen-6-yl]phenyl]-2-methyl-propanoate

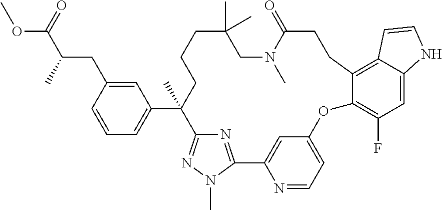 [SMILES: COC(=O)[C@@H](C)Cc1cccc([C@@]2(C)CCCC(C)(C)CN(C)C(=O)CCc3c(c(F)cc4[nH]ccc34)Oc3ccnc(c3)-c3nc2nn3C)c1]

Step E: To a stirred solution of Step D product (85 mg, 0.123 mmol) in ethanol (10 mL) was added palladium on carbon (20 mg, 0.188 mmol). The mixture was stirred at 50° C. for 16 hours under hydrogen atmosphere, then filtered through a pad of Celite. The filter cake was washed with ethanol (10 mL). The filtrate was concentrated to give the title compound (72 mg, 0.087 mmol, 71%) as a yellow oil. LC-MS: MS (ESI): 695 m/z [M+H]⁺, retention time: 1.68 minutes, purity: 84% (214 nm) (LC-MS Method 28).

### Compound 118. (2S)-3-[3-[(6R)-23-Fluoro-3,6,10,10,12-pentamethyl-13-oxo-25-oxa-3,4,12,20,29,31-hexazapentacyclo[24.3.1.12,5.016,24.017,21]hentriaconta-1(30),2(31),4,16,18,21,23,26,28-nonaen-6-yl]phenyl]-2-methyl-propanoic acid

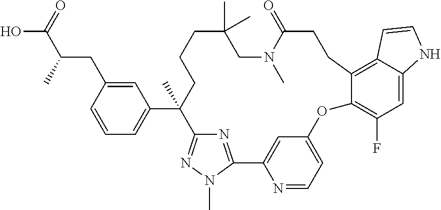 [SMILES: C[C@@H](Cc1cccc([C@@]2(C)CCCC(C)(C)CN(C)C(=O)CCc3c(c(F)cc4[nH]ccc34)Oc3ccnc(c3)-c3nc2nn3C)c1)C(=O)O]

Step F: To a stirred solution of Step E product (55 mg, 0.079 mmol) in tetrahydrofuran (3 mL), methanol (1 mL) and water (1 mL) was added lithium hydroxide monohydrate (33 mg, 0.79 mmol). The mixture was stirred at room temperature for 2 hours, then acidified to pH 5-6 with hydrochloric acid (1N) and extracted with dichloromethane (3×5 mL). The combined organic layer was washed with brine, dried over sodium sulfate, filtered, and concentrated. The residue was purified by prep-HPLC (high pH, ammonium bicarbonate) to give the title compound (21 mg, 0.031 mmol, 39%) as a white solid. LC-MS: MS (ESI): 681 m/z [M+H]⁺, retention time: 1.68 minutes, purity: >99% (214 nm) (LC-MS Method 4). ¹H NMR (400 MHz, CD₃OD) δ 8.61 (d, J=5.6 Hz, 0.5H), 8.52 (d, J=5.6 Hz, 0.5H), 8.05 (d, J=2.4 Hz, 0.5H), 7.54 (d, J=2.4 Hz, 0.5H), 7.31-7.28 (m, 1H), 7.24-7.22 (m, 1H), 7.19-7.09 (m, 2.5H), 7.01-6.94 (m, 2H), 6.76-6.74 (m, 0.5H), 6.61 (d, J=3.2 Hz, 0.5H), 6.44 (d, J=3.2 Hz, 0.5H), 4.34-4.32 (m, 3H), 3.58 (d, J=13.6 Hz, 0.5H), 3.18-3.10 (m, 1.5H), 3.07 (s, 1.5H), 3.01-2.88 (m, 2H), 2.83 (s, 1.5H), 2.79-2.75 (m, 1.5H), 2.68-2.55 (m, 4H), 2.51-2.32 (m, 1H), 2.12-2.06 (m, 1H), 1.83-1.72 (m, 0.5H), 1.68 (s, 1.5H), 1.60 (s, 1.5H) 1.47-1.34 (m, 2H), 1.15-1.08 (s, 1.5H), 1.02-0.98 (m, 1.5H), 0.95-0.83 (m, 1H), 0.76-0.72 (m, 4.5H), 0.72 (s, 1.5H) ppm.

### Example 119. Compound 119. (2R)-3-[3-[(6R)-23-fluoro-3,6,10,10,12-pentamethyl-13-oxo-25-oxa-3,4,12,20,29,31-hexazapentacyclo[24.3.1.12,5.016,24.017,21]hentriaconta-1(30),2(31),4,16,18,21,23,26,28-nonaen-6-yl]phenyl]-2-methyl-propanoic acid

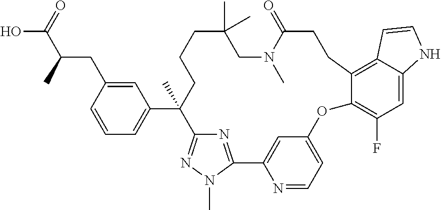 [SMILES: C[C@H](Cc1cccc([C@@]2(C)CCCC(C)(C)CN(C)C(=O)CCc3c(c(F)cc4[nH]ccc34)Oc3ccnc(c3)-c3nc2nn3C)c1)C(=O)O]

Exchanging methyl (2R)-3-iodo-2-methyl-propanoate with methyl (2S)-3-iodo-2-methyl-propanoate (81 mg, 0.355 mmol), the reaction procedure sequence (Steps D to F) described for Example 118 was used to prepare the title compound (14 mg, 0.021 mmol) as a white solid. LC-MS: MS (ESI): 681 m/z [M+H]⁺, retention time: 1.82 minutes, purity: >99% (214 nm) (LC-MS Method 4). ¹H NMR (400 MHz, CD₃OD) δ 8.61 (d, J=5.6 Hz, 0.5H), 8.52 (d, J=5.6 Hz, 0.5H), 8.04 (d, J=2.4 Hz, 0.5H), 7.54 (d, J=2.4 Hz, 0.5H), 7.31-7.28 (m, 1H), 7.24-7.22 (m, 1H), 7.20-7.08 (m, 2.5H), 7.01-6.94 (m, 2H), 6.77-6.74 (m, 0.5H), 6.61 (d, J=3.2 Hz, 0.5H), 6.53 (m, 0.5H), 4.34-4.32 (m, 3H), 3.58 (d, J=13.6 Hz, 0.5H), 3.18-3.10 (m, 1.5H), 3.07 (s, 1.5H), 3.00-2.87 (m, 2H), 2.81-2.75 (m, 3H), 2.67-2.60 (m, 1H), 2.59-2.54 (m, 2H), 2.49-2.32 (m, 1H), 2.14-2.06 (m, 1H), 1.83-1.95 (m, 0.5H), 1.68 (s, 1.5H), 1.60 (s, 1.5H), 1.48-1.32 (m, 2H), 1.18-1.00 (m, 4H), 0.96-0.85 (m, 1H), 0.75-0.73 (s, 4.5H), 0.72 (s, 1.5H) ppm.

### Example 120. Compound 120. 3-[3-[(6R)-23-fluoro-3,6,10,10,12-pentamethyl-13-oxo-25-oxa-3,4,12,20,29,31-hexazapentacyclo[24.3.1.12,5.016,24.017,21]hentriaconta-1(30),2(31),4,16,18,21,23,26,28-nonaen-6-yl]phenyl]-2,2-dimethyl-propanoic acid

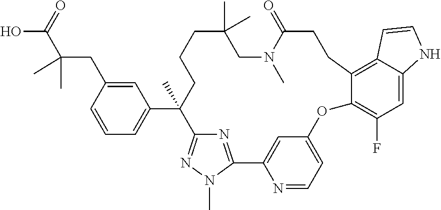 [SMILES: CN1CC(C)(C)CCC[C@](C)(c2cccc(CC(C)(C)C(=O)O)c2)c2nc(n(C)n2)-c2cc(ccn2)Oc2c(F)cc3[nH]ccc3c2CCC1=O]

Exchanging methyl (2R)-3-iodo-2-methyl-propanoate with methyl 3-iodo-2,2-dimethyl-propanoate (101 mg, 0.42 mmol), the reaction procedure sequence (Steps D to F) described for Example 118 was used to prepare the title compound (23 mg, 0.0325 mmol) as a white solid. LC-MS: MS (ESI): 695 m/z [M+H]⁺, retention time: 1.75 minutes, purity: >99% (214 nm) (LC-MS Method 4). ¹H NMR (400 MHz, CD₃OD) δ 8.61 (d, J=4.8 Hz, 0.5H), 8.51 (d, J=4.8 Hz, 0.5H), 8.03 (d, J=2.4 Hz, 0.5H), 8.03 (d, J=2.4 Hz, 0.5H), 7.31-7.28 (m, 1H), 7.25-7.20 (m, 1H), 7.17-7.07 (m, 2.5H), 6.98-6.91 (m, 2H), 6.77-6.76 (m, 0.5H), 6.61 (d, J=3.2 Hz, 0.5H), 6.44 (d, J=3.2 Hz, 0.5H), 4.33-4.31 (s, 3H), 3.55 (d, J=10.8 Hz, 0.5H), 3.17-3.11 (m, 1.5H), 3.06 (s, 1.5H), 2.98-2.91 (m, 1H), 2.82-2.69 (m, 3H), 2.59-2.56 (m, 1H), 2.50-2.40 (m, 0.5H), 2.38-2.30 (m, 0.5H), 2.12-2.05 (m, 1H), 1.80-1.75 (m, 0.5H), 1.68 (s, 1.5H), 1.60 (s, 1.5H), 1.47-1.32 (m, 2H), 1.17-1.09 (m, 4H), 1.02 (s, 3H), 0.99-0.84 (m, 1H), 0.75-0.72 (m, 4.5H), 0.56 (s, 1.5H) ppm.

### Example 121. Compound 121. (2S)-3-[3-[(6R)-23-fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]-2-methyl-propanoic acid

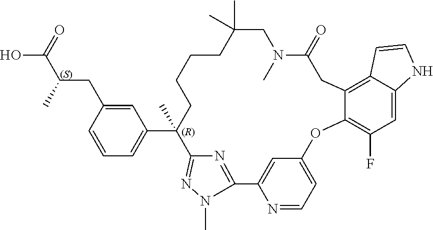

Exchanging benzyl (R)-(6-(3-iodophenyl)-2,2,6-trimethyl-7-(2-methylhydrazineyl)-7-oxoheptyl)(methyl)carbamate (Intermediate 67-10) with benzyl (R)-(7-(3-iodophenyl)-2,2,7-trimethyl-8-(2-methylhydrazineyl)-8-oxooctyl)(methyl)carbamate (Intermediate 67-11, 3.00 g, 5.18 mmol) and ethyl (E)-3-(6-fluoro-5-((2-(imino(methylthio)methyl)pyridin-4-yl)oxy)-1H-indol-4-yl)acrylate (Intermediate 75) with ethyl 2-(6-fluoro-5-((2-(imino(methylthio)methyl)pyridin-4-yl)oxy)-1-tosyl-1H-indol-4-yl)acetate (Intermediate 75-1, 2.40 g, 4.43 mmol), the reaction procedure sequence (Steps A, B, C, D and F) described for Example 118 was used to prepare the title compound (22 mg, 0.033 mmol) as a white solid. LC-MS: MS (ESI): 681 m/z [M+H]⁺, retention time: 2.35 minutes, purity: >99% (214 nm) (LC-MS Method 3). ¹H NMR (400 MHz, CD₃OD) δ 8.52 (d, J=5.8 Hz, 1H), 8.04 (d, J=2.4 Hz, 1H), 7.31-7.26 (m, 2H), 7.16 (t, J=7.6 Hz, 1H), 7.11 (s, 1H), 7.06 (d, J=7.6 Hz, 1H), 7.00 (d, J=7.6 Hz, 1H), 6.79 (d, J=5.8 Hz, 1H), 6.53 (d, J=3.2 Hz, 1H), 4.31 (s, 3H), 3.82 (s, 2H), 3.51-3.41 (m, 1H), 3.09-2.99 (m, 4H), 2.98-2.89 (m, 1H), 2.69-2.56 (m, 2H), 2.52-2.39 (m, 1H), 1.97-1.85 (m, 2H), 1.72-1.60 (m, 4H), 1.49-1.42 (m, 2H), 1.40-1.24 (m, 2H), 1.09 (d, J=6.4 Hz, 3H), 0.92 (s, 3H), 0.78 (s, 3H).

### Example 122. Compound 122. (2R)-3-[3-[(6R)-23-fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]-2-methyl-propanoic acid

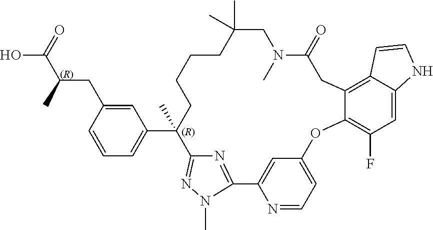

Exchanging benzyl (R)-(6-(3-iodophenyl)-2,2,6-trimethyl-7-(2-methylhydrazineyl)-7-oxoheptyl)(methyl)carbamate (Intermediate 67-10) with benzyl (R)-(7-(3-iodophenyl)-2,2,7-trimethyl-8-(2-methylhydrazineyl)-8-oxooctyl)(methyl)carbamate (Intermediate 67-11, 3.00 g, 5.18 mmol) and ethyl (E)-3-(6-fluoro-5-((2-(imino(methylthio)methyl)pyridin-4-yl)oxy)-1H-indol-4-yl)acrylate (Intermediate 75) with ethyl 2-(6-fluoro-5-((2-(imino(methylthio)methyl)pyridin-4-yl)oxy)-1-tosyl-1H-indol-4-yl)acetate (Intermediate 75-1, 2.40 g, 4.43 mmol) in corresponding Step A, and methyl (2S)-3-iodo-2-methyl-propanoate with methyl (2R)-3-iodo-2-methyl-propanoate (156 mg, 0.686 mmol) in corresponding Step D, the reaction procedure sequence (Steps A, B, C, D and F) described for Example 118 was used to prepare the title compound (84 mg, 0.122 mmol) as a white solid. LC-MS: MS (ESI): 681 m/z [M+H]⁺, retention time: 2.65 minutes, purity: 99% (214 nm) (LC-MS Method 3). ¹H NMR (400 MHz, CD₃OD) δ 8.52 (d, J=5.8 Hz, 1H), 8.03 (d, J=2.4 Hz, 1H), 7.32-7.26 (m, 2H), 7.16 (t, J=7.6 Hz, 1H), 7.11 (s, 1H), 7.06 (d, J=8.0 Hz, 1H), 7.00 (d, J=8.0 Hz, 1H), 6.82-6.80 (m, 1H), 6.53 (d, J=3.2 Hz, 1H), 4.30 (s, 3H), 3.80 (s, 2H), 3.50-3.40 (m, 1H), 3.29-3.22 (m, 1H), 3.08-3.04 (m, 4H), 2.98-2.88 (m, 1H), 2.68-2.57 (m, 2H), 2.50-2.39 (m, 1H), 1.96-1.86 (m, 1H), 1.73-1.59 (m, 4H), 1.53-1.43 (m, 2H), 1.39-1.27 (m, 2H), 1.09 (d, J=6.5 Hz, 3H), 0.92 (s, 3H), 0.79 (s, 3H).

### Example 123. Compound 123. 3-[3-[(6R)-23-Fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]propanoic acid

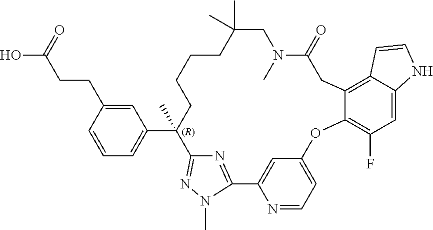

### (6R)-23-Fluoro-6-(3-iodophenyl)-3,6,11,11,13-pentamethyl-20-(p-tolylsulfonyl)-25-oxa-3,4,13,20,29,31-hexazapentacyclo[24.3.1.12,5.016,24.017,21]hentriaconta-1(30),2(31),4,16,18,21,23,26,28-nonaen-14-one

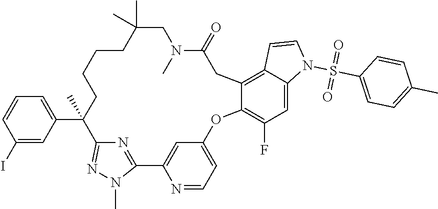 [SMILES: Cc1ccc(S(=O)(=O)n2ccc3c4c(c(F)cc32)Oc2ccnc(c2)-c2nc(nn2C)[C@@](C)(c2cccc(I)c2)CCCCC(C)(C)CN(C)C(=O)C4)cc1]

Step A: Exchanging benzyl (R)-(6-(3-iodophenyl)-2,2,6-trimethyl-7-(2-methylhydrazineyl)-7-oxoheptyl)(methyl)carbamate (Intermediate 67-10) with benzyl (R)-(7-(3-iodophenyl)-2,2,7-trimethyl-8-(2-methylhydrazineyl)-8-oxooctyl)(methyl)carbamate (Intermediate 67-11, 3.00 g, 5.18 mmol) and ethyl (E)-3-(6-fluoro-5-((2-(imino(methylthio)methyl)pyridin-4-yl)oxy)-1H-indol-4-yl)acrylate (Intermediate 75) with ethyl 2-(6-fluoro-5-((2-(imino(methylthio)methyl)pyridin-4-yl)oxy)-1-tosyl-1H-indol-4-yl)acetate (Intermediate 75-1, 2.40 g, 4.43 mmol) in corresponding Step A, the reaction procedure sequence (Steps A, B, C) described for Example 118 was used to prepare the title compound (1.40 g, 1.57 mmol) as a white solid. LC-MS: MS (ESI): 875 m/z [M+H]⁺, retention time: 1.72 minutes, purity: 95% (214 nm) (LC-MS Method 3).

### Ethyl (E)-3-[3-[(6R)-23-fluoro-3,6,11,11,13-pentamethyl-14-oxo-20-(p-tolylsulfonyl)-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]prop-2-enoate

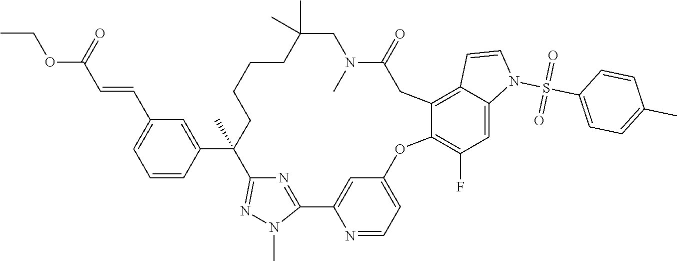 [SMILES: CCOC(=O)/C=C/c1cccc([C@@]2(C)CCCCC(C)(C)CN(C)C(=O)Cc3c(c(F)cc4c3ccn4S(=O)(=O)c3ccc(C)cc3)Oc3ccnc(c3)-c3nc2nn3C)c1]

Step B: To a stirred solution of Step A product (200 mg, 0.229 mmol) in 1,4-dioxane (10 mL) and water (2 mL) were added potassium carbonate (95 mg, 0.686 mmol), ethyl (E)-3-(4,4,5,5-tetramethyl-1,3,2-dioxaborolan-2-yl)acrylate (78 mg, 0.343 mmol) and [1,1′-bis(diphenylphosphino)ferrocene]dichloropalladium(II) (17 mg, 0.0229 mmol). The reaction mixture was heated at 90° C. for 3 hours, quenched with water (15 mL), and extracted with ethyl acetate (2×10 mL). The combined organic phase was washed with brine, dried over sodium sulfate, filtered, and concentrated. The residue was purified by silica gel column chromatography (25 g silica gel column, eluting with 0-50% ethyl acetate in petroleum ether) to give the title compound (180 mg, 0.202 mmol, 88%) as a yellowish oil. LC-MS: MS (ESI): 847 m/z [M+H]⁺, retention time: 2.75 minutes, purity: 95% (214 nm) (LC-MS Method 3).

### (E)-3-[3-[(6R)-23-Fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]prop-2-enoic acid

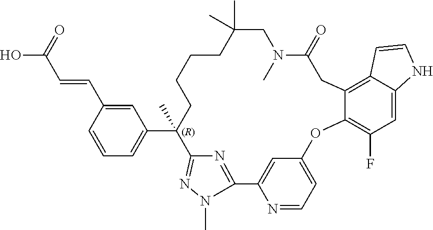

Step C: To a stirred solution of Step B product (180 mg, 0.213 mmol) in tetrahydrofuran (9 mL), methanol (3 mL) and water (2 mL) was added lithium hydroxide monohydrate (91 mg, 2.18 mmol). The reaction mixture was stirred for 16 hours at room temperature, then quenched with water (15 mL), acidified with 1N hydrochloric acid to pH<5, and extracted with ethyl acetate (2×10 mL). The combined organic phase was washed with brine, dried over sodium sulfate, filtered, and concentrated. The residue was purified by automated silica gel column chromatography (25 g silica gel column, eluting with 0-40% ethyl acetate in petroleum ether) to give the title compound (120 mg, 0.175 mmol, 82%) as an oil. LC-MS: MS (ESI): 665 m/z [M+H]⁺, retention time: 1.58 minutes, purity: 97% (254 nm) (LC-MS Method 3).

### 3-[3-[(6R)-23-Fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]propanoic acid

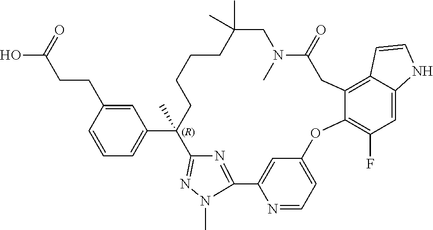

Step D: To a stirred solution of Step C product (116 mg, 0.175 mmol) in ethanol (10 mL) was added palladium on carbon (100 mg, 10%, 50% wet). The mixture was stirred under hydrogen balloon at room temperature for 16 hours and filtered through a pad of Celite. The filtrate was concentrated. The residue was purified using PREP-HPLC to give the title compound (77 mg, 0.116 mmol, 66%) as a white solid. LC-MS: MS (ESI): 667 m/z [M+H]⁺, retention time: 2.54 minutes, purity: >99% (254 nm) (LC-MS Method 3). ¹H NMR (400 MHz, CD₃OD) δ 8.52 (d, J=5.6 Hz, 1H), 8.04 (d, J=2.8 Hz, 1H), 7.31-7.22 (m, 2H), 7.17-7.12 (m, 2H), 7.08-6.99 (m, 2H), 6.83-6.75 (m, 1H), 6.54 (d, J=3.2 Hz, 1H), 4.31 (s, 3H), 3.81 (s, 2H), 3.47-3.38 (m, 1H), 3.10-3.03 (m, 4H), 2.86 (t, J=7.6 Hz, 2H), 2.54 (t, J=7.6 Hz, 2H), 2.51-2.42 (m, 1H), 1.97-1.85 (m, 1H), 1.72-1.60 (m, 4H), 1.52-1.42 (m, 2H), 1.41-1.25 (m, 2H), 1.15-1.05 (m, 1H), 0.92 (s, 3H), 0.78 (s, 3H).

### Example 124. Compound 124. 3-[3-[(6R)-23-fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]-2,2-dimethyl-propanoic acid

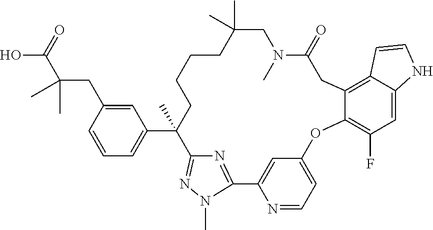 [SMILES: CN1CC(C)(C)CCCC[C@](C)(c2cccc(CC(C)(C)C(=O)O)c2)c2nc(n(C)n2)-c2cc(ccn2)Oc2c(F)cc3[nH]ccc3c2CC1=O]

Exchanging (6R,14E)-23-fluoro-6-(3-iodophenyl)-3,6,10,10,12-pentamethyl-25-oxa-3,4,12,20,29,31-hexazapentacyclo[24.3.1.12,5.016,24.017,21]hentriaconta-1(30),2(31),4,14,16,18,21,23,26,28-decaen-13-one with (6R)-23-fluoro-6-(3-iodophenyl)-3,6,11,11,13-pentamethyl-20-(p-tolylsulfonyl)-25-oxa-3,4,13,20,29,31-hexazapentacyclo [24.3.1.12,5.016,24.017,21]hentriaconta-1(30),2(31),4,16,18,21,23,26,28-nonaen-14-one (Step C product of Example 121, 100 mg, 0.114 mmol), and methyl (2R)-3-iodo-2-methyl-propanoate with methyl 3-iodo-2,2-dimethyl-propanoate (30 mg, 0.126 mmol), the reaction procedure sequence (Steps D and F) described for Example 118 was followed to prepare the title compound (25 mg, 0.0360 mmol) as a white solid. LC-MS: MS (ESI): 695 m/z [M+H]⁺, retention time: 2.11 minutes, purity: >99% (214 nm) (LC-MS Method 25). ¹H NMR (400 MHz, CD₃OD) δ 8.52 (d, J=6.0 Hz, 1H), 8.02 (d, J=2.0 Hz, 1H), 7.29-7.24 (m, 2H), 7.15-7.12 (m, 1H), 7.07-7.05 (m, 2H), 6.97-6.95 (dm, 1H), 6.82-6.80 (m, 1H), 6.53 (d, J=3.2 Hz, 1H), 4.29 (s, 3H), 3.83 (s, 2H), 3.47-3.36 (m, 1H), 3.27-3.22 (m, 1H), 3.06 (s, 3H), 2.79 (s, 2H), 2.47-2.41 (m, 1H), 1.93-1.85 (m, 1H), 1.70-1.60 (m, 4H), 1.47-1.45 (m, 2H), 1.34-1.28 (m, 2H), 1.25-1.19 (m, 1H), 1.09 (s, 6H), 0.91 (s, 3H), 0.78 (s, 3H) ppm.

### Example 125. Compound 125. 1-[[3-[(6R)-23-fluoro-3,6,11,11,13-pentamethyl-14-oxo-25-oxa-3,4,13,20,29,31-hexazapentacyclo[24.3.1.12,5.016,24.017,21]hentriaconta-1(30),2(31),4,16,18,21,23,26,28-nonaen-6-yl]phenyl]methyl]cyclopropanecarboxylic acid

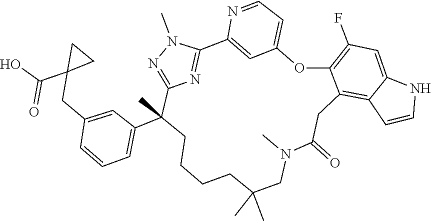 [SMILES: CN1CC(C)(C)CCCC[C@](C)(c2cccc(CC3(C(=O)O)CC3)c2)c2nc(n(C)n2)-c2cc(ccn2)Oc2c(F)cc3[nH]ccc3c2CC1=O]

Exchanging methyl (2R)-3-iodo-2-methyl-propanoate with methyl 1-(iodomethyl)cyclopropanecarboxylate (6.6 mg, 0.0274 mmol), and tris(dibenzylideneacetone)dipalladium/2-dicyclohexylphosphino-2′,6′-dimethoxybiphenyl with bis(tri-t-butylphosphine)palladium(0) (1.2 mg, 0.00229 mmol) in corresponding Step D, the reaction procedure sequence (Steps D and F) described for Example 118 was used to prepare the title compound (22 mg, 0.0320 mmol) as a white solid. LC-MS: MS (ESI): 693 m/z [M+H]⁺, retention time: 1.48 minutes, purity: >99% (214 nm) (LC-MS Method 29). ¹H NMR (400 MHz, CD₃OD): d 8.51 (d, J=5.6 Hz. 1H), 8.04 (d, J=2.4 Hz, 1H), 7.29 (d, J=3.6 Hz, 1H), 7.26 (d, J=10.4 Hz, 1H), 7.18-7.13 (m, 2H), 7.08-7.03 (m, 2H), 6.79-6.77 (m, 1H), 6.54-6.53 (m, 1H), 4.31 (s, 3H), 3.81 (s, 2H), 3.51-3.23 (m, 2H), 3.06 (s, 3H), 2.91 (s, 2H), 2.45-2.40 (m, 1H), 1.95-1.88 (m, 1H), 1.76-1.60 (m, 4H), 1.52-1.47 (m, 2H), 1.40-1.27 (m, 2H), 1.20-1.10 (m, 3H), 0.96 (s, 3H), 0.82-0.73 (m, 5H) ppm.

### Example 126. Compound 126. 3-[3-(23,29-Difluoro-6,10,10-trimethyl-13-oxo-8,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

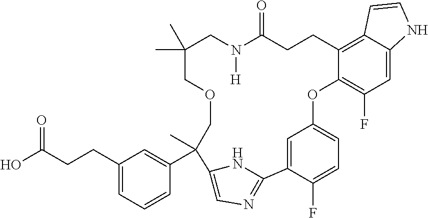 [SMILES: CC1(C)CNC(=O)CCc2c(c(F)cc3[nH]ccc23)Oc2ccc(F)c(c2)-c2ncc([nH]2)C(C)(c2cccc(CCC(=O)O)c2)COC1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 0.96 g, 2.50 mmol), and 8-bromo-6-(3-iodophenyl)-7-oxooctanenitrile (Intermediate 1-1) with benzyl (3-(4-bromo-2-(3-iodophenyl)-2-methyl-3-oxobutoxy)-2,2-dimethylpropyl)carbamate (Intermediate 11-1, 1.5 g, 2.50 mmol), the reaction procedure sequence (Steps A, B, C, F, E and G, in this order) described for Example 1 was used to prepare the title compound (5 mg) as a white solid. MS (ESI): 657 m/z [M+H]⁺. ¹H NMR (400 MHz, CD₃OD) δ 7.40 (m, 1H), 7.33 (d, J=3.2 Hz, 1H), 7.19-7.14 (m, 2H), 7.10-7.07 (m, 3H), 6.97 (m, 3H), 6.56-6.55 (m, 1H), 4.12 (d, J=8.8 Hz, 1H), 3.74 (d, J=8.8 Hz, 1H), 3.17-3.05 (m, 3H), 2.88-2.83 (m, 3H), 2.70-2.66 (m, 2H), 2.53-2.46 (m, 2H), 2.40-2.38 (m, 2H), 1.61 (s, 3H), 0.77 (s, 3H), 0.64 (s, 3H) ppm

### Example 127. Compound 127. 3-[3-(23,29-Difluoro-6,10,12-trimethyl-13-oxo-8,25-dioxa-3,12,20,31-tetrazapentacyclo[24.3.1.12,5.016,24.017,21]hentriaconta-1(30),2,4,16,18,21,23,26,28-nonaen-6-yl)phenyl]propanoic acid

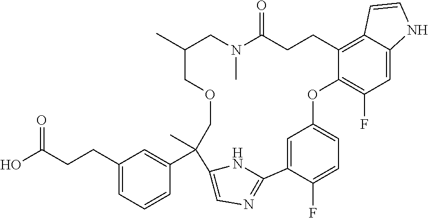 [SMILES: CC1COCC(C)(c2cccc(CCC(=O)O)c2)c2cnc([nH]2)-c2cc(ccc2F)Oc2c(F)cc3[nH]ccc3c2CCC(=O)N(C)C1]

Exchanging methyl (E)-3-(5-(3-carbamimidoyl-4-fluorophenoxy)-6-fluoro-1H-indol-4-yl)acrylate (Intermediate 5) with ethyl (E)-3-(5-(3-carbamimidoyl-4-fluorophenoxy)-6-fluoro-1H-indol-4-yl)acrylate (Intermediate 5-2, 1.9 g, 4.82 mmol), 8-bromo-6-(3-iodophenyl)-7-oxooctanenitrile (Intermediate 1-1) with benzyl (3-(4-bromo-2-(3-iodophenyl)-2-methyl-3-oxobutoxy)-2-methylpropyl)(methyl)carbamate (Intermediate 11-5, 2.9 g, 4.82 mmol) in Step A, the reaction procedure sequence (Steps A, B, C, F, E and G, in this order) described for Example 1 was used to prepare the title compound (123 mg) as a white solid. MS (ESI): 657.3 m/z [M+H]⁺ (a mixture of 4 diastereomers, originated from two chiral centers).

### Biological Assays

### Example 128. Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cystic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada). Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 uL of protein solution was aliquoted into the 384-well plate harboring the test compounds and 10 uL mineral oil was overlaid onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (T_{agg}) was calculated based on the inflection point of the curve. To better compare data across experiments the average T_{agg} for DMSO control wells was calculated and subtracted from values for wells containing compounds to obtain a “ΔT_{agg}” value. These ΔT_{agg} values reflect stabilizing efficacy of the compounds. As set forth in Table 2 below, an ΔT_{agg} value less than or equal to 5° C. is marked “A”; a value above 5° C. and less than or equal to 10° C. is marked “B”; a value greater than 10° C. and less than or equal to 25° C. is marked “C”.

### Example 129. TECC24 AUC Fold Over Benchmark VX-809 @10 μM

The effects of a test agent on CFTR-mediated transepithelial chloride transport is measured using TECC24 recording analysis. Test agents are solubilized in DMSO. Solubilized test agents are mixed with incubation medium containing DMEM/F12, Ultroser G (0.5%; Crescent Chemical, catalog #67042), Hyclone Fetal Clone II (2%; GE Healthcare, catalog #SH30066.02), bovine brain extract (0.25%; Lonza, catalog #CC-4098), insulin (2.5 pg/mL), IL-13 (10 ng/mL), hydrocortisone (20 nM), transferrin (2.5 g/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), are exposed to test agents or controls dissolved in incubation medium. The CF-HBE cells are cultured at 36.5° C. for 48 hours before TECC24 recordings are performed in the presence or absence of test agent, a positive control or vehicle (DMSO).

Following incubation, the transwell cell culture inserts containing the test agent or control-treated CF-HBE cells are loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contain (in mM) 140 NaCl, 5 KCl, 2 CaCl₂), 1 MgCl₂, 10 Hepes, and 10 glucose (adjusted to pH 7.4 with NaOH). To inhibit basal Na+ absorption, the ENaC inhibitor benzamil (10 μM) is added to the bath. Then, the adenylate cyclase activator, forskolin (10 μM), is added to the bath to activate CFTR. The forskolin-stimulated Cl− transport is halted by addition of CFTR inhibitor-172 (20 μM) to the bath at the end of the experiment to confirm specificity. VT and TEER recordings are digitally acquired at routine intervals using TECC or MTECC software (EP Design). VT and TEER are transformed into equivalent transepithelial Cl− current (IEQ), and the Area Under the Curve (AUC) of the IEQ time course between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increases the AUC of the forskolin-stimulated IEQ relative to VX-809 in CF-HBE cells, and this increase is inhibited by CFTR inhibitor-172, then the test agent is considered a CFTR corrector. TECC assay efficacy is expressed as the ratio of the test agent AUC divided by VX-809 AUC. As set forth in Table 2 below, a TECC value less than 3 is marked “A”; a value between 3 and 6 is marked “B”; a value between 6 and 20 is marked “C”.

| Compound No. | DSLS | TECC |
|---|---|---|
| 1 | B | |
| 2 | C | |
| 3 | C | B |
| 4 | C | B |
| 5 | B | |
| 6 | C | |
| 7 | C | |
| 8 | C | A |
| 9 | C | |
| 10 | C | |
| 11 | C | |
| 12 | C | |
| 13 | C | |
| 14 | C | |
| 15 | C | |
| 16 | C | |
| 17 | C | |
| 18 | A | |
| 19 | C | |
| 20A | C | |
| 20B | A | A |
| 20C | C | C |
| 21 | C | |
| 22 | C | |
| 23 | C | |
| 24 | C | |
| 25 | B | |
| 26 | C | B |
| 27 | A | A |
| 28 | C | A |
| 29 | C | B |
| 30 | B | A |
| 31 | A | A |
| 32 | B | |
| 33 | B | A |
| 34 | C | A |
| 35 | A | A |
| 36 | C | B |
| 37 | A | A |
| 38 | A | A |
| 39 | A | A |
| 40A | A | A |
| 40B | C | B |
| 41 | B | B |
| 42A | A | A |
| 42A | C | C |
| 43 | A | A |
| 44 | C | C |
| 45 | C | B |
| 46A | A | A |
| 46B | C | A |
| 47A | A | A |
| 47B | C | A |
| 48A | A | |
| 48B | A | |
| 49A | A | A |
| 49B | C | A |
| 50 | | |
| 51 | | |
| 52 | C | A |
| 53A | A | A |
| 53B | B | A |
| 54 | A | A |
| 55 | B | A |
| 56 | A | A |
| 57 | B | B |
| 58 | A | A |
| 59 | B | A |
| 60A | A | A |
| 60B | B | B |
| 61 | C | B |
| 62 | A | A |
| 63 | B | A |
| 64A | A | A |
| 64B | A | B |
| 65A | A | A |
| 65B | C | A |
| 66A | A | A |
| 66B | C | A |
| 67A | C | C |
| 67B | B | A |
| 68 | B | A |
| 69A | A | B |
| 69B | A | B |
| 70 | C | A |
| 71 | A | A |
| 72A | A | A |
| 72B | B | A |
| 73 | C | B |
| 74 | A | A |
| 75A | B | B |
| 75B | A | A |
| 76A | A | A |
| 76B | C | C |
| 77A | C | C |
| 77B | A | A |
| 78 | A | |
| 79A | A | A |
| 79B | C | C |
| 80 | C | B |
| 81 | C | |
| 82 | A | |
| 83 | A | A |
| 84 | C | B |
| 85 | C | B |
| 86 | C | C |
| 87 | C | B |
| 88 | C | B |
| 89 | C | B |
| 90 | C | B |
| 91 | B | A |
| 92 | C | C |
| 93A | A | B |
| 93B | C | A |
| 94 | B | A |
| 95A | B | B |
| 95B | B | B |
| 96A | C | B |
| 96B | C | C |
| 97A | C | B |
| 97B | C | B |
| 98A | C | C |
| 98B | A | B |
| 99A | C | B |
| 99B | A | B |
| 100A | C | C |
| 100B | A | C |
| 101A | C | B |
| 101B | A | A |
| 102 | B | B |
| 103 | C | A |
| 104 | C | |
| 105 | C | |
| 106 | C | B |
| 107 | C | B |
| 108 | C | A |
| 109 | C | B |
| 110 | C | B |
| 111 | C | C |
| 112 | C | B |
| 113 | C | A |
| 114 | C | A |
| 115 | C | A |
| 116 | C | B |
| 117A | B | A |
| 117B | C | B |
| 118 | C | B |
| 119 | B | B |
| 120 | B | A |
| 121 | C | A |
| 122 | B | A |
| 123 | C | A |
| 124 | A | A |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

We claim:

1. A compound of Formula I: 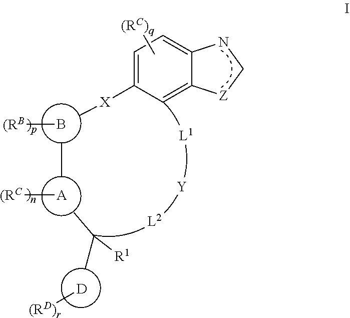
or a pharmaceutically acceptable salt thereof,
wherein
L¹ is an optionally substituted C₁₋₆ alkylene chain wherein 1-3 of the methylene units is optionally and independently replaced by —O—, —N(R²)—, —C(O)—, —S—, —S(O)—, an optionally substituted 3-6 membered carbocyclyl, 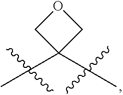 [SMILES: *C1(*)COC1]
or optionally substituted C₂ alkenylene;
L² is an optionally substituted C₁₋₆ alkylene chain wherein 1-3 of the methylene units is optionally and independently replaced by —C(CD₃)₂-, —O—, —N(R²), —C(O)—, —S—, —S(O)—, an optionally substituted 3-6 membered carbocyclyl, or optionally substituted C₂ alkenylene;
Ring A is an optionally substituted 5-membered heteroaryl comprising 1-4 heteroatoms selected from the group consisting of N, O or S;
Ring B is optionally substituted phenyl or optionally substituted 6-membered heteroaryl;
Ring D is optionally substituted phenyl or optionally substituted 5-6-membered heteroaryl;
X is selected from the group consisting of —O—, —S—, —CH₂—, —C(OH)H—, —SO—, —CO—, —SO₂—, —CFH—, —CF₂—, and —N(R²)—;
Y is selected from the group consisting of —S(O)₂N(R²)—, —OC(O)N(R²)—, and —C(O)N(R²)—;
each R^{A} is independently selected from the group consisting of halogen, cyano, optionally substituted C₁-C₆ aliphatic, optionally substituted C₁-C₆ alkoxy, and —CD₃;
each R^{B} is independently selected from the group consisting of halogen, cyano, —C(O)N(R²)₂, C(O)OR², —OR², —N(R²)₂, optionally substituted C₁-C₆ aliphatic and optionally substituted C₁-C₆ alkoxy;
each R^{C} is independently selected from the group consisting of hydrogen, halogen, cyano, optionally substituted C₁-C₆ aliphatic or optionally substituted C₁-C₆ alkoxy;
each R^{D} is independently selected from the group consisting of halogen, cyano, —C(O)N(R²)₂, —C(O)OR², —OR², —N(R²)₂, optionally substituted C₁-C₆ aliphatic, optionally substituted C₁-C₃ alkoxy, optionally substituted 5-6-membered heteroaryl, and optionally substituted 3-6-membered heterocyclyl comprising 1-3 heteroatoms selected from the group consisting of N, O or S, wherein each R^{D} is optionally substituted with 1-6 instances of R^{d};
wherein two instances of R^{D} may be taken together to form an optionally substituted 5-7 membered carbocyclic ring, optionally substituted 5-6-membered heteroaryl, and optionally substituted 3-6-membered heterocyclyl comprising 1-3 heteroatoms selected from the group consisting of N, O or S;
each R^{d} is independently selected from the group consisting of hydrogen, —OH, —CD₃, —C(O)N(R²)₂, C(O)OR², —OR², —N(R²)₂, —S(O)₂R² optionally substituted C₁-C₆ aliphatic, optionally substituted 5-6-membered heteroaryl, and optionally substituted 3-6-membered heterocyclyl comprising 1-3 heteroatoms selected from the group consisting of N, O or S;
R¹ is selected from the group consisting of hydrogen, cyano, —OR², —(CH₂)₀₋₃N(R²)₂, optionally substituted C₁-C₃ aliphatic, 3-6-membered heterocyclyl comprising 1-3 heteroatoms selected from the group consisting of N, O or S, and —CD₃;
each R² is independently selected from hydrogen, optionally substituted C₁-C₆ aliphatic, —OH, C₁-C₆ alkoxy, —S(O)₂(optionally substituted C₁-C₆ aliphatic);
Z is —CH═, —N═ or —NH—;
n is 0, 1, 2 or 3;
p is 0, 1, 2, 3 or 4;
q is 1 or 2; and
r is 0, 1, 2, 3, 4 or 5.

2. The compound of claim 1, wherein Ring D is optionally substituted phenyl or optionally substituted pyridine.

3. The compound of claim 2, wherein Ring D is

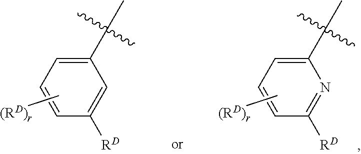

4. The compound of any of the previous claims, wherein Ring D is

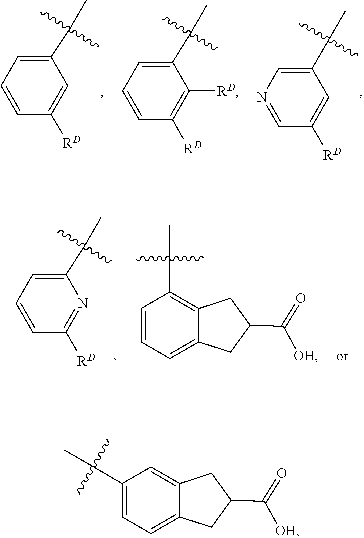

5. The compound of any of the previous claims, wherein Ring D is

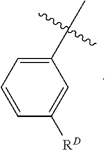

6. The compound of any of the previous claims, wherein Ring B is optionally substituted phenyl, optionally substituted pyridine, or optionally substituted pyridone.

7. The compound of any of the previous claims, wherein Ring B is 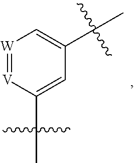
wherein
W is —CH═, —C(R^{B})═ or —N═; and
V is —CH═, —C(R^{B})═ or —N═.

8. The compound of any of the previous claims, wherein Ring B is

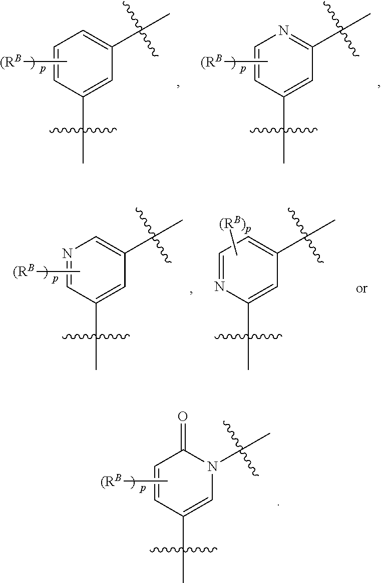

9. The compound of any of the previous claims, wherein Ring B is

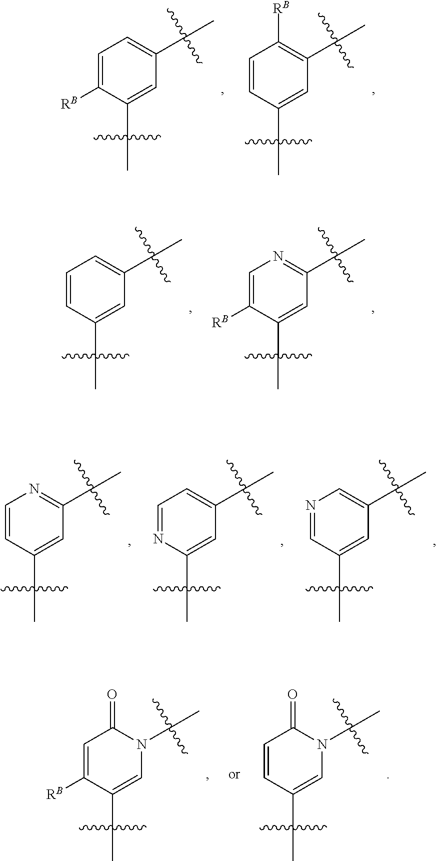

10. The compound of any of the previous claims, wherein Ring B is

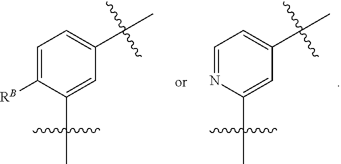

11. The compound of any of the previous claims, wherein R^{B} is halogen or optionally substituted C₁-C₃ alkyl.

12. The compound of any of the previous claims, wherein R^{B} is halogen.

13. The compound of any of the previous claims, wherein R^{B} is fluoro.

14. The compound of any of the previous claims, wherein the compound is of formula (I-a), (I-b), (I-c) or (I-d): 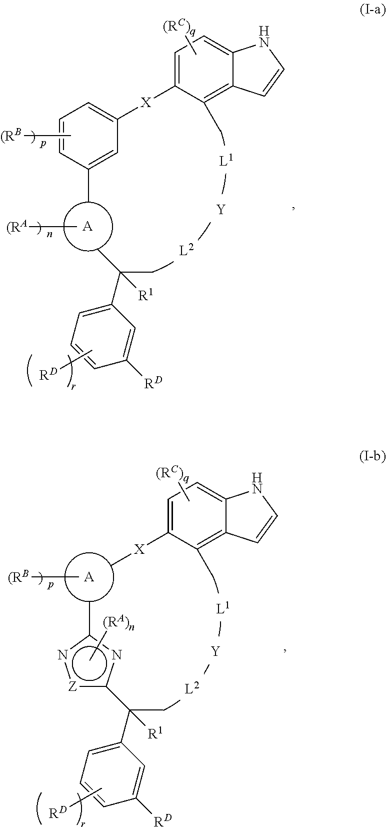 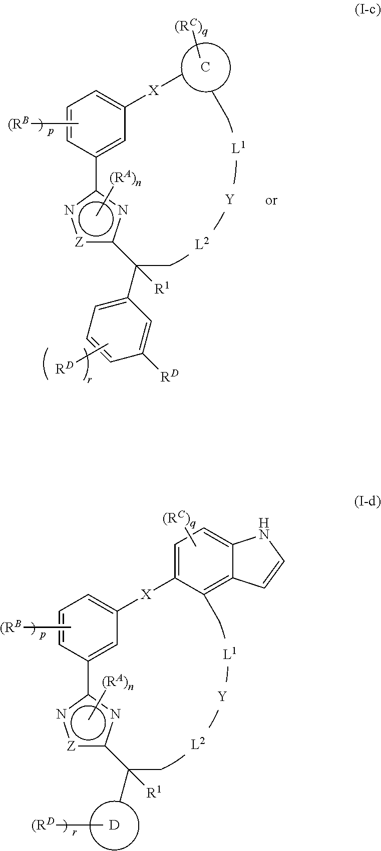
or a pharmaceutically acceptable salt thereof
wherein
Z is C or N.

15. The compound of any of the previous claims wherein the compound is of formula (I-a1), (I-a2), (I-a3), (I-a4), or (I-a5): 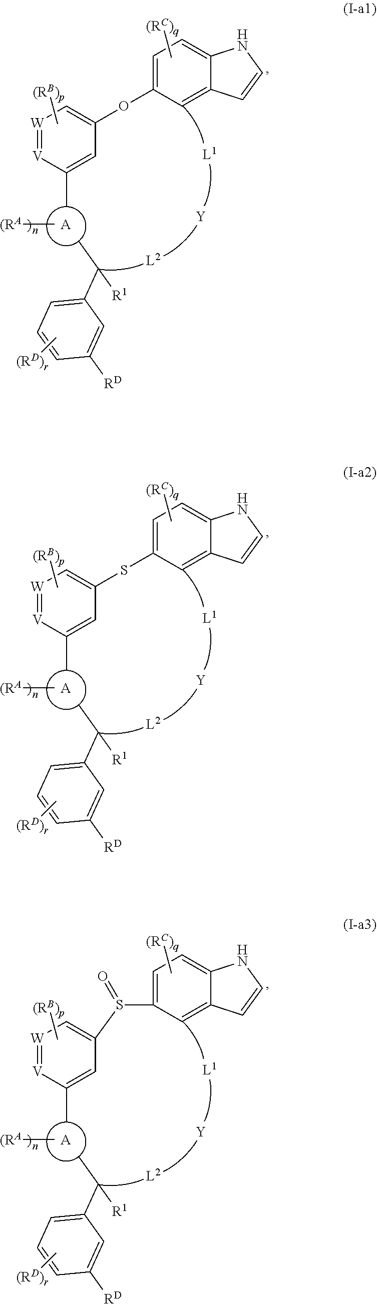 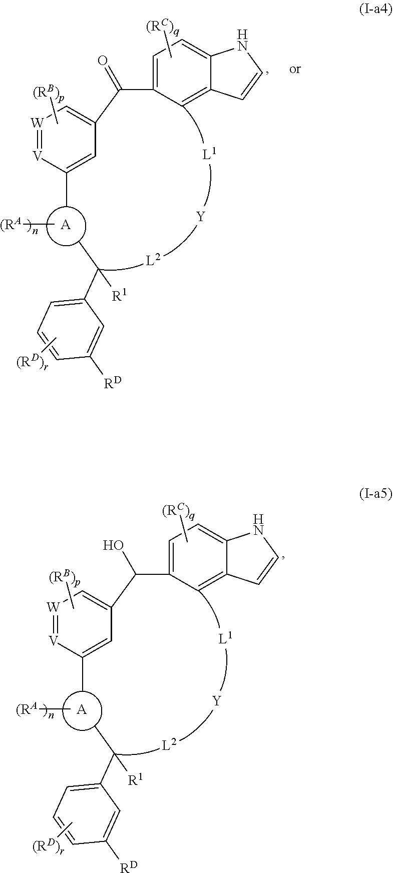
or a pharmaceutically acceptable salt thereof.

16. The compound of any of the previous claims wherein the compound is of formula (I-d1), (I-d2), (I-d3), (I-d4), or (I-d5) 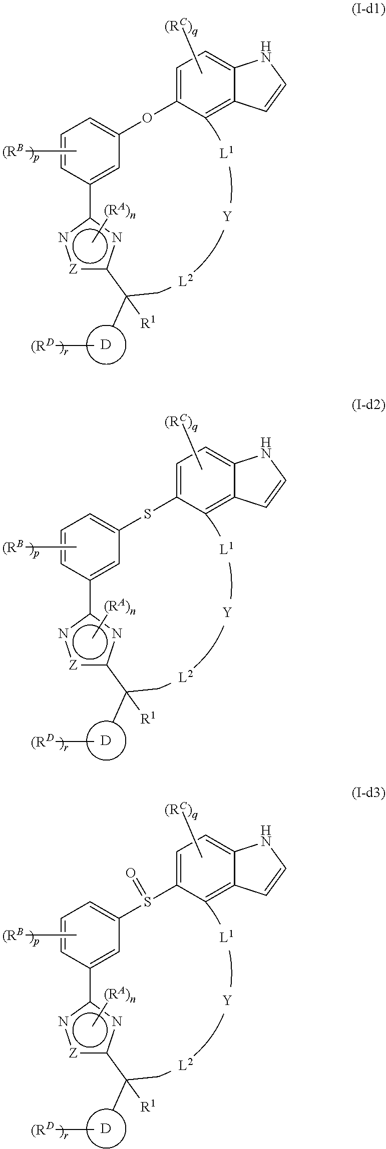 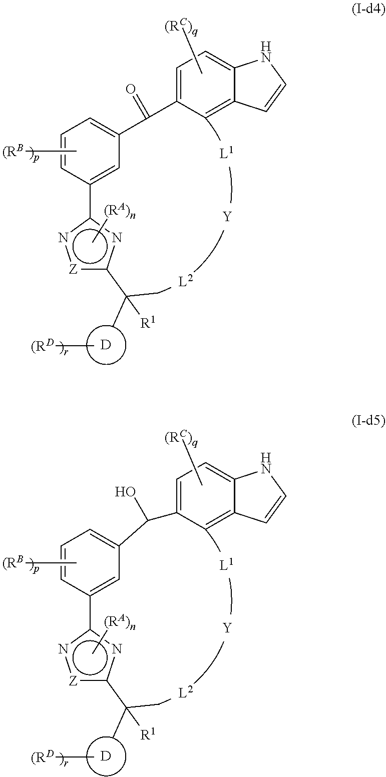
or a pharmaceutically acceptable salt thereof.

17. The compound of any of the previous claims, wherein Ring C is selected from optionally substituted indole, optionally substituted indazole, optionally substituted benzimidazole, optionally substituted 6-azaindole, and optionally substituted 7-azaindole.

18. The compound of any of the previously claims, wherein Ring C is optionally substituted indole.

19. The compound of any the previous claims, wherein at least one instance of R^{D} is 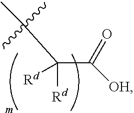
wherein
each R^{d} is independently hydrogen, optionally substituted methyl, —OH, —OMe, or —CD₃;
wherein, two instances R^{d} may, with the atoms on which they are attached, form a cyclopropyl ring; and
m is 0, 1, 2, or 3.

20. The compound of claim 16, wherein each R^{d} is independently hydrogen, methyl, —CF₃, —CF₂H, or —CFH₂.

21. The compound of claim 17, wherein each R^{d} is independently selected from hydrogen and methyl.

22. The compound of any of the previous claims, wherein at least one instance of R^{D} is selected from the group consisting of

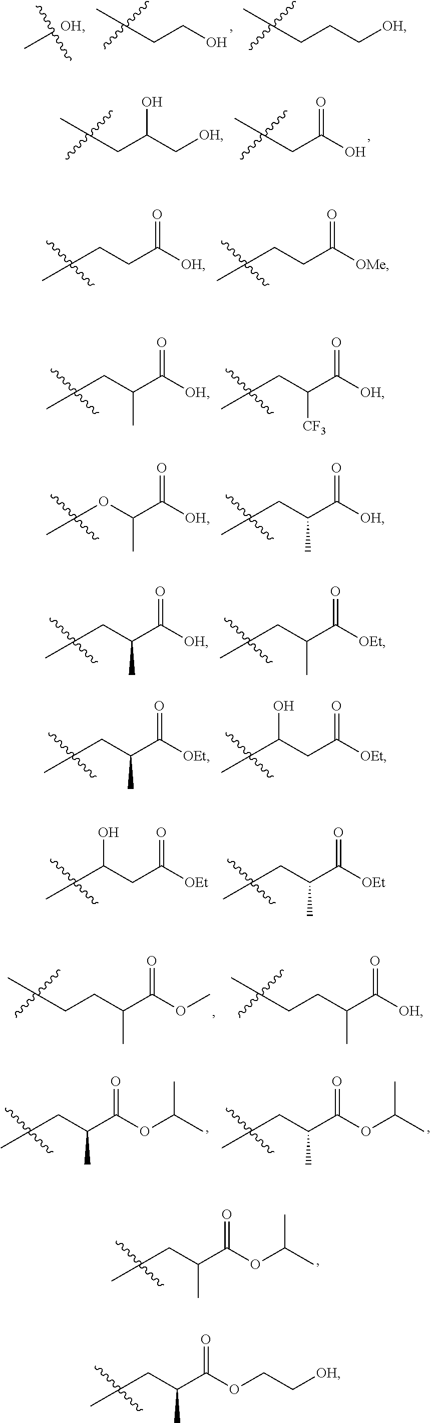

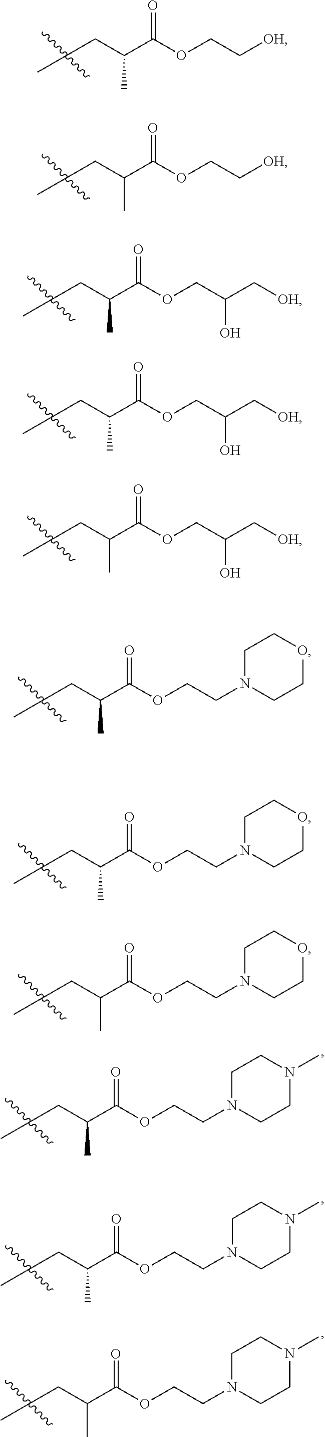

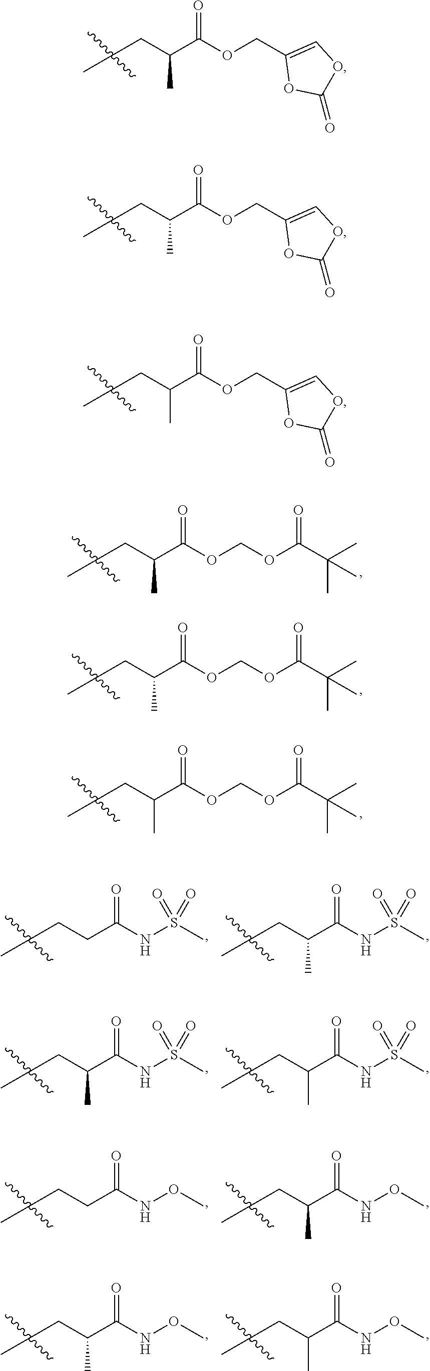

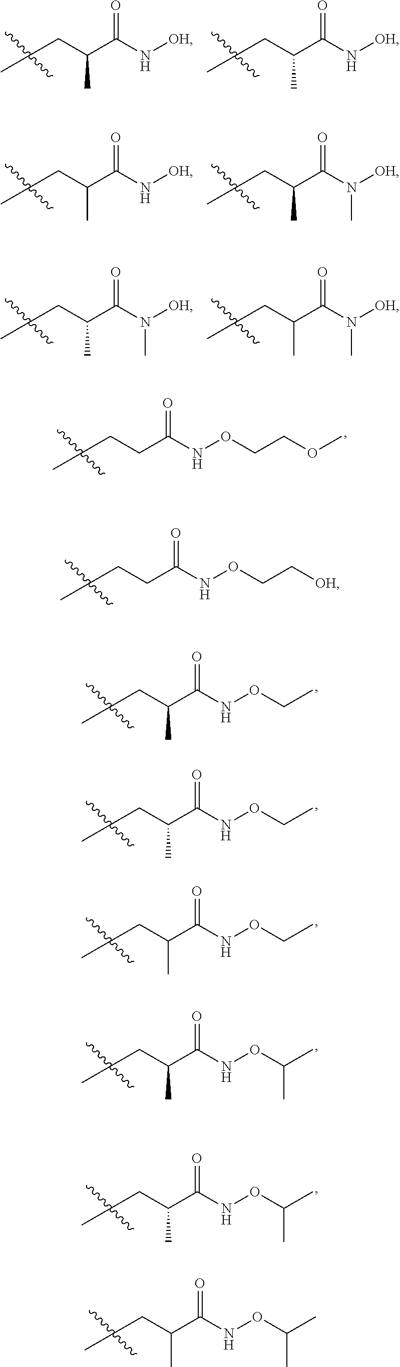

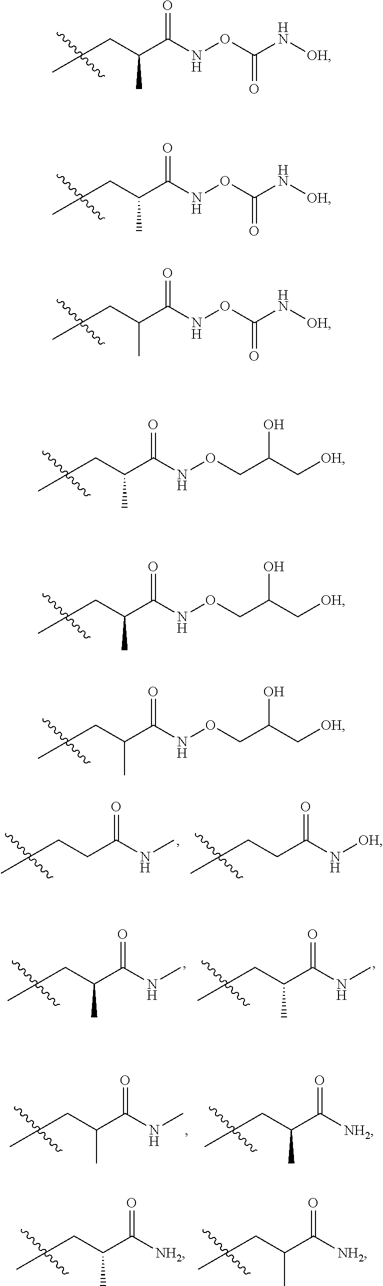

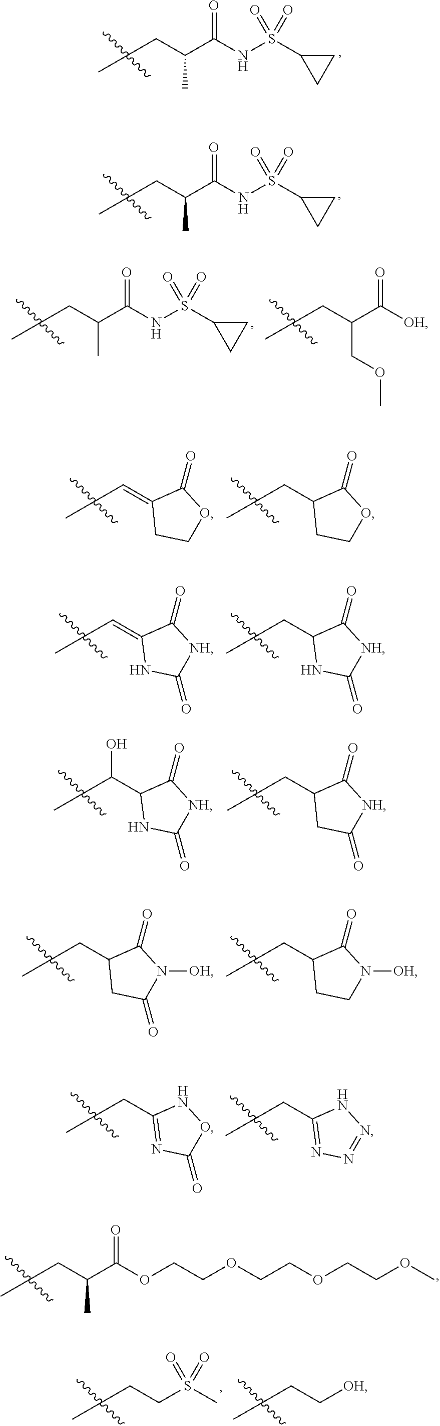

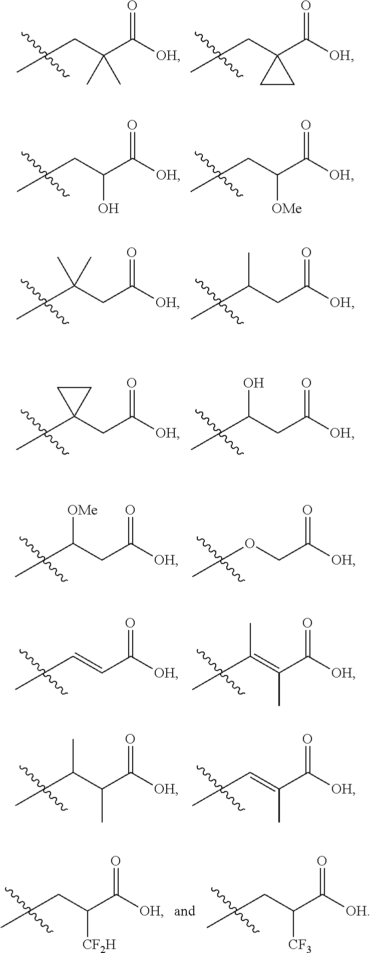

23. The compound of claim 19, wherein at least one instance of R^{D} is selected from the group consisting of

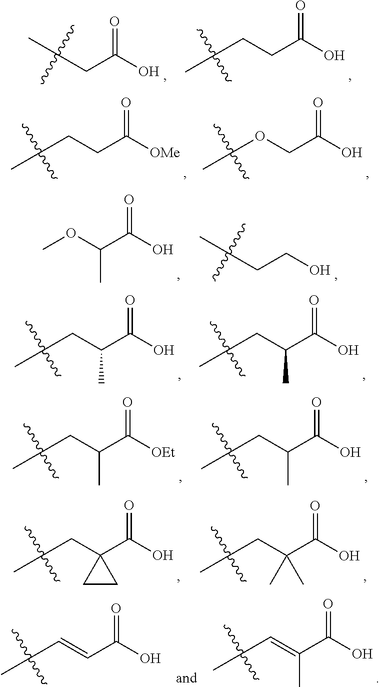

24. The compound of claim 20, wherein R^{D} is

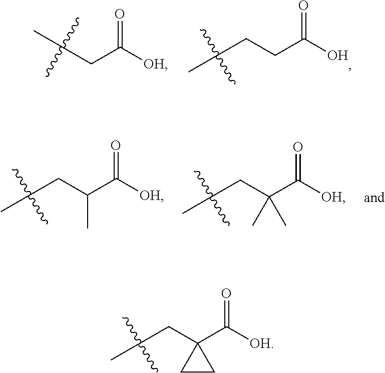

25. The compound of claim 21, wherein R^{D} is

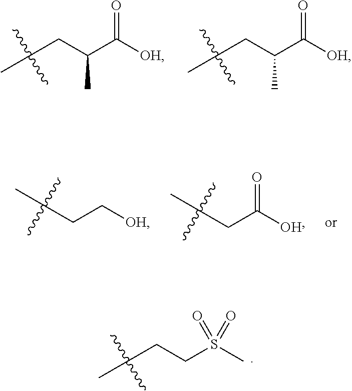

26. The compound of claim 21, wherein R^{D} is

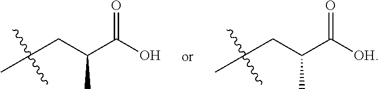

27. The compound of any of the previous claims wherein, ring A is selected from the group consisting of imidazole, pyrazole, tetrazole, oxazole, thiazole, and 1, 2, 4 triazole.

28. The compound of any of the previous claims, wherein each R^{A} is independently selected from hydrogen, methyl and —CD₃.

29. The compound of any of the previous claims, wherein the compound is of formula (I-e): 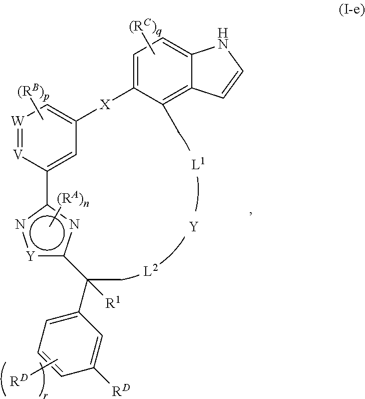
or a pharmaceutically acceptable salt thereof.

30. The compound of any of the previous claims, wherein L¹ is an optionally substituted C₁₋₆ alkylene chain and L² is an optionally substituted C₁₋₆ alkylene chain, wherein one of the methylene units of L² is optionally replaced with —O—.

31. The compound of any of the previous claims, wherein L¹ is a C₁₋₆ alkylene chain substituted with 1-3 instances of methyl, and L² is C₁₋₆ alkylene chain, wherein one of the methylene units of L² is optionally replaced with —O— and wherein L² is optionally substituted with 1-3 instances of methyl.

32. The compound of any of the previous claims, wherein L¹ is an unsubstituted C₂ alkylene chain.

33. The compound of any of the previous claims, wherein L² is an C₅ alkylene chain, wherein one of the methylene units of L² is optionally replaced with —O— and wherein L² is optionally substituted with 1-3 instances of methyl.

34. The compound of any of the previous claims, wherein L² is a C₅ alkylene chain, wherein L² is optionally substituted with 1-3 instances of methyl.

35. The compound of any of the previous claims, wherein L² is a C₅ alkylene chain, wherein L² is optionally substituted with 1-3 instances of methyl.

36. The compound of any of the previous claims, wherein the compound is of formula (I-f): 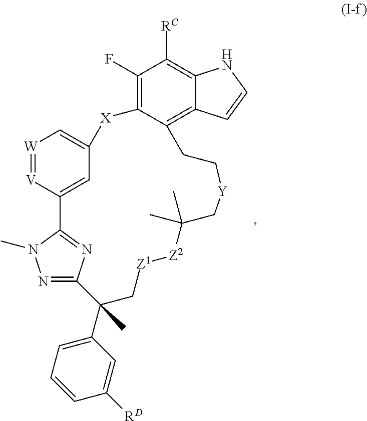
or a pharmaceutically acceptable salt thereof
wherein Z¹ is —CH₂— or —O—; and
Z² is is —CH₂— or —O—.

37. The compound of any of the previous claims, wherein the compound is of formula (I-g) 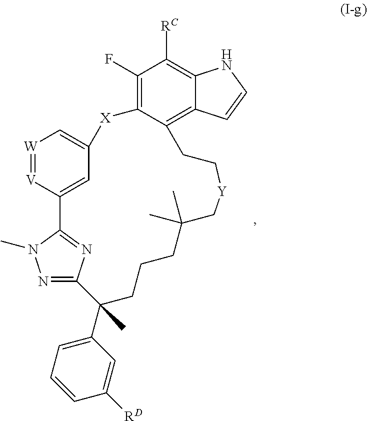
or a pharmaceutically acceptable salt thereof.

38. The compound of any of the previous claims, wherein the compound is of formula (1-g1) or (I-g2) 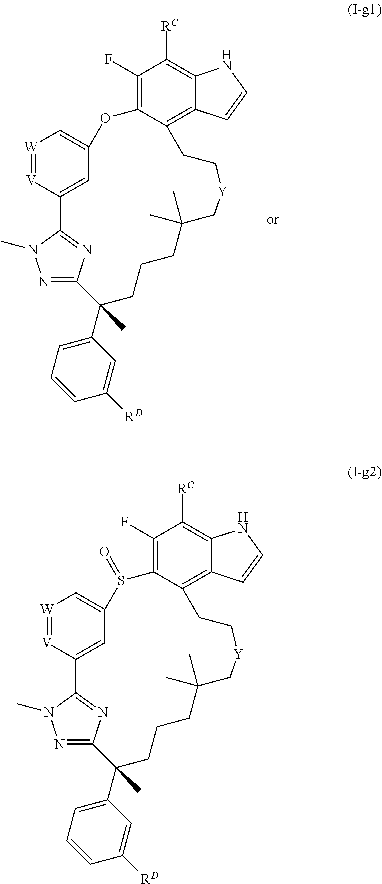
or a pharmaceutically acceptable salt thereof.

39. The compound of any of the previous claims, wherein the compound is of formula (I-h): 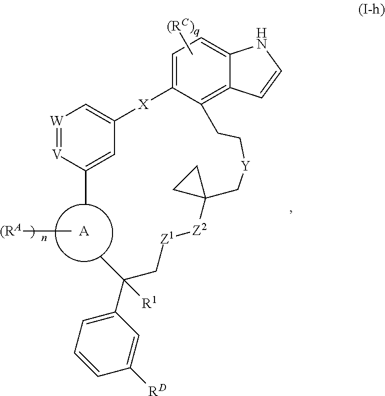
or a pharmaceutically acceptable salt thereof.

40. The compound of any of the previous claims, wherein the compound is of formula (I-i) 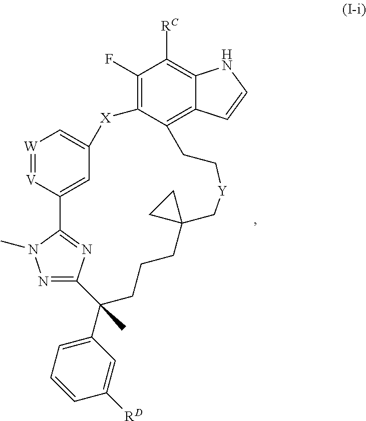
or a pharmaceutically acceptable salt thereof.

41. The compound of any of the previous claims, wherein Z¹ is —O— and Z² is —CH₂—.

42. The compound of any of the previous claims, wherein Z¹ is —CH₂— and Z² is —O—.

43. The compound of any of the previous claims, wherein Z¹ is —CH₂— and Z² is —CH₂—.

44. The compound of any of the previous claims, wherein R¹ is optionally substituted C₁-C₃ aliphatic.

45. The compound of any of the previous claims, wherein R¹ is optionally substituted methyl or —CD₃.

46. The compound of any of the previous claims, wherein each R^{d} is independently hydrogen or optionally substituted C₁-C₃ aliphatic.

47. The compound of any of the previous claims, wherein at least one R^{d} is optionally substituted C₁-C₃ aliphatic.

48. The compound of any of the previous claims, wherein at least one R^{d} is methyl.

49. The compound of any of the previous claims, wherein each R^{A} is independently hydrogen or optionally substituted C₁-C₃ aliphatic.

50. The compound of any of the previous claims, wherein each R^{A} is independently hydrogen, methyl or —CD₃.

51. The compound of any of the previous claims, wherein R^{C} is hydrogen, halogen.

52. The compound of any of the previous claims, wherein R^{C} is halogen.

53. The compound of any of the previous claims, wherein R^{C} is hydrogen.

54. The compound of any of the previous claims, wherein R^{D} is halogen.

55. The compound of any of the previous claims, wherein R^{D} is fluoro.

56. The compound of any of the previous claims, wherein R^{C} is fluoro.

57. The compound of any of the previous claims, wherein Y is selected from the group consisting of —S(O)₂N(H)—, —OC(O)N(H)—, and —C(O)N(H)—.

58. The compound of any of the previous claims, wherein Y is selected from the group consisting of —S(O)₂N(Me)-, —OC(O)N(Me)-, and —C(O)N(Me)-.

59. A compound selected from a compound of Table 1 or a pharmaceutically acceptable salt thereof.

60. A pharmaceutical composition comprising a compound of any of the previous claims and a pharmaceutically acceptable excipient.

61. A method of treating a CFTR-mediated disease or disorder comprising administering a patient in need there of a compound any of claims 1-61 or a pharmaceutical composition of claim 58.

62. The method of claim 61, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

64. The method of claim 61 or 62, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

65. The method of any one of claims 61-63, wherein the disease or condition is cystic fibrosis.

66. A method of treating cystic fibrosis in a subject, comprising administering to the subject a therapeutically effective amount of a compound of any one of claims 1-59, or the pharmaceutical composition of claim 60.

67. The method of claim 65, wherein the subject is human.
